(19) United States
(12) Patent Application Publication
(10) Pub. No.: US 2026/0015598 A1
(43) Pub. Date: Jan. 15, 2026

(54) COMPOSITIONS AND METHODS FOR NUCLEIC ACID MODIFICATIONS

(71) Applicant: ACRIGEN BIOSCIENCES, Berkeley, CA (US)

(72) Inventors: David Rabuka, Berkeley, CA (US); Michael Schelle, Berkeley, CA (US); Allison Sharrar, Berkeley, CA (US)

(21) Appl. No.: 18/873,215

(22) PCT Filed: Jun. 9, 2023
(86) PCT No.: PCT/US2023/068191

§ 371 Date: Dec. 9, 2024

Related U.S. Application Data
(60) Provisional application No. 63/351,140, filed on Jun. 10, 2022.
(60) Provisional application No. 63/383,107, filed on Nov. 10, 2022.
(60) Provisional application No. 63/482,936, filed on Feb. 2, 2023.

(51) Int. Cl. C12N 9/22; C12N 15/11; C12N 15/86; C12N 15/90

(52) U.S. Cl. CPC C12N 9/226; C12N 15/11; C12N 15/86; C12N 15/907; C12N 2310/20; C12N 2750/14143

(57) ABSTRACT

The present disclosure provides nucleases and compositions, methods, and systems thereof for nucleic acid modification. More particularly, the present disclosure provides compositions and system comprising a nuclease comprising an amino acid sequence having at least 70% identity to any of SEQ ID NOs: 1-250 and at least one gRNA for target nucleic acid modification.

### CROSS-REFERENCE TO RELATED APPLICATIONS

This application claims the benefit of U.S. Provisional Application Nos. 63/351,140, filed Jun. 10, 2022, 63/383,107, filed Nov. 10, 2022, and 63/482,936, filed Feb. 2, 2023, the contents of which are herein incorporated by reference in their entirety.

### FIELD

The present invention relates to nucleases and compositions, methods, and systems thereof for nucleic acid modification.

### SEQUENCE LISTING STATEMENT

The contents of the electronic sequence listing titled ACRIG_404894_601.xml (Size: 579,833 bytes; and Date of Creation: Jun. 8, 2023) is herein incorporated by reference in its entirety.

### BACKGROUND

Clustered regularly interspaced short palindromic repeats (CRISPR)-associated (Cas) nucleases dominate the nucleic acid-editing landscape because they are versatile, rapid, and easy-to-use editing tools. The most well-characterized CRISPR-Cas nuclease, Cas9, utilizes one or more RNAs to act as a sequence-specific targeting element linking the nuclease to the target nucleic acid. However, presently CRISPR/Cas systems have some limitations for use, particularly in eukaryotic organisms including low efficiency of editing, off-target events, target sequence preferences and efficient delivery and expression of the nuclease.

### SUMMARY

Provided herein are compositions comprising a nuclease, wherein the nuclease comprises a sequence with at least 70%, 80%, 85%, 90%, 95%, 96%, 97%, 98%, 99% or greater than 99% identity to any one of SEQ ID NOs: 1-250. In some embodiments, the amino acid sequence of the nuclease comprises any one of SEQ ID NOs: 1-250.

In some embodiments, the nuclease further comprises a nuclear localization sequence (NLS). In some embodiments, the NLS is at the N-terminus, C-terminus or both the N-terminus and C-terminus of the nuclease. In some embodiments, the NLS at the N-terminus and the NLS at the C-terminus of the nuclease are different sequences.

Also provided are nucleic acid molecules comprising a first polynucleotide sequence encoding the nuclease and vectors comprising the nucleic acid molecules. In some embodiments, the vector further comprises a promoter operatively linked to the first polynucleotide sequence. In some embodiments, the vector further comprises a second polynucleotide sequence encoding a guide RNA (gRNA). In some embodiments, the vector further comprises a promoter operatively linked to the second polynucleotide.

In some embodiments, the gRNA comprises a sequence with at least 60%, 70%, 80%, 85%, 90%, 95%, 96%, 97%, 98%, 99% identity or 100% identity to any one of SEQ ID NOs: 251-422. In some embodiments, the gRNA comprises any one of SEQ ID NOs: 251-343. In some embodiments, the gRNA comprises any one of SEQ ID NOs: 344-422. In some embodiments, the gRNA comprises any one of SEQ ID NOs: 472-482. In some embodiments, the gRNA comprises SEQ ID NO: 346, 420, 481, or 479.

In some embodiments, the gRNA comprises a tracr sequence and the gRNA comprises one or more sequence deletions in or near the region encompassing the tracr sequence. In some embodiments, the one or more sequence deletions comprises sequences predicted to form a stem-loop structure. In some embodiments, the one or more sequence deletions comprises sequences predicted to form a stem-loop structure at or near the 5′ end of the gRNA. In some embodiments, the gRNA comprises SEQ ID NO: 346, 420, 481, or 479.

In some embodiments, the gRNA comprises a spacer sequence of at least 18 nucleotides in length. In some embodiments, the gRNA comprises a spacer sequence between 18 and 20 nucleotides in length.

In some embodiments, the nuclease comprises SEQ ID NO: 20, and the at least one gRNA comprises a sequence with at least 60%, 70%, 80%, 85%, 90%, 95%, 96%, 97%, 98%, 99% identity or 100% identity to any one of SEQ ID NOs: 309, 346, 352, 358, 362-364, 380, 392-395, 410-420, 472-479, and 481. In some embodiments, the nuclease comprises SEQ ID NO: 20, and the at least one gRNA comprises a sequence with at least 60%, 70%, 80%, 85%, 90%, 95%, 96%, 97%, 98%, 99% identity or 100% identity to any one of 352, 358, 363, 364, 380, 392, and 417. In some embodiments, the nuclease comprises SEQ ID NO: 20, and the at least one gRNA comprises a sequence with at least 60%, 70%, 80%, 85%, 90%, 95%, 96%, 97%, 98%, 99% identity or 100% identity to any one of SEQ ID NOs: 346 and 362. In some embodiments, the nuclease comprises SEQ ID NO: 20, and the at least one gRNA comprises a sequence with at least 60%, 70%, 80%, 85%, 90%, 95%, 96%, 97%, 98%, 99% identity or 100% identity to any one of SEQ ID NOs: 410-419.

In some embodiments, the nuclease comprises a sequence having at least 70%, 80%, 85%, 90%, 95%, 96%, 97%, 98%, or 99% identity to SEQ ID NO: 20, and wherein the at least one gRNA comprises any one of SEQ ID NOs: 309, 346, 352, 358, 362-364, 380, 392-395, 410-420, 472-479 and 481. In some embodiments, the nuclease comprises SEQ ID NO: 20, and the at least one gRNA comprises a sequence with at least 60%, 70%, 80%, 85%, 90%, 95%, 96%, 97%, 98%, 99% identity or 100% identity to any one of SEQ ID NOs: 352, 358, 363, 364, 380, 392, and 417. In some embodiments, the nuclease comprises SEQ ID NO: 20, and the at least one gRNA comprises a sequence with at least 60%, 70%, 80%, 85%, 90%, 95%, 96%, 97%, 98%, 99% identity or 100% identity to any one of SEQ ID NOs: 346 and 362. In some embodiments, the nuclease comprises SEQ ID NO: 20, and the at least one gRNA comprises a sequence with at least 60%, 70%, 80%, 85%, 90%, 95%, 96%, 97%, 98%, 99% identity or 100% identity to any one of SEQ ID NOs: 410-419

In some embodiments, the nuclease comprises SEQ ID NO: 21, and the at least one gRNA comprises a sequence with at least 60%, 70%, 80%, 85%, 90%, 95%, 96%, 97%, 98%, 99% identity or 100% identity to any one of SEQ ID NOs: 310, 344-349, 361-366, 404-422 and 479-482. In some embodiments, the nuclease comprises a sequence having at least 70%, 80%, 85%, 90%, 95%, 96%, 97%, 98%, or 99% identity to SEQ ID NO: 21, and wherein the at least one gRNA comprises any one of SEQ ID NOs: 310, 344-349, 361-366, 404-422, and 479-482.

In some embodiments, the nuclease comprises SEQ ID NO: 22, and the at least one gRNA comprises a sequence with at least 60%, 70%, 80%, 85%, 90%, 95%, 96%, 97%, 98%, 99% identity or 100% identity to any one of SEQ ID NOs: 311, 346, 381, and 398-399. In some embodiments, the nuclease comprises a sequence having at least 70%, 80%, 85%, 90%, 95%, 96%, 97%, 98%, or 99% identity to SEQ ID NO: 22, and wherein the at least one gRNA comprises any one of SEQ ID NOs: 311, 346, 381, and 398-399.

In some embodiments, the nuclease comprises SEQ ID NO: 23, and the at least one gRNA comprises a sequence with at least 60%, 70%, 80%, 85%, 90%, 95%, 96%, 97%, 98%, 99% identity or 100% identity to any one of SEQ ID NOs: 312, 346, and 382. In some embodiments, the nuclease comprises a sequence having at least 70%, 80%, 85%, 90%, 95%, 96%, 97%, 98%, or 99% identity to SEQ ID NO: 23, and wherein the at least one gRNA comprises any one of SEQ ID NOs: 312, 346, and 382.

In some embodiments, the nuclease comprises SEQ ID NO: 24, and the at least one gRNA comprises a sequence with at least 60%, 70%, 80%, 85%, 90%, 95%, 96%, 97%, 98%, 99% identity or 100% identity to any one of SEQ ID NOs: 310, 313, 325, 346, 350-355, 358, 361-363, 367-372, and 389-392. In some embodiments, the nuclease comprises SEQ ID NO: 24, and the at least one gRNA comprises a sequence with at least 60%, 70%, 80%, 85%, 90%, 95%, 96%, 97%, 98%, 99% identity or 100% identity to any one of SEQ ID NOs: 346, 352, 358, 361, 362, 368, 369, and 392.

In some embodiments, the nuclease comprises a sequence having at least 70%, 80%, 85%, 90%, 95%, 96%, 97%, 98%, or 99% identity to SEQ ID NO: 24, and wherein the at least one gRNA comprises any one of SEQ ID NOs: 310, 313, 325, 346, 350-355, 358, 361-363, 367-372, and 389-392. In some embodiments, the nuclease comprises a sequence having at least 70%, 80%, 85%, 90%, 95%, 96%, 97%, 98%, or 99% identity to SEQ ID NO: 24, and wherein the at least one gRNA comprises any one of SEQ ID NOs: 346, 352, 358, 361, 362, 368, 369, and 392.

In some embodiments, the nuclease comprises SEQ ID NO: 25, and the at least one gRNA comprises a sequence with at least 60%, 70%, 80%, 85%, 90%, 95%, 96%, 97%, 98%, 99% identity or 100% identity to any one of SEQ ID NOs: 314, 346, 383, and 400. In some embodiments, the nuclease comprises a sequence having at least 70%, 80%, 85%, 90%, 95%, 96%, 97%, 98%, or 99% identity to SEQ ID NO: 25, and wherein the at least one gRNA comprises any one of SEQ ID NOs: 314, 346, 383, and 400.

In some embodiments, the nuclease comprises SEQ ID NO: 26, and the at least one gRNA comprises a sequence with at least 60%, 70%, 80%, 85%, 90%, 95%, 96%, 97%, 98%, 99% identity or 100% identity to any one of SEQ ID NOs: 315, 346, 384, 392, 396-397, 420, 479, and 481. In some embodiments, the nuclease comprises SEQ ID NO: 26, and the at least one gRNA comprises a sequence with at least 60%, 70%, 80%, 85%, 90%, 95%, 96%, 97%, 98%, 99% identity or 100% identity to any one of SEQ ID NOs: 346, 384 and 392.

In some embodiments, the nuclease comprises a sequence having at least 70%, 80%, 85%, 90%, 95%, 96%, 97%, 98%, or 99% identity to SEQ ID NO: 26, and wherein the at least one gRNA comprises any one of SEQ ID NOs: 315, 346, 384, 392, 396-397, 420, 479, and 481. In some embodiments, the nuclease comprises a sequence having at least 70%, 80%, 85%, 90%, 95%, 96%, 97%, 98%, or 99% identity to SEQ ID NO: 26, and wherein the at least one gRNA comprises any one of SEQ ID NOs: 346, 384 and 392.

In some embodiments, the nuclease comprises SEQ ID NO: 27, and the at least one gRNA comprises a sequence with at least 60%, 70%, 80%, 85%, 90%, 95%, 96%, 97%, 98%, 99% identity or 100% identity to any one of SEQ ID NOs: 316, 346, 385, and 401. In some embodiments, the nuclease comprises a sequence having at least 70%, 80%, 85%, 90%, 95%, 96%, 97%, 98%, or 99% identity to SEQ ID NO: 27, and wherein the at least one gRNA comprises any one of SEQ ID NOs: 316, 346, 385, and 401.

In some embodiments, the nuclease comprises SEQ ID NO: 28, and the at least one gRNA comprises a sequence with at least 60%, 70%, 80%, 85%, 90%, 95%, 96%, 97%, 98%, 99% identity or 100% identity to any one of SEQ ID NOs: 317, 346, 386, and 402. In some embodiments, the nuclease comprises a sequence having at least 70%, 80%, 85%, 90%, 95%, 96%, 97%, 98%, or 99% identity to SEQ ID NO: 28, and wherein the at least one gRNA comprises any one of SEQ ID NOs: 317, 346, 386, and 402.

In some embodiments, the nuclease comprises SEQ ID NO: 29, and the at least one gRNA comprises a sequence with at least 60%, 70%, 80%, 85%, 90%, 95%, 96%, 97%, 98%, 99% identity or 100% identity to any one of SEQ ID NOs: 318, 346, 387, and 403. In some embodiments, the nuclease comprises a sequence having at least 70%, 80%, 85%, 90%, 95%, 96%, 97%, 98%, or 99% identity to SEQ ID NO: 29, and wherein the at least one gRNA comprises any one of SEQ ID NOs: 318, 346, 387, and 403.

In some embodiments, the nuclease comprises SEQ ID NO: 36, and the at least one gRNA comprises a sequence with at least 60%, 70%, 80%, 85%, 90%, 95%, 96%, 97%, 98%, 99% identity or 100% identity to any one of SEQ ID NOs: 310, 313, 325, 346, 356-360, and 373-378. In some embodiments, the nuclease comprises a sequence having at least 70%, 80%, 85%, 90%, 95%, 96%, 97%, 98%, or 99% identity to SEQ ID NO: 36, and wherein the at least one gRNA comprises any one of SEQ ID NOs: 310, 313, 325, 346, 356-360, and 373-378.

Additionally provided are systems for modifying a first target nucleic acid comprising: a) a nuclease comprising an amino acid sequence having 70%, 80%, 85%, 90%, 95%, 96%, 97%, 98%, 99%, greater than 99% or 100% identity to any of SEQ ID NOs: 1-250 or a first nucleic acid sequence encoding the nuclease; and b) at least one guide RNA (gRNA) comprising a sequence complementary to at least a portion of the first target nucleic acid and a region that associates with the nuclease, or a nucleic acid encoding the at least one gRNA.

In some embodiments, the nuclease is capable of recognizing a protospacer adjacent motif (PAM) sequence selected from the group comprising ATTA, GTTA, ATTG, GTTG, TTTA, TTTG, CTTA, and CTTG. In some embodiments, the gRNA comprises a spacer sequence complementary to a first strand sequence of the target nucleic acid, and wherein the first strand sequence is directly adjacent to a protospacer adjacent motif (PAM) sequence selected from the group comprising ATTA, GTTA, ATTG, GTTG, TTTA, TTTG, CTTA, and CTTG. In some embodiments, the PAM sequence comprises DTTR, wherein D is A, G, or T and R is A or G.

In some embodiments, the nuclease is capable of preferentially modifying a first target nucleic acid comprising PAM sequence ATTA as compared to the first target nucleic acid comprising PAM sequence TTTR, wherein R is A or G.

In some embodiments, the nuclease is capable of a higher efficiency of modification of the target nucleic acid as compared to the efficiency of modification by nuclease SEQ ID NO: 471 of the target nucleic acid, wherein the target nucleic acid comprises PAM sequence is ATTA.

In some embodiments, the nuclease in the presence of the gRNA is capable of modifying the first target nucleic acid. In some embodiments, modifying comprises nucleic acid cleavage. In some embodiments, modifying comprises one or more of modification of the target nucleic acid, modulation of transcription from the target nucleic acid, and modification of a polypeptide associated with a target nucleic acid.

In some embodiments, the nuclease further comprises a nuclear localization sequence (NLS). In some embodiments, the NLS is at the N-terminus, C-terminus or both the N-terminus and C-terminus of the nuclease. In some embodiments, the NLS at the N-terminus and the NLS at the C-terminus of the nuclease are different sequences. In some embodiments, the nuclease further comprises a purification tag.

In some embodiments, the gRNA further comprises a sequence complementary to at least a portion of a second target nucleic acid.

In some embodiments, the gRNA comprises a sequence with at least 60%, 70%, 80%, 85%, 90%, 95%, 96%, 97%, 98%, 99% identity or 100% identity to any one of SEQ ID NOs: 251-422. In some embodiments, the gRNA comprises any one of SEQ ID NOs: 251-343. In some embodiments, the gRNA comprises any one of SEQ ID NOs: 344-422. In some embodiments, the gRNA comprises any one of SEQ ID NOs: 472-482. In some embodiments, the gRNA comprises SEQ ID NO: 346, 420, 481, or 479.

In some embodiments, the gRNA comprises a tracr sequence and the gRNA comprises one or more sequence deletions in or near the region encompassing the tracr sequence. In some embodiments, the one or more sequence deletions comprises sequences predicted to form a stem-loop structure. In some embodiments, the one or more sequence deletions comprises sequences predicted to form a stem-loop structure at or near the 5′ end of the gRNA. In some embodiments, the gRNA comprises SEQ ID NO: 346, 420, 481, or 479.

In some embodiments, the gRNA comprises a spacer sequence of at least 18 nucleotides in length. In some embodiments, the gRNA comprises a spacer sequence between 18 and 20 nucleotides in length.

In some embodiments, the nuclease comprises SEQ ID NO: 20, and the at least one gRNA comprises a sequence with at least 60%, 70%, 80%, 85%, 90%, 95%, 96%, 97%, 98%, 99% identity or 100% identity to any one of SEQ ID NOs: 309, 346, 352, 358, 362-364, 380, 392-395, 410-420, 472-479, and 481. In some embodiments, the nuclease comprises SEQ ID NO: 20, and the at least one gRNA comprises a sequence with at least 60%, 70%, 80%, 85%, 90%, 95%, 96%, 97%, 98%, 99% identity or 100% identity to any one of 352, 358, 363, 364, 380, 392, and 417. In some embodiments, the nuclease comprises SEQ ID NO: 20, and the at least one gRNA comprises a sequence with at least 60%, 70%, 80%, 85%, 90%, 95%, 96%, 97%, 98%, 99% identity or 100% identity to any one of SEQ ID NOs: 346 and 362. In some embodiments, the nuclease comprises SEQ ID NO: 20, and the at least one gRNA comprises a sequence with at least 60%, 70%, 80%, 85%, 90%, 95%, 96%, 97%, 98%, 99% identity or 100% identity to any one of SEQ ID NOs: 410-419.

In some embodiments, the nuclease comprises a sequence having at least 70%, 80%, 85%, 90%, 95%, 96%, 97%, 98%, or 99% identity to SEQ ID NO: 20, and wherein the at least one gRNA comprises any one of SEQ ID NOs: 309, 346, 352, 358, 362-364, 380, 392-395, 410-420, 472-479, and 481. In some embodiments, the nuclease comprises SEQ ID NO: 20, and the at least one gRNA comprises a sequence with at least 60%, 70%, 80%, 85%, 90%, 95%, 96%, 97%, 98%, 99% identity or 100% identity to any one of SEQ ID NOs: 352, 358, 363, 364, 380, 392, and 417. In some embodiments, the nuclease comprises SEQ ID NO: 20, and the at least one gRNA comprises a sequence with at least 60%, 70%, 80%, 85%, 90%, 95%, 96%, 97%, 98%, 99% identity or 100% identity to any one of SEQ ID NOs: 346 and 362. In some embodiments, the nuclease comprises SEQ ID NO: 20, and the at least one gRNA comprises a sequence with at least 60%, 70%, 80%, 85%, 90%, 95%, 96%, 97%, 98%, 99% identity or 100% identity to any one of SEQ ID NOs: 410-419

In some embodiments, the nuclease comprises SEQ ID NO: 21, and the at least one gRNA comprises a sequence with at least 60%, 70%, 80%, 85%, 90%, 95%, 96%, 97%, 98%, 99% identity or 100% identity to any one of SEQ ID NOs: 310, 344-349, 361-366, 404-422, and 479-482. In some embodiments, the nuclease comprises a sequence having at least 70%, 80%, 85%, 90%, 95%, 96%, 97%, 98%, or 99% identity to SEQ ID NO: 21, and wherein the at least one gRNA comprises any one of SEQ ID NOs: 310, 344-349, 361-366, 404-422, and 479-482.

In some embodiments, the nuclease comprises SEQ ID NO: 22, and the at least one gRNA comprises a sequence with at least 60%, 70%, 80%, 85%, 90%, 95%, 96%, 97%, 98%, 99% identity or 100% identity to any one of SEQ ID NOs: 311, 346, 381, and 398-399. In some embodiments, the nuclease comprises a sequence having at least 70%, 80%, 85%, 90%, 95%, 96%, 97%, 98%, or 99% identity to SEQ ID NO: 22, and wherein the at least one gRNA comprises any one of SEQ ID NOs: 311, 346, 381, and 398-399.

In some embodiments, the nuclease comprises SEQ ID NO: 23, and the at least one gRNA comprises a sequence with at least 60%, 70%, 80%, 85%, 90%, 95%, 96%, 97%, 98%, 99% identity or 100% identity to any one of SEQ ID NOs: 312, 346, and 382. In some embodiments, the nuclease comprises a sequence having at least 70%, 80%, 85%, 90%, 95%, 96%, 97%, 98%, or 99% identity to SEQ ID NO: 23, and wherein the at least one gRNA comprises any one of SEQ ID NOs: 312, 346, and 382.

In some embodiments, the nuclease comprises SEQ ID NO: 24, and the at least one gRNA comprises a sequence with at least 60%, 70%, 80%, 85%, 90%, 95%, 96%, 97%, 98%, 99% identity or 100% identity to any one of SEQ ID NOs: 310, 313, 325, 346, 350-355, 358, 361-363, 367-372, and 389-392. In some embodiments, the nuclease comprises SEQ ID NO: 24, and the at least one gRNA comprises a sequence with at least 60%, 70%, 80%, 85%, 90%, 95%, 96%, 97%, 98%, 99% identity or 100% identity to any one of SEQ ID NOs: 346, 352, 358, 361, 362, 368, 369, and 392.

In some embodiments, the nuclease comprises a sequence having at least 70%, 80%, 85%, 90%, 95%, 96%, 97%, 98%, or 99% identity to SEQ ID NO: 24, and wherein the at least one gRNA comprises any one of SEQ ID NOs: 310, 313, 325, 346, 350-355, 358, 361-363, 367-372, and 389-392. In some embodiments, the nuclease comprises a sequence having at least 70%, 80%, 85%, 90%, 95%, 96%, 97%, 98%, or 99% identity to SEQ ID NO: 24, and wherein the at least one gRNA comprises any one of SEQ ID NOs: 346, 352, 358, 361, 362, 368, 369, and 392.

In some embodiments, the nuclease comprises SEQ ID NO: 25, and the at least one gRNA comprises a sequence with at least 60%, 70%, 80%, 85%, 90%, 95%, 96%, 97%, 98%, 99% identity or 100% identity to any one of SEQ ID NOs: 314, 346, 383, and 400. In some embodiments, the nuclease comprises a sequence having at least 70%, 80%, 85%, 90%, 95%, 96%, 97%, 98%, or 99% identity to SEQ ID NO: 25, and wherein the at least one gRNA comprises any one of SEQ ID NOs: 314, 346, 383, and 400.

In some embodiments, the nuclease comprises SEQ ID NO: 26, and the at least one gRNA comprises a sequence with at least 60%, 70%, 80%, 85%, 90%, 95%, 96%, 97%, 98%, 99% identity or 100% identity to any one of SEQ ID NOs: 315, 346, 384, 392, 396-397, 420, 479, and 481. In some embodiments, the nuclease comprises SEQ ID NO: 26, and the at least one gRNA comprises a sequence with at least 60%, 70%, 80%, 85%, 90%, 95%, 96%, 97%, 98%, 99% identity or 100% identity to any one of SEQ ID NOs: 346, 384 and 392.

In some embodiments, the nuclease comprises a sequence having at least 70%, 80%, 85%, 90%, 95%, 96%, 97%, 98%, or 99% identity to SEQ ID NO: 26, and wherein the at least one gRNA comprises any one of SEQ ID NOs: 315, 346, 384, 392, 396-397, 420, 479, and 481. In some embodiments, the nuclease comprises a sequence having at least 70%, 80%, 85%, 90%, 95%, 96%, 97%, 98%, or 99% identity to SEQ ID NO: 26, and wherein the at least one gRNA comprises any one of SEQ ID NOs: 346, 384 and 392.

In some embodiments, the nuclease comprises SEQ ID NO: 27, and the at least one gRNA comprises a sequence with at least 60%, 70%, 80%, 85%, 90%, 95%, 96%, 97%, 98%, 99% identity or 100% identity to any one of SEQ ID NOs: 316, 346, 385, and 401. In some embodiments, the nuclease comprises a sequence having at least 70%, 80%, 85%, 90%, 95%, 96%, 97%, 98%, or 99% identity to SEQ ID NO: 27, and wherein the at least one gRNA comprises any one of SEQ ID NOs: 316, 346, 385, and 401.

In some embodiments, the nuclease comprises SEQ ID NO: 28, and the at least one gRNA comprises a sequence with at least 60%, 70%, 80%, 85%, 90%, 95%, 96%, 97%, 98%, 99% identity or 100% identity to any one of SEQ ID NOs: 317, 346, 386, and 402. In some embodiments, the nuclease comprises a sequence having at least 70%, 80%, 85%, 90%, 95%, 96%, 97%, 98%, or 99% identity to SEQ ID NO: 28, and wherein the at least one gRNA comprises any one of SEQ ID NOs: 317, 346, 386, and 402.

In some embodiments, the nuclease comprises SEQ ID NO: 29, and the at least one gRNA comprises a sequence with at least 60%, 70%, 80%, 85%, 90%, 95%, 96%, 97%, 98%, 99% identity or 100% identity to any one of SEQ ID NOs: 318, 346, 387, and 403. In some embodiments, the nuclease comprises a sequence having at least 70%, 80%, 85%, 90%, 95%, 96%, 97%, 98%, or 99% identity to SEQ ID NO: 29, and wherein the at least one gRNA comprises any one of SEQ ID NOs: 318, 346, 387, and 403.

In some embodiments, the nuclease comprises SEQ ID NO:36, and the at least one gRNA comprises a sequence with at least 60%, 70%, 80%, 85%, 90%, 95%, 96%, 97%, 98%, 99% identity or 100% identity to any one of SEQ ID NOs: 310, 313, 325, 346, 356-360, and 373-378. In some embodiments, the nuclease comprises a sequence having at least 70%, 80%, 85%, 90%, 95%, 96%, 97%, 98%, or 99% identity to SEQ ID NO: 36, and wherein the at least one gRNA comprises any one of SEQ ID NOs: 310, 313, 325, 346, 356-360, and 373-378.

In some embodiments, the nucleic acid molecule encoding each one or both of the nuclease and the gRNA is a DNA molecule, such as a vector, plasmid, or linear nucleic acid. In some embodiments the nuclease is encoded in a messenger RNA. In some embodiments, the gRNA is comprised in a small RNA.

In some embodiments, the nuclease and the gRNA are encoded on the same nucleic acid. In some embodiments, the nuclease and the gRNA are encoded on different nucleic acids.

Also provided are vectors comprising the disclosed system. In some embodiments, the vector further comprises a first promoter operatively linked to the nucleic acid encoding the nuclease and a second promoter operatively linked to the nucleic acid encoding the at least one gRNA. In some embodiments, the vector is a viral vector. In some embodiments, the viral vector is an AAV vector. In some embodiments, the first promoter and the second promoter are active in a mammalian cell.

In some embodiments, the system further comprises a target nucleic acid.

In some embodiments, the system is a cell-free system.

Also provided are cells comprising the disclosed compositions and systems. In some embodiments, the cell is a prokaryotic cell. In some embodiments, the cell is a eukaryotic cell (e.g., a mammalian cell or a human cell).

Further provided are methods for modifying a target nucleic acid comprising contacting the target nucleic acid with a nuclease, composition, vector, or system described herein.

In some embodiments, the target nucleic acid sequence is in a cell. In some embodiments, the cell is a prokaryotic cell. In some embodiments, the cell is a eukaryotic cell (e.g., a mammalian cell or a human cell).

In some embodiments, introducing the system or composition into the cell comprises administering the system or composition to a subject. In some embodiments, administering comprises in vivo administration.

Kits comprising any or all of the components of the compositions or systems described herein are also provided. In some embodiments, the kit further comprises one or more reagent, shipping and/or packaging containers, one or more buffers, a delivery device, instructions, software, a computing device, or a combination thereof.

Other aspects and embodiments of the disclosure will be apparent in light of the following detailed description.

### BRIEF DESCRIPTION OF THE DRAWINGS

FIG. 1 is graphs of the editing activity in human cells for nucleases with SEQ ID NOs: 21, 24 and 36, with sgRNAs of SEQ ID NOs: 310, 131, and 325, respectively.

FIG. 2 is a graph of the editing activity in human cells for nucleases with SEQ ID NO: 21 (1-8), SEQ ID NO: 24 (9-16), and SEQ ID NO: 36 (17-24) using single guide RNA (sgRNA) with varying lengths.

FIG. 3 is a graph of the editing activity for Kim-T1 target with a single guide RNA (sgRNA) of SEQ ID NO: 346.

FIG. 4 is a graph of the editing activity with an off-target panel of sgRNA, each of which contains a mismatch at the indicated location.

FIGS. SA-5D are graphs of the editing activity for nucleases of SEQ ID NO: 20 (FIGS. 5A and 5D), SEQ ID NO: 24 (FIG. 5B) and SEQ ID NO: 26 (FIG. 5C) for Kim-T1 target with sgRNAs. FIG. 5E is a schematic of tracrRNA (SEQ ID NO: 508) predicted structure for truncations of middle regions of the third and main RNA stem.

FIG. 6 is a graph of the editing activity for nucleases of SEQ ID NO: 20, 24, and 26, and Un1Cas12f1 across different genomic target sequences.

FIG. 7A is schematics of tracrRNA predicted structures with a full repeat (top; SEQ ID NO: 509) and truncated repeat (bottom; SEQ ID NO: 510) modified from SEQ ID NO: 346. FIG. 7B is a graph of the editing efficiency for SEQ ID NO: 20 with tracrRNAs shown in FIG. 7A for Kim-T1 target. FIG. 7C is a schematic of a tracrRNA (SEQ ID NO: 508) predicted structure with stem stability and A-kink modifications modified from SEQ ID NO: 346. FIGS. 7D and 7E are graphs of the editing efficiencies for nucleases of SEQ ID NO: 24 and 20, respectively, with modified tracrRNAs as indicated for Kim-T1 target.

FIG. 8 is a graph of the editing efficiency of different length spacers (as indicated) for nucleases of SEQ ID NO: 20. Un1Cas12f1 is used as a positive control and NT stands for non-targeted cells, used to determine the level of detection (LOD).

FIGS. 9A and 9B are graphs of editing efficiencies for nucleases of SEQ ID NO: 20 and 26 and the indicated spacer sequences.

FIG. 10 is a schematic of a representative AAV vector design.

FIG. 11 is a graph of editing efficiencies of AAV constructs encoding nuclease of SEQ ID NO: 20 with different guides. Guides shown here are: PCSK9_1=GSp380, PCSK9_2=GSp376, PCSK9_3=GSp377, TTR_1=GSp368, TTR_2=GSp356, PRSS1=GSp342, SMN2=GSp251.

FIG. 12 is a graph of the comparison of editing with AAV and nuclease of SEQ ID NO: 20 with different targets with and without etoposide treatment. NT are samples that had no AAV added to them but were treated, amplified, and sequenced using the same method as AAV treated samples.

### DETAILED DESCRIPTION

The disclosed compositions, systems, kits, and methods comprise nucleases useful for nucleic acid modification. The disclosed nucleases allow for gene editing with improved efficacy and safety for use in in vivo and ex vivo applications of eukaryotic (e.g., mammalian (e.g., human)) therapeutics, diagnostics, and research.

Section headings as used in this section and the entire disclosure herein are merely for organizational purposes and are not intended to be limiting.

### Definitions

The terms “comprise(s),” “include(s),” “having,” “has,” “can,” “contain(s),” and variants thereof, as used herein, are intended to be open-ended transitional phrases, terms, or words that do not preclude the possibility of additional acts or structures. As used herein, comprising a certain sequence or a certain SEQ ID NO usually implies that at least one copy of said sequence is present in recited peptide or polynucleotide. However, two or more copies are also contemplated. The singular forms “a,” “and” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Unless otherwise defined herein, scientific, and technical terms used in connection with the present disclosure shall have the meanings that are commonly understood by those of ordinary skill in the art. For example, any nomenclature used in connection with, and techniques of cell and tissue culture, molecular biology, microbiology, genetics and protein and nucleic acid chemistry and hybridization described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

As used herein, “nucleic acid” or “nucleic acid sequence” refers to a polymer or oligomer of pyrimidine and/or purine bases, preferably cytosine, thymine, and uracil, and adenine and guanine, respectively (See Albert L. Lehninger, Principles of Biochemistry, at 793-800 (Worth Pub. 1982)). The present technology contemplates any deoxyribonucleotide, ribonucleotide, or peptide nucleic acid component, and any chemical variants thereof, such as methylated, hydroxymethylated, or glycosylated forms of these bases, and the like. The polymers or oligomers may be heterogenous or homogenous in composition and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states. In some embodiments, a nucleic acid or nucleic acid sequence comprises other kinds of nucleic acid structures such as, for instance, a DNA/RNA helix, peptide nucleic acid (PNA), morpholino nucleic acid (see, e.g., Braasch and Corey, Biochemistry, 41(14): 4503-4510 (2002)) and U.S. Pat. No. 5,034,506), locked nucleic acid (LNA; see Wahlestedt et al., Proc. Natl. Acad. Sci. U.S.A., 97: 5633-5638 (2000)), cyclohexenyl nucleic acids (see Wang, J. Am. Chem. Soc., 122: 8595-8602 (2000)), and/or a ribozyme. Hence, the term “nucleic acid” or “nucleic acid sequence” may also encompass a chain comprising non-natural nucleotides, modified nucleotides, and/or non-nucleotide building blocks that can exhibit the same function as natural nucleotides (e.g., “nucleotide analogs”); further, the term “nucleic acid sequence” as used herein refers to an oligonucleotide, nucleotide or polynucleotide, and fragments or portions thereof, and to DNA or RNA of genomic or synthetic origin, which may be single or double-stranded, and represent the sense or antisense strand. The terms “nucleic acid,” “polynucleotide,” “nucleotide sequence,” and “oligonucleotide” are used interchangeably. They refer to a polymeric form of nucleotides of any length, either deoxyribonucleotides or ribonucleotides, or analogs thereof.

Nucleic acid or amino acid sequence “identity,” as described herein, can be determined by comparing a nucleic acid or amino acid sequence of interest to a reference nucleic acid or amino acid sequence. The percent identity is the number of nucleotides or amino acid residues that are the same (e.g., that are identical) as between the sequence of interest and the reference sequence divided by the length of the longest sequence (e.g., the length of either the sequence of interest or the reference sequence, whichever is longer). A number of mathematical algorithms for obtaining the optimal alignment and calculating identity between two or more sequences are known and incorporated into a number of available software programs. Examples of such programs include CLUSTAL-W, T-Coffee, and ALIGN (for alignment of nucleic acid and amino acid sequences), BLAST programs (e.g., BLAST 2.1, BL2SEQ, and later versions thereof) and FASTA programs (e.g., FASTA3×, FAS™, and SSEARCH) (for sequence alignment and sequence similarity searches). Sequence alignment algorithms also are disclosed in, for example, Altschul et al., J. Molecular Biol., 215(3): 403-410 (1990), Beigert et al., Proc. Natl. Acad. Sci. USA, 106(10): 3770-3775 (2009), Durbin et al., eds., Biological Sequence Analysis: Probabilistic Models of Proteins and Nucleic Acids, Cambridge University Press, Cambridge, UK (2009), Soding, Bioinformatics, 21(7): 951-960 (2005), Altschul et al., Nucleic Acids Res., 25(17): 3389-3402 (1997), and Gusfield, Algorithms on Strings, Trees and Sequences, Cambridge University Press, Cambridge UK (1997)).

The terms “non-naturally occurring,” “engineered,” and “synthetic” are used interchangeably and indicate the involvement of the hand of man. The terms, when referring to nucleic acid molecules or polypeptides mean that the nucleic acid molecule or the polypeptide is at least substantially free from at least one other component with which it is naturally associated in nature and as found in nature, and/or the nucleic acid molecule or the polypeptide is associated with at least one other component with which it is not naturally associated in nature and/or that there is one or more changes in nucleic acid or amino acid sequence as compared with such sequence as it is found in nature.

A “vector” or “expression vector” is a replicon, such as plasmid, phage, virus, or cosmid, to which another DNA segment, e.g., an “insert,” may be attached or incorporated so as to bring about the replication of the attached segment in a cell.

A cell has been “genetically modified,” “transformed,” or “transfected” by exogenous DNA, e.g., a recombinant expression vector, when such DNA has been introduced inside the cell. The presence of the exogenous DNA results in permanent or transient genetic change. The transforming DNA may or may not be integrated (covalently linked) into the genome of the cell. For example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones that comprise a population of daughter cells containing the transforming DNA. A “clone” is a population of cells derived from a single cell or common ancestor by mitosis. A. “cell line” is a clone of a primary cell that is capable of stable growth in vitro for many generations.

The term “contacting” as used herein refers to bring or put in contact, to be in or come into contact. The term “contact” as used herein refers to a state or condition of touching or of immediate or local proximity. Contacting a composition to a target destination, such as, but not limited to, an organ, tissue, cell, or tumor, may occur by any means of administration known to the skilled artisan.

As used herein, the terms “providing,” “administering,” and “introducing,” are used interchangeably herein and refer to the placement of the composition or systems of the disclosure into a cell, organism, or subject by a method or route which results in at least partial localization to a desired site. The composition or systems can be administered by any appropriate route which results in delivery to a desired location in the cell, organism, or subject.

Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

### Nucleases

Advances and developments in CRISPR-Cas genome editing tools including nucleases and other Cas protein drive major advances in nucleic acid editing. Nucleic acid editing has many uses including in the diagnostics and therapeutics field. Such breadth is accompanied by a diversity of nucleic acid targets and environments in which to engineer editing activity. As such, there is a need for diverse and additional nucleases and associated methods that provide a toolbox for nucleic acid editing.

Disclosed herein are compositions that include nucleases that have Cas-like activity. The disclosed nucleases comprise a sequence having at least 70% identity (e.g., at least 75%, at least 80%, at least 85%, at least 90%, at least 93%, at least 95%, at least 98%, at least 99%, or 100% identity) to an amino acid sequence of SEQ ID NOs: 1-250. In some embodiments, the nuclease comprises a sequence having at least 90% identity an amino acid sequence of SEQ ID NOs: 1-250. In certain embodiments, the nuclease comprises an amino acid sequence of SEQ ID NOs: 1-250.

Any of the nucleases described herein may comprise one or more (e.g., 1, 2, 3, 4, 5, 6, 7, 8, 9, 10, 15, 20, 25, 50, 100, 150, etc.) amino acid substitutions. An amino acid “replacement” or “substitution” refers to the replacement of one amino acid at a given position or residue by another amino acid at the same position or residue within a polypeptide sequence. Amino acids are broadly grouped as “aromatic” or “aliphatic.” An aromatic amino acid includes an aromatic ring. Examples of “aromatic” amino acids include histidine (H or His), phenylalanine (F or Phe), tyrosine (Y or Tyr), and tryptophan (W or Trp). Non-aromatic amino acids are broadly grouped as “aliphatic.” Examples of “aliphatic” amino acids include glycine (G or Gly), alanine (A or Ala), valine (V or Val), leucine (L or Leu), isoleucine (I or Ile), methionine (M or Met), serine (S or Ser), threonine (T or Thr), cysteine (C or Cys), proline (P or Pro), glutamic acid (E or Glu), aspartic acid (A or Asp), asparagine (N or Asn), glutamine (Q or Gin), lysine (K or Lys), and arginine (R or Arg).

The amino acid replacement or substitution can be conservative, semi-conservative, or non-conservative. The phrase “conservative amino acid substitution” or “conservative mutation” refers to the replacement of one amino acid by another amino acid with a common property. A functional way to define common properties between individual amino acids is to analyze the normalized frequencies of amino acid changes between corresponding proteins of homologous organisms (Schulz and Schirmer, Principles of Protein Structure, Springer-Verlag, New York (1979)). According to such analyses, groups of amino acids may be defined where amino acids within a group exchange preferentially with each other, and therefore resemble each other most in their impact on the overall protein structure (Schulz and Schirmer, supra). Examples of conservative amino acid substitutions include substitutions of amino acids within the sub-groups described above, for example, lysine for arginine and vice versa such that a positive charge may be maintained, glutamic acid for aspartic acid and vice versa such that a negative charge may be maintained, serine for threonine such that a free —OH can be maintained, and glutamine for asparagine such that a free —NH₂ can be maintained. “Semi-conservative mutations” include amino acid substitutions of amino acids within the same groups listed above, but not within the same sub-group. For example, the substitution of aspartic acid for asparagine, or asparagine for lysine, involves amino acids within the same group, but different sub-groups. “Non-conservative mutations” involve amino acid substitutions between different groups, for example, lysine for tryptophan, or phenylalanine for serine, etc.

In some embodiments, the nuclease comprises one or more amino acid substitutions and has an amino acid sequence having at least 70% identity (e.g., at least 75%, at least 80%, at least 85%, at least 90%, at least 93%, at least 95%, at least 98%, at least 99% identity, or 100% identity) to an amino acid sequence of SEQ ID NOs: 1-250. In some embodiments, the nuclease comprises one or more amino acid substitutions as compared to SEQ ID NOs: 1-250, and the one or more substitutions improved the editing efficiency of the nuclease.

The nucleases disclosed herein may be capable of recognizing a broad ranges of protospacer adjacent motifs (PAMs) which flank a target nucleic acid. In certain embodiments, the nuclease can only cleave a target nucleic acid if an appropriate PAM is present. In certain embodiments, the nuclease has broad ability for recognition of target nucleic acids, e.g., those lacking a PAM or broad PAM recognition.

A PAM is generally in proximity to a target sequence. For example, the PAM may be a sequence immediately or directly adjacent to the target nucleic acid. A PAM can be 5′ or 3′ of a target sequence. A PAM can be upstream or downstream of a target sequence. In one embodiment, the target nucleic acid is immediately flanked on 3′ end by a PAM. In one embodiment, the target nucleic acid is immediately flanked on 5′ end by a PAM.

A PAM can be 1, 2, 3, 4, 5, 6, 7, 8, 9, 10 or more nucleotides in length. In certain embodiments, a PAM is between 2-6 nucleotides in length.

Non-limiting examples of the PAM sequences include: CC, CA, AG, GT, TA, AC, CA, GC, CG, GG, CT, TG, GA, AGG, TGG, T-rich PAMs (such as TTT, TTG, TTC, etc.), NGG, NGA, NAG, NGGNG and NNAGAAW, NNNNGATT, NAAR (R=A or G), NNGRR (R=A or G), NNAGAA and NAAAAC, where “N” is any nucleotide.

In some embodiments, the nucleases disclosed herein are capable of recognizing a protospacer adjacent motif (PAM) sequence selected from the group comprising ATTA, GTTA, ATTG, GTTG, TTTA, TTTG, CTTA, and CTTG. In some embodiments, the PAM sequence comprises DTTR, wherein D is A, G, or T and R is A or G.

Different PAM sequences may confer different preferences and efficiencies for nuclease cleavage or modification by a desired nuclease. In some embodiments, the nuclease preferentially modifies a first target nucleic acid comprising PAM sequence ATTA as compared to a target nucleic acid comprising PAM sequence TTTR, wherein R is A or G. In some embodiments, higher efficiency of modification of the target nucleic acid by the nucleases disclosed herein are observed compared to the efficiency of modification by nuclease SEQ ID NO: 471. In some embodiments, higher efficiency of modification of a target nucleic acid by the nucleases disclosed herein are observed compared to the modification efficiency by nuclease SEQ ID NO: 471 when the target nucleic acid comprises PAM sequence is ATTA.

In some embodiments, the nuclease further comprises a nuclear localization sequence (NLS). The nuclear localization sequence may be appended, for example, to one or both of the N-terminus and C-terminus. In some embodiments, the nuclease comprises two or more NLSs. The two or more NLSs may be in tandem, separated by a linker, at either the N-terminus or C-terminus of the protein, or one or more may be internal to the open reading frame of the nuclease.

The nuclear localization sequence may comprise any amino acid sequence known in the art to functionally tag or direct a protein for import into a cell's nucleus (e.g., for nuclear transport). Usually, a nuclear localization sequence comprises one or more positively charged amino acids, such as lysine and arginine.

In some embodiments, the NLS is a monopartite sequence. A monopartite NLS comprises a single cluster of positively charged or basic amino acids. In some embodiments, the monopartite NLS comprises a sequence of K-K/R-X-K/R, wherein X can be any amino acid. Exemplary monopartite NLS sequences include those from the SV40 large T-antigen, c-Myc, and TUS-proteins. In select embodiments, the NLS comprises the NLS of SV40 large T-antigen, comprising an amino acid sequence of PKKKRKV (SEQ ID NO: 504).

In some embodiments, the NLS is a bipartite sequence. Bipartite NLSs comprise two clusters of basic amino acids, separated by a spacer of about 9-12 amino acids. Exemplary bipartite NLSs include the nuclear localization sequences of nucleoplasmin, EGL-12, or bipartite SV40. In select embodiments, the NLS comprises the NLS of nucleoplasmin, KR[PAATKKAGQA]KKKK (SEQ ID NO: 505).

In some embodiments, the two or more NLSs may have the same or different sequences. For example, in some embodiments, the nuclease comprises two NLSs, one sequence from the SV40 large T-antigen and one from nucleoplasmin.

The NLS may be appended to the nuclease by a linker. The linker may be a polypeptide of any amino acid sequence and length. The linker may act as a spacer peptide. In some embodiments, the linker is flexible. In some embodiments, the linker comprises at least one glycine and at least one serine. In some embodiments, the linker comprises an amino acid sequence consisting of (Gly₂Ser)ₙ, where n is the number of repeats comprising an integer from 2-20.

In some embodiments, the nuclease may comprise a tag (e.g., 3×FLAG tag, an HA tag, a Myc tag, and the like). The tag may facilitate tracking, separation, or purification of the nuclease. In some embodiments, the tag may be adjacent, either upstream or downstream, to a nuclear localization sequence. The tag may be at the N-terminus, a C-terminus, or a combination thereof of the nuclease.

In some embodiments, the nuclease is covalently attached to a peptide or protein in a fusion protein. The nuclease may be part of a fusion protein comprising another protein or protein domain. For example, the nuclease may be fused to another protein or protein domain that provides for tagging or visualization (e.g., GFP). The nuclease may be fused to a protein or protein domain that has another functionality or activity useful to target to certain DNA sequences (e.g., nuclease activity such as that provide by FokI nuclease, protein modification activity such as histone modification activity including acetylation or deacetylation or demethylation or methyltransferase activity, transcription modulation activity such as activity of a transcriptional activator or repressor, base editing activity such as deaminase activity, DNA modifying activity such as DNA methylation activity, and the like).

In some embodiments, the nuclease may be fused with one or more (e.g., two, three, four, or more) protein transduction domains or PTDs, also known as a CPP-cell penetrating peptide. A protein transduction domains is a polypeptide, polynucleotide, carbohydrate, or organic or inorganic compound that facilitates traversing a lipid bilayer, micelle, cell membrane, organelle membrane, or vesicle membrane. A PTD attached to another molecule facilitates the molecule traversing a membrane, for example going from extracellular space to intracellular space, or cytosol to within an organelle. In some embodiments, a PTD is covalently linked to a terminus of the nuclease (e.g., N-terminus, C-terminus, or both). In some embodiments, the PTD is inserted internally at a suitable insertion site. Examples of PTDs include but are not limited to a minimal undecapeptide protein transduction domain (corresponding to residues 47-57 of HIV-1 TAT comprising); a polyarginine sequence comprising a number of arginines sufficient to direct entry into a cell (e.g., 3, 4, 5, 6, 7, 8, 9, 10, or 10-50 arginines); a VP22 domain (Zender et al. (2002) Cancer Gene Ther. 9(6):489-96); a Drosophila Antennapedia protein transduction domain (Noguchi et al. (2003) Diabetes 52(7):1732-1737); a truncated human calcitonin peptide (Trehin et al. (2004) Pharm. Research 21:1248-1256); polylysine (Wender et al. (2000) Proc. Natl. Acad. Sci. USA 97:13003-13008); Transportan, and the like.

The nuclease may be fused via a linker polypeptide. The linker polypeptide may have any of a variety of amino acid sequences. Proteins can be joined by a spacer peptide, generally of a flexible nature, although other chemical linkages are not excluded. Suitable linkers include polypeptides of between 4 amino acids and 40 amino acids in length, or between 4 amino acids and 25 amino acids in length. These linkers can be produced by using synthetic, linker-encoding oligonucleotides to couple the proteins, or can be encoded by a nucleic acid sequence encoding the fusion protein. Peptide linkers with a degree of flexibility can be used. The linking peptides may have virtually any amino acid sequence, bearing in mind that the preferred linkers will have a sequence that results in a generally flexible peptide. The use of small amino acids, such as glycine and alanine, are of use in creating a flexible peptide. The creation of such sequences is routine to those of skill in the art. A variety of different linkers are commercially available and are considered suitable for use, including but not limited to, glycine-serine polymers, glycine-alanine polymers, and alanine-serine polymers.

### Compositions and Systems

Also disclosed herein are compositions comprising a nuclease as described herein or a nucleic acid molecule comprising a sequence encoding the nuclease.

Further disclosed herein are systems for modifying a target nucleic acid comprising a nuclease as described herein (e.g., a nuclease comprising an amino acid sequence having at least 70% identity to an amino acid sequence of SEQ ID NOs: 1-250 (e.g., at least 75%, at least 80%, at least 85%, at least 90%, at least 93%, at least 95%, at least 98%, at least 99% identity or 100% identity to an amino acid sequence of SEQ ID NOs: 1-250)) or a nucleic acid molecule comprising a sequence encoding the nuclease.

In some embodiments, the components of the system may be in the form of a composition. In some embodiments, the components of the present compositions or systems may be mixed, individually or in any combination, with a carrier which are also within the scope of the present disclosure. Exemplary carriers include buffers, antioxidants, preservatives, carbohydrates, surfactants, and the like.

Also disclosed is a cell comprising the compositions or systems described herein. In some embodiments, the cell is a prokaryotic cell. In some embodiments, the cell is a eukaryotic cell. In some embodiments, the cell is a mammalian cell. In some embodiments, the cell is a human cell.

The compositions or systems disclosed herein may further comprise at least one gRNA comprising a sequence complementary to at least a portion of a first target nucleic acid and a region that associates with the nuclease, or a nucleic acid encoding the at least one gRNA. In some embodiments, the at least one gRNA further comprises a sequence complementary to at least a portion of a second target nucleic acid. In instances when the composition or system comprises more than one gRNA, each may be encoded on the same or different nucleic acid as the other gRNA.

The gRNA may be a crRNA, crRNA/tracrRNA (or single guide RNA, sgRNA). The terms “gRNA,” “guide RNA” and “CRISPR guide sequence” may be used interchangeably throughout and refer to a nucleic acid comprising a sequence that associates with the nuclease and determines the sequence specificity of the nuclease. A gRNA may be engineered to hybridize to (e.g., be complementary to, partially or completely) a target nucleic acid sequence (e.g., the genome in a host cell).

In some embodiments, the at least one gRNA is encoded in a CRISPR RNA (crRNA) array. CRISPR arrays contain a series of direct repeats separated by short sequences called spacers. The nucleases described herein may have a preference for direct repeat sequences. For example, the CRISPR RNA (crRNA) may contain multiple gRNAs or may contain more than one different sequence each configured to hybridize a distinct target nucleic acid sequence.

The gRNA or portion thereof that hybridizes to the target nucleic acid (a target site) may be between 15-40 nucleotides in length. In some embodiments, the gRNA sequence that hybridizes to the target nucleic acid is 15, 16, 17, 18, 19, 20, 21, 22, 23, 24, 25, 26, 27, 28, 29, 30, 31, 32, 33, 34, 35, 36, 37, 38, 39, or 40 nucleotides in length. gRNAs or sgRNA(s) used in the present disclosure can be between about 5 and 100 nucleotides long, or longer (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 60, 61, 62, 63, 63, 64, 65, 66, 67, 68, 69, 70, 71, 72, 73, 74, 75, 76, 77, 78, 79, 80, 81, 82, 83, 84, 85, 86, 87, 88, 89, 90, 91 92, 93, 94, 95, 96, 97, 98, 99, or 100 nucleotides in length, or longer).

In addition to a sequence that binds to a target nucleic acid, in some embodiments, the gRNA may also comprise a scaffold sequence (e.g., tracrRNA). In some embodiments, such a chimeric gRNA may be referred to as a single guide RNA (sgRNA). Exemplary scaffold sequences will be evident to one of skill in the art and can be found, for example, in Jinek, et al. Science (2012) 337(6096):816-821, and Ran, et al. Nature Protocols (2013) 8:2281-2308, incorporated herein by reference in their entireties.

In some embodiments, the gRNA sequence does not comprise a scaffold sequence and a scaffold sequence is expressed as a separate transcript. In such embodiments, the gRNA sequence further comprises an additional sequence that is complementary to a portion of the scaffold sequence and functions to bind (hybridize) the scaffold sequence.

In some embodiments, the gRNA comprises a sequence of at least 50%, 55%, 60%, 65%, 70%, 75%, 80%, 85%, 90%, 95%, 96%, 97%, 98%, 99%, or at least 100% complementary to a target nucleic acid. In some embodiments, the sequence is at least 50%, 55%, 60%, 65%, 70%, 75%, 80%, 85%, 90%, 95%, 96%, 97%, 98%, 99%, or at least 100% complementary to the 3′ end of the target nucleic acid (e.g., the last 5, 6, 7, 8, 9, or 10 nucleotides of 3′ end of the target nucleic acid).

In some embodiments, the gRNA comprises a sequence with at least 60%, 70%, 80%, 85%, 90%, 95%, 96%, 97%, 98%, 99% identity or 100% identity to any one of SEQ ID NOs: 251-422 and 472-482. In some embodiments, the at least one gRNA comprises any one or more of SEQ ID NOs: 251-343. In some embodiments, the at least one gRNA comprises any one or more of SEQ ID NOs: 344-422. In some embodiments, the at least one gRNA comprises any one or more of SEQ ID NOs: 472-482.

gRNAs of the present disclosure may comprise a sequences having one or more nucleotide substitutions or mutations, truncations, or insertions relative to any of SEQ ID NOs: 251-343. The nucleotide substitutions or mutations, truncations, or insertions may increase stability, modify secondary structure elements, increase binding efficiency to a cognate nuclease or target strand, increase In some embodiments, the at least one gRNA comprises any one or more of SEQ ID NOs: 344-422. In some embodiments, the at least one gRNA comprises any one or more of SEQ ID NOs: 472-482. In some embodiments, the gRNA comprises SEQ ID NO: 346. In some embodiments, the gRNA comprises SEQ ID NO: 420. In some embodiments, the gRNA comprises SEQ ID NO: 481. In some embodiments, the gRNA comprises SEQ ID NO: 479.

In some embodiments, the gRNA comprises a spacer sequence. The spacer sequence may be of any length or sequence. In some embodiments, the spacer sequence is at least 18 (e.g., 18, 19, 20, 21, 22, 23, 24, etc.) nucleotides in length. In some embodiments, the spacer sequence is between 18 and 20 nucleotides in length. Thus, in certain embodiments, the spacer sequence is 18 nucleotides in length. In certain embodiments, the spacer sequence is 19 nucleotides in length. In certain embodiments, the spacer sequence is 20 nucleotides in length.

In some embodiments, the gRNA comprises a spacer sequence complementary to a first strand sequence of the target nucleic acid. In some embodiments, the first strand sequence is directly adjacent to a protospacer adjacent motif (PAM) sequence selected from the group comprising ATTA, GTTA, ATTG, GTTG, TTTA, TTTG, CTTA, and CTTG.

In some embodiments, the nuclease comprises SEQ ID NO: 21, and the at least one gRNA comprises a sequence with at least 60%, 70%, 80%, 85%, 90%, 95%, 96%, 97%, 98%, 99% identity or 100% identity to any one of SEQ ID NOs: 310, 344-349, 361-366, 404-422 and 479-482. In some embodiments, the nuclease comprises a sequence having at least 70%, 80%, 85%, 90%, 95%, 96%, 97%, 98%, or 99% identity to SEQ ID NO: 21, and wherein the at least one gRNA comprises any one of SEQ ID NOs: 310, 344-349, 361-366, 404-422 and 479-482. In some embodiments, the nuclease comprises SEQ ID NO: 21 or a sequence having at least 70%, 80%, 85%, 90%, 95%, 96%, 97%, 98%, or 99% identity to SEQ ID NO: 21, and the gRNA comprises SEQ ID NO: 346 or a sequence with at least 60%, 70%, 80%, 85%, 90%, 95%, 96%, 97%, 98%, 99% identity or 100% identity to SEQ ID NO: 346.

In some embodiments, the nuclease comprises SEQ ID NO: 24, and the at least one gRNA comprises a sequence with at least 60%, 70%, 80%, 85%, 90%, 95%, 96%, 97%, 98%, 99% identity or 100% identity to any one of SEQ ID NOs: 310, 313, 325, 346, 350-355, 358, 361-363, 367-372, and 389-392. In some embodiments, the nuclease comprises a sequence having at least 70%, 80%, 85%, 90%, 95%, 96%, 97%, 98%, or 99% identity to SEQ ID NO: 24, and wherein the at least one gRNA comprises any one of SEQ ID NOs: 310, 313, 325, 346, 350-355, 358, 361-363, 367-372, and 389-392. In some embodiments, the nuclease comprises a sequence having at least 70%, 80%, 85%, 90%, 95%, 96%, 97%, 98%, or 99% identity to SEQ ID NO: 24, and wherein the at least one gRNA comprises any one of SEQ ID NOs: 346, 352, 358, 361, 362, 368, 369, and 392. In some embodiments, the nuclease comprises SEQ ID NO: 24 or a sequence having at least 70%, 80%, 85%, 90%, 95%, 96%, 97%, 98%, or 99% identity to SEQ ID NO: 24, and the gRNA comprises SEQ ID NO: 346 or a sequence with at least 60%, 70%, 80%, 85%, 90%, 95%, 96%, 97%, 98%, 99% identity or 100% identity to SEQ ID NO: 346. In some embodiments, the nuclease comprises SEQ ID NO: 24 or a sequence having at least 70%, 80%, 85%, 90%, 95%, 96%, 97%, 98%, or 99% identity to SEQ ID NO: 24, and the gRNA comprises SEQ ID NO: 352 or a sequence with at least 60%, 70%, 80%, 85%, 90%, 95%, 96%, 97%, 98%, 99% identity or 100% identity to SEQ ID NO: 352.

In some embodiments, the nuclease comprises SEQ ID NO:36, and the at least one gRNA comprises a sequence with at least 60%, 70%, 80%, 85%, 90%, 95%, 96%, 97%, 98%, 99% identity or 100% identity to any one of SEQ ID NOs: 310, 313, 325, 346, 356-360, and 373-378. In some embodiments, the nuclease comprises a sequence having at least 70%, 80%, 85%, 90%, 95%, 96%, 97%, 98%, or 99% identity to SEQ ID NO: 36, and wherein the at least one gRNA comprises any one of SEQ ID NOs: 310, 313, 325, 346, 356-360, and 373-378. In some embodiments, the nuclease comprises SEQ ID NO: 36 or a sequence having at least 70%, 80%, 85%, 90%, 95%, 96%, 97%, 98%, or 99% identity to SEQ ID NO: 36, and the gRNA comprises SEQ ID NO: 346 or a sequence with at least 60%, 70%, 80%, 85%, 90%, 95%, 96%, 97%, 98%, 99% identity or 100% identity to SEQ ID NO: 346. In some embodiments, the nuclease comprises SEQ ID NO: 36 or a sequence having at least 70%, 80%, 85%, 90%, 95%, 96%, 97%, 98%, or 99% identity to SEQ ID NO: 36, and the gRNA comprises SEQ ID NO: 358 or a sequence with at least 60%, 70%, 80%, 85%, 90%, 95%, 96%, 97%, 98%, 99% identity or 100% identity to SEQ ID NO: 358.

In some embodiments, the nuclease comprises SEQ ID NO: 1, and the at least one gRNA comprises a sequence with at least 60%, 70%, 80%, 85%, 90%, 95%, 96%, 97%, 98%, 99% identity or 100% identity to any one of SEQ ID NOs: 251-256. In some embodiments, the nuclease comprises a sequence with at least having at least 70%, 80%, 85%, 90%, 95%, 96%, 97%, 98%, or 99% identity to SEQ ID NO: 1, and wherein the at least one gRNA comprises any one of SEQ ID NOs: 251-256.

In some embodiments, the nuclease comprises SEQ ID NO: 2, and the at least one gRNA comprises a sequence with at least 60%, 70%, 80%, 85%, 90%, 95%, 96%, 97%, 98%, 99% identity or 100% identity to any one of SEQ ID NOs: 257-259. In some embodiments, the nuclease comprises a sequence with at least having at least 70%, 80%, 85%, 90%, 95%, 96%, 97%, 98%, or 99% identity to SEQ ID NO: 2, and wherein the at least one gRNA comprises any one of SEQ ID NOs: 257-259.

In some embodiments, the nuclease comprises SEQ ID NO:3, and the at least one gRNA comprises a sequence with at least 60%, 70%, 80%, 85%, 90%, 95%, 96%, 97%, 98%, 99% identity or 100% identity to any one of SEQ ID NOs: 260-262. In some embodiments, the nuclease comprises a sequence with at least having at least 70%, 80%, 85%, 90%, 95%, 96%, 97%, 98%, or 99% identity to SEQ ID NO: 3, and wherein the at least one gRNA comprises any one of SEQ ID NOs: 260-262.

In some embodiments, the nuclease comprises SEQ ID NO:4, and the at least one gRNA comprises a sequence with at least 60%, 70%, 80%, 85%, 90%, 95%, 96%, 97%, 98%, 99% identity or 100% identity to any one of SEQ ID NOs: 263-265. In some embodiments, the nuclease comprises a sequence with at least having at least 70%, 80%, 85%, 90%, 95%, 96%, 97%, 98%, or 99% identity to SEQ ID NO: 4, and wherein the at least one gRNA comprises any one of SEQ ID NOs: 263-265.

In some embodiments, the nuclease comprises SEQ ID NO:5, and the at least one gRNA comprises a sequence with at least 60%, 70%, 80%, 85%, 90%, 95%, 96%, 97%, 98%, 99% identity or 100% identity to any one of SEQ ID NOs: 266-268. In some embodiments, the nuclease comprises a sequence with at least having at least 70%, 80%, 85%, 90%, 95%, 96%, 97%, 98%, or 99% identity to SEQ ID NO: 5, and wherein the at least one gRNA comprises any one of SEQ ID NOs: 266-268.

In some embodiments, the nuclease comprises SEQ ID NO:6, and the at least one gRNA comprises a sequence with at least 60%, 70%, 80%, 85%, 90%, 95%, 96%, 97%, 98%, 99% identity or 100% identity to any one of SEQ ID NOs: 269-271. In some embodiments, the nuclease comprises a sequence with at least having at least 70%, 80%, 85%, 90%, 95%, 96%, 97%, 98%, or 99% identity to SEQ ID NO: 6, and wherein the at least one gRNA comprises any one of SEQ ID NOs: 269-271.

In some embodiments, the nuclease comprises SEQ ID NO:7, and the at least one gRNA comprises a sequence with at least 60%, 70%, 80%, 85%, 90%, 95%, 96%, 97%, 98%, 99% identity or 100% identity to any one of SEQ ID NOs: 272-274. In some embodiments, the nuclease comprises a sequence with at least having at least 70%, 80%, 85%, 90%, 95%, 96%, 97%, 98%, or 99% identity to SEQ ID NO: 7, and wherein the at least one gRNA comprises any one of SEQ ID NOs: 272-274.

In some embodiments, the nuclease comprises SEQ ID NO:8, and the at least one gRNA comprises a sequence with at least 60%, 70%, 80%, 85%, 90%, 95%, 96%, 97%, 98%, 99% identity or 100% identity to any one of SEQ ID NOs: 275-277. In some embodiments, the nuclease comprises a sequence with at least having at least 70%, 80%, 85%, 90%, 95%, 96%, 97%, 98%, or 99% identity to SEQ ID NO: 8, and wherein the at least one gRNA comprises any one of SEQ ID NOs: 275-277.

In some embodiments, the nuclease comprises SEQ ID NO: 9, and the at least one gRNA comprises a sequence with at least 60%, 70%, 80%, 85%, 90%, 95%, 96%, 97%, 98%, 99% identity or 100% identity to any one of SEQ ID NOs: 278-280. In some embodiments, the nuclease comprises a sequence with at least having at least 70%, 80%, 85%, 90%, 95%, 96%, 97%, 98%, or 99% identity to SEQ ID NO: 9, and wherein the at least one gRNA comprises any one of SEQ ID NOs: 278-280.

In some embodiments, the nuclease comprises SEQ ID NO: 10, and the at least one gRNA comprises a sequence with at least 60%, 70%, 80%, 85%, 90%, 95%, 96%, 97%, 98%, 99% identity or 100% identity to any one of SEQ ID NOs: 281-283. In some embodiments, the nuclease comprises a sequence with at least having at least 70%, 80%, 85%, 90%, 95%, 96%, 97%, 98%, or 99% identity to SEQ ID NO: 10, and wherein the at least one gRNA comprises any one of SEQ ID NOs: 281-283.

In some embodiments, the nuclease comprises SEQ ID NO:11, and the at least one gRNA comprises a sequence with at least 60%, 70%, 80%, 85%, 90%, 95%, 96%, 97%, 98%, 99% identity or 100% identity to any one of SEQ ID NOs: 284-286. In some embodiments, the nuclease comprises a sequence with at least having at least 70%, 80%, 85%, 90%, 95%, 96%, 97%, 98%, or 99% identity to SEQ ID NO: 11, and wherein the at least one gRNA comprises any one of SEQ ID NOs: 284-286.

In some embodiments, the nuclease comprises SEQ ID NO: 12, and the at least one gRNA comprises a sequence with at least 60%, 70%, 80%, 85%, 90%, 95%, 96%, 97%, 98%, 99% identity or 100% identity to any one of SEQ ID NOs: 287-289. In some embodiments, the nuclease comprises a sequence with at least having at least 70%, 80%, 85%, 90%, 95%, 96%, 97%, 98%, or 99% identity to SEQ ID NO: 12, and wherein the at least one gRNA comprises any one of SEQ ID NOs: 287-289.

In some embodiments, the nuclease comprises SEQ ID NO: 13, and the at least one gRNA comprises a sequence with at least 60%, 70%, 80%, 85%, 90%, 95%, 96%, 97%, 98%, 99% identity or 100% identity to any one of SEQ ID NOs: 290-292. In some embodiments, the nuclease comprises a sequence with at least having at least 70%, 80%, 85%, 90%, 95%, 96%, 97%, 98%, or 99% identity to SEQ ID NO: 13, and wherein the at least one gRNA comprises any one of SEQ ID NOs: 290-292.

In some embodiments, the nuclease comprises SEQ ID NO: 14, and the at least one gRNA comprises a sequence with at least 60%, 70%, 80%, 85%, 90%, 95%, 96%, 97%, 98%, 99% identity or 100% identity to any one of SEQ ID NOs: 293-295. In some embodiments, the nuclease comprises a sequence with at least having at least 70%, 80%, 85%, 90%, 95%, 96%, 97%, 98%, or 99% identity to SEQ ID NO: 14, and wherein the at least one gRNA comprises any one of SEQ ID NOs: 293-295.

In some embodiments, the nuclease comprises SEQ ID NO:15, and the at least one gRNA comprises a sequence with at least 60%, 70%, 80%, 85%, 90%, 95%, 96%, 97%, 98%, 99% identity or 100% identity to any one of SEQ ID NOs: 296-298. In some embodiments, the nuclease comprises a sequence with at least having at least 70%, 80%, 85%, 90%, 95%, 96%, 97%, 98%, or 99% identity to SEQ ID NO: 15, and wherein the at least one gRNA comprises any one of SEQ ID NOs: 296-298.

In some embodiments, the nuclease comprises SEQ ID NO: 16, and the at least one gRNA comprises a sequence with at least 60%, 70%, 80%, 85%, 90%, 95%, 96%, 97%, 98%, 99% identity or 100% identity to any one of SEQ ID NOs: 299-301. In some embodiments, the nuclease comprises a sequence with at least having at least 70%, 80%, 85%, 90%, 95%, 96%, 97%, 98%, or 99% identity to SEQ ID NO: 16, and wherein the at least one gRNA comprises any one of SEQ ID NOs: 299-301.

In some embodiments, the nuclease comprises SEQ ID NO: 17, and the at least one gRNA comprises a sequence with at least 60%, 70%, 80%, 85%, 90%, 95%, 96%, 97%, 98%, 99% identity or 100% identity to any one of SEQ ID NOs: 302-304. In some embodiments, the nuclease comprises a sequence with at least having at least 70%, 80%, 85%, 90%, 95%, 96%, 97%, 98%, or 99% identity to SEQ ID NO: 17, and wherein the at least one gRNA comprises any one of SEQ ID NOs: 302-304.

In some embodiments, the nuclease comprises SEQ ID NO: 18, and the at least one gRNA comprises a sequence with at least 60%, 70%, 80%, 85%, 90%, 95%, 96%, 97%, 98%, 99% identity or 100% identity to any one of SEQ ID NOs: 305-307. In some embodiments, the nuclease comprises a sequence with at least having at least 70%, 80%, 85%, 90%, 95%, 96%, 97%, 98%, or 99% identity to SEQ ID NO: 18, and wherein the at least one gRNA comprises any one of SEQ ID NOs: 305-307.

In some embodiments, the nuclease comprises SEQ ID NO:19, and the at least one gRNA comprises a sequence with at least 60%, 70%, 80%, 85%, 90%, 95%, 96%, 97%, 98%, 99% identity or 100% identity to any one of SEQ ID NO: 308 or 379. In some embodiments, the nuclease comprises a sequence with at least having at least 70%, 80%, 85%, 90%, 95%, 96%, 97%, 98%, or 99% identity to SEQ ID NO: 19, and wherein the at least one gRNA comprises any one of SEQ ID NO: 308 or 379.

In some embodiments, the nuclease comprises SEQ ID NO: 20, and the at least one gRNA comprises a sequence with at least 60%, 70%, 80%, 85%, 90%, 95%, 96%, 97%, 98%, 99% identity or 100% identity to any one of SEQ ID NOs: 309, 346, 352, 358, 362-364, 380, 392-395, 410-420, 472-479, and 481. In some embodiments, the nuclease comprises a sequence having at least 70%, 80%, 85%, 90%, 95%, 96%, 97%, 98%, or 99% identity to SEQ ID NO: 20, and wherein the at least one gRNA comprises any one of SEQ ID NOs: 309, 346, 352, 358, 362-364, 380, 392-395, 410-420, 472-479, and 481. In some embodiments, the nuclease comprises a sequence having at least 70%, 80%, 85%, 90%, 95%, 96%, 97%, 98%, or 99% identity to SEQ ID NO: 20, and wherein the at least one gRNA comprises any one of SEQ ID NOs: 352, 358, 363, 364, 380, 392, and 417, or any one of SEQ ID NOs: 346 and 362, or any one of SEQ ID NOs: 410-419. In some embodiments, the nuclease comprises SEQ ID NO: 20 or a sequence having at least 70%, 80%, 85%, 90%, 95%, 96%, 97%, 98%, or 99% identity to SEQ ID NO: 20, and the gRNA comprises SEQ ID NO: 346 or a sequence with at least 60%, 70%, 80%, 85%, 90%, 95%, 96%, 97%, 98%, 99% identity or 100% identity to SEQ ID NO: 346.

In some embodiments, the nuclease comprises SEQ ID NO: 22, and the at least one gRNA comprises a sequence with at least 60%, 70%, 80%, 85%, 90%, 95%, 96%, 97%, 98%, 99% identity or 100% identity to any one of SEQ ID NOs: 311, 346, 381, and 398-399. In some embodiments, the nuclease comprises a sequence having at least 70%, 80%, 85%, 90%, 95%, 96%, 97%, 98%, or 99% identity to SEQ ID NO: 22, and wherein the at least one gRNA comprises any one of SEQ ID NOs: 311, 346, 381, and 398-399. In some embodiments, the nuclease comprises SEQ ID NO: 22 or a sequence having at least 70%, 80%, 85%, 90%, 95%, 96%, 97%, 98%, or 99% identity to SEQ ID NO: 22, and the gRNA comprises SEQ ID NO: 346 or a sequence with at least 60%, 70%, 80%, 85%, 90%, 95%, 96%, 97%, 98%, 99% identity or 100% identity to SEQ ID NO: 346.

In some embodiments, the nuclease comprises SEQ ID NO: 23, and the at least one gRNA comprises a sequence with at least 60%, 70%, 80%, 85%, 90%, 95%, 96%, 97%, 98%, 99% identity or 100% identity to any one of SEQ ID NOs: 312, 346, and 382. In some embodiments, the nuclease comprises a sequence having at least 70%, 80%, 85%, 90%, 95%, 96%, 97%, 98%, or 99% identity to SEQ ID NO: 23, and wherein the at least one gRNA comprises any one of SEQ ID NOs: 312, 346, and 382. In some embodiments, the nuclease comprises SEQ ID NO: 23 or a sequence having at least 70%, 80%, 85%, 90%, 95%, 96%, 97%, 98%, or 99% identity to SEQ ID NO: 23, and the gRNA comprises SEQ ID NO: 346 or a sequence with at least 60%, 70%, 80%, 85%, 90%, 95%, 96%, 97%, 98%, 99% identity or 100% identity to SEQ ID NO: 346.

In some embodiments, the nuclease comprises SEQ ID NO: 25, and the at least one gRNA comprises a sequence with at least 60%, 70%, 80%, 85%, 90%, 95%, 96%, 97%, 98%, 99% identity or 100% identity to any one of SEQ ID NOs: 314, 346, 383, and 400. In some embodiments, the nuclease comprises a sequence having at least 70%, 80%, 85%, 90%, 95%, 96%, 97%, 98%, or 99% identity to SEQ ID NO: 25, and wherein the at least one gRNA comprises any one of SEQ ID NOs: 314, 346, 383, and 400. In some embodiments, the nuclease comprises SEQ ID NO: 25 or a sequence having at least 70%, 80%, 85%, 90%, 95%, 96%, 97%, 98%, or 99% identity to SEQ ID NO: 25, and the gRNA comprises SEQ ID NO: 346 or a sequence with at least 60%, 70%, 80%, 85%, 90%, 95%, 96%, 97%, 98%, 99% identity or 100% identity to SEQ ID NO: 346.

In some embodiments, the nuclease comprises SEQ ID NO: 26, and the at least one gRNA comprises a sequence with at least 60%, 70%, 80%, 85%, 90%, 95%, 96%, 97%, 98%, 99% identity or 100% identity to any one of SEQ ID NOs: 315, 346, 384, 392, 396-397, 420, 479, and 481. In some embodiments, the nuclease comprises a sequence having at least 70%, 80%, 85%, 90%, 95%, 96%, 97%, 98%, or 99% identity to SEQ ID NO: 26, and wherein the at least one gRNA comprises any one of SEQ ID NOs: 315, 346, 384, 392, 396-397, 420, 479, and 481. In some embodiments, the nuclease comprises a sequence having at least 70%, 80%, 85%, 90%, 95%, 96%, 97%, 98%, or 99% identity to SEQ ID NO: 26, and wherein the at least one gRNA comprises any one of SEQ ID NOs: 346, 384 and 392.

In some embodiments, the nuclease comprises SEQ ID NO: 26 or a sequence having at least 70%, 80%, 85%, 90%, 95%, 96%, 97%, 98%, or 99% identity to SEQ ID NO: 26, and the gRNA comprises SEQ ID NO: 346 or a sequence with at least 60%, 70%, 80%, 85%, 90%, 95%, 96%, 97%, 98%, 99% identity or 100% identity to SEQ ID NO: 346.

In some embodiments, the nuclease comprises SEQ ID NO: 27, and the at least one gRNA comprises a sequence with at least 60%, 70%, 80%, 85%, 90%, 95%, 96%, 97%, 98%, 99% identity or 100% identity to any one of SEQ ID NOs: 316, 346, 385, and 401. In some embodiments, the nuclease comprises a sequence having at least 70%, 80%, 85%, 90%, 95%, 96%, 97%, 98%, or 99% identity to SEQ ID NO: 27, and wherein the at least one gRNA comprises any one of SEQ ID NOs: 316, 346, 385, and 401. In some embodiments, the nuclease comprises SEQ ID NO: 27 or a sequence having at least 70%, 80%, 85%, 90%, 95%, 96%, 97%, 98%, or 99% identity to SEQ ID NO: 27, and the gRNA comprises SEQ ID NO: 346 or a sequence with at least 60%, 70%, 80%, 85%, 90%, 95%, 96%, 97%, 98%, 99% identity or 100% identity to SEQ ID NO: 346.

In some embodiments, the nuclease comprises SEQ ID NO: 28, and the at least one gRNA comprises a sequence with at least 60%, 70%, 80%, 85%, 90%, 95%, 96%, 97%, 98%, 99% identity or 100% identity to any one of SEQ ID NOs: 317, 346, 386, and 402. In some embodiments, the nuclease comprises a sequence having at least 70%, 80%, 85%, 90%, 95%, 96%, 97%, 98%, or 99% identity to SEQ ID NO: 28, and wherein the at least one gRNA comprises any one of SEQ ID NOs: 317, 346, 386, and 402. In some embodiments, the nuclease comprises SEQ ID NO: 28 or a sequence having at least 70%, 80%, 85%, 90%, 95%, 96%, 97%, 98%, or 99% identity to SEQ ID NO: 28, and the gRNA comprises SEQ ID NO: 346 or a sequence with at least 60%, 70%, 80%, 85%, 90%, 95%, 96%, 97%, 98%, 99% identity or 100% identity to SEQ ID NO: 346.

In some embodiments, the nuclease comprises SEQ ID NO: 29, and the at least one gRNA comprises a sequence with at least 60%, 70%, 80%, 85%, 90%, 95%, 96%, 97%, 98%, 99% identity or 100% identity to any one of SEQ ID NOs: 318, 346, 387, and 403. In some embodiments, the nuclease comprises a sequence having at least 70%, 80%, 85%, 90%, 95%, 96%, 97%, 98%, or 99% identity to SEQ ID NO: 29, and wherein the at least one gRNA comprises any one of SEQ ID NOs: 318, 346, 387, and 403. In some embodiments, the nuclease comprises SEQ ID NO: 29 or a sequence having at least 70%, 80%, 85%, 90%, 95%, 96%, 97%, 98%, or 99% identity to SEQ ID NO: 29, and the gRNA comprises SEQ ID NO: 346 or a sequence with at least 60%, 70%, 80%, 85%, 90%, 95%, 96%, 97%, 98%, 99% identity or 100% identity to SEQ ID NO: 346.

In some embodiments, the nuclease comprises SEQ ID NO: 30, and the at least one gRNA comprises a sequence with at least 60%, 70%, 80%, 85%, 90%, 95%, 96%, 97%, 98%, 99% identity or 100% identity to SEQ ID NO: 319. In some embodiments, the nuclease comprises a sequence with at least having at least 70%, 80%, 85%, 90%, 95%, 96%, 97%, 98%, or 99% identity to SEQ ID NO: 30, and wherein the at least one gRNA comprises SEQ ID NO: 319.

In some embodiments, the nuclease comprises SEQ ID NO: 31, and the at least one gRNA comprises a sequence with at least 60%, 70%, 80%, 85%, 90%, 95%, 96%, 97%, 98%, 99% identity or 100% identity to SEQ ID NO: 320. In some embodiments, the nuclease comprises a sequence with at least having at least 70%, 80%, 85%, 90%, 95%, 96%, 97%, 98%, or 99% identity to SEQ ID NO: 31, and wherein the at least one gRNA comprises SEQ ID NO: 320.

In some embodiments, the nuclease comprises SEQ ID NO: 32, and the at least one gRNA comprises a sequence with at least 60%, 70%, 80%, 85%, 90%, 95%, 96%, 97%, 98%, 99% identity or 100% identity to SEQ ID NO: 321. In some embodiments, the nuclease comprises a sequence with at least having at least 70%, 80%, 85%, 90%, 95%, 96%, 97%, 98%, or 99% identity to SEQ ID NO: 32, and wherein the at least one gRNA comprises SEQ ID NO: 321.

In some embodiments, the nuclease comprises SEQ ID NO: 33, and the at least one gRNA comprises a sequence with at least 60%, 70%, 80%, 85%, 90%, 95%, 96%, 97%, 98%, 99% identity or 100% identity to SEQ ID NO: 322. In some embodiments, the nuclease comprises a sequence with at least having at least 70%, 80%, 85%, 90%, 95%, 96%, 97%, 98%, or 99% identity to SEQ ID NO: 33, and wherein the at least one gRNA comprises SEQ ID NO: 322.

In some embodiments, the nuclease comprises SEQ ID NO: 34, and the at least one gRNA comprises a sequence with at least 60%, 70%, 80%, 85%, 90%, 95%, 96%, 97%, 98%, 99% identity or 100% identity to any one of SEQ ID NO: 323 or 388. In some embodiments, the nuclease comprises a sequence with at least having at least 70%, 80%, 85%, 90%, 95%, 96%, 97%, 98%, or 99% identity to SEQ ID NO: 34, and wherein the at least one gRNA comprises any one of SEQ ID NO: 323 or 388.

In some embodiments, the nuclease comprises SEQ ID NO: 35, and the at least one gRNA comprises a sequence with at least 60%, 70%, 80%, 85%, 90%, 95%, 96%, 97%, 98%, 99% identity or 100% identity to SEQ ID NO: 324. In some embodiments, the nuclease comprises a sequence with at least having at least 70%, 80%, 85%, 90%, 95%, 96%, 97%, 98%, or 99% identity to SEQ ID NO: 35, and wherein the at least one gRNA comprises SEQ ID NO: 324.

In some embodiments, the nuclease comprises SEQ ID NO: 37, and the at least one gRNA comprises a sequence with at least 60%, 70%, 80%, 85%, 90%, 95%, 96%, 97%, 98%, 99% identity or 100% identity to SEQ ID NO: 326. In some embodiments, the nuclease comprises a sequence with at least having at least 70%, 80%, 85%, 90%, 95%, 96%, 97%, 98%, or 99% identity to SEQ ID NO: 37, and wherein the at least one gRNA comprises SEQ ID NO: 326.

In some embodiments, the nuclease comprises SEQ ID NO: 38, and the at least one gRNA comprises a sequence with at least 60%, 70%, 80%, 85%, 90%, 95%, 96%, 97%, 98%, 99% identity or 100% identity to SEQ ID NO: 327. In some embodiments, the nuclease comprises a sequence with at least having at least 70%, 80%, 85%, 90%, 95%, 96%, 97%, 98%, or 99% identity to SEQ ID NO: 38, and wherein the at least one gRNA comprises SEQ ID NO: 327.

In some embodiments, the nuclease comprises SEQ ID NO: 39, and the at least one gRNA comprises a sequence with at least 60%, 70%, 80%, 85%, 90%, 95%, 96%, 97%, 98%, 99% identity or 100% identity to SEQ ID NO: 328. In some embodiments, the nuclease comprises a sequence with at least having at least 70%, 80%, 85%, 90%, 95%, 96%, 97%, 98%, or 99% identity to SEQ ID NO: 39, and wherein the at least one gRNA comprises SEQ ID NO: 328.

In some embodiments, the nuclease comprises SEQ ID NO: 40, and the at least one gRNA comprises a sequence with at least 60%, 70%, 80%, 85%, 90%, 95%, 96%, 97%, 98%, 99% identity or 100% identity to SEQ ID NO: 329. In some embodiments, the nuclease comprises a sequence with at least having at least 70%, 80%, 85%, 90%, 95%, 96%, 97%, 98%, or 99% identity to SEQ ID NO: 40, and wherein the at least one gRNA comprises SEQ ID NO: 329.

In some embodiments, the nuclease comprises SEQ ID NO: 41, and the at least one gRNA comprises a sequence with at least 60%, 70%, 80%, 85%, 90%, 95%, 96%, 97%, 98%, 99% identity or 100% identity to SEQ ID NO: 330. In some embodiments, the nuclease comprises a sequence with at least having at least 70%, 80%, 85%, 90%, 95%, 96%, 97%, 98%, or 99% identity to SEQ ID NO: 41, and wherein the at least one gRNA comprises SEQ ID NO: 330.

In some embodiments, the nuclease comprises SEQ ID NO: 42, and the at least one gRNA comprises a sequence with at least 60%, 70%, 80%, 85%, 90%, 95%, 96%, 97%, 98%, 99% identity or 100% identity to SEQ ID NO: 331. In some embodiments, the nuclease comprises a sequence with at least having at least 70%, 80%, 85%, 90%, 95%, 96%, 97%, 98%, or 99% identity to SEQ ID NO: 42, and wherein the at least one gRNA comprises SEQ ID NO: 331.

In some embodiments, the nuclease comprises SEQ ID NO: 43, and the at least one gRNA comprises a sequence with at least 60%, 70%, 80%, 85%, 90%, 95%, 96%, 97%, 98%, 99% identity or 100% identity to SEQ ID NO: 332. In some embodiments, the nuclease comprises a sequence with at least having at least 70%, 80%, 85%, 90%, 95%, 96%, 97%, 98%, or 99% identity to SEQ ID NO: 43, and wherein the at least one gRNA comprises SEQ ID NO: 332.

In some embodiments, the nuclease comprises SEQ ID NO: 44, and the at least one gRNA comprises a sequence with at least 60%, 70%, 80%, 85%, 90%, 95%, 96%, 97%, 98%, 99% identity or 100% identity to SEQ ID NO: 333. In some embodiments, the nuclease comprises a sequence with at least having at least 70%, 80%, 85%, 90%, 95%, 96%, 97%, 98%, or 99% identity to SEQ ID NO: 44, and wherein the at least one gRNA comprises SEQ ID NO: 333.

In some embodiments, the nuclease comprises SEQ ID NO: 45, and the at least one gRNA comprises a sequence with at least 60%, 70%, 80%, 85%, 90%, 95%, 96%, 97%, 98%, 99% identity or 100% identity to SEQ ID NO: 334. In some embodiments, the nuclease comprises a sequence with at least having at least 70%, 80%, 85%, 90%, 95%, 96%, 97%, 98%, or 99% identity to SEQ ID NO: 45, and wherein the at least one gRNA comprises SEQ ID NO: 334.

In some embodiments, the nuclease comprises SEQ ID NO: 46, and the at least one gRNA comprises a sequence with at least 60%, 70%, 80%, 85%, 90%, 95%, 96%, 97%, 98%, 99% identity or 100% identity to SEQ ID NO: 335. In some embodiments, the nuclease comprises a sequence with at least having at least 70%, 80%, 85%, 90%, 95%, 96%, 97%, 98%, or 99% identity to SEQ ID NO: 46, and wherein the at least one gRNA comprises SEQ ID NO: 335.

In some embodiments, the nuclease comprises SEQ ID NO: 47, and the at least one gRNA comprises a sequence with at least 60%, 70%, 80%, 85%, 90%, 95%, 96%, 97%, 98%, 99% identity or 100% identity to SEQ ID NO: 336. In some embodiments, the nuclease comprises a sequence with at least having at least 70%, 80%, 85%, 90%, 95%, 96%, 97%, 98%, or 99% identity to SEQ ID NO: 47, and wherein the at least one gRNA comprises SEQ ID NO: 336.

In some embodiments, the nuclease comprises SEQ ID NO: 48, and the at least one gRNA comprises a sequence with at least 60%, 70%, 80%, 85%, 90%, 95%, 96%, 97%, 98%, 99% identity or 100% identity to SEQ ID NO: 337. In some embodiments, the nuclease comprises a sequence with at least having at least 70%, 80%, 85%, 90%, 95%, 96%, 97%, 98%, or 99% identity to SEQ ID NO: 48, and wherein the at least one gRNA comprises SEQ ID NO: 337.

In some embodiments, the nuclease comprises SEQ ID NO: 49, and the at least one gRNA comprises a sequence with at least 60%, 70%, 80%, 85%, 90%, 95%, 96%, 97%, 98%, 99% identity or 100% identity to SEQ ID NO: 338. In some embodiments, the nuclease comprises a sequence with at least having at least 70%, 80%, 85%, 90%, 95%, 96%, 97%, 98%, or 99% identity to SEQ ID NO: 49, and wherein the at least one gRNA comprises SEQ ID NO: 338.

In some embodiments, the nuclease comprises SEQ ID NO: 50, and the at least one gRNA comprises a sequence with at least 60%, 70%, 80%, 85%, 90%, 95%, 96%, 97%, 98%, 99% identity or 100% identity to SEQ ID NO: 339. In some embodiments, the nuclease comprises a sequence with at least having at least 70%, 80%, 85%, 90%, 95%, 96%, 97%, 98%, or 99% identity to SEQ ID NO: 50, and wherein the at least one gRNA comprises SEQ ID NO: 339.

In some embodiments, the nuclease comprises SEQ ID NO: 51, and the at least one gRNA comprises a sequence with at least 60%, 70%, 80%, 85%, 90%, 95%, 96%, 97%, 98%, 99% identity or 100% identity to SEQ ID NO: 340. In some embodiments, the nuclease comprises a sequence with at least having at least 70%, 80%, 85%, 90%, 95%, 96%, 97%, 98%, or 99% identity to SEQ ID NO: 51, and wherein the at least one gRNA comprises SEQ ID NO: 340.

In some embodiments, the nuclease comprises SEQ ID NO: 52, and the at least one gRNA comprises a sequence with at least 60%, 70%, 80%, 85%, 90%, 95%, 96%, 97%, 98%, 99% identity or 100% identity to SEQ ID NO: 341. In some embodiments, the nuclease comprises a sequence with at least having at least 70%, 80%, 85%, 90%, 95%, 96%, 97%, 98%, or 99% identity to SEQ ID NO: 52, and wherein the at least one gRNA comprises SEQ ID NO: 341.

In some embodiments, the nuclease comprises SEQ ID NO: 53, and the at least one gRNA comprises a sequence with at least 60%, 70%, 80%, 85%, 90%, 95%, 96%, 97%, 98%, 99% identity or 100% identity to SEQ ID NO: 342. In some embodiments, the nuclease comprises a sequence with at least having at least 70%, 80%, 85%, 90%, 95%, 96%, 97%, 98%, or 99% identity to SEQ ID NO: 53, and wherein the at least one gRNA comprises SEQ ID NO: 342.

In some embodiments, the nuclease comprises SEQ ID NO: 54, and the at least one gRNA comprises a sequence with at least 60%, 70%, 80%, 85%, 90%, 95%, 96%, 97%, 98%, 99% identity or 100% identity to SEQ ID NO: 343. In some embodiments, the nuclease comprises a sequence with at least having at least 70%, 80%, 85%, 90%, 95%, 96%, 97%, 98%, or 99% identity to SEQ ID NO: 54, and wherein the at least one gRNA comprises SEQ ID NO: 343.

In some embodiments, the nuclease comprises any of SEQ ID NOs: 1-19 and 30-54 or a sequence having at least 70%, 80%, 85%, 90%, 95%, 96%, 97%, 98%, or 99% identity to any of SEQ ID NOs: 1-19 and 30-54, and the gRNA comprises SEQ ID NO: 346 or a sequence with at least 60%, 70%, 80%, 85%, 90%, 95%, 96%, 97%, 98%, 99% identity or 100% identity to SEQ ID NO: 346.

In some embodiments, the gRNAs described herein may comprise one or more nucleotide substitutions or mutations (e.g., 1, 2, 3, 4, 5, 6, 7, 8, 9, 10, 15, 20, etc.) relative to any of SEQ ID NOs: 251-343.

In some embodiments, the gRNAs comprise one or more truncations or deletions of one or more nucleotides relative to any of SEQ ID NOs: 251-343. The truncations or deletions may be at one or both of the 3′ and 5′ ends of the sequence, or within or internal to the sequence related to any of SEQ ID NOs: 251-343. The truncations or deletions may encompass a single nucleotide or may comprise deletion or truncation of a series of two or more consecutive nucleotides (e.g., 2, 3, 4, 5, 10, 15, 20, etc.). In some embodiments, the gRNAs of the present invention may comprise a truncation sequence corresponding to or estimated to be the crRNA:tracrRNA stem.

In some embodiments, the gRNA comprises a tracr sequence. The gRNA may comprise one or more sequence deletions in or near the region encompassing the tracr sequence. For example, the one or more sequence deletions may comprise sequences predicted to form a stem-loop structure. In some embodiments, the one or more sequence deletions comprises sequences predicted to form a stem-loop structure at or near 5′ end of the gRNA. In some embodiments, the gRNA comprises SEQ ID NO: 346. In some embodiments, the gRNA comprises SEQ ID NO: 420. In some embodiments, the gRNA comprises SEQ ID NO: 481. In some embodiments, the gRNA comprises SEQ ID NO: 479.

In some embodiments, the gRNAs comprise one or more insertion or additions of one or more nucleotides relative to any of SEQ ID NOs: 251-343. The insertion or additions may be at one or both of 3′ and 5′ ends of the sequence, or within the sequence related to any of SEQ ID NOs: 251-343. The insertion or additions may encompass a single nucleotide or may comprise deletion or truncation of a series of two or more consecutive nucleotides (e.g., 2, 3, 4, 5, 10, 15, 20, etc.). In some embodiments, the gRNAs of the present invention may comprise an artificial stem-loop between crRNA & tracrRNA.

The gRNA may be a non-naturally occurring gRNA.

In certain embodiments, engineering the nucleases for use in eukaryotic cells may involve codon-optimization. It will be appreciated that changing native codons to those most frequently used in mammals allows for maximum expression of the system proteins in mammalian cells (e.g., human cells). Such modified nucleic acid sequences are commonly described in the art as “codon-optimized,” or as utilizing “mammalian-preferred” or “human-preferred” codons. In some embodiments, the nucleic acid sequence is considered codon-optimized if at least about 60% (e.g., 65%, 70%, 75%, 80%, 85%, 90%, 95%, or 98%) of the codons encoded therein are mammalian preferred codons.

In some cases, the compositions or systems disclosed herein may further comprise a donor polynucleotide. For example, in applications in which it is desirable to insert a polynucleotide sequence into the genome where a target sequence is cleaved, a donor polynucleotide (a nucleic acid comprising a donor sequence) can also be provided to the cell. By a “donor sequence” or “donor polynucleotide” or “donor template” it is meant a nucleic acid sequence to be inserted at the site targeted by the nuclease (e.g., after dsDNA cleavage, after nicking a target DNA, after dual nicking a target DNA, and the like). In some cases, the donor sequence is provided to the cell as single-stranded DNA. In some cases, the donor template is provided to the cell as double-stranded DNA. It may be introduced into a cell in linear or circular form. If introduced in linear form, the ends of the donor sequence may be protected (e.g., from exonucleolytic degradation) by any convenient method and such methods are known to those of skill in the art. For example, one or more dideoxynucleotide residues can be added to the 3′ terminus of a linear molecule and/or self-complementary oligonucleotides can be ligated to one or both ends. A donor template can be introduced into a cell as part of a vector molecule having additional sequences such as, for example, replication origins, promoters and genes encoding antibiotic resistance. Moreover, donor template can be introduced as naked nucleic acid, as nucleic acid complexed with an agent such as a liposome or poloxamer, or can be delivered by viruses (e.g., adenovirus, AAV).

The present disclosure also provides for one or more nucleic acids encoding the nucleases and gRNA disclosed herein, vectors containing these nucleic acids and cells containing the vectors. The vectors may be used to propagate the segment in an appropriate cell and/or to allow expression from the segment (e.g., an expression vector). The person of ordinary skill in the art would be aware of the various vectors available for propagation and expression of a nucleic acid sequence.

In some embodiments, the one or more nucleic acids comprise one or more messenger RNAs, one or more vectors, or any combination thereof. In some embodiments, the one or more nucleic acids includes a messenger RNA for expression of the nuclease and at least one nucleic acid provides the gRNA. A single nucleic acid may encode the nuclease and the at least one gRNA, or the nuclease can be encoded on a separate nucleic acid from the at least one gRNA.

In some embodiments, the nuclease is provided as a split-nuclease (e.g., a nuclease can in some cases be delivered as a split-nuclease, or a nucleic acid(s) encoding a split-nuclease) such that two separate proteins together form a functional nuclease. In some such cases the sequences that encode the two parts of the split-nuclease protein are present on the same vector. In some cases, they are present on separate vectors, e.g., as part of a vector system that encodes the nucleases, the gRNA(s), and systems thereof.

The present disclosure further provides engineered, non-naturally occurring vectors and vector systems, which can encode one or more or all of the components of the present system. The vector(s) can be introduced into a cell that is capable of expressing the polypeptide encoded thereby, including any suitable prokaryotic or eukaryotic cell.

The vectors of the present disclosure can be delivered to a eukaryotic cell in a subject, such as a mammalian subject, such as a human subject. Modification of the eukaryotic cells via the present system can take place in a cell culture.

Viral and non-viral based gene transfer methods can be used to introduce nucleic acids encoding components of the present system into cells, tissues, or a subject. Such methods can be used to administer nucleic acids encoding components of the present system to cells in culture, or in a host organism. Non-viral vector delivery systems include DNA plasmids, cosmids, RNA (e.g., a transcript of a vector described herein), a nucleic acid, and a nucleic acid complexed with a delivery vehicle. Viral vector delivery systems include DNA and RNA viruses, which have either episomal or integrated genomes after delivery to the cell. Viral vectors include, for example, retroviral, lentiviral, adenoviral, adeno-associated and herpes simplex viral vectors.

In certain embodiments, plasmids that are non-replicative, or plasmids that can be cured by high temperature may be used, such that any or all of the necessary components of the composition or system may be removed from the cells under certain conditions. For example. this may allow for DNA integration by transforming bacteria of interest, but then being left with engineered strains that have no memory of the plasmids or vectors used for the integration.

A variety of viral constructs can be used to deliver the present composition or system (such as a nuclease and one or more gRNA(s)) to the targeted cells and/or a subject. Nonlimiting examples of such recombinant viruses include recombinant adeno-associated virus (AAV), recombinant adenoviruses, recombinant lentiviruses, recombinant retroviruses, recombinant herpes simplex viruses, recombinant poxviruses, phages, etc. The present disclosure provides vectors capable of integration in the host genome, such as retrovirus or lentivirus. See, e.g., Ausubel et al., Current Protocols in Molecular Biology, John Wiley & Sons, New York, 1989; Kay, M. A., et al., 2001 Nat. Medic. 7(1):33-40; and Walther W. and Stein U., 2000 Drugs, 60(2): 249-71, incorporated herein by reference.

In one embodiment, a DNA segment encoding the nuclease is contained in a plasmid vector that allows expression of the protein and subsequent isolation and purification of the protein produced by the recombinant vector. Accordingly, the nucleases disclosed herein can be purified following expression, obtained by chemical synthesis, or obtained by recombinant methods.

To construct cells that express the present system, expression vectors for stable or transient expression of the system, or any of its components, may be constructed via methods as described herein or known in the art and introduced into cells. For example, nucleic acids encoding the components of the present system may be cloned into a suitable expression vector, such as a plasmid or a viral vector in operable linkage to a suitable promoter. The selection of expression vectors/plasmids/viral vectors should be suitable for integration and replication in eukaryotic cells. In some embodiments, a single nucleic acid comprises a first promoter operatively linked to a nuclease and a second promoter operatively linked to a gRNA. In some cases, the single nucleic acid is a vector.

In certain embodiments, one or more promoters can drive the expression of one or more sequences (e.g., the nuclease and/or the gRNA) in prokaryotic cells. Promoters that may be used include T7 RNA polymerase promoters, constitutive E. coli promoters, and promoters that could be broadly recognized by transcriptional machinery in a wide range of bacterial organisms. The composition or system may be used with various bacterial hosts.

In certain embodiments, one or more promoters can drive the expression of one or more sequences (e.g., the nuclease and/or the gRNA) in mammalian cells, such as when comprised in a mammalian expression vector. Examples of mammalian expression vectors include pCDM8 (Seed, Nature (1987) 329:840, incorporated herein by reference) and pMT2PC (Kaufman, et al., EMBO J. (1987) 6:187, incorporated herein by reference). When used in mammalian cells, the expression vector's control functions are typically provided by one or more regulatory elements. For example, commonly used promoters are derived from polyoma, adenovirus 2, cytomegalovirus, simian virus 40, and others disclosed herein and known in the art. For other suitable expression systems for both prokaryotic and eukaryotic cells see, e.g., Chapters 16 and 17 of Sambrook, et al., MOLECULAR CLONING: A LABORATORY MANUAL. 2nd eds., Cold Spring Harbor Laboratory, Cold Spring Harbor Laboratory Press, Cold Spring Harbor, N. Y., 1989, incorporated herein by reference.

Promoters for use in expressing the nucleases and gRNAs herein may comprise any of a number of promoters known to the art, wherein the promoter is constitutive, regulatable or inducible, cell type specific, tissue-specific, or species specific. In addition to the sequence sufficient to direct transcription, a promoter sequence of the invention can also include sequences of other regulatory elements that are involved in modulating transcription (e.g., enhancers, Kozak sequences and introns). Many promoter/regulatory sequences useful for driving constitutive expression of a gene are available in the art and include, but are not limited to, for example, CMV (cytomegalovirus promoter), EF1a (human elongation factor 1 alpha promoter), SV40 (simian vacuolating virus 40 promoter), PGK (mammalian phosphoglycerate kinase promoter), Ube (human ubiquitin C promoter), human beta-actin promoter, rodent beta-actin promoter, CBh (chicken beta-actin promoter), CAG (hybrid promoter contains CMV enhancer, chicken beta actin promoter, and rabbit beta-globin splice acceptor), TRE (Tetracycline response element promoter), H1 (human polymerase III RNA promoter), U6 (human U6 small nuclear promoter), and the like. Additional promoters that can be used for expression of the components of the present system, include, without limitation, cytomegalovirus (CMV) intermediate early promoter, a viral LTR such as the Rous sarcoma virus LTR, HIV-LTR, HTLV-1 LTR, Maloney murine leukemia virus (MMLV) LTR, myeloproliferative sarcoma virus (MPSV) LTR, spleen focus-forming virus (SFFV) LTR, the simian virus 40 (SV40) early promoter, herpes simplex tk virus promoter, elongation factor 1-alpha (EF1-α) promoter with or without the EF1-α intron. Additional promoters include any constitutively active promoter. Alternatively, any regulatable promoter may be used, such that its expression can be modulated within a cell. In embodiments, a polymerase II promoter is used to drive expression of the nuclease (e.g., a CMV promoter) and a polymerase III promoter (e.g., U6 promoter) is used to drive expression of the gRNA.

Different promoters and regulatory elements may be used to achieve proper balance (expression level ratio) between the components of the systems (e.g., the nuclease, the at least one gRNA). For example, in some cases a nucleic acid includes a promoters and regulatory elements that is operably linked to (and therefore regulates/modulates translation of) a sequence encoding the nuclease. In some cases, a subject nucleic acid includes a promoters and regulatory elements that is operably linked to a sequence encoding the gRNA. In some cases, the sequence encoding the nuclease and the sequence encoding the gRNA are both operably linked to the same promoters and regulatory elements.

A variety of promoter types are suitable for use. A promoter can be a constitutively active promoter (e.g., a promoter that is constitutively in an active/“ON” state), it may be an inducible promoter (e.g., a promoter whose state, active/“ON” or inactive/“OFF”, is controlled by an external stimulus, e.g., the presence of a particular temperature, compound, or protein.), it may be a spatially restricted promoter (e.g., tissue specific promoter, cell type specific promoter, etc.), and it may be a temporally restricted promoter (e.g., the promoter is in the “ON” state or “OFF” state during specific stages of embryonic development or during specific stages of a biological process, e.g., hair follicle cycle in mice).

Moreover, inducible and tissue specific expression of RNA or proteins can be accomplished by placing the nucleic acid encoding such a molecule under the control of an inducible or tissue specific promoter/regulatory sequence. Promoters may direct expression of the nucleic acid in a particular cell type (e.g., tissue-specific regulatory elements are used to express the nucleic acid). Such regulatory elements include promoters that may be tissue specific or cell specific. The term “tissue specific” as it applies to a promoter refers to a promoter that is capable of directing selective expression of a nucleotide sequence of interest to a specific type of tissue (e.g., seeds) in the relative absence of expression of the same nucleotide sequence of interest in a different type of tissue. The term “cell type specific” as applied to a promoter refers to a promoter that is capable of directing selective expression of a nucleotide sequence of interest in a specific type of cell in the relative absence of expression of the same nucleotide sequence of interest in a different type of cell within the same tissue. The term “cell type specific” when applied to a promoter also means a promoter capable of promoting selective expression of a nucleotide sequence of interest in a region within a single tissue. Cell type specificity of a promoter may be assessed using methods well known in the art, e.g., immunohistochemical staining.

Examples of tissue specific or inducible promoter/regulatory sequences which are useful for this purpose include, but are not limited to, the rhodopsin promoter, the MMTV LTR inducible promoter, the SV40 late enhancer/promoter, synapsin 1 promoter, ET hepatocyte promoter, GS glutamine synthase promoter and many others. Various commercially available ubiquitous as well as tissue-specific promoters and tumor-specific are available, for example from InvivoGen. In addition, promoters that are well known in the art can be induced in response to inducing agents such as metals, glucocorticoids, tetracycline, hormones, and the like, are also contemplated for use with the invention. Thus, it will be appreciated that the present disclosure includes the use of any promoter/regulatory sequence known in the art that is capable of driving expression of the desired nuclease or gRNA operably linked thereto.

Examples of spatially restricted promoters include, but are not limited to, neuron-specific promoters, adipocyte-specific promoters, cardiomyocyte-specific promoters, smooth muscle-specific promoters, photoreceptor-specific promoters, etc. Neuron-specific spatially restricted promoters include, but are not limited to, a neuron-specific enolase (NSE) promoter (see, e.g., EMBL HSENO2, X51956); an aromatic amino acid decarboxylase (AADC) promoter; a neurofilament promoter (see, e.g., GenBank HUMNFL, L04147); a synapsin promoter (see, e.g., GenBank HUMSYNIB, M55301); a thy-1 promoter; a serotonin receptor promoter (see, e.g., GenBank S62283); a tyrosine hydroxylase promoter (TH); a GnRH promoter; an L7 promoter; a DNMT promoter; an enkephalin; a myelin basic protein (MBP) promoter; a Ca2+-calmodulin-dependent protein kinase II-alpha (CamKIIα) promoter; a CMV enhancer/platelet-derived growth factor-β promoter; and the like. Suitable liver-specific promoters can in some cases include, but are not limited to: TTR, Albumin, and AAT promoters. Suitable CNS-specific promoters can in some cases include, but are not limited to: Synapsin 1, BM88, CHNRB2, GFAP, and CAMK2a promoters. Suitable muscle-specific promoters can in some cases include, but are not limited to: MYOD1, MYLK2, SPc5-12 (synthetic), α-MHC, MLC-2, MCK, MHCK7, human cardiac troponin C (cTnC) and desmin promoters. Adipocyte-specific spatially restricted promoters include, but are not limited to, aP2 gene promoter/enhancer, e.g., a region from −5.4 kb to +21 bp of a human aP2; a glucose transporter-4 (GLUT4); a fatty acid translocase (FAT/CD36) promoter; a stearoyl-CoA desaturase-1 (SCD1) promoter; a leptin promoter; an adiponectin promoter; an adipsin promoter; a resistin promoter; and the like. Cardiomyocyte-specific spatially restricted promoters include, but are not limited to control sequences derived from the following genes: myosin light chain-2, α-myosin heavy chain, AE3, cardiac troponin C, cardiac actin, and the like. Smooth muscle-specific spatially restricted promoters include, but are not limited to, an SM22α promoter; a smoothelin promoter; an α-smooth muscle actin promoter; and the like. For example, a 0.4 kb region of the SM22α promoter, within which lie two CArG elements, has been shown to mediate vascular smooth muscle cell-specific. Photoreceptor-specific spatially restricted promoters include, but are not limited to, a rhodopsin promoter; a rhodopsin kinase promoter; a beta phosphodiesterase gene; a retinitis pigmentosa gene promoter; an interphotoreceptor retinoid-binding protein (IRBP) gene enhancer; an IRBP gene promoter; and the like.

Examples of inducible promoters include, but are not limited to, heat shock promoter, tetracycline-regulated promoter, steroid-regulated promoter, metal-regulated promoter, estrogen receptor-regulated promoter, etc. Inducible promoters can therefore be regulated by molecules including, but not limited to, doxycycline; an estrogen receptor; an estrogen receptor fusion; an estrogen analog; IPTG; and the like. Inducible promoters suitable for use include any inducible promoter described herein or known to one of ordinary skill in the art. Examples of inducible promoters include, without limitation, chemically/biochemically-regulated and physically-regulated promoters such as alcohol-regulated promoters, tetracycline-regulated promoters (e.g., anhydrotetracycline (aTc)-responsive promoters and other tetracycline-responsive promoter systems, which include a tetracycline repressor protein (tetR), a tetracycline operator sequence (tetO) and a tetracycline transactivator fusion protein ((TA)), steroid-regulated promoters (e.g., promoters based on the rat glucocorticoid receptor, human estrogen receptor, moth ecdysone receptors, and promoters from the steroid/retinoid/thyroid receptor superfamily), metal-regulated promoters (e.g., promoters derived from metallothionein (proteins that bind and sequester metal ions) genes from yeast, mouse and human), pathogenesis-regulated promoters (e.g., induced by salicylic acid, ethylene or benzothiadiazole (BTH)), temperature/heat-inducible promoters (e.g., heat shock promoters), and light-regulated promoters (e.g., light responsive promoters from plant cells).

Inducible promoters include sugar-inducible promoters (e.g., lactose-inducible promoters; arabinose-inducible promoters); amino acid-inducible promoters; alcohol-inducible promoters; and the like. Suitable promoters include, e.g., lactose-regulated systems (e.g., lactose operon systems, sugar-regulated systems, isopropyl-beta-D-thiogalactopyranoside (IPTG) inducible systems, arabinose regulated systems (e.g., arabinose operon systems, e.g., an ARA operon promoter, pBAD, pARA, portions thereof, combinations thereof and the like), synthetic amino acid regulated systems, fructose repressors, a tac promoter/operator (pTac), tryptophan promoters, PhoA promoters, recA promoters, proU promoters, est-1 promoters, tetA promoters, cadA promoters, nar promoters, P_{L} promoters, espA promoters, and the like, or combinations thereof. In certain cases, a promoter comprises a Lac-Z, or portions thereof. In some cases, a promoter comprises a Lac operon, or portions thereof. In some cases, an inducible promoter comprises an ARA operon promoter, or portions thereof. In certain embodiments an inducible promoter comprises an arabinose promoter or portions thereof. An arabinose promoter can be obtained from any suitable bacteria. In some cases, an inducible promoter comprises an arabinose operon of E. coli or B. subtilis. In some cases, an inducible promoter is activated by the presence of a sugar or an analog thereof. Non-limiting examples of sugars and sugar analogs include lactose, arabinose (e.g., L-arabinose), glucose, sucrose, fructose, IPTG, and the like. Suitable promoters include a T7 promoter; a pBAD promoter; a lacIQ promoter; and the like. In some cases, the promoter is a J23119 promoter. Many bacterial promoters are known in the art; bacterial promoters can be found on the internet at parts(dot)igem(dot)org/promoters.

In some cases, the promoter is a reversible promoter. Suitable reversible promoters, including reversible inducible promoters are known in the art. Such reversible promoters may be isolated and derived from many organisms. Such reversible promoters may be isolated and derived from many organisms, e.g., eukaryotes and prokaryotes. Modification of reversible promoters derived from a first organism for use in a second organism is well known in the art. Modification of reversible promoters derived from a first organism for use in a second organism, e.g., a first prokaryote and a second a eukaryote, a first eukaryote and a second a prokaryote, etc., is well known in the art. Such reversible promoters, and systems based on such reversible promoters but also comprising additional control proteins, include, but are not limited to, alcohol regulated promoters (e.g., alcohol dehydrogenase I (alcA) gene promoter, promoters responsive to alcohol transactivator proteins (AlcR)), tetracycline regulated promoters, (e.g., promoter systems including TetActivators, TetON, TetOFF), steroid regulated promoters (e.g., rat glucocorticoid receptor promoter systems, human estrogen receptor promoter systems, retinoid promoter systems, thyroid promoter systems, ecdysone promoter systems, mifepristone promoter systems), metal regulated promoters (e.g., metallothionein promoter systems), pathogenesis-related regulated promoters (e.g., salicylic acid regulated promoters, ethylene regulated promoters, benzothiadiazole regulated promoters), temperature regulated promoters (e.g., heat shock inducible promoters (e.g., HSP-70, HSP-90, soybean heat shock promoter), light regulated promoters, synthetic inducible promoters, and the like.

Thus, it will be appreciated that the present disclosure includes the use of any promoter/regulatory sequence capable of driving expression of the desired nuclease or RNA operably linked thereto.

Additionally, the vector described herein for expression of the nucleases and/or gRNAs may contain, for example, some or all of the following: a selectable marker gene, such as the neomycin gene for selection of stable or transient transfectants in host cells; enhancer/promoter sequences from the immediate early gene of human CMV for high levels of transcription; transcription termination and RNA processing signals from SV40 for mRNA stability; 5′- and 3′-untranslated regions for mRNA stability and translation efficiency from highly-expressed genes like α-globin or β-globin; SV40 polyoma origins of replication and ColE1 for proper episomal replication; internal ribosome binding sites (IRESes), versatile multiple cloning sites; T7 and SP6 RNA promoters for in vitro transcription of sense and antisense RNA; a “suicide switch” or “suicide gene” which when triggered causes cells carrying the vector to die (e.g., HSV thymidine kinase, an inducible caspase such as iCasp9), and reporter gene for assessing expression of the chimeric receptor. Suitable vectors and methods for producing vectors containing transgenes are well known and available in the art. Selectable markers also include chloramphenicol resistance, tetracycline resistance, spectinomycin resistance, streptomycin resistance, erythromycin resistance, rifampicin resistance, bleomycin resistance, thermally adapted kanamycin resistance, gentamycin resistance, hygromycin resistance, trimethoprim resistance, dihydrofolate reductase (DHFR), GPT; the URA3, HIS4, LEU2, and TRP1 genes of S. cerevisiae.

When introduced into the cell, the vectors may be maintained as an autonomously replicating sequence or extrachromosomal element or may be integrated into host DNA.

The present compositions and systems (e.g., proteins, polynucleotides encoding these proteins, or compositions comprising the proteins and/or polynucleotides described herein) may be delivered by any suitable means. In certain embodiments, the composition or system is delivered in vivo. In other embodiments, the composition or system is delivered to isolated/cultured cells (e.g., autologous iPS cells) in vitro.

Vectors and nucleic acids according to the present disclosure can be transformed, transfected, or otherwise introduced into a wide variety of host cells. Transfection refers to the taking up of nucleic acid by a host cell whether or not any coding sequences are in fact expressed. Numerous methods of transfection are known to the ordinarily skilled artisan, for example, lipofectamine, calcium phosphate co-precipitation, electroporation, DEAE-dextran treatment, microinjection, viral infection, and other methods known in the art. Transduction refers to entry of a virus into the cell and expression (e.g., transcription and/or translation) of sequences delivered by the viral vector genome. In the case of a recombinant vector, “transduction” generally refers to entry of the recombinant viral vector into the cell and expression of a nucleic acid of interest delivered by the vector genome.

Any of the vectors comprising a nucleic acid sequence that encodes the components of the present compositions and system is also within the scope of the present disclosure. Such a vector may be delivered into host cells by a suitable method. Methods of delivering vectors to cells are well known in the art and may include DNA or RNA electroporation, transfection reagents such as liposomes or nanoparticles to delivery DNA or RNA, delivery of DNA, RNA, or protein by mechanical deformation, or viral transduction. In some embodiments, the vectors are delivered to host cells by viral transduction. Nucleic acids can be delivered as part of a larger construct, such as a plasmid or viral vector, or directly, e.g., by electroporation, lipid vesicles, viral transporters, microinjection, and biolistics (high-speed particle bombardment). Similarly, the construct containing the one or more transgenes can be delivered by any method appropriate for introducing nucleic acids into a cell.

Additionally, delivery vehicles such as nanoparticle- and lipid-based mRNA or protein delivery systems can be used. Further examples of delivery vehicles include lentiviral vectors, ribonucleoprotein (RNP) complexes, lipid-based delivery system, gene gun, hydrodynamic, electroporation or nucleofection microinjection, biolistics, and the like.

In some embodiments, the vector is a viral construct, e.g., a recombinant adeno-associated virus construct, a recombinant adenoviral construct, a recombinant lentiviral construct, a recombinant retroviral construct, etc. Suitable viral vectors include, but are not limited to, viral vectors based on vaccinia virus; poliovirus; adenovirus; adeno-associated virus; SV40; herpes simplex virus; human immunodeficiency virus; a retroviral vector (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus); and the like.

In some embodiments, the vector is an AAV vector. By adeno-associated virus, or “AAV” it is meant the virus itself or derivatives thereof. The term covers all subtypes and both naturally occurring and recombinant forms, except where required otherwise, for example, AAV type 1 (AAV-1), AAV type 2 (AAV-2), AAV type 3 (AAV-3), AAV type 4 (AAV-4), AAV type 5 (AAV-5), AAV type 6 (AAV-6), AAV type 7 (AAV-7), AAV type 8 (AAV-8), AAV type 9 (AAV-9), AAV type 10 (AAV-10), AAV type 11 (AAV-11), avian AAV, bovine AAV, canine AAV, equine AAV, primate AAV, non-primate AAV, ovine AAV, a hybrid AAV (i.e., an AAV comprising a capsid protein of one AAV subtype and genomic material of another subtype), an AAV comprising a mutant AAV capsid protein or a chimeric AAV capsid (i.e. a capsid protein with regions or domains or individual amino acids that are derived from two or more different serotypes of AAV, e.g. AAV-DJ, AAV-LK3, AAV-LK19). “Primate AAV” refers to AAV that infect primates, “non-primate AAV” refers to AAV that infect non-primate mammals, “bovine AAV” refers to AAV that infect bovine mammals, etc.

By a “recombinant AAV vector” or “rAAV vector” it is meant an AAV virus or AAV viral chromosomal material comprising a polynucleotide sequence not of AAV origin (e.g., a polynucleotide heterologous to AAV), typically a nucleic acid sequence of interest to be integrated into the cell following the subject methods. In general, the heterologous polynucleotide is flanked by at least one, and generally by two AAV inverted terminal repeat sequences (ITRs). In some instances, the recombinant viral vector also comprises viral genes important for the packaging of the recombinant viral vector material. Packaging refers to the series of intracellular events that result in the assembly and encapsulation of a viral particle, e.g., an AAV viral particle. Examples of nucleic acid sequences important for AAV packaging include the AAV “rep” and “cap” genes, which encode for replication and encapsulation proteins of adeno-associated virus, respectively. The term rAAV vector encompasses both rAAV vector particles and rAAV vector plasmids.

A “viral particle” refers to a single unit of virus comprising a capsid encapsulating a virus-based polynucleotide, e.g., the viral genome (as in a wild-type virus), or, e.g., the subject targeting vector (as in a recombinant virus). An AAV viral particle refers to a viral particle composed of at least one AAV capsid protein (typically by all of the capsid proteins of a wild-type AAV) and an encapsulated polynucleotide AAV vector. If the particle comprises a heterologous polynucleotide (e.g., a polynucleotide other than a wild-type AAV genome, such as a transgene to be delivered to a mammalian cell), it is typically referred to as an “rAAV vector particle” or simply an “rAAV vector.” Thus, production of rAAV particle necessarily includes production of rAAV vector, as such a vector is contained within an rAAV particle.

A rAAV virion can be constructed a variety of methods. For example, the heterologous sequence(s) can be directly inserted into an AAV genome which has had the major AAV open reading frames (“ORFs”) excised therefrom. Other portions of the AAV genome can also be deleted, so long as a sufficient portion of the ITRs remain to allow for replication and packaging functions. In order to produce rAAV virions, an AAV expression vector can be introduced into a suitable host cell using known techniques, such as by transfection. Particularly suitable transfection methods include calcium phosphate co-, direct micro-injection into cultured cells, electroporation, liposome mediated gene transfer, lipid-mediated transduction, and nucleic acid delivery using high-velocity microprojectiles. Suitable cells for producing rAAV virions include microorganisms, yeast cells, insect cells, and mammalian cells, that can be, or have been, used as recipients of a heterologous DNA molecule.

An AAV virus that is produced may be replication competent or replication-incompetent. A “replication-competent” virus (e.g., a replication-competent AAV) refers to a phenotypically wild-type virus that is infectious and is also capable of being replicated in an infected cell (e.g., in the presence of a helper virus or helper virus functions). In the case of AAV, replication competence generally requires the presence of functional AAV packaging genes. In general, rAAV vectors as described herein are replication-incompetent in mammalian cells (especially in human cells) by virtue of the lack of one or more AAV packaging genes. Typically, such rAAV vectors lack any AAV packaging gene sequences in order to minimize the possibility that replication competent AAV are generated by recombination between AAV packaging genes and an incoming rAAV vector.

Retroviruses, for example, lentiviruses, are suitable for use in methods of the present disclosure. Commonly used retroviral vectors are unable to produce viral proteins required for productive infection. Rather, replication of the vector requires growth in a packaging cell line. To generate viral particles comprising nucleic acids of interest, the retroviral nucleic acids comprising the nucleic acid are packaged into viral capsids by a packaging cell line. Different packaging cell lines provide a different envelope protein (ecotropic, amphotropic or xenotropic) to be incorporated into the capsid, this envelope protein determining the specificity of the viral particle for the cells (ecotropic for murine and rat; amphotropic for most mammalian cell types including human, dog, and mouse; and xenotropic for most mammalian cell types except murine cells). The appropriate packaging cell line may be used to ensure that the cells are targeted by the packaged viral particles. Methods of introducing subject vector expression vectors into packaging cell lines and of collecting the viral particles that are generated by the packaging lines are well known in the art. Nucleic acids can also introduced by direct micro-injection (e.g., injection of RNA).

As noted elsewhere herein, proteins may instead be provided to cells as RNA (e.g., an RNA comprising the translational control element as discussed elsewhere herein). Methods of introducing RNA into cells may include, for example, direct injection, transfection, or any other method used for the introduction of DNA. The nuclease may also be introduced into a host cell directly as protein. In such instances, the nuclease may be delivered as an RNP (ribonucleoprotein complex) in which it is already complexed with an appropriate guide RNA.

The disclosed nucleic acids (e.g., vectors) and proteins can be delivered to cells using any convenient method. Suitable methods include, e.g., viral infection (e.g., AAV, adenovirus, lentiviral), transfection, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct micro injection, nanoparticle-mediated nucleic acid delivery, and the like.

In some cases, a nuclease is delivered to a cell in a particle, or associated with a particle. In some cases, a nuclease is delivered with a cationic lipid and a hydrophilic polymer, for instance wherein the cationic lipid comprises 1,2-dioleoyl-3-trimethylammonium-propane (DOTAP) or 1,2-ditetradecanoyl-sn-glycero-3-phosphocholine (DMPC) and/or wherein the hydrophilic polymer comprises ethylene glycol or polyethylene glycol (PEG); and/or wherein the particle further comprises cholesterol.

A nuclease may be delivered using particles or lipid envelopes. For example, a biodegradable core-shell structured nanoparticle with a poly (β-amino ester) (PBAE) core enveloped by a phospholipid bilayer shell can be used. In some cases, particles/nanoparticles based on self-assembling bioadhesive polymers are used; such particles/nanoparticles may be applied to oral delivery of peptides, intravenous delivery of peptides and nasal delivery of peptides, e.g., to the brain. Other embodiments, such as oral absorption and ocular delivery of hydrophobic drugs are also contemplated. A molecular envelope technology, which involves an engineered polymer envelope which is protected and delivered to the desired cell, can be used.

Lipidoid compounds (e.g., as described in U.S. Patent Application Publication No. 2011/0293703) are also useful in the delivery of polynucleotides, and can be used to deliver the disclosed nucleases (or RNA or DNA encoding thereof). In one aspect, the aminoalcohol lipidoid compounds are combined with an agent to be delivered to a cell to form microparticles, nanoparticles, liposomes, or micelles. The aminoalcohol lipidoid compounds may be combined with other aminoalcohol lipidoid compounds, polymers (synthetic or natural), surfactants, cholesterol, carbohydrates, proteins, lipids, etc. to form the particles. These particles may then optionally be combined with a pharmaceutical excipient to form a pharmaceutical composition.

A poly(beta-amino alcohol) (PBAA) can be used to deliver a nuclease, or a nucleic acid encoding thereof, and gRNA, or a nucleic acid encoding thereof, to a target cell. U.S. Patent Application Publication No. 2013/0302401 relates to a class of poly(beta-amino alcohols) (PBAAs) that has been prepared using combinatorial polymerization.

Sugar-based particles, for example GalNAc, as described in International Patent Publication No. WO2014118272 (incorporated herein by reference in its entirety and Nair, J K et al., 2014, Journal of the American Chemical Society 136 (49), 16958-16961) can be used to deliver a nuclease, or a nucleic acid encoding thereof, and gRNA, or a nucleic acid encoding thereof, to a target cell.

In some cases, lipid nanoparticles (LNPs) are used to deliver a nuclease, or a nucleic acid encoding thereof, and gRNA, or a nucleic acid encoding thereof, to a target cell. Negatively charged polymers such as RNA may be loaded into LNPs at low pH values (e.g., pH 4) where the ionizable lipids display a positive charge. However, at physiological pH values, the LNPs exhibit a low surface charge compatible with longer circulation times. Four species of ionizable cationic lipids have been focused upon, namely 1,2-dilineoyl-3-dimethylammonium-propane (DLinDAP), 1,2-dilinoleyloxy-3-N,N-dimethylaminopropane (DLinDMA), 1,2-dilinoleyloxy-keto-N,N-dimethyl-3-aminopropane (DLinKDMA), and 1,2-dilinoleyl-4-(2-dimethylaminoethyl)-[1,3]-dioxolane (DLinKC2-DMA). Preparation of LNPs and is described in, e.g., Rosin et al. (2011) Molecular Therapy 19:1286-2200). The cationic lipids 1,2-dilineoyl-3-dimethylammonium-propane (DLinDAP), 1,2-dilinoleyloxy-3-N,N-dimethylaminopropane (DLinDMA), 1,2-dilinoleyloxyketo-N,N-dimethyl-3-aminopropane (DLinK-DMA), 1,2-dilinoleyl-4-(2-dimethylaminoethyl)-[1,3]-dioxolane (DLinKC2-DMA), (3-o-[2″-(methoxypolyethyleneglycol 2000) succinoyl]-1,2-dimyristoyl-sn-glycol (PEG-S-DMG), and R-3-[(.omega.-methoxy-poly(ethylene glycol) 2000) carbamoyl]-1,2-dimyristyloxlpropyl-3-amine (PEG-C-DOMG) may be used. A nucleic acid may be encapsulated in LNPs containing DLinDAP, DLinDMA, DLinK-DMA, and DLinKC2-DMA (cationic lipid:DSPC:CHOL:PEGS-DMG or PEG-C-DOMG at 40:10:40:10 molar ratios). In some cases, 0.2% SP-DiOC18 is incorporated.

Spherical Nucleic Acid (SNA™) constructs and other nanoparticles (particularly gold nanoparticles) can be used to deliver a nuclease, or a nucleic acid encoding thereof, and gRNA, or a nucleic acid encoding thereof, to a target cell.

Self-assembling nanoparticles with RNA may be constructed with polyethyleneimine (PEI) that is PEGylated with an Arg-Gly-Asp (RGD) peptide ligand attached at the distal end of the polyethylene glycol (PEG).

Nanoparticles suitable for use in delivering a nuclease, or a nucleic acid encoding thereof, and gRNA, or a nucleic acid encoding thereof, to a target cell may be provided in different forms, e.g., as solid nanoparticles (e.g., metal such as silver, gold, iron, titanium), non-metal, lipid-based solids, polymers), suspensions of nanoparticles, or combinations thereof. Metal, dielectric, and semiconductor nanoparticles may be prepared, as well as hybrid structures (e.g., core-shell nanoparticles). Nanoparticles made of semiconducting material may also be labeled quantum dots if they are small enough (typically below 10 nm) that quantization of electronic energy levels occurs. Such nanoscale particles are used in biomedical applications as drug carriers or imaging agents and may be adapted for similar purposes in the present disclosure. In general, a “nanoparticle” refers to any particle having a diameter of less than 1000 nm. In some cases, nanoparticles suitable for use in delivering a nuclease or nucleic acid to a target cell have a diameter of 500 nm or less, e.g., from 25 nm to 35 nm, from 35 nm to 50 nm, from 50 nm to 75 nm, from 75 nm to 100 nm, from 100 nm to 150 nm, from 150 nm to 200 nm, from 200 nm to 300 nm, from 300 nm to 400 nm, or from 400 nm to 500 nm. In some cases, nanoparticles suitable for use in delivering a nuclease or nucleic acid to a target cell have a diameter of from 25 nm to 200 nm.

In some cases, an exosome is used to deliver a nuclease, or a nucleic acid encoding thereof, and gRNA, or a nucleic acid encoding thereof, to a target cell. Exosomes are endogenous nano-vesicles that transport RNAs and proteins, and which can deliver RNA to the brain and other target organs.

In some cases, a liposome is used to deliver a nuclease, or a nucleic acid encoding thereof, and gRNA, or a nucleic acid encoding thereof, to a target cell. Liposomes are spherical vesicle structures composed of a uni- or multi-lamellar lipid bilayer surrounding internal aqueous compartments and a relatively impermeable outer lipophilic phospholipid bilayer. Liposomes can be made from several different types of lipids; however, phospholipids are most commonly used to generate liposomes. Although liposome formation is spontaneous when a lipid film is mixed with an aqueous solution, it can also be expedited by applying force in the form of shaking by using a homogenizer, sonicator, or an extrusion apparatus. Several other additives may be added to liposomes in order to modify their structure and properties. For instance, either cholesterol or sphingomyelin may be added to the liposomal mixture in order to help stabilize the liposomal structure and to prevent the leakage of the liposomal inner cargo. A liposome formulation may be mainly comprised of natural phospholipids and lipids such as 1,2-distearoryl-sn-glycero-3-phosphatidyl choline (DSPC), sphingomyelin, egg phosphatidylcholines and monosialoganglioside.

A stable nucleic-acid-lipid particle (SNALP) can be used to deliver a nuclease, or a nucleic acid encoding thereof, and gRNA, or a nucleic acid encoding thereof, to a target cell. The SNALP formulation may contain the lipids 3-N-[(methoxypoly(ethylene glycol) 2000) carbamoyl]-1,2-dimyristyloxy-propylamine (PEG-C-DMA), 1,2-dilinoleyloxy-N,N-dimethyl-3-aminopropane (DLinDMA), 1,2-distearoyl-sn-glycero-3-phosphocholine (DSPC) and cholesterol, in a 2:40:10:48 molar percent ratio. The SNALP liposomes may be prepared by formulating D-Lin-DMA and PEG-C-DMA with distearoylphosphatidylcholine (DSPC), Cholesterol and siRNA using a 25:1 lipid/siRNA ratio and a 48/40/10/2 molar ratio of Cholesterol/D-Lin-DMA/DSPC/PEG-C-DMA. The resulting SNALP liposomes can be about 80-100 nm in size. A SNALP may comprise synthetic cholesterol (Sigma-Aldrich, St Louis, Mo., USA), dipalmitoylphosphatidylcholine (Avanti Polar Lipids, Alabaster, Ala., USA), 3-N-[(w-methoxy poly(ethylene glycol) 2000) carbamoyl]-1,2-dimyrestyloxypropylamine, and cationic 1,2-dilinoleyloxy-3-N,Ndimethylaminopropane. A SNALP may comprise synthetic cholesterol (Sigma-Aldrich), 1,2-distearoyl-sn-glycero-3-phosphocholine (DSPC; Avanti Polar Lipids Inc.), PEG-CDMA, and 1,2-dilinoleyloxy-3-(N;N-dimethyl)aminopropane (DLinDMA).

Other cationic lipids, such as amino lipid 2,2-dilinoleyl-4-dimethylaminoethyl-[1,3]-dioxolane (DLin-KC2-DMA) can be used to deliver a nuclease or nucleic acid to a target cell. A preformed vesicle with the following lipid composition may be contemplated: amino lipid, distearoylphosphatidylcholine (DSPC), cholesterol and (R)-2,3-bis(octadecyloxy) propyl-1-(methoxy poly(ethylene glycol) 2000) propylcarbamate (PEG-lipid) in the molar ratio 40/10/40/10, respectively, and a FVII siRNA/total lipid ratio of approximately 0.05 (w/w). To ensure a narrow particle size distribution in the range of 70-90 nm and a low polydispersity index of 0.11.+−.0.04 (n=56), the particles may be extruded up to three times through 80 nm membranes prior to adding the guide RNA. Particles containing the highly potent amino lipid 16 may be used, in which the molar ratio of the four lipid components 16, DSPC, cholesterol and PEG-lipid (50/10/38.5/1.5) which may be further optimized to enhance in vivo activity.

Lipids may be formulated with a nuclease, or a nucleic acid encoding thereof, and gRNA, or a nucleic acid encoding thereof, to form lipid nanoparticles (LNPs). Suitable lipids include, but are not limited to, DLin-KC2-DMA4, C12-200 and colipids disteroylphosphatidyl choline, cholesterol, and PEG-DMG may be formulated with a nuclease or nucleic acid using a spontaneous vesicle formation procedure.

A nuclease, or a nucleic acid encoding thereof, and gRNA, or a nucleic acid encoding thereof, may be delivered encapsulated in PLGA microspheres such as those further described in US published applications 20130252281, 20130245107, and 20130244279.

Supercharged proteins can be used to deliver a nuclease, or a nucleic acid encoding thereof, and gRNA, or a nucleic acid encoding thereof, to a target cell. Supercharged proteins are a class of engineered or naturally occurring proteins with unusually high positive or negative net theoretical charge. Both supernegatively and superpositively charged proteins exhibit the ability to withstand thermally or chemically induced aggregation. Superpositively charged proteins are also able to penetrate mammalian cells. Associating cargo with these proteins, such as plasmid DNA, RNA, or other proteins, can facilitate the functional delivery of these macromolecules into mammalian cells both in vitro and in vivo.

Cell Penetrating Peptides (CPPs) can be used to deliver a nuclease, or a nucleic acid encoding thereof, and gRNA, or a nucleic acid encoding thereof, to a target cell. CPPs typically have an amino acid composition that either contains a high relative abundance of positively charged amino acids such as lysine or arginine or has sequences that contain an alternating pattern of polar/charged amino acids and non-polar, hydrophobic amino acids.

### Methods

The disclosure also provides methods of modifying a target nucleic acid sequence (e.g., DNA or RNA). The phrase “modifying a nucleic acid sequence,” as used herein, refers to modifying at least one physical feature of a nucleic acid sequence of interest. Nucleic acid modifications include, for example, single or double strand breaks, deletion, or insertion of one or more nucleotides, and other modifications that affect the structural integrity or nucleotide sequence of the nucleic acid sequence. The modifications may comprise one or more of modification of the target nucleic acid, modulation of transcription from the target nucleic acid, and modification of a polypeptide associated with a target nucleic acid. The methods comprise contacting a target nucleic acid sequence with a composition as disclosed herein, a system disclosed herein or a composition comprising the system.

In one embodiment, the method introduces a single strand or double strand break in the target nucleic acid sequence. In this respect, the disclosed systems may direct cleavage of one or both strands of a target DNA sequence, such as within the target genomic DNA sequence and/or within the complement of the target sequence.

In some embodiments, contacting a target nucleic acid sequence comprises introducing the composition or system described herein into the cell. As described above the composition or system may be introduced into eukaryotic or prokaryotic cells by methods known in the art.

The cell may be a prokaryotic cell, a plant cell, an insect cell, a vertebrate cell, an invertebrate cell, an animal cell, a mammalian cell, or a human cell. In some embodiments, the cell is a plant cell. In some embodiments, the cell is an insect cell. In some embodiments, the cell is a vertebrate cell. In some embodiments, the cell is an invertebrate cell. In some embodiments, the cell is a mammalian cell. In some embodiments, the cell is a human cell. In some cases, the cell is ex vivo (e.g., fresh isolate-early passage). In some cases, the cell is in vivo. In some cases, the cell is in culture in vitro (e.g., immortalized cell line).

Cells may be from established cell lines or they may be primary cells, where “primary cells,” “primary cell lines,” and “primary cultures” are used interchangeably herein to refer to cells and cells cultures that have been derived from a subject and allowed to grow in vitro for a limited number of passages of the culture. For example, primary cultures are cultures that may have been passaged 0 times, 1 time, 2 times, 4 times, 5 times, 10 times, or 15 times, but not enough times go through the crisis stage. Typically, the primary cell lines are maintained for fewer than 10 passages in culture.

Suitable cells include, but are not limited to: bacterial cell; an archaeal cell; a eukaryotic cell; a cell of a single-cell eukaryotic organism; a plant cell; a protozoa cell; an algal cell, e.g., Botryococcus braunii, Chlamydomonas reinhardtii, Nannochloropsis gaditana, Chlorella pyrenoidosa, Sargassum patens, C. agardh, and the like; a fungal cell (e.g., a yeast cell); an animal cell; a cell from an invertebrate animal (e.g. fruit fly, a cnidarian, an echinoderm, a nematode, etc.); a cell of an insect (e.g., a mosquito; a bee; an agricultural pest; etc.); a cell of an arachnid (e.g., a spider; a tick; etc.); a cell of a vertebrate animal (e.g., a fish, an amphibian, a reptile, a bird, a mammal); a cell of a mammal (e.g., a cell of a rodent; a cell of a human; a cell of a non-human mammal; a cell of a rodent (e.g., a mouse, a rat); a cell of a lagomorph (e.g., a rabbit); a cell of an ungulate (e.g., a cow, a horse, a camel, a llama, a vicuña, a sheep, a goat, etc.); a cell of a marine mammal (e.g., a whale, a seal, an elephant seal, a dolphin, a sea lion; etc.) and the like. Any type of cell may be of interest (e.g. a stem cell, e.g. an embryonic stem (ES) cell, an induced pluripotent stem (iPS) cell, a germ cell (e.g., an oocyte, a sperm, an oogonia, a spermatogonia, etc.), an adult stem cell, a somatic cell, e.g. a fibroblast, a hematopoietic cell, a neuron, a muscle cell, a bone cell, a hepatocyte, a pancreatic cell; an in vitro or in vivo embryonic cell of an embryo at any stage, e.g., a 1-cell, 2-cell, 4-cell, 8-cell, etc. stage zebrafish embryo; etc.). In some cases, the cell is a cell that does not originate from a natural organism (e.g., the cell can be a synthetically made cell; also referred to as an artificial cell).

Non-limiting examples of plant cell include cells from: plant crops, fruits, vegetables, grains, soybean, corn, maize, wheat, seeds, tomatoes, rice, cassava, sugarcane, pumpkin, hay, potatoes, cotton, cannabis, tobacco, flowering plants, conifers, gymnosperms, angiosperms, ferns, clubmosses, hornworts, liverworts, mosses, dicotyledons, monocotyledons, seaweeds (e.g., kelp), and the like.

Suitable cells include a stem cell (e.g., an embryonic stem (ES) cell, an induced pluripotent stem (iPS) cell; a germ cell (e.g., an oocyte, a sperm, an oogonia, a spermatogonia, etc.); a somatic cell, e.g., a fibroblast, an oligodendrocyte, a glial cell, a hematopoietic cell, a neuron, a muscle cell, a bone cell, a hepatocyte, a pancreatic cell, etc.

Suitable cells include human embryonic stem cells, fetal cardiomyocytes, myofibroblasts, mesenchymal stem cells, autotransplated expanded cardiomyocytes, adipocytes, totipotent cells, pluripotent cells, blood stem cells, myoblasts, adult stem cells, bone marrow cells, mesenchymal cells, embryonic stem cells, parenchymal cells, epithelial cells, endothelial cells, mesothelial cells, fibroblasts, osteoblasts, chondrocytes, exogenous cells, endogenous cells, stem cells, hematopoietic stem cells, bone-marrow derived progenitor cells, myocardial cells, skeletal cells, fetal cells, undifferentiated cells, multi-potent progenitor cells, unipotent progenitor cells, monocytes, cardiac myoblasts, skeletal myoblasts, macrophages, capillary endothelial cells, xenogenic cells, allogenic cells, and post-natal stem cells.

In some cases, the cell is an immune cell, a neuron, an epithelial cell, and endothelial cell, or a stem cell. In some cases, the immune cell is a T cell, a B cell, a monocyte, a natural killer cell, a dendritic cell, or a macrophage. In some cases, the immune cell is a cytotoxic T cell. In some cases, the immune cell is a helper T cell. In some cases, the immune cell is a regulatory T cell (Treg).

In some cases, the cell is a stem cell. Stem cells include adult stem cells. Adult stem cells are also referred to as somatic stem cells.

Adult stem cells are resident in differentiated tissue but retain the properties of self-renewal and ability to give rise to multiple cell types, usually cell types typical of the tissue in which the stem cells are found. Numerous examples of somatic stem cells are known to those of skill in the art, including muscle stem cells; hematopoietic stem cells; epithelial stem cells; neural stem cells; mesenchymal stem cells; mammary stem cells; intestinal stem cells; mesodermal stem cells; endothelial stem cells; olfactory stem cells; neural crest stem cells; and the like.

Stem cells of interest include mammalian stem cells, where the term “mammalian” refers to any animal classified as a mammal, including humans; non-human primates; domestic and farm animals; and zoo, laboratory, sports, or pet animals, such as dogs, horses, cats, cows, mice, rats, rabbits, etc. In some cases, the stem cell is a human stem cell. In some cases, the stem cell is a rodent (e.g., a mouse; a rat) stem cell. In some cases, the stem cell is a non-human primate stem cell.

In some embodiments, the stem cell is a hematopoietic stem cell (HSC). HSCs are mesoderm-derived cells that can be isolated from bone marrow, blood, cord blood, fetal liver, and yolk sac. HSCs are characterized as CD34⁺ and CD3⁻. HSCs can repopulate the erythroid, neutrophil-macrophage, megakaryocyte, and lymphoid hematopoietic cell lineages in vivo. In vitro, HSCs can be induced to undergo at least some self-renewing cell divisions and can be induced to differentiate to the same lineages as is seen in vivo. As such, HSCs can be induced to differentiate into one or more of erythroid cells, megakaryocytes, neutrophils, macrophages, and lymphoid cells.

In other embodiments, the stem cell is a neural stem cell (NSC). Neural stem cells (NSCs) are capable of differentiating into neurons, and glia (including oligodendrocytes, and astrocytes). A neural stem cell is a multipotent stem cell which is capable of multiple divisions, and under specific conditions can produce daughter cells which are neural stem cells, or neural progenitor cells that can be neuroblasts or glioblasts, e.g., cells committed to become one or more types of neurons and glial cells, respectively. Methods of obtaining NSCs are known in the art.

In other embodiments, the stem cell is a mesenchymal stem cell (MSC). MSCs originally derived from the embryonal mesoderm and isolated from adult bone marrow, can differentiate to form muscle, bone, cartilage, fat, marrow stroma, and tendon. Methods of isolating MSC are known in the art; and any known method can be used to obtain MSC. See, e.g., U.S. Pat. No. 5,736,396, which describes isolation of human MSC.

In some embodiments, the cell is a T cell. The invention is not limited by the type of T cell. The T cells may be selected from, for example, CD3+ T cells, CD8+ T cells, CD4+ T cells, natural killer (NK) T cells, alpha beta T cells, gamma delta T cells, or any combination thereof (e.g., a combination of CD4+ and CD8+ T cells).

In some embodiments, the T cells are naturally occurring T cells. For example, the T cells may be isolated from a subject sample. In some embodiments, the T cell is an anti-tumor T cell (e.g., a T cell with activity against a tumor (e.g., an autologous tumor) that becomes activated and expands in response to antigen). Anti-tumor T cells include, but are not limited to, T cells obtained from resected tumors or tumor biopsies (e.g., tumor infiltrating lymphocytes (TILs)) and a polyclonal or monoclonal tumor-reactive T cell (e.g., obtained by apheresis, expanded ex vivo against tumor antigens presented by autologous or artificial antigen-presenting cells). In some embodiments, the T cells are expanded ex vivo.

A cell is in some cases a plant cell. A plant cell can be a cell of a monocotyledon. A plant cell can be a cell of a dicotyledon. The cells can be root cells, leaf cells, cells of the xylem, cells of the phloem, cells of the cambium, apical meristem cells, parenchyma cells, collenchyma cells, sclerenchyma cells, and the like. Plant cells include cells of agricultural crops such as wheat, corn, rice, sorghum, millet, soybean, etc. Plant cells include cells of agricultural fruit and nut plants, e.g., plant that produce apricots, oranges, lemons, apples, plums, pears, almonds, etc.

A plant cell can be a cell of a major agricultural plant, e.g., Barley, Beans (Dry Edible), Canola, Corn, Cotton (Pima), Cotton (Upland), Flaxseed, Hay (Alfalfa), Hay (Non-Alfalfa), Oats, Peanuts, Rice, Sorghum, Soybeans, Sugarbeets, Sugarcane, Sunflowers (Oil), Sunflowers (Non-Oil), Sweet Potatoes, Tobacco (Burley), Tobacco (Flue-cured), Tomatoes, Wheat (Durum), Wheat (Spring), Wheat (Winter), and the like. As another example, the cell is a cell of a vegetable crops which include but are not limited to, e.g., alfalfa sprouts, aloe leaves, arrow root, arrowhead, artichokes, asparagus, bamboo shoots, banana flowers, bean sprouts, beans, beet tops, beets, bittermelon, bok choy, broccoli, broccoli rabe (rappini), brussels sprouts, cabbage, cabbage sprouts, cactus leaf (nopales), calabaza, cardoon, carrots, cauliflower, celery, chayote, chinese artichoke (crosnes), chinese cabbage, chinese celery, chinese chives, choy sum, chrysanthemum leaves (tung ho), collard greens, corn stalks, corn-sweet, cucumbers, daikon, dandelion greens, dasheen, dau mue (pea tips), donqua (winter melon), eggplant, endive, escarole, fiddle head ferns, field cress, frisee, gai choy (chinese mustard), gailon, galanga (siam, thai ginger), garlic, ginger root, gobo, greens, hanover salad greens, huauzontle, jerusalem artichokes, jicama, kale greens, kohlrabi, lamb's quarters (quilete), lettuce (bibb), lettuce (boston), lettuce (boston red), lettuce (green leaf), lettuce (iceberg), lettuce (lolla rossa), lettuce (oak leaf-green), lettuce (oak leaf-red), lettuce (processed), lettuce (red leaf), lettuce (romaine), lettuce (ruby romaine), lettuce (russian red mustard), linkok, lo bok, long beans, lotus root, mache, maguey (agave) leaves, malanga, mesculin mix, mizuna, moap (smooth luffa), moo, moqua (fuzzy squash), mushrooms, mustard, nagaimo, okra, ong choy, onions green, opo (long squash), ornamental corn, ornamental gourds, parsley, parsnips, peas, peppers (bell type), peppers, pumpkins, radicchio, radish sprouts, radishes, rape greens, rape greens, rhubarb, romaine (baby red), rutabagas, salicornia (sea bean), sinqua (angled/ridged luffa), spinach, squash, straw bales, sugarcane, sweet potatoes, swiss chard, tamarindo, taro, taro leaf, taro shoots, tatsoi, tepeguaje (guaje), tindora, tomatillos, tomatoes, tomatoes (cherry), tomatoes (grape type), tomatoes (plum type), tumeric, turnip tops greens, turnips, water chestnuts, yampi, yams (names), yu choy, yuca (cassava), and the like.

A cell is in some cases an arthropod cell. For example, the cell can be a cell of a sub-order, a family, a sub-family, a group, a sub-group, or a species of, e.g., Chelicerata, Myriapodia, Hexipodia, Arachnida, Insecta, Archaeognatha, Thysanura, Palaeoptera, Ephemeroptera, Odonata, anisoptera, Zygoptera, Neoptera, Exopterygota, Plecoptera, Embioptera, Orthoptera, Zoraptera, dermaptera, dictyoptera, Notoptera, Grylloblattidae, Mantophasmatidae, Phasmatodea, Blattaria, Isoptera, Mantodea, Parapneuroptera, Psocoptera, Thysanoptera, Phthiraptera, Hemiptera, Endopterygota or Holometabola, Hymenoptera, Coleoptera, Strepsiptera, Raphidioptera, Megaloptera, Neuroptera, Mecoptera, Siphonaptera, Diptera, Trichoptera, or Lepidoptera.

A cell is in some cases an insect cell. For example, in some cases, the cell is a cell of a mosquito, a grasshopper, a true bug, a fly, a flea, a bee, a wasp, an ant, a louse, a moth, or a beetle.

In some embodiments, introducing the system into a cell comprises administering the system to a subject. In some embodiments, the subject is human. The administering may comprise in vivo administration. In alternative embodiments, a vector is contacted with a cell in vitro or ex vivo and the treated cell, containing the system, is transplanted into a subject.

In some embodiments, the target nucleic acid is a nucleic acid endogenous to a target cell. In some embodiments, the target nucleic acid is a genomic DNA sequence. The term “genomic,” as used herein, refers to a nucleic acid sequence (e.g., a gene or locus) that is located on a chromosome in a cell.

In some embodiments, the target nucleic acid encodes a gene or gene product. The term “gene product,” as used herein, refers to any biochemical product resulting from expression of a gene. Gene products may be RNA or protein. RNA gene products include non-coding RNA, such as RNA, rRNA, micro RNA (miRNA), and small interfering RNA (siRNA), and coding RNA, such as messenger RNA (mRNA). In some embodiments, the target nucleic acid sequence encodes a protein or polypeptide.

The disclosed method may modify a target DNA sequence in a host cell so as to modulate expression of the target DNA sequence, e.g., expression of the target DNA sequence is increased, decreased, or completely eliminated (e.g., via deletion of a gene).

In another embodiment, the method of modifying a target sequence can be used to delete a nucleic acid sequence or portion thereof from a target sequence in a host cell by cleaving the target sequence and allowing the host cell to repair the cleaved sequence in the absence of an exogenously provided donor nucleic acid molecule. Deletion of a nucleic acid sequence in this manner can be used in a variety of applications, such as, for example, to remove disease-causing trinucleotide repeat sequences in neurons, to create gene knock-outs or knock-downs, and to generate mutations for disease models in research.

In some embodiments, the systems and methods described herein may be used to insert a gene or fragment thereof into a cell. In particular embodiments, the disclosed systems may be used to generate a cell that expresses a recombinant receptor. In some embodiments, the recombinant receptor is a T cell receptor (TCR) or a chimeric antigen receptor (CAR). Also provided herein are cells, e.g., a T cell, comprising a recombinant receptor and/or a nucleic acid encoding thereof and a system (e.g., nuclease and at least one gRNA) as described herein.

In some embodiments, the system and methods described herein may be used to genetically modify a plant or plant cell. As used herein, genetically modified plants include a plant into which has been introduced an exogenous polynucleotide. Genetically modified plants also include a plant that has been genetically manipulated such that endogenous nucleotides have been altered to include a mutation, such as a deletion, an insertion, a transition, a transversion, or a combination thereof. For instance, an endogenous coding region could be deleted. Such mutations may result in a polypeptide having a different amino acid sequence than was encoded by the endogenous polynucleotide. Another example of a genetically modified plant is one having an altered regulatory sequence, such as a promoter, to result in increased or decreased expression of an operably linked endogenous coding region. The genetically modified plant may promote a desired phenotypic or genotypic plant trait.

Genetically modified plants can potentially have improved crop yields, enhanced nutritional value, and increased shelf life. They can also be resistant to unfavorable environmental conditions, insects, and pesticides. The present systems and methods have broad applications in gene discovery and validation, mutational and cisgenic breeding, and hybrid breeding. The present systems and methods may facilitate the production of a new generation of genetically modified crops with various improved agronomic traits such as herbicide resistance, herbicide tolerance, drought tolerance, male sterility, insect resistance, abiotic stress tolerance, modified fatty acid metabolism, modified carbohydrate metabolism, modified seed yield, modified oil percent, modified protein percent, resistance to bacterial disease, disease (e.g. bacterial, fungal, and viral) resistance, high yield, and superior quality. The present systems and methods may also facilitate the production of a new generation of genetically modified crops with optimized fragrance, nutritional value, shelf-life, pigmentations (e.g., lycopene content), starch content (e.g., low-gluten wheat), toxin levels, propagation and/or breeding and growth time. See, for example, CRISPR/Cas Genome Editing and Precision Plant Breeding in Agriculture (Chen et al., Annu Rev Plant Biol. 2019 Apr. 29; 70:667-69), incorporated herein by reference.

The present system and method may confer one or more of the following traits to the plant cell: herbicide tolerance, drought tolerance, male sterility, insect resistance, abiotic stress tolerance, modified fatty acid metabolism, modified carbohydrate metabolism, modified seed yield, modified oil percent, modified protein percent, resistance to bacterial disease, resistance to fungal disease, and resistance to viral disease.

The present disclosure provides for a modified plant cell produced by the present system and method, a plant comprising the plant cell, and a seed, fruit, plant part, or propagation material of the plant. Transformed or genetically modified plant cells of the present disclosure may be as populations of cells, or as a tissue, seed, whole plant, stem, fruit, leaf, root, flower, stem, tuber, grain, animal feed, a field of plants, and the like. The present disclosure provides a transgenic plant. The transgenic plant may be homozygous or heterozygous for the genetic modification. Also provided by the present disclosure are transformed or genetically modified plant cells, tissues, plants, and products that contain the transformed or genetically modified plant cells. The present disclosure further encompasses the progeny, clones, cell lines or cells of the transgenic plants.

The present system and method may be used to modify a plant stem cell. The present disclosure further provides progeny of a genetically modified cell, where the progeny can comprise the same genetic modification as the genetically modified cell from which it was derived. The present disclosure further provides a composition comprising a genetically modified cell.

In one embodiment, the transformed or genetically modified cells, and tissues and products comprise a nucleic acid integrated into the genome, and production by plant cells of a gene product due to the transformation or genetic modification.

Methods of introducing exogenous nucleic acids into plant cells are well known in the art. Such plant cells are considered “transformed.” DNA constructs can be introduced into plant cells by various methods, including, but not limited to PEG- or electroporation-mediated protoplast transformation, tissue culture or plant tissue transformation by biolistic bombardment, or the Agrobacterium-mediated transient and stable transformation. The transformation can be transient or stable transformation. Suitable methods also include viral infection (such as double stranded DNA viruses), transfection, conjugation, protoplast fusion, electroporation, particle gun technology, calcium phosphate precipitation, direct microinjection, silicon carbide whiskers technology, Agrobacterium-mediated transformation, and the like. The choice of method is generally dependent on the type of cell being transformed and the circumstances under which the transformation is taking place (i.e., in vitro, ex vivo, or in vivo). Transformation methods based upon the soil bacterium Agrobacterium tumefaciens are useful for introducing an exogenous nucleic acid molecule into a vascular plant. The wild-type form of Agrobacterium contains a Ti (tumor-inducing) plasmid that directs production of tumorigenic crown gall growth on host plants. Transfer of the tumor-inducing T-DNA region of the Ti plasmid to a plant genome requires the Ti plasmid-encoded virulence genes as well as T-DNA borders, which are a set of direct DNA repeats that delineate the region to be transferred. An Agrobacterium-based vector is a modified form of a Ti plasmid, in which the tumor inducing functions are replaced by the nucleic acid sequence of interest to be introduced into the plant host.

Agrobacterium-mediated transformation generally employs cointegrate vectors or binary vector systems, in which the components of the Ti plasmid are divided between a helper vector, which resides permanently in the Agrobacterium host and carries the virulence genes, and a shuttle vector, which contains the gene of interest bounded by T-DNA sequences. A variety of binary vectors are well known in the art and are commercially available, for example, from Clontech (Palo Alto, Calif.). Methods of coculturing Agrobacterium with cultured plant cells or wounded tissue such as leaf tissue, root explants, hypocotyledons, stem pieces or tubers, for example, also are well known in the art. See., e.g., Glick and Thompson, (eds.), Methods in Plant Molecular Biology and Biotechnology, Boca Raton, Fla.: CRC Press (1993), incorporated herein by reference.

Microprojectile-mediated transformation also can be used to produce a transgenic plant. This method, first described by Klein et al. (Nature 327:70-73 (1987), incorporated herein by reference), relies on microprojectiles such as gold or tungsten that are coated with the desired nucleic acid molecule by precipitation with calcium chloride, spermidine, or polyethylene glycol. The microprojectile particles are accelerated at high speed into an angiosperm tissue using a device such as the BIOLISTIC PD-1000 (Biorad; Hercules Calif.).

In one embodiment, the present systems and methods may be adapted to use in plants. In one embodiment, a series of plant-specific RNA-guided Genome Editing vectors (pRGE plasmids) are provided for expression of the present system in plants. The vectors may be optimized for transient expression of the present system in plant protoplasts, or for stable integration and expression in intact plants via the Agrobacterium-mediated transformation. In one aspect, the vector constructs include a nucleotide sequence comprising a DNA-dependent RNA polymerase III promoter, wherein the promoter is operably linked to a gRNA molecule and a Pol III terminator sequence, and a nucleotide sequence comprising a DNA-dependent RNA polymerase II promoter operably linked to a nucleic acid sequence encoding the nuclease.

In certain embodiments, the present systems and methods use a monocot promoter to drive the expression of one or more components of the present systems (e.g., gRNA) in a monocot plant. In certain embodiments, the present systems and methods use a dicot promoter to drive the expression of one or more components of the present systems (e.g., gRNA) in a dicot plant. In some embodiments, the present system is transiently expressed in plant protoplasts. Vectors for transient transformation of plants include, but are not limited to, pRGE3, pRGE6, pRGE31, and pRGE32. In some embodiment, the vector may be optimized for use in a particular plant type or species, such as pStGE3.

In one embodiment, the present system may be stably integrated into the plant genome, for example via Agrobacterium-mediated transformation. Thereafter, one or more components of the present system (e.g., the transgene) may be removed by genetic cross and segregation, which may lead to the production of non-transgenic, but genetically modified plants or crops. In one embodiment, the vector is optimized for Agrobacterium-mediated transformation. In one embodiment, the vector for stable integration is pRGEB3, pRGEB6, pRGEB31, pRGEB32, or pStGEB3.

The present system may be used in various bacterial hosts, including human pathogens that are medically important, and bacterial pests that are key targets within the agricultural industry, as well as antibiotic resistant versions thereof.

The system and method may be designed to target any gene or any set of genes, such as virulence or metabolic genes, for clinical and industrial applications in other embodiments. For example, the present systems and methods may be used to target and eliminate virulence genes from the population, to perform in situ gene knockouts, or to stably introduce new genetic elements to the metagenomic pool of a microbiome. The present systems and methods may be used to treat a multi-drug resistance bacterial infection in a subject. The present systems and methods may be used for genomic engineering within complex bacterial consortia.

The present systems and methods may be used to inactivate microbial genes. In some embodiments, the gene is an antibiotic resistance gene. For example, the coding sequence of bacterial resistance genes may be disrupted in vivo by insertion of a DNA sequence, leading to non-selective re-sensitization to drug treatment.

The components of the composition or system may be administered with a pharmaceutically acceptable carrier or excipient as a pharmaceutical composition. In some embodiments, the components of the present system may be mixed, individually or in any combination, with a pharmaceutically acceptable carrier to form pharmaceutical compositions, which are also within the scope of the present disclosure.

In some embodiments, an effective amount of the components of the present system or compositions as described herein can be administered. Within the context of the present disclosure, the term “effective amount” refers to that quantity of the components of the system such that modification of the target nucleic acid is achieved.

The methods described here also provide for treating a disease or condition in a subject. In some embodiments, the systems and methods are used to treat a pathogen or parasite on or in a subject by altering the pathogen or parasite. In some embodiments, the systems and methods target a “disease-associated” gene. The term “disease-associated gene,” refers to any gene or polynucleotide whose gene products are expressed at an abnormal level or in an abnormal form in cells obtained from a disease-affected individual as compared with tissues or cells obtained from an individual not affected by the disease. A disease-associated gene may be expressed at an abnormally high level or at an abnormally low level, where the altered expression correlates with the occurrence and/or progression of the disease. A disease-associated gene also refers to a gene, the mutation or genetic variation of which is directly responsible or is in linkage disequilibrium with a gene(s) that is responsible for the etiology of a disease. Examples of genes responsible for such “single gene” or “monogenic” diseases include, but are not limited to, adenosine deaminase, α-1 antitrypsin, cystic fibrosis transmembrane conductance regulator (CFTR), β-hemoglobin (HBB), oculocutaneous albinism II (OCA2), Huntingtin (HTT), dystrophia myotonica-protein kinase (DMPK), low-density lipoprotein receptor (LDLR), apolipoprotein B (APOB), neurofibromin 1 (NF1), polycystic kidney disease 1 (PKD1), polycystic kidney disease 2 (PKD2), coagulation factor VIII (F8), dystrophin (DMD), phosphate-regulating endopeptidase homologue, X-linked (PHEX), methyl-CpG-binding protein 2 (MECP2), and ubiquitin-specific peptidase 9Y, Y-linked (USP9Y). Other single gene or monogenic diseases are known in the art and described in, e.g., Chial, H. Rare Genetic Disorders: Learning About Genetic Disease Through Gene Mapping, SNPs, and Microarray Data, Nature Education 1(1):192 (2008); Online Mendelian Inheritance in Man (OMIM); and the Human Gene Mutation Database (HGMD). In another embodiment, the target genomic DNA sequence can comprise a gene, the mutation of which contributes to a particular disease in combination with mutations in other genes. Diseases caused by the contribution of multiple genes which lack simple (i.e., Mendelian) inheritance patterns are referred to in the art as a “multifactorial” or “polygenic” disease. Examples of multifactorial or polygenic diseases include, but are not limited to, asthma, diabetes, epilepsy, hypertension, bipolar disorder, and schizophrenia. Certain developmental abnormalities also can be inherited in a multifactorial or polygenic pattern and include, for example, cleft lip/palate, congenital heart defects, and neural tube defects. In another embodiment, the target DNA sequence can comprise a cancer oncogene.

The present disclosure provides for gene editing methods that can ablate a disease-associated gene (e.g., a cancer oncogene), which in turn can be used for in vivo gene therapy for patients. In some embodiments, the gene editing methods include donor nucleic acids comprising therapeutic genes.

When utilized as a method of treatment, the effective amount may depend on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. In some embodiments, the effective amount alleviates, relieves, ameliorates, improves, reduces the symptoms, or delays the progression of any disease or disorder in the subject. In some embodiments, the subject is a human.

A wide range of additional therapies may be used in conjunction with the methods of the present disclosure. The additional therapy may be administration of an additional therapeutic agent or may be an additional therapy not connected to administration of another agent. Such additional therapies include, but are not limited to, surgery, immunotherapy, radiotherapy. The additional therapy may be administered at the same time as the above methods. In some embodiments, the additional therapy may precede or follow the treatment of the disclosed methods by time intervals ranging from hours to months.

In some embodiments, a therapeutically effective amount of a system (e.g., nuclease and/or gRNA) or compositions described herein, is administered alone or in combination with a therapeutically effective amount of at least one additional therapeutic agent. In some embodiments, effective combination therapy is achieved with a single composition or pharmacological formulation or with two distinct compositions or formulations, administered at the same time or separated by a time interval. The at least one additional therapeutic agent may comprise any manner of therapeutic, including protein, small molecule, nucleic acids, and the like. For example, exemplary additional therapeutic agents include, but are not limited to, immune modulators, chemotherapeutic agents, a nucleic acid (e.g., mRNA, aptamers, antisense oligonucleotides, ribozyme nucleic acids, interfering RNAs, antigene nucleic acids), decongestants, steroids, analgesics, antimicrobial agents, immunotherapies, or any combination thereof.

In the context of the present disclosure insofar as it relates to any of the disease conditions recited herein, the terms “treat,” “treatment,” and the like mean to relieve or alleviate at least one symptom associated with such condition, or to slow or reverse the progression of such condition. Within the meaning of the present disclosure, the term “treat” also denotes to arrest, delay the onset (e.g., the period prior to clinical manifestation of a disease) and/or reduce the risk of developing or worsening a disease. For example, in connection with cancer the term “treat” may mean elimination or reduction of a patient's tumor burden, or a prevention, delay, or inhibition of metastasis, etc.

The phrase “pharmaceutically acceptable,” as used in connection with compositions and/or cells of the present disclosure, refers to molecular entities and other ingredients of such compositions that are physiologically tolerable and do not typically produce untoward reactions when administered to a subject (e.g., a mammal, a human). Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans. “Acceptable” means that the carrier is compatible with the active ingredient of the composition (e.g., the nucleic acids, vectors, cells, or therapeutic antibodies) and does not negatively affect the subject to which the composition(s) are administered. Any of the pharmaceutical compositions and/or cells to be used in the present methods can comprise pharmaceutically acceptable carriers, excipients, or stabilizers in the form of lyophilized formations or aqueous solutions.

Pharmaceutically acceptable carriers, including buffers, are well known in the art, and may comprise phosphate, citrate, and other organic acids; antioxidants including ascorbic acid and methionine; preservatives; low molecular weight polypeptides; proteins, such as serum albumin, gelatin, or immunoglobulins; amino acids; hydrophobic polymers; monosaccharides; disaccharides; and other carbohydrates; metal complexes; and/or non-ionic surfactants. See, e.g., Remington: The Science and Practice of Pharmacy 20th Ed. (2000) Lippincott Williams and Wilkins, Ed. K. E. Hoover.

In some cases, desirable delivery systems provide for roughly uniform distribution and have controllable rates of release of their components (e.g., vectors, proteins, nucleic acids) in vivo. A variety of different media are described below that are useful in creating composition delivery systems. It is not intended that any one medium is limiting to the present invention. Note that any medium may be combined with another medium or carrier; for example, in one embodiment a polymer microparticle attached to a compound may be combined with a gel medium. An implantable device can be used to deliver a nuclease, or a nucleic acid encoding thereof, and gRNA, or a nucleic acid encoding thereof, to, for example, a target cell in vivo.

Carriers or mediums contemplated include materials such as gelatin, collagen, cellulose esters, dextran sulfate, pentosan polysulfate, chitin, saccharides, albumin, fibrin sealants, synthetic polyvinyl pyrrolidone, polyethylene oxide, polypropylene oxide, block polymers of polyethylene oxide and polypropylene oxide, polyethylene glycol, acrylates, acrylamides, methacrylates including, but not limited to, 2-hydroxyethyl methacrylate, poly(ortho esters), cyanoacrylates, gelatin-resorcin-aldehyde type bioadhesives, polyacrylic acid and copolymers and block copolymers thereof.

In some cases, a carrier/medium can include a microparticle. Microparticles can include, but are not limited to, liposomes, nanoparticles, microspheres, nanospheres, microcapsules, and nanocapsules. In some cases, microparticle can include one or more of the following: a poly(lactide-co-glycolide), aliphatic polyesters including, but not limited to, poly-glycolic acid and poly-lactic acid, hyaluronic acid, modified polysaccharides, chitosan, cellulose, dextran, polyurethanes, polyacrylic acids, pseudo-poly(amino acids), polyhydroxybutyrate-related copolymers, polyanhydrides, polymethylmethacrylate, poly(ethylene oxide), lecithin and phospholipids—in any combination thereof.

In some cases, a carrier/medium can include a liposome that is capable of attaching and releasing therapeutic agents (e.g., the subject nucleic acids and/or proteins). Liposomes are microscopic spherical lipid bilayers surrounding an aqueous core that are made from amphiphilic molecules such as phospholipids. For example, a liposome may trap a therapeutic agent between the hydrophobic tails of the phospholipid micelle. Water soluble agents can be entrapped in the core and lipid-soluble agents can be dissolved in the shell-like bilayer. Liposomes have a special characteristic in that they enable water soluble and water insoluble chemicals to be used together in a medium without the use of surfactants or other emulsifiers. Liposomes can form spontaneously by forcefully mixing phospholipids in aqueous media. Water soluble compounds are dissolved in an aqueous solution capable of hydrating phospholipids. Upon formation of the liposomes, therefore, these compounds are trapped within the aqueous liposomal center. The liposome wall, being a phospholipid membrane, holds fat soluble materials such as oils. Liposomes provide controlled release of incorporated compounds. In addition, liposomes can be coated with water soluble polymers, such as polyethylene glycol to increase the pharmacokinetic half-life.

In some embodiments, a cationic or anionic liposome is used as part of a subject composition or method, or liposomes having neutral lipids can also be used. Cationic liposomes can include negatively-charged materials by mixing the materials and fatty acid liposomal components and allowing them to charge-associate. The choice of a cationic or anionic liposome depends upon the desired pH of the final liposome mixture.

Any element of any suitable CRISPR/Cas gene editing system known in the art can be employed in the systems and methods described herein, as appropriate. CRISPR/Cas gene editing technology is described in detail in, for example, U.S. Pat. Nos. 8,546,553, 8,697,359; 8,771,945; 8,795,965; 8,865,406; 8,871,445; 8,889,356; 8,889,418; 8,895,308; 8,9066,616; 8,932,814; 8,945,839; 8,993,233; 8,999,641; 9,115,348; 9,149,049; 9,493,844; 9,567,603; 9,637,739; 9,663,782; 9,404,098; 9,885,026; 9,951,342; 10,087,431; 10,227,610; 10,266,850; 10,601,748; 10,604,771; and 10,760,064; and U.S. Patent Application Publication Nos. US2010/0076057; US2014/0113376; US2015/0050699; US2015/0031134; US2014/0357530; US2014/0349400; US2014/0315985; US2014/0310830; US2014/0310828; US2014/0309487; US2014/0294773; US2014/0287938; US2014/0273230; US2014/0242699; US2014/0242664; US2014/0212869; US2014/0201857; US2014/0199767; US2014/0189896; US2014/0186919; US2014/0186843; and US2014/0179770, each incorporated herein by reference.

### Kits

Also within the scope of the present disclosure are kits that include the compositions, systems, or components thereof as disclosed herein.

For example the kits may contain one or more reagents or other components useful, necessary, or sufficient for practicing any of the methods described herein, such as, editing reagents (nuclease, guide RNAs, vectors, compositions, etc.), transfection or administration reagents, negative and positive control samples (e.g., cells, template DNA), cells, containers housing one or more components (e.g., microcentrifuge tubes, boxes), detectable labels, detection and analysis instruments, software, instructions, and the like.

The kit may include instructions for use in any of the methods described herein. The instructions can comprise a description of administration of the present system or composition to a subject to achieve the intended effect. The instructions generally include information as to dosage, dosing schedule, and route of administration for the intended treatment. The kit may further comprise a description of selecting a subject suitable for treatment based on identifying whether the subject is in need of the treatment.

The kits provided herein are in suitable packaging. Suitable packaging includes, but is not limited to, vials, bottles, jars, flexible packaging, and the like. A kit may have a sterile access port (for example, the container may be an intravenous solution bag or a vial having a stopper pierceable by a hypodermic injection needle). The container may also have a sterile access port.

The packaging may be unit doses, bulk packages (e.g., multi-dose packages) or sub-unit doses. Instructions supplied in the kits of the disclosure are typically written instructions on a label or package insert. The label or package insert indicates that the pharmaceutical compositions are used for treating, delaying the onset, and/or alleviating a disease or disorder in a subject.

Kits optionally may provide additional components such as buffers and interpretive information. Normally, the kit comprises a container and a label or package insert(s) on or associated with the container. In some embodiment, the disclosure provides articles of manufacture comprising contents of the kits described above.

The kit may further comprise a device for holding or administering the present system or composition. The device may include an infusion device, an intravenous solution bag, a hypodermic needle, a vial, and/or a syringe.

### EXAMPLES

The following are examples of the present invention and are not to be construed as limiting.

### Example 1

### Nuclease and Guide RNA Vectors

Identification of Single guide RNA vector sets Nuclease sequences (SEQ ID NOs: 1-250) were identified as candidate CRISPR Type V nucleases with Cas12f-like features. Single guide RNA (sgRNA) vectors were designed for nucleases SEQ ID NOs: 1-54 based on their predicted crRNA and tracrRNA binding and folding patterns (Table 5). The designed sgRNAs were placed downstream of the U6 promoter with a starting G, and then placed upstream of the spacer sequence (Table 6).

Nuclease expression vectors Codon-optimized genes encoding candidate nucleases (nuclease amino acid sequences SEQ ID NOs: 20-29 and 36) were synthesized and cloned into the mammalian expression vector under the CMV promoter, pTwist_CMV (Twist Biosciences). The cloned nucleases were placed into the expression vector with a SV40 Nuclear Localization Sequence (NLS) fused to the N-terminal and a nucleoplasmin NLS on their C-terminal, followed by a 3×HA tag. A similar vector was created with Un1Cas12f1 (SEQ ID NO: 471).

### Example 2

### Editing Activity in Human Cells

Nucleases SEQ ID NOs: 21, 24 and 36 were tested in HEK293T cells through plasmid transfection using Mirus Transit X2 reagent. 50,000 cells were plated per well of a 96 well plate and immediately transfected with 100 ng of nuclease expression vector and 100 ng of the corresponding sgRNA vector shown in Table 1.

TABLE 1

| | | Corresponding |
|---|---|---|
| sgRNA | sgRNA sequence | Nuclease |
| SEQ ID | TTGAAATAAAATGAATTTCAAACCCCTTCGGGGGTGGGCGTGTTGGAGCGC | SEQ ID NO: 21 |
| NO: 310 | CTTAATTTGAGGTGCAGAATCCAAAAACTGCGACGATGTAGGTCGTTTCAG | |
| | TCTCTGCGCACTCAAAAAATTCACTTGATTaTTCAAGTGAATATCCAAC | |
| SEQ ID | ATTAAACCCCATTATGGGGTGGGCGTGTTGGAACGCCTTAGTTTGAGGTTT | SEQ ID NO: 24 |
| NO: 313 | GAAAAACAAATTTGGGTTATATTTGGTAATCTTAATGTTCAAGCACTCAAA | |
| | AAATTCACTTAAATTAaTTTAAGTGGATATCCAAC | |
| SEQ ID | CTTTCGGGATGGGCGCGTTGGAGCGCCTTGGTTTGAGGTGAGGACACCATA | SEQ ID NO: 36 |
| NO: 325 | ATCCGCATAATGAATATTGTACGGATGTCCCTGCACTCGAAAAGTTCACTT | |
| | GATTaTCAAGTGAATATCCAAC | |

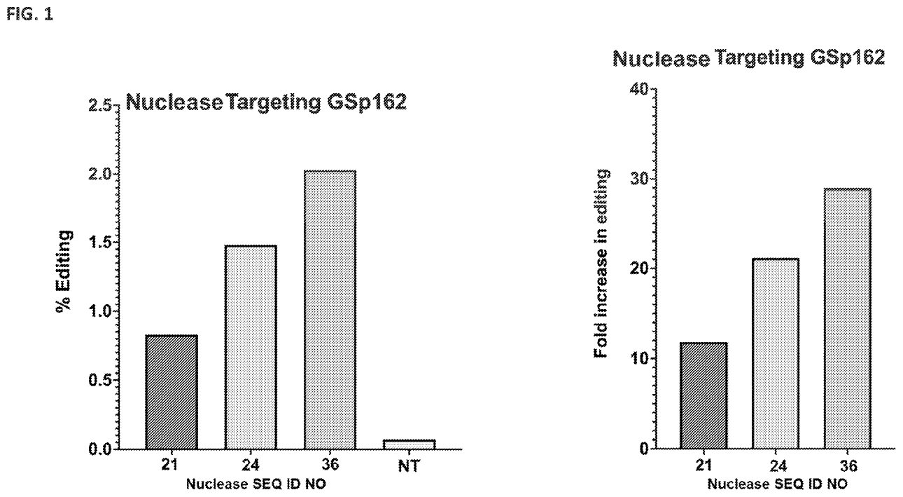

Samples were incubated for 72 h and harvested with QuickExtract (Lucigen). About 200 ng of genomic DNA was amplified using KAPA HiFi polymerase and primers specific to the targeted region on chromosome 3 with Illumina adapters ACACTCTTTCCCTACACGACGCTCTTCCGATCTgtaatgagcaaccttgagggatcagg (SEQ ID NO: 506) and GACTGGAGTTCAGACGTGTGCTCTTCCGATCTctcatggcaaaagcagtaatcagaac (SEQ ID NO: 507). 2 uL of this first 25 uL PCR was input to a second PCR using Illumina P7 barcoded primers from New England BioLabs kit #E6609S. PCR products were checked on a 2% agarose gel for purity and cleaned via ZYMO kit #D4034. Samples were then sequenced on the Illumina MiSeq system, which returned 100,000-400,000 150 bp paired-end reads per sample. Editing analysis was performed by CRISPResso2 with the option “--cleavage_offset 1” (Clement, Kendell, et al. “CRISPResso2 provides accurate and rapid genome editing sequence analysis.” Nature biotechnology 37.3 (2019): 224-226.). The percentage of nucleotide insertion or deletion mutations (indels) around the cut site was calculated for transfected and non-transfected (NT) cells without including substitution-only mutations. The indel percentages of transfected cells were divided by the indel percentage of non-transfected cells to calculate fold change in editing. Results are shown in FIG. 1.

### Example 3

### Engineered Single Guide RNAs

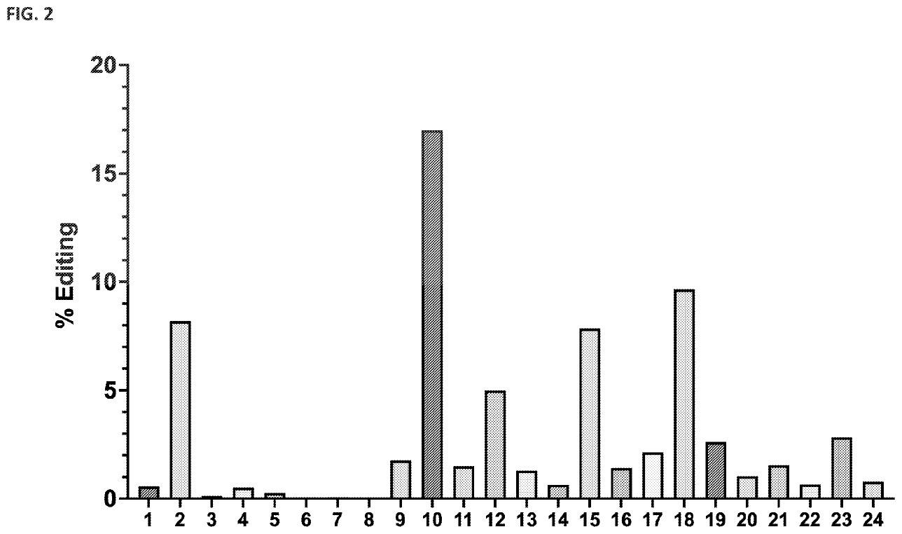

Engineered single guide RNA (sgRNA) vectors for nucleases SEQ ID NOs: 21, 24 and 36 were designed with varying lengths as shown in Table 2. The designed sgRNAs were placed downstream of the U6 promoter with a starting G, and then placed upstream of the spacer sequence, CACACACACAGTGGGCTACC (SEQ ID NO: 423), which targets an intergenic region of chromosome 3 of the human genome and has a 5′ TTTG PAM sequence. Nucleases SEQ ID NOs: 21, 24 and 36 were tested in HEK293T cells through plasmid transfection using Mirus Transit X2 reagent. 50,000 cells were plated per well of a 96 well plate and immediately transfected with 100 ng of nuclease expression vector and 100 ng of the corresponding sgRNA vector. Samples were incubated for 72 h and harvested with QuickExtract (Lucigen). Genomic DNA was amplified around the targeted region on chromosome 3 and sequenced by Sanger sequencing. TIDE (Tracking of Indels by Decomposition) analysis was performed following the method of Brinkman et al., (Brinkman E K, Chen T, Amendola M, van Steensel B. Nucleic Acids Res. 2014; 42(22):e168, incorporated herein by reference in its entirety) and recommendations at tide.nki.nl. Results are shown in FIG. 2. Table 3 shows the corresponding nuclease and guide RNA sequences for each numerical sample. Editing was improved using certain truncations of the sgRNAs.

### Example 4

### Editing Activity in Human Cells

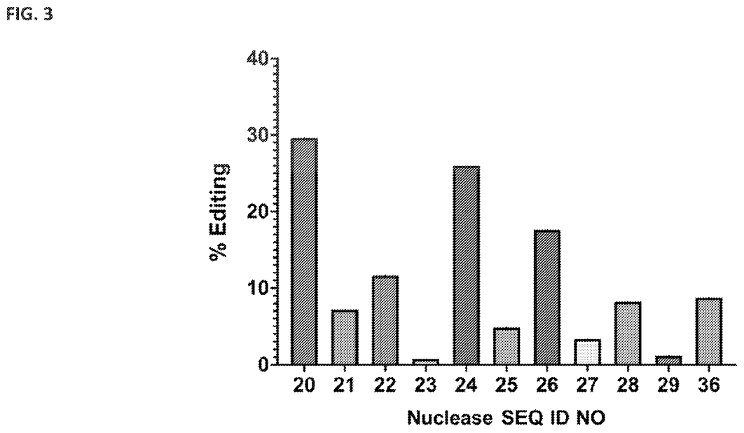

The editing activity of nucleases SEQ ID NOs: 20-29 and 36 were tested in HEK293T cells targeting Kim-T1 (SEQ ID NO: 423) with sgRNA of SEQ ID NO: 346 following the methods described in Example 2. Results shown in FIG. 3 indicated that the selected nucleases had editing activity in human cells.

### Example 5

### Off-Target Editing Activity

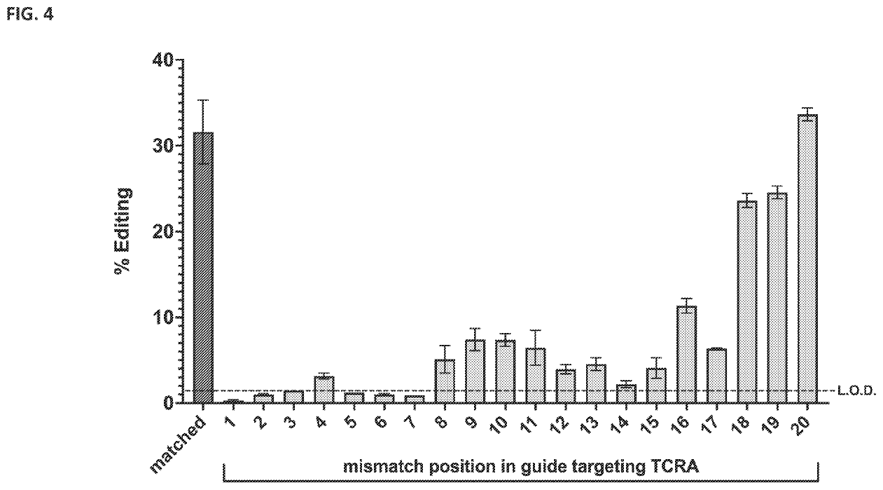

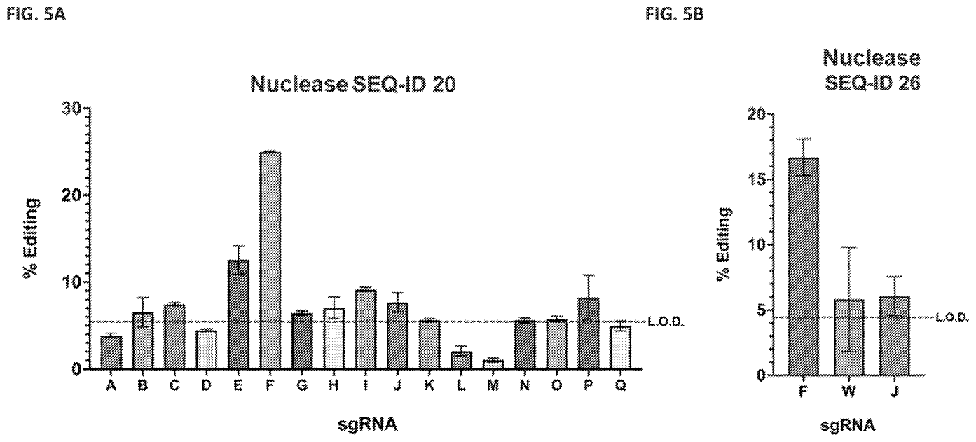

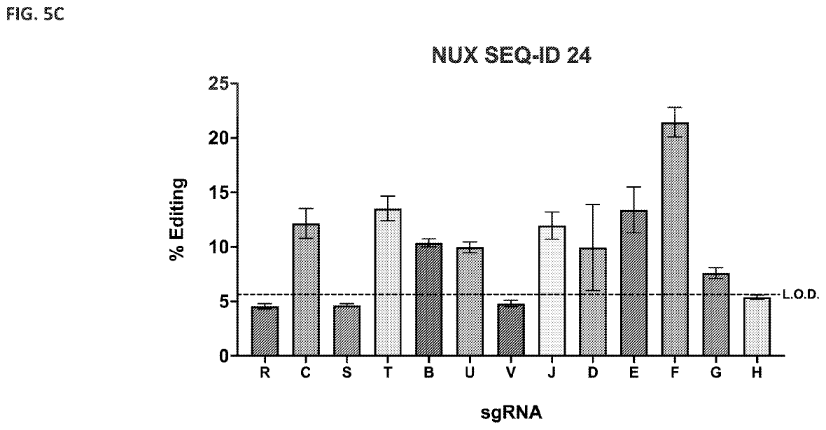

The nuclease SEQ ID NO: 20 was tested as described in Example 3 with either a guide matching the TCRA gene (SEQ-ID NO: 430) or a guide with a single mismatch for TCRA at different positions (SEQ-ID Nos: 433-452) The mismatched guides acted as artificial off-targets to determine the propensity of the nuclease to edit with mismatches at each position of the guide. Editing efficiency was measured for the matched guide and mismatched guides with Sanger sequencing as described in Example 3. The resulting amplicons were Sanger sequenced and TIDE analysis was performed following the method of Brinkman et al., 2014 as well as TIDE's website (tide.nki.nl) recommendations. Non-transfected cells were also harvested, amplified, and sequenced via the same methods to set a limit of detection (L.O.D.), under which editing levels cannot be determined. Results for the editing efficiency with the single mismatch guide RNAs are shown in FIG. 4.

### Example 6

### Guide RNA Modifications

Single guide RNA (sgRNA) constructs for targeting Kim-T1 were designed based on their predicted crRNA and tracrRNA binding and folding patterns and cloned into vectors as described in Example 1. The sgRNAs (Table 8) were tested with nucleases having SEQ ID NOs: 20, 24 and 26 following the methods as described in Example 3. Results are shown in FIGS. 5A-5C for each of SEQ ID NOs: 20, 24 and 26, respectively and in FIG. 5D for additional sequences with SEQ ID NO: 20. A putative structure of the sgRNA and the modifications are shown in FIG. 5E. Surprisingly, some of the modifications such as those in SEQ ID NO: 346, which removed a predicted stem-loop, allowed the sgRNA construct to function well with multiple nucleases. Additionally surprising, a number of truncations located within the stem and upper loop retained functionality when paired with nuclease SEQ ID NO:20.

### Example 7

### Guide RNA Modifications

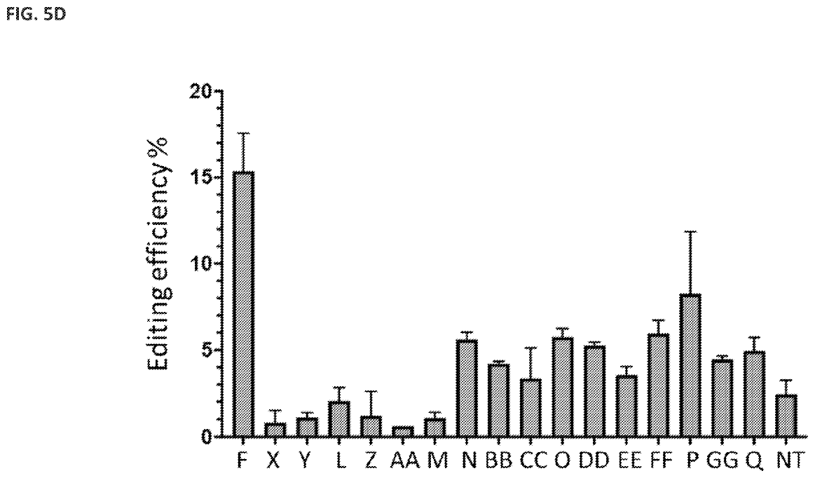

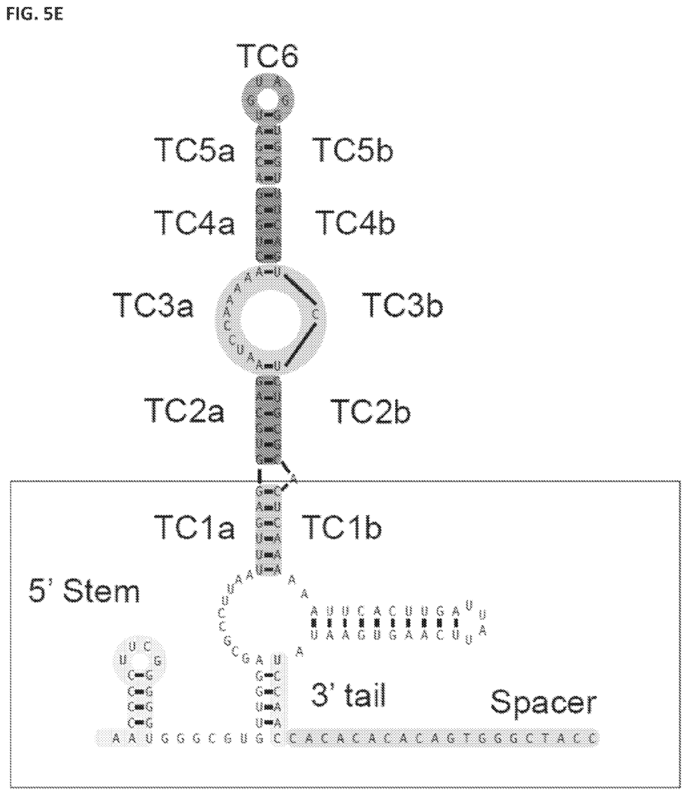

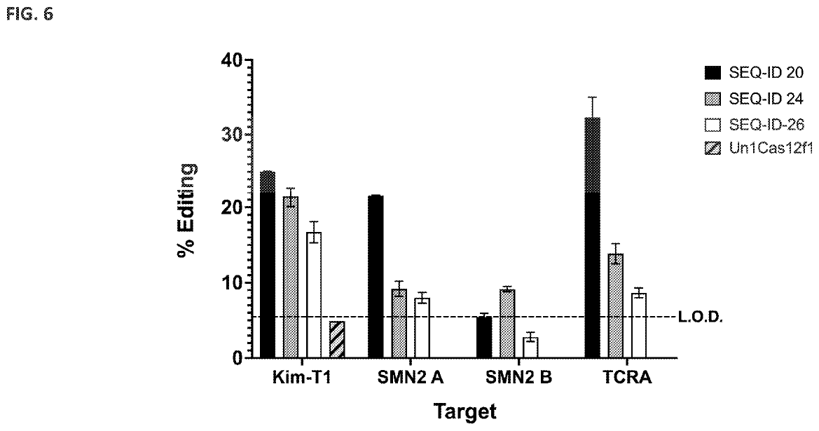

Editing activity for nucleases having SEQ ID NOs: 20, 24, 26 and Un1Cas12f1 (SEQ ID NO: 471) was compared over different target sites using the sgRNA having SEQ ID NO: 346 following the methods as described in Example 3. Results are shown in FIG. 6. The results indicated that each of the nucleases was able to edit at a variety of genomic target sites to varying levels. Surprisingly, Un1Cas12f1 when paired with the sgRNA having SEQ ID NO: 346 did not show editing above background levels at the Kim-T1 site, whereas the other 3 nucleases showed editing activity with this sgRNA.

### Example 8

### TracrRNA Modifications

The editing activities of nucleases SEQ ID NOs: 20 and 21 were compared with sgRNAs having small deletions in the tracrRNA sequence following the methods as described in Example 3. The tracrRNA deletions and editing results are shown in Table 9.

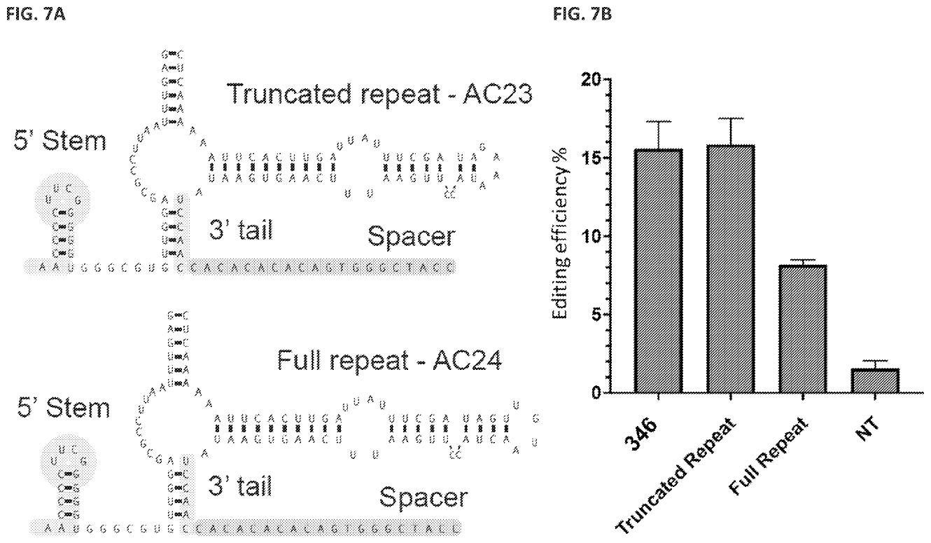

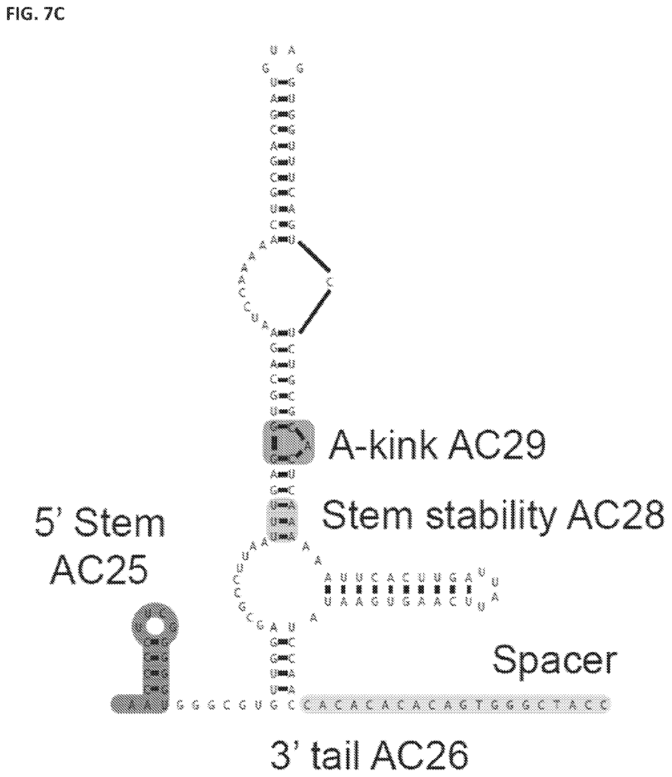

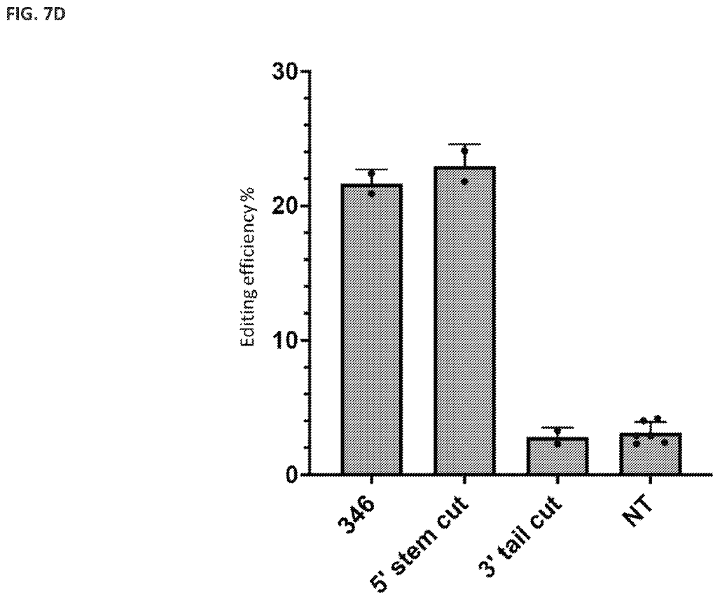

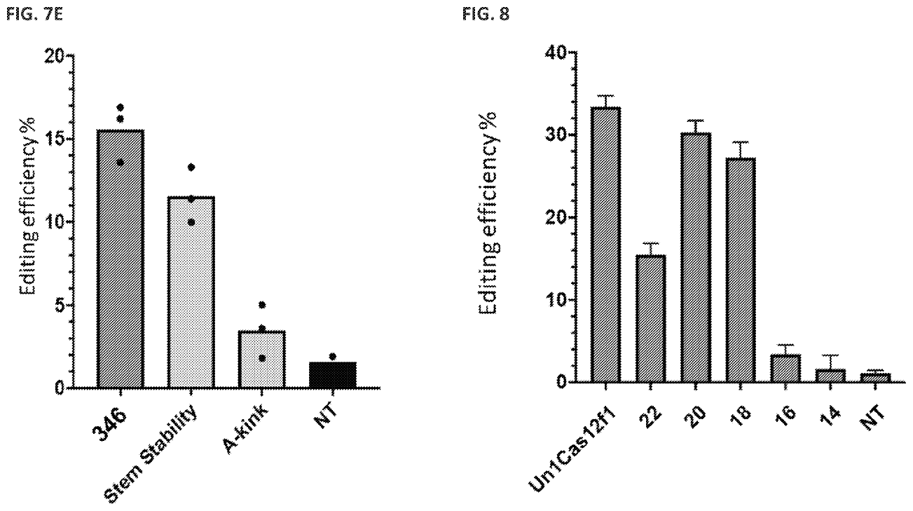

Nuclease SEQ ID NO:20 was then tested on a number of sgRNA modifications that altered the predicted structure of the tracrRNA sequence. Two configurations were tested having a longer repeat or a truncated repeat (see FIG. 7A) and compared to a modification having a truncated 5′ stem (SEQ ID NO: 346). Notably, having the full repeat was detrimental to the editing activity when compared to other truncated versions (FIG. 7B).

To further investigate the relationship of the tracrRNA sequence for these nucleases, further modifications were created. Starting with SEQ ID NO: 346, a portion of 5′ stem as well as 3′ tail of the tracrRNA were removed to evaluate their importance in the editing efficiency (FIG. 7C). Removing 5′ stem further did not impact editing, whereas removing 3′ tail of the tracrRNA was very detrimental to editing and had an efficiency similar to the values observed for non-targeted cells (FIG. 7D).

To further assess the role of the base of the stem, this sequence was modified to strengthen the base-pairing by changing A-T into G-C shown “Stem stability” and separately by removing the kink inserted by an unpaired A single nucleotide right above (FIG. 7C). Improving stability of the stem changed the predicted AG of the structure, however it did not improve the editing efficiency of nuclease SEQ ID NO: 20. Removing the A-kink completely abrogated editing capabilities of the nuclease (FIG. 7E).

### Example 9

### Spacer Modifications

The editing activities of nuclease SEQ ID NO: 20 was assessed for editing activity on sgRNA having variations in the length of the spacer sequence, following the methods as described in Example 3. Editing results are shown in FIG. 8. A spacer length of 18-20 nucleotides was optimal for editing activity.

### Example 10

### PAM Preferences

PAM sequences were tested for their effect on nucleases' editing efficiency following the method using spacer 3 of Walton et al. (Walton R T, et al., Science. 2020 Apr. 17; 368(6488):290-296, incorporated herein by reference in its entirety). Briefly, a spacer capable of targeting a randomized PAM plasmid library made with 10-bp of randomized PAMs incorporated downstream of the TracrRNA and repeat regions of the gRNA. The effective PAMs for the nucleases were depleted during the process, and the remaining PAMs were revealed by next-generation sequencing (NGS). Preferred PAM sequences for nucleases SEQ ID NOs: 20 and 26 are listed in Table 10. Values are calculated based on Walton et al. and PAM preferences are listed in order of preference (top of each list representing the more preferred sequences).

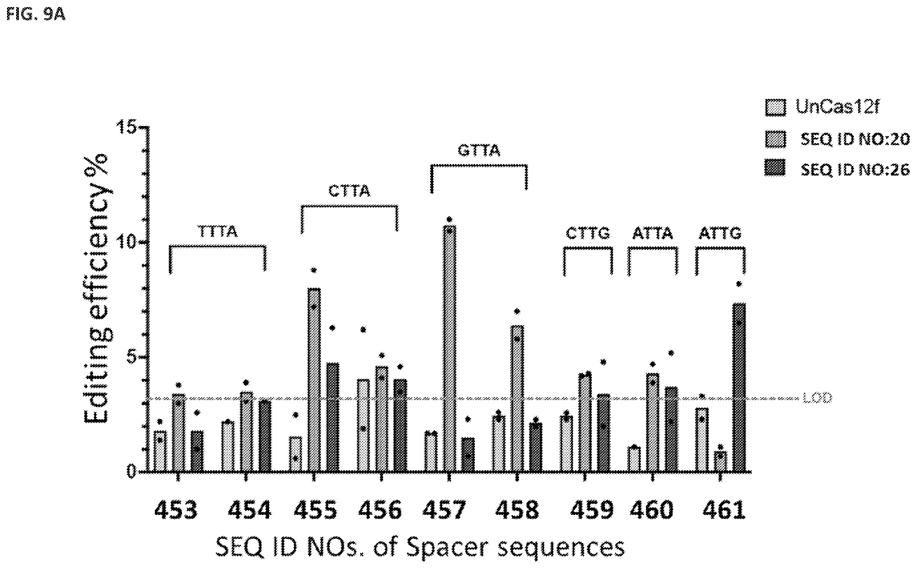

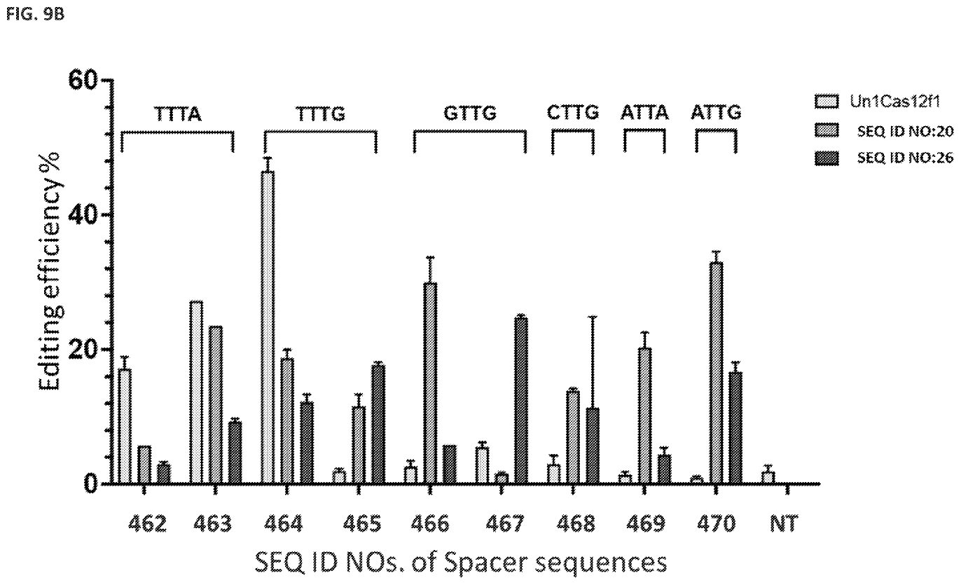

The identified PAM sequences were tested for editing activity with nucleases SEQ ID NOs: 20 and 26 in the context with a number of spacers in the sgRNAs. Results are shown in FIGS. 9A and 9B for target sequences (X-axis) with a higher level of editing (FIG. 9A) and target sequences with editing at a lower level (FIG. 9B) in combination with the various PAM sequences (PAM sequences shown above the bars by brackets). Surprisingly, the nucleases have a distinct PAM preference from that of known Cas12f nucleases such as Un1Cas12f1, AsCas12f, and SpaCas12f1. For the tested nucleases (SEQ ID NOs: 20, 21 and 26), the preferred PAM sequence was DTTR in which D is A, G or T and R is A or G; with a stronger bias towards ATTA PAMs. In contrast, for Un1Cas12f1 and AsCas12f, the PAM preference is TTTR and for SpaCas12f1, the PAM preference is NTTY in which N can be any base.

### Example 11

### AAV Vector Design and Editing in Mammalian Cells

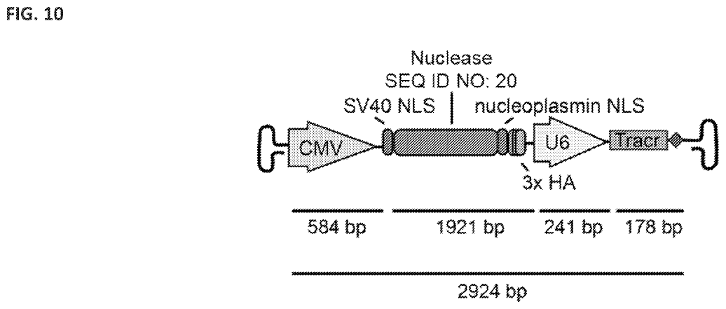

A single AAV vector was designed to deliver a nuclease of SEQ ID NO: 20 and sgRNA to mammalian cells using a CMV promoter and SV40 nuclear localization sequence at the 5′ end for the nuclease and a HA tag and nucleoplasmin localization sequence at 3′ end, followed by a U6 promoter for driving the expression of the sgRNA (shown as Tracr in FIG. 10). A representation of the vector is shown in FIG. 10.

Using this vector design, a set of constructs with the same nuclease but with different sgRNAs designed for different targets were constructed as shown in Table 11.

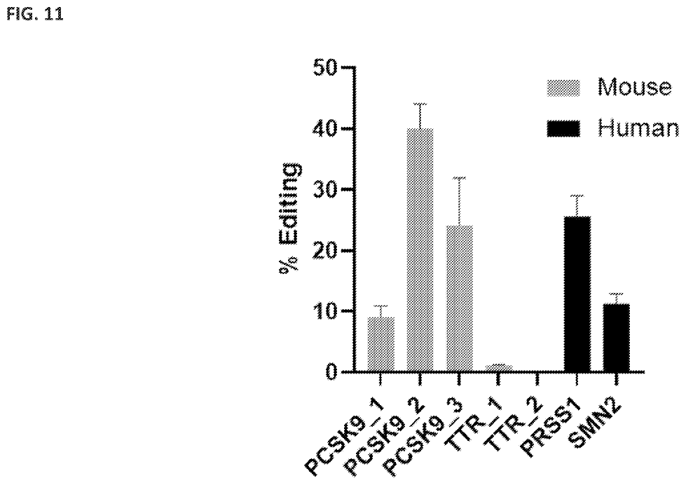

Constructs for human targets were tested in HEK293T cells and constructs for mouse targets were tested in NIH3T3 cells. Cells were plated at day 0 at a confluency of 3×10⁵ cells/m. At day 1, cells were transduced at 100K MOI. At day 2, etoposide (to enhance AAV delivery) was added to the cells to a final concentration of 60 mM and at day 3 cells were imaged. Cells were incubated for 72 hours and then were harvested following the methods of Example 2. Following DNA extraction, samples were prepared for NGS by amplifying each region with NGS specific primers listed on Table 12. NGS reads were processed using the CRISPRESSO2 tool (Clement, Kendell, et al. Nature biotechnology 37.3 (2019): 224-226, incorporated herein by reference in its entirety). Editing data for each construct is shown in FIG. 11.

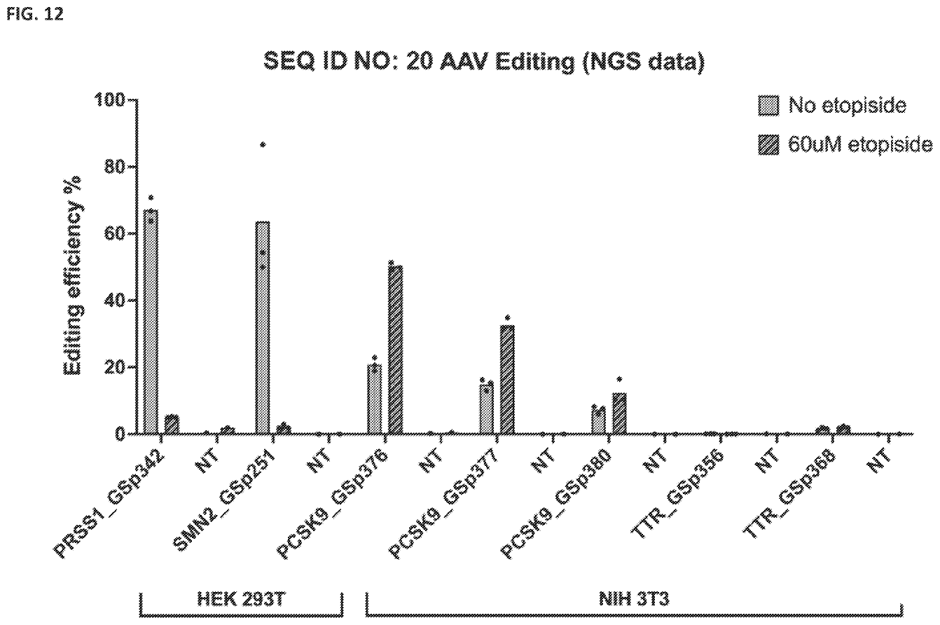

The SMN2 and TTR constructs were further tested with and without etoposide treatment for editing in HEK293T cells and NIH3T3 cells. Following the methods above, but with a MOI of 10K, cells were treated with etoposide was added on day 1, the AAV vector was added on day 2 and cells were harvested on day 7. Samples were prepared for NGS using primers from Table 9. NGS paired reads were processed using CRISPRESSO2 (Clement et al., 2019). Editing efficiencies are shown in FIG. 12. NIH3T3 cells were tolerant of the etoposide treatment and generally, editing was improved in the treated cells. In contrast, the HEK293T cells showed signs of toxicity and editing was reduced in the treated cells as compared to the cells that were not treated with etoposide.

TABLE 2

| sgRNA | | Corresponding |
|---|---|---|
| SEQ ID | | Nuclease |
| NO: | sgRNA sequence | SEQ ID NO: |
| 310 | TTGAAATAAAATGAATTTCAAACCCCTTCGGGGGTGGGCGTGTTGGAGCGCCTTAATTT | 21 |
| | GAGGTGCAGAATCCAAAAACTGCGACGATGTAGGTCGTTTCAGTCTCTGCGCACTCAAA | |
| | AAATTCACTTGATTaTTCAAGTGAATATCCAAC | |
| 344 | TTGAAATAAAATGAATTTCAAACCCCTTCGGGGGTGGGCGTGTTGGAGCGCCTTAATTT | 21 |
| | GAGGTGCAGAATCCAAAAACTGCGACGATGTAGGTCGTTTCAGTCTCTGCGCACTCAAA | |
| | AAATTCACTTGATTaTTTCGATAGTTGTAACTACCTTGAATTTCAAGTGAATATCCAAC | |
| 345 | TTGAAATAAAATGAATTTCAAACCCCTTCGGGGGTGGGCGTGTTGGAGCGCCTTAATTT | 21 |
| | GAGGTGCAGAATCCAAAAACTGCGACGATGTAGGTCGTTTCAGTCTCTGCGCACTCAAA | |
| | AAATTCACTTGATTaTTTCGATAgaaaTACCTTGAATTTCAAGTGAATATCCAAC | |
| 346 | AACCCCTTCGGGGGGGGCGTGTTGGAGCGCCTTAATTTGAGGTGCAGAATCCAAAAAC | 21 |
| | TGCGACGATGTAGGTCGTTTCAGTCTCTGCGCACTCAAAAAATTCACTTGATTaTTCAAG | |
| | TGAATATCCAAC | |
| 347 | TTGAAATAAAATGAATTTCAAACCCCTTCGGGGGGGGCGTGTTGGAGCGCCTTAATTT | 21 |
| | GAGGTGCAGAATCCTCTGCGCACTCAAAAAATTCACTTGATTaTTCAAGTGAATATCCAA | |
| | C | |
| 348 | AACCCCTTCGGGGGTGGGCGTGTTGGAGCGCCTTAATTTGAGGTGCAGAATCCTCTGCG | 21 |
| | CACTCAAAAAATTCACTTGATTaTTCAAGTGAATATCCAAC | |
| 349 | TTGAAATAAAATGAATTTCAAACCCCTTCGGGGGGGGCGTGTTGGAGCGCCTTAATTT | 21 |
| | GAGGTGCAGAATCCTCTGCGCACTCAAAAAATTCACTTGATTaTTTCGATAgaaaTACCTTG | |
| | AATTTCAAGTGAATATCCAAC | |
| 313 | ATTAAACCCCATTATGGGGTGGGCGTGTTGGAACGCCTTAGTTTGAGGTTTGAAAAACA | 24 |
| | AATTTGGGTTATATTTGGTAATCTTAATGTTCAAGCACTCAAAAAATTCACTTAAATTAaT | |
| | TTAAGTGGATATCCAAC | |
| 350 | ATTAAACCCCATTATGGGGGGGCGTGTTGGAACGCCTTAGTTTGAGGTTTGAAAAACA | 24 |
| | AATTTGGGTTATATTTGGTAATCTTAATGTTCAAGCACTCAAAAAATTCACTTAAAaTTTA | |
| | CAATGGTGTAAGCATCATGAAaTTTAAGTGGATATCCAAC | |
| 351 | ATTAAACCCCATTATGGGGTGGGCGTGTTGGAACGCCTTAGTTTGAGGTTTGAAAAACA | 24 |
| | AATTTGGGTTATATTTGGTAATCTTAATGTTCAAGCACTCAAAAAATTCACTTAAAaTTTA | |
| | CAATgaaaATCATGAAaTTTAAGTGGATATCCAAC | |
| 352 | TGGGCGTGTTGGAACGCCTTAGTTTGAGGTTTGAAAAACAAATTTGGGTTATATTTGGTA | 24 |
| | ATCTTAATGTTCAAGCACTCAAAAAATTCACTTAAATTAaTTTAAGTGGATATCCAAC | |
| 353 | ATTAAACCCCATTATGGGGTGGGCGTGTTGGAACGCCTTAGTTTGAGGTTTGAAAAACA | 24 |
| | AATATGTTCAAGCACTCAAAAAATTCACTTAAATTAaTTTAAGTGGATATCCAAC | |
| 354 | TGGGGTGGGCGTGTTGGAACGCCTTAGTTTGAGGTTTGAAAAACAAATATGTTCAAGCA | 24 |
| | CTCAAAAAATTCACTTAAATTAaTTTAAGTGGATATCCAAC | |
| 355 | ATTAAACCCCATTATGGGGTGGGCGTGTTGGAACGCCTTAGTTTGAGGTTTGAAAAACA | 24 |
| | AATATGTTCAAGCACTCAAAAAATTCACTTAAAaTTTACAATgaaaATCATGAAaTTTAAGT | |
| | GGATATCCAAC | |
| 325 | CTTTCGGGATGGGCGCGTTGGAGCGCCTTGGTTTGAGGTGAGGACACCATAATCCGCAT | 36 |
| | AATGAATATTGTACGGATGTCCCTGCACTCGAAAAGTTCACTTGATTaTCAAGTGAATAT | |
| | CCAAC | |
| 356 | CTTTCGGGATGGGCGCGTTGGAGCGCCTTGGTTTGAGGTGAGGACACCATAATCCGCAT | 36 |
| | AATGAATATTGTACGGATGTCCCTGCACTCGAAAAGTTCACTTGATaTTGTTGTAACTGC | |
| | GTTGATTaTCAAGTGAATATCCAAC | |
| 357 | CTTTCGGGATGGGCGCGTTGGAGCGCCTTGGTTTGAGGTGAGGACACCATAATCCGCAT | 36 |
| | AATGAATATTGTACGGATGTCCCTGCACTCGAAAAGTTCACTTGATTTaTTTCAAGTGAA | |
| | TATCCAAC | |
| 358 | TGGGCGCGTTGGAGCGCCTTGGTTTGAGGTGAGGACACCATAATCCGCATAATGAATAT | 36 |
| | TGTACGGATGTCCCTGCACTCGAAAAGTTCACTTGATTaTCAAGTGAATATCCAAC | |
| 359 | CTTTCGGGATGGGCGCGTTGGAGCGCCTTGGTTTGAGGTGAGGACACCATGTCCCTGCA | 36 |
| | CTCGAAAAGTTCACTTGATTATCAAGTGAATATCCAAC | |
| 360 | CTTTCGGGATGGGCGCGTTGGAGCGCCTTGGTTTGAGGTGAGGACACCATGTCCCTGCA | 36 |
| | CTCGAAAAGTTCACTTGATaTTGTTGTAACTGCGTTGATTaTCAAGTGAATATCCAAC | |
| 361 | CCTTCGGGGGTGGGCGTGTTGGAGCGCCTTAATTTGAGGTGCAGAATCCAAAAACTGCG | 21 |
| | ACGATGTAGGTCGTTTCAGTCTCTGCGCACTCAAAAAATTCACTTGATTaTTCAAGTGAA | |
| | TATCCAAC | |
| 362 | CCCCTTCGGGGGTGGGCGTGTTGGAGCGCCTTAATTTGAGGTGCAGAATCCAAAAACTG | 21 |
| | CGACGATGTAGGTCGTTTCAGTCTCTGCGCACTCAAAAAATTCACTTGATTaTTCAAGTG | |
| | AATATCCAAC | |
| 363 | CAAACCCCTTCGGGGGTGGGCGTGTTGGAGCGCCTTAATTTGAGGTGCAGAATCCAAAA | 21 |
| | ACTGCGACGATGTAGGTCGTTTCAGTCTCTGCGCACTCAAAAAATTCACTTGATTaTTCA | |
| | AGTGAATATCCAAC | |
| 364 | TTCAAACCCCTTCGGGGGGGGCGTGTTGGAGCGCCTTAATTTGAGGTGCAGAATCCAA | 21 |
| | AAACTGCGACGATGTAGGTCGTTTCAGTCTCTGCGCACTCAAAAAATTCACTTGATTaTT | |
| | CAAGTGAATATCCAAC | |
| 365 | ATTTCAAACCCCTTCGGGGGTGGGCGTGTTGGAGCGCCTTAATTTGAGGTGCAGAATCC | 21 |
| | AAAAACTGCGACGATGTAGGTCGTTTCAGTCTCTGCGCACTCAAAAAATTCACTTGATTa | |
| | TTCAAGTGAATATCCAAC | |
| 366 | GAATTTCAAACCCCTTCGGGGGTGGGCGTGTTGGAGCGCCTTAATTTGAGGTGCAGAAT | 21 |
| | CCAAAAACTGCGACGATGTAGGTCGTTTCAGTCTCTGCGCACTCAAAAAATTCACTTGAT | |
| | TaTTCAAGTGAATATCCAAC | |
| 367 | CGTGTTGGAACGCCTTAGTTTGAGGTTTGAAAAACAAATTTGGGTTATATTTGGTAATCT | 24 |
| | TAATGTTCAAGCACTCAAAAAATTCACTTAAATTAaTTTAAGTGGATATCCAAC | |
| 368 | GGCGTGTTGGAACGCCTTAGTTTGAGGTTTGAAAAACAAATTTGGGTTATATTTGGTAAT | 24 |
| | CTTAATGTTCAAGCACTCAAAAAATTCACTTAAATTAaTTTAAGTGGATATCCAAC | |
| 369 | GGTGGGCGTGTTGGAACGCCTTAGTTTGAGGTTTGAAAAACAAATTTGGGTTATATTTGG | 24 |
| | TAATCTTAATGTTCAAGCACTCAAAAAATTCACTTAAATTAaTTTAAGTGGATATCCAAC | |
| 370 | GGGGTGGGCGTGTTGGAACGCCTTAGTTTGAGGTTTGAAAAACAAATTTGGGTTATATTT | 24 |
| | GGTAATCTTAATGTTCAAGCACTCAAAAAATTCACTTAAATTAaTTTAAGTGGATATCCA | |
| | AC | |
| 371 | ATGGGGTGGGCGTGTTGGAACGCCTTAGTTTGAGGTTTGAAAAACAAATTTGGGTTATA | 24 |
| | TTTGGTAATCTTAATGTTCAAGCACTCAAAAAATTCACTTAAATTAaTTTAAGTGGATAT | |
| | CCAAC | |
| 372 | TTATGGGGTGGGCGTGTTGGAACGCCTTAGTTTGAGGTTTGAAAAACAAATTTGGGTTAT | 24 |
| | ATTTGGTAATCTTAATGTTCAAGCACTCAAAAAATTCACTTAAATTAaTTTAAGTGGATA | |
| | TCCAAC | |
| 373 | ATATATGTAACTATATAATCCCTTTCGGGATGGGCGCGTTGGAGCGCCTTGGTTTGAGGT | 36 |
| | GAGGACACCATAATCCGCATAATGAATATTGTACGGATGTCCCTGCACTCGAAAAGTTC | |
| | ACTTGATTaTCAAGTGAATATCCAAC | |
| 374 | AATCCCTTTCGGGATGGGCGCGTTGGAGCGCCTTGGTTTGAGGTGAGGACACCATAATC | 36 |
| | CGCATAATGAATATTGTACGGATGTCCCTGCACTCGAAAAGTTCACTTGATTaTCAAGTG | |
| | AATATCCAAC | |
| 375 | ATAATCCCTTTCGGGATGGGCGCGTTGGAGCGCCTTGGTTTGAGGTGAGGACACCATAA | 36 |
| | TCCGCATAATGAATATTGTACGGATGTCCCTGCACTCGAAAAGTTCACTTGATTaTCAAG | |
| | TGAATATCCAAC | |
| 376 | GGCGCGTTGGAGCGCCTTGGTTTGAGGTGAGGACACCATAATCCGCATAATGAATATTG | 36 |
| | TACGGATGTCCCTGCACTCGAAAAGTTCACTTGATTaTCAAGTGAATATCCAAC | |
| 377 | GATGGGCGCGTTGGAGCGCCTTGGTTTGAGGTGAGGACACCATAATCCGCATAATGAAT | 36 |
| | ATTGTACGGATGTCCCTGCACTCGAAAAGTTCACTTGATTaTCAAGTGAATATCCAAC | |
| 378 | GGGATGGGCGCGTTGGAGCGCCTTGGTTTGAGGTGAGGACACCATAATCCGCATAATGA | 36 |
| | ATATTGTACGGATGTCCCTGCACTCGAAAAGTTCACTTGATTaTCAAGTGAATATCCAAC | |
| 379 | TTTGATATAGGATGTATATACGAATTTCAATTACCACCCCAATGGGGTGAGGGCGTGTTG | 19 |
| | GAGCGCCTTAGTTTGAGGTTTGATACTAAAAATTGAGATGATGGAGGTCATTTCGATAA | |
| | TCAAGCACTCAAAA | |
| 380 | TGGGCGTGTTGGAACGCCTTAGTTTGAGGTCAGGATTAAAAAATTGACAAGACGCAGGT | 20 |
| | CTATTCAGTACCGTGGCACTCAAAAAATTCACTTGATTaTaTCAAGTGAATATCCAAC | |
| 381 | ATATGAATTTCATTGCCCATTaTGGGCTGGGCGTGTTGGAACGCCTTAGTTTGAGGTCTG | 22 |
| | AAAATGAAAATTGTGGTTGCATAGGCACTCTCGATATTCAAGaaagGGTGTTAATGCCTTG | |
| 382 | GTGAATATCCAATAATAGATATAATGGATTTCAAGTCCCTTCGGGGACGGGCGTGTTGG | 23 |
| | AACGCCTTAGTTTGAGGTTTGGATTC | |
| 383 | GTTGGAACGGCCTCAATTaTGAGGCTTAGCCTTAGTTTGAGGTTTGGATTCAAAAAATCG | 25 |
| | TTGGTGTGTAGGCACTTTCGATTTCCAAGCACTCAAAAAATTCACTTATAAGTGAATATC | |
| | CAAC | |
| 384 | TGAATTTCAATTCCCCTCTGGGGGAAGGGCGTGTTGGAACGCCTTAGTTTGAGGTTTGAA | 26 |
| | AATGAAAAATTGGGTGGTGTGGAGGCACTCCCAATaaagGATGTTATCGGATATCCAAC | |
| 385 | TGAATTTCATTACCCATTaTGGGGTGGGCGTGTTGGAACGCCTTAGTTTGAGGTTTGAAA | 27 |
| | ACAGAAATTAGGATTGCGGAGGCATTCTTGATGTTCA | |
| 386 | aTGGGCTGGGCGTGTTGGAACGCCTTAGTTTGAGGTTTGAAAACGAAAATTGGGAATGT | 28 |
| | AGAGGCACTCTCGATATTCAAGaaagCTTGAATT | |
| 387 | TGGGCGTGTTGGAACGCCTTAGTTTGAGGTTTGAAGATAAAAATCAAATTGGTGGAGGC | 29 |
| | CTTTGATATTCAAGCACTCAAAAAATTCACTTATTTGTGATATATAGTTGGAAATCAACA | |
| | CATAGTGGATATCCAAC | |
| 388 | GTTCTTTGAAATATATAGATATGGATTTCAATTTCCCGTTTATGGGATGGGCGTGTTGGA | 34 |
| | ACGCCTTAaaagGGTGTATTTGCCTATGTATTTAAGTGGATATCCAAC | |
| 389 | AACCCCATTATGGGGTGGGCGTGTTGGAACGCCTTAGTTTGAGGTTTGAAAAACAAATT | 24 |
| | TGGGTTATATTTGGTAATCTTAATGTTCAAGCACTCAAAAAATTCACTTAAATTAaTTTAA | |
| | GTGGATATCCAAC | |
| 390 | ATTAAACCCCATTATGGGGTGGGCGTGTTGGAACGCCTTAGTTTGAGGTGCAGAATCCA | 24 |
| | AAAACTGCGACGATGTAGGTCGTTTCAGTCTCTGCGCACTCAAAAAATTCACTTAAATTA | |
| | aTTTAAGTGGATATCCAAC | |
| 391 | ATTAAACCCCTTATGGGGTGGGCGTGTTGGAACGCCTTAGTTTGAGGTTTGAAAAACAA | 24 |
| | ATTTGGGTTATATTTGGTAATCTTAATGTTCAAGCACTCAAAAAATTCACTTAgATTATTC | |
| | TAAGTGGATATCCAAC | |
| 392 | AACCCCTTATGGGGTGGGCGTGTTGGAACGCCTTAGTTTGAGGTGCAGAATCCAAAAAC | 24 |
| | TGCGACGATGTAGGTCGTTTCAGTCTCTGCGCACTCAAAAAATTCACTTAgATTAITcTAA | |
| | GTGGATATCCAAC | |
| 393 | AACCCCTTCGGGAATGGGCGTGTTGGAACGCCTTAGTTTGAGGTCAGGATTAAAAAATT | 20 |
| | GACAAGACGCAGGTCTaTTCAGTACCGTGGCACTCAAAAAATTCACTTGATTaTaTCAAGT | |
| | GAATATCCAAC | |
| 394 | CTTCGGGggTGGGCGTGTTGGAACGCCTTAGTTTGAGGTCAGGATTAAAAAATTGACAAG | 20 |
| | ACGCAGGTCTaTTCAGTACCGTGGCACTCAAAAAATTCACTTGATTaTaTCAAGTGAATAT | |
| | CCAAC | |
| 395 | AACCCCTTCGGGggTGGGCGTGTTGGAACGCCTTAGTTTGAGGTCAGGATTAAAAAATTG | 20 |
| | ACAAGACGCAGGTCTaTTCAGTACCGTGGCACTCAAAAAATTCACTTGATTaTaTCAAGTG | |
| | AATATCCAAC | |
| 396 | ATTCCCCTCTGGGGGAAGGGCGTGTTGGAACGCCTTAGTTTGAGGTTTGAAAATGAAAA | 26 |
| | ATTGGGTGGTGTGGAGGCACTCCCAATaaagGATGTTATCGGATATCCAAC | |
| 397 | AACCCCTtcGGGGGAGGGCGTGTTGGAACGCCTTAGTTTGAGGTGCAGAATCCAAAAACT | 26 |
| | GCGACGATGTAGGTCGTTTCAGTCTCGGCACaCtCAAaaaaTTCACTTgGATaTTcaATCGG | |
| | ATATCCAAC | |
| 398 | TGCCCATTaTGGGCTGGGCGTGTTGGAACGCCTTAGTTTGAGGTgCTGAAAATGAAAATT | 22 |
| | GTGGTTGCATAGGCACTCTCGATCTCTGCGCATTCAAGaaaTTAACTTGAGTaTTcAAGTGA | |
| | ATATCCAAC | |
| 399 | TGCCCATTaTGGGCTGGGCGTGTTGGAACGCCTTAGTTTGAGGTCTGAAAATGAAAATTG | 22 |
| | TGGTTGCATAGGCACTCTCGATATTCAAGaaagGGTGTTAATGCCTTGAGTaTTAAGTG | |
| 400 | AACCCCTTCGGGGTACGGGCGTGTTGGAACGCCTTAGTTTGAGGTTTGGATTCAAAAAA | 25 |
| | TCGTTGGTGTGTAGGCACTTTCGATTTCCAAGCACTCAAAAAATTCACTTATAAGTGAAT | |
| | ATCCAAC | |
| 401 | TACCCATTaTGGGGTGGGCGTGTTGGAACGCCTTAGTTTGAGGTTTGAAAACAGAAATTA | 27 |
| | GGATTGCGGAGGCATTCTTGATGTTCAAGCAaaagAGTGTTAATGCTTTGAC | |
| 402 | AGCCTATTaTGGGCTGGGCGTGTTGGAACGCCTTAGTTTGAGGTTTGAAAACGAAAATTG | 28 |
| | GGAcgATGTAGATCGTTTCAGTCTCGGCACTCTCGAAAAATTCACTTGATTATTCAAGaaag | |
| | CTTGAATT | |
| 403 | AACCCATTATGGGTTGGGCGTGTTGGAACGCCTTAGTTTGAGGTTTGAAGATAAAAATC | 29 |
| | AAATTGGTGGAGGCCTTTGATATTCAAGCACTCAAAAAATTCACTTATTTGTTCAAGTGG | |
| | ATATCCAAC | |
| 404 | AACCCCTTCGGGGGGGGCGTGTTGGAGCGCCTTAAGTGCAGAATCCAAAAACTGCGAC | 21 |
| | GATGTAGGTCGTTTCAGTCTCTGCGCACTCAAAAAATTCACTTGATTaTTCAAGTGAATA | |
| | TCCAAC | |
| 405 | AACCCCTTCGGGGGTGGGCGTGTTGGAGCGCCTTAATTTGAGGTGCAGAATCCAAAAAC | 21 |
| | TGCGACGATGTAGGTCGTTTCAGTCTCTGCGCAAAATTCACTTGATTaTTCAAGTGAATA | |
| | TCCAAC | |
| 406 | AACCCCTTCGGGGGTGGGCGTGTTGGAGCGCCTTAAGTGCAGAATCCAAAAACTGCGAC | 21 |
| | GATGTAGGTCGTTTCAGTCTCTGCGCAAAATTCACTTGATTaTTCAAGTGAATATCCAAC | |
| 407 | AACCCCTTCGGGGGTGGGCGTGTTGGAGCGCCTTAATTTGAGAATCCAAAAACTGCGAC | 21 |
| | GATGTAGGTCGTTTCAGTCTCTGCGCACTCAAAAAATTCACTTGATTaTTCAAGTGAATA | |
| | TCCAAC | |
| 408 | AACCCCTTCGGGGGTGGGCGTGTTGGAGCGCCTTAATTTGAGGTGCAGAATCCAAAAAC | 21 |
| | TGCGACGATGTAGGTCGTTTCAGTCTACTCAAAAAATTCACTTGATTaTTCAAGTGAATA | |
| | TCCAAC | |
| 409 | AACCCCTTCGGGGGTGGGCGTGTTGGAGCGCCTTAATTTGAGAATCCAAAAACTGCGAC | 21 |
| | GATGTAGGTCGTTTCAGTCTACTCAAAAAATTCACTTGATTaTTCAAGTGAATATCCAAC | |
| 410 | AACCCCTTCGGGGGGGGCGTGTTGGAGCGCCTTAATTTGAGGTGCAGCTGCGACGATG | 21 |
| | TAGGTCGTTTCAGTCTCTGCGCACTCAAAAAATTCACTTGATTATTCAAGTGAATATCCA | |
| | AC | |
| 411 | AACCCCTTCGGGGGGGGCGTGITGGAGCGCCTTAATTTGAGGTGCAGAATCCAAAAAC | 21 |
| | TGCGACGATGTAGGTCGTTTCAGCTGCGCACTCAAAAAATTCACTTGATTaTTCAAGTGA | |
| | ATATCCAAC | |
| 412 | AACCCCTTCGGGGGTGGGCGTGTTGGAGCGCCTTAATTTGAGGTGCAGCTGCGACGATG | 21 |
| | TAGGTCGTTTCAGCTGCGCACTCAAAAAATTCACTTGATTaTTCAAGTGAATATCCAAC | |
| 413 | AACCCCTTCGGGGGTGGGCGTGTTGGAGCGCCTTAATTTGAGGTGCAGAATCCAAAAAA | 21 |
| | CGATGTAGGTCGTTTCAGTCTCTGCGCACTCAAAAAATTCACTTGATTaTTCAAGTGAAT | |
| | ATCCAAC | |
| 414 | AACCCCTTCGGGGGTGGGCGTGTTGGAGCGCCTTAATTTGAGGTGCAGAATCCAAAAAC | 21 |
| | TGCGACGATGTAGGTCGTTCTCTGCGCACTCAAAAAATTCACTTGATTaTTCAAGTGAAT | |
| | ATCCAAC | |
| 415 | AACCCCTTCGGGGGTGGGCGTGTTGGAGCGCCTTAATTTGAGGTGCAGAATCCAAAAAA | 21 |
| | CGATGTAGGTCGTTCTCTGCGCACTCAAAAAATTCACTTGATTaTTCAAGTGAATATCCA | |
| | AC | |
| 416 | AACCCCTTCGGGGGTGGGCGTGTTGGAGCGCCTTAATTTGAGGTGCAGAATCCAAAAAC | 21 |
| | TGCGTGTAGGTCGTTTCAGTCTCTGCGCACTCAAAAAATTCACTTGATTaTTCAAGTGAA | |
| | TATCCAAC | |
| 417 | AACCCCTTCGGGGGTGGGCGTGTTGGAGCGCCTTAATTTGAGGTGCAGAATCCAAAAAC | 21 |
| | TGCGACGATGTAGGTTCAGTCTCTGCGCACTCAAAAAATTCACTTGATTaTTCAAGTGAA | |
| | TATCCAAC | |
| 418 | AACCCCTTCGGGGGGGGCGTGTTGGAGCGCCTTAATTTGAGGTGCAGAATCCAAAAAC | 21 |
| | TGCGTGTAGGTTCAGTCTCTGCGCACTCAAAAAATTCACTTGATTaTTCAAGTGAATATC | |
| | CAAC | |
| 419 | AACCCCTTCGGGGGTGGGCGTGTTGGAGCGCCTTAATTTGAGGTGCAGAATCCAAAAAC | 21 |
| | TGCGACGATCGTTTCAGTCTCTGCGCACTCAAAAAATTCACTTGATTaTTCAAGTGAATA | |
| | TCCAAC | |
| 420 | GGGCGTGTTGGAGCGCCTTAATTTGAGGTGCAGAATCCAAAAACTGCGACGATGTAGGT | 21 |
| | CGTTTCAGTCTCTGCGCACTCAAAAAATTCACTTGATTaTTCAAGTGAATATCCAAC | |
| 421 | AACCCCTTCGGGGGTGGGCGTGTTGGAGCGCCTTAATTTGAGGTGCAGAATCCAAAAAC | 21 |
| | TGCGACGATGTAGGTCGTTTCAGTCTCTGCGCACTCAAAAAATTCACTTGATTaTTCAAG | |
| | TGAATA | |
| 422 | GGGCGTGTTGGAGCGCCTTAATTTGAGGTGCAGAATCCAAAAACTGCGACGATGTAGGT | 21 |
| | CGTTTCAGTCTCTGCGCACTCAAAAAATTCACTTGATTaTTCAAGTGAATA | |
| 472 | AACCCCTTCGGGggTGGGCGTGTTGGAACGCCTTAGTTTGAGGTCAGGATTAAAAAATTG | 20 |
| | ACAAGACGCAGGTCTaTTCAGTACCTGGCACTCAAAAAATTCACTTGATTaTaTCAAGTGA | |
| | ATATCCAAC | |
| 473 | AACCCCTTCGGGggTGGGCGTGTTGGAACGCCTTAGTTTGAGGTCAGGATTAAAAAACTG | 20 |
| | ACAAGACGCAGGTCTaTTCAGTACCTGGCACTCAAAAAATTCACTTGATTaTaTCAAGTGA | |
| | ATATCCAAC | |
| 474 | AACCCCTTCGGGggTGGGCGTGTTGGAACGCCTTAGTTTGAGGTCAGGATTAAAAAACTG | 20 |
| | CAAGACGCAGGTCTaTCAGTACCTGGCACTCAAAAAATTCACTTGATTaTaTCAAGTGAAT | |
| | ATCCAAC | |
| 475 | AACCCCTTCGGGggTGGGCGTGTTGGAACGCCTTAGTTTGAGGTCAGGATTAAAAAACTG | 20 |
| | CgAGACGCAGGTCTTTCAGTACCTGGCACTCAAAAAATTCACTTGATTaTaTCAAGTGAAT | |
| | ATCCAAC | |
| 476 | AACCCCTTCGGGggTGGGCGTGTTGGAACGCCTTAGTTTGAGGTCAGGATTAAAAAACTG | 20 |
| | CgAcGACGCAGGTCgTTTCAGTACCTGGCACTCAAAAAATTCACTTGATTaTaTCAAGTGA | |
| | ATATCCAAC | |
| 477 | AACCCCTTCGGGggTGGGCGTGTTGGAACGCCTTAGTTTGAGGTgCAGGATTAAAAAACT | 20 |
| | GCgAcGACGCAGGTCgTTTCAGTACCTGcGCACTCAAAAAATTCACTTGATTaTaTCAAGTG | |
| | AATATCCAAC | |
| 478 | AACCCCTTCGGGggTGGGCGTGTTGGAACGCCTTAGTTTGAGGTgCAGGATTAAAAAACT | 20 |
| | GCgAcGACGCAGGTCgTTTCAGTACCTGcGCACTCAAAAAATTCACTTGATTaTTCAAGTG | |
| | AATATCCAAC | |
| 479 | AACCCCTTCGGGGGGGGCGTGTTGGAGCGCCTTAATTTGAGGTGCAGAATCCAAAAAC | 21 |
| | TGCGACGATGTAGGTCGTTTCAGTCTCTGCGCACTCAAAAAATTCACTTGATTaTTTCGAT | |
| | AgaaaTACCTTGAATTTCAAGTGAATATCCAAC | |
| 480 | AACCCCTTCGGGGGTGGGCGTGTTGGAGCGCCTTAATTTGAGGTGCAGAATCCAAAAAC | 21 |
| | TGCGACGATGTAGGTCGTTTCAGTCTCTGCGCACTCAAAAAATTCACTTGATTaTTTCGAT | |
| | AGTTGTAACTACCTTGAATTTCAAGTGAATATCCAAC | |
| 481 | AACCCCTTCGGGGGGGGCGTGTTGGAGCGCCTTAAcgcGAGGTGCAGAATCCAAAAACT | 21 |
| | GCGACGATGTAGGTCGTTTCAGTCTCTGCGCACTCgcgAAATTCACTTGATTaTTCAAGTG | |
| | AATATCCAAC | |
| 482 | AACCCCTTCGGGGGGGGCGTGTTGGAGCGCCTTAAcgcGAGGTGCAGAATCCAAAAACT | 21 |
| | GCGACGATGTAGGTCGTTTCAGTCTCTGCGCCTCgcgAAATTCACTTGATTaTTCAAGTGA | |
| | ATATCCAAC | |

TABLE 3

| Bar | Nuclease | sgRNA |
|---|---|---|
| 1 | SEQ ID NO: 21 | SEQ ID NO: 310 |
| 2 | SEQ ID NO: 21 | SEQ ID NO: 346 |
| 3 | SEQ ID NO: 21 | SEQ ID NO: 347 |
| 4 | SEQ ID NO: 21 | SEQ ID NO: 348 |
| 5 | SEQ ID NO: 21 | SEQ ID NO: 345 |
| 6 | SEQ ID NO: 21 | SEQ ID NO: 349 |
| 7 | SEQ ID NO: 21 | SEQ ID NO: 313 |
| 8 | SEQ ID NO: 21 | SEQ ID NO: 325 |
| 9 | SEQ ID NO: 24 | SEQ ID NO: 313 |
| 10 | SEQ ID NO: 24 | SEQ ID NO: 352 |
| 11 | SEQ ID NO: 24 | SEQ ID NO: 353 |
| 12 | SEQ ID NO: 24 | SEQ ID NO: 354 |
| 13 | SEQ ID NO: 24 | SEQ ID NO: 351 |
| 14 | SEQ ID NO: 24 | SEQ ID NO: 355 |
| 15 | SEQ ID NO: 24 | SEQ ID NO: 310 |
| 16 | SEQ ID NO: 24 | SEQ ID NO: 325 |
| 17 | SEQ ID NO: 36 | SEQ ID NO: 325 |
| 18 | SEQ ID NO: 36 | SEQ ID NO: 358 |
| 19 | SEQ ID NO: 36 | SEQ ID NO: 359 |
| 20 | SEQ ID NO: 36 | SEQ ID NO: 356 |
| 21 | SEQ ID NO: 36 | SEQ ID NO: 357 |
| 22 | SEQ ID NO: 36 | SEQ ID NO: 360 |
| 23 | SEQ ID NO: 36 | SEQ ID NO: 310 |
| 24 | SEQ ID NO: 36 | SEQ ID NO: 313 |

TABLE 4

| SEQ ID | |
|---|---|
| NO | Nuclease Amino Acid Sequence |
| 1 | MTKVIKLALICQQSDSNGMPVDYKEVNKILWELQRQTREIKNKSIQYCWEYHNFSSDYYKRNGEY |
| | PKEKDVLLFTLGGYVNDKFKTGNDLYSANCSTTVRGVCGEFKNSKKDFISGKRSIISYKENQPLD |
| | LHNKSIRLEYSDHEFYVYLKLLNRQGFKKFNFADTQIMFKILVRDNSTKTILERCLDEVYSVSAS |
| | KLIYDKKKKCWVLNLSYSFSGEITHNLDENRILGVDLGIHYPICASVYGEWKRFTIDGGEIEEYR |
| | RRVEARKKTLLKQGKNCGDGRIGHGVKTRNKPVYSIEDRISRFRDTANHKYSRALINYAIKNNCG |
| | VIQMENLEGVTAHSDKFLKNWSYYDLQTKIEYKAKEAGIKVVYINPRYTSQRCSKCGYTDTDNRP |
| | EQAKFICKKCGFSENADFNASQNIGIKNIEQIIKEEIQI |
| 2 | MQKVLKVYLICEQLDPDGSPVDYKDIFKLLWDLQKQTREIKNKSIQYCWEFSNFSSDYYKEHHEY |
| | PKDKEILNYTLGGFVNDKFKTGNDLYSANCSTTVRTACAEFKNAKADFMRGEKSIISYKANQPLD |
| | LHNKSIRLEYQKGTFLFYLKLLNRSAVKKHGFQSSEIRFKAIVKDNSSQTILERCTAGVYDMAAS |
| | KLLYDQKKKCWVLNLVYAFEPETPEALDPEKILGVDLGVHYPICASVYGDLKRFIIDGGEIESFR |
| | KRVEARKISMLKQGKNCGGGRIGHGIQTRNKPVYAIADKIARFRDTVNHKYSRALIDYAIKNNCG |
| | VIQMEKLTGVTADANRFMKNWTYFDLQTKIEYKAQEAGIQIVYIEPKYTSQRCSKCGYIDRENRP |
| | EQSKFICRKCGFSENADYNASQNIGIRNIEKLIEKQLQTKCESETDTT |
| 3 | MKKTVRLQIVKPMDEDWEILGRVLHDIRYQTRQVLNKTIQLCWEYSNFSSEYKALHGDYPKNKEI |
| | LHYTSMHGYAYNQLKEQYYYIQSGNCSQTVKRAVDKWKSDLKEILRGDRSIPSYKKDIPIDIVKD |
| | AVSLEHDEKNGNYIATLSLMSTAYRKEMERKSGQFRVLLHSGDNSKKTILKRLVQGEYQHTASQI |
| | VKKGKKWFLNLSYKFDVLQTPFQTERVMGVDMGVIYPIYMAFNYHDHLRYKIQGGEIERFRRQVE |
| | SRKKALQDQGKYAGSGRVGHGTKTRIDPLEVIRDKIANFRETKNHHYSRYVVDMAEKHECATIQL |
| | EELKGIHQDDAFLKRWSYHDLQEKITYKAEEKGIQVIKVDPQKTSQRCHHCGNIDSNNRKEQASF |
| | LCTSCGMETNADFNAAKNISIPGIEQIIQTEMKS |
| 4 | MCALTKIMKYELRYLDGFPDFSAMQNAVWPLQRQTREILNRTIQEAYRWDYFSATKKKETGEYPD |
| | LQKETGYKRLDGYIYHVLSPDYPDFSSSGVNATIQKAWKKYKSSKADVWKGEMSLPSYKSDQPIV |
| | LHAKQIKLSGDTRAAAATLSLFSNKFKKEHEISGNVQFAITLHDNTQRTIYQKLRNGEYKLSESQ |
| | LVYDKKKWFLYLAYSFNPAEHALDPEKILGVDMGEKFALYASSFGEYGHFKIEGSEVTEYAKALE |
| | RRRRSLQQQARYCGEGRIGHGTKTRVGVVYREEDRIANFRSTINHRYSKALIEYAVKNGYGTIQM |
| | ENLTGIKENLQFPRRLQHWTYYDLQSKIEAKAKEHGIAVVKVNPKHTSQRCSRCGHIAAENRPKQ |
| | EVFQCVKCGYACNADFNASQNISIKDIEKLIQETIGANPK |
| 5 | MVLTRKLQLVPCAEGMSKEEAKKEVDRVYKILRDGIYAQNKAYNIFISRRYTAILLGASKEELAK |
| | LTLIGERNPKKDDPSYSLYEYGKINFIKGIPQASALGQHAISDLSKQKKDGLFKGKVALACRRLD |
| | APMWIKQKYEFYHNYADNKELSENLYSEDLKIYMKLANICVFEVVLGNPHKSAAIRAELERVFDE |
| | NYKKLDSSIQIVNNKIMLYLAINIPEKQIELKEDVVVGVDLGIAIPAVCALNNSRYIKKSIGSAN |
| | EFLRIRTQLQSEKKRLQRKLEDINGGHGRKKKLAPLDKLSKRERNFVQTYNHMISRRVVDFAVKN |
| | NAKYINVEDLSDYKNNGSEYILRNWSYYELQQQIEYKAEMYGIVVRKVNPYHTSQICSQCGHWEE |
| | GQRKSQSEFECKACGYTANADFNAARNIALSTDFVKK |
| 6 | MSKGVVTKVMKYTLRYIGGCGDFHKMQEAVWKLQRQTREVLNKTVQLAFDWDHRSREAYRTSGEY |
| | LDIVKETGYKRLDGYIYNRLKTDYADFASSNLNATIQVAWKKYMASKSDILTGKMSFPSYKSNQP |
| | IVLHNSSIRFSTEKYGVPAAELTVFSNALKKENGLSTNPAFEILLMDGTQRSIFQRVISGEYKHG |
| | QCQINYEKRKWFLYLTYTFEAGKTPLDPDKILGVDIGETLAICASSTSEWGRFVIQGGEVTRYSK |
| | QIEERRRSQQRQATYCGEGRIGHGTKARVAPIYATEDRIANFRDTINHRYSKALIEYAVKHGFGT |
| | IQMEDLSGIKGEKDFPKFLRHWTYYDLQNKIEAKAKEQGINVVKVQPAYTSQRCSKCGCIDKENR |
| | KNQELFCCVKCGFKANADFNASQNISIKGIDKIIEKEYNANIE |
| 7 | MNKVVKLALISKVKDKDGNDVKYGDVCKILWELQRQTREIKNKAVQFCWEWNGFSSEYNNLFGEY |
| | PKDKDYLKNKSEGKPIVLRSFVYDRLKSDYYLNSSNLSTTTSLAFKEFKQYLTDIRKGERSVLNY |
| | KNNQPLELHNECIWLESNNGKFITRESFLNKAGKDFYSIDNFTFEVIVKDNSTKTILERCIDSIY |
| | GIRASKLIYNQKKKQWFLNLSYSFEAKEIATLDKDKILGVDLGIALPICASVYGDLDRFTIKGGE |
| | IEHFRKSIESRKRSLLQQGKVCGDGRIGHGIHTRNKPAYNIEDKIARFRDTANHKYSRALIDYAV |
| | RKGCGTIQMEMLKGITEEKDPFLKNWTYFDLQQKIEYKAKEKGIKVVYIAPKYTSRRCSKCGHID |
| | KDNRLTQANFLCLNCGYKENADYNASQNIAIKDIDKINIEETKGGES |
| 8 | MNKVVKLSLICEQTDKDGNKIEHGEVYKILWELQRQTREIKNKTIQYCWEYSNFSSDYYKINHEY |
| | PNEKEILSFTLKGFVNDKFKKGNDLYSGNCSTTTGNVCSEFKNSKSDFLKGEKSIINYKAYQPLD |
| | IHNKCILIEHTNNEFYVRLKLLNRPAIKKYNFANSEFNFKIIVKDNSTRTILERCIDKIYDVAAS |
| | KLIYDKKKKMWVLNLVYAFDNKSEYVLDKNRILGVDLGIHYPICASVYGEWNRFTIDGGEIEKFR |
| | KTVEARKKSMLRQGKNCGDGRIGHGISARNKPVYKIEDKIAKFRDTANHKYSHALIQYAIKNNCG |
| | VIQMEDLTGITNEADRFLKNWSYYDLQTKIKYKAKEVGIDIVYIKPKYTSQRCSKCGYIDKENRN |
| | KQASFVCLKCGFKANADYNASQNISIKDIDKLIEEMYNSSANTE |
| 9 | MSKGSLAKVMKYELRYLDGAGSFEQMQERLWVLQRQTREILNRSTQISFHWDYTSREHFEQTGQY |
| | LDVFSETGYKRLDGYIYSRVKDSCGDMASGNINATLQKAWNKYGTSKLDVLRGQMSLPSYKKDQP |
| | LVIEKHNIRLSMDGQQALAEITLFSNKFKKENSLSSNVRFAFQLHDGTQRRILNSVLSGEYGLGQ |
| | CQLVYDRPKWFLLLTYTFTPQNRQLDPDRILGVDLGECYALCASVFGEYGSLRIEGGEVTAYAKK |
| | LEARKRSLQKQAAVCGEGRKGHGTKTRVADAYQMQDRIANFRDTVNHRYSKALIDYALKNQCGTI |
| | QMEDLSGIRQDTGFPKFLQHWTYYDLQSKIENKAKEHDIRIVKINPRYTSQRCSKCGAIDSGSRT |
| | SQARFCCTKCGFTANADYNASQNISIKGIDLLIEKELGAKAE |
| 10 | MGKGEISKVMKYELRYLDGSGSFEEMQQRVWALQRKTREIQNRTVQIAFHWDYINREHFIQTGNN |
| | LNVLQETGYKRLDGYIYDRLKGQSAEMSGANLNATIQTAWKKYNSAKPKVLSGTMSVPSFKRDQP |
| | LIINSNCVKFSRSESECLAELTLFSREYKKEHDLSSNVRFAIRLHDSTQRSILERVLSGAYRKGQ |
| | CQLVYQRPKWFLFLTYSFFPMQHDLDPEKYLGVDLGECCALYASSVGEYGSLKLEGGEITAFAKQ |
| | LEARKRSMQKQAAYCGEGRIGHGTKTRVADVYKMENRIANFRDTVNHRYSKALIDYAVKHQYGTI |
| | QMEDLSGIKNDTGFPKFLRHWTYFDLQEKIDAKAREHGIHVVKVNPQYTSQRCSKCGSIDSRNRK |
| | SQKEFCCLNCGYKVNADFNASQNLSIKGIDVIIQKYIGAKSKQTENNG |
| 11 | MKEIAKVVKLELGWVFTDDGERAFPYSDLFEIQRQVALVKNKTIQLCWEWNNFGADFHAMSGAYP |
| | KTGDVTGYKTLDGYVYQRLKDDFSRMFKKNLNASVRSAQKAFETAKKSLHKGERSILSYRKDAPI |
| | ELHNSAVSFSQEGRKYKAAVKVFSLSYAKEKGYAGTGVEFELSHLQGSPKEIVQRCMSGEYKIGE |
| | SKLIWNEKKKKWFLYLTYKFTPAAVALDPEKIMGVDLGIACVAYMGFRFCEDRHVIPGHEVEHFR |
| | RRVEARKVELQRQGKFCGEGRIGHGRATRTKPVDQIGHAIARFRDTANHKYSRFIVDMAVKHGCG |
| | VIQMEDLHVHAEDKLLKDWTYFDLRTKLEYKAKEKGIEVRFVNPRYTSQRCSCCGYIEKENRKTQ |
| | KEFICLECGFAANADYNAALNLATAGIEQIIDEYVSANHK |
| 12 | MSKGTLAKVMKYELRYLDGFPSFYEMQEAVWGLQRQTREILNKTLQMAFHWDYTSREHFKETGSY |
| | LDVRTETGYKREDGYVYECLKSDYSDIASKNLNATLQKAWKKYRNTRLDVLKGTMSLPSYRSDQP |
| | LTLDKNTVKLHSDGVDDWVELTLFSKAYKTQHGLSANVRFAIPMHDRTQRSIFQNLIDGVYALGE |
| | CQLVYDKKKWFLLVTYVFTPEQHTLDPEKILGVDMGEAYAIYASSVYGYGTLKIEGGEVTDYAKK |
| | LERRKWSYQKQARYCGDGRIGHGTKTRIAEVYKAEDRIANYRDTINHRYSKAVVDYAVKNGYGTI |
| | QMEDLSGIKEDTGFPRRLQHWTYYDLQTKIENKAKEHGIRVVKIDPRCTSQRCSRCGHIDPKNRP |
| | SQSQFCCTACDFRANADFNASQNISTKGIDKIIAKTLRAKPE |
| 13 | MGTVTKVMKYELRYLDGSGSFHDMQNYVWQLQRQTREILNRTIQEATLWDYRSREHFLEAEEYLD |
| | VYAETGYKTLDGYIYNRLKGSYGDFAGANLNATLRKAWKKYKTSKTEVLRGTMSLPSYKGDQPLV |
| | LHNGSVKLQGDSRDAVVELTLFSNTFKKREGIKGNPSFSLLVRDNTQRSIYQSLIDGVYKLGECQ |
| | LVYQKKKWFLLLTYTFEARQHEVDPEKILGVDLGEAYAIYASSKDNFGSLKIEGGEVTDYAKGLE |
| | RRKRALQQQARYCGEGRVGHGTKTRVTEAYKAEDRLANFRKTINHRYSKALIDYAVKNGYGTIQM |
| | EDLSGIKADTGFPKRLQHWTYYDLQSKIEAKAKEYGIHVVKVDPSYTSQRCSKCGHIDSQNRKTQ |
| | ERFLCVSCGFSCNADFNASQNLSIKGIEKIIKKTKGAKVE |
| 14 | MAKKGNSQKKQIVKVMKYELKYEKGCADFNEMQNELWKLQRQTREVMNRTIQLCYHWSYVQAEYC |
| | KQHGCARRDVKPCDVYETNATSLDGYIYQLLKVEYPDFFMKNLNATLRKAHQKYDALLEDIQEGN |
| | SSIPSFKKDQPLIFEKKAICISKCLPDKRQITLSCFSDSYIDAHPTLDKITFTVRARSASEKSIF |
| | DHIISGKYALGTSQLVYEKKKWFFLLSYKFTPESVDVNPEKVLGVDLGVVNALCAGSVENPHDSL |
| | FIKGTEAIEQIRRLEARKRDLQKQARYPGDGRIGHGTKTRVSPVYQTRDAIARMQDTLNHRWSRA |
| | LIDFACKKGYGTIQMEDLSGIKAMESEKPYLKHWTYFDLQSKIIYKAEEKGIRVVKVNPKCTSRR |
| | CSACGYISKENRKNQAEFLCVNCGYHHNADYNAAQNLSIPQIDRLIEKQLKEQESEESEAGTNPK |
| 15 | MDLRNWLRRVRIHMAKGTVTKVMKYELRYLSGFSDFHAMQQAVWGLQRQSREILNKTIQMAFHWD |
| | YISRENFNANGVYLDVKAETGYKTYDGYIYNSLKSAYADMAAANLNAAIQKAWKKYKDAKMEVLR |
| | GTMSTPSYRSDQPVLINKNCVKLFDGGVRLTLFSDRFKRENNLNGNLEFAVQLHDGTQRSIFANL |
| | LNGTYALGQCQLVYDKRKWFLLVTYIFTPEKHELDPEKILGVDLGQTYALYASSVCARGTFRIEG |
| | GEAAECAHRLEQRKRSLQQQARFCGEGRVGHGTKTRVAAVYSAGDKIASYRDSINHRYSKALVEY |
| | AVKNGYGTIQMEDLTGIQNDLDHPKRLQHWTYYDLQTKIENKAKEHGVGVVKVNPRYTSQRCSRC |
| | GHIERENRPTQKVFCCKACGFEGNADYNASQNLSMRNIDKIIEKELSAKGE |
| 16 | MSGSTIAKVMKFELFYREGGGEFHEMQKLLWELQRQTREVLNKSVQIYYQWKWKKQQHFEETGQS |
| | LDIYTELHYHRISSYAYNVLKEKYSSFYKANLSSTIKTACDKCESSEKDILCGTMSVPSYKRDQP |
| | LLLHDTSLSIRRNGTQWFADCKLFSAELVKNLGLKRGQSLVFSIKALDKTQINILERIEDGNYAI |
| | RQSQLTYEKKKWFLYLTYRFDKPKSELDPNRILGVDLGVSNAFCASVYGELDKLMIPGDEAIETI |
| | RRLEQIKYSKLRQARYCGEGRIGHGTSTRIAPAYSTRDKISNLQKTLNHRWSHAIVRYALRQGCG |
| | VIQMEDLSGIKEANDFPLRLQHWTYYDLQTKIKNKANEYGIEVRQVDPQFTSQRCSKCGCIQKEN |
| | RPAQAKFCCIKCGYRTNADYNASQNLALPDIDRIIQEELKQIGANRK |
| 17 | MQQVVRFEILKPVDNDWKILGRVFRELQYESRLVLNKTIQYNWEFSNCVIGFKEKFGIAPKISDI |
| | SKYKDGKRGLEGYIYDKLKDIYTKNYSKNLGCLISKATSQWYAIKNDVYKGEKIPPEYKKSNTPI |
| | IVDKQAMTLFKENNLYYAKVALVSTNHIKEYGLNSCKFTILLNTKNNGNKVILDKVINGEFDYSQ |
| | SQIEKNKKWFLYLSYKIKEKTLPDDYNRVMGIDLGKNKAVVIAVHGTEIRDYILGGEIIDYKKKM |
| | YNMVWHRQKQSRYCGEGRIGHGRKTRLKDVYNIKEKIANFSDLTNHKYSKYIVELAKRHKCGIIQ |
| | MEDLSGLSTDNKFLKQYPIYDLQQKIIYKAEREGIKVVKIKPHFTSQMCSNCHYISTQNRPKDDR |
| | GWEYFKCVNCGLEIDADLNAARNIANPQIELIIEEQLKIQEIDDKI |
| 18 | MAEKTIVKVMKFELRYIDGAGEFSEMQKHLWELQKQTREVLNKTIQMGYALECKRFAHHDKTGQW |
| | LDDKELTGSKYKAVADYINAELKEDYNIFYSDCRNSTVRKAYKKFKDAKNKIFSGEMSLPSYRSN |
| | QPIIHNRNVIIRGNAESALVGLKVFSDGFKALHGFPAAVNFKLCVKDGTQRAIIENVISEIYKIS |
| | ESQLIYDNKKWFLILAYRFTQKKNDLNPDKILGVDLGVKFAVYASSIGEYGSFRIKGGEVTEFIK |
| | RLEKRKKSLQNQATVCGDGRIGHGTKTRVADVYKARDKISNFQDTINHRYSRAIVDYARKNGYGT |
| | IQLEKLDNSIEKKGDYSPVLVHWTYYDLRTKMEYKAAEYGIKVIAVEPKYTSQRCSKCGYISSEN |
| | RKTQESFECIKCGYKCNADFNASQNLSVRDIDRIIDEYLGANPELT |
| 19 | MANEFTCITRKIEVHLHKHGDSDEAIQRYKEEYRMWDDINNNLYKAANRIVSHCFENDTYEYRLK |
| | LHSPRFQEIEKLLSNPKRNKLSDDDIKELKAERKLLFSDFKSQRQTFLRGGIETGTNPEQNSTYK |
| | VISNEFIDCIPSEVLINLNQNISSTYREYTLDVERGIRTIPNFKKGIPVPFSIKQHGEIALKKRD |
| | DGTIYVRFPKGLEWDLNFGRDRSNNREIVERVLSGQYGVGNSSIQESKNKKQFLLLVVKIPKENR |
| | VLDKERIVGVDLGVNTPLYAALNDNEYGGMGIGSREQFLKVRERMNAQKRELQRNLRHSTNGGHG |
| | RSQKLQALDRLEGKERNWVHLQNHIFSKSIIEYALKNDAGVIQMERLTGFGRDNNEEVQNEYKYI |
| | LRYWSYFELQTMIEYKAKAAGIEVRYINPYHTSQTCSFCGHYEKGQRINQPTFICKNPDCTKGKG |
| | KQKSNGAYEGINADWNAARNIARSNEFVEKKKK |
| 20 | MATEYTCITRKIEVHLHRHGDSEEDTQRLKDEYHIWDVINDNLYKAANRIVSHCFFNDAYEYRLK |
| | LHSPRFQEIEKLLRYSKRNKLTDEDIKQLKAERKELFSIFKKQRLEFLQGGSGKGSEQNSTYKVV |
| | SNEFGEIIPSHVLTCLNQNITSTYSAYSKEVEYGNRTIPNFKRGIPVPFPIKQQGTLQLKRREDG |
| | SIYIRFPLGLEWDLSFGRDRSNNREIVERVLNGQYDVGNSSIQETKNKKRFLLLIVKIPKQAVTL |
| | NPDRIVGVDLGINIPLYAALNDNEYGGMGIGSREQFLKMRMRMAAQKRELQRNLRHTTHGGHGRT |
| | QKLQALERLEGKERNWVHLQNHIFSKSIEYAQRNDAGVIQMERLTGFGRDKHDEIDSDFKFILRY |
| | WSFFELQTMIEYKAKAAGIEVRYIDPYHTSQTCSFCGHYEKGQRISQSTFVCKNPDCEKGKGKKH |
| | SDGTYEGINADWNAARNIALSTKIVDRKKK |
| 21 | MATEYTCITRKIEVPLHRHGEDEEAKQRLIDDYRVWDTINDNLYKAANRIVSHCFFNDAYEYRLK |
| | IHSLRFQEIEKLLKYSKRNKLTDEDIKQLKAERKQLFADFKKQRHTFLRGGVAEGANPEQNSTYK |
| | VISNEFLEVIPSEILTNLNQNISSTYKNYSLDVERGIRTIPNYKRGIPVPFSIKQRGELMLKRRD |
| | DGSIFIRFPMGLEWDLSFGRDRSNNREIVERVLSGQYDVGNSSIQESKNRKRFLLLVVKIPKENH |
| | NLNPDRIVGVDLGINIPLYAALNDNEYGGMGIGSREQFLNMRMRMDAKKRELQRNLRQSTNGGHG |
| | RKQKLQALERLEGKERNWVHLQNHIFSKSIIEYAVKNNAGAIQMERLTGFGRDKNDEVDSDFKFI |
| | LRYWSFFELQTMIEYKANAAGIEVRYIDPYHTSQTCSFCGHYEKGQRLNQSTFVCKNPDCEKGKG |
| | KKLSNGTYQGINADWNAARNIALSDKIVDRKKK |
| 22 | MATEYTCITRKIEVHLHKHGDSEEAAQRFKEEYRIWDDINNNLYKAANRIISHCFENDTYEYRLK |
| | LHSPRLQEIEKLLSNPKRNKLSDEEVKQLKAERKQLFADFKKQRHVFLRGGVEEGANPEQNSTYK |
| | VVSNEFIDFIPSEVLTNLNQNISSTYREYSLDVERGVRTIPNYKKGIPVPFSIKQKGEIVLKKRE |
| | DGSMYVRFPKGLEWDLNFGRDRSNNREIVERVLSGQYDVGNSSIQETKNKKRFLLLVVKIPKQVA |
| | SFDPSRIVGVDLGINVPLYVAINDNEYGGMGIGSREQFLKMRMRMAAQKRELQRNLRHTTNGGHG |
| | RTQKLQALNRLEGKERNWVHLQNHIFSKSIEYAVRNNAGVIQMERLTGFGRDKNDEVGADFKFLL |
| | RYWSFFELQSMIEYKAKATGIEVRYINPYHTSQTCSFCGHYEKGQRINQATFVCKNPECTKGKGK |
| | QRTDGTFEGINADWNAARNISFSTDFVDKKKK |
| 23 | MATEYTCITRKIEVHLHRHGDSEEAAQRLKEEYRIWDEINDNLYKAANRIISHCFFNDAYEYRLK |
| | LHSPRFKEIERLLKYAKRNKLTDDDIKALKAERKELFAEFKRQRQSFLGGSEQNSTDRVVSHEFL |
| | DVIPSEVLTCLNQNIASTYKEYARDVERGVRTISNFKKGIPVPVRVKRNGALLLRKREDGSIYLS |
| | FPKGLEWDLNFGRDRSNNREIVERVLSGQYDVGGSSIQEAKNGKRFLLLVVKIPKESRALNPDRV |
| | VGVDLGVNIPLYAALNDNTYGGLSIGSRDQFLKVRMRMAAQKRELQRNLRVATNGGHGRKQKLQA |
| | LDRLEGKERNWVHLQNHIFSKSIEYALRNEAGAIQMERLTGFGHDRNDEVDEGFKFILRYWSFFE |
| | LQTMIEYKAKAAGIEVRYVDPYHTSQTCSFCGHYEKGQRVNQATFICKNPDCTKGKGKERSDGTF |
| | EGINADWNAARNIALSDKIVERKKK |
| 24 | MATEYTCITRKIEVHLHKHGDSEEATQRLKNEYHIWDEINNNLYKAANRIVSHCFENDTYEYRLK |
| | LHSPRFQEIEKLLNNSKRNNLSAEEIRQIKIERKLLLSEFKKQRYAFLRGGIEEGANPEQNSTYK |
| | VVSNEYIDKIPSDVLTNLNQNISSTYKEFSLDVEKGVRTIPNYKKGLPIPFSIKKNGDLLLKKRD |
| | DGTIYIRFPKGLEWDLSFGRDRSNNREIVERILSGQYDVGNSTIQETGNKKRFLLLVVKVPKKNI |
| | VSNPNRVVGVDLGINYPLYAALNDNEHGGISIGSRDQFLKMRMRMAAQKRELQRNLRHTINGGHG |
| | RTQKLQALERLEGKERNWVHLQNHIFSKSIIEYAIKNNAGTIQMERLTGFGRNENNEVGSEYKFL |
| | LRYWSFFELQTMIEYKAKASGIDVRYINPYHTSQTCSFCGHYEKGQRLNQATFVCKNSACTKGKG |
| | KQKSDGTYEGINADWNAARNIALSTDFVDKKKK |
| 25 | MATEYTCITRKIEVHLHRHGDSEEAAQRLKEEFRIWDEINDNLYKAANRIISHCFFNDAYEYRLK |
| | LHSPRFQEIEKLLKYAKRNKLTDDDIKALKAERKELFAEFKRQRQSFLGGSEQNSTYKVVTDEFL |
| | EVIPSHVLTCLNQNISSTYREYALDVEHGRRTIPNFKKGIPVPFPIKATGELLLRKREDGSIYIR |
| | FPKGLEWDLNFGRDRSNNREIVERVLSGQYDVGNSSIQETKNRKRFLLLVVKIPKESRALNPDRV |
| | VGVDLGVNIPLYAALNDNTYGGMSIGSRDQFLKVRMRMAAQKRELQRNLRVATNGGHGRKQKLQA |
| | LDRLEGKERRWVHLQNHIFSKSIEYALRNEAGAIQMERLTGFGHDRNDEVDEGFKFILRYWSFFE |
| | LQTMIEYKAKAAGIEVRYVDPYHTSQTCSFCGHYEKGQRVNQATFICKNPDCTKGKGKERSDGTF |
| | EGINADWNAARNIALSDKIVERKKK |
| 26 | MATDVTCITRKIEVHLHKHGDSEEGAQRLKEEYRIWDDINNNLYKAANRIISHCFENDTYEYRLK |
| | LHSPRFQEIEKLLSKPKCNKLSADDIKQLKAERKVLFADFKKQRQVFLRGGLEEGTNREQTSTYR |
| | VASKEFIDTIPSEVLTNLNQSISSTYKKYALEVERGVRTIPNYKKGIPVPFAIKHKEELALKKRD |
| | DGSIYVRFPKGLEWDLSFGRDRSNNREIVERVLSGQYDVGNSSIQETKNKKRFLLLVVKIPKENR |
| | VLNKERVVGVDLGINTPLYAALNDNKYGGLSIGSRDQFLKVRMRMTAQKRELQRNLRHTTNGGHG |
| | RTQKLQALDRLEGKERNWVHLQNHIFSKSIEYALQNDAGVIQMERLTGFGHDNNDEVDEKFKFIL |
| | RYWSFFELQTMIEYKAKAAGIEVRYINPYHTSQTCNFCGHYEKGQRINQATFVCKNPDCIKGKGK |
| | QHSDGSFAGINADWNAARNIALSNDVVDKKKK |
| 27 | MTAEYTCITRKIEVHLHKHGESEEATQRFKDEYRIWDDINNNLYKAANRIISHCFFNDAYEYRLK |
| | LQSPRFQEIEKLLGNTKRNKLSAEDIKVLKAERKLLFSDFKKQRQIFLRGGVEEGPNPEQNSTYK |
| | VVSQEFIDVIPSEVLTNLNQNISSIYREYALDVERGIRAIPNYKKGIPVPFSIKQKGEIVLKKRE |
| | DGSIYVRFPKGLEWDLNFGRDRSNNREIVERVLNGQYDAGNSSIQETKNKKRFLLLVVKIPKESR |
| | SLNKERIVGVDLGINVPLCAALNDNEYGGISIGSRDQFLKVRMRMAAQKRELQHNLRHTTTGGHG |
| | RTQKLQALDRLEGRERNWVHLQNHIFSKTIIEYALKNNAGVIQMERLTGFGRDGKEEVQNEYKFI |
| | LRYWSFFELQTMIEYKAKAVGIEVRYINPYHTSQTCSFCGHYEKEQRISQTTFVCKNPKCTKGKG |
| | KLKSDGTFEGINADWNASRNIAKSTEFVDKKKK |
| 28 | MATEYTCITRKIEVHLHKHGDSEEATQRFKDEFRIWDDINNNLYKAANRIITHCFENDAYEYRLK |
| | LHSPRLQEIEKLLSNSKRNKLSDEEVKQLKAERKQLFADFKKQRQVFLRGGVEAGANPEQNSTYK |
| | VVSNEFIDTIPSEVLTNLNQNISSTYREYSLDVERGIRTIPNYKKGIPVPFSIKQKGEIVLKKRE |
| | DGSIYVRFPKGLEWDLSFGRDRSNNREIVERVLSGQYDVGNSSIQETNNKKFLLLVVKIPKQMAS |
| | VDPNRIVGVDLGINVPLYAALNDNEYGGMGIGSRDQFLKVRMRMAAQRRELQRNLRYTTNGGHGR |
| | TQKLQALDRLEGKERNWVHLQNHIFSKSIEYAVRNNAGIIQMERLTGFGRDENDEVGTDFKFLLR |
| | YWSFFELQSMIEYKAKAANIDVRYINPYHTSQTCSFCGHYEKGQRINQSTFVCKNPECAKGKGKQ |
| | RADGTFEGINADWNAARNIAFSTEVVDKKKK |
| 29 | MATEYTCITRKIEVHLHRHGDSDEAIQRYKDEFHIWDEINNNLYKVANRIISHCFFNDTYDYRLK |
| | LHSPRFQEIEKLLRNPKRNKLSGEDVKRLKAERKALDADFKKQRQAFLRGGVEEGTNKEQTSTYI |
| | VVSHEFIDIIPSEILTNLNKNIFSTYKKYRLDVEKGARTIPNYKKGIPVPISIKRSGELMLKKRE |
| | DGSIYVRFPKGLEWDLFFGRDRSNNREIVERVLNGQYDVGISTIQETKNKKRFLLLVVKIPKESK |
| | NLNPNRVVGVDLGINIPLYAALNDNEYGGLGIGSREQFLKVRMRMAAQKRELQRNLRHTINGGHG |
| | RAQKLQALDRLEGKERNWVHLQNHIFSKSIIEYALRNGAGVIQMERLAGFGRDKNEEVENEFKFI |
| | LRYWSFFELQTMIEYKANAAGIEVRYIDPYHTSQTCSFCGHYEKGQRINQSTFVCKNPDCVKGKG |
| | KQHADGSYDGINADWNAARNIALSTTVVDKKKK |
| 30 | MATEYITKTRKIEVYLHRHGDSDEAKQRYQQEWQIWHDINDNLYKVANRIMTHHFLNDEFVSRLR |
| | STNPRYVEIEKILKHCKRNKLSQEEINSLQQENRALDALFTEKKNEFLGTTQEHNTILRIVRKEF |
| | GDVIPNDVYDCVIAERVKYTDKQKHLQIINGESSVPNYRKGMPVPFRIKIGANHNTLGILRRNDN |
| | PNHIYVKFPKGLEWDLVFGKDPSNNRKIVERILSGQYDAGNSSIQQAKNGKCFLLLVVKIPKSNI |
| | QLNKDRVVGVDLGINIPLYAALNDNIHSRLSIGSREQFLKMRMRMYAQKRELQRNLRHSTNGGHG |
| | RKQKLQALERLEGKERNWVHLQNHIFSKSVIEFAQKHNAGVIQMERLTGYGKDANGEMREEAKFL |
| | TRYWSYFELQTMIEYKANAAGIEIRYIDPYHTSQTCSFCGHYEKGQRVSQSTFICQNPECKQGKG |
| | KQKSDGTFEGINADWNAARNIALSTQYVDKKKK |
| 31 | MATEYTCITRKIEVHLHRHGEDEDAVQRYKNEFQIWNEINNNLYKVANFISSHLFFNDAFVDRLR |
| | VQSNEYRDLLDLISKTTDAKEIKALENRKKALDAEFKRQQKIFLKGGSEDEKGSEKTAIRRIAVE |
| | TFPNIPYSIINSLNDQISKTYNSSRFDVSIGKRTVPNYKKGIPVPFLMANGSGKIALREREDGSP |
| | YVLFPRGLEWDLHFGKDSSNNREIVKRVFNGEYKACDSSLQQAKNKKIFLSLVVKIPKKNHNLNP |
| | DRIVGVDLGINIPLYAALNDNDYGGMGIGSREQFLKVRMRMSAQKRELQRNLRQSTNGGHGRAQK |
| | LQALERLEGKERNWVHLQNHIFSKSIEYALKNNAGAIQMERLTGFGRDKNDEVDSNFKFILRYWS |
| | FYELQTMIEYKANAAGIEVRYVDPYHTSQTCSFCGHYEKGQRLNQSTFVCKNPDCEKGKGKKLSD |
| | GTYQGINADWNAARNIALSDKIVDRKKK |
| 32 | MNDSPVIKNKRHVKVLRLRILKPVSGTWQDLAKLLRDTRYRVYRLANLAVSEAYLGFHMWRTGRA |
| | ETYELDTPGALNRRLRRMLDEEGVRADELDRFSKTGALPDTVVGALSQYKIRAATGKSKWQEVIR |
| | GKSSLPTYRLDMAIPLRCDKRNHARLARVENGDVTLDLMLCLRPYPRVVIQTGNIGGGAQAVLDR |
| | LLANPSQNPDGYRQRLFEIKHDDRDNKWWLYITYDFPAADPPRSSADRIVGVDIGVSCPIYVAIN |
| | DGHARLGRRQFSSLGARIRSLQNQIVARRRSMQAGGKVALSGQTSRSGHGRKRKLRPIQKLEGRI |
| | SHAYTTLNHQLSSSVIDFALSHGARVIQMEDLASLKDALRGTFIGARWRYHQLQQFLEYKAKESG |
| | LTLRKINPQFTSRRCSRCGFIHVEFDRARRDASRRDGYVARFVCPAPKCGFEADPDYNAARNIAT |
| | PDIEKLISDQCKIQSIPTRSLTDQSEAADKDTLAQGQSRSGG |
| 33 | MSEKRHNKVAKFQILKPAAGTTWPELANLLFAVRYRVFRLANLCISEHYLHYHLWRMGKTEEIPK |
| | LKISELNKKLREMIIEENDKKEKQNKINQDAINKKGALTSYVVDTLSQNKLGAVTSKSKWKEVIL |
| | GKASLPTFRLNMAIPVRCDKPEQCRLKINANGDVELELMICERPRPRIILKTGGLSGSMKSVLDR |
| | LLENSAQSMEGYRQRNYEIIQDRNDGKKWYLHVSYDFPATERKPNSEIIVGVDVGFALPLYAALG |
| | NGHARLGWKQFHSLAKRIRSLQNQVVSRRRKMLRGGKDSLTQDTARSGHGRNRLLQPIEKLAGRI |
| | EKAYTTLNHQLSRSVVDFAKNHGAGIIQMEDLEGMKDAINGTFLGERWRYFELRQFIEYKAKEAG |
| | IEVRLANPKYTSRRCSACGYINMAFTREYRDSHRKNGKSAEFFCPECDKLPADDEQPQKPYPTDA |
| | DYNAAKNLAALDIEKIIRRQCEKQGISYDKSPENNDL |
| 34 | MANTEFTCITRKIEVHLHRHGDSDEAALRLKNEYHFWDEINDNLYKAANRIISHCFFENDTYEYR |
| | LKLHSPRFQEIEKLLKNAKRNKLTDEEIKELKAERKLLFSDFKKQRYTFLRGGIEDGANPEQNST |
| | YRVVSNEFIDTIPSEVLTNLNQNISSTYKNYTIDVERGLRTIPNYKRGMPVPFSIKKHGVLALRK |
| | REDGSIYVAFPKGLEWDLSFGRDRSNNREIVERVLSGTYDVGNSSIQEAKNGKRFLLLVVKIPKE |
| | VKILDTSRVVGVDLGVAVPLYAAISDNEYGGMSIGSYDQFIKVRMRMNAQKREMQHNLRHTTNGG |
| | HGRKQKLHALERLEGKERNWVHLQNHIFSKSITEYALQNNAGAIQMERLTGFGHDKNNEVDEGYK |
| | FILRYWSFFELQTMIEYKAKAAGIEVRYIDPYHTSQTCSFCGHYEKGQRLNQSKFVCKNPDCVKG |
| | KGKQRSDGSFEGINADWNAARNIALSTKIVDRKKK |
| 35 | MENNITITRKYALIPEFSDRKEWKKRVYDFMINDLEQKIDYRNKKKQDTSELESQLEYIKNGGDF |
| | TRSMVNNYTYSLVRKAMEEETRRKNYILSWIFSEMRANRIDQMESLKDKFKFVSDTINYAYRKAG |
| | SNKGSLFDETEIHCILKSYGIAFSQELTKEIKELVKNGVLEGKVVIPTYKLDSPFTIAKSHFSFE |
| | HDYDSFEELCEHINDSDCKMYMNYGGDNRKDGINPASIARFRISLGHGKNKDELKSTLLKVYSGE |
| | YQYCGSSIQITKNKIILNLTMKIPKIETKLDENTVVGVDLGIAVPAMCALNNNMYERLAIGSADD |
| | FLRTRTKLQSQRRRLQKSLKNSNGGHGRNKKLKVLERLGKSETHFAETYCHMVSKRIVDFALKNN |
| | AKYINIENLNGYNTSSFILRNWSFYKLQQYITYKAERYGIVVRKINPCYTSQVCSVCGNWEDGQR |
| | KTQASFECANPKCESHKKYKYGFNADFNAARNIAMSTLFMEDGEVTEKKKEEAREYYGIKKENSE |
| | AV |
| 36 | MATEYTCITRKIEVHLHKHGDGEEAEKRRAEEFRMWNEINDNLYKAANRIVSHCFFNDAYEYRLK |
| | IQSPRYKEIQRKLRYSKSNKLTDDEIKSLKAERKELDNEFRKQYRAFMLGGSSEGFKSTTEQNST |
| | ERIVNNEFGDIIPSNVLSCLNQNVFQTYKQYRTDVEFGKRTISNFKKGMPVPFSIKAHKSLMLKK |
| | REDGSIFVYFPKGLEWDLSFGRDRSNNREIVERILSGQYDAGTSSLQEGKNGKIFLLLVVKIPKQ |
| | SNALDPNRVVGVDLGINIPLYAALNDNEYGGMSIGSREQFLKMRMRMVAQKRELQRNLRHSTNGG |
| | RGRSHKLQALERLEGKERNWVHLQNHIFSKNIIEFAVKNNAGVIQMERLTGFGHDRNDEVDDGFK |
| | FILRYWSFFELQSMIEYKAEAAGIEVRYIDPYHTSQTCSFCGHYEKGQRIDQATFVCKNPECEKG |
| | KGKKRSDGTYTGINADWNAARNIALSDKFVDKKKK |
| 37 | MANDEICITRKIEVHLHLHGDDEEGRARRKKDFETWNTINDNLFKVANLIATHQFFNDAYEFRAK |
| | IHSPQYTKIEKDLNNSQKLKLTQEQVRELEKEKSKLDKEIEEQRKTFLQCSRQNSTFRVASKLFL |
| | DVIPSNVLTCLNQKICSTYTSYKSEVESGKRTLPNFKKGLPVPFQMNLNKKLQLRRRTDGSIFVL |
| | FPKGLEWDLFFGKDKSNNREIVNRVLSGEYGVGESSIQQNKKGKTFLLIVVKIPKKAICLDSKRV |
| | VGVDLGINVPLCAALNDNESAKMYIGSREQFMKVREQLYVRKRELQRSLTTSTRGGRGRKQKLQA |
| | LERHEGKERNWTHLQNHIFSKSVIEFAQKQNAGVIQMENLSGFGRDKNDEVDEGYKYILRYWSYF |
| | ELQQMIEYKAKASNIEVRYVDPKYTSQTCSYCGHYEKGQRISQSTFVCKNPECEKGKGKKTKDGK |
| | YEGINADWNAARNIALSGKVEERKKKKKRKIQSNQ |
| 38 | MNTINNTYIRTLKFNLNLTPKFETEEDNKKYINDVYSYLRDAMWAQNRAMNIVLDRTKEAYTLGR |
| | GMNRVKEIYYSYSHQKPISNDKKESFLESLLQYAPIDDQFVKNEVKKLRKFYESKKKPPKEETVS |
| | KNCEALKNKYIKYVGKSKDDIKRELDLLENYCAYPEDIYEKFANGLSTPAYIKQKVESYWKQDGI |
| | KTKVIYSMDENLRRIKDAPLFIPPNIFYNKKDELIGLVYDYTDYISFLEDLENKRNVNIYLSIPY |
| | KKGEDKLKFKLVLGNPHKSRDSRLSIKRIFEEEYRIKGSSIGFTKNKETGKNTNLTLYLTVEVPQ |
| | NKDNTLDENVVVGVDVGIAIPCVCALNNDKYTRENIGSYDTLFAKRTQFKMQRSRLNSQLKLSKG |
| | GHGRKRKLKKLELLSGKEKNYADTECRKYASDVIKFCLKNHAKYINLEHLKGYRENPKVLAGWSF |
| | YKIQTYIEQAAEKHGIIVRKINPCYTSQICSVCGNWHPENRPKGKLGQAYFNCHNIDCKTHNTDL |
| | YKYGINADFNAARNIAMSTLFITDSDEITKKHWKEAREYYGIDESDDKEEKLNKVA |
| 39 | MIIARKLKITVIGSDEERKEKYRWIRDEQYNQYRGLNMGMTYLATGEILRMNESGLEIRLEKQKT |
| | ELESKVEKAKLNIEKIKIKIEKIKTSKKINEEKILENQNKIDDEKANIIKYKNNIIKIEQALKVA |
| | KIKRMDIQMEFKEKYIDDLYQVLDKVPFQHLDNKSLITQRVKNDIKADKTSGLLKGERSIRNYKR |
| | TFPLLTRGRDLKFYYDDKDIKIKWIEGIEFKVVLGNRIKNSLELRHTLNKVVNEEYKICDSSLQF |
| | DKNNNLILNLTLDIPENNKNEKVEGRVVGVDLGMKIPAYVVLNDVEYIKKSIGSIDDFLKVRTQM |
| | QSRRRKFQKQLQSANGGRGRNKKLQSLSRFEEKEKKFAKTYNHFISSNIIKFAVDNKASQINLEF |
| | LSLKETQEKSVLRNWSYYQLQQFIEYKAKREGIGIKYVDPYHTSQTCSVCGNYEEGQREIQEKFI |
| | CKNPKCKCELNADYNAARNIAKSTKYIKSKEESEFYKLNKKE |
| 40 | MTKVVKLALICEQNDKDNNPIDYKEIYKILFELQRQTREIKNKSIQYCWEYSNFSSDYYKLNHEY |
| | PKEKEILSYTLDGFVNDKFKNGNDLYSGNCSTTVRSACGEFKNSRSDFLKGTKSVINYKGNQPLD |
| | LHNKAIREDCIGKEYYVYLKLLNRPAFKKHNYANTEIRFKVLLYDNSSKTIVERCIDKIYKISAS |
| | KLIYNEKKKCWMLNLSYSFSNDSETELDKDKILGVDLGIHYPICASINGERKFFKIDGGEIEHTR |
| | RKIEARKKSLLKQGISCGEGRIGHGIKTRNKPVYDIEDKISRFRDTANHKYSRALINYAVNNNCG |
| | IIQMEDLTGITSDSNRFLKNWSYFDLQTKIEYKAQEVGIKVVYIDPHYTSQRCSKCGYISKDNRT |
| | EQALFWCQKCGYKTNADYNASQNIAIKDIDKIINAKK |
| 41 | MLKIKIECLNREVKNIITTRALKLTIVGDEETRNKQYKYIRNEQYEQYKALNLCMSLLNTHYVLN |
| | SYNTGAENKLKNQLEKLQNKIDKNNLELEKEDIKNSKKEKLTKQNMQFKGELIKLQEEYNKASKY |
| | RSDVDLAMKDMYIDDLYMAIQNQVTFKNKDFMSLVTQRAKKDFKNCLINGLARGERSLTNYKRNF |
| | PLMTRGERWLKFRYKEESDDILIDWIQGITFKVILGSRKNENTTELRHTLHKVITGEYKICDSEM |
| | KFDKSNNLMLNLIMDIPVKENTNYIDGRVLGVDLGIKYPAYVCLSDDTYKRMAIGSAQDFIRVRE |
| | QIRTRRFRLQEQLKMVKGGKGREKKLGALERIKDKERGFVKTYNHMVSKNIVEFAYKNKCEYIHV |
| | EDLNKNGFDNAILSKWSYYELKTMIEYKAERKGIKVRYVNPEYTSQKCSKCGHTDKENRQSQEKF |
| | KCLNCGFELNADHNASINIARSNDIKK |
| 42 | MADFVITRRIEVHLHHDPVADPEKVEYNRQWEYWRTINNNLYLAANRISSHLFFTDEYEHRLRIQ |
| | HPRYRDIERTLASVSKVKRMSKEEIAALRVERRTIEQELRAQTAAFLQTSRQNVTYRIASDEFGD |
| | LIPSDVLTNLNQNITSTYNEFKKQVVRGERTLSNYKKGIPVPFSMKKGEGLRRRDDGSYYVLFPG |
| | GLEWDLAFGRDRSNNRAIVERAFNGDYEVGNSSLQEKNRKVFLLLVVKIPAQELALDPNRIVGVD |
| | LGLNIPLYAALNDNEYGGLAIGSREQFMKVRERMSARRRELQRALRHSTQGGRGREHKLQALERF |
| | QAKERNWVHTQNHIFSRAVVEYAKQNSAGVIQMESLKSFGRDKEDHIEAGFRYVVRYWSYFELQT |
| | LIAEKAKREGIEVRMIDPYHTSQTCSFCGHYEKGQRISQGVFICKNPECAKGKGRQLKDGTYSGI |
| | NADWNAARNIALSTEVVKK |
| 43 | MITARKIQVTIVSNNRDEDYRFIRNEMREQNKALNVGMNHLYFNYIARQKLRLADLAYQEKESKL |
| | VSQIDKIYDDIKKAKTDEKREQLKEKLEKQKKKLEKMRKQKNNDLFQQYQQIIGTSEQTSVRDAI |
| | SEQFNLMSDTKDRLSQKVTQDFKNDIKAGLLTGERVLRTYKKDNPLYIRGRSLNLCKEEDTFYFK |
| | WIKGIVFQCVLGIKGQNKTELYKTLERVLDGNYKICDSALQFNKNNKLILILTLDIPDAVRESKI |
| | EGRVVGVDLGLKIPAYCSLNDDKYPRLAIGDIKDFLKVRTSLQRQYRSLQRALKSSKGGKGRYKK |
| | LKALDRFREKEKNYVTTYNHFLSREIVKFARKYRAEQINLELLSMAESTNKSVLRNWSYYQLQQF |
| | IEYKAAREGIKVKYVDPYRTSKTCSECGHFEEGQRTDQAHFTCKQCGFEANADYNAARNVAKSTK |
| | FITTKEQSEYYQKDSVS |
| 44 | MNKVIKIYASLDKAQQCALPIYGKDSLSVQLLKVQSEVRSLKNRAMRMSYDYDQAQYEYFKYMER |
| | CKQEYGLNSYPSAQPSDFSKYKTFDGYLYDALAKDYPLMNKRISATVTRKVWGEYKKDKGEILSG |
| | KKSLRTYREGQPIPIRAKDTKLLYEDNFDYTMTVSVFSKDAAKTLGMKPGGCRFILHEQTDSEKA |
| | ILDRLLSGEYKLCETLLAYDDRKNHDTKMARGWYFCIGYSFEKDTSNPSLDKDKILGVDIGVANV |
| | IYLGWSKDDHFKKYIPGSEIRKFQATEERRKKDILRCSVARGDGNVGHGRKCATRKAEKHEHHIH |
| | NFKETKNWHYAHFVVDTAVENGFGTIQMEDLSGINKSETEDRTWTFYSLQQKIEQLAAENGIVVK |
| | KVKPQYTSQMCSKCGYISSRSRRSQSEFRCVSCDYRRNADHNAAMNISKSKIEALVEEQLLRQGG |
| | EAD |
| 45 | MYVEFIRIKGGEILITARKIKLTIAENREEGYSFIRKELQEQNKALNMAINHLYFNYVAREKIKL |
| | ADETYKVKLEEGECYLERKYIELKEAKTDKQKENIKKSIEATKKKLETLRKVENKEVSNNFKEII |
| | ATSEQINLRDLISNNFNLKSDTKDKLTQKVVQDFYNDIVGVLRGERTLRRYKKDNPLYIRGRSLT |
| | LYREGEDYYIKWMNGIVFKCVLGVKKQNSLELQKTLDKVIFEEYKLCDSSIGFKDNKLILNLTLD |
| | IPVSNTNKFEKVIGRIVGVDLGMKIPAYCALNDSEDVRKAIGSIDDFLKVRTQMRSRRRKLQRAL |
| | KSTNGGQGKNKKLSALNSFEAKEKNFAKTYNHFLSSNIIKFATDNKAEQINMEFLSLSETQNKSV |
| | LSNWSYYQLQQMIEYKAERIGIKVKYVDPYLTSQTCSECGHYEDGQREVQSEFQCKKCGCKTNAD |
| | YNASRNIAKSDKYITKKEESEYYKNKI |
| 46 | MRDKLKASFAGEKLKKIKNAQISSPSATSDRFPIPMWQQTGFRVETNNEDLVIDIPFPLYKYREE |
| | VDKFKPWEKLEFIDTSKKKHIQLILSTNSRKRNVGWVKDWSTEAEIKRVMSKKLVINKIEITRGK |
| | RINERNYWFVNFVIAYEKPVRKLDSKITGGIDVGVSNPVVCAINSGLNRLTIRDNDVVDFTRAEL |
| | ARLRSQRRGDRFRRGGHGIKHKFKPSETIQKKYEQRRKKKMEEWASRITRFFLNNGVGLVYMENI |
| | DKKSISDGEDYFKVQLRVTWPVKEMQKLFERKLKENGIEVKSIDPKYSSQLCSKCGKWNTHENFR |
| | YRQLNGYPPFECKYCDYGKDKEKEIIYADYNAARNLANPNEDKRRKVAIPSEVLKGNIKEEVVAE |
| | N |
| 47 | MITVRKLKLTIVGDEETRNQQYKLIRDEQYQQYRALNLCMSLLSTYNILNNWNSGAENKLNSQIE |
| | KLNKKVEKNKNDLKKDNLKENRIKKINESIQTLTKEKEKLQQEYLSSSEYRSDIDKKVKEMYIDD |
| | LYTVVQSQVNFKSKDMMSLVTQRAKKDFTTALKNGMAKGERSLTNYKRDFPLMTRGERWLKFEYD |
| | EDSDDIYINWLHGIRFKVVLGYKKNENSIELRHTLHKVINKEYKICDSSMQFDRNNNLILNLTLD |
| | IPLNIKNEHIEGRTLGVDLGIKYPAYVCLSDDTYKRKSIGCAEDFIKFREQIRARRYRLQKQLSM |
| | VKGGKGRNKKLQALDRIKDKERNFVKTYNHMISKNVVEFAKNHKCESINLEKLTKYGFPNMILSK |
| | WSYYELQNMIEYKAEREGISVKYVDPAYTSQTCSKCGHVDKENRTSQEKFKCIECGFELNADHNA |
| | AINIARSNNYVK |
| 48 | MPQGKKLVTAQIKLYLHRPVEPDNISWAQAGQILRDVSYETVLGLNHAITEWHLYERERARHYRE |
| | TKENLPAEERKKTWNRIYSEVRGLMKTASSGMASMVTRTAITRYQNSLKDIRSLKQSVPSYRLGH |
| | PILLREGGGETKLWRDNGNYMFRATLRNRSNEPTRLTFLLDTFKLEKSKKAVLDRVISGEYKLGA |
| | CQVAQDRRKRWFTRIAYSFPRPELKKDTSICVGVDLGLACPFYCAVNNGHDRLSCNEALIVERFR |
| | WQIRRRRRAFQNSLKFSNRGGHGRNKALAPLEKLAEKEINFRDTKYHQYTSRIIEFALKQNAGVI |
| | QIEGLEGFRASQQGILRDWAIADFHSKLKYKAEHAGIEVREIDARYTSQRCSECGNINEANRQSQ |
| | SDFLCTVCGYKTHADYNAARNISIVGIEKIIAEEIARKGLAEQEAQDVPQG |
| 49 | MVKRGEHMNTVRKIKIIINNENNELRKKQYKFIRDSQYAQYQGLNRCMGYLMSGFYVNNMEIKSE |
| | EFKTWQKGVINSANFFQEISFGKGIDSKSSITQKVKKDFSTALKNGLAKGERNINNYKRTFPLMT |
| | RGRDLKFKYDDNELDILINWVNKIQFKCVLGEHKNSLELQHTLHKVINNEYKIGQSSLYENKKNE |
| | LILILTIDIPTAKSSYEPIKDRILGVDLGMAVPVYMSINDNSYIKKSLGSYSEFAKVRKQFKERR |
| | NRLYKQLEACKGGRGRKDKLKAMNQFKEKEKNFAKTYNHFLSKNVVEFALKNKCEFIHLEKIESK |
| | GLENSVLENWTYYDLQEKIIYKAKREGIEIKFVNSSYTSQTCSKCNYVDKENRKTQVKFICKNCG |
| | FKANADYNASQNISKSKEFIK |
| 50 | MITVRKLKLTIINDDETKRNEQYKFIRDSQYAQYQGLNLAMSVLTNAYLSANRDIKSDLFKETQK |
| | NLKNSSSIFNDIPFGKGIDSKSSITQKVKQDFSIAIKNGLAGGERNITNYKRTFPLMTRGRDLKF |
| | SYKDDCSDEIIIKWVNKIVFKVVIGRKDKNYLELMHTLNKVINGEYKVGQSSIYFDKSNKLILNL |
| | TLYIPEKKDDNSIKGRTLGVDLGIKYPAYVCLNDDTFIRQHIGESLELSKQREQFRNRRKRLQQQ |
| | LKNVKGGKGREKKLAALDKVAVCERNFVKTYNHTISKRIIDFAKKNKCEFINLEQLTKDGFDNII |
| | LSNWSYYELQNMIKYKADREGIKVRYVNPAYTSQKCSKCGYIDKENRPTQEKFKCIKCGFELNAD |
| | HNAAINISRLEE |
| 51 | MNTVRKIKIIINNGDDEIRKSQYQFIRNAMYAQYQGLNRCMGYLMSGYYANNMDIKSQGFKDHQK |
| | TITNSLYIFNDIEFGKGIDSKSSITQKVKKDFSTALKINGLAKGERTVTNYKRSFPLMTRGRDLK |
| | FSYGDNDEILINWVNKIQFKAITGNSKNSIELEHTLHKIINGDYKVGQSSLTFNKKNELILILTI |
| | AIPEVKGDEYKPVANRTLGVDLGLAFPVYMALNDITYIRKSLGSYNEFAKQKLQYKARRERLYKQ |
| | LDSVKGGKGRKDKLKALDQFKEKEKNFAKTYNHFLSKKIVLFAIKNQCEYINLEKIDSSGLENRV |
| | LGLWTYYDLQKDIEYKAKLAGIKVRYVKAAYTSQRCSRCGDIEKENRQEQSKFVCKKCGLDINAD |
| | YNASINIAQSTEFIK |
| 52 | MGNINIGGENMKEFRTVKKLKLTIVADTKEEREEKYKFIRDSQYAQYQGLNLAMGILVSGFLKGN |
| | RKLDSEAFKQAQKEMMAIREQTFEDINFGKGITSSSLITQKVKADFKTALKNGLAKGERNVTNYK |
| | RTFPLMIKGNADSCYDQGKRKPLDFYYDNDDIYIRWCNGIIFKVVLGTRINENTTELKHTLHKIK |
| | GEYRVSQSSLQFDKNNNLIMNVNIRFKKELNTDFIEGRTLGVDLGLKYPAYVCLSDNTYIRKGLG |
| | SAEEFLKTRQQMKKRRTTLQHQLKLVKGGKGRNKKLKALEQFQNKERNFAKTYNHQLSCNIVKFA |
| | KENKCEFINLEKLTKEGFDNNILSSWSYYELQNMIEYKAERENIKVRYINPAYTSQKCSKCGYID |
| | KENRKTQSEFNCLECGLKLNADHNAAINIANSTEYIK |
| 53 | MITVRKVKLIVNSEEAEEINRTYKFIRDSMYAQYQGLNRCMGYLLSGYYANGMDIKSDGFKNHMK |
| | TIKNSLNIFDDINFGIGIDSKSAITQKVKKDFSTSLKNGLAKGERGATNYKRNFPLMTRGRDVKI |
| | SYLEDTNTFVIKWVNKIEFKVILGQKDNIELSHTLHKIINKEYTLGQCTFEFDENNKLLLALNIN |
| | IPDNLISKNKEIIPGRVLGVDLGVKVPAMICLNDNTFIKKSIGSYNEFFKVRSQFKARRERLYKQ |
| | LESSNGGKGRKHKLKATMQFRDKEKNFARTYNHFLSKNRIEFAQKYTCETINLEELNKKGFDNNL |
| | LGKWGYYQLQSMIEYKAERVGIKVKYVDPAFTSQTCSKCGYVDEENRITQDKFECQKCGFTLNAD |
| | HNAAINIARK |
| 54 | MPNITRKYQLKVVGDKEEIDRVYKYIREGTEAQNKALNEAMSALYAANLLDMSKDDKKELSKLFS |
| | RVINGKNESGFTDDICFATGLGTTSSIKQKVKQDFNNACKKGLMYGRVSLPSYKADNPLLVSKSY |
| | VQLLSESDKNFGIYNTYETPMDLVDALEKETNPEVYLKFANNILFKFVFGNPWKGREQRKVFERI |
| | FSGEYKICGSSIGIDGKKIILNLCMDIPKQKHNLDESIIVGVDLGLAIPAMCALNNDDYKRLSIG |
| | SIDDLLRVRIQLQNERRRIQGNLKNSKGGHGRQKKLKALENLKDRERNFVQTYNHMVSKRVVDFA |
| | VKNNARYINIEDLSGFWKTRYGKSKSEDEKVLRNWSYYELQNYITYKAQLHGITVRKVRAEYTSQ |
| | TCSYCGNKGIRKEQKKFVCVNPDCKCHKIYDGYINADFNAARNIAMSNDFAE |
| 55 | MKLVKTMRYQIIKPLSCDWDTLGTVLRELQRDTHSVLNKTIQLCWEWQGYSSEYKAANGTYPTPK |
| | DTLGRSLEGYVYDRLKVQFPKMYTPNLSQTIQRAMLKWSADSKAIFKGEVSIPSYKKDVPLDLRK |
| | DSIHIERRGHDYILSLGLVSRAYKKELGLPECQIEVLIGTPDKTQRVILARLLTGEYTVSGSQIV |
| | WDKRNRKWFVNLAYHFEARPEQLDKTKILGVDLGVVFPVYMAVADGHFRAGIPGGEIEEFRRRVE |
| | ARRRQLLRQGKYCGDGRIGHGRATRTRPLDKIADKIARFRDTINHKYSRYVVETARKLGCGVIQM |
| | EDLTGIREENLFLANWPYHDLQRKIEYKAREYGIEVRYVRPQYTSQRCSDCGYIHPDNRPEQAKF |
| | RCLACGFETNADYNAARNIATEGIEELIAAALNKASVV |
| 56 | MVKTMKFQIIKPINMLWKDFENILRQLQQDTRNIKNKTIQLCWEYQGFSSEYKEKHGEYPKHSDI |
| | LNYKTITGYIYDRLNNEYYRLNTGNLSDTIKSATDKWRNDIVDILKGEVSIPSYKRDAIIHVKNT |
| | NYKIVPEDGRYYLRLSLLSNVYRKELDLEKGQIDVAIRVADKNQKVTLERIFSGEYKQSSSQLMK |
| | KKNKWFFYMAFKFDPKTAKDLDPNNVMGIDMGITHPIYFAFNNSLKRGKIEGGEVESFRRRVEQR |
| | RRELLAQGKYCGKGRRGRGYETRVKPIKRIGDKIERFKDTANHKYSRYIVDVAVKNNCGIIQMED |
| | LKGISKNNIFLKNWPYFDLQTKIEYKAKEKGIVVKKIAPRYTSQRCSKCGYINKENRVSQDTFKC |
| | VKCDFGHKFYVNADYNAARNIATPGIEEIIKKQIEKQKEMDEWENETIKLPLVGSDK |
| 57 | MFMTKVTKVYLISEQIDKDGNKIDFKKISELLWNLQRQTRDIKNKCVQLCWEWLNFSSDYYKKSE |
| | EYPKEKDTLGYTLSGFVYDRIKNGSDLYSSNLSTSSRDTCTAFSNYKKEMLNGERSVLSFKANQP |
| | LDIHNKAIKLSYENGNFFVALKMLNRAGKEKYGINDDLRFRMQVRDKSVRTILERLMNDEYKVSA |
| | SKLMYDKKKKLWKLNLCYSFDNHVISTLDPEKIMGVDLGVVYPIMASVNGDYARFSIKGGEIEAF |
| | RNRVEARRRSLLNQSRYCGDGRIGHGRKKRTEPAAQIADKIARFRDTTNHKYSRALIDYAIKNGC |
| | GTIQMEKLTGITSNAEHFLKEWSYFDLQTKIESKAKEAGIKVVYINPKFTSQRCNKCGYIHTDNR |
| | PVQARFCCQKCGYEENADYNASQNIGTKHIDVIIEETLKMQCEPEVPTE |
| 58 | MKPMNKVVRLALICEHSDKDGNPVDYSDVYKLLWQLQAQTREIKNKTIQYCWEYSNFSSDYYKEN |
| | HEYPKEKDVLHYALDGFVNDKFKVGNDLYSSNCSYMTRKVCAEFKKSKSDFLKGTRSIISYKSNQ |
| | PLDLHNKSIRIEYKDNDFFAFLKLLKRPAFNRLGYKNSEIGFKVIVRDKSTRTILERCVDQIYGI |
| | SASKLIYNKKKKQWFLNLVYAFEPDNANNLDPSKILGVDLGIHYPICASVYGDLQRFTIHGGEIE |
| | EFRRRVESRKLSLLKQGKNCGDGRIGHGGKTRNKPVYSIEDRIARFRDTVNHKYSRALIDYAVKK |
| | ECGTIQMEDLSGITAESDRFLKNWSYYDLQSKIEYKAKEKGIKIVYIDPKYSSQRCSKCGHIDKE |
| | NRKTQSSFVCLKCGFEENADYNASQNIGIKDIDKIIENDLSSKCETDVN |
| 59 | MKITKTMKYEIDKSIDVPWKTFLSVLRDVQYAVWKTGNIAVKMTWDFQQEAWSYRQRFGEQLKFS |
| | DLGTGNKSQSTDIYQRACSEYPNVASSVLDATIRMAQDRYKTDAPDIYDGLKTIPYFKRELPIPI |
| | RAQQTKLTRKGTKRYVSFALLSKEGAKKSELPTRYNVQIRTGKGAREIFDRLVDGEYKLCDSKIL |
| | RKKGKWYLALSYSFEAENPQELDPKRVMGVDLGIVKTAYMAFNFDEYLRYEIEGGEISAFRGRIE |
| | SRRKSLLKQARYCGEGRRGHGRKTRMKPLEKLRDKVANFRRTKNHHYSKYIVEMAAKHGCGTIQM |
| | EDLSGINKRDKFLANWSYYELQSFVKYKAKERGIKVVLVDPNYTSQRCSCCGYIAEGNRKTQETF |
| | KCVICGYKTNADFNAARNIAEPRIKALIDAELKRQEKERKEAM |
| 60 | MSKVMKYELKYLGEEDFYEMQKMLWSLQEDTREILNKTIQIFFHWDYTNKESLETTGKALNLVEE |
| | TGYKDISGYVYDKLKSRYPDMSRGNLSATIRAASKKYRSSKVDILKGTMSIPSYKKDQPIILRPD |
| | GIRLHEREMIYTVELSLFSGDFKKKKAWKSNVLFQIKACDKAQKAIMQRLLSREYKLGESKLVYK |
| | KKKWFIYITYSFKKTDAKLDKNKILGVDLGVTYAIYACSIGEYGSFSIKGEEALEYAKRLEARTI |
| | SKQKQARYCGEGRIGHGIKTRLSTVYSTRNKLANHRDTLNHRYSKAVVDYAVKNRYGTIQMENLS |
| | CIKKNTGFPKRLQHWTYYDLQSKIEYKAAEQGIQVIKINPKFTSLRCSQCGCIHKDNRKTQESFQ |
| | CVECGYKDNADHNAALNISIPQIDLIIKEEMTSAKEK |
| 61 | MPIKALRVQIIKPFNTDYDSQPITWDELGRTLRDLRYAASKMANYVIQQNYMWEFFRQQYKQEHG |
| | SYPSVSEHKDKLYCYPRLTAMFPLAAGQMVNQIERHAKTVWSARKSEVLKLHQSVPSFKLNFPII |
| | VHHDSYRISEVPEDGKSSTHVFLLQANLLSREAATRTRYSFLINAGEKSKQTIVERIISGEYRQG |
| | ALQIVGDRKNKWYCHIPYEFKTEENNTLDPQCIMGIDLGISKAVYWAIIGSHKRGWIDGHEIEEF |
| | RRRVQARRKSIQEQGKYCGDGRIGHGRKRRLLPIEVLENRESNEKNTTNHRYSRFIIEAAIKNQC |
| | GVIQMEDLSGINERSTFLRNWTYYDLQMKIKAKAEEVGIEVRIVNPQYTSQRCSQCGHIDRDNRS |
| | NQATFVCTHCGYGGLYHCFACGKSQVEAGVCHLCGGETKIMKINADYNAARNLAICGIDQIIVQT |
| | LEGEGVR |
| 62 | MTQHIRVMQYQIVKPCNGDWSTLAKVLSYLQNATRQVLNKTIQLCWEWQGFGSDYKRKFGENAVD |
| | RELLGYSLFGFCYHQLRQEFPLIHSSNISQSIQRAVLRWNSDAPEILNGVKSVPCYKRGVGVDLH |
| | KDTVRLKRGKNNEFVVSMNLLSLIGRKEFGFKSAALDTVIKVADGRQRQIFSRVLSGEYGLGACQ |
| | IVCHKRKWFLQMRYKLAVKERQLNSNHILAVRMGIERPLYIAFSHTSSRVTLNGDNVTAFRKKFM |
| | ARYAGMIRQKTMQGGGNIGRGKAKRFERIESMRTKISGFQKSLNHKYSRIIVEQAVLNRCGTIQI |
| | ESPNVIRRQTAFLGNWAYFDLKRKVEYKAQQLGIKVVCLKTRDYEQRCSQCGHLNAVRETSKVLS |
| | GAYARSFFCESCGLLTTVDENAVQNLTLPNKPVMSQD |
| 63 | MALVKSVRIQISRCEELDYKRMSSIFRDIRYKTCRASNEAMRLFLLNAFQSIRYKELHDIYPDIK |
| | SLTGKSLNTYIYNSMKEIMDICQTGNVAQTQQFVKNRFNTDIKDLLVNEVSYTNFKKDMPLFLHN |
| | KSYTICKDEDSGKYKVECSLFNRQYQKENNIKRVTFVLGKMDGNQKATLDKIIAGEYKQGAGHIK |
| | QDKKKKWYFTISYSFEPQIRKINTNRILGVDLGMVNTAVLQIWDIDKQKWDWLEWKECMLDGGKV |
| | YNFNQRVEGMKRSLQRSRKVSSVTSPYSKGKKGRGVQARIKALNRLNHKISNFKDTINHQYSKYI |
| | IDFALKHNCGIIQMEDLSKIKDKAEEKFLAHWTYYDLQSKIGYKAKAHGIKVVKIKPAYTSLRCS |
| | KCGHIDKENRKEQAKFKCVKCKYKLHADVNAARNIAIPEIERLIEIEKEEVNAI |
| 64 | MTKNKMTTKTMKYEIRFEKALYNLLSDIQFEVFMLKNKATSIAYDWQNFSFSYHSRFGEYPKIKE |
| | LSGVTLTNDILGELREVQAKFVSSATVASSVKEAVEKFTSDKSKILKGEISIMRYKRDGSFPIRS |
| | QQINHLTKINSKTYTCKLSLLSREGAKEREMKNGQMDVELRTGKGAYEILDRIIEGSYKLCDSRI |
| | TKNKNKFYLLITYSFESDKVETLDENRIMGIDLGINIPAMLAISDNKYYREAVGDVTEISNFQKQ |
| | VESRKRKLQKQRKWCGEGSIGHGTKTRIKPLEVLSGKIKRFKDTKNHNWSRYIVDQAVKHNCGII |
| | QMEDLSGIAEENTFLKTWTYYDLQQKIKYKAEEKGINVVFIKPNYTSQRCSCCGHISKENRDVEK |
| | NGQDKFICVNCGYGSKFYVNADWNAAKNIATKDIENIIKEQLESQEKELKHNMKYAM |
| 65 | MDVGTVVKMTRCRIEQDDELYKVLDDLRYMIYRIKNKATSMAWDWEQFSFGYHERFGEYPVAKEV |
| | VGKNVSRDAYQHVKGLGEEFSSSFVDTAVEEASKHFKNHRSAILRGKEGIPVYRSDTSFTIRHTQ |
| | IKGLKKEQRDKYSGLFTLLSPKGSKVREQASTRYLFKIRSGGSSSAILDRILEGTYSLCDSKIVY |
| | EKKKRHYFLLVTYKFEAQKIEMNPERVMGVDVGFAVPATIAITDNPYACHFVGDAREVLAFEQRV |
| | LGTRRALYRSRSRAGDGSRGHGRKTLMKPTEVINSKVANFKATKNHEWSKFIVDYAVRHGVSTIQ |
| | LENLEKIAEESPFLKRWTYYDLQQKIAYKAKEYGIKVIRVSPDYTSARCNKCGAIHRKQERTLWR |
| | PKQSQFNCLHCHHRDHADRNAARNLAIPNIDQIIKAEKVEWTSYWTNVYKNPPA |
| 66 | MESEKEKYVVQSYGIKLIKPVGVDEGDWDFAGKVLRDLDYACFRVKNKAATRTYMNVIEKLEYEN |
| | IHGKGTYDKYFKTRYGKTFSAYNSECAREDKELNKGDLYREYFNLMAREGEKVVKYNMKKILNGN |
| | ASNITFKRNQPVPITSRMIFITKESGKYYAELTLLSPEKAKELGRKGKNGTRIKFLLSSKGQEKV |
| | ILDRLTSGEYDLRDSHIHVKKKGNKLTNYLIIAYRHKVKDDNDLIPNKVLGVDLGVSKAAYMAVS |
| | ESPVSEYINGGEIEQFRNGVEARRNGMRNQLKYHSSNRSGHGRATKLKPLEKLREKVSNFRKLTN |
| | HRYAKFIVDTALKNNCSIQMEELKGISKNDTFLKRWSYFDLQEKIENKAIAVGIEVKKVSPKFTS |
| | QRCNKCGYIDKESRKSQEKFECVNCGHKTNADLNAARNLSMLNVEKEIKAQCKAQKIKY |
| 67 | MLQVTKAVRFQIIKPLNFSWDEFGRILNDLSYHTTLMCNAAVQMYWEHNVMRNRYKAEHGRYPQD |
| | KEIYGQSFRNVVYHRLREMYPLMASSNVSQTNQFALKRWQTDLREVMRLQKSVPSFRLGTPVQVA |
| | NQNYSLYIAKGEPPEYCAEITLLGKDAACRRFTVLLDAGDAPKKAVFRRIVEGKYKQGVMQIIKH |
| | PRKKKWFCIVSYTITKDPAPGLDQERVMGVNLATGEAVYWAFSFSPKRGSIPAGEIEAAEKKIRA |
| | ITARRREMQRTAGVTGHGRKRRLKATRVLAGKTANIRDAINHKYSRRIVRIAAANRCGKIRLADM |
| | SALGMSGALKAWPWSDLVQKIGYKAAEQGIDVEIVEKPGDRAKAWHTCSECGYSAPENVGDNTEF |
| | LACKECGARISLEYNAALNIAVLARDSIPEQQTSAS |
| 58 | MEITKATRYQLIKPLDVSWEDFGQILRDLSYHTTKMCNAAVQLYWEYHNQRLAYKQEHGKYPEDK |
| | VMYGMSFRNVVYHRLRGIYPLMASSNTSQTNQFALNRWKNDVPDVMRLQKSIPSFRLGAPIQVAN |
| | ANYRLYVAEGEKPEFRADVTLLGKDAAQGRFSLLLDGGDAPKKAIFRHIVDGTYKQGVMQIVRHP |
| | RKKKWFCHISFTFTSEEKSLDESRAMGVNFGSGEALCWAFNFGPKRGTIPAAEIEAAEAKIAAIT |
| | ARRRGMLRTAEARGHGRTRRLKPTESLQGQAANIRDAINHKYSRKIVSVAVGNRCGIIRLADTSG |
| | LELDGAFKHWPWAGLAEKIRYKAEEAGIVVETADAKKAFYTCSKCGYSHPENTDGNKEFLTCKNP |
| | DCEAQVNLQYNTAKNIAVNVPGPEEKKSPKKEKRKKEIAKQ |
| 69 | MTKAKDRELSKVFRVEVLKPLNLTWDEFGGLLRRTQYHAAHLANEVITTQYLLAKGKLERNGSFC |
| | ALVAHACHDCSLHADVKCTVCKWARDKFKADARRILRADISLPSYKNNLCMIKNRSVKLRETPDG |
| | WAARLAILPKADGKNQVQPEVLLRTEEMKRRSPGAYQVLERIASKEYKQGTTQVKRDTRTGKIYL |
| | LISYSFRPERESGLEASRVMGVDLGVSTPAYCAFNDSLKRKSLLIEGRKLLKTKWQIEGRRRDIR |
| | RHNDQRDLRRGHGKEAKFRPMEAVEQHWVDFRQSWNHVLARRIIEYALSNHAGAIHLEDLSPGTN |
| | SKFLGRNWPVAELLDYIEYKAKERGIAVKRVNPFKTSQTCSDCGAIKESFTFGDRKKAGFPDFVC |
| | DACGFRTHADYNAARNIAKAP |
| 70 | MNKCIKVILNKCINIDIKEAKKIIKNMSYLSCKASNKAIDMWKQHSLNIMELKSNDKNFNQKEYE |
| | QATYGKNYKNVIEGYMKEIMNICNTSNVSTLHQQQVQNDWKRLRKDVLNYRANLPTYKLDTPCYL |
| | KNNNYKLRNHNGYFVDISLFSMKGLEQIGQKKGYQLQFEIDKMDGNKKSTINKLINNGYKQGSAQ |
| | LKISDKGKIELIMSFSFEAKESNLDENRILGIDLGIVNVATMAIWDGNTQEWDWVNYKHNILNGQ |
| | ELIRFRQKLFNMGMSEFEMQNEVYKQNQKIHQKQLNKHNIGAIDGLELVKYRDTIDKKKREMSIA |
| | SKWVGEGRVGHGYKNRMKPLEKIRNKASNFADTFNHKYSKYIVEFAIRGNCGVIQMEDLSGATKN |
| | THGKFLKDWSYYDLQTKIEYKAREVGIDVIYVKPQYTSKRCSKCGNIHTDNRDCKTNQAKFKCMN |
| | VTCGHEENADINASKNISIPYIDKIIEEYIKENDIYKNKK |
| 71 | MIQNRRQNRLILTRKIQIIPLGEKEEIDRVYKYLRDGIFYQNKAMNQYMSALYIAAIKDISKEDR |
| | KELNRLYSRVSNSKKGSAYDKSIEFAKNMNLGYVVKQVKQDFANSCKNGLLCGKVSLPTYRKNNP |
| | LLVHVNFVRLRSTNYHQDNGMYHNYESHTDFLDHLYSKDLEVFIKFANNITFKMIFGNPHKSAYL |
| | RSEIQQIFEENYKVCGSSIQIDGKKIILNLSMDIPKQELELDENIVVGVDLGLAIPAMCGLNIND |
| | YIRQSIGSKDDFLRIRTQLQSQRRRLQKSLASTSGGHGRQKKLKPLEKLKDRERNFVKTYNHYVS |
| | KNVVDFAVKNKAKYINVEDLSGFDSNQFILRNWSFYELQQFITYKAAKYGIEVRKINPYHTSQIC |
| | SCCGHWEEGQRIDQAHFKCKSCGAELNADFNASRNIAMSTDFV |
| 72 | MATKEKRIVKVMRLRILKPAGEMSWSQLGKLLRDTRYRVFRLANLAVSEAYLNFHLWRTGRSQEF |
| | KADDMGKLSRRLRQMLADEGVAADELDRESPTGAVPDAVSGPLFQYKIRAITNKNKWREVIRGTA |
| | SLPTFRLDMAIPVRCDKARMRRLERMENGDVQVELTICRKPYPRVVLQTGDIGGGQEAILARLLD |
| | NTGNDLTGYRQRVFEIKQERQTSKWWLYITYDLPAPQTGKADPDVVVGVDVGYAVPLYVAINNGH |
| | ARLGWRQFDALGRRIRKLQTQVLARRRSIQRGGRVNISHATARSGHGVKRKLLPTEILQRRIDKA |
| | YQTLNHQLSASVIDFARDHGAGVIQVEDLEGLKEELTGTYIGARWRYHQLHQFLKYKAEENGIEF |
| | RAVNPRFTSRRCSKCGHINVAFDRAYRDAHRENGKTARFICPQCGFEADADYNAARNLATLDIEA |
| | LIEAQCARQGLTKDAL |
| 73 | MITVRKLKLSIRESDEESRKVKYQFIRDSQYAQYRALNLAVGILSSAYLKSNKDTKQESYKNAIK |
| | SLINSNPIFSEIEFGRGVDTLSLVTQRAKKDFKNSIKNGLARGERNLTSYKRTYPLMTRGRDLKF |
| | RYEGDDIVIRWVNKIEFNVITGSNKIKENTVELKHTLHKVINKEYNVKESSLMEDKNNNLILNLT |
| | LDIPNELSYQPIEGRTLGVDLGIATPAYVCLSDDTYVRKGIGSIEDFLKVRTQFQKRRRVLNQSL |
| | VMAKGGKGRKKKLKALESFKEKERNFAKTYNHQLSHEIVKFAKNHKCESINLEKLIKEGFNNRIL |
| | RNWSYYELQSMIEYKAEREGIKVRYVNPAYTSQKCSKCGHVDKANRQTQAQFKCVECDFELHADH |
| | NASINIARSEEFVQGAGS |
| 74 | MVKRGEHMNTVRKIKIIINNENNELRKEQYKFIRDSQYAQYQGLNRCMGYLMSGFYVNNMDIKSE |
| | EFKTWQKGVTNSANFFQEISFGKGIDSKSSITQKVKKDFSIALKNGLAKGERNINNYKRIAPLMT |
| | RGRNLKFKYDDNELDILINWVNKIQFKCVLGEHKNSLELQHTLHKVINNEYKIGQSSLYFNKKNE |
| | LILILTIDIPTAKSSYEPIKDRILGVDLGMAVPVYMSINDNSYIKKSLGSYSEFAKVRKQFKERR |
| | NRLYKQLEACKGGRGRKDKLKAMNQFKEKEKNFAKTYNHFLSKNIVEFALKNKCEFIHLEKIESK |
| | GLENSVLANWTYYDLQEKIIYKAKREGIGIKFVNSSYTSQTCSKCNYVDKENRKTQAKFICKNCG |
| | FKANADYNASQNISKSKEFIK |
| 75 | MITVRKIKLTIMGDEETRNRQYKWIKDEQYNQYKALNIGMSYLATHLFLKMSESGLEQKTEKNIK |
| | TIEKQISKIENNITKEESKKKVNEEKLNNLINELDTLNASLSKLKEELEEISNNRSNVDDTFKRM |
| | YVDDLYNALSKVPFQHSDMKSLVSRKVELDENTDMKDLMSGNRSVRNYKRNHPLLVRGRDLRFRY |
| | DGSNIKIKWIQGIEFKAILGKISKTIELRHILNKVIDGEYKVCDSSLEFNKNNHLILNLAIDFPY |
| | TNKIEFIEGRVVGVDLGIAVPAYVALNDIAYVKKSIGDIDDFLRVKTQMKKRRNLQINLTSVKGG |
| | KGRSKKLKALDRLSEKESNFVRTYNHFLSKSIVKFAIDNKAGQINLELLSENALSDKIIKNWSYY |
| | QLQQFIKYKAERYGIKVKYVDPYRTSQTCSVCGHYEEGQRSKQDIFTCKNEKCKMFEKEVNADYN |
| | AARNIAISTKYIDDIKESEYYYKKSF |
| 76 | MSIPLTRKIKLSVYVPNTITEEKERHEYKTFVWQLLRKVNQQNYEFHNLLVKKLIELDSVLEGRL |
| | TEDDNFIEIRNRYYSDRKDKKNQREFFDYIRMKKDELIKEFGGKNLHGYIYTYLKNYIKSLPEEQ |
| | QFMASYTYGSISKNVVDKYTNDQFDVFRGVKTIATYKNTQPIPINIIGHVKKNEKGELYKNTGKE |
| | WFNKYDDIYTFKFKSIKKHEIELQLNFGKDRSNNRIIVDRIYNNDPNYKICDSKIQVVGTEIFLL |
| | LVFKQFVTKRELGLDVRKVIGVDLGVKNVATIATNFSDHIEIIGEGEFVLMRTKLQIEKQKRSIQ |
| | RNSKYSRGGHGRNRKLQKLNEYRNYERNFRSTFNHKISKDVIEIAIKHGAGQINIEDLSSIPLKE |
| | KNNRILRFWSYYDLIQKITYKAKREGIIVNLINPSYTSQKCHQCGQIGNRPKQDTFLCTNPTCKA |
| | FNEPINADVNAAKNIAKISL |
| 77 | MQRSITLKILRPRDEKISWEEMGYLLGGLSMKVCRMSNFCMTHHLLHALKLETELLNPRGDLYCY |
| | PVLAEEYPEVPSGIICAAETRARKLFKRSAAKVLRSETSLPSFRKDSSIPIPVAGYRILQDGDGN |
| | YCAEIQLISRQGAKTQKLPGRICLVLADNWRDKSAKSALQKVAAGKVRRGVATLFRAKKDWYLCI |
| | PYVTEPADIGENFEPGLVMGVAFGMFDVLAYGENTLLKRGAISGEEVLSHQDKFMARRKKIQEQY |
| | AWSGRKGQGREDALKPLRHLYEVEKNYRDLVNNRYAKWVVDIAVKNRCGEIHLDSGNSTSKGNKE |
| | ILLSHWSLYDLKDKICRKAEEKGIRVTECNVPNLRTRCSHCGTEQAVENRKRMFLCKNCGYGTTD |
| | KNKSNGYISADYNAARNLAVYDTGDTEPV |
| 78 | MIITRKIAITIVSEEAQESYNYLRQQIYYYYKALNFGMNHIYFNYVAKEKIKLADSAYKEREEKY |
| | INAIHIAKEKLQKDLSVSQRAQAEKSLEVNTNNLDKLRKAISKDAKETFQKVMGAVERTNVTDAI |
| | KKEFPMLQRDSIDFAASKVASDFNNDLKLGLMTGSRTLRIYKRNQAYPFRSRRLKFYKENGDFFI |
| | NSSKSLLFKCLLGVKRQNSKELIQILEKILDNQYKICDSSLEFNKKKLILNLCIEVDENTHSENM |
| | KVPGVVKGRIVGVDLGIQIPAYCTLNDSPFKKKAIGSVDDLLRIRTQMQARRRRLSKNLISARGG |
| | KGRGKKLKALDRFEEYERNYVRTYNHFISKQIIQFTLQNQAEQINLELLQMEHTKSKSILRNWSY |
| | YQLQQMIEYKAKREGIVIKYVDPYHTSQVCSKCGHFEENQRMDQNTFRCKKCKYRTNADYNAAKN |
| | IANSTRYISSIQESEYYNIKNRNIAK |
| 79 | MIITRKIAITIVSEEAQESYNYLRQQIYYYYKALNFGMNHIYFNYVAKEKIKLADSAYKEREEKY |
| | INAIHIAKEKLQKDLSVSQRAQAEKSLEVNTNNLDKLRKAISKDAKETFQKVMGAVERTNVTDAI |
| | KKEFPMLQRDSIDFAASKVASDENNDLKLGLMTGSRTLRIYKRNQAYPFRSRRLKFYKENGDFFI |
| | NSSKSLLFKCLLGVKRQNSKELIQILEKILDNQYKICDSSLEFNKKKLILNLCIEVDENTHSENM |
| | KVPGVVKGRIVGVDLGIKIPAYCTLNDSPFKKKAIGSVDDLLRIRTQMQARRRRLSKNLISARGG |
| | KGRGKKLKALDRFEEYERNYVRTYNHFISKQIIQFTLQNQAEQINLELLQMEHTKSKSILRNWSY |
| | YQLQQMIEYKAKREGIVIKYVDPYHTSQVCSKCGHFEENQRMDQNTFRCKKCKYRTNADYNAAKN |
| | IANSTRYISSIQESEYYNIKNRNIAK |
| 80 | MNDRSIITRKLTLIPAFSDRPKWEEKVMSYTEQFYIDKIAYYKNKLTKTKGKEEKQKIKDKLASL |
| | EEQENEFEESGILTQANVIDYTYDLVRNAMASEANRKNAHISYIILELLHNGGQTMDFNARNKLI |
| | NDLVNYGLRVKGSSKGSLFDELDIENPLNAYGFAFKQDLKKKIRDMVNSKRVLDGKSSVITYKAD |
| | SPFSINKENMSFTHDYSSFEELSDHIRDNDTNLYFNFGSSGNPTIARFKINLGAGRHKKNKDELI |
| | ATLLKLYSGEYQFCGSRIGIEKNKIILNLVLSIPKKVRALDENTVVGVNLGVAVPAMCALNNNEY |
| | ERLAIGSADEFLRVRTKLQAQRRRLQKSLKDASGGHGRTKKLKALERVAKAESHFANTYCHMISK |
| | RIVDFALKNNAKYINLENLTGYDTNDFILRNWSYYKMQQYTTYKAEKYGIIVRKVNPCYNAQACS |
| | VCGNYAPGQRKSRAVFICANPACKSHKKNHGKLDAEFNNARNVAMSTLYMNDGQVTEKSFKEARD |
| | YFGIEEEIETI |
| 81 | MITARKVKLTITENREDGYNFIHNELREQNQALNMAMNHLYFNYVAREKIKLADETHKIKLAEDQ |
| | GYLDQKYTELKEVKTDKKKQNIRKSIQAAKKRLETLRKAENKQVAEKFKEIIAASEKTNLRDFIT |
| | DNFNLTSDTKDRLTQKVSADFKNDIVDVLRGERTLRRYKKGNPLYIRGRNLTFYIKDEEYYIKWM |
| | KSIVFKCVLGVKKQNSLELQKTLDKVIEGKYKVCDSSIEFKQNSLILNLTLNIPVCNSFDKVEGR |
| | VVGVDLGMKIPAYVTLNDSDYIRRAIGSIDDFLKVRTQMQSRRRNLQRALKSTKGGKGREKKLKA |
| | LNQFEVKEKNFAKTYNNFISSNIVKFASDNKAKQINMEFLSLSETQNKSVLRNWSYYQLQQMIEY |
| | KANRVGIKVKYVDPYHTSQICSKCGHYEEGQREKQEVFICKNPECKNFNIEVNADYNASRNIAKS |
| | NKYITKKEESEYYKIN |
| 82 | MILTRKIKLVIVSENREEGYNLIRTEIREQHKALNLAYNHLYFEHNAIQKLKQNDEDYKQKRNKL |
| | QELINKKYEEHQKAKNLEKKEALREAYNNKKQELYNFEKEYNEKARQTYQQVVGFTQQTRVRNLI |
| | NRECNLMSDTKDGITSKVTQDYKNDCKAGLLIGKRSLRNYKKDNPLLVRGRSLKFYKEDGDYFIK |
| | WNKGTIFKCILHIRKKNVVELQSVLENVLLGAYKVCDSSIGENNKDMILNLSLNIPDKETQGYIP |
| | GRVVGVDLGLKIPAYLSLSDKVYVRKGIGSIDDELRVRTQMQKRRRRLQKSLAAVKGGKGREKKL |
| | KALDHLKGKEANFAKTYNHFLSTQIVTFAVKNQAGQINMEFLEFDKMKNKSLLRNWSYYQLQIMV |
| | EYKAKREGIIIKYVDAYLTSQTCSKCDHYEDGQREKQENFMCKNCGLEVNADYNASQNIAKSTSY |
| | ISDSTESEYHKKKQQVLKEILGENDIMNEQLSLENNCDDIA |
| 83 | MSKITRKIKIIPDIDGITHEESNKKCYNTFYKFDRKLYKVANLLVSQLYGLDSLLSLMRLQNDEY |
| | VKCLSKLSFKSITDATKEEIKKRMKEIDAELISIKNDIAPKHPQTYSYRAVTSSEYAKDIPSDIL |
| | NNLKQDVYQHFNENKKEQIRGERSLATYKKGMPIPFNLKKKHGIISVGDNYYLPWFEDTRFRLNF |
| | GRDRSNNRAIDNCIKTKKYKLCAAAKIQLKERKLFLLITVDIPKAESVPVKGKVMGVDLGVVNPA |
| | YVAVNDGPERSRIGNGEAFQKQRDVFRRRFRELQRSQLTQGGHGRKHKTKATEILRGKERNWVQT |
| | ENHRISREIVNLASRWKVETIQMESLKGFGKNQEGEVEYNHKRLLGRWSYFELQKDIEYKAAMAG |
| | IAVQYVNPAYTSQTCHVCGQRGNRIERDTFICTNPECTCYNQAQDADMNAAINIAKSKDVIK |
| 84 | MITVRKLKILIDGESRNESYKFIRDSMYAQYLALNKAMSYLGTAYLSRDKEIFKEAIKSLNNSNP |
| | IFDNINFGKGIDTKSSVNQTVKKHIQADIKNGLAKGERSIRNYKRDYPLMTRGRDLKFFYCDINS |
| | TKVKVKWVNGIIFDVMLGKEYNKNDLELRSFLNRVINKEYKISQSSICFDKHNRLILNLSVNITD |
| | NIPNEVVKGRIVGVDLGMKIPAYVTLNDSEYIGKPIGDINDFLKVRKQFKERKERLQKQLAINKG |
| | GRGITNKMQLMDAFINKEKNFANTYNHGVSKAIINFAKKYKAEQINVEFLALAGSEKEILSSTIR |
| | YWSYYQLQQMIEYKANREGIAVKYVDPYLTSQTCCKCGNYEVGQRINQELFECKLCGNKMNADRN |
| | ASFNIARSTKYISSKEESDFYKQLK |
| 85 | MQRSVTLKIIRPEDETISWEELGYLLRGLSFKVCRMCNFCMTHQLLHALKLETELLNPQGNLYCY |
| | PRLAEEYPDVPTGIICAAETRARKVFRRSAEAVLHSETSLPRFRKDSSIPVPVAGYKILQDSDHN |
| | VYADVQLLSRQGAKTQKLPGRIRLVLADNWRDQSAKAALRQIADGKVKRGVASLFRVKNDWYFQI |
| | PYVTEAVNTGEGFEPDLVMGVAFGLQDALVYAFNTSLKRGAVSGEEVLAHQEKYAARRKKIQEQY |
| | NWSGRKGHGREDALKPLRHLYETERNYRSLVNSRYAKWVVDIAMKNRCGMIHLDSANYVSSGKKI |
| | LLSRWPLYDLKEKIRRKAEEKGIQVTECSIPNLRTRCSLCGKEQEPEGEKHTFVCKDCGYGKADK |
| | NRRSGSITVDYNAARNLAAYKSEDTKL |
| 86 | MITVKLQLYKPTKCKEQRLFSHIREFTSCANWYLDKLQESRTTSRVKIHNGYYETARKSFRLLSA |
| | NVQLALDKAIETQRAFLNKKGKKSVPKFKKQFACFRQDTFKIFDRYVQFNMPGRERVNIPFKVCN |
| | PNHKQFIKQQPKRSQLINKKGKWFLYVSYETDKPAIDGNINIIGVDLGVKKIVTVSNPEASVNVF |
| | FSGNKAIYTRNKYQRYRKQIQRAKDTGKAKRGYRALKRISGKEKNWIKDTNHKISKQIVNIAKQN |
| | KADIAIENLKGIRERIKATKKVRRMLHSWSFRQLISFLQYKSAMAGVRIVSVDPRHTSQRCPRCG |
| | HISKDNRKSQSAFKCSQCHYSVNADLVGSRNIALTALNLYGEGKRPSERAVMLLPMAEGKTLMAS |
| | CLEAPSVRAG |
| 87 | MPTITLRLELHNPTKVKQDMYERMTEVNTAFTNWLLNHPKLNQATSKIFKEFSPQRFPSAVVNQT |
| | IREVKSQKKNQKTKKFQTLWCCFNNQNVKVEKKGSFYTVSFPTLEKRIGVPVVTRPYQEAWLNRL |
| | LDGTAKQGAAKLYKKRKKWYLAIAITFDVKPRHETKVMGVDVGLRYIAVASVGTKSLFFKGSQCA |
| | FIRRRYAALRRTLGKAKKLQMIRKIGRKESRWMKDQNHKISRQIVNVALANGVGVIRMEALTGIR |
| | KRAKSAKEAGRSLHAWAFHQLQTMIAYKAEMAGIRVEWVDPTYTSQTCKCGHREKANRNGIRFRC |
| | QRCGYTLHADLNGAINIAKAISGFAAEPSALVTGAPPIGVHRNPMGRGDDTPLKLLRCPNQKWMR |
| | TQTTQESHAL |
| 88 | MWHVELKRTARVKLAIPDDRRDDLKRTMLTFREVAQRFADRGWERDEDGYVITSRTRLQSLVYKQ |
| | VREDTGLHSDLCIGAVNLAADSLRSAVERMKAGKNVGKPTFTVPTATYNTGAVSYFTDGDGTGYC |
| | TLAAYGGRVRAEFVYPPDEDCPQRQYLGGDEWEPKGATLHYERDDGEYYLHVTVERDEPETELGE |
| | AENGTVLGVDLGVENIAVTSAGAFYSGGLFNHRRDEYERIRGSLQQTGTESAHRTIEKMGDRERR |
| | WNTDVLHRISKAIVQEAITHDCSHIAFEDLTDIRDRMPGAKKFHGWAFRQLYEYVEYKAAEFGIA |
| | TTQVDPAYTSQRCSKCGTTLRENRTSQAAFCCQKCGYEVHADYNAAKNVATKLLRSGQKSPAGGA |
| | INQLALKSGTLNGNGDFTPASS |
| 89 | MPTITLRLELYNPTKVKQDMYERMTEVNTAFANWLLNHPELNQATSKIFKEFSPQRFPSAVVNQT |
| | IREVKSQKKNQKTKKFQTLWCCFNNQNVKVEKKGSFYTVSFPTLEKRIGVPVVTRPYQEAWLNRL |
| | LDGTAKQGAAKLYKKRKKWYLAIAITFDVKPRHETKVMGVDVGLRYLAVASVGTKSLFFKGSQCA |
| | FIRRRYAALRRTLGKAKKLQMIRKIGRKESRWMKEQNHKISRQIVNVALANGVGVIRMEALTGIR |
| | KRAKSAKEAGRSLHAWAFHQLQTMIAYKAEMAGIRVEWVDPTYTSQTCKCGHREKANRNGIRFRC |
| | QRCGYTLHADLNGAINIAKAISGFAAEPSALVTGAPPIGVHRNPMGRGDDTPLKLLRCPNQKWMR |
| | TQTTQESHAFRRAECQIISSQTRVPRPDGSAGSTMSRRGRRWRISCT |
| 90 | MKASRFLILNDYEKNGILSSPQGGESMPTITLRLELYNPTKVKQDMYKRMTEVNTAFANWLLNHP |
| | ELNQATSKLFKEFSSQRFPSAVVNQTIREVKSQKKNQKTKKFRTLWCCFNNQNVKVEKKGEFYTV |
| | SFPTLEKRIGVPVVTRSYQEAWLNRLINGTAKQGAAKLYKKRKKWYFALAITVEVQQREETKVMG |
| | IDLGLRYIAVASVGTKSLFFKGSQCAFIRRRYAALRRTLGKAKKLHMIRKIGRKESRWMKDRNHK |
| | ISRQIVRFALANGVGVIRMEELTGIRKRETSAKEAGRSLHSWSFHQLQTMIAYKAEMAGIRVEWV |
| | KPTYTSQTCRCGHREKANRNGIHFQCKKCGYTIHADLNGAINIAKAISGFAAEPSALVTGAPPIG |
| | VHLNPMGRGDDTPLNLGVVRIRNG |
| 91 | MANKNVNDSKIKTHILTKKVQLIVDTDREDTDEEKKAEVDRVYKYLRDSMRCQSREMNQYYMHLW |
| | MMSVARNLNDDRYSMKKYMNNIIDAVHPYLDKNNKEFNNKQKKITRDFEKKIKNLCEEYQAETEI |
| | LNNDTLRELNKCFNRKKDGAYDTNFVNEMPEGLGIVMGRTVEQDFQNDCKAGLLSGIRNPRSYKI |
| | NYPLIIPKSFVAYGVGSGKAMQGRGIFPDMEYSDFHNMLFSTKNPNITYNFVHDIDFKLVFGSMK |
| | RSHELRVIFDRIKMGEYSICGSTIEINNKKKIMLNLSYEAPIYEKPNLDENTVVGVDLGMAIPAV |
| | CSLNNDDRTYKYIGDSHELEFIKKGIQAQRRSYQRNAVYNKGGHGRNRKLENLDRLKKRERNTTR |
| | THNQRYAKQIVDFALANNAKYINLEKLKGFSNNEKNKLVLRNWCYYELQQYIEIDASKYGIKVRY |
| | IYPMNTSRTCSVCGTLTTDEDVKNGVGRVSQDEFICKDPNCPSHTLYTRGPKGHKVPYFNADRNA |
| | SRNIAMSEDFVKKNAKDNAFKKIDDLYEINNDIDAA |
| 92 | MQLNTITLTVKLQIRPLGDKEAQNEVWRNLRNINRDVFKAANLLMSHLHFVEAFEHQFMQTDRIL |
| | EKEMLDRENKKKNKKLNAQQIETIAQEIAEIKQKRKELKIEVAEKVKIFYNGKNESFAYNFIRHE |
| | YPQIPSYSVSILVKTVQNKFKEEWKEVKKGEKSISNFKKTIPIPIDDKQIANTDKGKKLCYIKKE |
| | GEKFVWELNSLEAKFEIVTGKYKQSKYMGEDKNKRTTLERVMNFEYKVADSSLKIEENKIFLLLV |
| | CKIPQQQATSLPNVSIGVDLGIRTPACIACNDGLRVISLGSKQDFLAIRQRFYEHRKRLQKSLAM |
| | TKGGKGREKKLKALEKLRKAERNWVRTYNHTLSKKIIHFAIQCRASRIQIELLEGFGRNENKVDE |
| | KGNYLLGKWSYYELQNMIKQKAEQYKLVVTTIDPYHTSKTCHVCGELGYRKGADFFCQNERCKEY |
| | QKRQNADHNAAFNIAKSTTFVAKKEDCTYFKLEQEKKSKKEEE |
| 93 | MRRTVKIKLSLNQNQQKLLQETIKQFKQACQRVVDYGWNRNGLKTYQKNKLHKATYSEIREKTDL |
| | PANLVIRARDRASETIKACVKKIKNGEKASKPTFKSDSIVYDKRTLTVWLEEERCSIATTNGRIK |
| | ADFVLPNEYNDYYDKYLNNGWKITQSTIEKHSYEDDEPFYLHLGLEKEIQKNQSVNPTIMGIDLG |
| | IENLAVTSTGRFYSGTELFSRRERYEEVRGKLQAKGTRSAHLTIKQMSGRENRFACDTLHRISKK |
| | IVEEAISKDVDVIAMEELEKIRQKISNNKKFQTWAFKKLQEYIEYKANERGIEVKFVDPKYTSQR |
| | CSRCGTTLKQNRINQHFECKDCGYRVDSDYNAAKNIGFKTILDGQMSQSRMGNGQLALKSGVLKP |
| | NGNYYSYPN |
| 94 | MIVTRKIQIRPINKEHYKILEDYLRTCRLIANKSQTLFYVYWQEIMANNLKGKNIDAYFKERYQH |
| | SFKQTIYHILRPKHLEIPSRITDDTLNIAYKDFCNDLKNGLLKGERSLRTYNNGWIPVRSQTTKI |
| | TKQNNKYILEWLKGIKLEVYFGRDKSGNQVVVDRILNGQSKFCDSKFIKKEKKWFLLLCVNEPEK |
| | ENNLFDDISIGVDCGINIPAVCAVNKGYGRAYIGSSVLLTRFRMQKDQRQRQKNYISSSGMHGRQ |
| | KVDRAIFKAQDFEHRYMHTFHHKISKEVIKFALKNRASKIIMEDLKRFGQNQEESEEKKKIRRFW |
| | GYQQLQSMIEYKAKLENIAVKYIPPAYTSQTCSQCGYTDANNRKQSKFVCLNPKKKCGFEANADY |
| | NAALNIAKGGIKKVNNKDV |
| 95 | MPTITLRLELHNPTKVKQGMYERMTEVNTAFANWLLXLELHNPTKVKQGMYERMTEVNTAFANWL |
| | LNHPELNQATSKIFKEFSSQRFPSAVVNQTIREVKAQKKKQKAKKERTFWCCFNNQNVKVEKKGV |
| | FYTVSFPTLEKRIGVPVVTRSYQEAWLNRLLNGTVKQGAAKLYKKRKKWYLAVAITVEVQQREET |
| | KVMGVDLGLRYIAVASVGTKSLXAKLYKKRKKWYLAVAITVEVQQREETKVMGVDLGLRYIAVAS |
| | VGTKSLFFKGNQCAFVRRRYAALRRRLGKAKKLHMIRKIGRKESRWMKDQNHKISRQIVAKKLHM |
| | IRKIGRKESRWMKDQNHKISRQIVRFAVANGVGVIRMEALTGIRKRATSAKEAGRSLHAWAFHQL |
| | QTMIAYKAEMAGIRVEWVNPTYTSQTCKCGHREKANRNGIRFRCQRCGYTLHADLNGAINIAKAI |
| | SGFAS |
| 96 | MILTRKIKLVIVSENREEGYNLIRTEIREQHKALNLAYNHLYFEHNAIQKLKQNDEDYKQKRNKL |
| | QELINKKYEEHQKAKNLEKKEALREAYNNKKQELYNFEKEYNEKARQTYQQVVGFTQQTRVRNLI |
| | NRECNLMSDTKDGITSKVTQDYKNDCKAGLLIGKRSLRNYKKDNPLLVRGRSLKFYKEDGDYFIK |
| | WNKGTIFKCILHIRKKNVVELQSVLENVLLGAYKVCDSSIGFNNKDMILNLSLNIPDKETQDYIP |
| | GRVVGVDLGLKIPAYLSLSDKVYVRKGIGSIDDFLRVRTQMQKRRRRLQKSLAAVKGGKGREKKL |
| | KALDHLKGKEANFAKTYNHELSTQIVTFAVKNQAGQINMEFLEFDKMKNKSLLRNWSYYQLQIMV |
| | EYKAKREGIIIKYVDAYLTSQTCSKCDHYEDGQREKQENFMCKNCGLEVNADYNASQNIAKSTSY |
| | ISDSTESEYHKKKQQVLKEILGENDIMNEQLSLENNCDDIA |
| 97 | VITARKVKLTITENREDGYNFIHNELREQNQALNMAMNHLYFNYVAREKIKLADETHKIKLAEDQ |
| | GYLDQKYTELKEVKTDKKKQNIRKSIQAAKKRLETLRKAENKQVAEKFKEIIAASEKTNLRDFIT |
| | DNFNLTSDTKDRLTQKVSADFKNDIVDVLRGERTLRRYKKGNPLYIRGRNLTFYIKDEEYYIKWM |
| | KSIVFKCVLGVKKQNSLELQKTLDKVIEGKYKVCDSSIEFKQNSLILNLTLNIPVCNSFDKVEGR |
| | VVGVDLGMKIPAYVTLNDSDYIRRAIGSIDDFLKVRTQMQSRRRNLQRALKSTKGGKGREKKLKA |
| | LNQFEVKEKNFAKTYNNFISSNIVKFASDNKAKQINMEFLSLSETQNKSVLRNWSYYQLQQMIEY |
| | KANRVGIKVKYVDPYHTSQICSKCGHYEEGQREKQEVFICKNPECKNFNIEVNADYNASRNIAKS |
| | NKYITKKEESEYYKIN |
| 98 | MMTKSVKIKLGTPIDSNWNTVRKILADLRYNSSKMLNFSIQQCYQWMIYRNEYREKYGKYPRAKD |
| | IYGYSHRNHIYRQAAEIFPIFNRGNISQTVGMATSRWSSDQKDVMSLRKSIPSYRLQAPIYIANQ |
| | SYTISRTENGLVVDCSLVSKKYAKEETNDKTRYRISLVVKDNSTETILDRIVSGEYSQGYGQIVK |
| | DRRKEKWYLLVAYRFEPKKTKETGRILGIDLGIVYPLYMALNDSHHRYRIDGGEIEHFRRNIEKR |
| | KNQLLDQGKYCGDGRRGHGIRTRIAPIEFAREKIKNFRNTTNHKYSKFVVDIAQKHDVETIQLEK |
| | LDGISEDSTFLKNWSYYDLQQKIQYKAQEKGIKVAYIDPRYTSQRCSRCGNISRENRQDQQRFRC |
| | TKCGFSANADYNAAKNISTQNIEKIIEEELKK |
| 99 | MTTKVMRYQLIEMVDNEKRFMYKMLDDLRYEVFKISNRAIQMFWDIDNTSYAFKQKFQENLDLKE |
| | LTGVKSLAYISRALKEEYQKLNNTSVEQVSRKVEKEWKKNKSNMITADTSMIRYKRKNANIKLKN |
| | TQFKIEPLDNNFYRISARLLSKSYAKDLHENGFEFAQKFKEKGKKKEKTIKEFIKKNDDNMWVHF |
| | KIKAHDGSQKSIIERVVNKEYKVGGSDIYCDRKRKYYLNLSYTFEAEQAKVDENKILGIDVGVNT |
| | PATLAISDDKWYKEFIGDKQEIENYRNQVESRRRRLQKNAALYSGEGSTGHGRKTRLKSVDKIRD |
| | KIARFKDYKNHIWSRAIVNEAIKHGCGTIQMEDLTGIAANTNEKFLKTWSYFDLQTKIQYKAEEV |
| | GIKVVKVKPAHTSARCNNCGHIHSKENKDKWRPKEFHHEKFICQNCNHTAHADLNAAKNIAMKDI |
| | EKIIKDQLESQEKYYKNQMKYILD |
| 100 | MITTRKFKLAIVSDNRNEAYNFIRSEIRNQNKALNVAYNHLYFEHIATEKLKHSDEEYQQHLTKY |
| | QEVASNKYQDYLKVKEKAKASKDDEKLQKRVDKAREGYNKAQEKVYKIEKEFNKKSKETYQKVVG |
| | LSKQTRIGKLVKSQFTLHYDTEDRITSTVISHFNNDMKTGLLRGDRSLRTYKNTHPLLVRARSMK |
| | FYEENGDYYIKWIKGIVFKIIISAGSKQKANIGELKSVLINILDGHYKVCDSSISLNRDLILNLS |
| | LNIPVSKENVFVPGRVVGVDLGLKIPAYVSVNDTPYIKRGIGNIDDFLKVRTQLQSQRKRLQKAL |
| | KSTSGGKGRSKKLKGLDRLKAKEKNFVNTYNHFLSKNIIQFAVKNNASVIHMEELHFDKLKHKSL |
| | LRNWSYYQLQTMIEYKAEREGIEVKYVDASYTSQTCSKCGHCEEGQRVLQNAFICKNKECKGYGH |
| | KVNADFNASQNIAKSTDIIRGTEIAKTNDTAKNTKSIKGNQQSDDEVERKQLELELN |
| 101 | MSDTSPYKHQTKNIGLLVHPEVSKATSKIFKEFSHGKFPSAVVNQTIREVKSKKKKQNAKSFKKL |
| | WCCFNNQNLKMEKVGDFYTVSFPTLEKRIGVPVVARPYQQAWLERILNGTVKQGASELYRKKKEW |
| | YIAIPITFEVEQRETKVMGVDLGLRYIAVASVGTKSLFFKGNQVAFVRRLFAARRRKLGKLKKLS |
| | AIKKSKDKESRWMKDQNHKISRQIVDFALTNGVGIIRMEDLTEI*NRAKSKKEAGRNLHSWAFYQ |
| | LQKMIKYKAEMVGICFELVKPDYTSQTCKCGHREKANRIGIQFRCKKCGYTCHADLNGAINIAKA |
| | HSGLVAPSVLVTGTPPMWVHSNHMGRGDDTPLNLGVVQNGNGLRTLTTQESHGFSRVECQYGRII |
| | CWTWTKTVY |
| 102 | VITVRKIKLTIMGDEETRNRQYKWIKDEQYNQYKALNIGMSYLATHLFLKMSESGLEQKTEKNIK |
| | TIEKQISKIENNITKEESKKKVNEEKLNNLINELDTLNASLSKLKEELEEISNNRSNVDDTFKRM |
| | YVDDLYNALSKVPFQHSDMKSLVSRKVKLDFNTDMKDLMSGNRSVRNYKRNHPLLVRGRDLRFRY |
| | DGSNIKIKWIQGIEFKAILGKISKTIELRHILNKVIDGEYKVCDSSLEFNKNNHLILNLAIDFPY |
| | TNKIEFIEGRVVGVDLGIAVPAYVALNDIAYVEKSIGDIDDFLRVKTQMKKRRNLQINLTSVKGG |
| | KGRSKKLKALDRLSEKESNFVRTYNHFLSKSIVKFAIDNKAGQINLELLSENALSDKIIKNWSYY |
| | QLQQFIKYKAERYGIKVKYVDPYRTSQTCSVCGHYEEGQRSKQDIFTCKNEKCKMFEKEVNADYN |
| | AARNIAISTKYIDDIKESEYYYKKSF |
| 103 | VKLVKTMRYQIIKPLSCDWDTLGTVLRELQRDTHSVLNKTIQLCWEWQGYSSEYKAANGTYPTPK |
| | DTLGRSLEGYVYDRLKVQFPKMYTPNLSQTIQRAMLKWSADSKAIFKGEVSIPSYKKDVPLDLRK |
| | DSIHIERRGHDYILSLGLVSRAYKKELGLPECQIEVLIGTPDKTQRVILARLLTGEYTVSGSQIV |
| | WDKRNRKWFVNLAYHFEARPEQLDKTKILGVDLGVVFPVYMAVADGHFRAGIPGGEIEEFRRRVE |
| | ARRRQLLRQGKYCGDGRIGHGRATRTRPLDKIADKIARFRDTINHKYSRYVVETARKLGCGVIQM |
| | EDLTGIREENLFLANWPYHDLQRKIEYKAREYGIEVRYVRPQYTSQRCSDCGYIHPDNRPEQAKF |
| | RCLACGFETNADYNAARNIATEGIEELIAAALNKASVV |
| 104 | VEGEKEKYVVQSYGIKLIKPVGVDEGDWDFAGKVLRDLDYICFRVKNKAATRTYMNVIEKLEYEN |
| | VHGKGTYDKYFKTRYGKTFSAYNMEQAKEDKELNNGSFLREHFDSMAREGEKVVKNNMKKILNGS |
| | ASNITFKRNQPIPIRSRMIFITKESGKYYAELTLLSPEKAKELDRKGRKGTRIKFLLSSKGQEKV |
| | ILDRLTSGEYDLRDSHIHVKKRGSKLNNYLIVAYRLKVKDDKDLIPNKILGVDLGISKAAYMAVS |
| | DSPVSEYINGGEIEQFRNGIEARRNSMRNQLKYHSSNRSGHGRSTKLIPLEKLRAKVNNFKELTN |
| | HRYAKFIVDTALKNRCTIIQMEDLSGISKTDTFLKRWSYSNLQEKIENKAKTKGIEVKKVSPKFT |
| | SQRCNKCGYIDKESRKSQEKFECVNCGHKTNADLNAARNLSMLDIEKVIKAQCKAQKIKH |
| 105 | VPIKALRVQIKPENTDYDSQPITWDELGRILRDLRYAASKMANYVIQQNYMWEFFRQQYKQEHGS |
| | YPSVSEHKDKLYCYPRLTAMFPLAAGQMVNQIERHAKTVWSARKSEVLKLHQSVPSFKLNFPIIV |
| | HHDSYRISEVPEDGKSSTHVFLLQANLLSREAATRTRYSFLINAGEKSKQTIVERISGEYRQGAL |
| | QIVGDRKNKWYCHIPYEFKTEENNTLDPQCIMGIDLGISKAVYWAIIGSHKRGWIDGHEIEEFRR |
| | RVQARRKSIQEQGKYCGDGRIGHGRKRRLLPIEVLENRESNEKNTTNHRYSRFIIEAAIKNQCGV |
| | IQMEDLSGINERSTFLRNWTYYDLQMKIKAKAEEVGIEVRIVNPQYTSQRCSQCGHIDRDNRSNQ |
| | ATFVCTHCGYGGLYHCFACGKSQVEAGVCHLCGGETKIMKINADYNAARNLAICGIDQIIVQTLE |
| | GEGVR |
| 106 | MQKVLKVQLICEHFDQEGNAVDYKTICKLLWELQKQTREIKNKSIQYCWEYSNFSSDYYKEHHEY |
| | PKEKDILSYTLGGYVNDKLKAGNDLYSANCSTTIRTACAEFKNAKSDFLRGDKSIISYKANQPLD |
| | LHNKSIRLEYQNGTFYFWLKLLNKSAVKANGFRNTEIRFKALVKDNSTRTILERCAASVYDIAAS |
| | KLLYDRKKKCWYLNLVYAFEPQLAKALDPEKILGVDLGIHYPICASVFGDLKRFTIDGGEIEAFR |
| | RRVEARKISMLKQGKNCGEGRIGHGIRARNKPVYAISDKIARFRDTMNHKYSRALIDYAVKNGCG |
| | VIQMEKLTGVTADANRFMKNWTYFDLQTKIEYKAKEAGIQIIFIDPHYTSQRCSKCGYIDRENRP |
| | VQSRFSCQKCGFTENADYNASQNISIRNIEKLIEAQLQTKCESQADNS |
| 107 | LATKCIKLAGEYAKENSLEKDKFFKELRDIQYKTWLACNRAITYYYSNDMQNFIQKDVGIPKEDD |
| | KLLYGKAFKSWVANRISEILGDGISSYATDCISQFVSNRYKNDKKAGLLKGNVALSQFKRDIPVM |
| | LRERAYSHIDTPKGLGIEISFFSKTKQQELGIKRILFTFPKIDGSSKSILTRIMDKTYKQGSIQI |
| | TYNKRKKKWMFAISYTFENKLEKVLNDNLVMGIDLGITKVATMSIYDIEKHQYKNMCFKEQTIDG |
| | TELIHYRQKIEARRKSLSISSKWASDNATGHGYKRRMKKANNIGDKYNRFKDTYNHKVSRYIVDL |
| | AYKHGVKTIQMEDLSGFSEHQSESLLKNWSYYDLQNKIKYKAEEKGINTIFINPQYTSKRCSKCG |
| | NIHEENRDCKNNQAKFECIICGHKENADINASKNIAIPYIDKIIKEYIKDTI |
| 108 | MKITKQTKIRFNFQSPNAETNRAVYEKLNGCIYLTWKAFNRAYNEFIFQHINKQKKGENYTAEEN |
| | KKFRNSGYRSILDIDIHSSIKASISQKAYKQFLNDTKKGGVLSGSRTWSSYKLPSPIPFQIRMLS |
| | IFKDTDGYFMRIPYFSKDFPFNLMIERNEQKVIVDRILSKEYELNDSSIQKDKIHNKWYINLCYS |
| | FDKEPDSSIDKSIVVGVDLGIAIPVMCAINSSEYIRGSFGNRQEIDNFRARIKSKRWQILKQNNS |
| | FYDLRTGHGKSGKLKPLIPLEDRIKKFMNTYNHKLSHAIVAFALQNKAGIINFENLENLSEVKQK |
| | NMYLRDWNNADIITKTEYKAKEQGIEVHFINPAYTSQRCSKCGHIEKENRESQSEFKCTKCGYEA |
| | NADFNAARNISQMNDKSSLKIDT |
| 109 | MQRSVTLKIIRPEDEKISWEELGYLLRGLSFKVCRMCNFCMTHQLLHALKLETELLNPQGNLYCY |
| | PRLAEEYPDVPTGIICAAETRARKLFRRSAEAVLHSETSLPRFRKDSSIPVPVAGYKILQDADHN |
| | VYADVQLLSRQGAKTQKRPGRIRLVLADNWRDQSAKAALQQIAAGKVKRGVASLFRVKNDWYFQI |
| | PYVTEVVNTGEGFEPDLVMGVAFGLQNALVYAFNTSLKRGAISGEEVLAHQEKYAVRRKKIQEQY |
| | NWSGRKGHGREDALKPLRHLYETERNYRSLVNSRYAKWVVDIAVKNRCGMIHLDSANYVSSGKKI |
| | LLSRWPLYDLKEKIRRKAEEKGIQVTECSIPNLRTRCSRCGKEQEPEGEKRTFVCKDCGYGKADK |
| | NRRGGFISVDYNAARNLAVYKSEEKEL |
| 110 | MAKGTLSKVMKYELSYLDGCGDFQNMQKELWALQRQTREILNRTIQIAYHWDYTDREKFKKTGQH |
| | LDVKSETGYKRLDGYIYDELKEDVKNFASVNVNATIQKAWSKYKSSKTDVLRGDMSLPSYKSDQP |
| | LVLHGQSMKLSEGEDGVVMQATLFSNTYKKEQEYSNVRFAVRLHDTTQRTIMKNILSGDYGLGQS |
| | QIVYKRPKWFLYLTYNFSPKQHEADPDKILGVDLGETIAIYASSIGEYGGLRIEGGEVRAFAKQL |
| | EARKRALQKQATYCGEGRVGHGTKTRVADVYKAEDKIANFRNTVNHRYSKKLIDYAIQHQYGTIQ |
| | MEDLTGVKKDTGFPKFLQHWTYYDLQQKIEAKAKEHGIRIIKVNPAFTSQRCSKCGNIDSGNRPS |
| | QAVFCCTKCGFKANADFNASQNISIPGIDKIIKESYGANME |
| 111 | MNKVVRIYLISEHTDKKGDPVNYQDINKLLWELQKQTRTIKNKTIQYCWEYQNFSSDYYKEHHAY |
| | PSEKEILSYTLDGYVNDKLKNSSDLYSVNRSSTIRNAIKEFKNAKADMIKGVKSVISYKSDQPLS |
| | LHNQSVRIEYIGGQYFANIKLVNSPYAKEHDFASTMIRFKFWIRDKSAETIIQRCLSNEYKISES |
| | EMFYDRKKKQWYINLCYSFSASKNDSLDIHKILGVDLGIAYPMCASVYGDYARFTIHGGEIEKFR |
| | RTVEARKLSMLKQGKNCGEGRKGHGIKCRNKPAYNISDKIARFRDTINHKYSKALIDYALKNNCG |
| | VIQMEELTGITADADRFLKNWTYYDLQTKIKYKAEENGIKFKLIKPKYTSQRCSKCGYIDKENRK |
| | TQAHFLCLKCGFECNADYNASQNISIENIDMIIEQELKSDANIGCT |
| 112 | MTKVVRVYLIEQKDKNGDIVEYTKINKLLWDLQKQTRIIKNKAIQYYWEYKNFSRDYCEKNGNYP |
| | SVEEILTYKTVDGYINNRLKIDNDLCAINRSSTIKHAIAEYKNAESDIKDGTRSIINYKSDQPLD |
| | LHNTSIHIERVKEEYYLFANMVSREYAKANNFANARICFKLMLKNNKSAQTIIDRCLNGDYKISE |
| | SKIYDRKKKQWCINLAYSFSPSNVQQLDYNKILGVDLGITYPLCASVYGEYDRLAIHKGEIENFR |
| | NKVEARRYSMLRQGKNCGDGRIGHGIKCRNKPAYNIGDKIARFRDTTNHKYSRALIEYAIKNNCG |
| | TIQMENLAGITDKAERFLKNWTYYDLQTKIQYKAKECGIKIQIINPQYTSQRCSRCGYIHSDNRK |
| | TQENFLCLKCGFAANADYNASQNISIKDIDKIKKNWKICEPDIYRIT |
| 113 | MPTITRKIELTLCTEGLSEEQRKEQWGLLYHINDNLYKAANNISSKLYLDDHVSSMVRMKHAEYL |
| | SLLKELARAEKQKTPDADAIAELRKKVAAAETEMTDQEHAICKYATEMSTETLAYKFATEIETHV |
| | FGQILTCLKQAAQSNFNSDAKDVKRGERAIRNYKKGMPIPFPWNRSLKIEADGGNFYLRWFNGLR |
| | FLLNFGKDRSNNRLIVKRCMKMDADYEGEYKLCNSSIQIAKREGKTKLFLLLVVSIPQEHVELNK |
| | KIVVGVDLGINVPAYVATNITEERKAIGDREHFLNSRMAFQRRYKSLQRLKGTAGGKGRTKKLEP |
| | LERLRKAEHNWVHTQNHLFSREVVDFAVKAHAATIHMEDLSGFGKDNDGNADEKKEFVLRNWSYY |
| | ELQNMIAYKAAKYGIKVEKIRPAYTSKTCSWCGQQGFREGVTFICENPACKQCGEKVHADYNAAR |
| | NIANSKAIIKKNE |
| 114 | MPTITRKIELTLCTEGLSEEQRKEQWGLLYHINDNLYKAANNISSKLYLDDHVSSMVRMKHAEYL |
| | SLLKELARAEKQKTPDADAIAELRKNVAAAEKEMTDQERAICKYATEMSTQSLSYRFATELETNI |
| | FAKILDCLKQGVFATFNSDARDVKRGERAIRNYKKGMPIPFAWNDSLRIEKDNKDFYLRWYNGLR |
| | FLFNFGKDRSNNRLIVERCLKMDADYDGEYKLCNSSIQIAKREGKVKLFLLLVVSIPQEHVELNK |
| | KIVVGVDLGINVPAYVATNITEERKAIGDREHFLNTRMAFQRRYKSLQRLKGTAGGKGRTKKLEP |
| | LERLRKAEHNWVHTQNHLFSREVVDFAVKTHAATIHMEDLSGFGKDNDGNADERKEFVLRNWSYY |
| | ELQNMIAYKAAKYGIKVEKIRPAYTSKTCSCCGQQGFREGVTFICENPECKQYGEKVHADYNAAR |
| | NIANSKEIIKKNE |
| 115 | MPTKCIKVALEFIKKDNNISEKQINKELKDIQFKTHLACNRAMTYMYSNDSETIIQRDIGIPKED |
| | DKFLYGKSFGSWIENRMNEIMDGVLSNNVAQTRAFVINTYNQDKKNGLFKGNVTLSQFKRDMPII |
| | LHNKAFKIIETSKGLGVEIGLFNLKKQKELGIKRIVFLTPRLGESEKSIFKRLMDKSYKLGTAQI |
| | SYNQRKRKWMIAISYTENKEKDTIYLDNNKVMGIDLGIVNVVAMSIYDNAKEQYIKMSWKDRLIS |
| | GTELISYRQKLESRRKNLAIASKWASSNRCGHGYKNRMRAVNKQGDKFNRFKDTFNHKISRYIVN |
| | MALKYHAGIIQMEDLSGFSNEQSESLLKNWSYYDLQEKIIYKAKENGIEVILIDPKYTSKRCSEC |
| | GNIDDKNRDCKKDQEHFKCTACNYTDNADINASKNIAILKIDSIIENYLKNKNKG |
| 116 | MIKIVKTMKYEIKYDKELYNLLSDIQHAVWLIKNRATTAAYDWQQFSFAYNERFGEYPKEKDVIG |
| | KTLAPDVYGFLKEIGSFVSSSIVDSAVQEAITKFKNDKVKILKGEQSIQTYRRNGSFPIRASQLK |
| | GLTKLDNKTYNAKLSLLSNEGAKERDCKGQFLVTLVTGNGAYEILDRVINGEYKMCDSRIYKRKN |
| | KFYLLLTYKFEKETDKVLDENRIMGVDIGVAVPAVLAINEDKFYRQYVGDAKEVSDFVAQINDRK |
| | KRLQRSRKWAGEGSRGSGRKKLMKPVDAISNKIHNYRETKNHTWSRFIVNEALKNECGTIQIEDL |
| | SGISKDNAFLKEWTFYSLQQKIIDKAKEHGIQVVKVKPNYTSQRCNKCGFIHKDVNKEIWRPTQS |
| | SFKCLNCGHETNADLNAARNIAMKDIEKIIVDQLNVQEQHQKHAEKYLV |
| 117 | MGAKIVKLQLIYKSDEVMPYKDYCKELFALMREMSIIKNKIATYLYLDKRFSVALPIEKDTKVKS |
| | ASGLASAFASELLVNYKTGEVEVLKNHSSNRSAAGQDVQAKFKKFLKESMGAYAVNPPSFKDNGT |
| | LCLHDRCIQIYYDEDNKDYGAKLFLLSHSYAKELGIKSSNGFDYKLLVGDDSSRANIERIIAGEY |
| | KISASNLKWDKRKKKWYLLLCYSFTEKRTTSYDPKEYENNVMGINFGVICPMYMSFNHCKNRYNI |
| | EANEIEAFRKQVEARRIALRRQRKYCGDGSIGHGKNKRNSPAMKIDDKIARFRDTCNHKYARYAV |
| | DMAIKHQCGIIVIEDLTSIAEKEERLFLKTWSYYDLQNKIEYKAKEAGIKVIKINPQYVSRRCSK |
| | CGYISFEKEENIKAPDYRKFHCVECGFQSHVDYNASQNNATIGIEAIIAEQIKEHNSENVKNQPA |
| | AKAAKSKSKIKETA |
| 118 | MITSRKIKLAIVSDNKDTAYSFIREETRNQNRALNVAYTHLYFEYVAQEKLKQSDKEYQQHLEKY |
| | KNAAAKKYQEFLTIKEKSKSDENLQPKMDKVRETYNKAMEKVYKIEKDYSKKAREIYQQSVGLAK |
| | QTRLGKLIKSEFDLHYDTVDRIGSNAMSDFSNDRKSGILSGERSLRNYKKTNPLMVRARSMKLYE |
| | EDNNFYIKWINDIVFKIISAGSKQRMNIAELKSVFIKLLSGQCKMCDSSISLDKGLILNLSIDMP |
| | ITKENVFIPNRVLGVDLGLKIPAYVSLNDTHYIKGAIGNIDDFLKVRTGLQSQRRRLQKSLQSTG |
| | GGKGRRKKLQALERLKTKEKNFVNTYNHFLSKNIVQFAVKNNAGAIHMEELKFDKMKNKSLLRNW |
| | SYYQLQTMVEYKAKSEGIEVYYVDASYTSQTCSKCGNLEEGQREARDTFVCKKCGYNVHADYNAS |
| | QNIAKSTKAINKTIEITNIV |
| 119 | MTKVTKVYLISEQIDKDGNKIDFKKISELLWNLQRQTRDIKNKCVQLCWEWLNFSSDYYKKSEEY |
| | PKEKDTLGYTLSGFVYDRIKNGSDLYSSNLSTSSRDTCTAFSNYKKEMLNGERSVLSFKANQPLD |
| | IHNKAIKLSYENGNFFVALKMLNRAGKEKYGINDDLRFRMQVRDKSVRTILERLMNDEYKVSASK |
| | LMYDKKKKLWKLNLCYSFDNHVISTLDPEKIMGVDLGVVYPIMASVNGDYARFSIKGGEIEAFRN |
| | RVEARRRSLLNQSRYCGDGRIGHGRKKRTEPAAQIADKIARFRDTTNHKYSRALIDYAIKNGCGT |
| | IQMEKLTGITSNAEHFLKEWSYFDLQTKIESKAKEAGIKVVYINPKFTSQRCNKCGYIHTDNRPV |
| | QARFCCQKCGYEENADYNASQNIGTKHIDVIIEETLKMQCEPEVPTE |
| 120 | MIKVVKTMKYEIMYDKELYDLLSEIQYAIWLIKNRATPAVYDWQQFSFSYNERFGEYPKEKDVLG |
| | KTLAPDIYGFLKELGSFVSSQIIDTAVQEAIKKEKNDKMKILKGEQSIQIYRRNGSFPIRASQLK |
| | DLTKINNKTYEAKLSLLSNAGAKERDCKGQFLVKLVTGNGAYEILDRIINGEYKMSDSRIYKKKN |
| | KFYLLLTYKFEKETEKVLDENRIMGVDIGVAVPAVLAINEDKYYRQYVGDAKEVSDFVAQINDRK |
| | KRLQRSRKWAGEGSRGNGRKKLMKPVDAISNKIHNYRETKNHTWSRFIVNEALKNECGTIQIEDL |
| | SGITTDNAFLKDWTFFSLQQKITDKAKEHGIKVVKVKPNYTSQRCNKCGFIHKNVGKEIWRPTQS |
| | SFKCLNCNHVTNADLNAARNIAMKDIEKIIEVQLKAQEQNAKHEEKYLV |
| 121 | MPKQISKVMKYELKYLGEEDFYEMQKMLWSLQEDTREILNKTIQIFFHWDYTNKESLETTGKALN |
| | LVEETGYKDISGYVYDKLKSRYPDMSRGNLSATIRAASKKYRSSKVDILKGTMSIPSYKKDQPII |
| | LRPDGIRLHEREMIYTVELSLFSGDFKKKKAWKSNVLFQIKACDKAQKAIMQRLLSREYKLGESK |
| | LVYKKKKWFIYITYSFKKTDAKLDKNKILGVDLGVTYAIYACSIGEYGSFSIKGEEALEYAKRLE |
| | ARTISKQKQARYCGEGRIGHGIKTRLSTVYSTRNKLANHRDTLNHRYSKAVVDYAVKNRYGTIQM |
| | ENLSCIKKNTGFPKRLQHWTYYDLQSKIEYKAAEQGIQVIKINPKFTSLRCSQCGCIHKDNRKTQ |
| | ESFQCVECGYKDNADHNAALNISIPQIDLIIKEEMTSAKEK |
| 122 | MAEHTVITRKIEVHLHRSGDSEEAKELLREKYHMWDTINDNLYKAANLIISHCFENDAYEDRLRI |
| | QSPRFKQIQDSLRNAKRNKLGESEIKELKSEREQLFDEFKKQRYTFLRGGVAEGPNPEQNSTYRV |
| | ASDNFLDTIPSDILTCLNKNITTTYKSYRKEIEFGNRTIPNFKKGIPVPFPIKKDKKLRISKRDD |
| | GSIFIKFPGRLEWDLDFGRDRSNNREIVERVLNGMYDVGDSSIQETRSGKRFLLLVVKIPKVKNV |
| | SLDSNRVVGIDLGINTPLFAALNDNDYERVSIGSRDQFLNVRNRMNAQKREMQKNLRSSTTGGRG |
| | RRHKLQALERLEGKERNWVHLQNHIFSKGAIEFAIKNNAGVIQMERLTGFGRNANDEVENDRKFL |
| | LRNWSYFELQQLIEYKADAAGIEVRYIDPYHTSQTCSFCGHYEAGQRVDQAHFICKNPECEKGKG |
| | KKNDDGTYAGINADWNAARNIARSNKIVDRKKK |
| 123 | LILTRKIQIIPLGEKEEIDRVYKYLRDGIFYQNKAMNQYMSALYIAAIKDISKEDRKELNRLYSR |
| | VSNSKKGSAYDKSIEFAKNMNLGYVVKQVKQDFANSCKNGLLCGKVSLPTYRKNNPLLVHVNFVR |
| | LRSTNYHQDNGMYHNYESHTDFLDHLYSKDLEVFIKFANNITFKMIFGNPHKSAYLRSEIQQIFE |
| | ENYKVCGSSIQIDGKKIILNLSMDIPKQELELDENIVVGVDLGLAIPAMCGLNINDYIRQSIGSK |
| | DDFLRIRTQLQSQRRRLQKSLASTSGGHGRQKKLKPLEKLKDRERNFVKTYNHYVSKNVVDFAVK |
| | NKAKYINVEDLSGFDSNQFILRNWSFYELQQFITYKAAKYGIEVRKINPYHTSQICSCCGHWEEG |
| | QRIDQAHFKCKSCGAELNADFNASRNIAMSTDFV |
| 124 | MKTKKHTKVMRYEIIKPLDSTWEVFGQVLRQVQYETRHALNKSIQLSWEWQGFSAEYKRQSDHYP |
| | NLKEITNYTNLQGYAYNTLKHEFPSLYRGNFSQTVKRATDKWNSDRGEILRGERSIPNFKKDIPI |
| | DVVAAAFKEGKEQNLRIRKILSSEEYNNETGKRIQGYTVKVNLISDSYKKELGRNSTAFEMLIKV |
| | GDNTQKAIIERLIEGKYNIAASQILYQKGKGSGKKGKWFLNLSYSFEKEDVTLNPDLIMGIDMGI |
| | VHPIYIAFSDSYARYHINKGEINRFRNQIEKRKKELLHQGTYCGEGRKGHGIQARIKPIEVISDK |
| | IANFRKLCNHRYSKFVVDIAVKHGCGTIQMEDLQGISKDDVFYKNWSYFDLQEKIAYKAKESGIK |
| | VIKVNPRYTSQRCSQCGNIDSENRVNQADFLCTACGFKALADYNAARNISTRNIEKIIDKAVGKE |
| | IIDVVDLEYGNIS |
| 125 | MVKVVKIYLISEQVDEQGKDVDYNTICGVLWDLQWETREIKNKTVQLCWEWSGFSSDYYKKYGEY |
| | PKEKNLLDYTMGGFVYDKLKSKYHLYTANLSTTSQNTCGIFRTYKVDFVKGNRSVLSFKADQPLD |
| | VHKKSISIDRIDDNYFVKLKLLNKSGIQKYGIRDDFHFRMLVKDNSTKTILERCVGGDYKAAASK |
| | IIYDKKKKMWCLNLSYEFDVNTAKDLNKNRILGIDIGIVYPVVASVNGELDRFVIQGGEIETFRR |
| | RVENRKKSLLKQTKYCGDGRIGHGRNKRTEPVDIISDQIARFRNTANHKYSRAVIDYAVRKQCGT |
| | IQMENLKGITDKSDRFLKNWSYYDLQQKIEYKAKEKGINVVFINPKYTSQRCSRCGYIDSANRPK |
| | LPNQSKFLCIKCGFTENADYNASQNIALYNIEKLIDAEA |
| 126 | MPTRVMKYQIVKPMNCDWKLLNRHLLDLQQEAHQILNKTIQLCWEWQGFCSEYKAKYDIYPVDKD |
| | IFHLTLAGYVNRMLRLQFPKSNSNNLGTSRIKAINRWHTDLKDVKSGQKSIASFKADVPIDLHYL |
| | SIKLHKEKDCYYADLSLISNIYKKELGRESGKLLVLLKSGDEVSRDILRNCLDGIYKIRASDISH |
| | KKNKWFLNLHYNYEETNKSLDKDRILGIDMGIAYPVYMSVYNTQITSYIEGGEIERFHKQVEKRS |
| | AEFQRQGKYCGNGRIGHGVKTRLKPLSFATDKIANFRETTNHKYSKYIVDFAVKNNCGNIQMEDL |
| | TGIKNERIFLRNWTYFDLQKKIRYKAEENGINVLLIKPHYTSQRCSQCGDIDKKSRITQENFTCI |
| | SCGYQTNADHNASINISTPNIENIINDFMSLKID |
| 127 | MFMTKVTKVYLISEQIDKDGNKIDFKKISELLWNLQMQTRDIKNKCVQLCWEWLNFSSDYYKKSE |
| | EYPKEKDTLGYTLSGFVYDRIKNGSDLYSSNLSTSSRDTCTAFSNYKKEMLKGERSVLSFKANQP |
| | LDIHNKAIKLSYENGNFFVALKMLNRAGKEKYGIKDDLRFRMQVRDKSVRTILERLMNDEYKVSA |
| | SKLMYDKKKKLWKLNLCYSEDNHVISTLDTEKIMGVDLGVVYPIMASVNGDYARFSIKGGEIEAF |
| | RSRVEARRRSLLNQSRYCGDGRIGHGRKKRTEPATQIADKIARFRDTTNHKYSRALIDYAIKNGC |
| | GTIQMEKLTGITSNAEHFLKEWSYFDLQTKIESKAKEAGIKVVYINPKFTSQRCNKCGYIHTDNR |
| | PVQARFCCQKCGYEENADYNASQNIGTKHIDVIIEETLKMQCEPETPTE |
| 128 | MNKVMRYQIIKPIDIDWKTFGDILNKMRQEVRFTKNKTIALYNDWLTYCFQYKNEHNEYPKLINY |
| | CGYKVFSGYAYDKFKTDVVFSNTANYTTSVREACSAYDTHKTDILKGNCSIPSMGANQPIDLHNK |
| | SLSVDVNESGDYIATISLLSNRGKKEFGLKSGQIKVVLKAGDKSSKDILQRCVSKEYKICGSKII |
| | YKGKKTFINLCYGFEPEASDLDKSKVMGIDLGVSVPAYMAFNFDKYKRDSIKDNRIMTTKWMMDQ |
| | QLSIAKQSCKYLSDGNSGHGRKKKMICYDKYSNKSRNLSQTINHGWSKYIVDVAFRNGCGTIQME |
| | DLSGVTSEKDKFLKNWTFYDLQQKIEYKAKEKGIDVVKINPRYTSQRCSECGCICKRNRPDQKTF |
| | KCISCGNSSNADFNAAKNIATIGIEDIIANTEVIE |
| 129 | MSIAVKVMKYQIVCPVNVEWKVFETYLRTLSYQSRTIGNRTIQKIWEFDNLSLNHFKETGEYPSA |
| | QHLYGCTQKTISGYIYDQLKEEYQDINKANMSTTIQKTLKNWNSRKKEIWRGEMSIPSFRNNLPI |
| | DIHGNSIQIIKEKSGDYIASVSLFSSKFIKENDLPNGKILVKLSTRKQNSMKVILDKIVNSIYAK |
| | GACMLHKHKNKWYLSITYKATIKEEHKFDEELIMGIDMGKINVLYFAYNKGLVRGAISGEEIEAF |
| | RKKIEYRRISLLRQGKYCSENRIGKGRKKRIKPIDVLNDKVAKFRNATNHKYANYIVQQCLKYNC |
| | GTIQLEDLQGISKEQTFLKNWTYFDLQEKIKNQANQYGIKVVKIDPSYTSQRCSECGYIHKKNRQ |
| | DQSTFECQQCSFKIHADYNAAKNISVYNIEKVIQKQLELQEKLNLTKYKEQYIEQMENIN |
| 130 | MTYLSIAVKVMKYQIVCPVNVEWKVFETYLRTLSYQSRTIGNRTIQKIWEFDNLSLNHFKETGEY |
| | PSAQHLYGCTQKTISGYIYDQLKEEYQDINKANMSTTIQKTLKNWNSRKKEIWRGEMSIPSFRNN |
| | LPIDIHGNSIQIKEKSGDYIASVSLFSSKFIKENDLPNGKILVKLSTRKQNSMKVILDKIVNSIY |
| | AKGACMLHKHKNKWYLSITYKATIKEEHKFDEELIMGIDMGKINVLYFAYNKGLVRGAISGEEIE |
| | AFRKKIEYRRISLLRQGKYCSENRIGKGRKKRIKPIDVLNDKVAKFRNATNHKYANYIVQQCLKY |
| | NCGTIQLEDLQGISKEQTFLKNWTYFDLQEKIKNQANQYGIKVVKIDPSYTSQRCSECGYIHKKN |
| | RQDQSTFECQQCSFKIHADYNAAKNISVYNIEKVIQKQLELQEKLNLTKYKEQYIEQMENIN |
| 131 | MNKVVRLALICEHSDKDGNPVDYSDVYKLLWQLQAQTREIKNKTIQYCWEYSNFSSDYYKENHEY |
| | PKEKDVLHYALDGFVNDKFKVGNDLYSSNCSYMTRKVCAEFKKSKSDFLKGTRSIISYKSNQPLD |
| | LHNKSIRIEYKDNDFFAFLKLLKRPAFNRLGYKNSEIGFKVIVRDKSTRTILERCVDQIYGISAS |
| | KLIYNKKKKQWFLNLVYAFEPDNANNLDPSKILGVDLGIHYPICASVYGDLQRFTIHGGEIEEFR |
| | RRVESRKLSLIKQGKNCGDGRIGHGGKTRNKPVYSIEDRIARFRDTVNHKYSRALIDYAVKKECG |
| | TIQMEDLSGITAESDRFLKNWSYYDLQSKIEYKAKEKGIKIVYIDPKYSSQRCSKCGHIDKENRK |
| | TQSSFVCLKCGFEENADYNASQNIGIKDIDKIIENDLSSKCETDVN |
| 132 | MGKGVLAKVMKYELRYLDGCGDFSNMQEQVWALQRQTREILNRSIQIAFQWDCANSEHHRKTGEY |
| | LDLKTETGYKRLDGHIYNCLKGQYEDMATSNLNATIQKAWKKYNSSKKEILRGSMSIPSYKMNQP |
| | LTLDKNTVKLSEGERNPIVTLTLFSDKFKRAQGVSNVKFSMPLHDGTQRAIFANLMNGTYQLGEC |
| | QLVYKRPKWFLFVTYKFPPVEHPLDPDKILGVDMGEACALYASTFGEHGYLKIDGGEITKYAKKM |
| | EARIRSMQKQAAHCGEGRIGHGTKTRVSVVYQAKDKVARFRDTINHRYSKALIDYALKNQCGTIQ |
| | MEDLTGIKEDTGFPKFLRHWTYYDLQSKIEAKAAEHGIQVVKINPRHTSQRCSRCGHIDKANRTS |
| | QADFCCTKCGFSANADFNASQNISIRNIDKIIAKAIGANRKQT |
| 133 | MRIKIIAKKKGINMNKIMKYQILKPTNISWEDFGNILYNLRSEVRKIKNRTIALYHEWTNYTLEC |
| | HDRTGEWPKPKEVYNYGTMGGYIYDRLKGEVKYSNSVNFNSSVRDAMSKYDTHKKDILAGKASVP |
| | SMGDGQPIDIYNKNIVLHHLDNEKKDYAATLSLLNNGAKAELGLPSGRVDVILTIKNETQTAILD |
| | RCLSGEYRICGSQLIYEAAGKEKKGKKDKPKVWLYLCYGFEPEAPELDDSRIMGIDLGMKLPAVM |
| | AFNENDKKYEVIDDNRILDRKIRLDKMLSMSKHQCQWRCDGNSGHGRKKKVGVYENYANKSHNMS |
| | MTINHQWSKYIVDTAVKNKCGVIQMEDLSGIKASRQNFLGNWTYYDLQQKITYKAEEKGIKVIKV |
| | DPQYTSQMCPICGYINKRNRATQADFECLECGHIANADYNAARNIATPDIANIKNRLTQQKKEGK |
| | SID |
| 134 | MNENMCALTKIMKYELRYLDGFPDFSAMQNAVWPLQRQTREILNRTIQEAYHWDYFSATKKKETG |
| | EYPDLLKETGYKRLDGYIYHVLAPDYPDFSSSGVNATIQKAWKKYKSSRADVWKGEMSLPSYKSD |
| | QPIVLHAKQIKLSGDNRAAAVTLSLFSNKFKKEHAISGNVQFAITLHDNTQRTIYQKLRNGEYKL |
| | SESQLVYDKKKWFLYLAYSFNPAEHALDPEKILGVDMGEKFALYASSFGEYGHFKIEGSEVTEYA |
| | KALERRRRSLQQQARYCGEGRIGHGTKTRVGAVYREEDRIANFRSTINHRYSKALIEYAVKNGYG |
| | TIQMENLTGIKENLQFPRRLQHWTYYDLQSKIEAKAKEHGIAVVKVNPKHTSQRCSRCGHIAAEN |
| | RPKQEVFQCVKCGYACNADFNASQNISIKDIEKLIQETIGANPK |
| 135 | MQQVVRFELIKPIDNDWKVLGKVLRDLQYESRQVLNKTIQYNWEFSNCAIGFKEKFGINPKISDI |
| | SKYKGSHALQNYIYNELKNVYTKISTSNLTCLIEKATSQWKTFQKDVYKGERNPPEYKKSNTPII |
| | IHKQCINILKENGKYYADIALVSKKHLEEYKLNSCRFTLLLNTKNNGNKAILDMILNGELDYSQS |
| | QIIEKNKKWFLYLSYKVAEKVLPDDYNRIMGIDLGVNKAVVIAIHGTEIRDYIPGGEIIAYKNKM |
| | YNMVWHRQKQARYCGEGRIGHGRKTRLKNIYKIKEKIANFSDLTNHRYSKYIVELAKKHKCGVIQ |
| | MEDLSGLSTNNKFLKKYPIYDLQQKIIYKAEREGIKVVKIKPNYTSQMCSNCHFISEENRPKDER |
| | GWEYFKCVNCGLEIDADLNAARNMANSQIENIIKEQLKIQGIKNKNNGTDEESRKAVNS |
| 136 | MKYELTYLDGCGDFHNMQNELWALQRQTRELLNRTVQIAYHWDYKGSEHYRETGQPMDVYTETGY |
| | KRLDGYIYSCLKDDADSFSAANVNATIQKAWKKYKSSKSDIMQGKMSLPSYKRDQPLILYARNVK |
| | ISNEQRNPAVQFTLFSKSYKEEKGYSDVQFVMHLNDATQKAIFQKIASKEYGLGECQLVYSKPKW |
| | FLLLTYNFTPEDRRLDPDRILGVDMGETFALYASSKDEYGSLKIEGGEVREFAKRMEARKRSMQR |
| | QSAHCGEGRIGHGTKTRVSDVYKAEDKIANFRSTVNHRYSKKLIEYAVQHQYGTIQMEDLSGIRE |
| | STGFPKFLRHWTFYDLQQKIEAKAREQGIRVVKVDPSYTSQRCSKCGNIDKANRPTQAQFCCSKC |
| | GYKTNADFNASQNLSIRGIDRIIKETLGANPK |
| 137 | MSIKAIRLEIVKPYNESDAENVVTWNELGEALREVRYACSKAQNYVITERYLWERFKIDYKNQNG |
| | VYPDPKAFKERTNLYSQLTKMFPHVASNIINQTDRLATKKWSNEKKDVLSLKRSLTSFKLDVPIP |
| | VHYEGYKIFKVNDSEREKYIIRVTLLSKKSDKQMAYNLLLKVKDNSSKTILDRLISEELDSKIIQ |
| | IVSTNKKKWFCIIPYDFTERNTEVVDGRIMGIDLGIAKAVYYAFNDSYKRGSIDGGEIERFRKSV |
| | RARRITIQNQGKYCGDGRIGHGVKRRLKPVEILREKEKNERNLINHRYSRHLVEIAVKNKCAVIQ |
| | MEDLTGITKDNAFLKDWPYYDLQQKIKEKAAEYGILFKTINPYKTSQRCSRCGYIDRENRPEQSV |
| | FVCQNCGYGSLYLCENCNKEQNHAGICDTCGGKTNLITVNADYNAAKNIATENIEEIIKKEMGKE |
| | YNPPK |
| 138 | MSVKIASFSLIYKEDYNELNYDDLYNVLIQLQNESAKIANRAVQIYWENSNYCRQVKSMTGRFPT |
| | KDELLKHYGCSEQNYVYRLLTKEFYKNSTGNISTIIQFVGKRYKRYYPDYLSGKRSIESYKSSFP |
| | IYLCKNNIRVFKEDGKYYIKLGLISALYKSELSIHSGSVIFELGFGKSSSYKTLLDNIINQLFSL |
| | TSSKIIFLKKKIIIQLGYNTNSNIILDNSSCRVMGIDIGVAKPFVYAFNDITDFNFVDGNEIKNF |
| | QKQMLSRRQSLGRQTKNCADSKIGHGIHKRIEGIEKLGQKESNFRNRINHQYSRMIVDAAIKYKC |
| | TTIQIEDLSGISSENKFLKSWPYYDLQSKIEYKAKECGIDVVKINPKFTSQRCSKCGYISKENRK |
| | TQAGFKCKNCGFEENADLNAARNIAIPKIDSIIQESLRATT |
| 139 | MVKTTKIYLCMDSKDTYKLLWKLQDNTRMLKNKAVQMLWEWNNFSQEYKKEHEEYPKPKDILNYT |
| | VGGYMYDKLKSESLLASSNLSSTLQLVEKQFKTNAKDFLRGDKSIICFKKDQPLDIHNKSIRLSH |
| | ENGTFYADIVLMNKATANEHNGGGCALPFRLWIKDKSTRTIVERCYDGVYSVSGSKIKYDEKKKM |
| | WYINLSYGEDKYSTAELDKEQVLGVYLAQETPFTASVIGDHDRLQVSVNEIEHYRNAVESRRRSI |
| | LKQSAVCGEGRIGHGYKKRVEPMEKLSHKVADTRDTINHKYSKAIVEYAVKKGCGTIRMEKLTGI |
| | SETDRYLRNWPYFDLQTKIEYKAKERGIDIVYVDSADIQRCCRCGHVNEEETTGSRFKCTECGFE |
| | HDVAYNASQNLSAGGKDIKVNGRK |
| 140 | MRLYSIKCNKEKKLNEVKRMESEKEKYVVQSYGIKLIKPVGVDEGDWDFAGKVLRDLDYACFRVK |
| | NKAATRTYMNVIEKLEYENIHGKGTYDKYFKTRYGKTFSAYNSECAREDKELNKGDLYREYFNLM |
| | AREGEKVVKYNMKKILNGNASNITFERNQPVPITSRMIFITKESGKYYAELTLLSPEKAKELGRK |
| | GKNGTRIKFLLSSKGQEKVILDRLTSGEYDLRDSHIHVKKKGNKLTNYLIIAYRHKVKDDNDLIP |
| | NKVLGVDLGVSKAAYMAVSESPVSEYINGGEIEQFRNGVEARRNGMRNQLKYHSSNRSGHGRATK |
| | LKPLEKLREKVSNFRKLTNHRYAKFIVDTALKNNCSIIQMEELKGISKNDTFLKRWSYFDLQEKI |
| | ENKAIAVGIEVKKVSPKFTSQRCNKCGYIDKESRKSQEKFECVNCGHKTNADLNAARNLSMLNVE |
| | KEIKAQCKAQKIKY |
| 141 | MDKTLRDIQYVTCKASNKAMQMYYMWEYEKLEYKNKYGEYPNEKEMYGKTYRNVVEAEVKAIMNT |
| | INTSNTGQTNAFVMKKWNTDKKDIMNYRKSVASYKLNMPIYLKNSSYKILQGESGYEIDCAIFNK |
| | SQGLKHLTFTIDKLDGTKKATLNKLIESKNLSELINGAYKQGAMQIVKKKNKWCMLISFGFEAVE |
| | RELDTNRIVGVDVGIVNALTFQVWDNTTQKWDRLSWRDCLLDGKELIHYRQKIMARRIALLKNSK |
| | LSDENKGKAGHGRVKRIGPINTISDKVKAFRDTLNHKYSKYVIDFAIKNNCGCIQMEDLSGYSES |
| | VSETFLKNWSYFDLQSKIKYKAEENGLAINFIKPYHTSLRCSLCGNIQKENRDCRNNQSRFKCTV |
| | CGYEENADINAAKNISLPNIEQLIKEQLKIK |
| 142 | MEGEKEKYVVQSYGIKIIKPVGVDEGDWDFAGKVLRDLDYICFRVKNKAATRTYMNVIEKLEYEN |
| | VHGKGTYDKYFKTRYGKTFSAYNMEQAKEDKELNNGSFLREHFDSMAREGEKVVKNNMKKILNGS |
| | ASNITFKRNQPIPIRSRMIFITKESGKYYAELTLLSPEKAKELDRKGRKGTRIKFLLSSKGQEKV |
| | ILDRLTSGEYDLRDSHIHVKKKGNKLNNYLIVAYRLKVKDDKDLIPNKVLGVDLGISKAAYMAVS |
| | DSPVSEYINGGEIEQFRNGIEARRNSMRNQLKYHSSNRSGHGRSTKLIPLEKLRAKVNNFKELTN |
| | HRYAKFIVDTALKNRCTIIQMEDLSGISKTDTFLKRWSYSNLQEKIENKAKTKGIEVKKVSPKFT |
| | SQRCNKCGYIDKESRKSQEKFECVNCGHKTNADLNAARNLSMLDIEKVIKAQCKAQKIKH |
| 143 | MEGEKEKYVVQSYGIKLIKPVGVDEGDWDFAGKVLRDLDYICFRVKNKAATRTYMNVIEKLEYEN |
| | VHGKGTYDKYFKTRYGKTFSAYNMEQAKEDKELNNGSFLREHFDSMAREGEKVVKNNMKKILNGS |
| | ASNITFKRNQPIPIRSRMIFITKESGKYYAELTLLSPEKAKELDRKGRKGTRIKFLLSSKGQEKV |
| | ILDRLTSGEYDLRDSHIHVKKRGSKLNNYLIVAYRLKVKDDKDLIPNKILGVDLGISKAAYMAVS |
| | DSPVSEYINGGEIEQFRNGIEARRNSMRNQLKYHSSNRSGHGRSTKLIPLEKLRAKVNNFKELTN |
| | HRYAKFIVDTALKNRCTIIQMEDLSGISKTDTFLKRWSYSNLQEKIENKAKTKGIEVKKVSPKFT |
| | SQRCNKCGYIDKESRKSQEKFECVNCGHKTNADLNAARNLSMLDIEKVIKAQCKAQKIKH |
| 144 | MEGEKEKYVVQSYGIKLIKPVGVDEGDWDFAGKVLRDLDYICFRVKNKAATRTYMNVIEKLEYEN |
| | VHGKGTYDKYFKTRYGKTFSAYNMEQAKEDKELNNGSFLREHFDSMAREGEKVVKNNMKKILNGS |
| | ASNITFKRNQPIPIRSRMIFITKESGKYYAELTLLSPEKAKELDRKGRKGTRIKFLLSSKGQEKV |
| | ILDRLTSGEYDLRDSHIHVKKRGSKLNNYLIVAYRLKVKDDKDLIPNKVLGVDLGISKAAYMAVS |
| | DSPVSEYINGGEIEQFRNGIEARRNSMRNQLKYHSSNRSGHGRSTKLIPLEKLRAKVNNFKELTN |
| | HRYAKFIVDTALKNRCTHIQMEDLSGISKTDTFLKRWSYSNLQEKIENKAKTKGIEVKKVSPKFT |
| | SQRCNKCGYIDKESRKSQEKFECMNCGHKTNADLNAARNLSMLDIEKVIKAQCKAQKIKH |
| 145 | MEGEKEKYVVQSYGIKLIKPVGVDEGDWDFAGKVLRDLDYICFRVKNKAATRTYMNVIEKLEYEN |
| | VHGKGTYDKYFKTRYGKTFSAYNMEQAKEDKELNNGSFLREHFDSMAREGEKVVKNNMKKILNGS |
| | ASNITFERNQPIPIRSRMIFITKESGKYYAELTLLSPEKAKELDRKGRKGTRIKFLLSSKGQEKV |
| | ILDRLTSGEYDLRDSHIHVKKRGSKLNNYLIVAYRLKVKDDKDLIPNKVLGVDLGISKAAYMAVS |
| | DSPVSEYINGGEIEQFRNGIEARRNSMRNQLKYHSSNRSGHGRSTKLIPLEKLRAKVNNFKELTN |
| | HRYAKFIVDTALKNRCTIIQMEDLSGISKTDTFLKRWSYSNLQEKIENKAKTKGIEVKKVSPKFT |
| | SQRCNKCGYIDKESRKSQEKFECVNCGHKTNADLNAARNLSMLDIEKVIKAQCKAQKIKH |
| 146 | MEGEKEKYVVQSYGIKLIKPVGVDEGDWDFAGKVLRDLDYICFRVKNKAATRTYMNVIEKLEYEN |
| | VHGKGTYDKYFKTRYGKTFSAYNMEQAKEDKELNNGSFLREHFDSMAREGEKVEKNNMKKILNGS |
| | ASNITFKRNQPIPIRSRMIFITKESGKYYAELTLLSPEKAKELDRKGRKGTRIKFLLSSKGQEKV |
| | ILDRLTSGEYDLRDSHIHVKKKGNKLNNYLIVAYRLKVKDDKDLIPNKVLGVDLGISKTAYMAVS |
| | DSPVSEYINGGEIEQFRNGIEARRNSMRNQLKYHSSNRSGHGRSTKLIPLEKLRAKVNNFKELTN |
| | HRYAKFIVDTALKNRCSIIQMEDLSGISKTDTFLKRWSYSNLQEKIENKAKTKGIEVKKVSPKFT |
| | SQRCNKCGYIDKESRKSQEKFECVNCGHKTNADLNAARNLSMLDIEKVIKAQCKAQKIKH |
| 147 | MEGEKEKYVVQSYGIKLIKPVGVDEGDWDFAGKVLRDLDYICFRVKNKAATRTYMNVIEKLEYEN |
| | VHGKGTYDKYFKTRYGKTFSAYNMEQAKEDKELNNGSFLREHFDSMAREGEKVVKNNMKKILNGS |
| | ASNITFERNQPIPIRSRMIFITKESGKYYAELTLLSPEKAKELDRKGRKGTRIKFLLSSKGQEKV |
| | ILDRLTSGEYDLRDSHIHVKKKGNKLNNYLIVAYRLKVKDDKDLIPNKVLGVDLGISKTAYMAVS |
| | DSPVSEYINGGEIEQFRNGIEARRNSMRNQLKYHSSNRSGHGRSTKLIPLEKLRAKVNNFKELTN |
| | HRYAKFIVDTALKNRCSIIQMEDLSGISKTDTFLKRWSYSNLQEKIENKAKTKGIEVKKVSPKFT |
| | SQRCNKCGYIDKESRKSQEKFECVNCGHKTNADLNAARNLSMLDIEKVIKTQCKAQKIKH |
| 148 | MEGEKEKYVVQSYGIKLIKPVGVDEGDWDFAGKVLRDLDYICFRVKNKAATRTYMNVIEKLEYEN |
| | VHGKGTYDKYFKTRYGKTFSAYNMEQAKEDKELNNGSFLREHFDSMAREGEKVVKNNMKKILNGS |
| | ASNITFKRNQPIPIRSRMIFITKESGKYYAELTLLSPEKAKELDRKGRKGTRIKFLLSSKGQEKV |
| | ILDRLTSGEYDLRDSHIHVKKKGNKLNNYLIVAYRLKVKDDKDLIPNKVLGVDLGISKTAYMAVS |
| | DSPVSEYINGGEIEQFRNGIEARRNSMRNQLKYHSSNRSGHGRSTKLIPLEKLRAKVNNFKELTN |
| | HRYAKFIVDTALKNRCSIIQMEDLSGISKTDTFLKRWSYSNLQEKIENKAKTKGIEVKKVSPKFT |
| | SQRCNKCGYIDKESRKSQEKFECVNCGHKTNADLNAARNLSMLDIEKVIKAQCKAQKIKH |
| 149 | MEGEKEKYVVQSYGIKLIKPVGVDEGDWDFAGKVLRDLDYICFRVKNKAATRTYMNVIEKLEYEN |
| | VHGKGTYDKYFKTRYGKTFSAYNMEQAKEDKELNNGSFLREHFDSMAREGEKVVKNNMKKILNGS |
| | ASNITFERNQPIPIRSRMIFITKESGKYYAELTLLSPEKAKELDRKGRKGTRIKFLLSSKGQEKV |
| | ILDRLTSGEYDLRDSHIHVKKKGNKLNNYLIVAYRLKVKDDKDLIPNKVLGVDLGISKAAYMAVS |
| | DSPVSEYINGGEIEQFRNGIEARRNSMRNQLKYHSSNRSGHGRSTKLIPLEKLRAKVNNFKELTN |
| | HRYANFIVDTALKNRCTIIQMEDLSGISKTDTFLKRWSYSNLQEKIENKAKTKGIEVKKVSPKFT |
| | SQRCNKCGYIDKESRKSQEKFECVNCGHKTNADLNAARNLSMLDIEKVIKAQCKAQKIKH |
| 150 | MEGEKEKYVVQSYGIKLIKPVGVDEGDWDFAGKVLRDLDYICFRVKNKTATRTYMNVIEKLEYEN |
| | VHGKGTYDKYFKTRYGKTFSAYNMEQAKEDKELNNGSFLREHFDSMAREGEKVVKNNMKKILNGS |
| | ASNITFERNQPIPIRSRMIFITKESGKYYAELTLLSPEKAKELDRKGRKGTRIKFLLSSKGQEKV |
| | ILDRLTSGEYDLRDSHIHVKKRGSKLNNYLIVAYRLKVKDDKDLIPNKVLGVDLGISKAAYMAVS |
| | DSPVSEYINGGEIEQFRNGIEARRNSMRNQLKYHSSNRSGHGRSTKLIPLEKLRAKVNNFKELTN |
| | HRYAKFIVDTALKNRCTHIQMEDLSGISKTDTFLKRWSYSNLQEKIENKAKTKGIEVKKVSPKFT |
| | SQRCNKCGYIDKESRKSQEKFECVNCGHKTNADLNAARNLSMLDIEKVIKAQCKAQKIKH |
| 151 | MATKCIKLAGEYVKENSLEKDKFFKELRDIQYKTWLACNRAITYYYSNDMQNFIQKDVGIPKEDD |
| | KLLYGKAFKSWVANRISEILGDGISSYATDCISQFVSNRYKNDKKAGLLKGNVALSQFERDIPVM |
| | LRERAYSHIDTPKGLGIEISFFSKTKQQELGIKRILFTFPKIDGSSKSILTRIMDKTYKQGSIQI |
| | TYNKRKKKWMFAISYTFENKLEKVLNDNLVMGIDLGITKVATMSIYDIEKHQYKNMCFKEQTIDG |
| | TELIHYRQKIEARRKSLSISSKWASDNATGHGYKRRMKKANNIGDKYNRFKDTYNHKVSRYIVDL |
| | AYKHGVKTIQMEDLSGFSEHQSESLLKNWSYYDLQNKIKYKAEEKGINTIFINPQYTSKRCSKCG |
| | NIHEENRDCKNNQAKFECIICGHKENADINASKNIAIPYIDKIIKEYIKDTI |
| 152 | MATKCIKLAGEYAKENSLEKDKFFKELRDIQYKTWLACNRAITYYYSNDMQNFIQKDVGIPKEDD |
| | KLLYGKAFKSWVANRISEILGDGISSYATDCISQFVSNRYKNDKKAGLLKGNVALSQFKRDIPVM |
| | LRERAYSHIDTPKGLGIEISFFSKTKQQELGIKRILFTFPKIDGSSKSILTRIMDKTYKQGSIQI |
| | TYNKRKKKWMFAISYTFENKLEKVLNDNLVMGIDLGITKVATMSIYDIEKHQYKNMCFKEQTIDG |
| | TELIHYRQKIEARRKSLSISSKWASDNATGHGYKRRMKKANNIGDKYNRFKDTYNHKVSRYIVDL |
| | AYKHGVKTIQMEDLSGFSEHQSESLLKNWSYYDLQNKIKYKAEEKGINTIFINPQYTSKRCSKCG |
| | NIHEENRDCKNNQAKFECIICGHKENADINASKNIAIPYIDKIIKEYIKDTI |
| 153 | MATKCIKLAGEYAKENSLEKDKFFKELRDIQYKTWLACNRAITYFYSNDMQNLIQKDVGIPKEDD |
| | KTIFGKSFVAWVENRMNEIMEGISSANVAQTRQFVNNRYSQDKKNGLLKGSVSLSQFKRDLPIII |
| | HNKAYNVIETSKGLGVEISFFNKEKQKELNVKRIKLLFPKLDNSSKQILIRLMDKTYKQGSIQVT |
| | YNRRKKKWMFAISYTFENKLEKVLDDNLVMGIDLGITKVATMSIYDIEKHEYKKMYFKEQTIDGT |
| | ELIHYRQRIEARRKSLSIASKWASDNATGHGYKRRMKKANNIGDKYNRFKDTYNHKVSRYIVDLA |
| | YKHGVKTIQMEDLSGFSEHQSESLLKNWSYYDLQNKIKYKAEEKGINTIFINPQYTSKRCSKCGN |
| | IHEENRDCKNNQSKFECVVCGHKENADINASKNIAIPYIDKIIKEYIKDTK |
| 154 | MATKCIKLAGEYAKENSLEKDKFFKELRDIQYKTWLACNRAITYFYSNDMQNLIQKDVGIPKEDD |
| | KLLYGKAFKSWVANRISEILGDGISSYATDCITQFVSNRYKNDKKAGLLKGNVALSQFKRDIPVM |
| | LRERAYSIIDTPKGLGVEISFFSKTKQQELGIKRILFTFPKIDGSSKSILTRIMDKTYKQGSIQI |
| | TYNKRKKKWMFAISYTFENKLEKVLDDNLVMGIDLGITKVATMSIYDIEKHEYKKMYFKNQTIDG |
| | TELIHYRQRIEARRKSLSIASKWASDNATGHGYKRRMKKVNNIGDKYNRFKDTYNHKVSRYIVDL |
| | AYKYGVKTIQMEELRGFSEHQSVSLLKNWSYYDLQNKIKYKAEEKGINTIFINPQYTSKRCSKCG |
| | NIHEENRDCKNNQAKFECVICGHKENADINASKNIAIPYIDEIIKEYIKDTI |
| 155 | MKINKCIKVTLIKCLNYDYKEIKQIIRDFNYTACKASNKAMRMWLFHTQDMIDKRNEDKLFNQIQ |
| | YEKDTYGKSYRNVIEGEMKKLMPLANTSNVGTLHQQLVQNDWGRLKKDILSCKANIPNYKINTPY |
| | FIKNDNFKLRNHNGYFVGIAFFNKEGLQQYGYKIGHKFEFQIDKLDGNKKATINKIINGEYKQGS |
| | AQISISKKGKIELIISYGFEKEEIPVLDNNRILGIDLGITNVATMSAYDSIKDEYDYFSWKTNVI |
| | SGKELIAFRQKYYNLRRDLSIASKTAGKGRCGHGYKTKMKPVDKIRNRIANFADTYNHKISKYIV |
| | EFAVKNRCGVIQMEDLSGATSEVHNKMLKDWSYYDLQQKIEYKAREQGIEVKKVNPKYTSKRCNK |
| | CGCIHEDNRDCKNNQAKFECKVCGHSENADINASRNIAIPEIDKIIDKTEILHSENRQAS |
| 156 | MKFNKCIKVTLIRCLNYDYRETKQIVRDFQYKYSKAYNMATNYLYLWDTNSMNLKNLYDTKIVDK |
| | ELLGKSKCAWIENRMNEIIQDSNTSNVAQARQDVVNKYNKCKKDGLFKGKVSLPTYKLDSKVIIH |
| | NNSYKLRNHNGYFIDIGLLNKDKQKELNVGRFEFQIDKLDGNKKATINKIINGEYKQGSAQISIS |
| | KKGKIELIISYSFDKEEVPVLDKNRVLGIDLGITNVATMSVYDSIKDEYDYFSWKINVISGKELI |
| | AFRQKYYNLRRDISIASKVAGKGRCGHGYKTKMKPVDKIRNKIANFSDTYNHKISKYIVEFAVKN |
| | NCGVIQMEDLSGTTADTNNKMLKDWSYYGLQQKIEYKAKEQGIEVKKVNPKYTSKRCNKCGCIHE |
| | DNRDCKNSQAKFECKVCGHKDNADINASKNIAIPDIDIIIKETEIL |
| 157 | MNSIKINKCIKVTLKKCLNLDIKEVKNIIKDMNYLACKASNKAIKMWQLHTEEMMERKFEDKNFN |
| | ISQYEKDTYKKSYRNVIEGKMKDIMDICNTSNVGTLHQQLVQNDWGRLKKDVLNYRANVPTYKLD |
| | TPYFIKNHNYKLYNNGGWFVDIAFFNKQGLIQYGYKVGHKFEFEIDKIDNNKKSTITKINGEYKQ |
| | GSAQISLSKKGKIELIISFSFEKELKNNLDKNRILGIDLGIVNTATMSIWDNNKQAWDWVDYKEN |
| | RIDGKELIKFRQKLFSMGMSNSEIEKEIFKANSKIKENQLRKRELGVIDGLELAKYRDTVYKKRR |
| | EMSIASKYAGKGRSGHGRKTKMKPVDKIRNKVYNFADTYNHKYSKYIVDFAVKHNCGIIQMEDLS |
| | NATANKKEKFLKDWSYFDLQTKIEYKAKEYGIEVIKINPKYTSKRCSKCGGIHIDNRDCQHNQAN |
| | FECKICGHKENADINASRNISIPYIDKIIAKTKVQAD |
| 158 | MVTKVVECKIIGFDSLLFEKEDVTENAKKDNKKAKEAWKRNIEMIDQAVRDFVAAKNRAITYLFT |
| | EYIKVRDAMATSGESSFSSVWKKMHDEKGRSKAMYHYLVKALPGYLTGNVSEGSRRLEKRFDQAV |
| | KDGLLYGRVSLPSFRDNAALDFRGDSVKIKQEIIKGEEKLYVELNLKRGIKLHCNIGFSGNKGAK |
| | AVAERIGKGEYKAGTASIVKRKSRYFIQICYSFDSDIYMRSNGMVGLAEGLDPKRTLGVDLGLAR |
| | PVAMQPYDSDSRKYLSGGKNDEFVNTERYTNRFTISGDRIAAFRKQVEKKKSVLQRDLRDVSPGH |
| | TGKGRIKRIKPVEKFKDRIANFRKTTNFVYAKRIVDTAIKYHCGTIRIEDLTIDVSSRKSERFLK |
| | TDWTYYDLQQKIESRAANFGIVVQKVAPQYTSQKCSKCGHVSADNRKDQSHFKCVSCGFEMNADL |
| | NAARNIAALSPNDKIKKEKNKEAKDGQMQLEFILN |
| 159 | MVTKVVKCKVVGFDSLPFEKKDAAGNAKINNKKAKEAWNRNIEMIDQAVKDFVAAKNRGITYLFT |
| | ESIKIQDAMDASGESSFYDAWKKMHGGTQLRTTMDHFLVKELPGYLAENVCESSGRLVTRFNQAK |
| | KDGLFKGCVSLPSFRDNAALDFRGSSVKIKQETINGEEKLYVELRFKRGIKLHCNIDFSGNNGAK |
| | AIAKRVCNGEYKSGTASIVKRKGKYFIYICYSFEPETYMRLNGMDGLAEKLDTKRILGVDLGLAK |
| | PVAMQPYDSISQKYLSGGKNDEFVNTEEYTNRFTISGDQITAFRKQVEKRKRALQRDLRDVSPGH |
| | TGKGRIKRIKPVETFKDRIANFRNTTNFIYAKRIVDTAIKYHCGIIRIEALTIDVSSRKSERFLK |
| | TDWTYYDLQQKIESRAANFGIVVQKVKPNYTSQKCSKCGHVSADNRKDQSHFKCVSCGFEMNADL |
| | NAARNIATLSPTDKINKKKKREVKDDQV |
| 160 | MTKTKMITKATRFQIIKPLDTGWDELGQVLRDLSYHTTKLCNMAVQLYWQHHNYRLAHKEETGKY |
| | PAAAEDKERYGCSFRNHVYRKMREMYPDMASSNTSQTNQFAMSRWQNDVREIMRLQKSIPSFRLG |
| | TPAQVANANYTLSVEEQDNGRSSFIATITLLSLAAGKQTRYSILLDGGDRSKKAIFRRIMEGEYK |
| | QGAMQITYNKRKKKWFCIVSFSFIPEKQNRELDPDRAMGLMFGTGNYTVFAAYSVGQKRYTLPAG |
| | EVIATEKKIHAIAERRKEIQRHAGYRGHGIKRKLQGTEVLAGQASAIRDTLNHKYSRRVVDVAMA |
| | NRCGTIKILDKLVTPGASVSELVNYPWAGVVEKIKYKAEEKGIAVKVVSLDGTRCHKCDAEIEID |
| | SDNESPLTCPSCGAKIDKDYNTAKRIAKS |
| 161 | MITNTCKLEVSFIDTKFSDLFAMINATTQVKRYATDRITEWKSFSSRYHEENGEYPKLEDIYGFK |
| | SIEGLIYHELSKKKTGLSSRSLIATIHSCFLSYKQASKKHSHPNWSDSQPIIMQGKYLNLHHESG |
| | NYIVDYPLESKQYRVEHETTHILRMRLNTKENHHKNILDRILSGEYHLCQSQLKYVKKQNKSHWY |
| | FLLAYSHKSEGNPNIDPTRVLGVFMSENVALYASSKRLPMVFKIDGGEIPTFASRQEAIIRSKQR |
| | QAACCGDGRIGHGYKTRTKSIYPNKQTLAHFRETINSRYAKALIDFAKTNGFGEVRLEDLKGIKA |
| | DREFPRFLIHWTYFDLQEKIKNAARKCGIKVTLVSSETIGITCSSCGHIDPNNRDRGVKNRFVCT |
| | KCNIVKQTDWNASQVLSSLEI |
| 162 | MLTKCIKIPIEYSKDNILKSEEFYKELRDIQYKSWRACNRALTYFYMHDMENIMLKESGKDIKSD |
| | KELYGKTYGSWIENRMNEIMKGVLSNNVAQTRQYISNRYGQDRKNGLLKGNVSLSEIKRNMPIII |
| | HAKAFKIIDIIKGLGVEVSFFNLNKQRELGVKKIKFLFPKIHSSEKSILKRLVDKSYKQGIAQLS |
| | FNERKKKWLLTISYSFEKRIEELDEDLVMGIDLGISKITTFSILNAKTKEYIQMNFKDKFVDGKE |
| | LIHYRQKLEARRRELSIASKYTSKNNLGHGYKTKMQSVNMAGDKYNRFKETYNHKVSRFIIEIAL |
| | RYRVKNIQMEDLSGFSEHQTESLLGNWSYYDLQNKIAYKAFELGINIIFIDPKYTSQRCNKCGNI |
| | NSKNRNCKENQEKFECIRCGYKENADINASMNIAIPNIENIINNTIKIK |
| 163 | MKEKYFVKVAKFEILKPANGMTWKEFRQLLMSVRYRVFRLANLCVSENFLQFHLWRKKEIDKIPT |
| | LKISELNRQLREMLLEEKKTSDAEQTRICKRGALPASIVDALSQYKIRALTAKSKWRDVIRGNAS |
| | LPSFRNDAAIPICCHKPSHRRLEKTSNGNVELELMICMKPYPRIILKTEKISGNMKATLERLLAN |
| | RGNSDTGYQQRFFEVVQDRQEGRRWYLHVTYKFPASLLRLNPQVIVGIDLGFSCPLYAAINNGLA |
| | RLGWRHYESLGKRIRNLQNQVFARRRSMQSGGNASLTMDTARGGHGRKRILRPIEKLAGRVNNAY |
| | STLNHQLSRSVIEFAKNHGAGVIQMENLEGLKEQLTGTFLGSRWRYHELQQFIEYKAKEVGIEVR |
| | KINPQYTSRRCSECGYINIKFDRAFRDANRKDGKTAKFICPKCKWEGDPDYNAARNLATLDIENL |
| | IRQQLINQGIPSEREETPVL |
| 164 | MQECKYVQRSIKLKIVCPLDENITWEEFGFLLRGLSFKVARASNFCMLHHLLYAMKLETVHLNRK |
| | GGLYCYPYLAEEYPEVPAGILCAAETRARKLFKQHAVEILRSDRALPNFRKDVGIPVPAASYKIM |
| | QDGKGDFLMEIQLLSRQAAKTGKLSGRICLALATNWRDKTAVAALQKIAEGSLKTGVGTLFREKK |
| | NWYFVVPYQREKGLSKEATENDECVMGVKLGVQNALVYAFDRSLKRGSLSGEEVKARQEQFYARK |
| | QNILEQYKWSGRKGHGRERALKPIQELYEKERNFRNTANLRYANWIVEIAVKNRCGEIHLESGKG |
| | VGFGQNSIMLHFWPVWDLKNRIKQKAEEHGIKVVECNVPDIWGACSECGAKSGNSVEKQSFSCPS |
| | CGYGKQEEKYGVGYVSAEYNAARNLALWRKEE |
| 165 | MGKVHTRTIKLKLIVSTEDKNVAWKRIRQISNDAWRAANWIASGQLFNDQLVRRIYARRKINPRE |
| | DSEAVEQIEKEFEAFFGTKRQATTEWDIKEAFPDLPPYVINPLNHVVVASYKKEKPDMLSGNRSL |
| | RTYKRGMPIQTAKAAINFSKNETGQHFVKWTLGRKEFIDFEIYYGQDRANNRLTIDRIINGQIDY |
| | SAPMIQLKDKNLFLLLPVKEPEIAADLDPDIALGVDLGVATPAYMALSKGPARRAVGDKEDFLKV |
| | RLQMQSRKRRLQRSIKSAHGGKGRQKKLKALNQIGEKERQFARTYNHYISREIVNFAIRHNAGTI |
| | KLEMLEGFGQEEQQAFVLRNWSYFELQTFIEYKAKKVGIKILKIDPYRTSQTCSSCGHFEEGQRK |
| | DQSTFECKNCGEKLHADYNAALNIARSKKIVTKKEQCEYYLNRNQNTI |
| 166 | MADQGKTLFKVMPLRILKPVGDTRWDALGQMLRDTRYRVYRLANLAISEAYLGFHLFRTGKAEQF |
| | KTDTIGQLSRRLRQMLLDEKVPRDNLDRSSMTGAVPDTVASGLHQYKIRSVTNVNKWQQVVRGKS |
| | SLPTFRADMAVPIRCDKAGQRRLERNTQGDVELDLMICRRPYPRVVLATGTLGPGQQTILDRLLE |
| | NEDNAKSGYRQRLFEVKEDSQTHQWWLYVTYEFPAPVLPVAHGDIVVGIDLGVSVPLYAAINNGH |
| | ARLGRRQFQALGYRIRTLQNQVIMRRRAIQRGGRVGVSQPTARSGHGVHRKLLPTEKLRRRIDKS |
| | YTTLNHQLSAAVIDFAKNHGAGVIQIEDLSTLKEQLVGTFLGGRWRYHQFQQFLAYKAKENGITL |
| | REVNPKYTSRRCSECGFIHVDFDRAFRDRHHTEGMVTKFVCPQCKYEADPDYNAARNIATLDIEQ |
| | RIKVQCQVQKLM |
| 167 | MAKKQDDNTMTIARKITLIPVASERKEWKKRIDAFLEKDFPMQIEIKKKQIKNTSKPERVDGYKQ |
| | QLAELEKQYEEFKENGIKEYTHKMASDYTYDIVRRAMESEARRKNYILSYIYTKMIQDEVANLPT |
| | LTEKNKWVSANVKECYRKAGNKNGSIFTNVDIDNPLAGYGSDFGQAFTRKIKKLIKDGILEGNVS |
| | VPNYKLDSPFALSNQNFGIFTDCENITELKKNIGKPTYPVYVCLGKHGLPTIAKFKINFGHKQNK |
| | NKAELISTIIKILTGEYSVGGSTFGIDDDKIEMNLSITMQKQKMDLDENTVVGVDLGLAVPAVCA |
| | LNNNEYDKQYIGSGNDLVTRRTKFQNEYTQLQKALKLAKGGHGRKRKLLALERLKEKEKNFVDTY |
| | CHRVSKKVVDYAIKHRAKYINIENLKGYDSSEFVLRNWSFYKLQQYITYKAEQCGIEVRKINPSF |
| | TSQVCSFCGHWEEGQRKDQATFKCKNPNCKSHKLYTVNADYNAARNIAMSTQFTDDKFKCSKKTI |
| | QDAADYYGIVLEDDKNNDNKKAA |
| 168 | MILLIQIEEVNQMITSRKIKLAIVSDNKDTAYSFIREETRNQNRALNVAYTHLYFEYVAQEKLKQ |
| | SDKEYQQHLEKYKNAAAKKYQEFLTIKEKSKSDENLQPKMDKVRETYNKAMEKVYKIEKDYSKKA |
| | REIYQQSVGLAKQTRLGKLIKSEFDLHYDTVDRIGSNAMSDFSNDRKSGILSGERSLRNYKKTNP |
| | LMVRARSMKLYEEDNNFYIKWINDIVFKIIISAGSKQRMNIAELKSVFIKLLSGQCKMCDSSISL |
| | DKGLILNLSIDMPITKENVFIPNRVLGVDLGLKIPAYVSLNDTHYIKGAIGNIDDFLKVRTGLQS |
| | QRRRLQKSLQSTGGGKGRRKKLQALERLKTKEKNFVNTYNHFLSKNIVQFAVKNNAGAIHMEELK |
| | FDKMKNKSLLRNWSYYQLQTMVEYKAKSEGIEVYYVDASYTSQTCSKCGNLEEGQREARDTFVCK |
| | KCGYNVHADYNASQNIAKSTKAINKTIEITNIV |
| 169 | MPKQEEEGMIITRKIEIACRNKDQYPLLQDYLKQCRLMANKAMTNYYILWQEMLETKPKNQTQKD |
| | YFLKKHGCSFQNTGYRYLRRCFGGMPSYLRRDVANRVYQDFRADLKNGLLKGERSLRNYRTGHLP |
| | LHNKDIQLCKEETSSDYLLRWRMGIEFVLLFGRDRSGNRIIADRLIEGTYRVSGSQIYKDWRKKK |
| | WFVLLCVKIPQQDNNLDETAVVGVDLGLSTPAVCALSNGMARAYIGSSNLLKRFEMQKKQRSRQR |
| | EFIPATNKHGRQKVVKAIYAMKDKERRFVHSFNHAISKKIIQFALKYKAAVIRMEDLSGYNGDET |
| | VLRNWSYYELQSMIEYKAEKEKIRVEKIPACYTSRACSQCGFIDKENRKTQAQFECVKCGFKENA |
| | DYNAALNIARGGVKIPEENQEDFGENE |
| 170 | MILTRKIQIIPLGEKEEIDRVYKYLRDGIFYQNKAMNQYMSALYIAAIKDISKEDRKELNRLYSR |
| | VSNSKKGSAYDKSIEFAKNMNLGYVVKQVKQDFANSCKNGLLCGKVSLPTYRKNNPLLVHVNFVR |
| | LRSTNYHQDNGMYHNYESHTDFLDHLYSKDLEVFIKFANNITFKMIFGNPHKSAYLRSEIQQIFE |
| | ENYKVCGSSIQIDGKKIILNLSMDIPKQELELDENIVVGVDLGLAIPAMCGLNINDYIRQSIGSK |
| | DDFLRIRTQLQSQRRRLQKSLASTSGGHGRQKKLKPLEKLKDRERNFVKTYNHYVSKNVVDFAVK |
| | NKAKYINVEDLSGFDSNQFILRNWSFYELQQFITYKAAKYGIEVRKINPYHTSQICSCCGHWEEG |
| | QRIDQAHFKCKSCGAELNADFNASRNIAMSTDFV |
| 171 | MQRSIILKILRPYDESIKWEELGYLLRGLSYKVCKISNYCMTHHLLRALNLETENLNPQGHLYCY |
| | PRLAQDYPDVPAGILCAAEGRARKVFLKCGKEVLRSETALPNFRKDCSIPIPVAGYSLLKAGEDT |
| | YVANVQLLSRQAAKTQKLPGRIQLVLAKNWRDRSAGPVLQQLTEGTLKRGIASLFRRKRDWYISI |
| | PYEAEAQKTEDGSFAPGLVMGVILGTQCALAYAFNRSPKRGAIGGEEILAHKEKFRARKKHIREQ |
| | YNWSGRKGHGRGSALKPLQALYEKERNYRNLTNERYAKWVIEIAKKNRCGKIHLDGGSAGGTGTP |
| | HVMLAYWPQGELRKKIRYKAEACGIEVVECDGRDVWNRCSKCGAVQEVSGERRWFTCSHCGYGKD |
| | DKKTSSSFVTVDYNAARNMAIMESKP |
| 172 | MATEYTCITRKIEVHLHKHGDTEEAAQRLKDEYHIWDNINDNLYKAANRIVSHCFENDAYEYRLK |
| | IHSPEFRQIEKSLQHAKKNKLSEDDIKKLKAERKSLCADFREQRLAFLRGGATEGANPEQNSTYQ |
| | VVSHEFLDVIPSDILTCLNQNIASVYKQYALEVEMGKRTIPNFKKGIPVPFAIKQNKQLRLRKRN |
| | DGSVYVLLPRELEWDLSFGRDRSNNREIVERVLSGQYDVGNSSIQETRSGKRFLLLVVKIPKASR |
| | TLDTKKVVGVDLGIATPLYAALNDNEYGGLSIGSQEQFLKIRMRMTAQKRELQRNLRYTTNGGHG |
| | RTQKLQALERLEGKERNWVHLQNHIFSKSIIEYALKNNAGVIQMERLTGFGHDKNDEVGTEYKFI |
| | LRYWSFFELQSMIEYKAKAAGIEVRYINPYHTSQTCSFCGKYEKGQRINQATFVCKNPDCVKGKG |
| | RQKSDGTYEGINADWNAARNIALSEEFVDKKKK |
| 173 | MNTVRKIKLTILGDIETRNKQYKWIRDEQYKQYRALNLCMTYMVTNLMLRNSESGLENRKEKEIL |
| | KIQNKIKKDEEKLEKELKKEKSKEEKIQDIKFNIEELKLQKEKLENELRNIKEYRSNIDEEFKKM |
| | YVDDLYNVLNKVPFQHEDMKSLVTQRIKKDFNNDVKEIMRGDRSVRNYKRNFPILTRGRDLKFQY |
| | IEKSEDIEIRWIEGIKFKCILGKTSKSLELKHTLHKVINKEYKVCDSSLQFDKNNNLILNLILDI |
| | PQDNKYEKITNRVVGVDLGLKIPAYVALNDTKYIRKAIGSIDDFLKVRTQMQSRVRKLQKSLQTA |
| | RGGKGRNKKMKALDRFREKERNFARNYNHFLSYNIVKFALDNKAEQINLELLEMKETQNKSILRN |
| | WSYYQLQSFIKYKAERIGIKVEYIDPYHTSQICSECGNYEEGQRVEQATFVCKRCGHKINADYNA |
| | ARNIAMSKEYISKKEESKYYKNNKNMV |
| 174 | MSKNTYTITRKIQLIPVGDKEEVNRVYTYLRDGMKAQNMALNQYISALYFAMQNDATKDSRKELR |
| | NLFSRISTSKKGSAYDDTIEFAKGLPSCMMTRKVESDFKNAMKKGLKYGKLSLPTYRDTNPLLIH |
| | IDYVRLRSTNPHLDNGLYHNYKNHTEFLEHLYDENLELFIKFANKITFKIILGNPHKSSELRSVF |
| | KNIFEDCYHIQGSSIEIEKNKIILNMSMSIPVKKIELDENIVVGVDLGMAIPAVCALNTNEYIHK |
| | SLGNYNDFIRERTKIQAQKKRMQKSLTYTNGGHGRKKKLKPLNRFKERERNWVKTYNHKISKQII |
| | DFAIKNKAKYINLEDLSGIAKEDKDKFVLRNWSYFELQQFITYKAEKYGIIVRKIKPEYTSQKCS |
| | CCGHLEENQRINQSEFICKNPNCKNFGKIVNADYNGARNIAMSTNFVEEKETKSKPKKVA |
| 175 | MQRIIILKILRPYDESIKWEDLGYLLHGLSYKVCKISNYCMTHHLLRALNLETENLNPQGHLYCY |
| | PRLAQDYPDVPAGILCTAEGRARKVFQKCGKEVLRSETVLPNFRKDCSIPIPVAGYSLLKAGEDT |
| | YLANIQFLSRQAAKTQKLPGRIQLVLANNWRDRSAGKVLQQLTEGTLKRGIASLFRRKRDWYISI |
| | PYEVEAQKTEDGSFAPGLVMGVILGTQCALAYAFNRSPKRGAIGGEEVLAHKEKFRARKKHIREQ |
| | YNWSGRKGHGRGSALKPLQALYEKERNYRNLTNERYAKWVIEIAKKNRCGKIHLDGGSAGGTGTP |
| | HVMLAYWPQGELRKKIRYKAEACGIEVVECDGRDVWNRCSKCGAVQEVSGERRWFTCSHCGYGKD |
| | DKKTSSSFVTVDYNAARNMAIMESKP |
| 176 | MATITRKIELYIDKSNLTDDEYKAQWQYIRQIDNTLFLAANRISSHCLLNDELEIRLKLQMPEYC |
| | EIEKSLRNSKKNKLSKEEISELKLRRKELDAVVKRQKEEFLKTSQNSTYQLVAYEFTNIPTEILT |
| | NLNNDIVGKYGKARLDIIKGIKSPSTYKKGIPIPFSVNKKSPFVFIDGDLEWFKGKTKETTGKIL |
| | RFKLHFGKDKSNNRAIVERLVESAKLGKKKGEAYIVNNSSIQLVEKENTTKIFLLLSLDIPTTKR |
| | ALDSNLVMGIDLGINYPIYYATNGNAYIKGHIGDRDSFLNERMVFQRRFRELQRLQCTQGGRGRL |
| | KKLAPLEKLREKERNWVRTKNHIFSREIIKCALKIGAGTIHLEKLKNIGKDKDGNIEDSKKYILR |
| | NWSYHELQEMIEYKAAMEGITVKYVNPAYTSQTCSCCGNRGERISQSVFKCLCPECSEYGKEVNA |
| | DYNAARNIAKSEIFVKK |
| 177 | MDNTITITRKYTLIPTFSDTKEWTKKVMEYTKTSYIEKIKYYEEKIKKTKKKDKEEREKYENRLS |
| | QLKEQQLDFEENGTLLQTNVNDYTYDLVREAMASESDRKNMIISYVCGELINRDAKDMDFKERNK |
| | LISELCNYGYRVKGSKKGSLFDHLDIDNPLGGYGVSFCQDLTKKIKELVNNKRWLDGKVSTLHYK |
| | DDSPFSIAKATMGFAHDYDSFEELCEHIREKDCNLYFNFGNNGKPTIARFKINLGANRKNKDELI |
| | STIIRVYSGEYQYCGSSIGIEGTKIILNLSMKIPKQEKELDENTVVGVDLGIAVPAVCALNNNVY |
| | ARKFVGNKDDFFKVRKQLNAQYKRVQSALKRASGGHGRKKKLKALERLRKKEAHFVETYCHMVSK |
| | AVVDFALKYNAKYINLENLTGYDTDDIVLRNWSYYKLQQYITYKASKYGIEVRKINPCYTSQICS |
| | ECGNYHPENRPKGDKGQAYFNCHNKECITHGKKSPYQYGINADFNAARNIAKSTLWMGKGKVTEE |
| | SKKKAREYYGIEEEYEELNKEVA |
| 178 | MITARKIKLTIAENREEGYSFIRKELQEQNKALNMAINHLYFNYVAREKIKLADETYKVKLEEGE |
| | CYLERKYIELKEAKTDKQKENIKKSIEATKKKLETLRKVENKEVSNNFKEIIATSEQINLRDLIS |
| | NNFNLKSDTKDKLTQKVVQDFYNDIVGVLRGERTLRRYKKDNPLYIRGRSLTLYREGEDYYIKWM |
| | NGIVFKCVLGVKKQNSLELQKTLDKVIFEEYKLCDSSIGFKDNKLILNLTLDIPVSNTNKFEKVI |
| | GRIVGVDLGMKIPAYCALNDSEDVRKAIGSIDDFLKVRTQMRSRRRKLQRALKSTNGGQGKNKKL |
| | SALNSFEAKEKNFAKTYNHFLSSNIIKFATDNKAEQINMEFLSLSETQNKSVLSNWSYYQLQQMI |
| | EYKAERIGIKVKYVDPYLTSQTCSECGHYEDGQREVQSEFQCKKCGCKTNADYNASRNIAKSDKY |
| | ITKKEESEYYKNKI |
| 179 | MPILTRTIELIPIGDKEERDRCYKWIRDFMEEQSKMMNQYMSALYIAAVEEVSKDDRKELNNLYN |
| | RIATSKKGSAFSKEECNLPKGLGANYGQRVRSDFDTACENGLLHGRVSLPTYKKNFPIILAPIYV |
| | NLQKNNIEEKGKSAGFYHNYASYNELYDALKEENKPEIIWNFVQKMQYQIKFGNPYKSAFLRDEI |
| | LHFLEGEYKAVGSQLSINSRGKIILNLSLDVPQKKVKLDENIVVGIDIGLAVPVMCAINNDYYKR |
| | LAVGDFEAFTRMREKLYSQKCKLQRQLKYTSGGHGRKKKLASLNAIRDREHRFVHTMNHKYSSEV |
| | INFALKNNAKYINMEDLTGFGKDNKGNAIDDYQFVLRNWSYFELQKMIQDKAQKYGIVVRKVESA |
| | YTSQLCSCCGEMGERVSQSVFRCLNPNCISHNKYEKQRKSGVGNYHFNADFNAARNISMSTNYTK |
| | KKRKTKAEKVEERKKNAIEKTAG |
| 180 | MVKMKKETKQKRNTLCKKITLYPLGSKEEVTRVYNFIRDGQYMQYKGLNLIMGQLASQFYKCNSE |
| | LDDPQYKEWVEQEIRNTNSLLQAMEFPKGIDSRSLIIQRVKQDFSSALKNGLARGERSITNYKRT |
| | LPLMTRGRDLKFSCNYSELEELEYKCFSKDFKVYVSWVNKIKFKVVLGSVKRSLALRKELCQILR |
| | SVYSIQGSSIEIKDKKIILNLSMGIPLMEKVLDENTVVGVDLGIAVPAVCGLNTNKNTRRFIGSK |
| | DDLLKIRTKVQSQRKSLNKKLRECTGGHGRSKKLQALDRLSESETNYVKTYLHMVSKEIIRFAVA |
| | HNAKYINIEKLEGYDASSFILRNWSYYQLQSFIEYKAKIKGIIVRKVNPYHTSQICSYCGHWEEG |
| | QRLSQDKFKCKCCGVELNADFNASRNIALSTEFVE |
| 181 | MAKDTFTITRKIQLVPVGDKTEVNRVYNYIREGMKAQNLAMNQYISALYLGMQNDVSKDDRKELN |
| | NLFSRISTSKKGSAYDESIQFAKGLPIGSMTRKVKSDFDTAMKKGLKYGKLSLPTYKDSNPLLVH |
| | VDYVRLRSTNPHQDSGLYHNYTNHTEFLEHLYKSDFELFIKFANYITFKIILGNPHKSAEIRDVF |
| | KNIFEECYAIQGSSIGIYNNKIICNLSISIPKKQLCLDENIVVGVDLGLAVPAVCALNTVPYIHK |
| | SLGNYDDFVRERTKMQSQRKRLQKSLNYANGGHGRKKKLQSLERLKKRERNWVQTYNHKISKQIV |
| | DFAIKNKAQYINIEDLSGFDSSQFVLRNWSYFELQQFIEYKANKYGIIVRKINPYHTSQTCSFCG |
| | HWEEGQRISQSEFICKNPECVNHNKSINADYNAARNIAMSTDFVKNN |
| 182 | MDNTITITRKYALIPEFSDRKEWKKRVYDFTINDLEQKIDYRNKKKQDASELESQLEYIKNGGDF |
| | TRNMVNNYTYSLVRTAMEEEARRKNYILSWIFSEMRANRVDQMESLKDKFKFVSDTINYAYRKAG |
| | SNKGSLFDETEIHCILKSYGIAFSQELTKEIKELVKNGVLEGKVVIPTYKLDSPFTIAKSHFSFE |
| | HDYDSFEELCEHISDSDCKMYMNYGGDNRKDGINPASIAKFKISIGHGENKDELKSTLLKVYSGE |
| | YQYCGSSIQITKNKIILNLTMKIPKIETKLDENTVVGVDLGIAIPAMCALNNNMYERLAIGSADD |
| | FLRTRTKLQSQRRRLQKSLKNSNGGHGRNKKLKVLERLGKSETHFVETYCHMVSKRVVEFAVKNR |
| | AKYINIENLNGYDTSQFILRNWSYYKLQQYITYKAERYGILVRKINPCYTSQVCSVCGNWEEGQR |
| | KTQSSFECANPECKSHEKYKYGFNADFNAARNIAMSTLFMETGNVTEKSKEEARKYYGIEKD |
| 183 | MDNTITITRKYALIPEFSDRKEWKKRVYDFTINDLEQKIDYRNKKKQDASELESQLEYIKNGGDF |
| | TRNMVNNYTYSLVRTAMEEEARRKNYILSWIFSEMRANRVDQMESLKDKFKFVSDTINYAYRKAG |
| | SNKGSLFDETEIHCILKSYGIAFSQELTKEIKELVKNGVLEGKVVIPTYKLDSPFTIAKSHFSFE |
| | HDYDSFEELCEHISDSDCKMYMNYGGDNRKDGINPASIAKFKISIGHGKNKDELKSTLLKVYSGE |
| | YQYCGSSIQIAKNKIILNLTMKIPKIETKLDENTVVGVDLGIAIPAMCALNNNMYERLAIGSADD |
| | FLRTRTKLQSQRRRLQKSLKNSNGGHGRNKKLKVLERLGKSETHFVETYCHMVSKRVVEFAVKNR |
| | AKYINIENLNGYDTSQFILRNWSYYKLQQYITYKAERYGILVRKINPCYTSQVCSVCGNWEEGQR |
| | KTQSSFECANPECKSHEKYKYGFNADFNAARNIAMSTLFMETGNVTEKSKEEARKYYGIEKD |
| 184 | MNLHGYKTDAKGFSSFLQSGNPVNLHGCKTRKEIKMESTTVITRKYTLIPEFSECKEWKKRVYDF |
| | TVKDIEQRIEYKKKKKQDISDLEIQLECIKNNSEFTRSMVNNYTYNLVRTAMEEEARRKNYILSW |
| | IFSEMTANRVDQMESLKDKFKFVSSTINYAYRKAGSNKGSLFDETEIHCMLKSYGIAFSQELTKK |
| | IKELVKNGVLEGKVVIPTYKLDSPFTIAKAHFSFDHDYDSFEELCEHINDSDCKMYMNYGGNNTK |
| | TGTNPASIARFRINLGHGKNRDELKATLLKVYSGEYRYCGSSIQISKNKITLNLSMKIPKVEMKL |
| | DENTVVGVDLGIAIPAMCALNNNLYERESIGKVNDFTRVRKKHQAQVKRLQKSLKSATGSHGRSK |
| | KMRALNRVKKSEAHFVESYCHYVSRKVVDFALKHNAKYINMENLKGYDTSQFILRNWSYYKLQQY |
| | ITYKADRYGIVVRKINPCYTSQVCSVCGHWEPDQRKTQASFECANSECESHKKYKYGFNADFNAA |
| | RNIAMSTLFMEDGEVTEKKKEEAREYYGIKK |
| 185 | MPTITRKIELTLCTEGLSDEQRKEQWKLLYHINDNLYKAANNISSKLYLDEHVSSMVRMKHAEYL |
| | SLLKELARAEKQQTPDEGLIAELSRKLSAAEKEMADQELAICKYATEMSTQTLSYNFAKEIETNI |
| | FGQILTCLRQGVYATENSDAKDVKRGERAIRNYKKGMPIPFPWNNSLKIEADSGEFYLRWYNGLR |
| | FLLTFGKDRSNNRMIVKRCMKMDEDFEGEYKLCNSSIQLAKRDGKPKLFLLLVVNIPQEHVELNK |
| | KIVVGVDLGVNVPAYVATNITEERKAIGDREHFLNTRMAFQRRYKSLQRLKGTAGGKGRTKKLEP |
| | LERLRDAERNWVHTQNHLFSREVVNFAVQARAATIHMEDLSGFGKDKDGNADEKKEFVLRNWSFY |
| | ELQNMIAYKAAKYGIKVVKIRPAYTSKTCSWCGQQGDRKSTTFICENPECKHYGESIHADYNAAR |
| | NIANSNDIVKENE |
| 186 | MDNTITITRKYALIPEFSDRKEWKKRVYDFTINDLEQKIDYRNKKKQDTSKLESQLEYIKNGGDF |
| | TRSMVNNYTYSLVSTAMEEEARRKNYILSWIFSEMRANRVDQMESLKDKFKFVSDTINYAYRKAG |
| | SNKGSLFDETEIRCILKSYGIAFSQELTKEIKELVKNGVLEGKVVIPTYKLDSPFTIAKSHFSFE |
| | HDYDSFEELCEHISDSDCKMYMNYGGDNKKDGINPASIARFRISLGHGKNKDELRATLLKVYSGE |
| | YQYCGSSIQITKNKIILNLTMKIPKIETKLDKNTVVGVDLGIAVPAMCALNNNMYERLAIGSADE |
| | FLRVRTKYQAQRRRLQKSLKNSSAGHGRKKKLKALDRMDKAESHFVETYCHIVSKRVVEFAVKNR |
| | AKYINIENLNGYDTSQFILRNWSYYKLQQYITYKAERYGIAVRKINPCYTSQVCSVCGNWEDGQR |
| | KTQASFKCANQKCESHKKYKYGFNADFNAARNIAMSTLFMEDGEVTEKKKEEAREYYGIKKENSK |
| | AV |
| 187 | MSYTEQFYIDKIAYYKNKLTKTKGKEEKQKIKDKLASLEEQENEFEESGILTQANVTDYTYDLVR |
| | NAMASEANRKNAIISYIILELLHNGGQTMDFNARNKLINDLVNYGLRVKGSSKGSLFDELDIENP |
| | LNAYGFAFKQDLKKKIRDMVNSKRVLDGKSSVITYKADSPFSINKENMSFTHDYSSFEELSDHIR |
| | DNDINLYFNFGSSGNPTIARFKINLGAGRHKKNKDELIATLLKLYSGEYQFCGSRIGIEKNKIIL |
| | NLVLSIPKKVRALDENTVVGVNLGVAVPAMCALNNNEYERLAIGSADEFLRVRTKLQAQRRRLQK |
| | SLKDASGGHGRTKKLKALERVAKAESHFANTYCHMISKRIVDFALKNNAKYINLENLTGYDTNDF |
| | ILRNWSYYKMQQYTTYKAEKYGIIVRKVNPCYNAQACSVCGNYAPGQRKSRAVFICANPACKSHK |
| | KNHGKLDAEFNNARNVAMSTLYMNDGQVTEKSFKEARDYFGIEEEIETI |
| 188 | MAKKQNKNTMQINRKITLIPVASDNDGWKKKMNTYLEKFYFDKKIKAKERQIKNTSKPERKEEYK |
| | QQLTEYKKQQEQFLNGELEDYTKQMVMSYTYDLVRDCMESEARQKNFIMSYMFSEMIREKVCYLK |
| | NKKEKEKWVNDNINVAFRVKGSPKGSIFDDVEIYSPLRALSIQGYTQELKGRVKKFATDGGLDGK |
| | LAVDNYKLDSPFHMSKQGFDIVHEYEDLTELKKNIGKSSCEIYVNLGNGGVPTFARFQLDFGHKG |
| | NREELISTITKVFTGEYKVCGSSIQINKKNKIILNLGLEIPKRLTELDENTVVGVDLGLAVPAVC |
| | SLNNNQYKKEYIGDGEAFVKQRGKIQKEKQRLQKALKLSKGGHGRKRKMLALERFKEREANFVNT |
| | YCHRISKKVVEYALKNNAKYINIENLKGYDSSKFILRNWSFYQLQQDITYKAERYGIEVRKINPA |
| | FTSQVCSFCGYWESGQRINQKTFKCGNPNCKSHNLKFFNADYNAARNISMSTLFTSDNYKFGKES |
| | LQKAADYYGIDLEIDSEDQEIA |
| 189 | MITVRKIKVRCEDKTFYDFMRKEQREQNKALNLSIGYIHTNSILKSVDSGAETLILNSIEKLNKK |
| | VDKLKKDLEKPKITDKKREQTEKAIQTNLKLIKDEQIKLEEGKQFRQGLDKQFSEIYINNNNLYH |
| | VLKSQTQVQYMRTLDLVTQKVKQDYSNNFVDIVTGKCSLMNYKSDFPLMIDKKCINIFKEEQNYK |
| | IRIMLGYELDIILGRRNNENVNELKSTLEKCISGEYKICQSSISINKNDVIFNLTLDIPNTTNYE |
| | PVHGRVLGVDVGVKYPVYMCVSDNTYKRKHIGSAADFLKVRQQFQERKRKLQASLDITKGGKGRK |
| | KKTQALERFKDKERNFAKTYNHQVSKKVVEFAKKNKCETIALEKITKEGIGDTILRNWSYYELQH |
| | MIEYKAKRECIKVEFVDPAYTSQTCSKCGHVSKDNRQTQEHFKCVNCGYELNADHNAAINIARRS |
| | IDYKPKEIIEKTIEKTIQPITTDLVEETGEFKQLTFI |
| 190 | MQRSITLKIIRASDDKITWEELGYLLRGLSLKICRMSNFCMTHHLLHALKLETEMLNPKGNLYCY |
| | PHLAEEYPEVPAGIVCAAESRARKLFRRCGAKVLRSEMSLPSFRKDNSIPIPVAGYRILQDGDTN |
| | YAEIQLLSRQGAKTQKLPGRIRLTLADNWRDKAARPALQKLAAGKIRRGVASLYRAKNDWYLCIP |
| | YEVEAANTGGDFEPGLVMGVAFGVYNALVYGENTLLKRGAISGEEILSHQAKVKARRKKIQEQYP |
| | WSGRKGQGREDTLKPLRHLHEAEKNYRELVNHRYAKWVVDIAVKNRCGEIHLDDGHAPPLGKNKI |
| | LLSRWSLYDLKNKICRKAEEKGIRVTECSVPDLRTRCSHCGTEQVAGNHKRMFLCADCGYGSTEK |
| | NRGTGYISVDYNAARNLAMYDTGKNEPNMERDLIPKDDPAYDRNEGFREELSGSGQ |
| 191 | MATEYTCNTRKIEVHLHRHGEDEEAKQRLIDDYRVWDTINDNLYKAANRIVSHCFFNDAFEYRLK |
| | IHSPRFQEIEKLLKYPKRNKLTDEDIKQLKAERKQLFADFKKQRHTFLRGGVAEGANPEQNSTYK |
| | VISNEFLEVIPSEILTNLNQNISSTYKNYSLDVERGIRTIPNYKRGIPVPFSIKQRGELMLKRRD |
| | DGSIYVRFPLGLEWDLSFGRDRSNNREIVERVLSGQYDVGNSSIQESKNRKRFLLLVVKIPKENH |
| | NLNPDRIVGVDLGINIPLYAALNDNEYGGMGIGSREQFLNMRMRMVAKKRELQRNLRQSTNGGHG |
| | RAQKLQALERFEGKERNWVHLQNHIFSKSIIEYAVKNNAGTIQMERLTGFGRDKNDEVDSDFKFI |
| | LRYWSFFELQTMIEYKANAAGIEVRYVDPYHTSQTCSFCGHYEKGQRLNQSTFVCKNPDCEKGKG |
| | KKLSNGTYQGINADWNAARNIALSDKIVDRKKK |
| 192 | MLITRKIELHISEADPELRKEHWKYLSFLNSEIYKAANLIVTNQLENNFLENRVVDADGNVMDIG |
| | KRIRSLYRNKDKNADEIEKLKAAKLELRTEVKKFYLKSKQNTTYSITSHNFPEIDASIITTLNAQ |
| | ITGVLKKEWNEVERGSRSLRTYKKGMPIPFNLTSKSKKWFEKVDDEIFLTWFKGIKFKLFFGRDR |
| | SNNRAIVDRCLSGEYKYSDSSIQYKDRKIFLLLVVDIPESTKVLDENVSVGVDLGITVPAYCALS |
| | DGLKRLAIGSSEDLLRVRLQFQSRKRRLQRALKSSKGGQGRDKKLKALDNVADKEKRYVSTYNHM |
| | ISSNIVKFAKDNNAGVIKLEMLEGFGEDEKNKFILRNWSYYQLQTMIQYKAKRENIKLVFIDPYH |
| | TSQTCSICNHYEAGQREKQSEFICKNPDCKNFDTPINADFNAALNIAVSKKVVTSKEDCEYFKKE |
| | N |
| 193 | MILTRKIKLVIVSENQKEGYSLIRNEIREQYKALNLAYNHLYFEHNAIQKLKQNDEDYKQKRSKL |
| | QELINKKYEEHQKVKNLEKKEALREAYNKKKQELYNFEKEYNEKARQAYQQVVRFTQQTRVRNLI |
| | NRECNLMSDTKDGITSKVTQDYKNDCKAGLLIGKRSLRNYKKDNPLLVRGRSLKFYKEDGDYFIK |
| | WNKGTIFKCILHIRKKNVAELQSVLENVLLGAYKICDSSIGFNNKDMILNLSLNIPDKETHDYIP |
| | GRVVGVDLGLKIPAYVSLSDKVYVRKGIGSIDDFLRVRTQMQKRRRRLQESLAAVKGGKGREKKL |
| | KALDHLKGKEANFAKTYNHELSTQIVTFAVKNQAGQINMEFLEFDKMKNKSLLRNWSYYQLQMMV |
| | EYKAKREGIIIKYVDAYLTSQTCSKCDHYEEGQRETQERFKCKSCGYEVNADYNASRNIAKSTRY |
| | ISDSTESEYHRKKQEALKEILGENDTINEQLSLFDKRDDIA |
| 194 | MQRSLKLKIVRPYDESITWEEIGYLLRGISYQICKMSNYCMTHHLLRALGMETENLNPQGNLYCY |
| | PRLAKEYPDVPTGIICAAEGRARKLFKRNAPGILRSETALPSFRKESSIPIPVAGYSLAKIGPDI |
| | YVADVQLLSRKAAKTGKLPGRIQFVLANNWRDKKAGSVLHQLAEGTIKRGIASIFRNNRDWYIGI |
| | PYSVDPAPSDGELDPDLVMGVAFGTHSALAYAFNNLLKRGELGGEEVLSHREKFKARRQHIREQY |
| | NWSGRKGHGRENALKPIRELYEKERNYRNLTNERYAKWVVEIAKKNHCGKINLDAGGDRRNGKQN |
| | ILLAYWPQSELHMKIKNKAEAYGIQVQECTATDIRRRCSRCGAVQETSGDKRWFICSHQGYGKEE |
| | KKTSSGFVSVDYNAARNVSMWEGTT |
| 195 | MIAVRKLKVICKDKEFYDFFNMEQREQNKALNMAISLRHVNNTLKRIDSGAEVIIIKSIDKLNRK |
| | IETLEKELKNENITEAKKEKTLKDIETHKKIIKGEKKKLEEGRIYRKGLDKEFSQNYFDKTQLYH |
| | VLDGMTSIQHKRTIELVREKVKKDYENNFIDIVTGKQSLPNYKSDFPLMIDGSSIHIFEEDGIYK |
| | IKIMRGYELEVILGRRENENILELRKTLDRCSNGQYKVCQSEIQRDKNNNIIFNLTIDIPIENKK |
| | YTPVEGRVLGVDLGIKYPVYMCLNDDTYKRTSLGNINNFLRVREQMQERRRAFQKDLTLTKGGKG |
| | KSKKTQLLNKLRENEKNFCKTYNHTMSKRVVEFAKKHNCEYIHIENLTKDGFSNSILRNWSYYEL |
| | QKFIKYKADREGIVVKYIDPAYTSQMCSKCGYTDKENRQTQAEFKCISCGFKLNADHNASINIAR |
| | SNKFIK |
| 196 | MPTITRKIELNLCTEGLADEEQKAQWNLLYHINDNLYKAANNISSKLYLDDHVSSMVMLKHAEYL |
| | TLVRALEKAQKQKTPDETVIEDLRRQVAAAEKDMTDQELAICNYATEMSTQSLSYRFATEIETNI |
| | FAQILDCLKQGVYATFNSDAKDVKRGERAIRNYKKGMPIPFPWNKSLKIEHKDGEFYLLWYNGLR |
| | FHLNFGKDRSNNRLIVQRCMKMDKDYEGDYKMCNSSIQFVKREGKPKFFLLLVVNIPQEHVELDK |
| | KIVVGVDLGINSPAYVATNVTMERQQIGRRETFLNGRMSFQRRYKSLQRLQTTAGGRGRKKKLEP |
| | LERLRNAEHNWVHTQNHLFSREVVQFAVKARAATIHMEDLSGFGKDNDGNADERKEFVLRNWSYY |
| | ELQTMISYKAQKYGIKVEKIRPAYTSRTCSWCGHEGFRKGEIFICENPACEKCGEKENADYNAAR |
| | NIANSKDIIKNHG |
| 197 | MGNNRMTICRKIKLFPVGDKDEINRVYDFIRNGQYAQYQACNLLMGQLMSEYYKYNRDIKNEEFK |
| | ARQKEIMTNSNIILKDIDFATGVDTPSAVTQKVKQDFSTALKNGLAKGERTVTNYKRTNPLITRG |
| | RNLTFYHEYETYHDFLDKINDSDLAVYVKWVNKIVFKVVFGNPHRSLELRSVIQNILEENYKVQG |
| | SSIEIDGKSIILNLSISIPKQLRELDENIVVGVDLGIAVPAMCALNNNLYERLAIGNADDFLRIR |
| | TKMQAQRKRLQKSLRNTSGGHGRAKKLKALERLQKAEVHFVETYCHMISKRVVDFALKHNAKYIN |
| | IENLTGYDTSDFILRNWSYYKLQDYITYKAAKYGIEVRKINPCYTSQICSVCGNWEFGQRKSQSV |
| | FECANENCDSHKKYEKTGFNADFNAARNIAMSTLWMGSGQVTEKSKQEAREYYDISEKYEQSKNG |
| | SENNKVA |
| 198 | MNIVKKIKLRIIDNDKELCKKQYLGFTEEQKKELIDKQYKFIRDSQYQQYLGFNRAMGFLMSGYY |
| | ANNMDIKSDNFKEHQKKLTNSLYIFDDIKFGVGIDSKSLIVQRVKKDFSTALKNGLAKGERSVTN |
| | YKRTYPLLTRHRSIKFLYAENELDIYLDWVNKIRFRCELGNHKNSLELQHTLRKVITGEYKISDS |
| | SLEFNKKNELILNLNLNIPETKATFIKDRTLGVDLGMAIPAYVSLSDTPYIRKGFGSYEEFAKVR |
| | NQFKDRRKRLLKQLSLVAGGKGRAKKLHSMEFLKNKEKQFAKTYNHSLSKKIIDFALKNNCEYIN |
| | LEDIKSTSLEDRVLGQWGYYQLQEQIEYKAKLVGIKVRKVKAAYTSQTCSECGNIDKENRKNQST |
| | FKCTNEDCKLNKKGINADWNASINIARSKEFIK |
| 199 | MATDYTVITRKIEVHLHRHGDSEEAIQRYKEEFRIWDEINDNLYKAANRIVSHCFENDAYEYRLK |
| | IHSPRFQEIEKLLRYSKRNKLTDEDIKQLKNERKQLFTDFKKQRLAFLRGGATEGTNPEQNSTYK |
| | VVSNEFGDIPSDILTCLNQNIASVYKAYSKEVEFGMRTIPNYKKGIPIPFTIRPNGVLNLQKRED |
| | GSIYIRMPKGLEWDLSFGRDRSNNCEIVERVLSGQYDVGNSSLQESKNKKRFLLLVVKIPKVNKA |
| | LNQDRVVGVDLGINTPLYAALNDNEYGGMSIGSREQFLKMRMRMNAQKRELQHNLRHSTNGGHGR |
| | KQKLQALERLEGKERNWVHLQNHIFSKSIIEYALKNDAGVIQMERLTGFGRNKNDEVDEGYKFIL |
| | RYWSFSELQNMIEYKANAAGIEVRYIDPYHTSQTCSFCGHYEKGQRISQSTFVCKNPECTKGKGK |
| | HKSDGSYEGINADWNAARNIALSNNIVGRKKK |
| 200 | MATEYTCITRKIEVHLHRHGDSEEALQRYKEEFRIWDEINDNLYKAANRIISHCFENDAYEYRLK |
| | LHSPRFQEIEKLLKYAKRNKLTDDDIKALKAERKELFAEFKRQRQSFLGGSEQNSTYKVVTDEFL |
| | EVIPSHVLTCLNQNISSTYREYALDVEHGRRTIPNFKKGIPVPFPIKATGELLLRKREDGSIYIR |
| | FPKGLEWDLNFGRDRSNNREIVERVLSGQYDVGNSSIQETKNRKRFLLLVVKIPKESRALNPDRV |
| | VGVDLGVAVPLYAALNDNPYGGMSIGSADQFLKVRMRMAAQKRELQRTLRNSTNGGHGRKHKLQA |
| | LDRLEGKERNWVHLQNHIFSKELVEFALRNEAGAIQMERLTGFGHDRNDEVDEGFKFILRYWSFF |
| | ELQTMIEYKAKAAGIEVRYVDPYHTSQTCSFCGHYEKGQRVNQATFICKNPDCTKGKGKERSDGT |
| | FEGINADWNAARNIALSDKIVERKKK |
| 201 | MVEQGVTITLNKSDKNTYTIARKVRLAVDGDKEEVNRVYQFLRDGIYNQNKAYNIFISSVYSAII |
| | NGASTEELNEIYKRGSRKPKEDDETYSLYKFGEIEFPVGVGTTASLKQMVKNDLKKAKNDGLFKG |
| | KISLPNRKLNAPLRIESACFSFIHNYNSYQEFLDHLYTDDCEIIMKFVNNIRFKVNLGQACKTHE |
| | LRSVFQNIFEENYKVCGSSIEIDGTKIILNLSLTIPKKKHELDESIVVGVDLGIAVPAVCSLNNN |
| | TYIKKFIGSRNDFLRERTKLKAQRRNVQKSLKFTSGGHGRKKKLRHLEVFTEHEKNWVKNYNHRV |
| | SKEIVDFALSNDAKYINIENLQGEDSSNELLANWSYYQLQQQIEYKADMHGIIVRKVNPYHTSQR |
| | CSCCGFESPDNRPKDKKGQAYFKCLNCGTEMNADFNASQNIARSTDFSIGEVTLEEDKKKHNKSK |
| | KIPKEKQAA |
| 202 | YISSHMFFNEAYIERLKAHSHRYREIKKTLKKIDEFTPEEAKALNDEKRVLEKQFVEERLRFLKG |
| | GDANGSGSVMSSTRQAVVEAFPEIPFKVLDCLNREMSKTFSQYKRDIENGKRTLSNYKKYYPVPF |
| | SMEQGKQLRKREDGSTYVMFPGKLEWDLYFGKDPSNNREIVERIFNGEYQACDSSIKEIKGRKKQ |
| | ILHLVVKIPKKTIKRDSNRVVGIDLGVNTPLYAALNDSERERFSIGSREAFLNVRMRFNAQRREL |
| | QRNLRHTTNGGHGRKQKLQALERLEGKERNWVHLQNHIFSKSLIEFAQKCEAGVIQMEELSGFGR |
| | DKFDSVDDGYKFILRYWSFFELQNMIEYKANAAGIEVRYVNPAYTSQTCSYCKHYEKGQRISQST |
| | FVCKKCTEGKGKQRKDGSYEGINADWNAARNIAMSDKIVDRKKK |
| 203 | MITTRKLKLTILGDEETRKLQYKLIRDEQYEQYKALNLCMSLLNTHNILSGYNTGAENKLNSQID |
| | KLNKKLEKAQFEIKKNDIKQSKLDKLNSDIELYQNELNKLKEQFTQSSKYRSDIDLKFKEMYIDD |
| | LYTIVQNQVTFKNKDLMSLVTQRAKKDYSIALKNGMARGERSLTNYKRDFPLMTRGERWLKFKYD |
| | ENSDDILIDWISGIKFKVILGYRKNENSIELRHTLHKVINKEYKICDSSIQFDRNNNLILNLTLD |
| | IPNNSKSEFIENRTLGVDLGIKYPAYICLSDDTFKRESIGCAEDFIRVREQIRNRRKRLQQQLKM |
| | VQGGKGREKKLKALDRISDKERNFVKTYNHMISKNIVNFAKKHKCQYINLEKLTKDGFPNMILSK |
| | WSYYELQQMIEYKAERENIKVRYIDPAYTSQTCSRCSHIEKDNRETQEKFKCLKCEFELNADHNA |
| | AINISRSVNFK |
| 204 | MISTRKIKVRCDDNTFYTFFRQEQREQNKALNIGMGIIHADAILHNIDSGAEKKLKKSIEGLQGK |
| | IDKLNKYLEKENITDKKKKEVLKAIETNKKILDGEKKAFKESEEYRKGIDELFKSTYLKSNTLYH |
| | VLDSMVNIQYKRTLSLVTQRIKKDYSNDFVGVVTGQQSLRNYRNDNPLMISNQQLDLKYIEDTFY |
| | LDVMCGYRLEIILGRRDNQNVNELKSTLHRILSKEYKVCDSSMQFDKNNKDVILNLIIDIPNKSN |
| | MYEAIKERTLGIDLGVEVPIFMCLNDNTYIKKGIGDINNFLRVRQQIQIRRRKLQKDLTLTNGGK |
| | GRKKKLQLLEKLQENEKNFVKTYSHVLSKRIIEFAKKNKCEYINLEKLTKDGFDNIILRNWSYFD |
| | LQRMIEYKAKREGITVRYVNPSFTSQKCSKCGEIDKENRQTQADFKCTNCGFELNADHNAAINIA |
| | RSIDFV |
| 205 | MQRSIKLKILRPFDGNITWEALGYLLQGLSFKVCRISNFCLTHHLLRALKLETENLNPKGHLYCY |
| | PRLAEEYPEVPAGIICAAEGRARKVFNQKGKSIMRSEMALPTFRKNCSIPIPTAGYNLRWEGKDT |
| | CIAEVQLLSRQGAKTGKLPGRISLVLADNRRDKTAGAVMRKLAEGTLRRGAATLFREKKDWYISI |
| | PYETEAIHTEKDFVPGLVMGVAFGIRCTLAYGFNRLLKRGEIKGDEVLAHQEKYLARRKKIQEQY |
| | NWSGRRGHGREHALKPLRHLYEKERNYRSLVNARYAKWIVEIAEKNRCGAIHLDGENRLQRGKYP |
| | ALLARWPLEELRQKIREKAELYGIKVSECTEAGIRERCSRQGAVQEDAADGYKFTCTACGYGAKG |
| | NNSSTGYISVDYNAARNLAVWEPETDS |
| 206 | MATEYTCITRKIEVHLHKHGDSEEALQRLNEECRIWDEINNNLYKAANRIISHCFENDTYEYRLK |
| | LQSPRLQEIEKLLSNPKRNKLSDEDIKQLKAERKQLFANFKKQRQVFLRGGVEEGANPEQNSTYR |
| | VVSNEFIDVIPSEVLTNLNQNISSTYREYSLDVERGSRTIPNYKKGIPVPFSIKRSGELMLKKRE |
| | DGSIYVRFPKCLEWDLFFGRDRSNNREIVERVLNGQYDVGISTIQETKNKKRFLLLVVKIPKESK |
| | KLNPNRVVGVDLGINIPLYAALNDNEYGGLGIGSREQFLKVRMRMVAQKRALQRNLRHTTNGGHG |
| | RAQKLQALDQLEGKERNWVHLQNHIFSKSIIEYALKNGAGVIQMERLAGFGRDKNEEVENEFKFI |
| | LRYWSFFELQTMIEYKANVAGIEVRYIDPYHTSQTCSFCGHYEKGQRINQSTFVCKNPDCVKGKG |
| | KQHADGSYDGINADWNAARNIALSTTVVD |
| 207 | MQRSIKLKILRPFDGNITWEALGYLLQGLSFKVCRISNFCLTHHLLRALKLETENLNPKGHLYCY |
| | PRLAEEYPEVPAGIICAAEGRARKVFNQKGKGILRSEMALPTFRKNCSIPIPTAGYNLRWEGKDT |
| | CIAEVQLLSRQGAKTGKLPGRISLVLADNRRDKTAGAVMRKLAEGTLRRGAATLFREKKDWYISI |
| | PYETEAIHTEKDFVPGLVMGVAFGIRCTLAYGFNRLLKRGEIKGDEVLAHQEKYLARRKKIQEQY |
| | NWSGRRGHGREHALKPLRHLYEKERNYRSLVNARYAKWIVEIAEKNRCGAIHLDGENRLQRGKYP |
| | ALLARWPLEELRQKIREKAELYGIKVSECTEAGIRERCSRCGAVQEDAADGYKFTCTACGYGAKG |
| | NNSSTGYISVDYNAARNLAVWRQETNS |
| 208 | MGNDRMTICRKIKLFPVGDKEEINRVYDFIRNGQYAQYQACNLLMGQLMSEYYKYNRDIKNKEFK |
| | ARQKEIMTNSNILKDIDFVTGVDTPSAVTQKVKQDFSTALKNGLAKGERTVTNYKRTNPLITRGR |
| | NLTFYHEYETYQNFLDKINDSDLAVYIKWVNKILFKVVFGNPHRSLELRSVVQNILEENYKVQGS |
| | SIEIDGKSIILNLSISIPKQLRELDENIVVGVDLGIAVPAMCALNNNIYERLAIGNADDFLRIRT |
| | KMQAQRKRLQKSLRNTSGGHGRAKKLKALERLQKAEVHFVETYCHMISKRVVDFALKHNAKYINI |
| | ENLTGYDTSDFILRNWSYYKLQDYITYKAAKYGIEVRKINPCYTSQICSVCGSWEFGQRKSQSVF |
| | ECANENCDSHTKYERGFNADFNAARNIAKSTLWMESGQVTEKSKQEAREYYGISEKYEQSKNEVE |
| | NNKVA |
| 209 | MQRSIILKIIRPYDESIKWDDVGYLWRGLSFKVCKISNYCMTHHLLRAMNLETENLNPQGRLYCY |
| | PHLAKEYPEIPAGICAAEGRARKVFKQNAKGILYSETSLPSFRKDCSIPIPVSGYSLLKAGADTY |
| | VASIQFLSRQAAKTQKLPGRIQLVLASNWRDKSAGRILQQLAEGTLKRGIASLFRKKRDWYFSIP |
| | YEVEPSGADDSFEPELAMGVVFGFQCALAYGFNRLLKRGMLGGDELLAHREKMLARKKQILTQYR |
| | WSGRKGHGRESALKPLQALYEKERNYRNLTNERYAKWIAEIAKKNRCGKIYLDTGGSSGHGTQNI |
| | LLAYWPKEALRKKIMNKAEAYGIEVVECTDAEIRNRCSRCGTLQEPLENKKWFVCNHCGYGKDDK |
| | KTGDGFISVDYNAARNLAVYDTGSKESV |
| 210 | MISKRKIKVRCDDNTFYTFFRQEQREQNKALNIGIGIIHSNAILHNIDSGAEKKLKKSIESLQGK |
| | IDKENKHLEKEKLTDKKKEEVLKAIETTKKILDGEKKAFKKSEEYRKGIDELFKSTYLKSNTLDH |
| | VLDSMVNIQYKRTLSLVTQRIKKDYSNDFVGIITGQKSLRNYRNDNPLMISNQQLDFKYIEDTFY |
| | LDVMCGYRLEIILGRRDNQNVNELKSTLHRILSKEYKVCDSSMQFDKNNRDVILNLGIDIPNKSN |
| | MYEAIKGRTLGIDLGVEVPIFMCLNDNTYIKKGIGDINNFLRVRQQIQVRRRKLQKDLTLTNGGK |
| | GRKKKLQLLDKLQENEKNFVKTYSHALSKRIIEFAKKNKCEYINLEKLTKDGFDNIILRNWSYFD |
| | LQRMIENKAKREGITVRYVNPSFTSQKCSKCGEIDKKNRQTQADFKCINCGFELNADHNAAINIA |
| | RSLDFV |
| 211 | ITWEALGYLLQGLSFKVCRISNFCLTHHLLRALKLETENLNPKGHLYCYPRLAEEYPEVPAGIIC |
| | AAEGRARKVFNQKGKGILRSEMALPTFRKNCSIPIPTAGYNLRWEGKDTCIAEVQLLSRQGAKTG |
| | KLPGRISLVLADNRRDKTAGAVMRKLAEGTLRRGAATLFREKKDWYISIPYETEAIHTEKDFVPG |
| | LVMGVAFGIRCTLAYGFNRLLKRGEIKGDEVLAHQEKYLARRKKIQEQYNWSGRRGHGREHALKP |
| | LRHLYEKERNYRSLVNARYAKWIVEIAEKNRCGAIHLDGENRLQRGKYPALLARWPLEELRQKIR |
| | EKAELYGIKVSECTEAGIRERCSRCGAVQEDAADGYKFTCTACGYGAKGNNSSTGYISVDYNAAR |
| | NLAVWRQETNS |
| 212 | MQRSIILKIRPYDESIKWDDVGYLWRGLSFKVCKISNYCMTHHLLRAMNLETENLNPQGRLYCYP |
| | HLAKEYPEIPAGIICAAEGRARKVFQQNAKGILYSETSLPSFRKDCSIPIPVSGYSLLKAGADTY |
| | VASIQFLSRQAAKTQKLPGRIQLVLASNWRDKSAGRILQQLAEGTLKRGIASLFRKKRDWYFSIP |
| | YEVEPSGADDSFEPELAMGVVFGFQCALAYGENRLLKRGMLGGDELLAHREKMLARKKQILTQYR |
| | WSGRKGHGRESALKPLQALYEKERNYRNLTNERYAKWIAEIAKKNRCGKIYLDTGGSSGHGTQNI |
| | LLAYWPKEALRKKIMNKAEAYGIEVVECTDAEIRNRCSRCGTLQEPLENKKWFVCNHCGYGKDDR |
| | KTGNGFISVDYNAARNLAVYDTGSKESV |
| 213 | MATEYTCITRKIEVHLHRHGDSEEAAQRLKEEYRIWDEINDNLYKAANRIISHCFFNDAYEYRLK |
| | LHSPRFQEIEKLLKYAKRNKLTDDDIKALKAERKELFAEFKRQRQSFLGGSEQNSTYKVVTDEFL |
| | EVIPSDVLSCLNQNISSTYREYALDVEHGRRTIPNFKKGIPVPFAIKVHRELALRKREDGSIYIR |
| | FPKGLEWDLNFGRDRSNNREIVERVLSGQYDVGVSSIQEAKNGKRFLLLVVKIPKESRALNPDRV |
| | VGVDLGVNIPLYAALNDNTYGGLSIGSRDQFLNVRMRMAAQKRELQRNLRVATINGGHGRKQKLQ |
| | ALDRLEGKERRWVHLQNHIFSKSIIEYALRNEAGAIQMERLTGFGHDRNDEVDEGFKFILRYWSF |
| | FELQTMIEYKAKAAGIEVRYVDPYHTSQTCSFCGHYEKGQRVNQATFICKNPDCTKGKGKERSDG |
| | TFEGINADWNAARNIALSDKIVERKKK |
| 214 | MPIITRKIELKLCTEGLSEETIKSQRMLLYHINDNLYRVANNISSKLYLDEHVSSLVRMKNGDYL |
| | SLEKQLVKAKKQKNPDTKTITELEERILTVKREMNSQEIAISKYATEMSTKTLAYNFAKELGSDI |
| | FGQILACLQQNVHSTFSEDAQEVRRGERAIRNYKKGMPIPFPWNRSIRIEALDGEFYLRWYNGIR |
| | FHLFFGKDRSNNRQIVKRALCLDPDYSGENYKLCNSSIQLVKREHERKTFLLLVVDIPKEVRKLN |
| | KDIVVGVDLGINVPAYVATNSTEERKEIGDREHFLNERMSFQRRYKSLQRLKCTAGGKGRKKKLE |
| | PLERLREAEHKWVHTQNHRFSREVVDFALHAEAATIHMENLSGFGKDQEGNADEKKEFVLRNWSY |
| | YELQNMIAYKAAKYGIKVEYVKPAYTSKTCSWCGKEGFRQSTIFICENHECKMCGIKVHADYNAA |
| | RNIANSNNIIKNE |
| 215 | MATEYTCITRKIEVHLHRHGDSEEAAQRLKEEYRIWDEINDNLYKAANRIISHCFFNDAYEYRLK |
| | LHSPRFQEIEKLLKYAKRNKLTDDDIKALKAERKELFAEFKRQRQSFLGGSEQNSTYKVVTDEFL |
| | EVIPSDVLTCLNQNISSTYREYALDVESGRRTIPNFKKGIPVPFAIKVHRELALRKREDGSIYIR |
| | FPKGLEWDLNFGRDRSNNREIVERVLSGQYDVGVSSIQEAKNGKRFLLLVVKIPKESRALNPDRV |
| | VGVDLGVNIPLYAALNDNTYGGLSIGSRDQFLKVRMRMAAQKRELQRNLRVATNGGHGRKQKLQA |
| | LDRLEGKERRWVHLQNHIFSKSIIEYALRNEAGAIQMERLTGFGHDRNDEVDEGFKFILRYWSFF |
| | ELQTMIEYKAKAAGIEVRYVDPYHTSQTCSFCGHYEKGQRVNQATFICKNPDCTKGKGKERSDGT |
| | FEGINADWNAARNIALSDKIVERKKK |
| 216 | MQRSITLKIIRPEDETISWEELGYLLRGLSFKVCRMCNFCMTHQLLHALKLETELLNPQGNLYCY |
| | PRLAEEYPDVPTGICAAETRARKLFRRSAEAVLHSETSLPRFRKDSSIPVPVAGYKILQDADHNV |
| | YADVQLLSRQGAKTQKLPGRIRLVLADNWRDQSAKAALQQIVAGKVKRGVASLFRVKNDWYFQIP |
| | YVTEAVNTEEIFQPDLVMGVAFGLQDALVYAFSTSMKRGAVSGEEVLAHQEKYVARRKKIQEQYN |
| | WSGRKGHGREDALKPLRHLYETERNYRNLVNSRYAKWVVDIAAKNRCGVIHLDSANYVSSGKNIL |
| | LSRWPLYDLKEKIRRKAEEKGIQVTECSIPNLRTRCSRCGKEQEPEGEKRTFVCKDCGYGKADKN |
| | RRGGFITVDYNAARNLAVYESEDAKL |
| 217 | MATEYITITRKIEVYLHRHGDSDEAKQRYQQEWQIWHDINDNLYKAANRIVSHCFFNDAYEFRLR |
| | LHSPRFAEIEKALKHSKKNKLSDDEVKALKAERQELYKSFKQQRLAFLRGGATEGANPEQNSTYK |
| | VISDEFGDIIPSDILTCLNQNIASVYKAYSKEVQFGDRTIPNYKKGIPAPFSIKAGGALLLKKRE |
| | DGSIYVRMPKGLEWDLVFGRDRSNNREIVERILSGQYDAGNSSIQQAKNGKCFLLLIVKIPKSNI |
| | ALNKDRVVGVDLGINIPLYVALNDNEYGGMGIGSREQFLKMRMRMSAQKRELQRNLRHSTNGGHG |
| | RKQKLQALERLEGKERNWVHLQNHIFSKSVIEFAQKHNAGVIQMERLKGYGKDANGEVREEAKFL |
| | TRYWSYFKLQTMIEYKANAAGIEIRYIDPYHTSQTCSFCGHYEKGQRISQSTFICQNPECKQGKG |
| | KQKSDGTFEGINADWNAARNIALSEQYVDKKKK |
| 218 | MATEYTCITRKIEVHLHRHGDSDEALQRYKEEYRIWDEINDNLYKAANRIISHCFFNDAYEYRLK |
| | LHSPRFQEIEKLLKYAKRNKLTDDDIKALKAERKELFAEFKRQRQSFLGGSEQNSTDRVVSHEFL |
| | DVIPSDILTCLNQNISSTYREYALDVEHGRRTIPNFKKGIPVPFPIKATGELLLRKREDGSIYIR |
| | FPKGLEWDLNFGRDRSNNREIVERVLSGQYDVGNSSIQETKNRKRFLLLVVKIPKESRALNPDRV |
| | VGVDLGVNIPLYAALNDNTYGGLSIGSRDQFLKVRMRMAAQKRELQRNLRVATNGGHGRKQKLQA |
| | LDRLEGKERNWVHLQNHIFSKSIIEYALRNEAGAIQMERLTGFGHDRNDEVDEGFKFILRYWSFF |
| | ELQTMIEYKAKAAGIEVRYVNPYHTSQTCSFCGHYEKGQRVNQATFICKNPDCTKGKGKERSDGT |
| | FEGINADWNAARNIALSDKIVERKKK |
| 219 | MATEYTCITRKIEVHLHRHGDSEEAAQRLKEEYRIWDEINDNLYKAANRISHCFFNDAYEYRLKL |
| | HSPRFQEIEKLLKYAKRNKLTDDDIKALKAERKELFAEFKRQRQSFLGGSEQNSTDRVVSHEFLD |
| | VIPSEVLTCLNQNISSTYREYALDVEHGRRTIPNFKKGIPAPFSIKANRELALRKREDGSIYIRF |
| | PKGLEWDLNFGRDRSNNREIVERVLSGQYDVGVSSIQEAKNGKRFLLLVVKIPKESRELNPDRVV |
| | GVDLGVAVPLYAALNDNQYGGMSIGSADQFIKVRMRMAAQKREMQRTLRNSTNGGHGRKQKLQAL |
| | DRLEGKERRWVHLQNHIFSKELVEYALRNEAGAIQMERLTGFGHDRNDEVDEGFKFILRYWSFFE |
| | LQTMIEYKAKAAGIEVRYVDPYHTSQTCSFCGHYEKGQRVTQSKFVCKNPDCTKGKGKERSDGTF |
| | EGINADWNAARNIALSDKIVERKKK |
| 220 | MPTITRKIELTLCTDGLSDEERKAQWGLLYHINDNLYKAANNISSKLYLDEHVSSMVRLKHAEYL |
| | SLQKELAKAERQKMPDVDVIEELRERLSAAEQEMSDQELAICKYATEMSTNTLAYRFATEIETNI |
| | FGQILARLENNAQAVFLTDAPDVKRGERAIRNYKKGMPIPFPWNNSIKIECEGGEFYLRWYSGLR |
| | FHFNFGKDRSGNRLIVQRCLKLDKEYDGEYKLCNSSIQMVKRDGSTKFFLLMVVNIPQEYVELNK |
| | HIVVGVDLGINVPAYVATNITPERKAIGDREHFLNTRMAFQRRYKSLQRLKTTAGGKGRTKKLEP |
| | LERLRQAEHNWVHTQNHLFSREVVNFALQTHAATIHLEDLSGFGKDSDGNADERKEFVLRNWSYY |
| | ELQNMITYKAAKYGIRVEKIRPAFTSRTCSCCGHEGFREGVTFICENPECQQFGEKVHADYNAAR |
| | NIANSKDIIKKNE |
| 221 | MATEFTCITRKIEVHLHRHGDSEEAIARYKNEWDMWDEINNNLYKAANRIVSHCFFNDAYEYRLK |
| | MHSPRFQEIEKLLRYTKRNNLSDNDIKQLKEERKNLFADFKKQRLAFLQGNSGIGSEQNSTYKVI |
| | SNEFLDTIPSQILTNLNQNISSTYREYTQDIERGIRTIPNFKKGIPVPFSIKKGGDLMLKKRNDG |
| | SIYIRFPKGLEWDLNFGRDRSNNREIVERILNGQYDVGNSSIQETKNKKRFVLLVVKIPKENQKL |
| | DTNRIVGIDLGINTPLYAALNDNEYGGFSIGSRDQFLKMRMRMAAQKRELQRNLRHTTNGGHGRN |
| | QKLQAFKRLQDKEKNWVRLQNHIFSKSIIEYALKNKAGVIQMERLTGFGRDKNDEVNEDYKFILR |
| | YWSFFELQTMIEYKANAAGIEVRYIDPYHTSQTCSFCGHYEKGQRINQQTFICKNPDCTKGKGKQ |
| | EKDGTYKGI |
| 222 | MPTITRKIELTLCTEGLSDEQRKAQWGLLFHINDNLYKAANNVSSKLYLDEHVQSMVRMKHDEYL |
| | GLLKELARAEKQKMPDAAVIAELREKIAAAEKEMTDQELAICKYATEMPTQSLSYRLVTEIETNI |
| | FAQILDCLKQNVFATFNSDARDVKRGERAIRNYKKGMPIPFPWNKAIKIESQGDDFFLRWYSGIR |
| | FKFVFGKDRSNNRAIVMRCLKLDEDYDGEYKLCNSSIQMVKRDGGMKLFLLLVVSIPQEHVELNK |
| | KIAVGVDLGVNVPAYVATNITEERKAIGDREHFLNTRMQFQRRYKSLQRLKTTAGGKGRTKKLEP |
| | LERLRDAERNWVHTQNHLFSREVVNFAVQTHAATIHLEDLSGFGKDYDGNADERKEFVLRNWSYY |
| | ELQNMITYKAAKYGIHVVKVRPAFTSRTCSCCGQQGFREGVTFICENPECKQYGEKVHADYNAAR |
| | NIANSNDIIQDNHE |
| 223 | MITSRKIKLSIVSNNSTEAYNFIRKEMKVQNKALNVAMNHLYFNSIARQKILLADKAYQQKLKAA |
| | INSQEKSENTLKELEQKQCIEEDSEKKQALKERLIKTKNAYEKAKEKVSNLRKSRNKDSFQEYKN |
| | IIGQVEQTHLRDIISSQFNLHSDTKDRLTMIAKQDFENDIAEVLSGDRSLRTYKKNNPLYIRGRN |
| | TVLYKEGNEFFIKWIKGIVFKCILGVKNQNKTELYKTLECVLAEIHKICDSSMNFNQQDKLILNL |
| | TLDMPDKSEHRKVPERIAGVDLGLKIPAYFAVNDVPYIRKSIGKIEDFLKVRISIQSQKRSLQRA |
| | LQSSKGGKGRKKKLKTLDQFKEKEKNYITTYNHFISKKIISLAVQYGVEQINLELLTLKETQKKL |
| | LLRNWSYYQLQQFIEYKANREGISIKYVDPFHTSQTCSKCGHYEDGQREKQDTFCCKSCGFKDNA |
| | DYNAARNIAASTRYITNKKESEYYQQNNNEIA |
| 224 | MVKLKHNEYASLEKKLQKAQKEKTSDDNLIADLEEKLAAAKREMTDQARAICKYATEMSTETFAY |
| | KLATEIETNVFGQILSYLKKAVQSNFSNDAQAVRRGERSIRNYKKGMPIPFPLKDRIKIKDNDFY |
| | LHWYNNIRFKLHFGKDRSNNRQIVNRCFNSVNDYKLGSSASIQIVKRNGSPKFFLLLVINIPKEE |
| | VELNKKIVVGVDLGINVPAYVATNMTEERKAIGDREHFLNNRMAFQRRFKSLQRLRGTAGGKGRI |
| | KKLEPLENLRKTERNWVHTQNHLFSREVVKFAVQTHAATIHMEDLSNFGKDKDGNADEHKEFVLR |
| | NWSYYELQEMIRYKAKKYGIEVRNVRPAYTSQTCSWCGEQGFRQGATFICKNPECKQYSEKIHAD |
| | YNAARNIAKSKDIIKKNE |
| 225 | LYKAANNISSKLYLDEHVSSLVRLKHKEYKDLMRELAKAKKQKNLDEASIIEMEGRLRSYEEEMT |
| | DQELAICKYADEMSSLSLAYGFATELELDIYAQILTQIQSKVHQDFQNDQKDVREGKRSIRTYKK |
| | GMPIPFPWNNSIRIEISKKNKVEEQEKKTRRDSEDYDFYLNWYNGLRFRLHFGKDRSNNYQIIKR |
| | CFKLDEYCHDNYQLKASSIQLVIKNKKPELYLLLVVDIPQEKYSLNNKVVVGVDLGINTPAYVAT |
| | NVTEDRKAISDREHFLNARMAIKRRYRSLQRLKGTAEGRGRKKKLEPLERLREAESNWVHTQNHL |
| | FSRDIIKFALRVKAATIQMEKLEGFGKDEDGNVEEDKKFLLGEWSYYELQNMVKYKAAKVGIKVH |
| | FVKPAYTSQTCSWCGERGIRDGTAFYCQNPNCKQHGKKDINADYNAARNIAKSTEIVK |
| 226 | MRIIRPYYGDEIEKFITAGKEKSKADGADGALTKIFWDRLKASHPEIVSAGEFYGLLCAMRMEAV |
| | VYYNRAISKLYQSLIVDVDGAQVSTAKALSAGPYHEFRERFTSYISLGLRQKLQSNFRRKELLRC |
| | QLALPTAKSDRFPIPISKQVDKQGKGGFKVSELQNGDFIIELPLMAYHKAKGKTEREYVELDAGP |
| | AILNIPVILSTQRRRANKTWFKDEGTDAEIRRVMSGEYKVSWLEILQRNRPGKAHGDWYVNFTIK |
| | YQPKDCGLDPKVKGGIDIGLSSPLVCAVINSLDRLTIRDNDLVAFNKRALARRRTLLRRNRYKRA |
| | GHGSANKLEPITALTAKNELYRKAIMRRWAREAADFFRHNKAAAVNMEDLTGIKDREDYFSQMLR |
| | SYWNYSQMQTVLENKLKEYGIAVNYINPKDTSKTCHSCGHVNQHFDFSYRSANKFPMFKCVKCGV |
| | ECGADYNAARNIAVA |
| 227 | MIIARKIRLTVISDNRDEAYSFLRNEMYYYYKALNFAMNHVYFNYVAKEKIKMADDKYIEREQRY |
| | INAINNTHATLKKVRTESQKDLALKRIELNEQNLKKLRQSTSKEAREMLSQAISSAERTNTSDAV |
| | QKAFPMLTRDSIDFAASKATTEFNNDLKLGLLSGERVLRTYKKTNPLQIRGRLLKFFKEDGDYCI |
| | KFAKGIIFKCVLGIKRKNNTELAHTLEKVIDGTYKVCDSSFEYNDSKLILNLALQIDITARQQQP |
| | KVQGRVVGVDLGLKIPAFCALNDSIYIRERIGDIEDFLKVRTQLQARRKRLQRALKSAKGGKGQG |
| | KKLKALERFREHEKNFVKTYNHYLSKKIVHFSVLYGAEQINLELLQMAESQNKSILRNWSYYQLQ |
| | QMIEYKAAKEGIKIKYVDPYRTSQTCSKCGNYENGQRKTQATFECKSCKFKENADFNAARNIAKS |
| | TAYITDKFQSEYYKNYMDCKLGARASGFH |
| 228 | MISTRKIKVRCDDNTFYTFFRQEQREQNKALNIGIGIIHSNAILHNIDSGAEKKLKKSIEGLQGK |
| | IDKFNKHLEKEKLTDKKKEEVLKAIETTKKILDGEKKAFKKSEEYRKGIDELFKSTYLKSNTLDH |
| | VLDSMVNIQYKRTLSLVTQRIKKDYSNDFVEIITGQKSLRNYRNDNPLMISNQQLDFKYIEDTFY |
| | LDVMCGYRLEIILGRRDNQNVNELKSTLHRILSKEYKVCDSSMQFDKNNKDVILNLVIDIPNKSN |
| | MYEAIKERTLGIDLGMEVPIFMCLNDNTYIKKGIGDINNFLRVRQQIQVRRRKLQKDLTLTKGGK |
| | GRKKKLQLLDKLQENEKNFVKTYSHALSKRIILEFAKKNKCEYINLEKLTKDGFDNILRNWSYFD |
| | LQRMIENKAKREGIVVRYVNPSFTSQKCSKCGEIDKKNRQTQADFKCINCGFELNADHNAAINIA |
| | RSIDFV |
| 229 | MIITRKIAITIVSEEAQESYNYLRQQMYYYYKALNFGMNHIYFNYVAKEKIKLADSAYKEREEKY |
| | INAIHIAKEKLQKDLSVSQRAQAEKSLEVNTNNLDKLRKAISKDAKETFQKVMGAVERTNVTDAI |
| | KKEFPILQRDSIDFAASKVASDENNDLKLGLMTGSRTLRIYKRNQAYPFRSRRLKLYKENGDFFI |
| | KSSKSLLFKCLLGVKRQNSKELIQILEKILDNQYKICDSSLEFNKKKLILNLCIEVDENTHSENM |
| | KVPGVVKGRIVGVDLGIQIPAYCTLNDSPFKKKAIGSVDDLLRIRTQMQARRRRLSKNLISARGG |
| | KGRGKKLKALDRFEEYERNYVTTYNHFISKQIIQFTLQNQAEQINLELLQMEHTKSKSILRNWSY |
| | YQLQQMIEYKAKREGIVIKYVDPYHTSQVCSKCGHFEENQRMDQNTFRCKKCKYRTNADYNAAKN |
| | IANSTRYISSIQESEYYNIKNRNIVK |
| 230 | MELKRTARVKLAIPDDRRDDLKRTMLTFREVAQRFADRGWERDEDGYVITSRTRLQSLVYKQVRE |
| | DTGLHSDLCIGAVNLAADSLRSAVERMKAGKNVGKPTFTVPTATYNTGAVSYFTDGDGTGYCTLA |
| | AYGGRVRAEFVYPPDEDCPQRQYLGGDEWEPKGATLHYERDDGEYYLHVTVERDEPETELGEAEN |
| | GTVLGVDLGVENIAVTSAGAFYSGGLFNHRRDEYERIRGSLQQTGTESAHRTIEKMGDRERRWNT |
| | DVLHRISKAIVQEAITHDCSHIAFEDLTDIRDRMPGAKKFHGWAFRQLYEYVEYKAAEFGIATTQ |
| | VDPAYTSQRCSKCGTTLRENRTSQAAFCCQKCGYEVHADYNAAKNVATKLLRSGQKSPAGGATNQ |
| | LALKSGTLNGNGDFTPASS |
| 231 | MKEQLNKTLTFGLGKPLGWDIRGKAFIDPTEDQRREIYISLRATSRISAQMVNMLNAREYVRRIM |
| | KIPEAFVNEFKGSYIPIKQELKTLGLEEVDDISGATLSQTWALGVKPDFAGEHGKRLLMKGDRQL |
| | PTHRIDGTHPIYGRADGTKIILHEDRYFLIVQLFSSKWANKNEFPSGWIAFPVKIKPRDKTLAGQ |
| | FKRIIDGEWKLKNSHILRNPRKRGNTWLGQVVVSYTPDPFKDIDPKIIMGIDLGVSVPACLHIRE |
| | NGKAKKWAMQVGRGRDMLNTRGIIRSEIVRIIRSLRSKDSPLDNESKRAAKAKLKNLRKREKRVM |
| | KTASQKIAASIADVARRNGAGTWKMELLSENIKDEDPWLRRNWAPRMVVDAVRWQAEQVGAKLEF |
| | VDPAYTSQRCSKCGHISRENRPKGKKGAAHFECVRCGYKDHADKNAARNISTPGIVDLIKEQISK |
| | SPNGEER |
| 232 | MCRVLRRTVCLKLDVPDDRRDDLHETTDRFRQAAQIAVDRAFERNDDGYVITHKTKLHHLTYEQA |
| | REATDGLNANLVQAARNLAGDAAKGVVSRWENGKRASKPEFTAPTVVYNKKALTYREDGVSLATV |
| | NGRVECDFVLPPEGENPQTEYLRGDEWELRESTLHYRSESGEYYLHTTVAKDEDVSEEAENGTVL |
| | GVDVGVENIAATSTGRFWSSGLLNHRREQYESVRAGLQQTGTESAHRTIQQLGEREQRWVDDLLH |
| | RISKDIVSEAVEHDCSRIAFEDLTDIRERMPGAKKFHAWAFRRLFDLVSYKADERGIRTVQVDPA |
| | YTSQRCSKCGHTARNNRPSQAEFRCGRCGYENHADYNAAKNVAMKHVRAGQKSPRGRANRHLALK |
| | TGTLNGNGEFSPADA |
| 233 | MVITRKIELHLVHTGLSDEEYDQQWKFLHSINDNLYRAANRLVNQLYLNDEIDILLRYNNKEYME |
| | LRKQLAKKNLDKPVRAELKEKEKLVLEEIKAHRSAIFQRPYASVAYSMVTSQNEANIMTKILDVL |
| | KQDVLSHYSTNAKEVARGERSISNYRYGMPIPFAFDRTEKASICIYEENKKYFLKWYNNLRFELS |
| | FGRDRSNNQLVVQRCLGISNDGAKYKACNSSIQMVRKNGSTRLFLLLCVDVPKEINKHIKGKVVG |
| | IDLGLNVPIYAAVNDGPERKSIGSREAFLDQRASFQRRFRSLQKLQMTKGGHGRLHKLEPLERVR |
| | EAERNWVKNQNHLFSKEVVEFAKKVEAEIIQMERLKNFGRDDNEEIKEDKKYVVRNWSYFELQSM |
| | IEYKAKRAGIIVQYVNPAYTSQTCSECGQKGIRDNIHFKCLNPECNCFGKDIHADYNGARNIAKS |
| | KEIVKD |
| 234 | MKIKRTIKLVVKPSEKEKHILFKTFEEYKFAYNFVAEIGWKSNIHNSVKLHNLTYIVVREKTSLP |
| | SQLVISARNVASESLKSAFSRKKKGLAVSCPYSKNPAIRYDKRSYSVWFDREEISIATIEGRLKL |
| | KIKIPEYFRQYFSWDIRSASLKYNKRLKKFFFNIVVEKEIEEIPENDTVVGVDLGLSKLATLSTA |
| | DGKINKFFDGGHIRAVSERYFAIRKKLQSKGTPSAKRHLRKLSQKEKRFRTAINHKIAKEIVNLI |
| | PAGGTIVLEELKGIRERIRVYKRERRWVHSWNFAQLKQFIEYKAKSKGIKVVYINPKYTSQRCSK |
| | CGYVSKSNRKDQSHFKCSYCGYTVNADLNASRNIAINYLVSQKERLGHRVASLPVWAVVNQPNVR |
| | RLAVSSHS |
| 235 | MQRTIRIQLKPDIETDSVLSQTIEQYTWSFNAVCKHGWKNDLANGVELHKATYYDHRAITGLPSQ |
| | LVCAARVKATEALKSAKSLKKKGKTVSCPISKRCPIRYDARSYTAWFDRSELSILSINGRVKLSF |
| | EIAEYYRQYLAWKNTSADLLQDRKGCWWLHVVMEIETPQASVTDEVVGVDLGIASPAVDSRGSKY |
| | GSGHWKKIEDKTFELHRRLQSKGTKSAKGHLKKLSGRQRRFRKDCDHVLSKRLARSVESGATLVF |
| | EDLTNIRGRAKMRKAQRRRLHGWSFAQFQAFVTYKAEARGVNVGFVDPRYTSQKCSQCGHIERGN |
| | RPSQAEFRCKKCGYERHADYNAAINIRAEGIRMLKAEGSAVSAVGGEIRPKLGRKSKLRHSPVST |
| | EADTVLGTPSQCG |
| 236 | MPTIRRKIELLLDRTGLSKEEVDARWHTLHQINNNLYRTANNLINKLYLTDEIDDILRLNNQEYI |
| | DLKKQLGKKGLDETTKAELEERMHKIYATMNQHRSEILQRPRQSFAYSAVTGGDDTEIFNAKILD |
| | TLKQNVLAHYNADMKEVRRGEKSISNYKKGMPIPFSFDKSVRLYERNGQFFLKWYRDIQFILFFG |
| | HDASNNQLIVERCLGISEDGIAYKMCSSSLQMKGKKIFLLLVVDVPKEPFEQQKGMVVGIDLGLN |
| | VPIYATTNLTPERRAIGNRESFLNQRIAFQKRYKALQRLQLTKGGRGRSHKLEPLERLRETERNW |
| | VRTQNHLFSKEVIEFAKQVGASTINMERLASFGKNMSGEVYEDKKFVLRNWSYYELQNLIEYKAK |
| | RANIKVRYVNPAFTSQTCSECGQTGERDSIHFKCTNPECKNFGKSIHADYNGAKNIAKSTNIIKE |
| 237 | MVITRKIELWIAEEDKTKRNETWDFLRMLDKEIFRAANVVVNNQYFNDFYEERIIQQDEKIGDVS |
| | KKIRLLYSKLRKANDEEQQSLQNEIEILKEQKKESQTRVRSEFYKTSKQNTTYQILTKQFPEIPS |
| | DILTCLNNQIYSVIGKEKKEVLQGKRSIRSYRKGMPIPFRFAQHPRLEFFENEYYLRWLNNISFV |
| | LRFGRDKSNNRAILEKIFSKEYKLCDSSIQIDDRKIFLLLVVDIPKSEHNLNKELSVGVDLGLNV |
| | PAYCALSEGFARLAIGHKDDFVRVRQQMQRRRKALQKSLVLTSGGKGRTRKLKALDALGEKERHF |
| | VRTYNHTVAKRIVEFAEKYNAGVITMELLEGYGKDENGKSRKGDFVLRNWSYFELQTLLKDKAGR |
| | KGMDVVFIDPYHTSQTCALCGHYEIGQRETQANFICKNPVCKNFDEKVNADYNAALNIARSKKFV |
| | AKKEECEYFKLHKETRS |
| 238 | MKRTVKIKLSMNERKALEETIKQFKRACQMTVEEGWNENGLNNYKKYKLQKRVYDEIRETTDLQA |
| | NLVVRAIARGAQAVKGCTQLFENGFKASKPNFTSDSIAYDKRTLSVYPDEKRCTISTVNGRIDAN |
| | FVLPEEKNDYYKEYLDGSWEITQSTIEKHEYEKENSFYLHLGLEKEDEEIEHEDPTVMGVDLGMN |
| | NLAVTSTGKFFKGNQLDHNRKRFEEMRGKLQQKGTRSAHLTIQRMSERENRYACDTLHCISKELV |
| | EEAERNDVDIITFENLKYIRERMPKNKWYHVWAFNKLYQYVEYKAKERGIQVKQIDPRNTSRRCS |
| | KCGHTEKNNRNGNKFKCKQCGYELDADYNAAKNIGIKLLHRRQKSSAGAGNGQLALKSGTLKLNG |
| | EYSPTFLSARLCLRYFGIQKEF |
| 239 | MNTIKNTYIRTLKFNLNLTPKFETEEDNKKYINDVYSYLRDAIWAQNRAMNIVLDRTKEAYTLGR |
| | GMNRVKEIYYSYSHQKPISNDKKESFLESLLQYAPIDDQFVKNEVKKLRKFYESKKKPPKEETVS |
| | KNCEALKNKYIKYVGKSKDDVKRELDLLENYCAYPEDIYEKFANGLSTPAYIKQKVESYWKQDGI |
| | KTKVIYSMDENLRRIKDAPLFIPPNVFYNKKDELIGLVYDYTDYISFLEDLENKRNVNIYLSIPY |
| | KKGEDKLKFKLVLGNPHKSRDSRLSIKRIFEEEYRIKGSSIGFTKNKETGKNTNLTLYLTVEVPQ |
| | NKDNTLDENVVVGVDVGIAIPCVCALNNDKYTRENIGSYDTLFAKRTQFKMQRSRLNSQLKLSKG |
| | GHGRKRKLKKLELLSGKEKNYVDTECRKYASDVIKFCLKHHAKYINLEHLKGYRENPKVLAGWSF |
| | YKIQTYIEQAAEKHGIIVRKINPCYTSQICSVCGNWHPENRPKGKLGQAYFNCHNIDCKTHNTDL |
| | YKYGINADFNAARNIAMSTLFITDSDEITKKHWKEAREYYGIDESDDKEEKLNKVA |
| 240 | MTKVVKLALINNVTDKNGNKVEYYDLNKCLWDLQKETRDLKNTVIRECWEWYGFSNDYYKLNEEY |
| | PAERDHLKREKANGTIKDYSLDGFIYSKCSKKYKLHSGNLCTTLRAASGAFKTSLKDLLRGDKSV |
| | LSYKADQPLDVQKKCIVLEYDKDTNTYYITLILLNKAGVKCYNISDFRFKITVKDNSTRTILERC |
| | YDEVYSISSSKLIWNKKKGQWFLNLCYSFDKTETKELDKNKILGVNLGVYYPIYASISGEKDRLA |
| | ISGDELIEFRNRIEARRNALKKQAAVCGDGRIGHGYKTRMKPVLNISDKIANFRDTFNHKASRKL |
| | IDFAVKNDCGIIQLENLKGVTKDTEGFLKNWSFYDLQSKIENKAKERGIKVVYIEPAYTSLRCSK |
| | CGYIHKDNHPTREQFICQECGYRTLHDYNASQNIAVKDIDKIIKAELEKMGIKKNKDEEKPEK |
| 241 | MTKVVKLALINNVTDKNGNKVEYYDLNKCLWDLQKETRDLKNTVIRECWEWYGFSNDYYKLNEEY |
| | PAERDHLKREKANGTIKDYSLDGFIYSKYSKKYKLHSGNLCTTLRTASGAFRTSLKDILRGDKSV |
| | LSYKADQPLDVQKKCIVLEYDKDTNTYYITLTLLNKTGVKFYDIGDFREKVTVKDNSTRTILERC |
| | YDEIYSISASKLIWNKKKGQWFLNLCYSFDKTETKELDKNKILGVNLGVYYPIYASISGEKDRLA |
| | ISGDELIEFRNRIEARRNALKKQAAVCGDGRIGHGYKTRMKPVLNISDKIANFRDTFNHKASRKL |
| | IDFAVKNDCGIIQLENLKGVTKDTEGELKNWSFYDLQSKIENKAKERGIKVVYIEPAYTSLRCSK |
| | CGYIHKDNHPTREQFICQECGYRTLHDYNASQNIAVKDIDKIIKAELEKMGIKKNKDEEKPEK |
| 242 | MTTKTYAIKLIKPVDDSWDFAGETLRNLEYIVKRFKNKAATDQYLSIVSKDKKSAAEINTGVKQG |
| | LAELAYLNYAQECFNGAIKEAVDKVSKDFKGIVTGKSSLITYKDGQPIPVRSRQITLENDNGTYY |
| | ASIGLLSREYATELGREGRQKARIKFVLSSKGNEKVVLDRILSGEYKLCDSSIQRKGNAWYLQLA |
| | HSFEAKAKTDLIANRVLGIDIGISKAVYMAVSDSPVNAFIDGGEIEQFRNKTEHRRNQMRNQLKW |
| | CSDNRKSHGRNTLLKPLEVLESKVSDFRKLINHRYAKYVVDFAVKNQCSIIQMEDLSGINTRSAF |
| | LKRWSYFDLQTKIEDKAAAHGIKVVKVNPKYTSQRCYNCGVIAKDNRESQSVYKCECTRRTKKGV |
| | VAYKVNADLNAARNLSVLGIDKEIKAQCKAQKIAY |
| 243 | MITVRKLKVRCEDKSFYDFLRLEQREQNKALNLAIGYIHTSNILKSNDSGAETKIIKSISKLEDK |
| | IKKLNDELNKEKITDTKREKTLKAIETTTKILEGEKKILEEGKEFRIGLDKKFNEIYIDKNNMYH |
| | VLKTQTNVQYMRTLDLVKQKVSADYSNNFIDIVTGKISLMNYKQDFPLMIDNKNINLFKENDKYY |
| | IGIMLGYELEIVLGQRVNENILELKSILDKIIEDEEMAKQNKDHVKQYRFNQSSIQFDKNNNVIF |
| | NLTFTIPQDKTFKPVEGRVLGVDLGVKYPAYMCLSDDTYKREHIGSINDFLKVRTQMQNRRRELS |
| | KALSLTNSGKGRNKKTQALKRLSEKERNFAKTYNHAISKRIVDFAKKHKCEYIHLEKLTKDGFND |
| | RILRNWSYYELQRMVEYKADRIGIKVKYINPSYTSQKCSKCGHIDKENRQTQEKFVCTQCDFELN |
| | ADHNAAINIARATE |
| 244 | MITVRKLKLTIVGDEETRNQQYKLIRDEQYQQYRALNLCMSLLSTYNILNNWNSGAENKLNSQIE |
| | KLNKKVEKNKNDLKKDNLKENRIKKINESIKTLAKEKEKLQQEYLSSSEYRSDIDKKVKEMYIDD |
| | LYTVVQSQVNFKSKDMMSLVTQRSKKDFTTALKNGMAKGERSLTNYKRDFPLMTRGERWLKFEYD |
| | EESDDIYINWLHGIRFKVVLGYKKNENSIELRHTLHKVINKEYKICDSSMQFDRNNNLILNLTLD |
| | IPLNAKNEHIEGRTLGVDLGIKYPAYVCLSDDTYKRKSIGCAEDFIKFREQIRSRRYRLQKQLSM |
| | VKGGKGRNKKLQALDRIKDKERNFVKTYNHMISKNIVEFAKNHKCESINLEKLTKDGFPNMILSK |
| | WSYYELQNMIEYKAEREGISVKYVDPAYTSQTCSKCGYVDKENRTSQEKFKCIECGFELNADHNA |
| | AINIARSNNYVK |
| 245 | MANKNVNDSKIKTHILTKKVQLIVDTDREDTDEEKKAEVDRVYKYLRDSMKCQSREMNQYYMHLW |
| | MMSVARNLNDDRYSMKKYMDNIDAVHPYLDKNNKEFNNKQKKITRDFEKKIKNLCEEYQAETEIL |
| | NNDTLRELNKCFNRKKDGAYDTNFVNEMPEGLGIVMGRTVEQDFQNDCKAGLLSGIRNPRSYKIN |
| | YPLIIPKSFVAYGVGSGKAMQGRGIIFPDMEYSDFHNMLFSTKNPNITYNFVHDIDFKLVFGSMK |
| | RSHELRVIFDRIKMGEYSICGSTIEINNKKKIMLNLSYEAPIYEKPSLDENTVVGVDLGMAIPAV |
| | CSLNNDDRTYKYIGDSHELEFIKKGIQAQRRSYQRNAVYNKGGHGRNRKLENLDRLKKRERNTTR |
| | THNQRYAKQIVDFALANNAKYINLENLKGFSNNDKNKLVLRNWCYYELQQYIEIDASKYGIKVRY |
| | IYPMNTSRTCSVCGTLTTDEDVKNGVGRVSQDEFICKDPNCPSHTLYTKGPKGHKVPYFNADRNA |
| | SRNIAMSEDFVKKNAKDNAFKKIDDLYEINNDIDAA |
| 246 | MITVRKLKLTIVGDEEIRKEQYKFIRDSQYAQYQGLNLAMGVLTSSYLLSGGDVKSDYFKDAQKS |
| | LKNSNKIFNEINFGKGIDSKSYITKQVKKDFSTSLKNGLAKGERGFTNYKRDFPLMTRGRDLKFY |
| | EEDKEFYIKWVNKIVFKILTGRKDKNKVELIHTLNKVLNKEYKVSQSSLQFNKNNNLILNLTIDV |
| | KSDVKVEVIKDRVCGVGVGINTPIYVALNDILYISQSIGSNDELIKQKKQFEARKKRIRERVKNK |
| | KELNSLKEKERNWINTYNHMLSKRVVEFAKKNKCEYIYLEKINDNEFKNKVLKKWPYCELQKMIR |
| | YKAAGFGIEVKYIHSYHIFQKCSRCGYEYNKSIAIQKRFKCLNCGLEVNSDYNIARNISKYDILK |
| | DKSRITGNLVSE |
| 247 | MLNNKFIKDEKKLQESLNKLYMEKKESETKIKRSKIDEEIKKKVNVLKKMRDKESKEATKILQQA |
| | IKINLSNTTREIINQQFNLISDTKDRITQKVSQDFKADIKNGLLRGERVLRTYKKNSPLLIRGRT |
| | LQFYRKGNDILIKWYGGITFKCIIGKRKNNNHELYILLNKILENVCKVCDSSITIGRKLILNLSV |
| | ALTGFEADAPTVKGRVLGVNFGIKVPIYMSLNDKSYVQKSVGNLNDLLKLRVQLYKRKKKLENLI |
| | TNAIGDKVEKLKALNRLKEKEKNMLTTYNHYLSYNIVRFAKENQVGQINIEYLPFVKAKNKALKS |
| | WPYYQLQQFIEYKAKQKKIEVKYINSYLINEKCSNCGKNITHQLNSTNVFNCKKCEYKAHLDFNI |
| | SQNIALSTEYISIRK |
| 248 | MIITKKIKIIIIGENKDKYNKFIREEYYNQNKALNVAMNHLYFLHVAEEKIRMLNNKFIKDEKKL |
| | QESLNKLYMEKKESETKIKRSKIDEEIKKKVNVLKKMRDKESKEATKILQQAIKINLSNTTREIN |
| | QQFNLISDTKDRITQKVSQDFKADIKNGLLRGERVLRTYKKNSPLLIRGRTLQFYRKGNDILIKW |
| | YGGITFKCHIGKRKNNNHELYILLNKILENVCKVCDSSITIGRKLILNLSVALTGFEADAPTVKG |
| | RVLGVNFGIKVPIYMSLNDKSYVQKSVGNLNDLLKLRVQLYKRKKKLENLITNAIGDKVEKLKAL |
| | NRLKEKEKNMLTTYNHYLSYNIVRFAKENQVGQINIEYLPFVKAKNKALKSWPYYQLQQFIEYKA |
| | KQKKIEVKYINSYLINEKCSNCGKNITHQLNSTNVFNCKKCEYKAHLDFNISQNIALSTEYISIR |
| | K |
| 249 | MIITKKIKIIIIGENKDKHNKFIREEHYNQNKALNAAMNHLYFLHVAEEKIRMLNNKFIQDEKKL |
| | QESLNKLYTEKKESKTKIKRSEIDEKIKKKVNSLKKMRDKESKEAERILQQAIKINLSNTTREII |
| | NQQFNLISDTKDRITQKVSQDFKTDIKNGLLRGDRVLRTYKKTNPLLIRGRTLQFYRKGNDILIK |
| | WYGGVTFKCIIGQRKNNNHELYILLNKILENDSKVCDSSITIGRKLILNLSVALTGFEEDIPTVK |
| | GRVLGVNFGMKVPIYMSLNDKPHVQKSVGNLNDLLKLRVQLYKRKKKMKNLIIKSIGDKAEKLKV |
| | LNRFKEKEKNILTTYNHYLSYNIVQFAKENQVGQINIEYLPLVKTKNKALKSWPYYQLQQFIEYK |
| | AKRKKIEVKYINAYLLNKKCSNCGKDTTHQSNSNNIFNCKKCQYRAPLDSNISRNIALCTEYISI |
| | RKE |
| 250 | MKLKRTIKLVVKPSEEEKQILFKTLEEYKFAYNFVAEIGWKSKVSNSIKLHNLTYTTVREKTSLP |
| | SQLVISARMVASESLKSAFNRRKKGLKVSCPYSNNPAIRYDKRSYSVWFDREEISIATVEGRLKL |
| | KIKIPEYFKQYLNWKIRSASLKYDKRLKKFFFNIVVEKEIEEIPENDTVIGVDLGLSKLAVISTA |
| | DGKINKFFDGRHIRAVSERYFAIRKKLQSKGTPSAKRHLKKLSQKEKRFRTAINHKIAKEIVSLV |
| | PAGGTIVLEELKGIRERIKVSKKERRWIHSWNFAQLQQFIEYKAQSKGIKVVYINPKYTSQRCNK |
| | CGHISKSNRKDQSHFKCSSCGYTINADLNASRNIAINYLVSQQERLGHRVASLPVWAVVNQPNVR |
| | GLANFSHS |
| 471 | MAKNTITKTLKLRIVRPYNSAEVEKIVADEKNNREKIALEKNKDKVKEACSKHLKVAAYCTTQVE |
| | RNACLFCKARKLDDKFYQKLRGQFPDAVFWQEISEIFRQLQKQAAEIYNQSLIELYYEIFIKGKG |
| | IANASSVEHYLSDVCYTRAAELFKNAAIASGLRSKIKSNFRLKELKNMKSGLPTTKSDNFPIPLV |
| | KQKGGQYTGFEISNHNSDFIIKIPFGRWQVKKEIDKYRPWEKFDFEQVQKSPKPISLLLSTQRRK |
| | RNKGWSKDEGTEAEIKKVMNGDYQTSYIEVKRGSKIGEKSAWMLNLSIDVPKIDKGVDPSIIGGI |
| | DVGVKSPLVCAINNAFSRYSISDNDLFHENKKMFARRRILLKKNRHKRAGHGAKNKLKPITILTE |
| | KSERFRKKLIERWACEIADFFIKNKVGTVQMENLESMKRKEDSYFNIRLRGFWPYAEMQNKIEFK |
| | LKQYGIEIRKVAPNNTSKTCSKCGHLNNYFNFEYRKKNKFPHFKCEKCNFKENADYNAALNISNP |
| | KLKSTKEEP (Un1Cas12f1) |

TABLE 5

| SEQ | | Corresponding |
|---|---|---|
| ID | | nuclease |
| NO | sgRNA sequence | SEQ ID NO |
| 251 | AATTGCGAGTATAAAGCAAACACAGTTATAGTGTGGTATTCGCAATTAATTTCGGGCGAC | 1 |
| | TCGGCGTCCGTGAATCGAGAAAGTATATGTGAGTCTGAATCATAATCAGCAATAGATACA | |
| | CTCGATAAGGTGAAAACAATACACATTTAATCCGTGTATTCAACTAATCCTTGTGTATATT | |
| | TGACGAAAGTTGCAACCTATACACTCGTGAGAGTTGCGAGA | |
| 252 | ATTCGCAATTAATTTCGGGCGACTCGGCGTCCGTGAATCGAGAAAGTATATGTGAGTCTG | 1 |
| | AATCATAATCAGCAATAGATACACTCGATAAGGTGAAAACAATACACATTTAATCCGTGT | |
| | ATTCAACTAATCCTTGTGTATATTTGACGAAAGTTGCAACCTATACACTCGTGAGAGTTGC | |
| | GAGA | |
| 253 | ATTCGCAATTAATTTCGGGCGACTCGGCGTCCGTGAATCGAGAAAGTATATGTGAGTCTG | 1 |
| | AATCATAATCAGCAATAGATACACTCGATAAGGTGAAAACAATACACATTTAATCCGTGT | |
| | ATTCGAAAGCGAGA | |
| 254 | AATTGCGAGTATAAAGCAAACACAGTTATAGTGTGGTATTCGCAATTAATTTCGGGCGAC | 1 |
| | TCGGCGTCCGTGAATCGAGAAAGTATATGTGAGTCTGAATCATAATCAGCAATAGATACA | |
| | CTCGATAAGGTGAAAACAATACACATTTAATCGAAAGTTGCAACCTATACACTTGTGAGA | |
| | GTTGCGAGA | |
| 255 | ATTCGCAATTAATTTCGGGCGACTCGGCGTCCGTGAATCGAGAAAGTATATGTGAGTCTG | 1 |
| | AATCATAATCAGCAATAGATACACTCGATAAGGTGAAAACAATACACATTTAATCGAAA | |
| | GTTGCAACCTATACACTTGTGAGAGTTGCGAGA | |
| 256 | ATTCGCAATTAATTTCGGGCGACTCGGCGTCCGTGAATCGAGAAAGTATATGTGAGTCTG | 1 |
| | AATCATAATCAGCAATAGATACAGAAAGCGAGA | |
| 257 | CATAATGATTCGCACTCTTTCGGGCGGCTCGGCGTCCGTAAACCGAGAAAGTATAAGTCA | 2 |
| | GTCTGAATTTCATTCAGCTTTAGATACACTCGGTAAGGTTCAAACAATACACATTCAATCC | |
| | GTGTATTCAGTCCGAAAGCAGCTGCAATCTGCATATAGCATGTGGACTGCGAG | |
| 258 | ATTCGCACTCTTTCGGGCGGCTCGGCGTCCGTAAACCGAGAAAGTATAAGTCAGTCTGAA | 2 |
| | TTTCATTCAGCTTTAGATACACTCGGTAAGGTTCAAACAATACACATTCAATCCGTGTATT | |
| | CAGTCCGAAAGCAGCTGCAATCTGCATATAGCATGTGGACTGCGAG | |
| 259 | ATTCGCACTCTTTCGGGCGGCTCGGCGTCCGTAAACCGAGAAAGTATAAGTCAGTCTGAA | 2 |
| | TTTCATTCAGCTTTAGATACACTCGGTAAGGTTCAAAGAAATGCGAGatGTCTTCGAGAAG | |
| | ACCT | |
| 260 | ATTTATTGGGCGCTTTCTCGCCCATAAAACGAGAAGTACCGCTCACAGTGGCGGCAACAC | 3 |
| | TCGTGAAGGTAGTCCCATCGTTTCGGGTGGGCTGAAATCTCAGTCACAAAAACCGACTGA | |
| | GGAACCCTTGCAACTACATATTTGGTAGATGTAAAgaaaGTTTaCATCTTACCTATAAGGGT | |
| | TTGAAACatGTCTTCGAGAAGACCT | |
| 261 | AACGAGAAGTACCGCTCACAGTGGCGGCAACACTCGTGAAGGTAGTCCCATCGTTTCGG | 3 |
| | GTGGGCTGAAATCTCAGTCACAAAAACCGACTGAGGAACCCTTGCAACTACATATTTGGT | |
| | AGATGTAAAgaaaGTTTaCATCTTACCTATAAGGGTTTGAAACatGTCTTCGAGAAGACCT | |
| 262 | AACGAGAAGTACCGCTCACAGTGGCGGCAACACTCGTGAAGGTAGTCCCATCGTTTCGG | 3 |
| | GTGGGCTGAAATCTCAGTCACAAAAACCGACTGAGGAACCCTTgaaaAAGGGTTatGTCTTC | |
| | GAGAAGACCT | |
| 263 | ACGCCACTGATGTGGCAGGTTCGCACCTAATTTcGGGGCGACTTCCCGCCCTGAAATCGA | 4 |
| | GAAAGTGGCCGTAAGACGCAGTTCTTTGCGCCGGCAATACACTCGAAAAGGTTAAGATG | |
| | CACATAGTAATCCGTGCATGGGTCATgaaaGTTGCAACACGCGCGTAAGGATGACTTGAAG | |
| | GatGTCTTCGAGAAGACCT | |
| 264 | GTTCGCACCTAATCTTGGGGCGACTTCCCGCCCTGAAATCGAGAAAGTGGCCGTAAGACG | 4 |
| | CAGTTCTTTGCGCCGGCAATACACTCGAAAAGGTTAAGATGCACATAGTAATCCGTGCAT | |
| | GGGTCATgaaaGTTGCAACACGCGCGTAAGGATGACTTGAAGGatGTCTTCGAGAAGACCT | |
| 265 | GTTCGCACCTAATCTTGGGGCGACTTCCCGCCCTGAAATCGAGAAAGTGGCCGTAAGACG | 4 |
| | CAGTTCTTTGCGCCGGCAATACACTCGAAAAGGTTAAGATGCACATAGTAATCCgaaaGGA | |
| | TGACTTGAatGTCTTCGAGAAGACCT | |
| 266 | TTTATAACACAGCAGTAACACCATAAACTAAATTAACTGTTTGTTACTGTCTGCGGGCGA | 5 |
| | TTTCACGTCCGAAATATGAGGGTGTAAAGAAATTTAAGTATTTGCAATATCCACTCATAA | |
| | AACCGTGCATCTACATAAGTTGCGAgaaaGTCGCGATTTGCGTAGGTGCATGGGATGAAAA | |
| | atGTCTTCGAGAAGACCT | |
| 267 | ATTAACTGTTTGTTACTGTCTGCGGGCGATTTCACGTCCGAAATATGAGGGTGTAAAGAA | 5 |
| | ATTTAAGTATTTGCAATATCCACTCATAAAACCGTGCATCTACATAAGTTGCGAgaaaGTCG | |
| | CGATTTGCGTAGGTGCATGGGATGAAAAatGTCTTCGAGAAGACCT | |
| 268 | ATTAACTGTTTGTTACTGTCTGCGGGCGATTTCACGTCCGAAATATGAGGGTGTAAAGAA | 5 |
| | ATTTAAGTATTTGCAATATCCACTCATAAAACCGTGCAgaaaTGCATGGGatGTCTTCGAGAA | |
| | GACCT | |
| 269 | ACATAGTTgTTCGGCTTTGTTCGCGTAAGTTgTCGGGGCGACTTCCCGTCCCTAAATCGAG | 6 |
| | AAAGTGGCCGTAAGTCTTCGAATTTCGAAGCCGACAATACACTCGAGAAGgaaaGTTGCAA | |
| | CCCGCGCGTATATGCGGCTTGAAGGatGTCTTCGAGAAGACCT | |
| 270 | GTTCGCGTAAGTTgTCGGGGCGACTTCCCGTCCCTAAATCGAGAAAGTGGCCGTAAGTCT | 6 |
| | TCGAATTTCGAAGCCGACAATACACTCGAGAAGgaaaGTTGCAACCCGCGCGTATATGCGG | |
| | CTTGAAGGatGTCTTCGAGAAGACCT | |
| 271 | GTTCGCGTAAGTTgTCGGGGCGACTTCCCGTCCCTAAATCGAGAAAGTGGCCGTAAGTCT | 6 |
| | TCGAATTTCGAAGCCGgaaaCGGCTTGAAGGatGTCTTCGAGAAGACCT | |
| 272 | GTGATATAAAAATACCGTAAGGTTCGCACTTTgTTCGGGCGACTCGTTCGTCCGTAAATCG | 7 |
| | AGAAAGTATGCGTAAGACCTGATTTATCGGGGGGCAGATACACTCGATAAGGTgaaaTGTT | |
| | GCAACTCGCACGAGGGTATGTACTGCGAGatGTCTTCGAGAAGACCT | |
| 273 | GTTCGCACTTTgTTCGGGCGACTCGTTCGTCCGTAAATCGAGAAAGTATGCGTAAGACCTG | 7 |
| | ATTTATCGGGCGGCAGATACACTCGATAAGGTgaaaTGTTGCAACTCGCACGAGGGTATGT | |
| | ACTGCGAGatGTCTTCGAGAAGACCT | |
| 274 | GTTCGTCCGTAAATCGAGAAAGTATGCGTAAGACCTGATTTATCGGGCGGCAGATACACT | 7 |
| | CGATAAGGTgaaaTGTTGCAACTCGCACGAGGGTATGTACTGCGAGatGTCTTCGAGAAGAC | |
| | CT | |
| 275 | ACATAATTgTAAATAATTATTCACACTTTCTCTTGGGCGGCTCGGCGTCCATAAATCGAGA | 8 |
| | AAGTATGGGTAAGTCTGAATTTATTCAGCACCAGATACACTCGGTAAGGTATAAACTATA | |
| | CACATTAAATCgaaaGATTCAATCAGCATGGACAGGTGTCCTGCGAGatGTCTTCGAGAAGA | |
| | CCT | |
| 276 | ATTCACACTTTCTCTTGGGCGGCTCGGCGTCCATAAATCGAGAAAGTATGGGTAAGTCTGA | 8 |
| | ATTTATTCAGCACCAGATACACTCGGTAAGGTATAAACTATACACATTAAATCgaaaGATTC | |
| | AATCAGCATGGACAGGTGTCCTGCGAGatGTCTTCGAGAAGACCT | |
| 277 | ATTCACACTTTCTcTTGGGCGGCTCGGCGTCCATAAATCGAGAAAGTATGGGTAAGTCTGA | 8 |
| | ATTTATTCAGCACCAGATACACTCGGTAAGGTATAAACTATACACATTAAATCgaaaGATTC | |
| | AATCAatGTCTTCGAGAAGACCT | |
| 278 | CTTCGCACCGTCTCTGGGGCGACTTCCCGTCCCAAAATCGAGACAGTGGCCGTCAGCCTT | 9 |
| | CCCATCGGGAAGCGGGCAATACACTCGAAAAGGTTAAGATGCACATAGTAATCCGTGCA | |
| | TGAGCCACACCgaaaGATGCATCTCACGCGTGTCCGTGGCTTGAAGGatGTCTTCGAGAAGA | |
| | CCT | |
| 279 | CTTCGCACCGTCTCTGGGGCGACTTCCCGTCCCAAAATCGAGACAGTGGCCGTCAGCCTT | 9 |
| | CCCATCGGGAAGCGGGCAATACACTCGAAAAGGTTAAGATGCACATAGTAATCCGTGCA | |
| | TGAGCCACACCgaaaGATGCATCTCAACCTTGTCCGTGACGGGAAGGatGTCTTCGAGAAGA | |
| | CCT | |
| 280 | CTTCGCACCGTCTCTGGGGCGACTTCCCGTCCCAAAATCGAGACAGTGGCCGTCAGCCTT | 9 |
| | CCCATCGGGAAGCGGGCAATACACTCGAAAAGGTTAAGATGCAgaaaTGCATCTCACGCGT | |
| | GTatGTCTTCGAGAAGACCT | |
| 281 | GAAGGGGCGACTTCCCGTCCCAAAATCGAGATAGTGGTCCTGATTCTTTGATTTCAAAGC | 10 |
| | GGACAATACACTCGATAAGGTTAAGATGCACATAGGAATCCGTGCATGGGTCACAATgaaa | |
| | GTTGCAACCCGCTCGCTGGTGTGACTTGAAGGatGTCTTCGAGAAGACCT | |
| 282 | GAAGGGGCGACTTCCCGTCCCAAAATCGAGATAGTGGTCCTGATTCTTTGATTTCAAAGC | 10 |
| | GGACAATACACTCGATAAGGTTAAGATGCACATAGGAATCCGTGCATGGGTCACAATgaaa | |
| | GTTGTAACCCGCTCGATGGTGTGACGGGAAGGatGTCTTCGAGAAGACCT | |
| 283 | GAAGGGGCGACTTCCCGTCCCAAAATCGAGATAGTGGTCCTGATTCTTTGATTTCAAAGC | 10 |
| | GGACAATACACTCGATAAGGTTAAGgaaaCTTAAAGGatGTCTTCGAGAAGACCT | |
| 284 | GTTTGCAACACGGCAAGGGTGACTCTACACCCCAAAATCGAGATCAGTACGGCAAAAAG | 11 |
| | AGCGCTTCTGTTCTGCCGGATACACTCGATAAGGTATAAATTGTATgaaaGATGCAATCCGC | |
| | GTGCGGGTGCGGCTGAGAGGatGTCTTCGAGAAGACCT | |
| 285 | AGGGTGACTCTACACCCCAAAATCGAGATCAGTACGGCAAAAAGAGCGCTTCTGTTCTGC | 11 |
| | CGGATACACTCGATAAGGTATAAATTGTATgaaaGATGCAATCCGatGTCTTCGAGAAGACC | |
| | T | |
| 286 | GTTTGCAACACGGCAAGGGTGACTCTACACCCCAAAATCGAGATCAGTACGGCAAAAAG | 11 |
| | AGCGCTTCTGTTCTGCCGgaaaCGGCTGAGAGGatGTCTTCGAGAAGACCT | |
| 287 | TTGGGGGCGACTTCCCGTCCCGAAATCGAGAAAGTGGCTGTAAGTCCCGTTCTTTaCGGG | 12 |
| | CAGGCAAGACACTCGAAAAGGTTAAGATATGCACATAGTAATCCGTGCATGAGCCACTG | |
| | TATTGTGCATTgTTGCAgaaaGTTGCAACTCATGCGTATGCGTGGCTTGAAGGatGTCTTCGA | |
| | GAAGACCT | |
| 288 | TTGGGGGCGACTTCCCGTCCCGAAATCGAGAAAGTGGCTGTAAGTCCCGTTCTTTaCGGG | 12 |
| | CAGGCAAGACACTCGAAAAGGTTAAGATATGCACATAGTAATCCGTGCATGAGCCACgaaa | |
| | GTGGCTTGAAGGatGTCTTCGAGAAGACCT | |
| 289 | ATCGAGAAAGTGGCTGTAAGTCCCGTTCTTTaCGGGCAGGCAAGACACTCGAAAAGGTTA | 12 |
| | AGATATGCACATAGTAATCCGTGCATGAGCCACgaaaGTGGCTTGAAGGatGTCTTCGAGAA | |
| | GACCT | |
| 290 | GTTCGGGGCGACTTCCCGTCCCAAAATCGAGAAAGTGGCTGTTAGCCCCGGATTATCCGG | 13 |
| | GCGGGCAATACACTCGAGAAGGTTAAGATGCACATAGTAATCCGTGCATGAGTCACTGT | |
| | GCTGTGCATATTTGCCGTTGCAACTTACACGTGTACGTGACTTGAAGGatGTCTTCGAGAA | |
| | GACCT | |
| 291 | GTTCGGGGCGACTTCCCGTCCCAAAATCGAGAAAGTGGCTGTTAGCCCCGGATTATCCGG | 13 |
| | GCGGGCAATACACTCGAGAAGGTTAAGATGCACATAGTAATCCGTGCATGAGTCACgaaaG | |
| | TGACTTGAAGGatGTCTTCGAGAAGACCT | |
| 292 | ATCGAGAAAGTGGCTGTTAGCCCCGGATTATCCGGGCGGGCAATACACTCGAGAAGGTT | 13 |
| | AAGATGCACATAGTAATCCGTGCATGAGTCACgaaaGTGACTTGAAGGatGTCTTCGAGAAG | |
| | ACCT | |
| 293 | CATCTCCTGTCGGGGGCGTCTTCCCGTCCCTAAATCGAGATAGCAGCCATTTgTCTTCATTa | 14 |
| | TTTGAAGACGGTCTTGCACTCGAAAAGGTCAAGATGCACACAATAATgaaaGTTGCAACTC | |
| | GCACGTTGGCACTGGTTGAAGGatGTCTTCGAGAAGACCT | |
| 294 | CATCTCCTGTCGGGGGCGTCTTCCCGTCCCTAAATCGAGATAGCAGCCATTTgTCTTCATTa | 14 |
| | TTTGAAGACGGTCTTGCACTCGAAAAGGTCAAGATGCACACAATAATgaaaGTTGtACTTGC | |
| | AtGTTGGCACTTGTTGAAGGatGTCTTCGAGAAGACCT | |
| 295 | CATCTCCTGTCGGGGGCGTCTTCCCGTCCCTAAATCGAGATAGCAGCCATTTgTCTTCATTa | 14 |
| | TTTGAAGACGGTCTTGCACTCGAAAAGgaaaCTtaTcGAAGGatGTCTTCGAGAAGACCT | |
| 296 | ATTCAGGGGCGACTTCCCGCCCTGAAATCGAGAAAGTGGTCGTAAGCCGGAAGCATTTCC | 15 |
| | GCAGACAATACACTCGAAAAGGTTAAGATATGCACATAGTAATgaaaGTTGCAACACGCGC | |
| | GAAGGTGCGGCTTGAAGGatGTCTTCGAGAAGACCT | |
| 297 | ATTCAGGGGCGACTTCCCGCCCTGAAATCGAGAAAGTGGTCGTAAGCCGGAAGCATTTCC | 15 |
| | GCAGACAATACACTCGAAAAGGTTAAGgaaaCTTGAAGGatGTCTTCGAGAAGACCT | |
| 298 | ATTCAGGGGCGACTTCCCGCCCTGAAATCGAGAAAaTtGTCGTCAGACAATACACTCGAA | 15 |
| | AAGGTCAAGgaaaCTTGAAGGatGTCTTCGAGAAGACCT | |
| 299 | ATTCGAGATAGTGGCAGTAAGCGCCTCCGCAGGGGGCTGTGCAATACACTCGAAAAGGT | 16 |
| | TAAGATGTACACATAGTAATCCGTGTACGACCAGCATTCTGTGCTTcTTGCCGCTGCAAGC | |
| | AGCATATGTACGCTGGTTGAAGGatGTCTTCGAGAAGACCT | |
| 300 | ATTCGAGATAGTGGCAGTAAGCGCCTCCGCAGGGGGCTGTGCAATACACTCGAAAAGGT | 16 |
| | TAAGATGTACACATAGTAATCCGTGTACGACCAGCgaaaGCTGGTTGAAGGatGTCTTCGAG | |
| | AAGACCT | |
| 301 | ATTCGAGATAGTGGCTGCAAGTGTGCAATACACTCGAAAAGGTTAAGATGTACACATAGT | 16 |
| | AATCCGTGTACGACCAGCgaaaGCTGGTTGAAGGatGTCTTCGAGAAGACCT | |
| 302 | GGTCGGATGTTTCCGGCAATACACTCGGTAAGGTAGCGCGAATGTGAAGTGTACATGAAA | 17 |
| | ATATAAAGAGATTCGCGCTTATTGAAATTACTAGCACAAATACCGCTAGTACAGTTTACA | |
| | CATCAAGCGAATTGCAACgaaaGTTGCAATTCGTGCGCATGTGTGAATTGCAAGatGTCTTC | |
| | GAGAAGACCT | |
| 303 | GGTCGGATGTTTCCGGCAATACACTCGGTAAGGTAGCGCGAATGTGAAGTGTACATGAAA | 17 |
| | ATATAAAGAGATTCGCGCTTATTGAAATTACTAGCACAAATACCGCTAGTACAGTTTACA | |
| | CATgaaaATGTGTGAATTGCAAGatGTCTTCGAGAAGACCT | |
| 304 | CTCGGTAGTAGCGCGAATGTGAAGTGTACATGAAAATATAAAGAGATTCGCGCTTATTGA | 17 |
| | AATTACTAGCACAAATACCGCTAGTACAGTTTACACATgaaaATGTGTGAATTGCAAGatGT | |
| | CTTCGAGAAGACCT | |
| 305 | CTTCGCACATATTTAGGGCGACTTCACGTCCTCAAATCGAGAAAGTGAGCGTAAGACTTG | 18 |
| | GCTTCTGTCAAGCGGTTAATACACTCGAGAAGGTTAATATGCACATAGTAATgaaaGTTGC | |
| | AATTTGTATACGAGTGTGACTTGAAGGatGTCTTCGAGAAGACCT | |
| 306 | TAGGGCGACTTCACGTCCTCAAATCGAGAAAGTGAGCGTACTTGGCTTCTGTCAAGCGGT | 18 |
| | TAATACACTCGAGAAGGTTAATATGCACATAGTAATgaaaaTTGCtATTTGcATACatGTCTTC | |
| | GAGAAGACCT | |
| 307 | TAGGGCGACTTCACGTCCTCAAATCGAGAAAGTGAGCGTACTTGGCTTCTGTCAAGCGGT | 18 |
| | TAATACACTCGAGAAGGTTAATATGCAgaaaTGcATACatGTCTTCGAGAAGACCT | |
| 308 | TAAGTGGATATCCAACgaaaTTTGATATAGGATGTATATACGAATTTCAATTACCACCCCAA | 19 |
| | TGGGGTGAGGGCGTGTTGGAGCGCCTTAGTTTGAGGTTTGATACTAAAAATTGAGATGAT | |
| | GGAGGTCATTTCGATAATCAAGCACTCAAAAAATCTACTTA | |
| 309 | CTTCGGGAATGGGCGTGTTGGAACGCCTTAGTTTGAGGTCAGGATTAAAAAATTGACAAG | 20 |
| | ACGCAGGTCTaTTCAGTACCGTGGCACTCAAAAAATTCACTTGATTaTaTCAAGTGAATATC | |
| | CAAC | |
| 310 | TTGAAATAAAATGAATTTCAAACCCCTTCGGGGGTGGGCGTGTTGGAGCGCCTTAATTTG | 21 |
| | AGGTGCAGAATCCAAAAACTGCGACGATGTAGGTCGTTTCAGTCTCTGCGCACTCAAAAA | |
| | ATTCACTTGATTaTTCAAGTGAATATCCAAC | |
| 311 | CTTTGATATAAAATAGATATGAATTTCATTGCCCATTaTGGGCTGGGCGTGTTGGAACGCC | 22 |
| | TTAGTTTGAGGTCTGAAAATGAAAATTGTGGTTGCATAGGCACTCTCGATATTCAAGaaagG | |
| | GTGTTAATGCCTTGAGTaTTAAGTG | |
| 312 | GGTGTTAATGCCTTGATATTTAAGTGAATATCCAATAATAGATATAATGGATTTCAAGTCC | 23 |
| | CTTCGGGGACGGGCGTGTTGGAACGCCTTAGTTTGAGGTTTGGATTC | |
| 313 | ATTAAACCCCATTATGGGGTGGGCGTGTTGGAACGCCTTAGTTTGAGGTTTGAAAAACAA | 24 |
| | ATTTGGGTTATATTTGGTAATCTTAATGTTCAAGCACTCAAAAAATTCACTTAAATTAaTTT | |
| | AAGTGGATATCCAAC | |
| 314 | CTTCGGGGACGGGCGTGTTGGAACGGCCTCAATTaTGAGGCTTAGCCTTAGTTTGAGGTTT | 25 |
| | GGATTCAAAAAATCGTTGGTGTGTAGGCACTTTCGATTTCCAAGCACTCAAAAAATTCAC | |
| | TTATAAGTGAATATCCAAC | |
| 315 | GTTCTTTGATATAAGTATTAGATATGAATTTCAATTCCCCTCTGGGGGAAGGGCGTGTTGG | 26 |
| | AACGCCTTAGTTTGAGGTTTGAAAATGAAAAATTGGGTGGTGTGGAGGCACTCCCAATaaa | |
| | gGATGTTATCGGATATCCAAC | |
| 316 | GTTCTTTGAGGTAAAATAGATATGAATTTCATTACCCATTaTGGGGTGGGCGTGTTGGAAC | 27 |
| | GCCTTAGTTTGAGGTTTGAAAACAGAAATTAGGATTGCGGAGGCATTCTTGATGTTCAAG | |
| | CAaaagAGTGTTAATGCTTTGAC | |
| 317 | ATTTCATTGCCTATTaTGGGCTGGGCGTGTTGGAACGCCTTAGTTTGAGGTTTGAAAACGA | 28 |
| | AAATTGGGAATGTAGAGGCACTCTCGATATTCAAGaaagCTTGAATT | |
| 318 | ATTTCTACACCCATTATGGGTTGGGCGTGTTGGAACGCCTTAGTTTGAGGTTTGAAGATAA | 29 |
| | AAATCAAATTGGTGGAGGCCTTTGATATTCAAGCACTCAAAAAATTCACTTATTTGTGAT | |
| | ATATAGTTGGAAATCAACACATAGTGGATATCCAAC | |
| 319 | GTTACGCATACGTCATTGCGAGGAGGACTTTAGTCCGACGTGGCAATCTCTaTTGAGGTGA | 30 |
| | CTCATATTTACTATAtAATaaggATTaTTAAGTGGATAT | |
| 320 | TTGAAATAAAATGAATTTCAAACCCCTTCGGGGGTGGGCGTGTTGGAGCGCCTTAGTTTG | 31 |
| | AGGTGCAGAATCCAAAAACTGCGACGATGTAGGTCGTTTCAGTCTCTGCGCACTCAAAAA | |
| | ATTCACTTGATTTCAAGTGAATAT | |
| 321 | CAACCTGCGTGGCCTGAAGGTGAGAAGTACATGCATTAATGGCGCTCGCGCCTTGCATGT | 32 |
| | GGCGCTCATCGATCGCTCGaaagCCCGCGCGGTACTCGAATTTGACGTGTGAGCGCAGG | |
| 322 | GTTGAGAAGGAATGTGCATATTAGCGGCGTTTCGCCCTTTGCACCCGCTCACCGAACACC | 33 |
| | ATCAGGCGGGTTTATATGGTTAATTCGACCTAAGATTCGTTTACCAAGaaagCTTGGATAAG | |
| | CCCGTTTGATGGGTGTATTCAGA | |
| 323 | GTTCTTTGAAATATATAGATATGGATTTCAATTTCCCGTTTATGGGATGGGCGTGTTGGAA | 34 |
| | CGCCTTAaaagGGTGTATTTGCCTATGTATTTAAGTGGATATCCAAC | |
| 324 | CTTAATCATACAGAAATGTATGCGGCTGATTTCGCAGCCGAAAGGTGAGGATTATTGATA | 35 |
| | TaTTTGAAATCCCATaaagGTTTGAGAGGTATATCAAATAACACAGGTACGAAAC | |
| 325 | CTTTCGGGATGGGCGCGTTGGAGCGCCTTGGTTTGAGGTGAGGACACCATAATCCGCATA | 36 |
| | ATGAATATTGTACGGATGTCCCTGCACTCGAAAAGTTCACTTGATTaTCAAGTGAATATCC | |
| | AAC | |
| 326 | CAAAGATATTTATTAGGGCATGTTGGAATGCCTAAGTTTGAGGTAGAAACAAAAAAAGC | 37 |
| | ATTTAAACAGAGGTTTAGTGTTGTCTTTGCACTCAAAAAATCCGTTCAAATATGGCTGTAaa | |
| | agGGTGTTACTGCACCTAGGGATATCCAAC | |
| 327 | GTTTeGGTGTGTTTATATGGCCTCAAATATAAACACCGCTATTGTGGATACAATAGTACGC | 38 |
| | CGAAAGGTGAGGATTCGTCACTCACTAAAATCCATGTTAAaaagATTTAACATGAG | |
| 328 | ATTCACTGTCCTAAGTCTGAGGCTAAATTGGCACTCGGAAAGGGTAAAGGCTTGACACTG | 39 |
| | TGTTACCGTCAAGACATTTCACATAAGTGAAATGTGAAT | |
| 329 | ATTAAGTATATTCGCACCATTTAAGGGGCGGCTCGGCGTCCCAAAATCGAGAAAGTATAT | 40 |
| | GTAAATCTGAATTaaagGTTGCAATTCGTTTGTACAGGTAAGTTGCGAG | |
| 330 | ACGTACTATATAGATTATAAATTTGCAGTGAGCAAGTTTAACCTATTACATAGGCAAAAT | 41 |
| | AATTGGTATTACTTAATTAGAAGTATATTAAAAAAATTATATGGAATCCTTATAGGAGGT | |
| | ACACTGCAAAATTTAATATTTAAAaaagGTTTAAACATTACTATGTAGTATGGGAAT | |
| 331 | GTTGGTTGCCCTTAGTTTGAGGTAGAAATCCAAAAAACGTGGCAGTTGTATCTGCTTCGT | 42 |
| | GGCTCTACACTCGAAAAATACCATCATTATTTATTGCTATAAGGCTCATCCAAAACGAAT | |
| | TAGCCGTTGCAaaagGTTGCAACACCTTGCAAAAATGGTGGTATATCCAAC | |
| 332 | ATTTAGCGTCCTGAGGCCGAGGGCTTTGACCTACTCGGCAAGGGTTAACCCTGGATGTTG | 43 |
| | TGTGACCGTCCAGGCGTTTCACATAGTCGGTTTCAAAACCAACTAAGTGAAATGTAAAT | |
| 333 | ATTTCATTGCCATGAAATTGGGCGATTTCACGTCCATAAGCCGAGAAAGTGGCCGCGTTT | 44 |
| | GATGCGGTTATACACTCGGCAACCCTGCACTATGACGAGTGCGAGGGATGAAAG | |
| 334 | ACATTTCACGCAAAATATAATTGCAGTAAAGCCAATTTaTATGGAATAAGCATTAAAGTA | 45 |
| | TTGAaTTTACAGTAaTTTGTAGTTATTaaagGTTATATATTAACTAGTGGAATGTAAAT | |
| 335 | GTTATATTTATTGTTGACTATTGGTTAGACGATGCGAAAGCAAAGGTTTAACACAAAACT | 46 |
| | CCTCCTATATCTATATGATATAGAAATGCGGCCGCTTGCTTCGGCCCTAAATTGAGCTGG | |
| | GAGGTCTCAAGACAAAGAATGCAAC | |
| 336 | ATTCAGCGACTAAAGGTTGAGGATATAGGATTAATAGCTAGGATAACCTAAAGCTATCTA | 47 |
| | TAATCACTCGATAAGGGTTAACTCTAGATGTTGTGTTACCGTCTAGACATACATATAGTTT | |
| | ATaaagATAAACTATGTGATATGTGAAT | |
| 337 | ATTATCTCAAGGTCCGGCTTaTTGTTGTATTTaAGCCGGCGTGTTCTTTGACATACGCGAAC | 48 |
| | ATTTATAGCAATGAAATGCGGGGTACGCGaaagGTCGCACCCCGCGCGTATGCGGGGGTTG | |
| | AAGG | |
| 338 | GGATTTACCAACAATACACTATTACTACTCACTAAGGGTAAGCCCAGGTGTTAAGTTACC | 49 |
| | GCCTGGCATACTAAGAATAGTAGTTTCAAAATTGCAGTAAATGATATTTGAAATAAAACA | |
| | TTGTAAACATAGTATATATAGGATGTTAGAGTATATTcTATAATaaagGTTATAAATCT | |
| 339 | GTTCTATAGGCGATTTAGCGTCTAAAGGTTGAGGGATAAGACAAAATGGTTAAGGTTTCG | 50 |
| | ACCAACTAACTACTTATCCACTCGATAAACGGTAAAAACTCATACTAATATTCTTTATAG | |
| | ATAACGTGGTGTGACATTCCAaaagTGGAATGTAAAT | |
| 340 | ATTACCACTCACTAAGGGTTAGCCTAGGTGTTATGTTACCGCCTAGCATTCTAAGATAGTT | 51 |
| | AAAAAATAAATTTGCAGTAGATGATCTCTTATAATTTAAGAGTTAAAACATAGTTaaagAAC | |
| | TATGTGGAATGTAAAT | |
| 341 | ATTACGATTAAATTCGTAGGGTAGATATTACTGCCTAAGGCTGAGGGTAAGGGAAAATGG | 52 |
| | TGTTGGTGTAAAACaaagGTTGTACAACACCCGCAATTGTAGTGGGTATAAAAC | |
| 342 | GTTCCAAAATTGAATAATTATATTAGTTTATTGTCTATCATAGATAGTGTAAAAAACACA | 53 |
| | GGGGGTAGACAAAATAAGTAATATAAGTATATGGTAGCTATTaaagAATAGCACCATAATG | |
| | GAATGTTAAT | |
| 343 | AATAAGTGAAAACTTACGGGCGATaTTGCGTCCGAAAAGTGAGGGTATTACCACTCACTA | 54 |
| | AAGTCTATGTAAATTTATCTATGTAATTTGAGGaTTTGGAGATATGTGATTaTACATAGAT | |
| | ACAAAAC | |

TABLE 6

| Target | Spacer Sequence | SEQ ID NO: |
|---|---|---|
| Kim-T1 | CACACACACAGTGGGCTACC | 423 |
| SMN2 B | CAAAAGTAAGATTCACTTTC | 424 |
| SMN2 A | AGGAGTAAGTCTGCCAGCAT | 425 |
| PRSS1 | TCGGCCAGGAACGGGGGGGT | 426 |
| PRSS1 | CAGTCGGGGGCCCCACACTT | 427 |
| DNMT1 | CTGATGGTCCATGTCTGTTA | 428 |
| FANCF1 | GGCGGGGTCCAGTTCCGGGA | 429 |
| TCRA | GAGTCTCTCAGCTGGTACAC | 430 |
| PDCD1 | GCACGAAGCTCTCCGATGTG | 431 |
| B2M | AGTGGGGGTGAATTCAGTGT | 432 |
| TCRA_mm1 | cAGTCTCTCAGCTGGTACAC | 433 |
| TCRA_mm2 | GtGTCTCTCAGCTGGTACAC | 434 |
| TCRA_mm3 | GAcTCTCTCAGCTGGTACAC | 435 |
| TCRA_mm4 | GAGaCTCTCAGCTGGTACAC | 436 |
| TCRA_mm5 | GAGTgTCTCAGCTGGTACAC | 437 |
| TCRA_mm6 | GAGTCaCTCAGCTGGTACAC | 438 |
| TCRA_mm7 | GAGTCTgTCAGCTGGTACAC | 439 |
| TCRA_mm8 | GAGTCTCaCAGCTGGTACAC | 440 |
| TCRA_mm9 | GAGTCTCTgAGCTGGTACAC | 441 |
| TCRA_mm10 | GAGTCTCTCtGCTGGTACAC | 442 |
| TCRA_mm11 | GAGTCTCTCAcCTGGTACAC | 443 |
| TCRA_mm12 | GAGTCTCTCAGgTGGTACAC | 444 |
| TCRA_mm13 | GAGTCTCTCAGCaGGTACAC | 445 |
| TCRA_mm14 | GAGTCTCTCAGCTcGTACAC | 446 |
| TCRA_mm15 | GAGTCTCTCAGCTGcTACAC | 447 |
| TCRA_mm16 | GAGTCTCTCAGCTGGaACAC | 448 |
| TCRA_mm17 | GAGTCTCTCAGCTGGTtCAC | 449 |
| TCRA_mm18 | GAGTCTCTCAGCTGGTAgAC | 450 |
| TCRA_mm19 | GAGTCTCTCAGCTGGTACtC | 451 |
| TCRA_mm20 | GAGTCTCTCAGCTGGTACAg | 452 |

TABLE 7

| SEQ | |
|---|---|
| ID NO: | Sequence |
| 453 | TTTCCCTTCAGCTAAAATAA |
| 454 | TCCGTGTTCCTTGACTCTGG |
| 455 | TTGGGTCAGCTGTTAACATC |
| 456 | TTCCCCACCTGGAGCAGGCT |
| 457 | CTCGCCTGTCAAGTGGCGTG |
| 458 | TGAACCTGGGTGAAGTCCCA |
| 459 | TTAATGTTAATAACTTGCTT |
| 460 | ACATTAACAAGAAGCATTTG |
| 461 | GAACATCCGCGAAATGATAC |
| 462 | CCAGGGCGAAGTGGGGAGGT |
| 463 | CCCTGGCTACCTCCCCTACC |
| 464 | CACACACACAGTGGGCTACC |
| 465 | GCTCAGCAGGCACCTGCCTC |
| 466 | GGGATTCCTGGTGCCAGAAA |
| 467 | TGCAGCCGCCGCTCCAGAGC |
| 468 | ACAGGCGAGTAACAGACATG |
| 469 | ACGTACTGATGTTAACAGCT |

TABLE 8

| sgRNA from | |
|---|---|
| FIG. 5 | SEQ ID NO: |
| A | 309 |
| B | 352 |
| C | 358 |
| D | 361 |
| E | 362 |
| F | 346 |
| G | 363 |
| H | 364 |
| I | 380 |
| J | 392 |
| K | 395 |
| L | 406 |
| M | 409 |
| N | 410 |
| O | 413 |
| P | 417 |
| Q | 419 |
| R | 313 |
| S | 351 |
| T | 368 |
| U | 369 |
| V | 353 |
| W | 384 |
| X | 404 |
| Y | 405 |
| Z | 407 |
| AA | 408 |
| BB | 411 |
| CC | 412 |
| DD | 414 |
| EE | 415 |
| FF | 416 |
| GG | 418 |

TABLE 9

| | | Nuclease tested | | | |
|---|---|---|---|---|---|
| tracrRNA | | SEQ ID NO: 21 | | SEQ ID NO: 20 | |
| label | modifications | R1 | R2 | R1 | R2 |
| 310 | WT | 0.8 | 0.6 | 1.8 | 1.9 |
| 366 | Δ12 | 1.5 | 1.2 | NA | NA |
| 365 | Δ14 | 1.6 | 1.3 | NA | NA |
| 364 | Δ16 | 1.1 | 1.3 | 5.8 | 8.3 |
| 363 | Δ18 | 2.8 | 2.4 | 6.7 | 6.2 |
| 346 | Δ20 | 4.5 | 6.3 | 25.1 | 24.9 |
| 361 | Δ22 | 3.1 | 3.7 | 10.9 | 14.2 |
| 362 | Δ24 | 1.4 | 1.7 | 4.3 | 4.6 |

TABLE 10
PAM sequence preferences

| Nuclease | PAM | Value |
|---|---|---|
| SEQ ID | ATTA | −2.55 |
| NO: 20 | GTTA | −3.09 |
| | ATTG | −3.2 |
| | GTTG | −3.31 |
| | TTTA | −3.56 |
| | TTTG | −3.69 |
| | CTTA | −4.39 |
| | CTTG | −4.51 |
| SEQ ID | TTTG | −3.56 |
| NO: 26 | CTTG | −3.82 |
| | ATTG | −3.95 |
| | TTTA | −4.28 |
| | ATTA | −4.35 |
| | CTTA | −4.77 |
| | GTTG | −5.53 |
| | GTTA | −6.75 |

TABLE 11
Constructs made for AAV study with nuclease of SEQ ID NO: 20 with sgRNAs targeting
PRSS1, SMN2, PCSK9 and TTR.

| Nuclease | Species | Gene | Guide | PAM | Sequence | SEQ ID NO |
|---|---|---|---|---|---|---|
| SEQ ID | human | PRSS1 | GSp342 | ATTA | AGAACACCATAGCTGCCAAT | 483 |
| NO: 20 | human | SMN2 | GSp251 | ATTA | AGGAGTAAGTCTGCCAGCAT | 484 |
| | mouse | PCSK9 | GSp376 | ATTA | TGAAGAGCTGATGCTCGCCC | 485 |
| | mouse | PCSK9 | GSp377 | ATTG | TGGTGCTGATGGAGGAGACC | 486 |
| | mouse | PCSK9 | GSp380 | ATTA | GGATTTGGGGTTTTGTCCTC | 487 |
| | mouse | TTR | GSp356 | TTTA | CAGCCACGTCTACAGCAGGG | 488 |
| | mouse | TTR | GSp368 | GTTG | CTGACGACAGCCGTGGTGCT | 489 |

TABLE 12
Amplification primer sequences

| | | SEQ |
|---|---|---|
| ID | Primer sequence 5′-3′ | ID NO |
| 4065 | ACACTCTTTCCCTACACGACGCTCTTCCGATCTGTCTGCAATGGACAGCTCCAAG | 490 |
| 4066 | GACTGGAGTTCAGACGTGTGCTCTTCCGATCTCCAGTGTGAAGGAGTGAGAGGG | 491 |
| 3974 | ACACTCTTTCCCTACACGACGCTCTTCCGATCTtAACTTCCTTTATTTTCCTTACAGGGT | 492 |
| 3975 | GACTGGAGTTCAGACGTGTGCTCTTCCGATCTCTTTAATATTGATTGTTTTACATTAAC | 493 |
| | CTTTCAAC | |
| 4071 | ACACTCTTTCCCTACACGACGCTCTTCCGATCTGAGTCCCAGGCGTCCATGTCCTTC | 494 |
| 4072 | GACTGGAGTTCAGACGTGTGCTCTTCCGATCTCATACCTTGGAGCAACGGCGGAAG | 495 |
| 3088 | ACACTCTTTCCCTACACGACGCTCTTCCGATCTGTGCATGCAACAAGACAACA | 496 |
| 3089 | GACTGGAGTTCAGACGTGTGCTCTTCCGATCTGAAGCCAGGGAAGAGGTCAT | 497 |
| 4073 | ACACTCTTTCCCTACACGACGCTCTTCCGATCTATGCTAAAGAGGTGGGGCTCAG | 498 |
| 4074 | GACTGGAGTTCAGACGTGTGCTCTTCCGATCTCCCCATCGGAAGATCCTCTCTG | 499 |
| 3956 | ACACTCTTTCCCTACACGACGCTCTTCCGATCTGTTCCAGAGTCTATCACCG | 500 |
| 3957 | GACTGGAGTTCAGACGTGTGCTCTTCCGATCTGCTTACCCAGAGGCAAAG | 501 |
| 4077 | ACACTCTTTCCCTACACGACGCTCTTCCGATCTTTGTCTGCAGCTCCTACCTCTG | 502 |
| 4078 | GACTGGAGTTCAGACGTGTGCTCTTCCGATCTTCTTCCTGAGCTGCTAACACGG | 503 |

The scope of the present invention is not limited by what has been specifically shown and described hereinabove. Those skilled in the art will recognize that there are suitable alternatives to the depicted examples of materials, configurations, constructions, and dimensions. Variations, modifications, and other implementations of what is described herein will occur to those of ordinary skill in the art without departing from the spirit and scope of the invention.

Numerous references, including patents and various publications, are cited and discussed in the description of this invention. The citation and discussion of such references is provided merely to clarify the description of the present invention and is not an admission that any reference is prior art to the invention described herein. All references cited and discussed in this specification are incorporated herein by reference in their entirety.

What is claimed is:

1. A composition comprising a nuclease, wherein the nuclease comprises a sequence with at least 70%, 80%, 85%, 90%, 95%, 96%, 97%, 98%, 99% or with at least 99% identity to any one of SEQ ID NOs: 1-250.

2. The composition of claim 1, wherein the amino acid sequence of the nuclease comprises any one of SEQ ID NOs: 1-250.

3. The composition of claim 1 or 2, wherein the nuclease further comprises a nuclear localization sequence (NLS) at the N-terminus, C-terminus, or both the N-terminus and C-terminus of the nuclease.

4. The composition of claim 3, wherein the NLS at the N-terminus and the NLS at the C-terminus of the nuclease are different sequences.

5. A nucleic acid comprising a first polynucleotide sequence encoding the nuclease of any of claims 1-4.

6. A vector comprising the nucleic acid of claim 5.

7. The vector of claim 6, further comprising a promoter operatively linked to the first polynucleotide.

8. The vector of claim 6 or 7, further comprising a second polynucleotide sequence encoding a guide RNA (gRNA).

9. The vector of claim 8, further comprising a promoter operatively linked to the second polynucleotide sequence.

10. The vector of claim 8 or 9, wherein the gRNA comprises a sequence with at least 60%, 70%, 80%, 85%, 90%, 95%, 96%, 97%, 98%, 99% or 100% identity to any one of SEQ ID NOs: 251-422 and 472-482.

11. The vector of any of claims 8-10, wherein the gRNA comprises any one of SEQ ID NOs: 251-343.

12. The vector of any of claims 8-10, wherein the gRNA comprises any one of SEQ ID NOs: 344-422.

13. The vector of any of claims 8-10, wherein the gRNA comprises any one of SEQ ID NOs: 472-482.

14. The vector of any one of claims 8-13, wherein the gRNA comprises a tracr sequence and the gRNA comprises one or more sequence deletions in or near the region encompassing the tracr sequence.

15. The vector of claim 14, wherein the one or more sequence deletions comprises sequences predicted to form a stem-loop structure.

16. The vector of claim 14 or 15, wherein the one or more sequence deletions comprises sequences predicted to form a stem-loop structure at or near 5′ end of the gRNA.

17. The vector of any of claims 14-16, wherein the gRNA comprises SEQ ID NO: 346.

18. The vector of any of claims 14-16, wherein the gRNA comprises SEQ ID NO: 420.

19. The vector of any of claims 14-16, wherein the gRNA comprises SEQ ID NO: 481.

20. The vector of any of claims 14-16, wherein the gRNA comprises SEQ ID NO: 479.

21. The vector of any of claims 8-20, wherein the gRNA comprises a spacer sequence of at least 18 nucleotides in length or between 18 and 20 nucleotides in length.

22. A system for modifying a target nucleic acid comprising:
a) a nuclease comprising an amino acid sequence having at least 70%, 80%, 85%, 90%, 95%, 96%, 97%, 98%, 99% identity or 100% identity to any of SEQ ID NOs: 1-250 or a nucleic acid encoding the nuclease; and
b) at least one guide RNA (gRNA) comprising a sequence complementary to at least a portion of a target nucleic acid and a region that associates with the nuclease, or a nucleic acid encoding the at least one gRNA.

23. The system of claim 22, wherein the nuclease is capable of recognizing a protospacer adjacent motif (PAM) sequence selected from the group comprising ATTA, GTTA, ATTG, GTTG, TTTA, TTTG, CTTA, and CTTG.

24. The system of claim 22 or 23, wherein the gRNA comprises a spacer sequence complementary to a first strand sequence of the target nucleic acid, and wherein the first strand sequence is directly adjacent to a protospacer adjacent motif (PAM) sequence selected from the group comprising ATTA, GTTA, ATTG, GTTG, TTTA, TTTG, CTTA, and CTTG.

25. The system of claim 23 or 24, wherein the PAM sequence comprises DTTR, wherein D is A, G, or T and R is A or G.

26. The system of any one of claims 22-25, wherein the nuclease is capable of preferentially modifying a target nucleic acid comprising PAM sequence ATTA as compared to a target nucleic acid comprising PAM sequence TTTR, wherein R is A or G.

27. The system of any one of claims 22-25, wherein the nuclease is capable of a higher efficiency of modification of the target nucleic acid as compared to the efficiency of modification of the target nucleic acid by nuclease SEQ ID NO: 471, wherein the target nucleic acid comprises PAM sequence is ATTA.

28. The system of any of claims 22-27, wherein modifying comprises nucleic acid cleavage.

29. The system of any of claims 22-28, wherein modifying comprises one or more of modification of the target nucleic acid, modulation of transcription from the target nucleic acid, and modification of a polypeptide associated with a target nucleic acid.

30. The system of any of claims 22-29, wherein the nuclease further comprises a nuclear localization sequence (NLS) at the N-terminus, C-terminus, or both the N-terminus and C-terminus of the nuclease.

31. The system of claim 30, wherein the NLS at the N-terminus and the NLS at the C-terminus of the nuclease are different sequences.

32. The system of any of claims 22-31, wherein the nuclease further comprises a purification tag.

33. The system of any of claims 22-32, wherein the at least one gRNA further comprises a sequence complementary to at least a portion of a second target nucleic acid.

34. The system of any of claims 22-33, wherein the at least one gRNA comprises a sequence with at least 60%, 70%, 80%, 85%, 90%, 95%, 96%, 97%, 98%, 99% identity or 100% identity to any one of SEQ ID NOs: 251-422.

35. The system of claim 34, wherein the at least one gRNA comprises any one of SEQ ID NOs: 251-343.

36. The system of claim 34, wherein the at least one gRNA comprises any one of SEQ ID NOs: 344-422.

37. The system of claim 34, wherein the at least one gRNA comprises any one of SEQ ID NOs: 472-482.

38. The system of claim 34, wherein the at least one gRNA comprises SEQ ID NO: 346.

39. The system of claim 34, wherein the at least one gRNA comprises SEQ ID NO: 420.

40. The system of claim 34, wherein the at least one gRNA comprises SEQ ID NO: 481.

41. The system of claim 34, wherein the at least one gRNA comprises SEQ ID NO: 479.

42. The system of any of claims 22-41, wherein the at least one gRNA comprises a spacer sequence of at least 18 nucleotides in length or between 18 and 20 nucleotides in length.

43. The system of any of claims 22-42, wherein the nuclease comprises SEQ ID NO: 20, and the at least one gRNA comprises a sequence with at least 60%, 70%, 80%, 85%, 90%, 95%, 96%, 97%, 98%, 99% identity or 100% identity to any one of SEQ ID NOs: 309, 346, 352, 358, 362-364, 380, 392-395, 410-420, 472-479, and 481, or any one of SEQ ID NOs: 352, 358, 363, 364, 380, 392, and 417, or any one of SEQ ID NOs: 346 and 362, or any one of SEQ ID NOs: 410-419.

44. The system of any of claims 22-43, wherein the nuclease comprises a sequence having at least 70%, 80%, 85%, 90%, 95%, 96%, 97%, 98%, or 99% identity to SEQ ID NO: 20, and wherein the at least one gRNA comprises any one of SEQ ID NOs: 309, 346, 352, 358, 362-364, 380, 392-395, 410-420, 472-479, and 481 or any one of SEQ ID NOs: 352, 358, 363, 364, 380, 392, and 417, or any one of SEQ ID NOs: 346 and 362, or any one of SEQ ID NOs: 410-419.

45. The system of any of claims 22-42, wherein the nuclease comprises SEQ ID NO: 21, and the at least one gRNA comprises a sequence with at least 60%, 70%, 80%, 85%, 90%, 95%, 96%, 97%, 98%, 99% identity or 100% identity to any one of SEQ ID NOs: 310, 344-349, 361-366, 404-422, and 479-482.

46. The system of any of claims 22-42, wherein the nuclease comprises a sequence having at least 70%, 80%, 85%, 90%, 95%, 96%, 97%, 98%, or 99% identity to SEQ ID NO: 21, and wherein the at least one gRNA comprises any one of SEQ ID NOs: 310, 344-349, 361-366, 404-422, and 479-482.

47. The system of any of claims 22-42, wherein the nuclease comprises SEQ ID NO: 22, and the at least one gRNA comprises a sequence with at least 60%, 70%, 80%, 85%, 90%, 95%, 96%, 97%, 98%, 99% identity or 100% identity to any one of SEQ ID NOs: 311, 346, 381, and 398-399.

48. The system of any of claims 22-42, wherein the nuclease comprises a sequence having at least 70%, 80%, 85%, 90%, 95%, 96%, 97%, 98%, or 99% identity to SEQ ID NO: 22, and wherein the at least one gRNA comprises any one of SEQ ID NOs: 311, 346, 381, and 398-399.

49. The system of any of claims 22-42, wherein the nuclease comprises SEQ ID NO: 23, and the at least one gRNA comprises a sequence with at least 60%, 70%, 80%, 85%, 90%, 95%, 96%, 97%, 98%, 99% identity or 100% identity to any one of SEQ ID NOs: 312, 346, and 382.

50. The system of any of claims 22-42, wherein the nuclease comprises a sequence having at least 70%, 80%, 85%, 90%, 95%, 96%, 97%, 98%, or 99% identity to SEQ ID NO: 23, and wherein the at least one gRNA comprises any one of SEQ ID NOs: 312, 346, and 382.

51. The system of any of claims 22-42, wherein the nuclease comprises SEQ ID NO: 24, and the at least one gRNA comprises a sequence with at least 60%, 70%, 80%, 85%, 90%, 95%, 96%, 97%, 98%, 99% identity or 100% identity to any one of SEQ ID NOs: 310, 313, 325, 346, 350-355, 358, 361-363, 367-372, and 389-392, or any one of SEQ ID NOs: 346, 352, 358, 361, 362, 368, 369, and 392.

52. The system of any of claims 22-42, wherein the nuclease comprises a sequence having at least 70%, 80%, 85%, 90%, 95%, 96%, 97%, 98%, or 99% identity to SEQ ID NO: 24, and wherein the at least one gRNA comprises any one of SEQ ID NOs: 310, 313, 325, 346, 350-355, 358, 361-363, 367-372, and 389-392, or any one of SEQ ID NOs: 346, 352, 358, 361, 362, 368, 369, and 392.

53. The system of any of claims 22-42, wherein the nuclease comprises SEQ ID NO: 25, and the at least one gRNA comprises a sequence with at least 60%, 70%, 80%, 85%, 90%, 95%, 96%, 97%, 98%, 99% identity or 100% identity to any one of SEQ ID NOs: 314, 346, 383, and 400.

54. The system of any of claims 22-42, wherein the nuclease comprises a sequence having at least 70%, 80%, 85%, 90%, 95%, 96%, 97%, 98%, or 99% identity to SEQ ID NO: 25, and wherein the at least one gRNA comprises any one of SEQ ID NOs: 314, 346, 383, and 400.

55. The system of any of claims 22-42, wherein the nuclease comprises SEQ ID NO: 26, and the at least one gRNA comprises a sequence with at least 60%, 70%, 80%, 85%, 90%, 95%, 96%, 97%, 98%, 99% identity or 100% identity to any one of SEQ ID NOs: 315, 346, 384, 392, 396-397, 420, 479, and 481, or any one of SEQ ID NOs: 346, 384 and 392.

56. The system of any of claims 22-42, wherein the nuclease comprises a sequence having at least 70%, 80%, 85%, 90%, 95%, 96%, 97%, 98%, or 99% identity to SEQ ID NO: 26, and wherein the at least one gRNA comprises any one of SEQ ID NOs: 315, 346, 384, 392, 396-397, 420, 479, and 481, or any one of SEQ ID NOs: 346, 384 and 392.

57. The system of any of claims 22-42, wherein the nuclease comprises SEQ ID NO: 27, and the at least one gRNA comprises a sequence with at least 60%, 70%, 80%, 85%, 90%, 95%, 96%, 97%, 98%, 99% identity or 100% identity to any one of SEQ ID NOs: 316, 346, 385, and 401.

58. The system of any of claims 22-42, wherein the nuclease comprises a sequence having at least 70%, 80%, 85%, 90%, 95%, 96%, 97%, 98%, or 99% identity to SEQ ID NO: 27, and wherein the at least one gRNA comprises any one of SEQ ID NOs: 316, 346, 385, and 401.

59. The system of any of claims 22-42, wherein the nuclease comprises SEQ ID NO: 28, and the at least one gRNA comprises a sequence with at least 60%, 70%, 80%, 85%, 90%, 95%, 96%, 97%, 98%, 99% identity or 100% identity to any one of SEQ ID NOs: 317, 346, 386, and 402.

60. The system of any of claims 22-42, wherein the nuclease comprises a sequence having at least 70%, 80%, 85%, 90%, 95%, 96%, 97%, 98%, or 99% identity to SEQ ID NO: 28, and wherein the at least one gRNA comprises any one of SEQ ID NOs: 317, 346, 386, and 402.

61. The system of any of claims 22-42, wherein the nuclease comprises SEQ ID NO: 29, and the at least one gRNA comprises a sequence with at least 60%, 70%, 80%, 85%, 90%, 95%, 96%, 97%, 98%, 99% identity or 100% identity to any one of SEQ ID NOs: 318, 346, 387, and 403.

62. The system of any of claims 22-42, wherein the nuclease comprises a sequence having at least 70%, 80%, 85%, 90%, 95%, 96%, 97%, 98%, or 99% identity to SEQ ID NO: 29, and wherein the at least one gRNA comprises any one of SEQ ID NOs: 318, 346, 387, and 403.

63. The system of any of claims 22-42, wherein the nuclease comprises SEQ ID NO: 36, and the at least one gRNA comprises a sequence with at least 60%, 70%, 80%, 85%, 90%, 95%, 96%, 97%, 98%, 99% identity or 100% identity to any one of SEQ ID NOs: 310, 313, 325, 346, 356-360, and 373-378.

64. The system of any of claims 22-42, wherein the nuclease comprises a sequence having at least 70%, 80%, 85%, 90%, 95%, 96%, 97%, 98%, or 99% identity to SEQ ID NO: 36, and wherein the at least one gRNA comprises any one of SEQ ID NOs: 310, 313, 325, 346, 356-360, and 373-378.

65. The system of any of claims 22-64, wherein the nucleic acid molecule encoding each one or both of the nuclease and the at least one gRNA comprises a messenger RNA, a vector, or a combination thereof.

66. The system of any of claims 22-65, wherein the nuclease and the at least one gRNA are encoded on one nucleic acid.

67. The system of claim 66, wherein the nuclease and the at least one gRNA are operatively linked to different promoters.

68. The system of claim 66 or 67, wherein the one nucleic acid is a vector.

69. The system of claim 68, wherein the vector is a viral vector.

70. The system of claim 69, wherein the viral vector is an AAV vector.

71. A kit comprising the system of any one of claims 22-70.

72. A cell comprising the system of any one of claims 22-70.

73. The cell of claim 72, wherein the cell is a prokaryotic or eukaryotic cell.

74. The cell of claim 72 or 73, wherein the cell is a mammalian cell.

75. The cell of any of claims 72-74, wherein the cell is a human cell.

76. A method of modifying a selected target nucleic acid sequence comprising contacting the selected target nucleic acid with a composition of any one of claims 1-4, a nucleic acid of claim 5, a vector of any one of claims 6-21, or a system of any one of claims 22-70.

77. The method of claim 76, wherein the target nucleic acid sequence is in a cell.

78. The method of claim 77, wherein the cell is a prokaryotic or eukaryotic cell.

79. The method of claim 77 or 78, wherein the cell is a mammalian cell.

80. The method of any of claims 76-78, wherein the cell is a human cell.

81. The method of any of claims 76-80, wherein the contacting comprises introducing the composition of any one of claims 1-4, the nucleic acid of claim 5, the vector of any one of claims 6-21, or the system of any one of claims 22-69 into the cell.

82. The method of any of claims 75-80, wherein the contacting comprises administering introducing the composition of any one of claims 1-4, the nucleic acid of claim 5, the vector of any one of claims 6-21, or the system of any one of claims 22-70 to a subject.

83. The method of any of claims 76-82, wherein the selected target nucleic acid sequence encodes a gene product.

84. A composition of any one of claims 1-4, a nucleic acid of claim 5, a vector of any one of claims 6-21, or a system of any one of claims 22-70 for use in modifying a selected target nucleic acid sequence.

85. A kit comprising composition of any one of claims 1-4, a nucleic acid of claim 5, a vector of any one of claims 6-21, or a system of any one of claims 22-70 for use in modifying a selected target nucleic acid sequence in an in vitro assay.


SEQUENCE LISTING (510 sequences)

SEQ ID NO: 1 - protein, 429 aa, unidentified
MTKVIKLALICQQSDSNGMPVDYKEVNKILWELQRQTREIKNKSIQYCWEYHNFSSDYYKRNGEYPKEKDVLLFTLGGYVNDKFKTGNDLYSANCSTTVRGVCGEFKNSKKDFISGKRSIISYKENQPLDLHNKSIRLEYSDHEFYVYLKLLNRQGFKKFNFADTQIMFKILVRDNSTKTILERCLDEVYSVSASKLIYDKKKKCWVLNLSYSFSGEITHNLDENRILGVDLGIHYPICASVYGEWKRFTIDGGEIEEYRRRVEARKKTLLKQGKNCGDGRIGHGVKTRNKPVYSIEDRISRFRDTANHKYSRALINYAIKNNCGVIQMENLEGVTAHSDKFLKNWSYYDLQTKIEYKAKEAGIKVVYINPRYTSQRCSKCGYTDTDNRPEQAKFICKKCGFSENADFNASQNIGIKNIEQIIKEEIQI

SEQ ID NO: 2 - protein, 438 aa, unidentified
MQKVLKVYLICEQLDPDGSPVDYKDIFKLLWDLQKQTREIKNKSIQYCWEFSNFSSDYYKEHHEYPKDKEILNYTLGGFVNDKFKTGNDLYSANCSTTVRTACAEFKNAKADFMRGEKSIISYKANQPLDLHNKSIRLEYQKGTFLFYLKLLNRSAVKKHGFQSSEIRFKAIVKDNSSQTILERCTAGVYDMAASKLLYDQKKKCWVLNLVYAFEPETPEALDPEKILGVDLGVHYPICASVYGDLKRFIIDGGEIESFRKRVEARKISMLKQGKNCGGGRIGHGIQTRNKPVYAIADKIARFRDTVNHKYSRALIDYAIKNNCGVIQMEKLTGVTADANRFMKNWTYFDLQTKIEYKAQEAGIQIVYIEPKYTSQRCSKCGYIDRENRPEQSKFICRKCGFSENADYNASQNIGIRNIEKLIEKQLQTKCESETDTT

SEQ ID NO: 3 - protein, 424 aa, unidentified
MKKTVRLQIVKPMDEDWEILGRVLHDIRYQTRQVLNKTIQLCWEYSNFSSEYKALHGDYPKNKEILHYTSMHGYAYNQLKEQYYYIQSGNCSQTVKRAVDKWKSDLKEILRGDRSIPSYKKDIPIDIVKDAVSLEHDEKNGNYIATLSLMSTAYRKEMERKSGQFRVLLHSGDNSKKTILKRLVQGEYQHTASQIVKKGKKWFLNLSYKFDVLQTPFQTERVMGVDMGVIYPIYMAFNYHDHLRYKIQGGEIERFRRQVESRKKALQDQGKYAGSGRVGHGTKTRIDPLEVIRDKIANFRETKNHHYSRYVVDMAEKHECATIQLEELKGIHQDDAFLKRWSYHDLQEKITYKAEEKGIQVIKVDPQKTSQRCHHCGNIDSNNRKEQASFLCTSCGMETNADFNAAKNISIPGIEQIIQTEMKS

SEQ ID NO: 4 - protein, 430 aa, unidentified
MCALTKIMKYELRYLDGFPDFSAMQNAVWPLQRQTREILNRTIQEAYRWDYFSATKKKETGEYPDLQKETGYKRLDGYIYHVLSPDYPDFSSSGVNATIQKAWKKYKSSKADVWKGEMSLPSYKSDQPIVLHAKQIKLSGDTRAAAATLSLFSNKFKKEHEISGNVQFAITLHDNTQRTIYQKLRNGEYKLSESQLVYDKKKWFLYLAYSFNPAEHALDPEKILGVDMGEKFALYASSFGEYGHFKIEGSEVTEYAKALERRRRSLQQQARYCGEGRIGHGTKTRVGVVYREEDRIANFRSTINHRYSKALIEYAVKNGYGTIQMENLTGIKENLQFPRRLQHWTYYDLQSKIEAKAKEHGIAVVKVNPKHTSQRCSRCGHIAAENRPKQEVFQCVKCGYACNADFNASQNISIKDIEKLIQETIGANPK

SEQ ID NO: 5 - protein, 427 aa, unidentified
MVLTRKLQLVPCAEGMSKEEAKKEVDRVYKILRDGIYAQNKAYNIFISRRYTAILLGASKEELAKLTLIGERNPKKDDPSYSLYEYGKINFIKGIPQASALGQHAISDLSKQKKDGLFKGKVALACRRLDAPMWIKQKYEFYHNYADNKELSENLYSEDLKIYMKLANICVFEVVLGNPHKSAAIRAELERVFDENYKKLDSSIQIVNNKIMLYLAINIPEKQIELKEDVVVGVDLGIAIPAVCALNNSRYIKKSIGSANEFLRIRTQLQSEKKRLQRKLRDTNGGHGRKKKLAPLDKLSKRERNFVQTYNHMISRRVVDFAVKNNAKYINVEDLSDYKNNGSEYILRNWSYYELQQQIEYKAEMYGIVVRKVNPYHTSQICSQCGHWEEGQRKSQSEFECKACGYTANADFNAARNIALSTDFVKK

SEQ ID NO: 6 - protein, 433 aa, unidentified
MSKGVVTKVMKYTLRYIGGCGDFHKMQEAVWKLQRQTREVLNKTVQLAFDWDHRSREAYRTSGEYLDIVKETGYKRLDGYIYNRLKTDYADFASSNLNATIQVAWKKYMASKSDILTGKMSFPSYKSNQPIVLHNSSIRFSTEKYGVPAAELTVFSNALKKENGLSTNPAFEILLMDGTQRSIFQRVISGEYKHGQCQINYEKRKWFLYLTYTFEAGKTPLDPDKILGVDIGETLAICASSTSEWGRFVIQGGEVTRYSKQIEERRRSQQRQATYCGEGRIGHGTKARVAPIYATEDRIANFRDTINHRYSKALIEYAVKHGFGTIQMEDLSGIKGEKDFPKFLRHWTYYDLQNKIEAKAKEQGINVVKVQPAYTSQRCSKCGCIDKENRKNQELFCCVKCGFKANADFNASQNISIKGIDKIIEKEYNANIE

SEQ ID NO: 7 - protein, 437 aa, unidentified
MNKVVKLALISKVKDKDGNDVKYGDVCKILWELQRQTREIKNKAVQFCWEWNGFSSEYNNLFGEYPKDKDYLKNKSEGKPIVLRSFVYDRLKSDYYLNSSNLSTTTSLAFKEFKQYLTDIRKGERSVLNYKNNQPLELHNECIWLESNNGKFITRFSFLNKAGKDFYSIDNFTFEVIVKDNSTKTILERCIDSIYGIRASKLIYNQKKKQWFLNLSYSFEAKEIATLDKDKILGVDLGIALPICASVYGDLDRFTIKGGEIEHFRKSIESRKRSLLQQGKVCGDGRIGHGIHTRNKPAYNIEDKIARFRDTANHKYSRALIDYAVRKGCGTIQMEMLKGITEEKDPFLKNWTYFDLQQKIEYKAKEKGIKVVYIAPKYTSRRCSKCGHIDKDNRLTQANFLCLNCGYKENADYNASQNIAIKDIDKIIIEETKGGES

SEQ ID NO: 8 - protein, 434 aa, unidentified
MNKVVKLSLICEQTDKDGNKIEHGEVYKILWELQRQTREIKNKTIQYCWEYSNFSSDYYKINHEYPNEKEILSFTLKGFVNDKFKKGNDLYSGNCSTTTGNVCSEFKNSKSDFLKGEKSIINYKAYQPLDIHNKCILIEHTNNEFYVRLKLLNRPAIKKYNFANSEFNFKIIVKDNSTRTILERCIDKIYDVAASKLIYDKKKKMWVLNLVYAFDNKSEYVLDKNRILGVDLGIHYPICASVYGEWNRFTIDGGEIEKFRKTVEARKKSMLRQGKNCGDGRIGHGISARNKPVYKIEDKIAKFRDTANHKYSHALIQYAIKNNCGVIQMEDLTGITNEADRFLKNWSYYDLQTKIKYKAKEVGIDIVYIKPKYTSQRCSKCGYIDKENRNKQASFVCLKCGFKANADYNASQNISIKDIDKLIEEMYNSSANTE

SEQ ID NO: 9 - protein, 432 aa, unidentified
MSKGSLAKVMKYELRYLDGAGSFEQMQERLWVLQRQTREILNRSTQISFHWDYTSREHFEQTGQYLDVFSETGYKRLDGYIYSRVKDSCGDMASGNINATLQKAWNKYGTSKLDVLRGQMSLPSYKKDQPLVIEKHNIRLSMDGQQALAEITLFSNKFKKENSLSSNVRFAFQLHDGTQRRILNSVLSGEYGLGQCQLVYDRPKWFLLLTYTFTPQNRQLDPDRILGVDLGECYALCASVFGEYGSLRIEGGEVTAYAKKLEARKRSLQKQAAVCGEGRKGHGTKTRVADAYQMQDRIANFRDTVNHRYSKALIDYALKNQCGTIQMEDLSGIRQDTGFPKFLQHWTYYDLQSKIENKAKEHDIRIVKINPRYTSQRCSKCGAIDSGSRTSQARFCCTKCGFTANADYNASQNISIKGIDLLIEKELGAKAE

SEQ ID NO: 10 - protein, 438 aa, unidentified
MGKGEISKVMKYELRYLDGSGSFEEMQQRVWALQRKTREIQNRTVQIAFHWDYINREHFIQTGNNLNVLQETGYKRLDGYIYDRLKGQSAEMSGANLNATIQTAWKKYNSAKPKVLSGTMSVPSFKRDQPLIINSNCVKFSRSESECLAELTLFSREYKKEHDLSSNVRFAIRLHDSTQRSILERVLSGAYRKGQCQLVYQRPKWFLFLTYSFFPMQHDLDPEKYLGVDLGECCALYASSVGEYGSLKLEGGEITAFAKQLEARKRSMQKQAAYCGEGRIGHGTKTRVADVYKMENRIANFRDTVNHRYSKALIDYAVKHQYGTIQMEDLSGIKNDTGFPKFLRHWTYFDLQEKIDAKAREHGIHVVKVNPQYTSQRCSKCGSIDSRNRKSQKEFCCLNCGYKVNADFNASQNLSIKGIDVIIQKYIGAKSKQTENNG

SEQ ID NO: 11 - protein, 430 aa, unidentified
MKEIAKVVKLELGWVFTDDGERAFPYSDLFEIQRQVALVKNKTIQLCWEWNNFGADFHAMSGAYPKTGDVTGYKTLDGYVYQRLKDDFSRMFKKNLNASVRSAQKAFETAKKSLHKGERSILSYRKDAPIELHNSAVSFSQEGRKYKAAVKVFSLSYAKEKGYAGTGVEFELSHLQGSPKEIVQRCMSGEYKIGESKLIWNEKKKKWFLYLTYKFTPAAVALDPEKIMGVDLGIACVAYMGFRFCEDRHVIPGHEVEHFRRRVEARKVELQRQGKFCGEGRIGHGRATRTKPVDQIGHAIARFRDTANHKYSRFIVDMAVKHGCGVIQMEDLHVHAEDKLLKDWTYFDLRTKLEYKAKEKGIEVRFVNPRYTSQRCSCCGYIEKENRKTQKEFICLECGFAANADYNAALNLATAGIEQIIDEYVSANHK

SEQ ID NO: 12 - protein, 432 aa, unidentified
MSKGTLAKVMKYELRYLDGFPSFYEMQEAVWGLQRQTREILNKTLQMAFHWDYTSREHFKETGSYLDVRTETGYKRFDGYVYECLKSDYSDIASKNLNATLQKAWKKYRNTRLDVLKGTMSLPSYRSDQPLTLDKNTVKLHSDGVDDWVELTLFSKAYKTQHGLSANVRFAIPMHDRTQRSIFQNLIDGVYALGECQLVYDKKKWFLLVTYVFTPEQHTLDPEKILGVDMGEAYAIYASSVYGYGTLKIEGGEVTDYAKKLERRKWSYQKQARYCGDGRIGHGTKTRIAEVYKAEDRIANYRDTINHRYSKAVVDYAVKNGYGTIQMEDLSGIKEDTGFPRRLQHWTYYDLQTKIENKAKEHGIRVVKIDPRCTSQRCSRCGHIDPKNRPSQSQFCCTACDFRANADFNASQNISTKGIDKIIAKTLRAKPE

SEQ ID NO: 13 - protein, 430 aa, unidentified
MGTVTKVMKYELRYLDGSGSFHDMQNYVWQLQRQTREILNRTIQEATLWDYRSREHFLEAEEYLDVYAETGYKTLDGYIYNRLKGSYGDFAGANLNATLRKAWKKYKTSKTEVLRGTMSLPSYKGDQPLVLHNGSVKLQGDSRDAVVELTLFSNTFKKREGIKGNPSFSLLVRDNTQRSIYQSLIDGVYKLGECQLVYQKKKWFLLLTYTFEARQHEVDPEKILGVDLGEAYAIYASSKDNFGSLKIEGGEVTDYAKGLERRKRALQQQARYCGEGRVGHGTKTRVTEAYKAEDRLANFRKTINHRYSKALIDYAVKNGYGTIQMEDLSGIKADTGFPKRLQHWTYYDLQSKIEAKAKEYGIHVVKVDPSYTSQRCSKCGHIDSQNRKTQERFLCVSCGFSCNADFNASQNLSIKGIEKIIKKTKGAKVE

SEQ ID NO: 14 - protein, 455 aa, unidentified
MAKKGNSQKKQIVKVMKYELKYEKGCADFNEMQNELWKLQRQTREVMNRTIQLCYHWSYVQAEYCKQHGCARRDVKPCDVYETNATSLDGYIYQLLKVEYPDFFMKNLNATLRKAHQKYDALLFDIQEGNSSIPSFKKDQPLIFEKKAICISKCLPDKRQITLSCFSDSYIDAHPTLDKITFTVRARSASEKSIFDHIISGKYALGTSQLVYEKKKWFFLLSYKFTPESVDVNPEKVLGVDLGVVNALCAGSVENPHDSLFIKGTEAIEQIRRLEARKRDLQKQARYPGDGRIGHGTKTRVSPVYQTRDAIARMQDTLNHRWSRALIDFACKKGYGTIQMEDLSGIKAMESEKPYLKHWTYFDLQSKIIYKAEEKGIRVVKVNPKCTSRRCSACGYISKENRKNQAEFLCVNCGYHHNADYNAAQNLSIPQIDRLIEKQLKEQESEESEAGTNPK

SEQ ID NO: 15 - protein, 441 aa, unidentified
MDLRNWLRRVRIHMAKGTVTKVMKYELRYLSGFSDFHAMQQAVWGLQRQSREILNKTIQMAFHWDYISRENFNANGVYLDVKAETGYKTYDGYIYNSLKSAYADMAAANLNAAIQKAWKKYKDAKMEVLRGTMSTPSYRSDQPVLINKNCVKLFDGGVRLTLFSDRFKRENNLNGNLEFAVQLHDGTQRSIFANLLNGTYALGQCQLVYDKRKWFLLVTYIFTPEKHELDPEKILGVDLGQTYALYASSVCARGTFRIEGGEAAECAHRLEQRKRSLQQQARFCGEGRVGHGTKTRVAAVYSAGDKIASYRDSINHRYSKALVEYAVKNGYGTIQMEDLTGIQNDLDHPKRLQHWTYYDLQTKIENKAKEHGVGVVKVNPRYTSQRCSRCGHIERENRPTQKVFCCKACGFEGNADYNASQNLSMRNIDKIIEKELSAKGE

SEQ ID NO: 16 - protein, 437 aa, unidentified
MSGSTIAKVMKFELFYREGGGEFHEMQKLLWELQRQTREVLNKSVQIYYQWKWKKQQHFEETGQSLDIYTELHYHRISSYAYNVLKEKYSSFYKANLSSTIKTACDKCESSEKDILCGTMSVPSYKRDQPLLLHDTSLSIRRNGTQWFADCKLFSAELVKNLGLKRGQSLVFSIKALDKTQINILERIEDGNYAIRQSQLTYEKKKWFLYLTYRFDKPKSELDPNRILGVDLGVSNAFCASVYGELDKLMIPGDEAIETIRRLEQIKYSKLRQARYCGEGRIGHGTSTRIAPAYSTRDKISNLQKTLNHRWSHAIVRYALRQGCGVIQMEDLSGIKEANDFPLRLQHWTYYDLQTKIKNKANEYGIEVRQVDPQFTSQRCSKCGCIQKENRPAQAKFCCIKCGYRTNADYNASQNLALPDIDRIIQEELKQIGANRK

SEQ ID NO: 17 - protein, 437 aa, unidentified
MQQVVRFEILKPVDNDWKILGRVFRELQYESRLVLNKTIQYNWEFSNCVIGFKEKFGIAPKISDISKYKDGKRGLEGYIYDKLKDIYTKNYSKNLGCLISKATSQWYAIKNDVYKGEKIPPEYKKSNTPIIVDKQAMTLFKENNLYYAKVALVSTNHIKEYGLNSCKFTILLNTKNNGNKVILDKVINGEFDYSQSQIIEKNKKWFLYLSYKIKEKTLPDDYNRVMGIDLGKNKAVVIAVHGTEIRDYILGGEIIDYKKKMYNMVWHRQKQSRYCGEGRIGHGRKTRLKDVYNIKEKIANFSDLTNHKYSKYIVELAKRHKCGIIQMEDLSGLSTDNKFLKQYPIYDLQQKIIYKAEREGIKVVKIKPHFTSQMCSNCHYISTQNRPKDDRGWEYFKCVNCGLEIDADLNAARNIANPQIELIIEEQLKIQEIDDKI

SEQ ID NO: 18 - protein, 437 aa, unidentified
MAEKTIVKVMKFELRYIDGAGEFSEMQKHLWELQKQTREVLNKTIQMGYALECKRFAHHDKTGQWLDDKELTGSKYKAVADYINAELKEDYNIFYSDCRNSTVRKAYKKFKDAKNKIFSGEMSLPSYRSNQPIIIHNRNVIIRGNAESALVGLKVFSDGFKALHGFPAAVNFKLCVKDGTQRAIIENVISEIYKISESQLIYDNKKWFLILAYRFTQKKNDLNPDKILGVDLGVKFAVYASSIGEYGSFRIKGGEVTEFIKRLEKRKKSLQNQATVCGDGRIGHGTKTRVADVYKARDKISNFQDTINHRYSRAIVDYARKNGYGTIQLEKLDNSIEKKGDYSPVLVHWTYYDLRTKMEYKAAEYGIKVIAVEPKYTSQRCSKCGYISSENRKTQESFECIKCGYKCNADFNASQNLSVRDIDRIIDEYLGANPELT

SEQ ID NO: 19 - protein, 488 aa, unidentified
MANEFTCITRKIEVHLHKHGDSDEAIQRYKEEYRMWDDINNNLYKAANRIVSHCFFNDTYEYRLKLHSPRFQEIEKLLSNPKRNKLSDDDIKELKAERKLLFSDFKSQRQTFLRGGIETGTNPEQNSTYKVISNEFIDCIPSEVLTNLNQNISSTYREYTLDVERGIRTIPNFKKGIPVPFSIKQHGEIALKKRDDGTIYVRFPKGLEWDLNFGRDRSNNREIVERVLSGQYGVGNSSIQESKNKKQFLLLVVKIPKENRVLDKERIVGVDLGVNTPLYAALNDNEYGGMGIGSREQFLKVRERMNAQKRELQRNLRHSTNGGHGRSQKLQALDRLEGKERNWVHLQNHIFSKSIIEYALKNDAGVIQMERLTGFGRDNNEEVQNEYKYILRYWSYFELQTMIEYKAKAAGIEVRYINPYHTSQTCSFCGHYEKGQRINQPTFICKNPDCTKGKGKQKSNGAYEGINADWNAARNIARSNEFVEKKKK

SEQ ID NO: 20 - protein, 486 aa, unidentified
MATEYTCITRKIEVHLHRHGDSEEDTQRLKDEYHIWDVINDNLYKAANRIVSHCFFNDAYEYRLKLHSPRFQEIEKLLRYSKRNKLTDEDIKQLKAERKELFSIFKKQRLEFLQGGSGKGSEQNSTYKVVSNEFGEIIPSHVLTCLNQNITSTYSAYSKEVEYGNRTIPNFKRGIPVPFPIKQQGTLQLKRREDGSIYIRFPLGLEWDLSFGRDRSNNREIVERVLNGQYDVGNSSIQETKNKKRFLLLIVKIPKQAVTLNPDRIVGVDLGINIPLYAALNDNEYGGMGIGSREQFLKMRMRMAAQKRELQRNLRHTTHGGHGRTQKLQALERLEGKERNWVHLQNHIFSKSIIEYAQRNDAGVIQMERLTGFGRDKHDEIDSDFKFILRYWSFFELQTMIEYKAKAAGIEVRYIDPYHTSQTCSFCGHYEKGQRISQSTFVCKNPDCEKGKGKKHSDGTYEGINADWNAARNIALSTKIVDRKKK

SEQ ID NO: 21 - protein, 488 aa, unidentified
MATEYTCITRKIEVPLHRHGEDEEAKQRLIDDYRVWDTINDNLYKAANRIVSHCFFNDAYEYRLKIHSLRFQEIEKLLKYSKRNKLTDEDIKQLKAERKQLFADFKKQRHTFLRGGVAEGANPEQNSTYKVISNEFLEVIPSEILTNLNQNISSTYKNYSLDVERGIRTIPNYKRGIPVPFSIKQRGELMLKRRDDGSIFIRFPMGLEWDLSFGRDRSNNREIVERVLSGQYDVGNSSIQESKNRKRFLLLVVKIPKENHNLNPDRIVGVDLGINIPLYAALNDNEYGGMGIGSREQFLNMRMRMDAKKRELQRNLRQSTNGGHGRKQKLQALERLEGKERNWVHLQNHIFSKSIIEYAVKNNAGAIQMERLTGFGRDKNDEVDSDFKFILRYWSFFELQTMIEYKANAAGIEVRYIDPYHTSQTCSFCGHYEKGQRLNQSTFVCKNPDCEKGKGKKLSNGTYQGINADWNAARNIALSDKIVDRKKK

SEQ ID NO: 22 - protein, 488 aa, unidentified
MATEYTCITRKIEVHLHKHGDSEEAAQRFKEEYRIWDDINNNLYKAANRIISHCFFNDTYEYRLKLHSPRLQEIEKLLSNPKRNKLSDEEVKQLKAERKQLFADFKKQRHVFLRGGVEEGANPEQNSTYKVVSNEFIDFIPSEVLTNLNQNISSTYREYSLDVERGVRTIPNYKKGIPVPFSIKQKGEIVLKKREDGSMYVRFPKGLEWDLNFGRDRSNNREIVERVLSGQYDVGNSSIQETKNKKRFLLLVVKIPKQVASFDPSRIVGVDLGINVPLYVAINDNEYGGMGIGSREQFLKMRMRMAAQKRELQRNLRHTTNGGHGRTQKLQALNRLEGKERNWVHLQNHIFSKSIIEYAVRNNAGVIQMERLTGFGRDKNDEVGADFKFLLRYWSFFELQSMIEYKAKATGIEVRYINPYHTSQTCSFCGHYEKGQRINQATFVCKNPECTKGKGKQRTDGTFEGINADWNAARNISFSTDFVDKKKK

SEQ ID NO: 23 - protein, 481 aa, unidentified
MATEYTCITRKIEVHLHRHGDSEEAAQRLKEEYRIWDEINDNLYKAANRIISHCFFNDAYEYRLKLHSPRFKEIERLLKYAKRNKLTDDDIKALKAERKELFAEFKRQRQSFLGGSEQNSTDRVVSHEFLDVIPSEVLTCLNQNIASTYKEYARDVERGVRTISNFKKGIPVPVRVKRNGALLLRKREDGSIYLSFPKGLEWDLNFGRDRSNNREIVERVLSGQYDVGGSSIQEAKNGKRFLLLVVKIPKESRALNPDRVVGVDLGVNIPLYAALNDNTYGGLSIGSRDQFLKVRMRMAAQKRELQRNLRVATNGGHGRKQKLQALDRLEGKERNWVHLQNHIFSKSIIEYALRNEAGAIQMERLTGFGHDRNDEVDEGFKFILRYWSFFELQTMIEYKAKAAGIEVRYVDPYHTSQTCSFCGHYEKGQRVNQATFICKNPDCTKGKGKERSDGTFEGINADWNAARNIALSDKIVERKKK

SEQ ID NO: 24 - protein, 488 aa, unidentified
MATEYTCITRKIEVHLHKHGDSEEATQRLKNEYHIWDEINNNLYKAANRIVSHCFFNDTYEYRLKLHSPRFQEIEKLLNNSKRNNLSAEEIRQIKIERKLLLSEFKKQRYAFLRGGIEEGANPEQNSTYKVVSNEYIDKIPSDVLTNLNQNISSTYKEFSLDVEKGVRTIPNYKKGLPIPFSIKKNGDLLLKKRDDGTIYIRFPKGLEWDLSFGRDRSNNREIVERILSGQYDVGNSTIQETGNKKRFLLLVVKVPKKNIVSNPNRVVGVDLGINYPLYAALNDNEHGGISIGSRDQFLKMRMRMAAQKRELQRNLRHTTNGGHGRTQKLQALERLEGKERNWVHLQNHIFSKSIIEYAIKNNAGTIQMERLTGFGRNENNEVGSEYKFLLRYWSFFELQTMIEYKAKASGIDVRYINPYHTSQTCSFCGHYEKGQRLNQATFVCKNSACTKGKGKQKSDGTYEGINADWNAARNIALSTDFVDKKKK

SEQ ID NO: 25 - protein, 481 aa, unidentified
MATEYTCITRKIEVHLHRHGDSEEAAQRLKEEFRIWDEINDNLYKAANRIISHCFFNDAYEYRLKLHSPRFQEIEKLLKYAKRNKLTDDDIKALKAERKELFAEFKRQRQSFLGGSEQNSTYKVVTDEFLEVIPSHVLTCLNQNISSTYREYALDVEHGRRTIPNFKKGIPVPFPIKATGELLLRKREDGSIYIRFPKGLEWDLNFGRDRSNNREIVERVLSGQYDVGNSSIQETKNRKRFLLLVVKIPKESRALNPDRVVGVDLGVNIPLYAALNDNTYGGMSIGSRDQFLKVRMRMAAQKRELQRNLRVATNGGHGRKQKLQALDRLEGKERRWVHLQNHIFSKSIIEYALRNEAGAIQMERLTGFGHDRNDEVDEGFKFILRYWSFFELQTMIEYKAKAAGIEVRYVDPYHTSQTCSFCGHYEKGQRVNQATFICKNPDCTKGKGKERSDGTFEGINADWNAARNIALSDKIVERKKK

SEQ ID NO: 26 - protein, 488 aa, unidentified
MATDVTCITRKIEVHLHKHGDSEEGAQRLKEEYRIWDDINNNLYKAANRIISHCFFNDTYEYRLKLHSPRFQEIEKLLSKPKCNKLSADDIKQLKAERKVLFADFKKQRQVFLRGGLEEGTNREQTSTYRVASKEFIDTIPSEVLTNLNQSISSTYKKYALEVERGVRTIPNYKKGIPVPFAIKHKEELALKKRDDGSIYVRFPKGLEWDLSFGRDRSNNREIVERVLSGQYDVGNSSIQETKNKKRFLLLVVKIPKENRVLNKERVVGVDLGINTPLYAALNDNKYGGLSIGSRDQFLKVRMRMTAQKRELQRNLRHTTNGGHGRTQKLQALDRLEGKERNWVHLQNHIFSKSIIEYALQNDAGVIQMERLTGFGHDNNDEVDEKFKFILRYWSFFELQTMIEYKAKAAGIEVRYINPYHTSQTCNFCGHYEKGQRINQATFVCKNPDCIKGKGKQHSDGSFAGINADWNAARNIALSNDVVDKKKK

SEQ ID NO: 27 - protein, 488 aa, unidentified
MTAEYTCITRKIEVHLHKHGESEEATQRFKDEYRIWDDINNNLYKAANRIISHCFFNDAYEYRLKLQSPRFQEIEKLLGNTKRNKLSAEDIKVLKAERKLLFSDFKKQRQIFLRGGVEEGPNPEQNSTYKVVSQEFIDVIPSEVLTNLNQNISSIYREYALDVERGIRAIPNYKKGIPVPFSIKQKGEIVLKKREDGSIYVRFPKGLEWDLNFGRDRSNNREIVERVLNGQYDAGNSSIQETKNKKRFLLLVVKIPKESRSLNKERIVGVDLGINVPLCAALNDNEYGGISIGSRDQFLKVRMRMAAQKRELQHNLRHTTTGGHGRTQKLQALDRLEGRERNWVHLQNHIFSKTIIEYALKNNAGVIQMERLTGFGRDGKEEVQNEYKFILRYWSFFELQTMIEYKAKAVGIEVRYINPYHTSQTCSFCGHYEKEQRISQTTFVCKNPKCTKGKGKLKSDGTFEGINADWNASRNIAKSTEFVDKKKK

SEQ ID NO: 28 - protein, 487 aa, unidentified
MATEYTCITRKIEVHLHKHGDSEEATQRFKDEFRIWDDINNNLYKAANRIITHCFFNDAYEYRLKLHSPRLQEIEKLLSNSKRNKLSDEEVKQLKAERKQLFADFKKQRQVFLRGGVEAGANPEQNSTYKVVSNEFIDTIPSEVLTNLNQNISSTYREYSLDVERGIRTIPNYKKGIPVPFSIKQKGEIVLKKREDGSIYVRFPKGLEWDLSFGRDRSNNREIVERVLSGQYDVGNSSIQETNNKKFLLLVVKIPKQMASVDPNRIVGVDLGINVPLYAALNDNEYGGMGIGSRDQFLKVRMRMAAQRRELQRNLRYTTNGGHGRTQKLQALDRLEGKERNWVHLQNHIFSKSIIEYAVRNNAGIIQMERLTGFGRDENDEVGTDFKFLLRYWSFFELQSMIEYKAKAANIDVRYINPYHTSQTCSFCGHYEKGQRINQSTFVCKNPECAKGKGKQRADGTFEGINADWNAARNIAFSTEVVDKKKK

SEQ ID NO: 29 - protein, 488 aa, unidentified
MATEYTCITRKIEVHLHRHGDSDEAIQRYKDEFHIWDEINNNLYKVANRIISHCFFNDTYDYRLKLHSPRFQEIEKLLRNPKRNKLSGEDVKRLKAERKALDADFKKQRQAFLRGGVEEGTNKEQTSTYIVVSHEFIDIIPSEILTNLNKNIFSTYKKYRLDVEKGARTIPNYKKGIPVPISIKRSGELMLKKREDGSIYVRFPKGLEWDLFFGRDRSNNREIVERVLNGQYDVGISTIQETKNKKRFLLLVVKIPKESKNLNPNRVVGVDLGINIPLYAALNDNEYGGLGIGSREQFLKVRMRMAAQKRELQRNLRHTTNGGHGRAQKLQALDRLEGKERNWVHLQNHIFSKSIIEYALRNGAGVIQMERLAGFGRDKNEEVENEFKFILRYWSFFELQTMIEYKANAAGIEVRYIDPYHTSQTCSFCGHYEKGQRINQSTFVCKNPDCVKGKGKQHADGSYDGINADWNAARNIALSTTVVDKKKK

SEQ ID NO: 30 - protein, 488 aa, unidentified
MATEYITKTRKIEVYLHRHGDSDEAKQRYQQEWQIWHDINDNLYKVANRIMTHHFLNDEFVSRLRSTNPRYVEIEKILKHCKRNKLSQEEINSLQQENRALDALFTEKKNEFLGTTQEHNTILRIVRKEFGDVIPNDVYDCVIAERVKYTDKQKHLQIINGESSVPNYRKGMPVPFRIKIGANHNTLGILRRNDNPNHIYVKFPKGLEWDLVFGKDPSNNRKIVERILSGQYDAGNSSIQQAKNGKCFLLLVVKIPKSNIQLNKDRVVGVDLGINIPLYAALNDNIHSRLSIGSREQFLKMRMRMYAQKRELQRNLRHSTNGGHGRKQKLQALERLEGKERNWVHLQNHIFSKSVIEFAQKHNAGVIQMERLTGYGKDANGEMREEAKFLTRYWSYFELQTMIEYKANAAGIEIRYIDPYHTSQTCSFCGHYEKGQRVSQSTFICQNPECKQGKGKQKSDGTFEGINADWNAARNIALSTQYVDKKKK

SEQ ID NO: 31 - protein, 484 aa, unidentified
MATEYTCITRKIEVHLHRHGEDEDAVQRYKNEFQIWNEINNNLYKVANFISSHLFFNDAFVDRLRVQSNEYRDLLDLISKTTDAKEIKALENRKKALDAEFKRQQKIFLKGGSEDEKGSEKTAIRRIAVETFPNIPYSIINSLNDQISKTYNSSRFDVSIGKRTVPNYKKGIPVPFLMANGSGKIALREREDGSPYVLFPRGLEWDLHFGKDSSNNREIVKRVFNGEYKACDSSLQQAKNKKIFLSLVVKIPKKNHNLNPDRIVGVDLGINIPLYAALNDNDYGGMGIGSREQFLKVRMRMSAQKRELQRNLRQSTNGGHGRAQKLQALERLEGKERNWVHLQNHIFSKSIIEYALKNNAGAIQMERLTGFGRDKNDEVDSNFKFILRYWSFYELQTMIEYKANAAGIEVRYVDPYHTSQTCSFCGHYEKGQRLNQSTFVCKNPDCEKGKGKKLSDGTYQGINADWNAARNIALSDKIVDRKKK

SEQ ID NO: 32 - protein, 497 aa, unidentified
MNDSPVIKNKRHVKVLRLRILKPVSGTWQDLAKLLRDTRYRVYRLANLAVSEAYLGFHMWRTGRAETYELDTPGALNRRLRRMLDEEGVRADELDRFSKTGALPDTVVGALSQYKIRAATGKSKWQEVIRGKSSLPTYRLDMAIPLRCDKRNHARLARVENGDVTLDLMLCLRPYPRVVIQTGNIGGGAQAVLDRLLANPSQNPDGYRQRLFEIKHDDRDNKWWLYITYDFPAADPPRSSADRIVGVDIGVSCPIYVAINDGHARLGRRQFSSLGARIRSLQNQIVARRRSMQAGGKVALSGQTSRSGHGRKRKLRPIQKLEGRISHAYTTLNHQLSSSVIDFALSHGARVIQMEDLASLKDALRGTFIGARWRYHQLQQFLEYKAKESGLTLRKINPQFTSRRCSRCGFIHVEFDRARRDASRRDGYVARFVCPAPKCGFEADPDYNAARNIATPDIEKLISDQCKIQSIPTRSLTDQSEAADKDTLAQGQSRSGG

SEQ ID NO: 33 - protein, 492 aa, unidentified
MSEKRHNKVAKFQILKPAAGTTWPELANLLFAVRYRVFRLANLCISEHYLHYHLWRMGKTEEIPKLKISELNKKLREMIIEENDKKEKQNKINQDAINKKGALTSYVVDTLSQNKLGAVTSKSKWKEVILGKASLPTFRLNMAIPVRCDKPEQCRLKINANGDVELELMICERPRPRIILKTGGLSGSMKSVLDRLLENSAQSMEGYRQRNYEIIQDRNDGKKWYLHVSYDFPATERKPNSEIIVGVDVGFALPLYAALGNGHARLGWKQFHSLAKRIRSLQNQVVSRRRKMLRGGKDSLTQDTARSGHGRNRLLQPIEKLAGRIEKAYTTLNHQLSRSVVDFAKNHGAGIIQMEDLEGMKDAINGTFLGERWRYFELRQFIEYKAKEAGIEVRLANPKYTSRRCSACGYINMAFTREYRDSHRKNGKSAEFFCPECDKLPADDEQPQKPYPTDADYNAAKNLAALDIEKIIRRQCEKQGISYDKSPENNDL

SEQ ID NO: 34 - protein, 489 aa, unidentified
MANTEFTCITRKIEVHLHRHGDSDEAALRLKNEYHFWDEINDNLYKAANRIISHCFFNDTYEYRLKLHSPRFQEIEKLLKNAKRNKLTDEEIKELKAERKLLFSDFKKQRYTFLRGGIEDGANPEQNSTYRVVSNEFIDTIPSEVLTNLNQNISSTYKNYTIDVERGLRTIPNYKRGMPVPFSIKKHGVLALRKREDGSIYVAFPKGLEWDLSFGRDRSNNREIVERVLSGTYDVGNSSIQEAKNGKRFLLLVVKIPKEVKILDTSRVVGVDLGVAVPLYAAISDNEYGGMSIGSYDQFIKVRMRMNAQKREMQHNLRHTTNGGHGRKQKLHALERLEGKERNWVHLQNHIFSKSIIEYALQNNAGAIQMERLTGFGHDKNNEVDEGYKFILRYWSFFELQTMIEYKAKAAGIEVRYIDPYHTSQTCSFCGHYEKGQRLNQSKFVCKNPDCVKGKGKQRSDGSFEGINADWNAARNIALSTKIVDRKKK

SEQ ID NO: 35 - protein, 522 aa, unidentified
MENNITITRKYALIPEFSDRKEWKKRVYDFMINDLEQKIDYRNKKKQDTSELESQLEYIKNGGDFTRSMVNNYTYSLVRKAMEEETRRKNYILSWIFSEMRANRIDQMESLKDKFKFVSDTINYAYRKAGSNKGSLFDETEIHCILKSYGIAFSQELTKEIKELVKNGVLEGKVVIPTYKLDSPFTIAKSHFSFEHDYDSFEELCEHINDSDCKMYMNYGGDNRKDGINPASIARFRISLGHGKNKDELKSTLLKVYSGEYQYCGSSIQITKNKIILNLTMKIPKIETKLDENTVVGVDLGIAVPAMCALNNNMYERLAIGSADDFLRTRTKLQSQRRRLQKSLKNSNGGHGRNKKLKVLERLGKSETHFAETYCHMVSKRIVDFALKNNAKYINIENLNGYNTSSFILRNWSFYKLQQYITYKAERYGIVVRKINPCYTSQVCSVCGNWEDGQRKTQASFECANPKCESHKKYKYGFNADFNAARNIAMSTLFMEDGEVTEKKKEEAREYYGIKKENSEAV

SEQ ID NO: 36 - protein, 490 aa, unidentified
MATEYTCITRKIEVHLHKHGDGEEAEKRRAEEFRMWNEINDNLYKAANRIVSHCFFNDAYEYRLKIQSPRYKEIQRKLRYSKSNKLTDDEIKSLKAERKELDNEFRKQYRAFMLGGSSEGFKSTTEQNSTERIVNNEFGDIIPSNVLSCLNQNVFQTYKQYRTDVEFGKRTISNFKKGMPVPFSIKAHKSLMLKKREDGSIFVYFPKGLEWDLSFGRDRSNNREIVERILSGQYDAGTSSLQEGKNGKIFLLLVVKIPKQSNALDPNRVVGVDLGINIPLYAALNDNEYGGMSIGSREQFLKMRMRMVAQKRELQRNLRHSTNGGRGRSHKLQALERLEGKERNWVHLQNHIFSKNIIEFAVKNNAGVIQMERLTGFGHDRNDEVDDGFKFILRYWSFFELQSMIEYKAEAAGIEVRYIDPYHTSQTCSFCGHYEKGQRIDQATFVCKNPECEKGKGKKRSDGTYTGINADWNAARNIALSDKFVDKKKK

SEQ ID NO: 37 - protein, 490 aa, unidentified
MANDEICITRKIEVHLHLHGDDEEGRARRKKDFETWNTINDNLFKVANLIATHQFFNDAYEFRAKIHSPQYTKIEKDLNNSQKLKLTQEQVRELEKEKSKLDKEIEEQRKTFLQCSRQNSTFRVASKLFLDVIPSNVLTCLNQKICSTYTSYKSEVESGKRTLPNFKKGLPVPFQMNLNKKLQLRRRTDGSIFVLFPKGLEWDLFFGKDKSNNREIVNRVLSGEYGVGESSIQQNKKGKTFLLIVVKIPKKAICLDSKRVVGVDLGINVPLCAALNDNESAKMYIGSREQFMKVREQLYVRKRELQRSLTTSTRGGRGRKQKLQALERHEGKERNWTHLQNHIFSKSVIEFAQKQNAGVIQMENLSGFGRDKNDEVDEGYKYILRYWSYFELQQMIEYKAKASNIEVRYVDPKYTSQTCSYCGHYEKGQRISQSTFVCKNPECEKGKGKKTKDGKYEGINADWNAARNIALSGKVEERKKKKKRKIQSNQ

SEQ ID NO: 38 - protein, 576 aa, unidentified
MNTINNTYIRTLKFNLNLTPKFETEEDNKKYINDVYSYLRDAMWAQNRAMNIVLDRTKEAYTLGRGMNRVKEIYYSYSHQKPISNDKKESFLESLLQYAPIDDQFVKNEVKKLRKFYESKKKPPKEETVSKNCEALKNKYIKYVGKSKDDIKRELDLLENYCAYPEDIYEKFANGLSTPAYIKQKVESYWKQDGIKTKVIYSMDENLRRIKDAPLFIPPNIFYNKKDELIGLVYDYTDYISFLEDLENKRNVNIYLSIPYKKGEDKLKFKLVLGNPHKSRDSRLSIKRIFEEEYRIKGSSIGFTKNKETGKNTNLTLYLTVEVPQNKDNTLDENVVVGVDVGIAIPCVCALNNDKYTRENIGSYDTLFAKRTQFKMQRSRLNSQLKLSKGGHGRKRKLKKLELLSGKEKNYADTECRKYASDVIKFCLKNHAKYINLEHLKGYRENPKVLAGWSFYKIQTYIEQAAEKHGIIVRKINPCYTSQICSVCGNWHPENRPKGKLGQAYFNCHNIDCKTHNTDLYKYGINADFNAARNIAMSTLFITDSDEITKKHWKEAREYYGIDESDDKEEKLNKVA

SEQ ID NO: 39 - protein, 497 aa, unidentified
MIIARKLKITVIGSDEERKEKYRWIRDEQYNQYRGLNMGMTYLATGEILRMNESGLEIRLEKQKTELESKVEKAKLNIEKIKIKIEKIKTSKKINEEKILENQNKIDDEKANIIKYKNNIIKIEQALKVAKIKRMDIQMEFKEKYIDDLYQVLDKVPFQHLDNKSLITQRVKNDIKADKTSGLLKGERSIRNYKRTFPLLTRGRDLKFYYDDKDIKIKWIEGIEFKVVLGNRIKNSLELRHTLNKVVNEEYKICDSSLQFDKNNNLILNLTLDIPENNKNEKVEGRVVGVDLGMKIPAYVVLNDVEYIKKSIGSIDDFLKVRTQMQSRRRKFQKQLQSANGGRGRNKKLQSLSRFEEKEKKFAKTYNHFISSNIIKFAVDNKASQINLEFLSLKETQEKSVLRNWSYYQLQQFIEYKAKREGIGIKYVDPYHTSQTCSVCGNYEEGQREIQEKFICKNPKCKCELNADYNAARNIAKSTKYIKSKEESEFYKLNKKE

SEQ ID NO: 40 - protein, 427 aa, unidentified
MTKVVKLALICEQNDKDNNPIDYKEIYKILFELQRQTREIKNKSIQYCWEYSNFSSDYYKLNHEYPKEKEILSYTLDGFVNDKFKNGNDLYSGNCSTTVRSACGEFKNSRSDFLKGTKSVINYKGNQPLDLHNKAIRFDCIGKEYYVYLKLLNRPAFKKHNYANTEIRFKVLLYDNSSKTIVERCIDKIYKISASKLIYNEKKKCWMLNLSYSFSNDSETELDKDKILGVDLGIHYPICASINGERKFFKIDGGEIEHTRRKIEARKKSLLKQGISCGEGRIGHGIKTRNKPVYDIEDKISRFRDTANHKYSRALINYAVNNNCGIIQMEDLTGITSDSNRFLKNWSYFDLQTKIEYKAQEVGIKVVYIDPHYTSQRCSKCGYISKDNRTEQALFWCQKCGYKTNADYNASQNIAIKDIDKIINAKK

SEQ ID NO: 41 - protein, 482 aa, unidentified
MLKIKIECLNREVKNIITTRALKLTIVGDEETRNKQYKYIRNEQYEQYKALNLCMSLLNTHYVLNSYNTGAENKLKNQLEKLQNKIDKNNLELEKEDIKNSKKEKLTKQNMQFKGELIKLQEEYNKASKYRSDVDLAMKDMYIDDLYMAIQNQVTFKNKDFMSLVTQRAKKDFKNCLINGLARGERSLTNYKRNFPLMTRGERWLKFRYKEESDDILIDWIQGITFKVILGSRKNENTTELRHTLHKVITGEYKICDSEMKFDKSNNLMLNLTMDIPVKENTNYIDGRVLGVDLGIKYPAYVCLSDDTYKRMAIGSAQDFIRVREQIRTRRFRLQEQLKMVKGGKGREKKLGALERIKDKERGFVKTYNHMVSKNIVEFAYKNKCEYIHVEDLNKNGFDNAILSKWSYYELKTMIEYKAERKGIKVRYVNPEYTSQKCSKCGHTDKENRQSQEKFKCLNCGFELNADHNASINIARSNDIKK

SEQ ID NO: 42 - protein, 474 aa, unidentified
MADFVITRRIEVHLHHDPVADPEKVEYNRQWEYWRTINNNLYLAANRISSHLFFTDEYEHRLRIQHPRYRDIERTLASVSKVKRMSKEEIAALRVERRTIEQELRAQTAAFLQTSRQNVTYRIASDEFGDLIPSDVLTNLNQNITSTYNEFKKQVVRGERTLSNYKKGIPVPFSMKKGEGLRRRDDGSYYVLFPGGLEWDLAFGRDRSNNRAIVERAFNGDYEVGNSSLQEKNRKVFLLLVVKIPAQELALDPNRIVGVDLGLNIPLYAALNDNEYGGLAIGSREQFMKVRERMSARRRELQRALRHSTQGGRGREHKLQALERFQAKERNWVHTQNHIFSRAVVEYAKQNSAGVIQMESLKSFGRDKEDHIEAGFRYVVRYWSYFELQTLIAEKAKREGIEVRMIDPYHTSQTCSFCGHYEKGQRISQGVFICKNPECAKGKGRQLKDGTYSGINADWNAARNIALSTEVVKK

SEQ ID NO: 43 - protein, 472 aa, unidentified
MITARKIQVTIVSNNRDEDYRFIRNEMREQNKALNVGMNHLYFNYIARQKLRLADLAYQEKESKLVSQIDKIYDDIKKAKTDEKREQLKEKLEKQKKKLEKMRKQKNNDLFQQYQQIIGTSEQTSVRDAISEQFNLMSDTKDRLSQKVTQDFKNDIKAGLLTGERVLRTYKKDNPLYIRGRSLNLCKEEDTFYFKWIKGIVFQCVLGIKGQNKTELYKTLERVLDGNYKICDSALQFNKNNKLILILTLDIPDAVRESKIEGRVVGVDLGLKIPAYCSLNDDKYPRLAIGDIKDFLKVRTSLQRQYRSLQRALKSSKGGKGRYKKLKALDRFREKEKNYVTTYNHFLSREIVKFARKYRAEQINLELLSMAESTNKSVLRNWSYYQLQQFIEYKAAREGIKVKYVDPYRTSKTCSECGHFEEGQRTDQAHFTCKQCGFEANADYNAARNVAKSTKFITTKEQSEYYQKDSVS

SEQ ID NO: 44 - protein, 458 aa, unidentified
MNKVIKIYASLDKAQQCALPIYGKDSLSVQLLKVQSEVRSLKNRAMRMSYDYDQAQYEYFKYMERCKQEYGLNSYPSAQPSDFSKYKTFDGYLYDALAKDYPLMNKRISATVTRKVWGEYKKDKGEILSGKKSLRTYREGQPIPIRAKDTKLLYEDNFDYTMTVSVFSKDAAKTLGMKPGGCRFILHEQTDSEKAILDRLLSGEYKLCETLLAYDDRKNHDTKMARGWYFCIGYSFEKDTSNPSLDKDKILGVDIGVANVIYLGWSKDDHFKKYIPGSEIRKFQATEERRKKDILRCSVARGDGNVGHGRKCATRKAEKHEHHIHNFKETKNWHYAHFVVDTAVENGFGTIQMEDLSGINKSETFDRTWTFYSLQQKIEQLAAENGIVVKKVKPQYTSQMCSKCGYISSRSRRSQSEFRCVSCDYRRNADHNAAMNISKSKIEALVEEQLLRQGGEAD

SEQ ID NO: 45 - protein, 482 aa, unidentified
MYVEFIRIKGGEILITARKIKLTIAENREEGYSFIRKELQEQNKALNMAINHLYFNYVAREKIKLADETYKVKLEEGECYLERKYIELKEAKTDKQKENIKKSIEATKKKLETLRKVENKEVSNNFKEIIATSEQINLRDLISNNFNLKSDTKDKLTQKVVQDFYNDIVGVLRGERTLRRYKKDNPLYIRGRSLTLYREGEDYYIKWMNGIVFKCVLGVKKQNSLELQKTLDKVIFEEYKLCDSSIGFKDNKLILNLTLDIPVSNTNKFEKVIGRIVGVDLGMKIPAYCALNDSEDVRKAIGSIDDFLKVRTQMRSRRRKLQRALKSTNGGQGKNKKLSALNSFEAKEKNFAKTYNHFLSSNIIKFATDNKAEQINMEFLSLSETQNKSVLSNWSYYQLQQMIEYKAERIGIKVKYVDPYLTSQTCSECGHYEDGQREVQSEFQCKKCGCKTNADYNASRNIAKSDKYITKKEESEYYKNKI

SEQ ID NO: 46 - protein, 391 aa, unidentified
MRDKLKASFAGEKLKKIKNAQISSPSATSDRFPIPMWQQTGFRVETNNEDLVIDIPFPLYKYREEVDKFKPWEKLEFIDTSKKKHIQLILSTNSRKRNVGWVKDWSTEAEIKRVMSKKLVINKIEITRGKRINERNYWFVNFVIAYEKPVRKLDSKITGGIDVGVSNPVVCAINSGLNRLTIRDNDVVDFTRAELARLRSQRRGDRFRRGGHGIKHKFKPSETIQKKYEQRRKKKMEEWASRITRFFLNNGVGLVYMENIDKKSISDGEDYFKVQLRVTWPVKEMQKLFERKLKENGIEVKSIDPKYSSQLCSKCGKWNTHFNFRYRQLNGYPPFECKYCDYGKDKEKEIIYADYNAARNLANPNFDKRRKVAIPSEVLKGNIKEEVVAEN

SEQ ID NO: 47 - protein, 467 aa, unidentified
MITVRKLKLTIVGDEETRNQQYKLIRDEQYQQYRALNLCMSLLSTYNILNNWNSGAENKLNSQIEKLNKKVEKNKNDLKKDNLKENRIKKINESIQTLTKEKEKLQQEYLSSSEYRSDIDKKVKEMYIDDLYTVVQSQVNFKSKDMMSLVTQRAKKDFTTALKNGMAKGERSLTNYKRDFPLMTRGERWLKFEYDEDSDDIYINWLHGIRFKVVLGYKKNENSIELRHTLHKVINKEYKICDSSMQFDRNNNLILNLTLDIPLNIKNEHIEGRTLGVDLGIKYPAYVCLSDDTYKRKSIGCAEDFIKFREQIRARRYRLQKQLSMVKGGKGRNKKLQALDRIKDKERNFVKTYNHMISKNVVEFAKNHKCESINLEKLTKYGFPNMILSKWSYYELQNMIEYKAEREGISVKYVDPAYTSQTCSKCGHVDKENRTSQEKFKCIECGFELNADHNAAINIARSNNYVK

SEQ ID NO: 48 - protein, 441 aa, unidentified
MPQGKKLVTAQIKLYLHRPVEPDNISWAQAGQILRDVSYETVLGLNHAITEWHLYERERARHYRETKENLPAEERKKTWNRIYSEVRGLMKTASSGMASMVTRTAITRYQNSLKDIRSLKQSVPSYRLGHPILLREGGGETKLWRDNGNYMFRATLRNRSNEPTRLTFLLDTFKLEKSKKAVLDRVISGEYKLGACQVAQDRRKRWFTRIAYSFPRPELKKDTSICVGVDLGLACPFYCAVNNGHDRLSCNEALIVERFRWQIRRRRRAFQNSLKFSNRGGHGRNKALAPLEKLAEKEINFRDTKYHQYTSRIIEFALKQNAGVIQIEGLEGFRASQQGILRDWAIADFHSKLKYKAEHAGIEVREIDARYTSQRCSECGNINEANRQSQSDFLCTVCGYKTHADYNAARNISIVGIEKIIAEEIARKGLAEQEAQDVPQG

SEQ ID NO: 49 - protein, 411 aa, unidentified
MVKRGEHMNTVRKIKIIINNENNELRKKQYKFIRDSQYAQYQGLNRCMGYLMSGFYVNNMEIKSEEFKTWQKGVTNSANFFQEISFGKGIDSKSSITQKVKKDFSTALKNGLAKGERNINNYKRTFPLMTRGRDLKFKYDDNELDILINWVNKIQFKCVLGEHKNSLELQHTLHKVINNEYKIGQSSLYFNKKNELILILTIDIPTAKSSYEPIKDRILGVDLGMAVPVYMSINDNSYIKKSLGSYSEFAKVRKQFKERRNRLYKQLEACKGGRGRKDKLKAMNQFKEKEKNFAKTYNHFLSKNVVEFALKNKCEFIHLEKIESKGLENSVLENWTYYDLQEKIIYKAKREGIEIKFVNSSYTSQTCSKCNYVDKENRKTQVKFICKNCGFKANADYNASQNISKSKEFIK

SEQ ID NO: 50 - protein, 402 aa, unidentified
MITVRKLKLTIINDDETKRNEQYKFIRDSQYAQYQGLNLAMSVLTNAYLSANRDIKSDLFKETQKNLKNSSSIFNDIPFGKGIDSKSSITQKVKQDFSIAIKNGLAGGERNITNYKRTFPLMTRGRDLKFSYKDDCSDEIIIKWVNKIVFKVVIGRKDKNYLELMHTLNKVINGEYKVGQSSIYFDKSNKLILNLTLYIPEKKDDNSIKGRTLGVDLGIKYPAYVCLNDDTFIRQHIGESLELSKQREQFRNRRKRLQQQLKNVKGGKGREKKLAALDKVAVCERNFVKTYNHTISKRIIDFAKKNKCEFINLEQLTKDGFDNIILSNWSYYELQNMIKYKADREGIKVRYVNPAYTSQKCSKCGYIDKENRPTQEKFKCIKCGFELNADHNAAINISRLEE

SEQ ID NO: 51 - protein, 404 aa, unidentified
MNTVRKIKIIINNGDDEIRKSQYQFIRNAMYAQYQGLNRCMGYLMSGYYANNMDIKSQGFKDHQKTITNSLYIFNDIEFGKGIDSKSSITQKVKKDFSTALKNGLAKGERTVTNYKRSFPLMTRGRDLKFSYGDNDEILINWVNKIQFKAITGNSKNSIELEHTLHKIINGDYKVGQSSLTFNKKNELILILTIAIPEVKGDEYKPVANRTLGVDLGLAFPVYMALNDITYIRKSLGSYNEFAKQKLQYKARRERLYKQLDSVKGGKGRKDKLKALDQFKEKEKNFAKTYNHFLSKKIVLFAIKNQCEYINLEKIDSSGLENRVLGLWTYYDLQKDIEYKAKLAGIKVRYVKAAYTSQRCSRCGDIEKENRQEQSKFVCKKCGLDINADYNASINIAQSTEFIK

SEQ ID NO: 52 - protein, 428 aa, unidentified
MGNINIGGENMKEFRTVKKLKLTIVADTKEEREEKYKFIRDSQYAQYQGLNLAMGILVSGFLKGNRKLDSEAFKQAQKEMMAIREQTFEDINFGKGITSSSLITQKVKADFKTALKNGLAKGERNVTNYKRTFPLMIKGNADSCYDQGKRKPLDFYYDNDDIYIRWCNGIIFKVVLGTRINENTTELKHTLHKIIKGEYRVSQSSLQFDKNNNLIMNVNIRFKKELNTDFIEGRTLGVDLGLKYPAYVCLSDNTYIRKGLGSAEEFLKTRQQMKKRRTTLQHQLKLVKGGKGRNKKLKALEQFQNKERNFAKTYNHQLSCNIVKFAKENKCEFINLEKLTKEGFDNNILSSWSYYELQNMIEYKAERENIKVRYINPAYTSQKCSKCGYIDKENRKTQSEFNCLECGLKLNADHNAAINIANSTEYIK

SEQ ID NO: 53 - protein, 400 aa, unidentified
MITVRKVKLIVNSEEAEEINRTYKFIRDSMYAQYQGLNRCMGYLLSGYYANGMDIKSDGFKNHMKTIKNSLNIFDDINFGIGIDSKSAITQKVKKDFSTSLKNGLAKGERGATNYKRNFPLMTRGRDVKISYLEDTNTFVIKWVNKIEFKVILGQKDNIELSHTLHKIINKEYTLGQCTFEFDKNNKLLLALNINIPDNLISKNKEIIPGRVLGVDLGVKVPAMICLNDNTFIKKSIGSYNEFFKVRSQFKARRERLYKQLESSNGGKGRKHKLKATMQFRDKEKNFARTYNHFLSKNIIEFAQKYTCETINLEELNKKGFDNNLLGKWGYYQLQSMIEYKAERVGIKVKYVDPAFTSQTCSKCGYVDEENRITQDKFECQKCGFTLNADHNAAINIARK

SEQ ID NO: 54 - protein, 442 aa, unidentified
MPNITRKYQLKVVGDKEEIDRVYKYIREGTEAQNKALNEAMSALYAANLLDMSKDDKKELSKLFSRVTNGKNESGFTDDICFATGLGTTSSIKQKVKQDFNNACKKGLMYGRVSLPSYKADNPLLVSKSYVQLLSESDKNFGIYNTYETPMDLVDALEKETNPEVYLKFANNILFKFVFGNPWKGREQRKVFERIFSGEYKICGSSIGIDGKKIILNLCMDIPKQKHNLDESIIVGVDLGLAIPAMCALNNDDYKRLSIGSIDDLLRVRIQLQNERRRIQGNLKNSKGGHGRQKKLKALENLKDRERNFVQTYNHMVSKRVVDFAVKNNARYINIEDLSGFWKTRYGKSKSEDEKVLRNWSYYELQNYITYKAQLHGITVRKVRAEYTSQTCSYCGNKGIRKEQKKFVCVNPDCKCHKIYDGYINADFNAARNIAMSNDFAE

SEQ ID NO: 55 - protein, 428 aa, unidentified
MKLVKTMRYQIIKPLSCDWDTLGTVLRELQRDTHSVLNKTIQLCWEWQGYSSEYKAANGTYPTPKDTLGRSLEGYVYDRLKVQFPKMYTPNLSQTIQRAMLKWSADSKAIFKGEVSIPSYKKDVPLDLRKDSIHIERRGHDYILSLGLVSRAYKKELGLPECQIEVLIGTPDKTQRVILARLLTGEYTVSGSQIVWDKRNRKWFVNLAYHFEARPEQLDKTKILGVDLGVVFPVYMAVADGHFRAGIPGGEIEEFRRRVEARRRQLLRQGKYCGDGRIGHGRATRTRPLDKIADKIARFRDTINHKYSRYVVETARKLGCGVIQMEDLTGIREENLFLANWPYHDLQRKIEYKAREYGIEVRYVRPQYTSQRCSDCGYIHPDNRPEQAKFRCLACGFETNADYNAARNIATEGIEELIAAALNKASVV

SEQ ID NO: 56 - protein, 447 aa, unidentified
MVKTMKFQIIKPINMLWKDFENILRQLQQDTRNIKNKTIQLCWEYQGFSSEYKEKHGEYPKHSDILNYKTITGYIYDRLNNEYYRLNTGNLSDTIKSATDKWRNDIVDILKGEVSIPSYKRDAIIHVKNTNYKIVPEDGRYYLRLSLLSNVYRKELDLEKGQIDVAIRVADKNQKVTLERIFSGEYKQSSSQLMKKKNKWFFYMAFKFDPKTAKDLDPNNVMGIDMGITHPIYFAFNNSLKRGKIEGGEVESFRRRVEQRRRELLAQGKYCGKGRRGRGYETRVKPIKRIGDKIERFKDTANHKYSRYIVDVAVKNNCGIIQMEDLKGISKNNIFLKNWPYFDLQTKIEYKAKEKGIVVKKIAPRYTSQRCSKCGYINKENRVSQDTFKCVKCDFGHKFYVNADYNAARNIATPGIEEIIKKQIEKQKEMDEWENETIKLPLVGSDK

SEQ ID NO: 57 - protein, 439 aa, unidentified
MFMTKVTKVYLISEQIDKDGNKIDFKKISELLWNLQRQTRDIKNKCVQLCWEWLNFSSDYYKKSEEYPKEKDTLGYTLSGFVYDRIKNGSDLYSSNLSTSSRDTCTAFSNYKKEMLNGERSVLSFKANQPLDIHNKAIKLSYENGNFFVALKMLNRAGKEKYGINDDLRFRMQVRDKSVRTILERLMNDEYKVSASKLMYDKKKKLWKLNLCYSFDNHVISTLDPEKIMGVDLGVVYPIMASVNGDYARFSIKGGEIEAFRNRVEARRRSLLNQSRYCGDGRIGHGRKKRTEPAAQIADKIARFRDTTNHKYSRALIDYAIKNGCGTIQMEKLTGITSNAEHFLKEWSYFDLQTKIESKAKEAGIKVVYINPKFTSQRCNKCGYIHTDNRPVQARFCCQKCGYEENADYNASQNIGTKHIDVIIEETLKMQCEPEVPTE

SEQ ID NO: 58 - protein, 439 aa, unidentified
MKPMNKVVRLALICEHSDKDGNPVDYSDVYKLLWQLQAQTREIKNKTIQYCWEYSNFSSDYYKENHEYPKEKDVLHYALDGFVNDKFKVGNDLYSSNCSYMTRKVCAEFKKSKSDFLKGTRSIISYKSNQPLDLHNKSIRIEYKDNDFFAFLKLLKRPAFNRLGYKNSEIGFKVIVRDKSTRTILERCVDQIYGISASKLIYNKKKKQWFLNLVYAFEPDNANNLDPSKILGVDLGIHYPICASVYGDLQRFTIHGGEIEEFRRRVESRKLSLLKQGKNCGDGRIGHGGKTRNKPVYSIEDRIARFRDTVNHKYSRALIDYAVKKECGTIQMEDLSGITAESDRFLKNWSYYDLQSKIEYKAKEKGIKIVYIDPKYSSQRCSKCGHIDKENRKTQSSFVCLKCGFEENADYNASQNIGIKDIDKIIENDLSSKCETDVN

SEQ ID NO: 59 - protein, 433 aa, unidentified
MKITKTMKYEIDKSIDVPWKTFLSVLRDVQYAVWKTGNIAVKMTWDFQQEAWSYRQRFGEQLKFSDLGTGNKSQSTDIYQRACSEYPNVASSVLDATIRMAQDRYKTDAPDIYDGLKTIPYFKRELPIPIRAQQTKLTRKGTKRYVSFALLSKEGAKKSELPTRYNVQIRTGKGAREIFDRLVDGEYKLCDSKILRKKGKWYLALSYSFEAENPQELDPKRVMGVDLGIVKTAYMAFNFDEYLRYEIEGGEISAFRGRIESRRKSLLKQARYCGEGRRGHGRKTRMKPLEKLRDKVANFRRTKNHHYSKYIVEMAAKHGCGTIQMEDLSGINKRDKFLANWSYYELQSFVKYKAKERGIKVVLVDPNYTSQRCSCCGYIAEGNRKTQETFKCVICGYKTNADFNAARNIAEPRIKALIDAELKRQEKERKEAM

SEQ ID NO: 60 - protein, 427 aa, unidentified
MSKVMKYELKYLGEEDFYEMQKMLWSLQEDTREILNKTIQIFFHWDYTNKESLETTGKALNLVEETGYKDISGYVYDKLKSRYPDMSRGNLSATIRAASKKYRSSKVDILKGTMSIPSYKKDQPIILRPDGIRLHEREMIYTVELSLFSGDFKKKKAWKSNVLFQIKACDKAQKAIMQRLLSREYKLGESKLVYKKKKWFIYITYSFKKTDAKLDKNKILGVDLGVTYAIYACSIGEYGSFSIKGEEALEYAKRLEARTISKQKQARYCGEGRIGHGIKTRLSTVYSTRNKLANHRDTLNHRYSKAVVDYAVKNRYGTIQMENLSCIKKNTGFPKRLQHWTYYDLQSKIEYKAAEQGIQVIKINPKFTSLRCSQCGCIHKDNRKTQESFQCVECGYKDNADHNAALNISIPQIDLIIKEEMTSAKEK

SEQ ID NO: 61 - protein, 462 aa, unidentified
MPIKALRVQIIKPFNTDYDSQPITWDELGRTLRDLRYAASKMANYVIQQNYMWEFFRQQYKQEHGSYPSVSEHKDKLYCYPRLTAMFPLAAGQMVNQIERHAKTVWSARKSEVLKLHQSVPSFKLNFPIIVHHDSYRISEVPEDGKSSTHVFLLQANLLSREAATRTRYSFLINAGEKSKQTIVERIISGEYRQGALQIVGDRKNKWYCIIPYEFKTEENNTLDPQCIMGIDLGISKAVYWAIIGSHKRGWIDGHEIEEFRRRVQARRKSIQEQGKYCGDGRIGHGRKRRLLPIEVLENRESNFKNTTNHRYSRFIIEAAIKNQCGVIQMEDLSGINERSTFLRNWTYYDLQMKIKAKAEEVGIEVRIVNPQYTSQRCSQCGHIDRDNRSNQATFVCTHCGYGGLYHCFACGKSQVEAGVCHLCGGETKIMKINADYNAARNLAICGIDQIIVQTLEGEGVR

SEQ ID NO: 62 - protein, 427 aa, unidentified
MTQHIRVMQYQIVKPCNGDWSTLAKVLSYLQNATRQVLNKTIQLCWEWQGFGSDYKRKFGENAVDRELLGYSLFGFCYHQLRQEFPLIHSSNISQSIQRAVLRWNSDAPEILNGVKSVPCYKRGVGVDLHKDTVRLKRGKNNEFVVSMNLLSLIGRKEFGFKSAALDTVIKVADGRQRQIFSRVLSGEYGLGACQIVCHKRKWFLQMRYKLAVKERQLNSNHILAVRMGIERPLYIAFSHTSSRVTLNGDNVTAFRKKFMARYAGMIRQKTMQGGGNIGRGKAKRFERIESMRTKISGFQKSLNHKYSRIIVEQAVLNRCGTIQIESPNVIRRQTAFLGNWAYFDLKRKVEYKAQQLGIKVVCLKTRDYEQRCSQCGHLNAVRETSKVLSGAYARSFFCESCGLLTTVDENAVQNLTLPNKPVMSQD

SEQ ID NO: 63 - protein, 444 aa, unidentified
MALVKSVRIQISRCEELDYKRMSSIFRDIRYKTCRASNEAMRLFLLNAFQSIRYKELHDIYPDIKSLTGKSLNTYIYNSMKEIMDICQTGNVAQTQQFVKNRFNTDIKDLLVNEVSYTNFKKDMPLFLHNKSYTICKDEDSGKYKVECSLFNRQYQKENNIKRVTFVLGKMDGNQKATLDKIIAGEYKQGAGHIKQDKKKKWYFTISYSFEPQIRKINTNRILGVDLGMVNTAVLQIWDIDKQKWDWLEWKECMLDGGKVYNFNQRVEGMKRSLQRSRKVSSVTSPYSKGKKGRGVQARIKALNRLNHKISNFKDTINHQYSKYIIDFALKHNCGIIQMEDLSKIKDKAEEKFLAHWTYYDLQSKIGYKAKAHGIKVVKIKPAYTSLRCSKCGHIDKENRKEQAKFKCVKCKYKLHADVNAARNIAIPEIERLIEIEKEEVNAI

SEQ ID NO: 64 - protein, 447 aa, unidentified
MTKNKMTTKTMKYEIRFEKALYNLLSDIQFEVFMLKNKATSIAYDWQNFSFSYHSRFGEYPKIKELSGVTLTNDILGELREVQAKFVSSATVASSVKEAVEKFTSDKSKILKGEISIMRYKRDGSFPIRSQQINHLTKINSKTYTCKLSLLSREGAKEREMKNGQMDVELRTGKGAYEILDRIIEGSYKLCDSRITKNKNKFYLLITYSFESDKVETLDENRIMGIDLGINIPAMLAISDNKYYREAVGDVTEISNFQKQVESRKRKLQKQRKWCGEGSIGHGTKTRIKPLEVLSGKIKRFKDTKNHNWSRYIVDQAVKHNCGIIQMEDLSGIAEENTFLKTWTYYDLQQKIKYKAEEKGINVVFIKPNYTSQRCSCCGHISKENRDVEKNGQDKFICVNCGYGSKFYVNADWNAAKNIATKDIENIIKEQLESQEKELKHNMKYAM

SEQ ID NO: 65 - protein, 444 aa, unidentified
MDVGTVVKMTRCRIEQDDELYKVLDDLRYMIYRIKNKATSMAWDWEQFSFGYHERFGEYPVAKEVVGKNVSRDAYQHVKGLGEEFSSSFVDTAVEEASKHFKNHRSAILRGKEGIPVYRSDTSFTIRHTQIKGLKKEQRDKYSGLFTLLSPKGSKVREQASTRYLFKIRSGGSSSAILDRILEGTYSLCDSKIVYEKKKRHYFLLVTYKFEAQKIEMNPERVMGVDVGFAVPATIAITDNPYACHFVGDAREVLAFEQRVLGTRRALYRSRSRAGDGSRGHGRKTLMKPTEVINSKVANFKATKNHEWSKFIVDYAVRHGVSTIQLENLEKIAEESPFLKRWTYYDLQQKIAYKAKEYGIKVIRVSPDYTSARCNKCGAIHRKQERTLWRPKQSQFNCLHCHHRDHADRNAARNLAIPNIDQIIKAEKVEWTSYWTNVYKNPPA

SEQ ID NO: 66 - protein, 450 aa, unidentified
MESEKEKYVVQSYGIKLIKPVGVDEGDWDFAGKVLRDLDYACFRVKNKAATRTYMNVIEKLEYENIHGKGTYDKYFKTRYGKTFSAYNSECAREDKELNKGDLYREYFNLMAREGEKVVKYNMKKILNGNASNITFKRNQPVPITSRMIFITKESGKYYAELTLLSPEKAKELGRKGKNGTRIKFLLSSKGQEKVILDRLTSGEYDLRDSHIHVKKKGNKLTNYLIIAYRHKVKDDNDLIPNKVLGVDLGVSKAAYMAVSESPVSEYINGGEIEQFRNGVEARRNGMRNQLKYHSSNRSGHGRATKLKPLEKLREKVSNFRKLTNHRYAKFIVDTALKNNCSIIQMEELKGISKNDTFLKRWSYFDLQEKIENKAIAVGIEVKKVSPKFTSQRCNKCGYIDKESRKSQEKFECVNCGHKTNADLNAARNLSMLNVEKEIKAQCKAQKIKY

SEQ ID NO: 67 - protein, 426 aa, unidentified
MLQVTKAVRFQIIKPLNFSWDEFGRILNDLSYHTTLMCNAAVQMYWEHNVMRNRYKAEHGRYPQDKEIYGQSFRNVVYHRLREMYPLMASSNVSQTNQFALKRWQTDLREVMRLQKSVPSFRLGTPVQVANQNYSLYIAKGEPPEYCAEITLLGKDAACRRFTVLLDAGDAPKKAVFRRIVEGKYKQGVMQIIKHPRKKKWFCIVSYTITKDPAPGLDQERVMGVNLATGEAVYWAFSFSPKRGSIPAGEIEAAEKKIRAITARRREMQRTAGVTGHGRKRRLKATRVLAGKTANIRDAINHKYSRRIVRIAAANRCGKIRLADMSALGMSGALKAWPWSDLVQKIGYKAAEQGIDVEIVEKPGDRAKAWHTCSECGYSAPENVGDNTEFLACKECGARISLEYNAALNIAVLARDSIPEQQTSAS

SEQ ID NO: 68 - protein, 431 aa, unidentified
MEITKATRYQLIKPLDVSWEDFGQILRDLSYHTTKMCNAAVQLYWEYHNQRLAYKQEHGKYPEDKVMYGMSFRNVVYHRLRGIYPLMASSNTSQTNQFALNRWKNDVPDVMRLQKSIPSFRLGAPIQVANANYRLYVAEGEKPEFRADVTLLGKDAAQGRFSLLLDGGDAPKKAIFRHIVDGTYKQGVMQIVRHPRKKKWFCIISFTFTSEEKSLDESRAMGVNFGSGEALCWAFNFGPKRGTIPAAEIEAAEAKIAAITARRRGMLRTAEARGHGRTRRLKPTESLQGQAANIRDAINHKYSRKIVSVAVGNRCGIIRLADTSGLELDGAFKHWPWAGLAEKIRYKAEEAGIVVETADAKKAFYTCSKCGYSHPENTDGNKEFLTCKNPDCEAQVNLQYNTAKNIAVNVPGPEEKKSPKKEKRKKEIAKQ

SEQ ID NO: 69 - protein, 411 aa, unidentified
MTKAKDRELSKVFRVEVLKPLNLTWDEFGGLLRRTQYHAAHLANEVITTQYLLAKGKLERNGSFCALVAHACHDCSLHADVKCTVCKWARDKFKADARRILRADISLPSYKNNLCMIKNRSVKLRETPDGWAARLAILPKADGKNQVQPEVLLRTEEMKRRSPGAYQVLERIASKEYKQGTTQVKRDTRTGKIYLLISYSFRPERESGLEASRVMGVDLGVSTPAYCAFNDSLKRKSLLIEGRKLLKTKWQIEGRRRDIRRHNDQRDLRRGHGKEAKFRPMEAVEQHWVDFRQSWNHVLARRIIEYALSNHAGAIHLEDLSPGTNSKFLGRNWPVAELLDYIEYKAKERGIAVKRVNPFKTSQTCSDCGAIKESFTFGDRKKAGFPDFVCDACGFRTHADYNAARNIAKAP

SEQ ID NO: 70 - protein, 495 aa, unidentified
MNKCIKVILNKCINIDIKEAKKIIKNMSYLSCKASNKAIDMWKQHSLNIMELKSNDKNFNQKEYEQATYGKNYKNVIEGYMKEIMNICNTSNVSTLHQQQVQNDWKRLRKDVLNYRANLPTYKLDTPCYLKNNNYKLRNHNGYFVDISLFSMKGLEQIGQKKGYQLQFEIDKMDGNKKSTINKLINNGYKQGSAQLKISDKGKIELIMSFSFEAKESNLDENRILGIDLGIVNVATMAIWDGNTQEWDWVNYKHNILNGQELIRFRQKLFNMGMSEFEMQNEVYKQNQKIHQKQLNKHNIGAIDGLELVKYRDTIDKKKREMSIASKWVGEGRVGHGYKNRMKPLEKIRNKASNFADTFNHKYSKYIVEFAIRGNCGVIQMEDLSGATKNTHGKFLKDWSYYDLQTKIEYKAREVGIDVIYVKPQYTSKRCSKCGNIHTDNRDCKTNQAKFKCMNVTCGHEENADINASKNISIPYIDKIIEEYIKENDIYKNKK

SEQ ID NO: 71 - protein, 433 aa, unidentified
MIQNRRQNRLILTRKIQIIPLGEKEEIDRVYKYLRDGIFYQNKAMNQYMSALYIAAIKDISKEDRKELNRLYSRVSNSKKGSAYDKSIEFAKNMNLGYVVKQVKQDFANSCKNGLLCGKVSLPTYRKNNPLLVHVNFVRLRSTNYHQDNGMYHNYESHTDFLDHLYSKDLEVFIKFANNITFKMIFGNPHKSAYLRSEIQQIFEENYKVCGSSIQIDGKKIILNLSMDIPKQELELDENIVVGVDLGLAIPAMCGLNTNDYIRQSIGSKDDFLRIRTQLQSQRRRLQKSLASTSGGHGRQKKLKPLEKLKDRERNFVKTYNHYVSKNVVDFAVKNKAKYINVEDLSGFDSNQFILRNWSFYELQQFITYKAAKYGIEVRKINPYHTSQICSCCGHWEEGQRIDQAHFKCKSCGAELNADFNASRNIAMSTDFV

SEQ ID NO: 72 - protein, 471 aa, unidentified
MATKEKRIVKVMRLRILKPAGEMSWSQLGKLLRDTRYRVFRLANLAVSEAYLNFHLWRTGRSQEFKADDMGKLSRRLRQMLADEGVAADELDRFSPTGAVPDAVSGPLFQYKIRAITNKNKWREVIRGTASLPTFRLDMAIPVRCDKARMRRLERMENGDVQVELTICRKPYPRVVLQTGDIGGGQEAILARLLDNTGNDLTGYRQRVFEIKQERQTSKWWLYITYDLPAPQTGKADPDVVVGVDVGYAVPLYVAINNGHARLGWRQFDALGRRIRKLQTQVLARRRSIQRGGRVNISHATARSGHGVKRKLLPTEILQRRIDKAYQTLNHQLSASVIDFARDHGAGVIQVEDLEGLKEELTGTYIGARWRYHQLHQFLKYKAEENGIEFRAVNPRFTSRRCSKCGHINVAFDRAYRDAHRENGKTARFICPQCGFEADADYNAARNLATLDIEALIEAQCARQGLTKDAL

SEQ ID NO: 73 - protein, 408 aa, unidentified
MITVRKLKLSIRESDEESRKVKYQFIRDSQYAQYRALNLAVGILSSAYLKSNKDTKQESYKNAIKSLTNSNPIFSEIEFGRGVDTLSLVTQRAKKDFKNSIKNGLARGERNLTSYKRTYPLMTRGRDLKFRYEGDDIVIRWVNKIEFNVITGSNKIKENTVELKHTLHKVINKEYNVKESSLMFDKNNNLILNLTLDIPNELSYQPIEGRTLGVDLGIATPAYVCLSDDTYVRKGIGSIEDFLKVRTQFQKRRRVLNQSLVMAKGGKGRKKKLKALESFKEKERNFAKTYNHQLSHEIVKFAKNHKCESINLEKLTKEGFNNRILRNWSYYELQSMIEYKAEREGIKVRYVNPAYTSQKCSKCGHVDKANRQTQAQFKCVECDFELHADHNASINIARSEEFVQGAGS

SEQ ID NO: 74 - protein, 411 aa, unidentified
MVKRGEHMNTVRKIKIIINNENNELRKEQYKFIRDSQYAQYQGLNRCMGYLMSGFYVNNMDIKSEEFKTWQKGVTNSANFFQEISFGKGIDSKSSITQKVKKDFSIALKNGLAKGERNINNYKRIAPLMTRGRNLKFKYDDNELDILINWVNKIQFKCVLGEHKNSLELQHTLHKVINNEYKIGQSSLYFNKKNELILILTIDIPTAKSSYEPIKDRILGVDLGMAVPVYMSINDNSYIKKSLGSYSEFAKVRKQFKERRNRLYKQLEACKGGRGRKDKLKAMNQFKEKEKNFAKTYNHFLSKNIVEFALKNKCEFIHLEKIESKGLENSVLANWTYYDLQEKIIYKAKREGIGIKFVNSSYTSQTCSKCNYVDKENRKTQAKFICKNCGFKANADYNASQNISKSKEFIK

SEQ ID NO: 75 - protein, 481 aa, unidentified
MITVRKIKLTIMGDEETRNRQYKWIKDEQYNQYKALNIGMSYLATHLFLKMSESGLEQKTEKNIKTIEKQISKIENNITKEESKKKVNEEKLNNLINELDTLNASLSKLKEELEEISNNRSNVDDTFKRMYVDDLYNALSKVPFQHSDMKSLVSRKVKLDFNTDMKDLMSGNRSVRNYKRNHPLLVRGRDLRFRYDGSNIKIKWIQGIEFKAILGKISKTIELRHILNKVIDGEYKVCDSSLEFNKNNHLILNLAIDFPYTNKIEFIEGRVVGVDLGIAVPAYVALNDIAYVKKSIGDIDDFLRVKTQMKKRRNLQINLTSVKGGKGRSKKLKALDRLSEKESNFVRTYNHFLSKSIVKFAIDNKAGQINLELLSENALSDKIIKNWSYYQLQQFIKYKAERYGIKVKYVDPYRTSQTCSVCGHYEEGQRSKQDIFTCKNEKCKMFEKEVNADYNAARNIAISTKYIDDIKESEYYYKKSF

SEQ ID NO: 76 - protein, 475 aa, unidentified
MSIPLTRKIKLSVYVPNTITEEKERHEYKTFVWQLLRKVNQQNYEFHNLLVKKLIELDSVLEGRLTEDDNFIEIRNRYYSDRKDKKNQREFFDYIRMKKDELIKEFGGKNLHGYIYTYLKNYIKSLPEEQQFMASYTYGSISKNVVDKYTNDQFDVFRGVKTIATYKNTQPIPINIIGHVKKNEKGELYKNTGKEWFNKYDDIYTFKFKSIKKHEIELQLNFGKDRSNNRIIVDRIYNNDPNYKICDSKIQVVGTEIFLLLVFKQFVTKRELGLDVRKVIGVDLGVKNVATIATNFSDHIEIIGEGEFVLMRTKLQIEKQKRSIQRNSKYSRGGHGRNRKLQKLNEYRNYERNFRSTFNHKISKDVIEIAIKHGAGQINIEDLSSIPLKEKNNRILRFWSYYDLIQKIIYKAKREGIIVNLINPSYTSQKCHQCGQIGNRPKQDTFLCTNPTCKAFNEPINADVNAAKNIAKISL

SEQ ID NO: 77 - protein, 419 aa, unidentified
MQRSITLKILRPRDEKISWEEMGYLLGGLSMKVCRMSNFCMTHHLLHALKLETELLNPRGDLYCYPVLAEEYPEVPSGIICAAETRARKLFKRSAAKVLRSETSLPSFRKDSSIPIPVAGYRILQDGDGNYCAEIQLISRQGAKTQKLPGRICLVLADNWRDKSAKSALQKVAAGKVRRGVATLFRAKKDWYLCIPYVTEPADIGENFEPGLVMGVAFGMFDVLAYGFNTLLKRGAISGEEVLSHQDKFMARRKKIQEQYAWSGRKGQGREDALKPLRHLYEVEKNYRDLVNNRYAKWVVDIAVKNRCGEIHLDSGNSTSKGNKEILLSHWSLYDLKDKICRKAEEKGIRVTECNVPNLRTRCSHCGTEQAVENRKRMFLCKNCGYGTTDKNKSNGYISADYNAARNLAVYDTGDTEPV

SEQ ID NO: 78 - protein, 481 aa, unidentified
MIITRKIAITIVSEEAQESYNYLRQQIYYYYKALNFGMNHIYFNYVAKEKIKLADSAYKEREEKYINAIHIAKEKLQKDLSVSQRAQAEKSLEVNTNNLDKLRKAISKDAKETFQKVMGAVERTNVTDAIKKEFPMLQRDSIDFAASKVASDFNNDLKLGLMTGSRTLRIYKRNQAYPFRSRRLKFYKENGDFFINSSKSLLFKCLLGVKRQNSKELIQILEKILDNQYKICDSSLEFNKKKLILNLCIEVDENTHSENMKVPGVVKGRIVGVDLGIQIPAYCTLNDSPFKKKAIGSVDDLLRIRTQMQARRRRLSKNLISARGGKGRGKKLKALDRFEEYERNYVRTYNHFISKQIIQFTLQNQAEQINLELLQMEHTKSKSILRNWSYYQLQQMIEYKAKREGIVIKYVDPYHTSQVCSKCGHFEENQRMDQNTFRCKKCKYRTNADYNAAKNIANSTRYISSIQESEYYNIKNRNIAK

SEQ ID NO: 79 - protein, 481 aa, unidentified
MIITRKIAITIVSEEAQESYNYLRQQIYYYYKALNFGMNHIYFNYVAKEKIKLADSAYKEREEKYINAIHIAKEKLQKDLSVSQRAQAEKSLEVNTNNLDKLRKAISKDAKETFQKVMGAVERTNVTDAIKKEFPMLQRDSIDFAASKVASDFNNDLKLGLMTGSRTLRIYKRNQAYPFRSRRLKFYKENGDFFINSSKSLLFKCLLGVKRQNSKELIQILEKILDNQYKICDSSLEFNKKKLILNLCIEVDENTHSENMKVPGVVKGRIVGVDLGIKIPAYCTLNDSPFKKKAIGSVDDLLRIRTQMQARRRRLSKNLISARGGKGRGKKLKALDRFEEYERNYVRTYNHFISKQIIQFTLQNQAEQINLELLQMEHTKSKSILRNWSYYQLQQMIEYKAKREGIVIKYVDPYHTSQVCSKCGHFEENQRMDQNTFRCKKCKYRTNADYNAAKNIANSTRYISSIQESEYYNIKNRNIAK

SEQ ID NO: 80 - protein, 531 aa, unidentified
MNDRSIITRKLTLIPAFSDRPKWEEKVMSYTEQFYIDKIAYYKNKLTKTKGKEEKQKIKDKLASLEEQENEFEESGILTQANVTDYTYDLVRNAMASEANRKNAIISYIILELLHNGGQTMDFNARNKLINDLVNYGLRVKGSSKGSLFDELDIENPLNAYGFAFKQDLKKKIRDMVNSKRVLDGKSSVITYKADSPFSINKENMSFTHDYSSFEELSDHIRDNDTNLYFNFGSSGNPTIARFKINLGAGRHKKNKDELIATLLKLYSGEYQFCGSRIGIEKNKIILNLVLSIPKKVRALDENTVVGVNLGVAVPAMCALNNNEYERLAIGSADEFLRVRTKLQAQRRRLQKSLKDASGGHGRTKKLKALERVAKAESHFANTYCHMISKRIVDFALKNNAKYINLENLTGYDTNDFILRNWSYYKMQQYTTYKAEKYGIIVRKVNPCYNAQACSVCGNYAPGQRKSRAVFICANPACKSHKKNHGKLDAEFNNARNVAMSTLYMNDGQVTEKSFKEARDYFGIEEEIETI

SEQ ID NO: 81 - protein, 471 aa, unidentified
MITARKVKLTITENREDGYNFIHNELREQNQALNMAMNHLYFNYVAREKIKLADETHKIKLAEDQGYLDQKYTELKEVKTDKKKQNIRKSIQAAKKRLETLRKAENKQVAEKFKEIIAASEKTNLRDFITDNFNLTSDTKDRLTQKVSADFKNDIVDVLRGERTLRRYKKGNPLYIRGRNLTFYIKDEEYYIKWMKSIVFKCVLGVKKQNSLELQKTLDKVIEGKYKVCDSSIEFKQNSLILNLTLNIPVCNSFDKVEGRVVGVDLGMKIPAYVTLNDSDYIRRAIGSIDDFLKVRTQMQSRRRNLQRALKSTKGGKGREKKLKALNQFEVKEKNFAKTYNNFISSNIVKFASDNKAKQINMEFLSLSETQNKSVLRNWSYYQLQQMIEYKANRVGIKVKYVDPYHTSQICSKCGHYEEGQREKQEVFICKNPECKNFNIEVNADYNASRNIAKSNKYITKKEESEYYKIN

SEQ ID NO: 82 - protein, 496 aa, unidentified
MILTRKIKLVIVSENREEGYNLIRTEIREQHKALNLAYNHLYFEHNAIQKLKQNDEDYKQKRNKLQELINKKYEEHQKAKNLEKKEALREAYNNKKQELYNFEKEYNEKARQTYQQVVGFTQQTRVRNLINRECNLMSDTKDGITSKVTQDYKNDCKAGLLIGKRSLRNYKKDNPLLVRGRSLKFYKEDGDYFIKWNKGTIFKCILHIRKKNVVELQSVLENVLLGAYKVCDSSIGFNNKDMILNLSLNIPDKETQGYIPGRVVGVDLGLKIPAYLSLSDKVYVRKGIGSIDDFLRVRTQMQKRRRRLQKSLAAVKGGKGREKKLKALDHLKGKEANFAKTYNHFLSTQIVTFAVKNQAGQINMEFLEFDKMKNKSLLRNWSYYQLQIMVEYKAKREGIIIKYVDAYLTSQTCSKCDHYEDGQREKQENFMCKNCGLEVNADYNASQNIAKSTSYISDSTESEYHKKKQQVLKEILGENDIMNEQLSLFNNCDDIA

SEQ ID NO: 83 - protein, 453 aa, unidentified
MSKITRKIKIIPDIDGITHEESNKKCYNTFYKFDRKLYKVANLLVSQLYGLDSLLSLMRLQNDEYVKCLSKLSFKSITDATKEEIKKRMKEIDAELISIKNDIAPKHPQTYSYRAVTSSEYAKDIPSDILNNLKQDVYQHFNENKKEQIRGERSLATYKKGMPIPFNLKKKHGIISVGDNYYLPWFEDTRFRLNFGRDRSNNRAIIDNCIKTKKYKLCAAAKIQLKERKLFLLITVDIPKAESVPVKGKVMGVDLGVVNPAYVAVNDGPERSRIGNGEAFQKQRDVFRRRFRELQRSQLTQGGHGRKHKTKATEILRGKERNWVQTENHRISREIVNLASRWKVETIQMESLKGFGKNQEGEVEYNHKRLLGRWSYFELQKDIEYKAAMAGIAVQYVNPAYTSQTCHVCGQRGNRIERDTFICTNPECTCYNQAQDADMNAAINIAKSKDVIK

SEQ ID NO: 84 - protein, 415 aa, unidentified
MITVRKLKILIDGESRNESYKFIRDSMYAQYLALNKAMSYLGTAYLSRDKEIFKEAIKSLNNSNPIFDNINFGKGIDTKSSVNQTVKKHIQADIKNGLAKGERSIRNYKRDYPLMTRGRDLKFFYCDTNSTKVKVKWVNGIIFDVMLGKEYNKNDLELRSFLNRVINKEYKISQSSICFDKHNRLILNLSVNITDNIPNEVVKGRIVGVDLGMKIPAYVTLNDSEYIGKPIGDINDFLKVRKQFKERKERLQKQLAINKGGRGITNKMQLMDAFTNKEKNFANTYNHGVSKAIINFAKKYKAEQINVEFLALAGSEKEILSSTIRYWSYYQLQQMIEYKANREGIAVKYVDPYLTSQTCCKCGNYEVGQRINQELFECKLCGNKMNADRNASFNIARSTKYISSKEESDFYKQLK

SEQ ID NO: 85 - protein, 417 aa, unidentified
MQRSVTLKIIRPEDETISWEELGYLLRGLSFKVCRMCNFCMTHQLLHALKLETELLNPQGNLYCYPRLAEEYPDVPTGIICAAETRARKVFRRSAEAVLHSETSLPRFRKDSSIPVPVAGYKILQDSDHNVYADVQLLSRQGAKTQKLPGRIRLVLADNWRDQSAKAALRQIADGKVKRGVASLFRVKNDWYFQIPYVTEAVNTGEGFEPDLVMGVAFGLQDALVYAFNTSLKRGAVSGEEVLAHQEKYAARRKKIQEQYNWSGRKGHGREDALKPLRHLYETERNYRSLVNSRYAKWVVDIAMKNRCGMIHLDSANYVSSGKKILLSRWPLYDLKEKIRRKAEEKGIQVTECSIPNLRTRCSLCGKEQEPEGEKHTFVCKDCGYGKADKNRRSGSITVDYNAARNLAAYKSEDTKL

SEQ ID NO: 86 - protein, 400 aa, unidentified
MITVKLQLYKPTKCKEQRLFSIIREFTSCANWYLDKLQESRTTSRVKIHNGYYETARKSFRLLSANVQLALDKAIETQRAFLNKKGKKSVPKFKKQFACFRQDTFKIFDRYVQFNMPGRERVNIPFKVCNPNHKQFIKQQPKRSQLINKKGKWFLYVSYETDKPAIDGNINIIGVDLGVKKIVTVSNPEASVNVFFSGNKAIYTRNKYQRYRKQIQRAKDTGKAKRGYRALKRISGKEKNWIKDTNHKISKQIVNIAKQNKADIAIENLKGIRERIKATKKVRRMLHSWSFRQLISFLQYKSAMAGVRIVSVDPRHTSQRCPRCGHISKDNRKSQSAFKCSQCHYSVNADLVGSRNIALTALNLYGEGKRPSERAVMLLPMAEGKTLMASCLEAPSVRAG

SEQ ID NO: 87 - protein, 400 aa, unidentified
MPTITLRLELHNPTKVKQDMYERMTEVNTAFTNWLLNHPKLNQATSKIFKEFSPQRFPSAVVNQTIREVKSQKKNQKTKKFQTLWCCFNNQNVKVEKKGSFYTVSFPTLEKRIGVPVVTRPYQEAWLNRLLDGTAKQGAAKLYKKRKKWYLAIAITFDVKPRHETKVMGVDVGLRYIAVASVGTKSLFFKGSQCAFIRRRYAALRRTLGKAKKLQMIRKIGRKESRWMKDQNHKISRQIVNVALANGVGVIRMEALTGIRKRAKSAKEAGRSLHAWAFHQLQTMIAYKAEMAGIRVEWVDPTYTSQTCKCGHREKANRNGIRFRCQRCGYTLHADLNGAINIAKAISGFAAEPSALVTGAPPIGVHRNPMGRGDDTPLKLLRCPNQKWMRTQTTQESHAL

SEQ ID NO: 88 - protein, 412 aa, unidentified
MWHVELKRTARVKLAIPDDRRDDLKRTMLTFREVAQRFADRGWERDEDGYVITSRTRLQSLVYKQVREDTGLHSDLCIGAVNLAADSLRSAVERMKAGKNVGKPTFTVPTATYNTGAVSYFTDGDGTGYCTLAAYGGRVRAEFVYPPDEDCPQRQYLGGDEWEPKGATLHYERDDGEYYLHVTVERDEPETELGEAENGTVLGVDLGVENIAVTSAGAFYSGGLFNHRRDEYERIRGSLQQTGTESAHRTIEKMGDRERRWNTDVLHRISKAIVQEAITHDCSHIAFEDLTDIRDRMPGAKKFHGWAFRQLYEYVEYKAAEFGIATTQVDPAYTSQRCSKCGTTLRENRTSQAAFCCQKCGYEVHADYNAAKNVATKLLRSGQKSPAGGATNQLALKSGTLNGNGDFTPASS

SEQ ID NO: 89 - protein, 437 aa, unidentified
MPTITLRLELYNPTKVKQDMYERMTEVNTAFANWLLNHPELNQATSKIFKEFSPQRFPSAVVNQTIREVKSQKKNQKTKKFQTLWCCFNNQNVKVEKKGSFYTVSFPTLEKRIGVPVVTRPYQEAWLNRLLDGTAKQGAAKLYKKRKKWYLAIAITFDVKPRHETKVMGVDVGLRYLAVASVGTKSLFFKGSQCAFIRRRYAALRRTLGKAKKLQMIRKIGRKESRWMKEQNHKISRQIVNVALANGVGVIRMEALTGIRKRAKSAKEAGRSLHAWAFHQLQTMIAYKAEMAGIRVEWVDPTYTSQTCKCGHREKANRNGIRFRCQRCGYTLHADLNGAINIAKAISGFAAEPSALVTGAPPIGVHRNPMGRGDDTPLKLLRCPNQKWMRTQTTQESHAFRRAECQIISSQTRVPRPDGSAGSTMSRRGRRWRISCT

SEQ ID NO: 90 - protein, 414 aa, unidentified
MKASRFLILNDYEKNGILSSPQGGESMPTITLRLELYNPTKVKQDMYKRMTEVNTAFANWLLNHPELNQATSKLFKEFSSQRFPSAVVNQTIREVKSQKKNQKTKKFRTLWCCFNNQNVKVEKKGEFYTVSFPTLEKRIGVPVVTRSYQEAWLNRLINGTAKQGAAKLYKKRKKWYFAIAITVEVQQREETKVMGIDLGLRYIAVASVGTKSLFFKGSQCAFIRRRYAALRRTLGKAKKLHMIRKIGRKESRWMKDRNHKISRQIVRFALANGVGVIRMEELTGIRKRETSAKEAGRSLHSWSFHQLQTMIAYKAEMAGIRVEWVKPTYTSQTCRCGHREKANRNGIHFQCKKCGYTIHADLNGAINIAKAISGFAAEPSALVTGAPPIGVHLNPMGRGDDTPLNLGVVRTRNG

SEQ ID NO: 91 - protein, 557 aa, unidentified
MANKNVNDSKIKTHILTKKVQLIVDTDRFDTDEEKKAEVDRVYKYLRDSMRCQSREMNQYYMHLWMMSVARNLNDDRYSMKKYMNNIIDAVHPYLDKNNKEFNNKQKKITRDFEKKIKNLCEEYQAETEILNNDTLRELNKCFNRKKDGAYDTNFVNEMPEGLGIVMGRTVEQDFQNDCKAGLLSGIRNPRSYKINYPLIIPKSFVAYGVGSGKAMQGRGIIFPDMEYSDFHNMLFSTKNPNITYNFVHDIDFKLVFGSMKRSHELRVIFDRIKMGEYSICGSTIEINNKKKIMLNLSYEAPIYEKPNLDENTVVGVDLGMAIPAVCSLNNDDRTYKYIGDSHELEFIKKGIQAQRRSYQRNAVYNKGGHGRNRKLENLDRLKKRERNTTRTHNQRYAKQIVDFALANNAKYINLEKLKGFSNNEKNKLVLRNWCYYELQQYIEIDASKYGIKVRYIYPMNTSRTCSVCGTLTTDEDVKNGVGRVSQDEFICKDPNCPSHTLYTRGPKGHKVPYFNADRNASRNIAMSEDFVKKNAKDNAFKKIDDLYEINNDIDAA

SEQ ID NO: 92 - protein, 498 aa, unidentified
MQLNTITLTVKLQIRPLGDKEAQNEVWRNLRNINRDVFKAANLLMSHLHFVEAFEHQFMQTDRILEKEMLDRENKKKNKKLNAQQIETIAQEIAEIKQKRKELKIEVAEKVKIFYNGKNESFAYNFIRHEYPQIPSYSVSILVKTVQNKFKEEWKEVKKGEKSISNFKKTIPIPIDDKQIANTDKGKKLCYIKKEGEKFVWELNSLEAKFEIVTGKYKQSKYMGEDKNKRTTLERVMNFEYKVADSSLKIEENKIFLLLVCKIPQQQATSLPNVSIGVDLGIRTPACIACNDGLRVISLGSKQDFLAIRQRFYEHRKRLQKSLAMTKGGKGREKKLKALEKLRKAERNWVRTYNHTLSKKIIHFAIQCRASRIQIELLEGFGRNENKVDEKGNYLLGKWSYYELQNMIKQKAEQYKLVVTTIDPYHTSKTCHVCGELGYRKGADFFCQNERCKEYQKRQNADHNAAFNIAKSTTFVAKKEDCTYFKLEQEKKSKKEEE

SEQ ID NO: 93 - protein, 399 aa, unidentified
MRRTVKIKLSLNQNQQKLLQETIKQFKQACQRVVDYGWNRNGLKTYQKNKLHKATYSEIREKTDLPANLVIRARDRASETIKACVKKIKNGEKASKPTFKSDSIVYDKRTLTVWLEEERCSIATTNGRIKADFVLPNEYNDYYDKYLNNGWKITQSTIEKHSYEDDEPFYLHLGLEKEIQKNQSVNPTIMGIDLGIENLAVTSTGRFYSGTELFSRRERYEEVRGKLQAKGTRSAHLTIKQMSGRENRFACDTLHRISKKIVEEAISKDVDVIAMEELEKIRQKISNNKKFQTWAFKKLQEYIEYKANERGIEVKFVDPKYTSQRCSRCGTTLKQNRINQHFECKDCGYRVDSDYNAAKNIGFKTILDGQMSQSRMGNGQLALKSGVLKPNGNYYSYPN

SEQ ID NO: 94 - protein, 409 aa, unidentified
MIVTRKIQIRPINKEHYKILEDYLRTCRLIANKSQTLFYVYWQEIMANNLKGKNIDAYFKERYQHSFKQTIYHILRPKHLEIPSRITDDTLNIAYKDFCNDLKNGLLKGERSLRTYNNGWIPVRSQTTKITKQNNKYILEWLKGIKLEVYFGRDKSGNQVVVDRILNGQSKFCDSKFIKKEKKWFLLLCVNEPEKENNLFDDISIGVDCGINIPAVCAVNKGYGRAYIGSSVLLTRFRMQKDQRQRQKNYISSSGMHGRQKVDRAIFKAQDFEHRYMHTFHHKISKEVIKFALKNRASKIIMEDLKRFGQNQEESEEKKKIRRFWGYQQLQSMIEYKAKLENIAVKYIPPAYTSQTCSQCGYTDANNRKQSKFVCLNPKKKCGFEANADYNAALNIAKGGIKKVNNKDV

SEQ ID NO: 95 - protein, 460 aa, unidentified
MPTITLRLELHNPTKVKQGMYERMTEVNTAFANWLLXLELHNPTKVKQGMYERMTEVNTAFANWLLNHPELNQATSKIFKEFSSQRFPSAVVNQTIREVKAQKKKQKAKKFRTFWCCFNNQNVKVEKKGVFYTVSFPTLEKRIGVPVVTRSYQEAWLNRLLNGTVKQGAAKLYKKRKKWYLAVAITVEVQQREETKVMGVDLGLRYIAVASVGTKSLXAKLYKKRKKWYLAVAITVEVQQREETKVMGVDLGLRYIAVASVGTKSLFFKGNQCAFVRRRYAALRRRLGKAKKLHMIRKIGRKESRWMKDQNHKISRQIVAKKLHMIRKIGRKESRWMKDQNHKISRQIVRFAVANGVGVIRMEALTGIRKRATSAKEAGRSLHAWAFHQLQTMIAYKAEMAGIRVEWVNPTYTSQTCKCGHREKANRNGIRFRCQRCGYTLHADLNGAINIAKAISGFAS

SEQ ID NO: 96 - protein, 496 aa, unidentified
MILTRKIKLVIVSENREEGYNLIRTEIREQHKALNLAYNHLYFEHNAIQKLKQNDEDYKQKRNKLQELINKKYEEHQKAKNLEKKEALREAYNNKKQELYNFEKEYNEKARQTYQQVVGFTQQTRVRNLINRECNLMSDTKDGITSKVTQDYKNDCKAGLLIGKRSLRNYKKDNPLLVRGRSLKFYKEDGDYFIKWNKGTIFKCILHIRKKNVVELQSVLENVLLGAYKVCDSSIGFNNKDMILNLSLNIPDKETQDYIPGRVVGVDLGLKIPAYLSLSDKVYVRKGIGSIDDFLRVRTQMQKRRRRLQKSLAAVKGGKGREKKLKALDHLKGKEANFAKTYNHFLSTQIVTFAVKNQAGQINMEFLEFDKMKNKSLLRNWSYYQLQIMVEYKAKREGIIIKYVDAYLTSQTCSKCDHYEDGQREKQENFMCKNCGLEVNADYNASQNIAKSTSYISDSTESEYHKKKQQVLKEILGENDIMNEQLSLFNNCDDIA

SEQ ID NO: 97 - protein, 471 aa, unidentified
VITARKVKLTITENREDGYNFIHNELREQNQALNMAMNHLYFNYVAREKIKLADETHKIKLAEDQGYLDQKYTELKEVKTDKKKQNIRKSIQAAKKRLETLRKAENKQVAEKFKEIIAASEKTNLRDFITDNFNLTSDTKDRLTQKVSADFKNDIVDVLRGERTLRRYKKGNPLYIRGRNLTFYIKDEEYYIKWMKSIVFKCVLGVKKQNSLELQKTLDKVIEGKYKVCDSSIEFKQNSLILNLTLNIPVCNSFDKVEGRVVGVDLGMKIPAYVTLNDSDYIRRAIGSIDDFLKVRTQMQSRRRNLQRALKSTKGGKGREKKLKALNQFEVKEKNFAKTYNNFISSNIVKFASDNKAKQINMEFLSLSETQNKSVLRNWSYYQLQQMIEYKANRVGIKVKYVDPYHTSQICSKCGHYEEGQREKQEVFICKNPECKNFNIEVNADYNASRNIAKSNKYITKKEESEYYKIN

SEQ ID NO: 98 - protein, 422 aa, unidentified
MMTKSVKIKLGTPIDSNWNTVRKILADLRYNSSKMLNFSIQQCYQWMIYRNEYREKYGKYPRAKDIYGYSHRNHIYRQAAEIFPIFNRGNISQTVGMATSRWSSDQKDVMSLRKSIPSYRLQAPIYIANQSYTISRTENGLVVDCSLVSKKYAKEETNDKTRYRISLVVKDNSTETILDRIVSGEYSQGYGQIVKDRRKEKWYLLVAYRFEPKKTKETGRILGIDLGIVYPLYMALNDSHHRYRIDGGEIEHFRRNIEKRKNQLLDQGKYCGDGRRGHGIRTRIAPIEFAREKIKNFRNTTNHKYSKFVVDIAQKHDVETIQLEKLDGISEDSTFLKNWSYYDLQQKIQYKAQEKGIKVAYIDPRYTSQRCSRCGNISRENRQDQQRFRCTKCGFSANADYNAAKNISTQNIEKIIEEELKK

SEQ ID NO: 99 - protein, 480 aa, unidentified
MTTKVMRYQLIEMVDNEKRFMYKMLDDLRYEVFKISNRAIQMFWDIDNTSYAFKQKFQENLDLKELTGVKSLAYISRALKEEYQKLNNTSVEQVSRKVEKEWKKNKSNMITADTSMIRYKRKNANIKLKNTQFKIEPLDNNFYRISARLLSKSYAKDLHENGFEFAQKFKEKGKKKEKTIKEFIKKNDDNMWVHFKIKAHDGSQKSIIERVVNKEYKVGGSDIIYCDRKRKYYLNLSYTFEAEQAKVDENKILGIDVGVNTPATLAISDDKWYKEFIGDKQEIENYRNQVESRRRRLQKNAALYSGEGSTGHGRKTRLKSVDKIRDKIARFKDYKNHIWSRAIVNEAIKHGCGTIQMEDLTGIAANTNEKFLKTWSYFDLQTKIQYKAEEVGIKVVKVKPAHTSARCNNCGHIHSKENKDKWRPKEFHHEKFICQNCNHTAHADLNAAKNIAMKDIEKIIKDQLESQEKYYKNQMKYILD

SEQ ID NO: 100 - protein, 512 aa, unidentified
MITTRKFKLAIVSDNRNEAYNFIRSEIRNQNKALNVAYNHLYFEHIATEKLKHSDEEYQQHLTKYQEVASNKYQDYLKVKEKAKASKDDEKLQKRVDKAREGYNKAQEKVYKIEKEFNKKSKETYQKVVGLSKQTRIGKLVKSQFTLHYDTEDRITSTVISHFNNDMKTGLLRGDRSLRTYKNTHPLLVRARSMKFYEENGDYYIKWIKGIVFKIIISAGSKQKANIGELKSVLINILDGHYKVCDSSISLNRDLILNLSLNIPVSKENVFVPGRVVGVDLGLKIPAYVSVNDTPYIKRGIGNIDDFLKVRTQLQSQRKRLQKALKSTSGGKGRSKKLKGLDRLKAKEKNFVNTYNHFLSKNIIQFAVKNNASVIHMEELHFDKLKHKSLLRNWSYYQLQTMIEYKAEREGIEVKYVDASYTSQTCSKCGHCEEGQRVLQNAFICKNKECKGYGHKVNADFNASQNIAKSTDIIRGTEIAKTNDTAKNTKSIKGNQQSDDEVERKQLELELN

SEQ ID NO: 101 - protein, 398 aa, unidentified
MSDTSPYKHQTKNIGLLVHPEVSKATSKIFKEFSHGKFPSAVVNQTIREVKSKKKKQNAKSFKKLWCCFNNQNLKMEKVGDFYTVSFPTLEKRIGVPVVARPYQQAWLERILNGTVKQGASELYRKKKEWYIAIPITFEVEQRETKVMGVDLGLRYIAVASVGTKSLFFKGNQVAFVRRLFAARRRKLGKLKKLSAIKKSKDKESRWMKDQNHKISRQIVDFALTNGVGIIRMEDLTEINRAKSKKEAGRNLHSWAFYQLQKMIKYKAEMVGICFELVKPDYTSQTCKCGHREKANRIGIQFRCKKCGYTCHADLNGAINIAKAHSGLVAPSVLVTGTPPMWVHSNHMGRGDDTPLNLGVVQNGNGLRTLTTQESHGFSRVECQYGRIICWTWTKTVY

SEQ ID NO: 102 - protein, 481 aa, unidentified
VITVRKIKLTIMGDEETRNRQYKWIKDEQYNQYKALNIGMSYLATHLFLKMSESGLEQKTEKNIKTIEKQISKIENNITKEESKKKVNEEKLNNLINELDTLNASLSKLKEELEEISNNRSNVDDTFKRMYVDDLYNALSKVPFQHSDMKSLVSRKVKLDFNTDMKDLMSGNRSVRNYKRNHPLLVRGRDLRFRYDGSNIKIKWIQGIEFKAILGKISKTIELRHILNKVIDGEYKVCDSSLEFNKNNHLILNLAIDFPYTNKIEFIEGRVVGVDLGIAVPAYVALNDIAYVKKSIGDIDDFLRVKTQMKKRRNLQINLTSVKGGKGRSKKLKALDRLSEKESNFVRTYNHFLSKSIVKFAIDNKAGQINLELLSENALSDKIIKNWSYYQLQQFIKYKAERYGIKVKYVDPYRTSQTCSVCGHYEEGQRSKQDIFTCKNEKCKMFEKEVNADYNAARNIAISTKYIDDIKESEYYYKKSF

SEQ ID NO: 103 - protein, 428 aa, unidentified
VKLVKTMRYQIIKPLSCDWDTLGTVLRELQRDTHSVLNKTIQLCWEWQGYSSEYKAANGTYPTPKDTLGRSLEGYVYDRLKVQFPKMYTPNLSQTIQRAMLKWSADSKAIFKGEVSIPSYKKDVPLDLRKDSIHIERRGHDYILSLGLVSRAYKKELGLPECQIEVLIGTPDKTQRVILARLLTGEYTVSGSQIVWDKRNRKWFVNLAYHFEARPEQLDKTKILGVDLGVVFPVYMAVADGHFRAGIPGGEIEEFRRRVEARRRQLLRQGKYCGDGRIGHGRATRTRPLDKIADKIARFRDTINHKYSRYVVETARKLGCGVIQMEDLTGIREENLFLANWPYHDLQRKIEYKAREYGIEVRYVRPQYTSQRCSDCGYIHPDNRPEQAKFRCLACGFETNADYNAARNIATEGIEELIAAALNKASVV

SEQ ID NO: 104 - protein, 450 aa, unidentified
VEGEKEKYVVQSYGIKLIKPVGVDEGDWDFAGKVLRDLDYICFRVKNKAATRTYMNVIEKLEYENVHGKGTYDKYFKTRYGKTFSAYNMEQAKEDKELNNGSFLREHFDSMAREGEKVVKNNMKKILNGSASNITFKRNQPIPIRSRMIFITKESGKYYAELTLLSPEKAKELDRKGRKGTRIKFLLSSKGQEKVILDRLTSGEYDLRDSHIHVKKRGSKLNNYLIVAYRLKVKDDKDLIPNKILGVDLGISKAAYMAVSDSPVSEYINGGEIEQFRNGIEARRNSMRNQLKYHSSNRSGHGRSTKLIPLEKLRAKVNNFKELTNHRYAKFIVDTALKNRCTIIQMEDLSGISKTDTFLKRWSYSNLQEKIENKAKTKGIEVKKVSPKFTSQRCNKCGYIDKESRKSQEKFECVNCGHKTNADLNAARNLSMLDIEKVIKAQCKAQKIKH

SEQ ID NO: 105 - protein, 462 aa, unidentified
VPIKALRVQIIKPFNTDYDSQPITWDELGRTLRDLRYAASKMANYVIQQNYMWEFFRQQYKQEHGSYPSVSEHKDKLYCYPRLTAMFPLAAGQMVNQIERHAKTVWSARKSEVLKLHQSVPSFKLNFPIIVHHDSYRISEVPEDGKSSTHVFLLQANLLSREAATRTRYSFLINAGEKSKQTIVERIISGEYRQGALQIVGDRKNKWYCIIPYEFKTEENNTLDPQCIMGIDLGISKAVYWAIIGSHKRGWIDGHEIEEFRRRVQARRKSIQEQGKYCGDGRIGHGRKRRLLPIEVLENRESNFKNTTNHRYSRFIIEAAIKNQCGVIQMEDLSGINERSTFLRNWTYYDLQMKIKAKAEEVGIEVRIVNPQYTSQRCSQCGHIDRDNRSNQATFVCTHCGYGGLYHCFACGKSQVEAGVCHLCGGETKIMKINADYNAARNLAICGIDQIIVQTLEGEGVR

SEQ ID NO: 106 - protein, 438 aa, unidentified
MQKVLKVQLICEHFDQEGNAVDYKTICKLLWELQKQTREIKNKSIQYCWEYSNFSSDYYKEHHEYPKEKDILSYTLGGYVNDKLKAGNDLYSANCSTTIRTACAEFKNAKSDFLRGDKSIISYKANQPLDLHNKSIRLEYQNGTFYFWLKLLNKSAVKANGFRNTEIRFKALVKDNSTRTILERCAASVYDIAASKLLYDRKKKCWYLNLVYAFEPQLAKALDPEKILGVDLGIHYPICASVFGDLKRFTIDGGEIEAFRRRVEARKISMLKQGKNCGEGRIGHGIRARNKPVYAISDKIARFRDTMNHKYSRALIDYAVKNGCGVIQMEKLTGVTADANRFMKNWTYFDLQTKIEYKAKEAGIQIIFIDPHYTSQRCSKCGYIDRENRPVQSRFSCQKCGFTENADYNASQNISIRNIEKLIEAQLQTKCESQADNS

SEQ ID NO: 107 - protein, 442 aa, unidentified
LATKCIKLAGEYAKENSLEKDKFFKELRDIQYKTWLACNRAITYYYSNDMQNFIQKDVGIPKEDDKLLYGKAFKSWVANRISEILGDGISSYATDCISQFVSNRYKNDKKAGLLKGNVALSQFKRDIPVMLRERAYSIIDTPKGLGIEISFFSKTKQQELGIKRILFTFPKIDGSSKSILTRIMDKTYKQGSIQITYNKRKKKWMFAISYTFENKLEKVLNDNLVMGIDLGITKVATMSIYDIEKHQYKNMCFKEQTIDGTELIHYRQKIEARRKSLSISSKWASDNATGHGYKRRMKKANNIGDKYNRFKDTYNHKVSRYIVDLAYKHGVKTIQMEDLSGFSEHQSESLLKNWSYYDLQNKIKYKAEEKGINTIFINPQYTSKRCSKCGNIHEENRDCKNNQAKFECIICGHKENADINASKNIAIPYIDKIIKEYIKDTI

SEQ ID NO: 108 - protein, 413 aa, unidentified
MKITKQTKIRFNFQSPNAETNRAVYEKLNGCIYLTWKAFNRAYNEFIFQHINKQKKGENYTAEENKKFRNSGYRSILDIDIHSSIKASISQKAYKQFLNDTKKGGVLSGSRTWSSYKLPSPIPFQIRMLSIFKDTDGYFMRIPYFSKDFPFNLMIERNEQKVIVDRILSKEYELNDSSIQKDKIHNKWYINLCYSFDKEPDSSIDKSIVVGVDLGIAIPVMCAINSSEYIRGSFGNRQEIDNFRARIKSKRWQILKQNNSFYDLRTGHGKSGKLKPLIPLEDRIKKFMNTYNHKLSHAIVAFALQNKAGIINFENLENLSEVKQKNMYLRDWNNADIITKTEYKAKEQGIEVHFINPAYTSQRCSKCGHIEKENRESQSEFKCTKCGYEANADFNAARNISQMNDKSSLKIDT

SEQ ID NO: 109 - protein, 417 aa, unidentified
MQRSVTLKIIRPEDEKISWEELGYLLRGLSFKVCRMCNFCMTHQLLHALKLETELLNPQGNLYCYPRLAEEYPDVPTGIICAAETRARKLFRRSAEAVLHSETSLPRFRKDSSIPVPVAGYKILQDADHNVYADVQLLSRQGAKTQKRPGRIRLVLADNWRDQSAKAALQQIAAGKVKRGVASLFRVKNDWYFQIPYVTEVVNTGEGFEPDLVMGVAFGLQNALVYAFNTSLKRGAISGEEVLAHQEKYAVRRKKIQEQYNWSGRKGHGREDALKPLRHLYETERNYRSLVNSRYAKWVVDIAVKNRCGMIHLDSANYVSSGKKILLSRWPLYDLKEKIRRKAEEKGIQVTECSIPNLRTRCSRCGKEQEPEGEKRTFVCKDCGYGKADKNRRGGFISVDYNAARNLAVYKSEEKEL

SEQ ID NO: 110 - protein, 431 aa, unidentified
MAKGTLSKVMKYELSYLDGCGDFQNMQKELWALQRQTREILNRTIQIAYHWDYTDREKFKKTGQHLDVKSETGYKRLDGYIYDELKEDVKNFASVNVNATIQKAWSKYKSSKTDVLRGDMSLPSYKSDQPLVLHGQSMKLSEGEDGVVMQATLFSNTYKKEQEYSNVRFAVRLHDTTQRTIMKNILSGDYGLGQSQIVYKRPKWFLYLTYNFSPKQHEADPDKILGVDLGETIAIYASSIGEYGGLRIEGGEVRAFAKQLEARKRALQKQATYCGEGRVGHGTKTRVADVYKAEDKIANFRNTVNHRYSKKLIDYAIQHQYGTIQMEDLTGVKKDTGFPKFLQHWTYYDLQQKIEAKAKEHGIRIIKVNPAFTSQRCSKCGNIDSGNRPSQAVFCCTKCGFKANADFNASQNISIPGIDKIIKESYGANME

SEQ ID NO: 111 - protein, 436 aa, unidentified
MNKVVRIYLISEHTDKKGDPVNYQDINKLLWELQKQTRTIKNKTIQYCWEYQNFSSDYYKEHHAYPSEKEILSYTLDGYVNDKLKNSSDLYSVNRSSTIRNAIKEFKNAKADMIKGVKSVISYKSDQPLSLHNQSVRIEYIGGQYFANIKLVNSPYAKEHDFASTMIRFKFWIRDKSAETIIQRCLSNEYKISESEMFYDRKKKQWYINLCYSFSASKNDSLDIHKILGVDLGIAYPMCASVYGDYARFTIHGGEIEKFRRTVEARKLSMLKQGKNCGEGRKGHGIKCRNKPAYNISDKIARFRDTINHKYSKALIDYALKNNCGVIQMEELTGITADADRFLKNWTYYDLQTKIKYKAEENGIKFKLIKPKYTSQRCSKCGYIDKENRKTQAHFLCLKCGFECNADYNASQNISIENIDMIIEQELKSDANIGCT

SEQ ID NO: 112 - protein, 440 aa, unidentified
MTKVVRVYLIIEQKDKNGDIVEYTKINKLLWDLQKQTRIIKNKAIQYYWEYKNFSRDYCEKNGNYPSVEEILTYKTVDGYINNRLKIDNDLCAINRSSTIKHAIAEYKNAESDIKDGTRSIINYKSDQPLDLHNTSIHIERVKEEYYLFANMVSREYAKANNFANARICFKLMLKNNKSAQTIIDRCLNGDYKISESKIIYDRKKKQWCINLAYSFSPSNVQQLDYNKILGVDLGITYPLCASVYGEYDRLAIHKGEIENFRNKVEARRYSMLRQGKNCGDGRIGHGIKCRNKPAYNIGDKIARFRDTTNHKYSRALIEYAIKNNCGTIQMENLAGITDKAERFLKNWTYYDLQTKIQYKAKECGIKIQIINPQYTSQRCSRCGYIHSDNRKTQENFLCLKCGFAANADYNASQNISIKDIDKIIKKNWKICEPDIYRIT

SEQ ID NO: 113 - protein, 468 aa, unidentified
MPTITRKIELTLCTEGLSEEQRKEQWGLLYHINDNLYKAANNISSKLYLDDHVSSMVRMKHAEYLSLLKELARAEKQKTPDADAIAELRKKVAAAETEMTDQEHAICKYATEMSTETLAYKFATEIETHVFGQILTCLKQAAQSNFNSDAKDVKRGERAIRNYKKGMPIPFPWNRSLKIEADGGNFYLRWFNGLRFLLNFGKDRSNNRLIVKRCMKMDADYEGEYKLCNSSIQIAKREGKTKLFLLLVVSIPQEHVELNKKIVVGVDLGINVPAYVATNITEERKAIGDREHFLNSRMAFQRRYKSLQRLKGTAGGKGRTKKLEPLERLRKAEHNWVHTQNHLFSREVVDFAVKAHAATIHMEDLSGFGKDNDGNADEKKEFVLRNWSYYELQNMIAYKAAKYGIKVEKIRPAYTSKTCSWCGQQGFREGVTFICENPACKQCGEKVHADYNAARNIANSKAIIKKNE

SEQ ID NO: 114 - protein, 468 aa, unidentified
MPTITRKIELTLCTEGLSEEQRKEQWGLLYHINDNLYKAANNISSKLYLDDHVSSMVRMKHAEYLSLLKELARAEKQKTPDADAIAELRKNVAAAEKEMTDQERAICKYATEMSTQSLSYRFATELETNIFAKILDCLKQGVFATFNSDARDVKRGERAIRNYKKGMPIPFAWNDSLRIEKDNKDFYLRWYNGLRFLFNFGKDRSNNRLIVERCLKMDADYDGEYKLCNSSIQIAKREGKVKLFLLLVVSIPQEHVELNKKIVVGVDLGINVPAYVATNITEERKAIGDREHFLNTRMAFQRRYKSLQRLKGTAGGKGRTKKLEPLERLRKAEHNWVHTQNHLFSREVVDFAVKTHAATIHMEDLSGFGKDNDGNADERKEFVLRNWSYYELQNMIAYKAAKYGIKVEKIRPAYTSKTCSCCGQQGFREGVTFICENPECKQYGEKVHADYNAARNIANSKEIIKKNE

SEQ ID NO: 115 - protein, 445 aa, unidentified
MPTKCIKVALEFIKKDNNISEKQINKELKDIQFKTHLACNRAMTYMYSNDSETIIQRDIGIPKEDDKFLYGKSFGSWIENRMNEIMDGVLSNNVAQTRAFVINTYNQDKKNGLFKGNVTLSQFKRDMPIILHNKAFKIIETSKGLGVEIGLFNLKKQKELGIKRIVFLTPRLGESEKSIFKRLMDKSYKLGTAQISYNQRKRKWMIAISYTFNKEKDTIYLDNNKVMGIDLGIVNVVAMSIYDNAKEQYIKMSWKDRLISGTELISYRQKLESRRKNLAIASKWASSNRCGHGYKNRMRAVNKQGDKFNRFKDTFNHKISRYIVNMALKYHAGIIQMEDLSGFSNEQSESLLKNWSYYDLQEKIIYKAKENGIEVILIDPKYTSKRCSECGNIDDKNRDCKKDQEHFKCTACNYTDNADINASKNIAILKIDSIIENYLKNKNKG

SEQ ID NO: 116 - protein, 439 aa, unidentified
MIKIVKTMKYEIKYDKELYNLLSDIQHAVWLIKNRATTAAYDWQQFSFAYNERFGEYPKEKDVIGKTLAPDVYGFLKEIGSFVSSSIVDSAVQEAITKFKNDKVKILKGEQSIQIYRRNGSFPIRASQLKGLTKLDNKTYNAKLSLLSNEGAKERDCKGQFLVTLVTGNGAYEILDRVINGEYKMCDSRIYKRKNKFYLLLTYKFEKETDKVLDENRIMGVDIGVAVPAVLAINEDKFYRQYVGDAKEVSDFVAQINDRKKRLQRSRKWAGEGSRGSGRKKLMKPVDAISNKIHNYRETKNHTWSRFIVNEALKNECGTIQIEDLSGISKDNAFLKEWTFYSLQQKIIDKAKEHGIQVVKVKPNYTSQRCNKCGFIHKDVNKEIWRPTQSSFKCLNCGHETNADLNAARNIAMKDIEKIIVDQLNVQEQHQKHAEKYLV

SEQ ID NO: 117 - protein, 469 aa, unidentified
MGAKIVKLQLIYKSDEVMPYKDYCKELFALMREMSIIKNKIATYLYLDKRFSVALPIEKDTKVKSASGLASAFASELLVNYKTGEVEVLKNHSSNRSAAGQDVQAKFKKFLKESMGAYAVNPPSFKDNGTLCLHDRCIQIYYDEDNKDYGAKLFLLSHSYAKELGIKSSNGFDYKLLVGDDSSRAIIERIIAGEYKISASNLKWDKRKKKWYLLLCYSFTEKRTTSYDPKEYENNVMGINFGVICPMYMSFNHCKNRYNIEANEIEAFRKQVEARRIALRRQRKYCGDGSIGHGKNKRNSPAMKIDDKIARFRDTCNHKYARYAVDMAIKHQCGIIVIEDLTSIAEKEERLFLKTWSYYDLQNKIEYKAKEAGIKVIKINPQYVSRRCSKCGYISFEKEENIKAPDYRKFHCVECGFQSHVDYNASQNNATIGIEAIIAEQIKEHNSENVKNQPAAKAAKSKSKIKETA

SEQ ID NO: 118 - protein, 476 aa, unidentified
MITSRKIKLAIVSDNKDTAYSFIREETRNQNRALNVAYTHLYFEYVAQEKLKQSDKEYQQHLEKYKNAAAKKYQEFLTIKEKSKSDENLQPKMDKVRETYNKAMEKVYKIEKDYSKKAREIYQQSVGLAKQTRLGKLIKSEFDLHYDTVDRIGSNAMSDFSNDRKSGILSGERSLRNYKKTNPLMVRARSMKLYEEDNNFYIKWINDIVFKIIISAGSKQRMNIAELKSVFIKLLSGQCKMCDSSISLDKGLILNLSIDMPITKENVFIPNRVLGVDLGLKIPAYVSLNDTHYIKGAIGNIDDFLKVRTGLQSQRRRLQKSLQSTGGGKGRRKKLQALERLKTKEKNFVNTYNHFLSKNIVQFAVKNNAGAIHMEELKFDKMKNKSLLRNWSYYQLQTMVEYKAKSEGIEVYYVDASYTSQTCSKCGNLEEGQREARDTFVCKKCGYNVHADYNASQNIAKSTKAINKTIEITNIV

SEQ ID NO: 119 - protein, 437 aa, unidentified
MTKVTKVYLISEQIDKDGNKIDFKKISELLWNLQRQTRDIKNKCVQLCWEWLNFSSDYYKKSEEYPKEKDTLGYTLSGFVYDRIKNGSDLYSSNLSTSSRDTCTAFSNYKKEMLNGERSVLSFKANQPLDIHNKAIKLSYENGNFFVALKMLNRAGKEKYGINDDLRFRMQVRDKSVRTILERLMNDEYKVSASKLMYDKKKKLWKLNLCYSFDNHVISTLDPEKIMGVDLGVVYPIMASVNGDYARFSIKGGEIEAFRNRVEARRRSLLNQSRYCGDGRIGHGRKKRTEPAAQIADKIARFRDTTNHKYSRALIDYAIKNGCGTIQMEKLTGITSNAEHFLKEWSYFDLQTKIESKAKEAGIKVVYINPKFTSQRCNKCGYIHTDNRPVQARFCCQKCGYEENADYNASQNIGTKHIDVIIEETLKMQCEPEVPTE

SEQ ID NO: 120 - protein, 439 aa, unidentified
MIKVVKTMKYEIMYDKELYDLLSEIQYAIWLIKNRATPAVYDWQQFSFSYNERFGEYPKEKDVLGKTLAPDIYGFLKELGSFVSSQIIDTAVQEAIKKFKNDKMKILKGEQSIQIYRRNGSFPIRASQLKDLTKINNKTYEAKLSLLSNAGAKERDCKGQFLVKLVTGNGAYEILDRIINGEYKMSDSRIYKKKNKFYLLLTYKFEKETEKVLDENRIMGVDIGVAVPAVLAINEDKYYRQYVGDAKEVSDFVAQINDRKKRLQRSRKWAGEGSRGNGRKKLMKPVDAISNKIHNYRETKNHTWSRFIVNEALKNECGTIQIEDLSGITTDNAFLKDWTFFSLQQKITDKAKEHGIKVVKVKPNYTSQRCNKCGFIHKNVGKEIWRPTQSSFKCLNCNHVTNADLNAARNIAMKDIEKIIEVQLKAQEQNAKHEEKYLV

SEQ ID NO: 121 - protein, 431 aa, unidentified
MPKQISKVMKYELKYLGEEDFYEMQKMLWSLQEDTREILNKTIQIFFHWDYTNKESLETTGKALNLVEETGYKDISGYVYDKLKSRYPDMSRGNLSATIRAASKKYRSSKVDILKGTMSIPSYKKDQPIILRPDGIRLHEREMIYTVELSLFSGDFKKKKAWKSNVLFQIKACDKAQKAIMQRLLSREYKLGESKLVYKKKKWFIYITYSFKKTDAKLDKNKILGVDLGVTYAIYACSIGEYGSFSIKGEEALEYAKRLEARTISKQKQARYCGEGRIGHGIKTRLSTVYSTRNKLANHRDTLNHRYSKAVVDYAVKNRYGTIQMENLSCIKKNTGFPKRLQHWTYYDLQSKIEYKAAEQGIQVIKINPKFTSLRCSQCGCIHKDNRKTQESFQCVECGYKDNADHNAALNISIPQIDLIIKEEMTSAKEK

SEQ ID NO: 122 - protein, 488 aa, unidentified
MAEHTVITRKIEVHLHRSGDSEEAKELLREKYHMWDTINDNLYKAANLIISHCFFNDAYEDRLRIQSPRFKQIQDSLRNAKRNKLGESEIKELKSEREQLFDEFKKQRYTFLRGGVAEGPNPEQNSTYRVASDNFLDTIPSDILTCLNKNITTTYKSYRKEIEFGNRTIPNFKKGIPVPFPIKKDKKLRISKRDDGSIFIKFPGRLEWDLDFGRDRSNNREIVERVLNGMYDVGDSSIQETRSGKRFLLLVVKIPKVKNVSLDSNRVVGIDLGINTPLFAALNDNDYERVSIGSRDQFLNVRNRMNAQKREMQKNLRSSTTGGRGRRHKLQALERLEGKERNWVHLQNHIFSKGAIEFAIKNNAGVIQMERLTGFGRNANDEVENDRKFLLRNWSYFELQQLIEYKADAAGIEVRYIDPYHTSQTCSFCGHYEAGQRVDQAHFICKNPECEKGKGKKNDDGTYAGINADWNAARNIARSNKIVDRKKK

SEQ ID NO: 123 - protein, 424 aa, unidentified
LILTRKIQIIPLGEKEEIDRVYKYLRDGIFYQNKAMNQYMSALYIAAIKDISKEDRKELNRLYSRVSNSKKGSAYDKSIEFAKNMNLGYVVKQVKQDFANSCKNGLLCGKVSLPTYRKNNPLLVHVNFVRLRSTNYHQDNGMYHNYESHTDFLDHLYSKDLEVFIKFANNITFKMIFGNPHKSAYLRSEIQQIFEENYKVCGSSIQIDGKKIILNLSMDIPKQELELDENIVVGVDLGLAIPAMCGLNTNDYIRQSIGSKDDFLRIRTQLQSQRRRLQKSLASTSGGHGRQKKLKPLEKLKDRERNFVKTYNHYVSKNVVDFAVKNKAKYINVEDLSGFDSNQFILRNWSFYELQQFITYKAAKYGIEVRKINPYHTSQICSCCGHWEEGQRIDQAHFKCKSCGAELNADFNASRNIAMSTDFV

SEQ ID NO: 124 - protein, 468 aa, unidentified
MKTKKHTKVMRYEIIKPLDSTWEVFGQVLRQVQYETRHALNKSIQLSWEWQGFSAEYKRQSDHYPNLKEITNYTNLQGYAYNTLKHEFPSLYRGNFSQTVKRATDKWNSDRGEILRGERSIPNFKKDIPIDVVAAAFKEGKEQNLRIRKILSSEEYNNETGKRIQGYTVKVNLISDSYKKELGRNSTAFEMLIKVGDNTQKAIIERLIEGKYNIAASQILYQKGKGSGKKGKWFLNLSYSFEKEDVTLNPDLIMGIDMGIVHPIYIAFSDSYARYHINKGEINRFRNQIEKRKKELLHQGTYCGEGRKGHGIQARIKPIEVISDKIANFRKLCNHRYSKFVVDIAVKHGCGTIQMEDLQGISKDDVFYKNWSYFDLQEKIAYKAKESGIKVIKVNPRYTSQRCSQCGNIDSENRVNQADFLCTACGFKALADYNAARNISTRNIEKIIDKAVGKEIIDVVDLEYGNIS

SEQ ID NO: 125 - protein, 429 aa, unidentified
MVKVVKIYLISEQVDEQGKDVDYNTICGVLWDLQWETREIKNKTVQLCWEWSGFSSDYYKKYGEYPKEKNLLDYTMGGFVYDKLKSKYHLYTANLSTTSQNTCGIFRTYKVDFVKGNRSVLSFKADQPLDVHKKSISIDRIDDNYFVKLKLLNKSGIQKYGIRDDFHFRMLVKDNSTKTILERCVGGDYKAAASKIIYDKKKKMWCLNLSYEFDVNTAKDLNKNRILGIDIGIVYPVVASVNGELDRFVIQGGEIETFRRRVENRKKSLLKQTKYCGDGRIGHGRNKRTEPVDIISDQIARFRNTANHKYSRAVIDYAVRKQCGTIQMENLKGITDKSDRFLKNWSYYDLQQKIEYKAKEKGINVVFINPKYTSQRCSRCGYIDSANRPKLPNQSKFLCIKCGFTENADYNASQNIALYNIEKLIDAEA

SEQ ID NO: 126 - protein, 424 aa, unidentified
MPTRVMKYQIVKPMNCDWKLLNRHLLDLQQEAHQILNKTIQLCWEWQGFCSEYKAKYDIYPVDKDIFHLTLAGYVNRMLRLQFPKSNSNNLGTSRIKAINRWHTDLKDVKSGQKSIASFKADVPIDLHYLSIKLHKEKDCYYADLSLISNIYKKELGRESGKLLVLLKSGDEVSRDILRNCLDGIYKIRASDISHKKNKWFLNLHYNYEETNKSLDKDRILGIDMGIAYPVYMSVYNTQITSYIEGGEIERFHKQVEKRSAEFQRQGKYCGNGRIGHGVKTRLKPLSFATDKIANFRETTNHKYSKYIVDFAVKNNCGNIQMEDLTGIKNERIFLRNWTYFDLQKKIRYKAEENGINVLLIKPHYTSQRCSQCGDIDKKSRITQENFTCISCGYQTNADHNASINISTPNIENIINDFMSLKID

SEQ ID NO: 127 - protein, 439 aa, unidentified
MFMTKVTKVYLISEQIDKDGNKIDFKKISELLWNLQMQTRDIKNKCVQLCWEWLNFSSDYYKKSEEYPKEKDTLGYTLSGFVYDRIKNGSDLYSSNLSTSSRDTCTAFSNYKKEMLKGERSVLSFKANQPLDIHNKAIKLSYENGNFFVALKMLNRAGKEKYGIKDDLRFRMQVRDKSVRTILERLMNDEYKVSASKLMYDKKKKLWKLNLCYSFDNHVISTLDTEKIMGVDLGVVYPIMASVNGDYARFSIKGGEIEAFRSRVEARRRSLLNQSRYCGDGRIGHGRKKRTEPATQIADKIARFRDTTNHKYSRALIDYAIKNGCGTIQMEKLTGITSNAEHFLKEWSYFDLQTKIESKAKEAGIKVVYINPKFTSQRCNKCGYIHTDNRPVQARFCCQKCGYEENADYNASQNIGTKHIDVIIEETLKMQCEPETPTE

SEQ ID NO: 128 - protein, 425 aa, unidentified
MNKVMRYQIIKPIDIDWKTFGDILNKMRQEVRFTKNKTIALYNDWLTYCFQYKNEHNEYPKLINYCGYKVFSGYAYDKFKTDVVFSNTANYTTSVREACSAYDTHKTDILKGNCSIPSMGANQPIDLHNKSLSVDVNESGDYIATISLLSNRGKKEFGLKSGQIKVVLKAGDKSSKDILQRCVSKEYKICGSKIIYKGKKTFINLCYGFEPEASDLDKSKVMGIDLGVSVPAYMAFNFDKYKRDSIKDNRIMTTKWMMDQQLSIAKQSCKYLSDGNSGHGRKKKMICYDKYSNKSRNLSQTINHGWSKYIVDVAFRNGCGTIQMEDLSGVTSEKDKFLKNWTFYDLQQKIEYKAKEKGIDVVKINPRYTSQRCSECGCICKRNRPDQKTFKCISCGNSSNADFNAAKNIATIGIEDIIANTEVIE

SEQ ID NO: 129 - protein, 450 aa, unidentified
MSIAVKVMKYQIVCPVNVEWKVFETYLRTLSYQSRTIGNRTIQKIWEFDNLSLNHFKETGEYPSAQHLYGCTQKTISGYIYDQLKEEYQDINKANMSTTIQKTLKNWNSRKKEIWRGEMSIPSFRNNLPIDIHGNSIQIIKEKSGDYIASVSLFSSKFIKENDLPNGKILVKLSTRKQNSMKVILDKIVNSIYAKGACMLHKHKNKWYLSITYKATIKEEHKFDEELIMGIDMGKINVLYFAYNKGLVRGAISGEEIEAFRKKIEYRRISLLRQGKYCSENRIGKGRKKRIKPIDVLNDKVAKFRNATNHKYANYIVQQCLKYNCGTIQLEDLQGISKEQTFLKNWTYFDLQEKIKNQANQYGIKVVKIDPSYTSQRCSECGYIHKKNRQDQSTFECQQCSFKIHADYNAAKNISVYNIEKVIQKQLELQEKLNLTKYKEQYIEQMENIN

SEQ ID NO: 130 - protein, 453 aa, unidentified
MTYLSIAVKVMKYQIVCPVNVEWKVFETYLRTLSYQSRTIGNRTIQKIWEFDNLSLNHFKETGEYPSAQHLYGCTQKTISGYIYDQLKEEYQDINKANMSTTIQKTLKNWNSRKKEIWRGEMSIPSFRNNLPIDIHGNSIQIIKEKSGDYIASVSLFSSKFIKENDLPNGKILVKLSTRKQNSMKVILDKIVNSIYAKGACMLHKHKNKWYLSITYKATIKEEHKFDEELIMGIDMGKINVLYFAYNKGLVRGAISGEEIEAFRKKIEYRRISLLRQGKYCSENRIGKGRKKRIKPIDVLNDKVAKFRNATNHKYANYIVQQCLKYNCGTIQLEDLQGISKEQTFLKNWTYFDLQEKIKNQANQYGIKVVKIDPSYTSQRCSECGYIHKKNRQDQSTFECQQCSFKIHADYNAAKNISVYNIEKVIQKQLELQEKLNLTKYKEQYIEQMENIN

SEQ ID NO: 131 - protein, 436 aa, unidentified
MNKVVRLALICEHSDKDGNPVDYSDVYKLLWQLQAQTREIKNKTIQYCWEYSNFSSDYYKENHEYPKEKDVLHYALDGFVNDKFKVGNDLYSSNCSYMTRKVCAEFKKSKSDFLKGTRSIISYKSNQPLDLHNKSIRIEYKDNDFFAFLKLLKRPAFNRLGYKNSEIGFKVIVRDKSTRTILERCVDQIYGISASKLIYNKKKKQWFLNLVYAFEPDNANNLDPSKILGVDLGIHYPICASVYGDLQRFTIHGGEIEEFRRRVESRKLSLLKQGKNCGDGRIGHGGKTRNKPVYSIEDRIARFRDTVNHKYSRALIDYAVKKECGTIQMEDLSGITAESDRFLKNWSYYDLQSKIEYKAKEKGIKIVYIDPKYSSQRCSKCGHIDKENRKTQSSFVCLKCGFEENADYNASQNIGIKDIDKIIENDLSSKCETDVN

SEQ ID NO: 132 - protein, 433 aa, unidentified
MGKGVLAKVMKYELRYLDGCGDFSNMQEQVWALQRQTREILNRSIQIAFQWDCANSEHHRKTGEYLDLKTETGYKRLDGHIYNCLKGQYEDMATSNLNATIQKAWKKYNSSKKEILRGSMSIPSYKMNQPLTLDKNTVKLSEGERNPIVTLTLFSDKFKRAQGVSNVKFSMPLHDGTQRAIFANLMNGTYQLGECQLVYKRPKWFLFVTYKFPPVEHPLDPDKILGVDMGEACALYASTFGEHGYLKIDGGEITKYAKKMEARIRSMQKQAAHCGEGRIGHGTKTRVSVVYQAKDKVARFRDTINHRYSKALIDYALKNQCGTIQMEDLTGIKEDTGFPKFLRHWTYYDLQSKIEAKAAEHGIQVVKINPRHTSQRCSRCGHIDKANRTSQADFCCTKCGFSANADFNASQNISIRNIDKIIAKAIGANRKQT

SEQ ID NO: 133 - protein, 459 aa, unidentified
MRIKIIAKKKGINMNKIMKYQILKPTNISWEDFGNILYNLRSEVRKIKNRTIALYHEWTNYTLECHDRTGEWPKPKEVYNYGTMGGYIYDRLKGEVKYSNSVNFNSSVRDAMSKYDTHKKDILAGKASVPSMGDGQPIDIYNKNIVLHHLDNEKKDYAATLSLLNNGAKAELGLPSGRVDVILTIKNETQTAILDRCLSGEYRICGSQLIYEAAGKEKKGKKDKPKVWLYLCYGFEPEAPELDDSRIMGIDLGMKLPAVMAFNFNDKKYEVIDDNRILDRKIRLDKMLSMSKHQCQWRCDGNSGHGRKKKVGVYENYANKSHNMSMTINHQWSKYIVDTAVKNKCGVIQMEDLSGIKASRQNFLGNWTYYDLQQKITYKAEEKGIKVIKVDPQYTSQMCPICGYINKRNRATQADFECLECGHIANADYNAARNIATPDIANIIKNRLTQQKKEGKSID

SEQ ID NO: 134 - protein, 434 aa, unidentified
MNENMCALTKIMKYELRYLDGFPDFSAMQNAVWPLQRQTREILNRTIQEAYHWDYFSATKKKETGEYPDLLKETGYKRLDGYIYHVLAPDYPDFSSSGVNATIQKAWKKYKSSRADVWKGEMSLPSYKSDQPIVLHAKQIKLSGDNRAAAVTLSLFSNKFKKEHAISGNVQFAITLHDNTQRTIYQKLRNGEYKLSESQLVYDKKKWFLYLAYSFNPAEHALDPEKILGVDMGEKFALYASSFGEYGHFKIEGSEVTEYAKALERRRRSLQQQARYCGEGRIGHGTKTRVGAVYREEDRIANFRSTINHRYSKALIEYAVKNGYGTIQMENLTGIKENLQFPRRLQHWTYYDLQSKIEAKAKEHGIAVVKVNPKHTSQRCSRCGHIAAENRPKQEVFQCVKCGYACNADFNASQNISIKDIEKLIQETIGANPK

SEQ ID NO: 135 - protein, 449 aa, unidentified
MQQVVRFEIIKPIDNDWKVLGKVLRDLQYESRQVLNKTIQYNWEFSNCAIGFKEKFGINPKISDISKYKGSHALQNYIYNELKNVYTKISTSNLTCLIEKATSQWKTFQKDVYKGERNPPEYKKSNTPIIIHKQCINILKENGKYYADIALVSKKHLEEYKLNSCRFTLLLNTKNNGNKAILDMILNGELDYSQSQIIEKNKKWFLYLSYKVAEKVLPDDYNRIMGIDLGVNKAVVIAIHGTEIRDYIPGGEIIAYKNKMYNMVWHRQKQARYCGEGRIGHGRKTRLKNIYKIKEKIANFSDLTNHRYSKYIVELAKKHKCGVIQMEDLSGLSTNNKFLKKYPIYDLQQKIIYKAEREGIKVVKIKPNYTSQMCSNCHFISEENRPKDERGWEYFKCVNCGLEIDADLNAARNMANSQIENIIKEQLKIQGIKNKNNGTDEESRKAVNS

SEQ ID NO: 136 - protein, 422 aa, unidentified
MKYELTYLDGCGDFHNMQNELWALQRQTRELLNRTVQIAYHWDYKGSEHYRETGQPMDVYTETGYKRLDGYIYSCLKDDADSFSAANVNATIQKAWKKYKSSKSDIMQGKMSLPSYKRDQPLILYARNVKISNEQRNPAVQFTLFSKSYKEEKGYSDVQFVMHLNDATQKAIFQKIASKEYGLGECQLVYSKPKWFLLLTYNFTPEDRRLDPDRILGVDMGETFALYASSKDEYGSLKIEGGEVREFAKRMEARKRSMQRQSAHCGEGRIGHGTKTRVSDVYKAEDKIANFRSTVNHRYSKKLIEYAVQHQYGTIQMEDLSGIRESTGFPKFLRHWTFYDLQQKIEAKAREQGIRVVKVDPSYTSQRCSKCGNIDKANRPTQAQFCCSKCGYKTNADFNASQNLSIRGIDRIIKETLGANPK

SEQ ID NO: 137 - protein, 460 aa, unidentified
MSIKAIRLEIVKPYNESDAENVVTWNELGEALREVRYACSKAQNYVITERYLWERFKIDYKNQNGVYPDPKAFKERTNLYSQLTKMFPHVASNIINQTDRLATKKWSNEKKDVLSLKRSLTSFKLDVPIPVHYEGYKIFKVNDSEREKYIIRVTLLSKKSDKQMAYNLLLKVKDNSSKTILDRLISEELDSKIIQIVSTNKKKWFCIIPYDFTERNTEVVDGRIMGIDLGIAKAVYYAFNDSYKRGSIDGGEIERFRKSVRARRITIQNQGKYCGDGRIGHGVKRRLKPVEILREKEKNFRNLINHRYSRHLVEIAVKNKCAVIQMEDLTGITKDNAFLKDWPYYDLQQKIKEKAAEYGILFKTINPYKTSQRCSRCGYIDRENRPEQSVFVCQNCGYGSLYLCENCNKEQNHAGICDTCGGKTNLITVNADYNAAKNIATENIEEIIKKEMGKEYNPPK

SEQ ID NO: 138 - protein, 431 aa, unidentified
MSVKIASFSLIYKEDYNELNYDDLYNVLIQLQNESAKIANRAVQIYWENSNYCRQVKSMTGRFPTKDELLKHYGCSEQNYVYRLLTKEFYKNSTGNISTIIQFVGKRYKRYYPDYLSGKRSIESYKSSFPIYLCKNNIRVFKEDGKYYIKLGLISALYKSELSIHSGSVIFELGFGKSSSYKTLLDNIINQLFSLTSSKIIFLKKKIIIQLGYNTNSNIILDNSSCRVMGIDIGVAKPFVYAFNDITDFNFVDGNEIKNFQKQMLSRRQSLGRQTKNCADSKIGHGIHKRIEGIEKLGQKESNFRNRINHQYSRMIVDAAIKYKCTTIQIEDLSGISSENKFLKSWPYYDLQSKIEYKAKECGIDVVKINPKFTSQRCSKCGYISKENRKTQAGFKCKNCGFEENADLNAARNIAIPKIDSIIQESLRATT

SEQ ID NO: 139 - protein, 414 aa, unidentified
MVKTTKIYLCMDSKDTYKLLWKLQDNTRMLKNKAVQMLWEWNNFSQEYKKEHEEYPKPKDILNYTVGGYMYDKLKSESLLASSNLSSTLQLVEKQFKTNAKDFLRGDKSIICFKKDQPLDIHNKSIRLSHENGTFYADIVLMNKATANEHNGGGCALPFRLWIKDKSTRTIVERCYDGVYSVSGSKIKYDEKKKMWYINLSYGFDKYSTAELDKEQVLGVYLAQETPFTASVIGDHDRLQVSVNEIEHYRNAVESRRRSILKQSAVCGEGRIGHGYKKRVEPMEKLSHKVADTRDTINHKYSKAIVEYAVKKGCGTIRMEKLTGISETDRYLRNWPYFDLQTKIEYKAKERGIDIVYVDSADIQRCCRCGHVNEEETTGSRFKCTECGFEHDVAYNASQNLSAGGKDIKVNGRK

SEQ ID NO: 140 - protein, 469 aa, unidentified
MRLYSIKCNKEKKLNEVKRMESEKEKYVVQSYGIKLIKPVGVDEGDWDFAGKVLRDLDYACFRVKNKAATRTYMNVIEKLEYENIHGKGTYDKYFKTRYGKTFSAYNSECAREDKELNKGDLYREYFNLMAREGEKVVKYNMKKILNGNASNITFKRNQPVPITSRMIFITKESGKYYAELTLLSPEKAKELGRKGKNGTRIKFLLSSKGQEKVILDRLTSGEYDLRDSHIHVKKKGNKLTNYLIIAYRHKVKDDNDLIPNKVLGVDLGVSKAAYMAVSESPVSEYINGGEIEQFRNGVEARRNGMRNQLKYHSSNRSGHGRATKLKPLEKLREKVSNFRKLTNHRYAKFIVDTALKNNCSIIQMEELKGISKNDTFLKRWSYFDLQEKIENKAIAVGIEVKKVSPKFTSQRCNKCGYIDKESRKSQEKFECVNCGHKTNADLNAARNLSMLNVEKEIKAQCKAQKIKY

SEQ ID NO: 141 - protein, 421 aa, unidentified
MDKTLRDIQYVTCKASNKAMQMYYMWEYEKLEYKNKYGEYPNEKEMYGKTYRNVVEAEVKAIMNTINTSNTGQTNAFVMKKWNTDKKDIMNYRKSVASYKLNMPIYLKNSSYKILQGESGYEIDCAIFNKSQGLKHLTFTIDKLDGTKKATLNKLIESKNLSELINGAYKQGAMQIVKKKNKWCMLISFGFEAVKRELDTNRIVGVDVGIVNALTFQVWDNTTQKWDRLSWRDCLLDGKELIHYRQKIMARRIALLKNSKLSDENKGKAGHGRVKRIGPINTISDKVKAFRDTLNHKYSKYVIDFAIKNNCGCIQMEDLSGYSESVSETFLKNWSYFDLQSKIKYKAEENGLAINFIKPYHTSLRCSLCGNIQKENRDCRNNQSRFKCTVCGYEENADINAAKNISLPNIEQLIKEQLKIK

SEQ ID NO: 142 - protein, 450 aa, unidentified
MEGEKEKYVVQSYGIKIIKPVGVDEGDWDFAGKVLRDLDYICFRVKNKAATRTYMNVIEKLEYENVHGKGTYDKYFKTRYGKTFSAYNMEQAKEDKELNNGSFLREHFDSMAREGEKVVKNNMKKILNGSASNITFKRNQPIPIRSRMIFITKESGKYYAELTLLSPEKAKELDRKGRKGTRIKFLLSSKGQEKVILDRLTSGEYDLRDSHIHVKKKGNKLNNYLIVAYRLKVKDDKDLIPNKVLGVDLGISKAAYMAVSDSPVSEYINGGEIEQFRNGIEARRNSMRNQLKYHSSNRSGHGRSTKLIPLEKLRAKVNNFKELTNHRYAKFIVDTALKNRCTIIQMEDLSGISKTDTFLKRWSYSNLQEKIENKAKTKGIEVKKVSPKFTSQRCNKCGYIDKESRKSQEKFECVNCGHKTNADLNAARNLSMLDIEKVIKAQCKAQKIKH

SEQ ID NO: 143 - protein, 450 aa, unidentified
MEGEKEKYVVQSYGIKLIKPVGVDEGDWDFAGKVLRDLDYICFRVKNKAATRTYMNVIEKLEYENVHGKGTYDKYFKTRYGKTFSAYNMEQAKEDKELNNGSFLREHFDSMAREGEKVVKNNMKKILNGSASNITFKRNQPIPIRSRMIFITKESGKYYAELTLLSPEKAKELDRKGRKGTRIKFLLSSKGQEKVILDRLTSGEYDLRDSHIHVKKRGSKLNNYLIVAYRLKVKDDKDLIPNKILGVDLGISKAAYMAVSDSPVSEYINGGEIEQFRNGIEARRNSMRNQLKYHSSNRSGHGRSTKLIPLEKLRAKVNNFKELTNHRYAKFIVDTALKNRCTIIQMEDLSGISKTDTFLKRWSYSNLQEKIENKAKTKGIEVKKVSPKFTSQRCNKCGYIDKESRKSQEKFECVNCGHKTNADLNAARNLSMLDIEKVIKAQCKAQKIKH

SEQ ID NO: 144 - protein, 450 aa, unidentified
MEGEKEKYVVQSYGIKLIKPVGVDEGDWDFAGKVLRDLDYICFRVKNKAATRTYMNVIEKLEYENVHGKGTYDKYFKTRYGKTFSAYNMEQAKEDKELNNGSFLREHFDSMAREGEKVVKNNMKKILNGSASNITFKRNQPIPIRSRMIFITKESGKYYAELTLLSPEKAKELDRKGRKGTRIKFLLSSKGQEKVILDRLTSGEYDLRDSHIHVKKRGSKLNNYLIVAYRLKVKDDKDLIPNKVLGVDLGISKAAYMAVSDSPVSEYINGGEIEQFRNGIEARRNSMRNQLKYHSSNRSGHGRSTKLIPLEKLRAKVNNFKELTNHRYAKFIVDTALKNRCTIIQMEDLSGISKTDTFLKRWSYSNLQEKIENKAKTKGIEVKKVSPKFTSQRCNKCGYIDKESRKSQEKFECMNCGHKTNADLNAARNLSMLDIEKVIKAQCKAQKIKH

SEQ ID NO: 145 - protein, 450 aa, unidentified
MEGEKEKYVVQSYGIKLIKPVGVDEGDWDFAGKVLRDLDYICFRVKNKAATRTYMNVIEKLEYENVHGKGTYDKYFKTRYGKTFSAYNMEQAKEDKELNNGSFLREHFDSMAREGEKVVKNNMKKILNGSASNITFKRNQPIPIRSRMIFITKESGKYYAELTLLSPEKAKELDRKGRKGTRIKFLLSSKGQEKVILDRLTSGEYDLRDSHIHVKKRGSKLNNYLIVAYRLKVKDDKDLIPNKVLGVDLGISKAAYMAVSDSPVSEYINGGEIEQFRNGIEARRNSMRNQLKYHSSNRSGHGRSTKLIPLEKLRAKVNNFKELTNHRYAKFIVDTALKNRCTIIQMEDLSGISKTDTFLKRWSYSNLQEKIENKAKTKGIEVKKVSPKFTSQRCNKCGYIDKESRKSQEKFECVNCGHKTNADLNAARNLSMLDIEKVIKAQCKAQKIKH

SEQ ID NO: 146 - protein, 450 aa, unidentified
MEGEKEKYVVQSYGIKLIKPVGVDEGDWDFAGKVLRDLDYICFRVKNKAATRTYMNVIEKLEYENVHGKGTYDKYFKTRYGKTFSAYNMEQAKEDKELNNGSFLREHFDSMAREGEKVEKNNMKKILNGSASNITFKRNQPIPIRSRMIFITKESGKYYAELTLLSPEKAKELDRKGRKGTRIKFLLSSKGQEKVILDRLTSGEYDLRDSHIHVKKKGNKLNNYLIVAYRLKVKDDKDLIPNKVLGVDLGISKTAYMAVSDSPVSEYINGGEIEQFRNGIEARRNSMRNQLKYHSSNRSGHGRSTKLIPLEKLRAKVNNFKELTNHRYAKFIVDTALKNRCSIIQMEDLSGISKTDTFLKRWSYSNLQEKIENKAKTKGIEVKKVSPKFTSQRCNKCGYIDKESRKSQEKFECVNCGHKTNADLNAARNLSMLDIEKVIKAQCKAQKIKH

SEQ ID NO: 147 - protein, 450 aa, unidentified
MEGEKEKYVVQSYGIKLIKPVGVDEGDWDFAGKVLRDLDYICFRVKNKAATRTYMNVIEKLEYENVHGKGTYDKYFKTRYGKTFSAYNMEQAKEDKELNNGSFLREHFDSMAREGEKVVKNNMKKILNGSASNITFKRNQPIPIRSRMIFITKESGKYYAELTLLSPEKAKELDRKGRKGTRIKFLLSSKGQEKVILDRLTSGEYDLRDSHIHVKKKGNKLNNYLIVAYRLKVKDDKDLIPNKVLGVDLGISKTAYMAVSDSPVSEYINGGEIEQFRNGIEARRNSMRNQLKYHSSNRSGHGRSTKLIPLEKLRAKVNNFKELTNHRYAKFIVDTALKNRCSIIQMEDLSGISKTDTFLKRWSYSNLQEKIENKAKTKGIEVKKVSPKFTSQRCNKCGYIDKESRKSQEKFECVNCGHKTNADLNAARNLSMLDIEKVIKTQCKAQKIKH

SEQ ID NO: 148 - protein, 450 aa, unidentified
MEGEKEKYVVQSYGIKLIKPVGVDEGDWDFAGKVLRDLDYICFRVKNKAATRTYMNVIEKLEYENVHGKGTYDKYFKTRYGKTFSAYNMEQAKEDKELNNGSFLREHFDSMAREGEKVVKNNMKKILNGSASNITFKRNQPIPIRSRMIFITKESGKYYAELTLLSPEKAKELDRKGRKGTRIKFLLSSKGQEKVILDRLTSGEYDLRDSHIHVKKKGNKLNNYLIVAYRLKVKDDKDLIPNKVLGVDLGISKTAYMAVSDSPVSEYINGGEIEQFRNGIEARRNSMRNQLKYHSSNRSGHGRSTKLIPLEKLRAKVNNFKELTNHRYAKFIVDTALKNRCSIIQMEDLSGISKTDTFLKRWSYSNLQEKIENKAKTKGIEVKKVSPKFTSQRCNKCGYIDKESRKSQEKFECVNCGHKTNADLNAARNLSMLDIEKVIKAQCKAQKIKH

SEQ ID NO: 149 - protein, 450 aa, unidentified
MEGEKEKYVVQSYGIKLIKPVGVDEGDWDFAGKVLRDLDYICFRVKNKAATRTYMNVIEKLEYENVHGKGTYDKYFKTRYGKTFSAYNMEQAKEDKELNNGSFLREHFDSMAREGEKVVKNNMKKILNGSASNITFKRNQPIPIRSRMIFITKESGKYYAELTLLSPEKAKELDRKGRKGTRIKFLLSSKGQEKVILDRLTSGEYDLRDSHIHVKKKGNKLNNYLIVAYRLKVKDDKDLIPNKVLGVDLGISKAAYMAVSDSPVSEYINGGEIEQFRNGIEARRNSMRNQLKYHSSNRSGHGRSTKLIPLEKLRAKVNNFKELTNHRYANFIVDTALKNRCTIIQMEDLSGISKTDTFLKRWSYSNLQEKIENKAKTKGIEVKKVSPKFTSQRCNKCGYIDKESRKSQEKFECVNCGHKTNADLNAARNLSMLDIEKVIKAQCKAQKIKH

SEQ ID NO: 150 - protein, 450 aa, unidentified
MEGEKEKYVVQSYGIKLIKPVGVDEGDWDFAGKVLRDLDYICFRVKNKTATRTYMNVIEKLEYENVHGKGTYDKYFKTRYGKTFSAYNMEQAKEDKELNNGSFLREHFDSMAREGEKVVKNNMKKILNGSASNITFKRNQPIPIRSRMIFITKESGKYYAELTLLSPEKAKELDRKGRKGTRIKFLLSSKGQEKVILDRLTSGEYDLRDSHIHVKKRGSKLNNYLIVAYRLKVKDDKDLIPNKVLGVDLGISKAAYMAVSDSPVSEYINGGEIEQFRNGIEARRNSMRNQLKYHSSNRSGHGRSTKLIPLEKLRAKVNNFKELTNHRYAKFIVDTALKNRCTIIQMEDLSGISKTDTFLKRWSYSNLQEKIENKAKTKGIEVKKVSPKFTSQRCNKCGYIDKESRKSQEKFECVNCGHKTNADLNAARNLSMLDIEKVIKAQCKAQKIKH

SEQ ID NO: 151 - protein, 442 aa, unidentified
MATKCIKLAGEYVKENSLEKDKFFKELRDIQYKTWLACNRAITYYYSNDMQNFIQKDVGIPKEDDKLLYGKAFKSWVANRISEILGDGISSYATDCISQFVSNRYKNDKKAGLLKGNVALSQFKRDIPVMLRERAYSIIDTPKGLGIEISFFSKTKQQELGIKRILFTFPKIDGSSKSILTRIMDKTYKQGSIQITYNKRKKKWMFAISYTFENKLEKVLNDNLVMGIDLGITKVATMSIYDIEKHQYKNMCFKEQTIDGTELIHYRQKIEARRKSLSISSKWASDNATGHGYKRRMKKANNIGDKYNRFKDTYNHKVSRYIVDLAYKHGVKTIQMEDLSGFSEHQSESLLKNWSYYDLQNKIKYKAEEKGINTIFINPQYTSKRCSKCGNIHEENRDCKNNQAKFECIICGHKENADINASKNIAIPYIDKIIKEYIKDTI

SEQ ID NO: 152 - protein, 442 aa, unidentified
MATKCIKLAGEYAKENSLEKDKFFKELRDIQYKTWLACNRAITYYYSNDMQNFIQKDVGIPKEDDKLLYGKAFKSWVANRISEILGDGISSYATDCISQFVSNRYKNDKKAGLLKGNVALSQFKRDIPVMLRERAYSIIDTPKGLGIEISFFSKTKQQELGIKRILFTFPKIDGSSKSILTRIMDKTYKQGSIQITYNKRKKKWMFAISYTFENKLEKVLNDNLVMGIDLGITKVATMSIYDIEKHQYKNMCFKEQTIDGTELIHYRQKIEARRKSLSISSKWASDNATGHGYKRRMKKANNIGDKYNRFKDTYNHKVSRYIVDLAYKHGVKTIQMEDLSGFSEHQSESLLKNWSYYDLQNKIKYKAEEKGINTIFINPQYTSKRCSKCGNIHEENRDCKNNQAKFECIICGHKENADINASKNIAIPYIDKIIKEYIKDTI

SEQ ID NO: 153 - protein, 441 aa, unidentified
MATKCIKLAGEYAKENSLEKDKFFKELRDIQYKTWLACNRAITYFYSNDMQNLIQKDVGIPKEDDKTIFGKSFVAWVENRMNEIMEGISSANVAQTRQFVNNRYSQDKKNGLLKGSVSLSQFKRDLPIIIHNKAYNVIETSKGLGVEISFFNKEKQKELNVKRIKLLFPKLDNSSKQILIRLMDKTYKQGSIQVTYNRRKKKWMFAISYTFENKLEKVLDDNLVMGIDLGITKVATMSIYDIEKHEYKKMYFKEQTIDGTELIHYRQRIEARRKSLSIASKWASDNATGHGYKRRMKKANNIGDKYNRFKDTYNHKVSRYIVDLAYKHGVKTIQMEDLSGFSEHQSESLLKNWSYYDLQNKIKYKAEEKGINTIFINPQYTSKRCSKCGNIHEENRDCKNNQSKFECVVCGHKENADINASKNIAIPYIDKIIKEYIKDTK

SEQ ID NO: 154 - protein, 442 aa, unidentified
MATKCIKLAGEYAKENSLEKDKFFKELRDIQYKTWLACNRAITYFYSNDMQNLIQKDVGIPKEDDKLLYGKAFKSWVANRISEILGDGISSYATDCITQFVSNRYKNDKKAGLLKGNVALSQFKRDIPVMLRERAYSIIDTPKGLGVEISFFSKTKQQELGIKRILFTFPKIDGSSKSILTRIMDKTYKQGSIQITYNKRKKKWMFAISYTFENKLEKVLDDNLVMGIDLGITKVATMSIYDIEKHEYKKMYFKNQTIDGTELIHYRQRIEARRKSLSIASKWASDNATGHGYKRRMKKVNNIGDKYNRFKDTYNHKVSRYIVDLAYKYGVKTIQMEELRGFSEHQSVSLLKNWSYYDLQNKIKYKAEEKGINTIFINPQYTSKRCSKCGNIHEENRDCKNNQAKFECVICGHKENADINASKNIAIPYIDEIIKEYIKDTI

SEQ ID NO: 155 - protein, 450 aa, unidentified
MKINKCIKVTLIKCLNYDYKEIKQIIRDFNYTACKASNKAMRMWLFHTQDMIDKRNEDKLFNQIQYEKDTYGKSYRNVIEGEMKKLMPLANTSNVGTLHQQLVQNDWGRLKKDILSCKANIPNYKINTPYFIKNDNFKLRNHNGYFVGIAFFNKEGLQQYGYKIGHKFEFQIDKLDGNKKATINKIINGEYKQGSAQISISKKGKIELIISYGFEKEEIPVLDNNRILGIDLGITNVATMSAYDSIKDEYDYFSWKTNVISGKELIAFRQKYYNLRRDLSIASKTAGKGRCGHGYKTKMKPVDKIRNRIANFADTYNHKISKYIVEFAVKNRCGVIQMEDLSGATSEVHNKMLKDWSYYDLQQKIEYKAREQGIEVKKVNPKYTSKRCNKCGCIHEDNRDCKNNQAKFECKVCGHSENADINASRNIAIPEIDKIIDKTEILHSENRQAS

SEQ ID NO: 156 - protein, 436 aa, unidentified
MKFNKCIKVTLIRCLNYDYRETKQIVRDFQYKYSKAYNMATNYLYLWDTNSMNLKNLYDTKIVDKELLGKSKCAWIENRMNEIIQDSNTSNVAQARQDVVNKYNKCKKDGLFKGKVSLPTYKLDSKVIIHNNSYKLRNHNGYFIDIGLLNKDKQKELNVGRFEFQIDKLDGNKKATINKIINGEYKQGSAQISISKKGKIELIISYSFDKEEVPVLDKNRVLGIDLGITNVATMSVYDSIKDEYDYFSWKTNVISGKELIAFRQKYYNLRRDISIASKVAGKGRCGHGYKTKMKPVDKIRNKIANFSDTYNHKISKYIVEFAVKNNCGVIQMEDLSGTTADTNNKMLKDWSYYGLQQKIEYKAKEQGIEVKKVNPKYTSKRCNKCGCIHEDNRDCKNSQAKFECKVCGHKDNADINASKNIAIPDIDIIIKETEIL

SEQ ID NO: 157 - protein, 493 aa, unidentified
MNSIKINKCIKVTLKKCLNLDIKEVKNIIKDMNYLACKASNKAIKMWQLHTEEMMERKFEDKNFNISQYEKDTYKKSYRNVIEGKMKDIMDICNTSNVGTLHQQLVQNDWGRLKKDVLNYRANVPTYKLDTPYFIKNHNYKLYNNGGWFVDIAFFNKQGLIQYGYKVGHKFEFEIDKIDNNKKSTITKIINGEYKQGSAQISLSKKGKIELIISFSFEKELKNNLDKNRILGIDLGIVNTATMSIWDNNKQAWDWVDYKENRIDGKELIKFRQKLFSMGMSNSEIEKEIFKANSKIKENQLRKRELGVIDGLELAKYRDTVYKKRREMSIASKYAGKGRSGHGRKTKMKPVDKIRNKVYNFADTYNHKYSKYIVDFAVKHNCGIIQMEDLSNATANKKEKFLKDWSYFDLQTKIEYKAKEYGIEVIKINPKYTSKRCSKCGGIHIDNRDCQHNQANFECKICGHKENADINASRNISIPYIDKIIAKTKVQAD

SEQ ID NO: 158 - protein, 490 aa, unidentified
MVTKVVECKIIGFDSLLFEKEDVTENAKKDNKKAKEAWKRNIEMIDQAVRDFVAAKNRAITYLFTEYIKVRDAMATSGESSFSSVWKKMHDEKGRSKAMYHYLVKALPGYLTGNVSEGSRRLEKRFDQAVKDGLLYGRVSLPSFRDNAALDFRGDSVKIKQEIIKGEEKLYVELNLKRGIKLHCNIGFSGNKGAKAVAERIGKGEYKAGTASIVKRKSRYFIQICYSFDSDIYMRSNGMVGLAEGLDPKRTLGVDLGLARPVAMQPYDSDSRKYLSGGKNDEFVNTERYTNRFTISGDRIAAFRKQVEKKKSVLQRDLRDVSPGHTGKGRIKRIKPVEKFKDRIANFRKTTNFVYAKRIVDTAIKYHCGTIRIEDLTIDVSSRKSERFLKTDWTYYDLQQKIESRAANFGIVVQKVAPQYTSQKCSKCGHVSADNRKDQSHFKCVSCGFEMNADLNAARNIAALSPNDKIKKEKNKEAKDGQMQLEFILN

SEQ ID NO: 159 - protein, 483 aa, unidentified
MVTKVVKCKVVGFDSLPFEKKDAAGNAKINNKKAKEAWNRNIEMIDQAVKDFVAAKNRGITYLFTESIKIQDAMDASGESSFYDAWKKMHGGTQLRTTMDHFLVKELPGYLAENVCESSGRLVTRFNQAKKDGLFKGCVSLPSFRDNAALDFRGSSVKIKQETINGEEKLYVELRFKRGIKLHCNIDFSGNNGAKAIAKRVCNGEYKSGTASIVKRKGKYFIYICYSFEPETYMRLNGMDGLAEKLDTKRILGVDLGLAKPVAMQPYDSISQKYLSGGKNDEFVNTEEYTNRFTISGDQITAFRKQVEKRKRALQRDLRDVSPGHTGKGRIKRIKPVETFKDRIANFRNTTNFIYAKRIVDTAIKYHCGIIRIEALTIDVSSRKSERFLKTDWTYYDLQQKIESRAANFGIVVQKVKPNYTSQKCSKCGHVSADNRKDQSHFKCVSCGFEMNADLNAARNIATLSPTDKINKKKKREVKDDQV

SEQ ID NO: 160 - protein, 419 aa, unidentified
MTKTKMITKATRFQIIKPLDTGWDELGQVLRDLSYHTTKLCNMAVQLYWQHHNYRLAHKEETGKYPAAAEDKERYGCSFRNHVYRKMREMYPDMASSNTSQTNQFAMSRWQNDVREIMRLQKSIPSFRLGTPAQVANANYTLSVEEQDNGRSSFIATITLLSLAAGKQTRYSILLDGGDRSKKAIFRRIMEGEYKQGAMQITYNKRKKKWFCIVSFSFIPEKQNRELDPDRAMGLMFGTGNYTVFAAYSVGQKRYTLPAGEVIATEKKIHAIAERRKEIQRHAGYRGHGIKRKLQGTEVLAGQASAIRDTLNHKYSRRVVDVAMANRCGTIKILDKLVTPGASVSELVNYPWAGVVEKIKYKAEEKGIAVKVVSLDGTRCHKCDAEIEIDSDNESPLTCPSCGAKIDKDYNTAKRIAKS

SEQ ID NO: 161 - protein, 411 aa, unidentified
MITNTCKLEVSFIDTKFSDLFAMINATTQVKRYATDRITEWKSFSSRYHEENGEYPKLEDIYGFKSIEGLIYHELSKKKTGLSSRSLIATIHSCFLSYKQASKKHSHPNWSDSQPIIMQGKYLNLHHESGNYIVDYPLFSKQYRVEHEITHILRMRLNTKENHHKNILDRILSGEYHLCQSQLKYVKKQNKSHWYFLLAYSHKSEGNPNIDPTRVLGVFMSENVALYASSKRLPMVFKIDGGEIPTFASRQEAIIRSKQRQAACCGDGRIGHGYKTRTKSIYPNKQTLAHFRETINSRYAKALIDFAKTNGFGEVRLEDLKGIKADREFPRFLIHWTYFDLQEKIKNAARKCGIKVTLVSSETIGITCSSCGHIDPNNRDRGVKNRFVCTKCNIVKQTDWNASQVLSSLEI

SEQ ID NO: 162 - protein, 439 aa, unidentified
MLTKCIKIPIEYSKDNILKSEEFYKELRDIQYKSWRACNRALTYFYMHDMENIMLKESGKDIKSDKELYGKTYGSWIENRMNEIMKGVLSNNVAQTRQYISNRYGQDRKNGLLKGNVSLSEIKRNMPIIIHAKAFKIIDIIKGLGVEVSFFNLNKQRELGVKKIKFLFPKIHSSEKSILKRLVDKSYKQGIAQLSFNERKKKWLLTISYSFEKRIEELDEDLVMGIDLGISKITTFSILNAKTKEYIQMNFKDKFVDGKELIHYRQKLEARRRELSIASKYTSKNNLGHGYKTKMQSVNMAGDKYNRFKETYNHKVSRFIIEIALRYRVKNIQMEDLSGFSEHQTESLLGNWSYYDLQNKIAYKAEELGINIIFIDPKYTSQRCNKCGNINSKNRNCKENQEKFECIRCGYKENADINASMNIAIPNIENIINNTIKIK

SEQ ID NO: 163 - protein, 475 aa, unidentified
MKEKYFVKVAKFEILKPANGMTWKEFRQLLMSVRYRVFRLANLCVSENFLQFHLWRKKEIDKIPTLKISELNRQLREMLLEEKKTSDAEQTRICKRGALPASIVDALSQYKIRALTAKSKWRDVIRGNASLPSFRNDAAIPICCHKPSHRRLEKTSNGNVELELMICMKPYPRIILKTEKISGNMKATLERLLANRGNSDTGYQQRFFEVVQDRQEGRRWYLHVTYKFPASLLRLNPQVIVGIDLGFSCPLYAAINNGLARLGWRHYESLGKRIRNLQNQVFARRRSMQSGGNASLTMDTARGGHGRKRILRPIEKLAGRVNNAYSTLNHQLSRSVIEFAKNHGAGVIQMENLEGLKEQLTGTFLGSRWRYHELQQFIEYKAKEVGIEVRKINPQYTSRRCSECGYINIKFDRAFRDANRKDGKTAKFICPKCKWEGDPDYNAARNLATLDIENLIRQQLINQGIPSEREETPVL

SEQ ID NO: 164 - protein, 422 aa, unidentified
MQECKYVQRSIKLKIVCPLDENITWEEFGFLLRGLSFKVARASNFCMLHHLLYAMKLETVHLNRKGGLYCYPYLAEEYPEVPAGILCAAETRARKLFKQHAVEILRSDRALPNFRKDVGIPVPAASYKIMQDGKGDFLMEIQLLSRQAAKTGKLSGRICLALATNWRDKTAVAALQKIAEGSLKTGVGTLFREKKNWYFVVPYQREKGLSKEATENDECVMGVKLGVQNALVYAFDRSLKRGSLSGEEVKARQEQFYARKQNILEQYKWSGRKGHGRERALKPIQELYEKERNFRNTANLRYANWIVEIAVKNRCGEIHLESGKGVGFGQNSIMLHFWPVWDLKNRIKQKAEEHGIKVVECNVPDIWGACSECGAKSGNSVEKQSFSCPSCGYGKQEEKYGVGYVSAEYNAARNLALWRKEE

SEQ ID NO: 165 - protein, 438 aa, unidentified
MGKVHTRTIKLKLIVSTEDKNVAWKRIRQISNDAWRAANWIASGQLFNDQLVRRIYARRKINPREDSEAVEQIEKEFEAFFGTKRQATTEWDIKEAFPDLPPYVTNPLNHVVVASYKKEKPDMLSGNRSLRTYKRGMPIQTAKAAINFSKNETGQHFVKWTLGRKEFIDFEIYYGQDRANNRLTIDRIINGQIDYSAPMIQLKDKNLFLLLPVKEPEIAADLDPDIALGVDLGVATPAYMALSKGPARRAVGDKEDFLKVRLQMQSRKRRLQRSIKSAHGGKGRQKKLKALNQIGEKERQFARTYNHYISREIVNFAIRHNAGTIKLEMLEGFGQEEQQAFVLRNWSYFELQTFIEYKAKKVGIKILKIDPYRTSQTCSSCGHFEEGQRKDQSTFECKNCGEKLHADYNAALNIARSKKIVTKKEQCEYYLNRNQNTI

SEQ ID NO: 166 - protein, 467 aa, unidentified
MADQGKTLFKVMPLRILKPVGDTRWDALGQMLRDTRYRVYRLANLAISEAYLGFHLFRTGKAEQFKTDTIGQLSRRLRQMLLDEKVPRDNLDRSSMTGAVPDTVASGLHQYKIRSVTNVNKWQQVVRGKSSLPTFRADMAVPIRCDKAGQRRLERNTQGDVELDLMICRRPYPRVVLATGTLGPGQQTILDRLLENEDNAKSGYRQRLFEVKEDSQTHQWWLYVTYEFPAPVLPVAHGDIVVGIDLGVSVPLYAAINNGHARLGRRQFQALGYRIRTLQNQVIMRRRAIQRGGRVGVSQPTARSGHGVHRKLLPTEKLRRRIDKSYTTLNHQLSAAVIDFAKNHGAGVIQIEDLSTLKEQLVGTFLGGRWRYHQFQQFLAYKAKENGITLREVNPKYTSRRCSECGFIHVDFDRAFRDRHHTEGMVTKFVCPQCKYEADPDYNAARNIATLDIEQRIKVQCQVQKLM

SEQ ID NO: 167 - protein, 543 aa, unidentified
MAKKQDDNTMTIARKITLIPVASERKEWKKRIDAFLEKDFPMQIEIKKKQIKNTSKPERVDGYKQQLAELEKQYEEFKENGIKEYTHKMASDYTYDIVRRAMESEARRKNYILSYIYTKMIQDEVANLPTLTEKNKWVSANVKECYRKAGNKNGSIFTNVDIDNPLAGYGSDFGQAFTRKIKKLIKDGILEGNVSVPNYKLDSPFALSNQNFGIFTDCENITELKKNIGKPTYPVYVCLGKHGLPTIAKFKINFGHKQNKNKAELISTIIKILTGEYSVGGSTFGIDDDKIEMNLSITMQKQKMDLDENTVVGVDLGLAVPAVCALNNNEYDKQYIGSGNDLVTRRTKFQNEYTQLQKALKLAKGGHGRKRKLLALERLKEKEKNFVDTYCHRVSKKVVDYAIKHRAKYINIENLKGYDSSEFVLRNWSFYKLQQYITYKAEQCGIEVRKINPSFTSQVCSFCGHWEEGQRKDQATFKCKNPNCKSHKLYTVNADYNAARNIAMSTQFTDDKFKCSKKTIQDAADYYGIVLEDDKNNDNKKAA

SEQ ID NO: 168 - protein, 487 aa, unidentified
MLLIQIEEVNQMITSRKIKLAIVSDNKDTAYSFIREETRNQNRALNVAYTHLYFEYVAQEKLKQSDKEYQQHLEKYKNAAAKKYQEFLTIKEKSKSDENLQPKMDKVRETYNKAMEKVYKIEKDYSKKAREIYQQSVGLAKQTRLGKLIKSEFDLHYDTVDRIGSNAMSDFSNDRKSGILSGERSLRNYKKTNPLMVRARSMKLYEEDNNFYIKWINDIVFKIIISAGSKQRMNIAELKSVFIKLLSGQCKMCDSSISLDKGLILNLSIDMPITKENVFIPNRVLGVDLGLKIPAYVSLNDTHYIKGAIGNIDDFLKVRTGLQSQRRRLQKSLQSTGGGKGRRKKLQALERLKTKEKNFVNTYNHFLSKNIVQFAVKNNAGAIHMEELKFDKMKNKSLLRNWSYYQLQTMVEYKAKSEGIEVYYVDASYTSQTCSKCGNLEEGQREARDTFVCKKCGYNVHADYNASQNIAKSTKAINKTIEITNIV

SEQ ID NO: 169 - protein, 417 aa, unidentified
MPKQEEEGMIITRKIEIACRNKDQYPLLQDYLKQCRLMANKAMTNYYILWQEMLETKPKNQTQKDYFLKKHGCSFQNTGYRYLRRCFGGMPSYLRRDVANRVYQDFRADLKNGLLKGERSLRNYRTGHLPLHNKDIQLCKEETSSDYLLRWRMGIEFVLLFGRDRSGNRIIADRLIEGTYRVSGSQIYKDWRKKKWFVLLCVKIPQQDNNLDETAVVGVDLGLSTPAVCALSNGMARAYIGSSNLLKRFEMQKKQRSRQREFIPATNKHGRQKVVKAIYAMKDKERRFVHSFNHAISKKIIQFALKYKAAVIRMEDLSGYNGDETVLRNWSYYELQSMIEYKAEKEKIRVEKIPACYTSRACSQCGFIDKENRKTQAQFECVKCGFKENADYNAALNIARGGVKIPEENQEDFGENE

SEQ ID NO: 170 - protein, 424 aa, unidentified
MILTRKIQIIPLGEKEEIDRVYKYLRDGIFYQNKAMNQYMSALYIAAIKDISKEDRKELNRLYSRVSNSKKGSAYDKSIEFAKNMNLGYVVKQVKQDFANSCKNGLLCGKVSLPTYRKNNPLLVHVNFVRLRSTNYHQDNGMYHNYESHTDFLDHLYSKDLEVFIKFANNITFKMIFGNPHKSAYLRSEIQQIFEENYKVCGSSIQIDGKKIILNLSMDIPKQELELDENIVVGVDLGLAIPAMCGLNTNDYIRQSIGSKDDFLRIRTQLQSQRRRLQKSLASTSGGHGRQKKLKPLEKLKDRERNFVKTYNHYVSKNVVDFAVKNKAKYINVEDLSGFDSNQFILRNWSFYELQQFITYKAAKYGIEVRKINPYHTSQICSCCGHWEEGQRIDQAHFKCKSCGAELNADFNASRNIAMSTDFV

SEQ ID NO: 171 - protein, 416 aa, unidentified
MQRSIILKILRPYDESIKWEELGYLLRGLSYKVCKISNYCMTHHLLRALNLETENLNPQGHLYCYPRLAQDYPDVPAGILCAAEGRARKVFLKCGKEVLRSETALPNFRKDCSIPIPVAGYSLLKAGEDTYVANVQLLSRQAAKTQKLPGRIQLVLAKNWRDRSAGPVLQQLTEGTLKRGIASLFRRKRDWYISIPYEAEAQKTEDGSFAPGLVMGVILGTQCALAYAFNRSPKRGAIGGEEILAHKEKFRARKKHIREQYNWSGRKGHGRGSALKPLQALYEKERNYRNLTNERYAKWVIEIAKKNRCGKIHLDGGSAGGTGTPHVMLAYWPQGELRKKIRYKAEACGIEVVECDGRDVWNRCSKCGAVQEVSGERRWFTCSHCGYGKDDKKTSSSFVTVDYNAARNMAIMESKP

SEQ ID NO: 172 - protein, 488 aa, unidentified
MATEYTCITRKIEVHLHKHGDTEEAAQRLKDEYHIWDNINDNLYKAANRIVSHCFFNDAYEYRLKIHSPEFRQIEKSLQHAKKNKLSEDDIKKLKAERKSLCADFREQRLAFLRGGATEGANPEQNSTYQVVSHEFLDVIPSDILTCLNQNIASVYKQYALEVEMGKRTIPNFKKGIPVPFAIKQNKQLRLRKRNDGSVYVLLPRELEWDLSFGRDRSNNREIVERVLSGQYDVGNSSIQETRSGKRFLLLVVKIPKASRTLDTKKVVGVDLGIATPLYAALNDNEYGGLSIGSQEQFLKIRMRMTAQKRELQRNLRYTTNGGHGRTQKLQALERLEGKERNWVHLQNHIFSKSIIEYALKNNAGVIQMERLTGFGHDKNDEVGTEYKFILRYWSFFELQSMIEYKAKAAGIEVRYINPYHTSQTCSFCGKYEKGQRINQATFVCKNPDCVKGKGRQKSDGTYEGINADWNAARNIALSEEFVDKKKK

SEQ ID NO: 173 - protein, 482 aa, unidentified
MNTVRKIKLTILGDIETRNKQYKWIRDEQYKQYRALNLCMTYMVTNLMLRNSESGLENRKEKEILKIQNKIKKDEEKLEKELKKEKSKEEKIQDIKFNIEELKLQKEKLENELRNIKEYRSNIDEEFKKMYVDDLYNVLNKVPFQHEDMKSLVTQRIKKDFNNDVKEIMRGDRSVRNYKRNFPILTRGRDLKFQYIEKSEDIEIRWIEGIKFKCILGKTSKSLELKHTLHKVINKEYKVCDSSLQFDKNNNLILNLTLDIPQDNKYEKITNRVVGVDLGLKIPAYVALNDTKYIRKAIGSIDDFLKVRTQMQSRVRKLQKSLQTARGGKGRNKKMKALDRFREKERNFARNYNHFLSYNIVKFALDNKAEQINLELLEMKETQNKSILRNWSYYQLQSFIKYKAERIGIKVEYIDPYHTSQICSECGNYEEGQRVEQATFVCKRCGHKINADYNAARNIAMSKEYISKKEESKYYKNNKNMV

SEQ ID NO: 174 - protein, 450 aa, unidentified
MSKNTYTITRKIQLIPVGDKEEVNRVYTYLRDGMKAQNMALNQYISALYFAMQNDATKDSRKELRNLFSRISTSKKGSAYDDTIEFAKGLPSCMMTRKVESDFKNAMKKGLKYGKLSLPTYRDTNPLLIHIDYVRLRSTNPHLDNGLYHNYKNHTEFLEHLYDENLELFIKFANKITFKIILGNPHKSSELRSVFKNIFEDCYHIQGSSIEIEKNKIILNMSMSIPVKKIELDENIVVGVDLGMAIPAVCALNTNEYIHKSLGNYNDFIRERTKIQAQKKRMQKSLTYTNGGHGRKKKLKPLNRFKERERNWVKTYNHKISKQIIDFAIKNKAKYINLEDLSGIAKEDKDKFVLRNWSYFELQQFITYKAEKYGIIVRKIKPEYTSQKCSCCGHLEENQRINQSEFICKNPNCKNFGKIVNADYNGARNIAMSTNFVEEKETKSKPKKVA

SEQ ID NO: 175 - protein, 416 aa, unidentified
MQRIIILKILRPYDESIKWEDLGYLLHGLSYKVCKISNYCMTHHLLRALNLETENLNPQGHLYCYPRLAQDYPDVPAGILCTAEGRARKVFQKCGKEVLRSETVLPNFRKDCSIPIPVAGYSLLKAGEDTYLANIQFLSRQAAKTQKLPGRIQLVLANNWRDRSAGKVLQQLTEGTLKRGIASLFRRKRDWYISIPYEVEAQKTEDGSFAPGLVMGVILGTQCALAYAFNRSPKRGAIGGEEVLAHKEKFRARKKHIREQYNWSGRKGHGRGSALKPLQALYEKERNYRNLTNERYAKWVIEIAKKNRCGKIHLDGGSAGGTGTPHVMLAYWPQGELRKKIRYKAEACGIEVVECDGRDVWNRCSKCGAVQEVSGERRWFTCSHCGYGKDDKKTSSSFVTVDYNAARNMAIMESKP

SEQ ID NO: 176 - protein, 472 aa, unidentified
MATITRKIELYIDKSNLTDDEYKAQWQYIRQIDNTLFLAANRISSHCLLNDELEIRLKLQMPEYCEIEKSLRNSKKNKLSKEEISELKLRRKELDAVVKRQKEEFLKTSQNSTYQLVAYEFTNIPTEILTNLNNDIVGKYGKARLDIIKGIKSPSTYKKGIPIPFSVNKKSPFVFIDGDLEWFKGKTKETTGKILRFKLHFGKDKSNNRAIVERLVESAKLGKKKGEAYIVNNSSIQLVEKENTTKIFLLLSLDIPTTKRALDSNLVMGIDLGINYPIYYATNGNAYIKGHIGDRDSFLNERMVFQRRFRELQRLQCTQGGRGRLKKLAPLEKLREKERNWVRTKNHIFSREIIKCALKIGAGTIHLEKLKNIGKDKDGNIEDSKKYILRNWSYHELQEMIEYKAAMEGITVKYVNPAYTSQTCSCCGNRGERISQSVFKCLCPECSEYGKEVNADYNAARNIAKSEIFVKK

SEQ ID NO: 177 - protein, 543 aa, unidentified
MDNTITITRKYTLIPTFSDTKEWTKKVMEYTKTSYIEKIKYYEEKIKKTKKKDKEEREKYENRLSQLKEQQLDFEENGTLLQTNVNDYTYDLVREAMASESDRKNMIISYVCGELINRDAKDMDFKERNKLISELCNYGYRVKGSKKGSLFDHLDIDNPLGGYGVSFCQDLTKKIKELVNNKRWLDGKVSTLHYKDDSPFSIAKATMGFAHDYDSFEELCEHIREKDCNLYFNFGNNGKPTIARFKINLGANRKNKDELISTIIRVYSGEYQYCGSSIGIEGTKIILNLSMKIPKQEKELDENTVVGVDLGIAVPAVCALNNNVYARKFVGNKDDFFKVRKQLNAQYKRVQSALKRASGGHGRKKKLKALERLRKKEAHFVETYCHMVSKAVVDFALKYNAKYINLENLTGYDTDDIVLRNWSYYKLQQYITYKASKYGIEVRKINPCYTSQICSECGNYHPENRPKGDKGQAYFNCHNKECITHGKKSPYQYGINADFNAARNIAKSTLWMGKGKVTEESKKKAREYYGIEEEYEELNKEVA

SEQ ID NO: 178 - protein, 469 aa, unidentified
MITARKIKLTIAENREEGYSFIRKELQEQNKALNMAINHLYFNYVAREKIKLADETYKVKLEEGECYLERKYIELKEAKTDKQKENIKKSIEATKKKLETLRKVENKEVSNNFKEIIATSEQINLRDLISNNFNLKSDTKDKLTQKVVQDFYNDIVGVLRGERTLRRYKKDNPLYIRGRSLTLYREGEDYYIKWMNGIVFKCVLGVKKQNSLELQKTLDKVIFEEYKLCDSSIGFKDNKLILNLTLDIPVSNTNKFEKVIGRIVGVDLGMKIPAYCALNDSEDVRKAIGSIDDFLKVRTQMRSRRRKLQRALKSTNGGQGKNKKLSALNSFEAKEKNFAKTYNHFLSSNIIKFATDNKAEQINMEFLSLSETQNKSVLSNWSYYQLQQMIEYKAERIGIKVKYVDPYLTSQTCSECGHYEDGQREVQSEFQCKKCGCKTNADYNASRNIAKSDKYITKKEESEYYKNKI

SEQ ID NO: 179 - protein, 478 aa, unidentified
MPILTRTIELIPIGDKEERDRCYKWIRDFMEEQSKMMNQYMSALYIAAVEEVSKDDRKELNNLYNRIATSKKGSAFSKEECNLPKGLGANYGQRVRSDFDTACENGLLHGRVSLPTYKKNFPIILAPIYVNLQKNNIEEKGKSAGFYHNYASYNELYDALKEENKPEIIWNFVQKMQYQIKFGNPYKSAFLRDEILHFLEGEYKAVGSQLSINSRGKIILNLSLDVPQKKVKLDENIVVGIDIGLAVPVMCAINNDYYKRLAVGDFEAFTRMREKLYSQKCKLQRQLKYTSGGHGRKKKLASLNAIRDREHRFVHTMNHKYSSEVINFALKNNAKYINMEDLTGFGKDNKGNAIDDYQFVLRNWSYFELQKMIQDKAQKYGIVVRKVESAYTSQLCSCCGEMGERVSQSVFRCLNPNCISHNKYEKQRKSGVGNYHFNADFNAARNISMSTNYTKKKRKTKAEKVEERKKNAIEKTAG

SEQ ID NO: 180 - protein, 425 aa, unidentified
MVKMKKETKQKRNTLCKKITLYPLGSKEEVTRVYNFIRDGQYMQYKGLNLIMGQLASQFYKCNSELDDPQYKEWVEQEIRNTNSLLQAMEFPKGIDSRSLIIQRVKQDFSSALKNGLARGERSITNYKRTLPLMTRGRDLKFSCNYSELEELEYKCFSKDFKVYVSWVNKIKFKVVLGSVKRSLALRKELCQILRSVYSIQGSSIEIKDKKIILNLSMGIPLMEKVLDENTVVGVDLGIAVPAVCGLNTNKNTRRFIGSKDDLLKIRTKVQSQRKSLNKKLRECTGGHGRSKKLQALDRLSESETNYVKTYLHMVSKEIIRFAVAHNAKYINIEKLEGYDASSFILRNWSYYQLQSFIEYKAKIKGIIVRKVNPYHTSQICSYCGHWEEGQRLSQDKFKCKCCGVELNADFNASRNIALSTEFVE

SEQ ID NO: 181 - protein, 437 aa, unidentified
MAKDTFTITRKIQLVPVGDKTEVNRVYNYIREGMKAQNLAMNQYISALYLGMQNDVSKDDRKELNNLFSRISTSKKGSAYDESIQFAKGLPIGSMTRKVKSDFDTAMKKGLKYGKLSLPTYKDSNPLLVHVDYVRLRSTNPHQDSGLYHNYTNHTEFLEHLYKSDFELFIKFANYITFKIILGNPHKSAEIRDVFKNIFEECYAIQGSSIGIYNNKIICNLSISIPKKQLCLDENIVVGVDLGLAVPAVCALNTVPYIHKSLGNYDDFVRERTKMQSQRKRLQKSLNYANGGHGRKKKLQSLERLKKRERNWVQTYNHKISKQIVDFAIKNKAQYINIEDLSGFDSSQFVLRNWSYFELQQFIEYKANKYGIIVRKINPYHTSQTCSFCGHWEEGQRISQSEFICKNPECVNHNKSINADYNAARNIAMSTDFVKNN

SEQ ID NO: 182 - protein, 517 aa, unidentified
MDNTITITRKYALIPEFSDRKEWKKRVYDFTINDLEQKIDYRNKKKQDASELESQLEYIKNGGDFTRNMVNNYTYSLVRTAMEEEARRKNYILSWIFSEMRANRVDQMESLKDKFKFVSDTINYAYRKAGSNKGSLFDETEIHCILKSYGIAFSQELTKEIKELVKNGVLEGKVVIPTYKLDSPFTIAKSHFSFEHDYDSFEELCEHISDSDCKMYMNYGGDNRKDGINPASIAKFKISIGHGKNKDELKSTLLKVYSGEYQYCGSSIQITKNKIILNLTMKIPKIETKLDENTVVGVDLGIAIPAMCALNNNMYERLAIGSADDFLRTRTKLQSQRRRLQKSLKNSNGGHGRNKKLKVLERLGKSETHFVETYCHMVSKRVVEFAVKNRAKYINIENLNGYDTSQFILRNWSYYKLQQYITYKAERYGILVRKINPCYTSQVCSVCGNWEEGQRKTQSSFECANPECKSHEKYKYGFNADFNAARNIAMSTLFMETGNVTEKSKEEARKYYGIEKD

SEQ ID NO: 183 - protein, 517 aa, unidentified
MDNTITITRKYALIPEFSDRKEWKKRVYDFTINDLEQKIDYRNKKKQDASELESQLEYIKNGGDFTRNMVNNYTYSLVRTAMEEEARRKNYILSWIFSEMRANRVDQMESLKDKFKFVSDTINYAYRKAGSNKGSLFDETEIHCILKSYGIAFSQELTKEIKELVKNGVLEGKVVIPTYKLDSPFTIAKSHFSFEHDYDSFEELCEHISDSDCKMYMNYGGDNRKDGINPASIAKFKISIGHGKNKDELKSTLLKVYSGEYQYCGSSIQIAKNKIILNLTMKIPKIETKLDENTVVGVDLGIAIPAMCALNNNMYERLAIGSADDFLRTRTKLQSQRRRLQKSLKNSNGGHGRNKKLKVLERLGKSETHFVETYCHMVSKRVVEFAVKNRAKYINIENLNGYDTSQFILRNWSYYKLQQYITYKAERYGILVRKINPCYTSQVCSVCGNWEEGQRKTQSSFECANPECKSHEKYKYGFNADFNAARNIAMSTLFMETGNVTEKSKEEARKYYGIEKD

SEQ ID NO: 184 - protein, 551 aa, unidentified
MNLHGYKTDAKGFSSFLQSGNPVNLHGCKTRKEIKMESTTVITRKYTLIPEFSECKEWKKRVYDFTVKDIEQRIEYKKKKKQDISDLEIQLECIKNNSEFTRSMVNNYTYNLVRTAMEEEARRKNYILSWIFSEMTANRVDQMESLKDKFKFVSSTINYAYRKAGSNKGSLFDETEIHCMLKSYGIAFSQELTKKIKELVKNGVLEGKVVIPTYKLDSPFTIAKAHFSFDHDYDSFEELCEHINDSDCKMYMNYGGNNTKTGTNPASIARFRINLGHGKNRDELKATLLKVYSGEYRYCGSSIQISKNKITLNLSMKIPKVEMKLDENTVVGVDLGIAIPAMCALNNNLYERESIGKVNDFTRVRKKHQAQVKRLQKSLKSATGSHGRSKKMRALNRVKKSEAHFVESYCHYVSRKVVDFALKHNAKYINMENLKGYDTSQFILRNWSYYKLQQYITYKADRYGIVVRKINPCYTSQVCSVCGHWEPDQRKTQASFECANSECESHKKYKYGFNADFNAARNIAMSTLFMEDGEVTEKKKEEAREYYGIKK

SEQ ID NO: 185 - protein, 468 aa, unidentified
MPTITRKIELTLCTEGLSDEQRKEQWKLLYHINDNLYKAANNISSKLYLDEHVSSMVRMKHAEYLSLLKELARAEKQQTPDEGLIAELSRKLSAAEKEMADQELAICKYATEMSTQTLSYNFAKEIETNIFGQILTCLRQGVYATFNSDAKDVKRGERAIRNYKKGMPIPFPWNNSLKIEADSGEFYLRWYNGLRFLLTFGKDRSNNRMIVKRCMKMDEDFEGEYKLCNSSIQLAKRDGKPKLFLLLVVNIPQEHVELNKKIVVGVDLGVNVPAYVATNITEERKAIGDREHFLNTRMAFQRRYKSLQRLKGTAGGKGRTKKLEPLERLRDAERNWVHTQNHLFSREVVNFAVQARAATIHMEDLSGFGKDKDGNADEKKEFVLRNWSFYELQNMIAYKAAKYGIKVVKIRPAYTSKTCSWCGQQGDRKSTTFICENPECKHYGESIHADYNAARNIANSNDIVKENE

SEQ ID NO: 186 - protein, 522 aa, unidentified
MDNTITITRKYALIPEFSDRKEWKKRVYDFTINDLEQKIDYRNKKKQDTSKLESQLEYIKNGGDFTRSMVNNYTYSLVSTAMEEEARRKNYILSWIFSEMRANRVDQMESLKDKFKFVSDTINYAYRKAGSNKGSLFDETEIRCILKSYGIAFSQELTKEIKELVKNGVLEGKVVIPTYKLDSPFTIAKSHFSFEHDYDSFEELCEHISDSDCKMYMNYGGDNKKDGINPASIARFRISLGHGKNKDELRATLLKVYSGEYQYCGSSIQITKNKIILNLTMKIPKIETKLDKNTVVGVDLGIAVPAMCALNNNMYERLAIGSADEFLRVRTKYQAQRRRLQKSLKNSSAGHGRKKKLKALDRMDKAESHFVETYCHIVSKRVVEFAVKNRAKYINIENLNGYDTSQFILRNWSYYKLQQYITYKAERYGIAVRKINPCYTSQVCSVCGNWEDGQRKTQASFKCANQKCESHKKYKYGFNADFNAARNIAMSTLFMEDGEVTEKKKEEAREYYGIKKENSKAV

SEQ ID NO: 187 - protein, 504 aa, unidentified
MSYTEQFYIDKIAYYKNKLTKTKGKEEKQKIKDKLASLEEQENEFEESGILTQANVTDYTYDLVRNAMASEANRKNAIISYIILELLHNGGQTMDFNARNKLINDLVNYGLRVKGSSKGSLFDELDIENPLNAYGFAFKQDLKKKIRDMVNSKRVLDGKSSVITYKADSPFSINKENMSFTHDYSSFEELSDHIRDNDTNLYFNFGSSGNPTIARFKINLGAGRHKKNKDELIATLLKLYSGEYQFCGSRIGIEKNKIILNLVLSIPKKVRALDENTVVGVNLGVAVPAMCALNNNEYERLAIGSADEFLRVRTKLQAQRRRLQKSLKDASGGHGRTKKLKALERVAKAESHFANTYCHMISKRIVDFALKNNAKYINLENLTGYDTNDFILRNWSYYKMQQYTTYKAEKYGIIVRKVNPCYNAQACSVCGNYAPGQRKSRAVFICANPACKSHKKNHGKLDAEFNNARNVAMSTLYMNDGQVTEKSFKEARDYFGIEEEIETI

SEQ ID NO: 188 - protein, 542 aa, unidentified
MAKKQNKNTMQINRKITLIPVASDNDGWKKKMNTYLEKFYFDKKIKAKERQIKNTSKPERKEEYKQQLTEYKKQQEQFLNGELEDYTKQMVMSYTYDLVRDCMESEARQKNFIMSYMFSEMIREKVCYLKNKKEKEKWVNDNINVAFRVKGSPKGSIFDDVEIYSPLRALSIQGYTQELKGRVKKFATDGGLDGKLAVDNYKLDSPFHMSKQGFDIVHEYEDLTELKKNIGKSSCEIYVNLGNGGVPTFARFQLDFGHKGNREELISTITKVFTGEYKVCGSSIQINKKNKIILNLGLEIPKRLTELDENTVVGVDLGLAVPAVCSLNNNQYKKEYIGDGEAFVKQRGKIQKEKQRLQKALKLSKGGHGRKRKMLALERFKEREANFVNTYCHRISKKVVEYALKNNAKYINIENLKGYDSSKFILRNWSFYQLQQDITYKAERYGIEVRKINPAFTSQVCSFCGYWESGQRINQKTFKCGNPNCKSHNLKFFNADYNAARNISMSTLFTSDNYKFGKESLQKAADYYGIDLEIDSEDQEIA

SEQ ID NO: 189 - protein, 492 aa, unidentified
MITVRKIKVRCEDKTFYDFMRKEQREQNKALNLSIGYIHTNSILKSVDSGAETLILNSIEKLNKKVDKLKKDLEKPKITDKKREQTEKAIQTNLKLIKDEQIKLEEGKQFRQGLDKQFSEIYINNNNLYHVLKSQTQVQYMRTLDLVTQKVKQDYSNNFVDIVTGKCSLMNYKSDFPLMIDKKCINIFKEEQNYKIRIMLGYELDIILGRRNNENVNELKSTLEKCISGEYKICQSSISINKNDVIFNLTLDIPNTTNYEPVHGRVLGVDVGVKYPVYMCVSDNTYKRKHIGSAADFLKVRQQFQERKRKLQASLDITKGGKGRKKKTQALERFKDKERNFAKTYNHQVSKKVVEFAKKNKCETIALEKITKEGIGDTILRNWSYYELQHMIEYKAKRECIKVEFVDPAYTSQTCSKCGHVSKDNRQTQEHFKCVNCGYELNADHNAAINIARRSIDYKPKEIIEKTIEKTIQPITTDLVEETGEFKQLTFI

SEQ ID NO: 190 - protein, 446 aa, unidentified
MQRSITLKIIRASDDKITWEELGYLLRGLSLKICRMSNFCMTHHLLHALKLETEMLNPKGNLYCYPHLAEEYPEVPAGIVCAAESRARKLFRRCGAKVLRSEMSLPSFRKDNSIPIPVAGYRILQDGDTNYAEIQLLSRQGAKTQKLPGRIRLTLADNWRDKAARPALQKLAAGKIRRGVASLYRAKNDWYLCIPYEVEAANTGGDFEPGLVMGVAFGVYNALVYGFNTLLKRGAISGEEILSHQAKVKARRKKIQEQYPWSGRKGQGREDTLKPLRHLHEAEKNYRELVNHRYAKWVVDIAVKNRCGEIHLDDGHAPPLGKNKILLSRWSLYDLKNKICRKAEEKGIRVTECSVPDLRTRCSHCGTEQVAGNHKRMFLCADCGYGSTEKNRGTGYISVDYNAARNLAMYDTGKNEPNMERDLIPKDDPAYDRNEGFREELSGSGQ

SEQ ID NO: 191 - protein, 488 aa, unidentified
MATEYTCNTRKIEVHLHRHGEDEEAKQRLIDDYRVWDTINDNLYKAANRIVSHCFFNDAFEYRLKIHSPRFQEIEKLLKYPKRNKLTDEDIKQLKAERKQLFADFKKQRHTFLRGGVAEGANPEQNSTYKVISNEFLEVIPSEILTNLNQNISSTYKNYSLDVERGIRTIPNYKRGIPVPFSIKQRGELMLKRRDDGSIYVRFPLGLEWDLSFGRDRSNNREIVERVLSGQYDVGNSSIQESKNRKRFLLLVVKIPKENHNLNPDRIVGVDLGINIPLYAALNDNEYGGMGIGSREQFLNMRMRMVAKKRELQRNLRQSTNGGHGRAQKLQALERFEGKERNWVHLQNHIFSKSIIEYAVKNNAGTIQMERLTGFGRDKNDEVDSDFKFILRYWSFFELQTMIEYKANAAGIEVRYVDPYHTSQTCSFCGHYEKGQRLNQSTFVCKNPDCEKGKGKKLSNGTYQGINADWNAARNIALSDKIVDRKKK

SEQ ID NO: 192 - protein, 456 aa, unidentified
MLITRKIELHISEADPELRKEHWKYLSFLNSEIYKAANLIVTNQLFNNFLENRVVDADGNVMDIGKRIRSLYRNKDKNADEIEKLKAAKLELRTEVKKFYLKSKQNTTYSITSHNFPEIDASIITTLNAQITGVLKKEWNEVERGSRSLRTYKKGMPIPFNLTSKSKKWFEKVDDEIFLTWFKGIKFKLFFGRDRSNNRAIVDRCLSGEYKYSDSSIQYKDRKIFLLLVVDIPESTKVLDENVSVGVDLGITVPAYCALSDGLKRLAIGSSEDLLRVRLQFQSRKRRLQRALKSSKGGQGRDKKLKALDNVADKEKRYVSTYNHMISSNIVKFAKDNNAGVIKLEMLEGFGEDEKNKFILRNWSYYQLQTMIQYKAKRENIKLVFIDPYHTSQTCSICNHYEAGQREKQSEFICKNPDCKNFDTPINADFNAALNIAVSKKVVTSKEDCEYFKKEN

SEQ ID NO: 193 - protein, 496 aa, unidentified
MILTRKIKLVIVSENQKEGYSLIRNEIREQYKALNLAYNHLYFEHNAIQKLKQNDEDYKQKRSKLQELINKKYEEHQKVKNLEKKEALREAYNKKKQELYNFEKEYNEKARQAYQQVVRFTQQTRVRNLINRECNLMSDTKDGITSKVTQDYKNDCKAGLLIGKRSLRNYKKDNPLLVRGRSLKFYKEDGDYFIKWNKGTIFKCILHIRKKNVAELQSVLENVLLGAYKICDSSIGFNNKDMILNLSLNIPDKETHDYIPGRVVGVDLGLKIPAYVSLSDKVYVRKGIGSIDDFLRVRTQMQKRRRRLQESLAAVKGGKGREKKLKALDHLKGKEANFAKTYNHFLSTQIVTFAVKNQAGQINMEFLEFDKMKNKSLLRNWSYYQLQMMVEYKAKREGIIIKYVDAYLTSQTCSKCDHYEEGQRETQERFKCKSCGYEVNADYNASRNIAKSTRYISDSTESEYHRKKQEALKEILGENDTINEQLSLFDKRDDIA

SEQ ID NO: 194 - protein, 415 aa, unidentified
MQRSLKLKIVRPYDESITWEEIGYLLRGISYQICKMSNYCMTHHLLRALGMETENLNPQGNLYCYPRLAKEYPDVPTGIICAAEGRARKLFKRNAPGILRSETALPSFRKESSIPIPVAGYSLAKIGPDIYVADVQLLSRKAAKTGKLPGRIQFVLANNWRDKKAGSVLHQLAEGTIKRGIASIFRNNRDWYIGIPYSVDPAPSDGELDPDLVMGVAFGTHSALAYAFNNLLKRGELGGEEVLSHREKFKARRQHIREQYNWSGRKGHGRENALKPIRELYEKERNYRNLTNERYAKWVVEIAKKNHCGKINLDAGGDRRNGKQNILLAYWPQSELHMKIKNKAEAYGIQVQECTATDIRRRCSRCGAVQETSGDKRWFICSHCGYGKEEKKTSSGFVSVDYNAARNVSMWEGTT

SEQ ID NO: 195 - protein, 461 aa, unidentified
MIAVRKLKVICKDKEFYDFFNMEQREQNKALNMAISLRHVNNTLKRIDSGAEVIIIKSIDKLNRKIETLEKELKNENITEAKKEKTLKDIETHKKIIKGEKKKLEEGRIYRKGLDKEFSQNYFDKTQLYHVLDGMTSIQHKRTIELVREKVKKDYENNFIDIVTGKQSLPNYKSDFPLMIDGSSIHIFEEDGIYKIKIMRGYELEVILGRRENENILELRKTLDRCSNGQYKVCQSEIQRDKNNNIIFNLTIDIPIENKKYTPVEGRVLGVDLGIKYPVYMCLNDDTYKRTSLGNINNFLRVREQMQERRRAFQKDLTLTKGGKGKSKKTQLLNKLRENEKNFCKTYNHTMSKRVVEFAKKHNCEYIHIENLTKDGFSNSILRNWSYYELQKFIKYKADREGIVVKYIDPAYTSQMCSKCGYTDKENRQTQAEFKCISCGFKLNADHNASINIARSNKFIK

SEQ ID NO: 196 - protein, 468 aa, unidentified
MPTITRKIELNLCTEGLADEEQKAQWNLLYHINDNLYKAANNISSKLYLDDHVSSMVMLKHAEYLTLVRALEKAQKQKTPDETVIEDLRRQVAAAEKDMTDQELAICNYATEMSTQSLSYRFATEIETNIFAQILDCLKQGVYATFNSDAKDVKRGERAIRNYKKGMPIPFPWNKSLKIEHKDGEFYLLWYNGLRFHLNFGKDRSNNRLIVQRCMKMDKDYEGDYKMCNSSIQFVKREGKPKFFLLLVVNIPQEHVELDKKIVVGVDLGINSPAYVATNVTMERQQIGRRETFLNGRMSFQRRYKSLQRLQTTAGGRGRKKKLEPLERLRNAEHNWVHTQNHLFSREVVQFAVKARAATIHMEDLSGFGKDNDGNADERKEFVLRNWSYYELQTMISYKAQKYGIKVEKIRPAYTSRTCSWCGHEGFRKGEIFICENPACEKCGEKENADYNAARNIANSKDIIKNHG

SEQ ID NO: 197 - protein, 462 aa, unidentified
MGNNRMTICRKIKLFPVGDKDEINRVYDFIRNGQYAQYQACNLLMGQLMSEYYKYNRDIKNEEFKARQKEIMTNSNIILKDIDFATGVDTPSAVTQKVKQDFSTALKNGLAKGERTVTNYKRTNPLITRGRNLTFYHEYETYHDFLDKINDSDLAVYVKWVNKIVFKVVFGNPHRSLELRSVIQNILEENYKVQGSSIEIDGKSIILNLSISIPKQLRELDENIVVGVDLGIAVPAMCALNNNLYERLAIGNADDFLRIRTKMQAQRKRLQKSLRNTSGGHGRAKKLKALERLQKAEVHFVETYCHMISKRVVDFALKHNAKYINIENLTGYDTSDFILRNWSYYKLQDYITYKAAKYGIEVRKINPCYTSQICSVCGNWEFGQRKSQSVFECANENCDSHKKYEKTGFNADFNAARNIAMSTLWMGSGQVTEKSKQEAREYYDISEKYEQSKNGSENNKVA

SEQ ID NO: 198 - protein, 423 aa, unidentified
MNIVKKIKLRIIDNDKELCKKQYLGFTEEQKKELIDKQYKFIRDSQYQQYLGFNRAMGFLMSGYYANNMDIKSDNFKEHQKKLTNSLYIFDDIKFGVGIDSKSLIVQRVKKDFSTALKNGLAKGERSVTNYKRTYPLLTRHRSIKFLYAENELDIYLDWVNKIRFRCELGNHKNSLELQHTLRKVITGEYKISDSSLEFNKKNELILNLNLNIPETKATFIKDRTLGVDLGMAIPAYVSLSDTPYIRKGFGSYEEFAKVRNQFKDRRKRLLKQLSLVAGGKGRAKKLHSMEFLKNKEKQFAKTYNHSLSKKIIDFALKNNCEYINLEDIKSTSLEDRVLGQWGYYQLQEQIEYKAKLVGIKVRKVKAAYTSQTCSECGNIDKENRKNQSTFKCTNEDCKLNKKGINADWNASINIARSKEFIK

SEQ ID NO: 199 - protein, 488 aa, unidentified
MATDYTVITRKIEVHLHRHGDSEEAIQRYKEEFRIWDEINDNLYKAANRIVSHCFFNDAYEYRLKIHSPRFQEIEKLLRYSKRNKLTDEDIKQLKNERKQLFTDFKKQRLAFLRGGATEGTNPEQNSTYKVVSNEFGDIIPSDILTCLNQNIASVYKAYSKEVEFGMRTIPNYKKGIPIPFTIRPNGVLNLQKREDGSIYIRMPKGLEWDLSFGRDRSNNCEIVERVLSGQYDVGNSSLQESKNKKRFLLLVVKIPKVNKALNQDRVVGVDLGINTPLYAALNDNEYGGMSIGSREQFLKMRMRMNAQKRELQHNLRHSTNGGHGRKQKLQALERLEGKERNWVHLQNHIFSKSIIEYALKNDAGVIQMERLTGFGRNKNDEVDEGYKFILRYWSFSELQNMIEYKANAAGIEVRYIDPYHTSQTCSFCGHYEKGQRISQSTFVCKNPECTKGKGKHKSDGSYEGINADWNAARNIALSNNIVGRKKK

SEQ ID NO: 200 - protein, 481 aa, unidentified
MATEYTCITRKIEVHLHRHGDSEEALQRYKEEFRIWDEINDNLYKAANRIISHCFFNDAYEYRLKLHSPRFQEIEKLLKYAKRNKLTDDDIKALKAERKELFAEFKRQRQSFLGGSEQNSTYKVVTDEFLEVIPSHVLTCLNQNISSTYREYALDVEHGRRTIPNFKKGIPVPFPIKATGELLLRKREDGSIYIRFPKGLEWDLNFGRDRSNNREIVERVLSGQYDVGNSSIQETKNRKRFLLLVVKIPKESRALNPDRVVGVDLGVAVPLYAALNDNPYGGMSIGSADQFLKVRMRMAAQKRELQRTLRNSTNGGHGRKHKLQALDRLEGKERNWVHLQNHIFSKELVEFALRNEAGAIQMERLTGFGHDRNDEVDEGFKFILRYWSFFELQTMIEYKAKAAGIEVRYVDPYHTSQTCSFCGHYEKGQRVNQATFICKNPDCTKGKGKERSDGTFEGINADWNAARNIALSDKIVERKKK

SEQ ID NO: 201 - protein, 464 aa, unidentified
MVEQGVTITLNKSDKNTYTIARKVRLAVDGDKEEVNRVYQFLRDGIYNQNKAYNIFISSVYSAIINGASTEELNEIYKRGSRKPKEDDETYSLYKFGEIEFPVGVGTTASLKQMVKNDLKKAKNDGLFKGKISLPNRKLNAPLRIESACFSFIHNYNSYQEFLDHLYTDDCEIIMKFVNNIRFKVNLGQACKTHELRSVFQNIFEENYKVCGSSIEIDGTKIILNLSLTIPKKKHELDESIVVGVDLGIAVPAVCSLNNNTYIKKFIGSRNDFLRERTKLKAQRRNVQKSLKFTSGGHGRKKKLRHLEVFTEHEKNWVKNYNHRVSKEIVDFALSNDAKYINIENLQGFDSSNFLLANWSYYQLQQQIEYKADMHGIIVRKVNPYHTSQRCSCCGFESPDNRPKDKKGQAYFKCLNCGTEMNADFNASQNIARSTDFSIGEVTLEEDKKKHNKSKKIPKEKQAA

SEQ ID NO: 202 - protein, 434 aa, unidentified
YISSHMFFNEAYIERLKAHSHRYREIKKTLKKIDEFTPEEAKALNDEKRVLEKQFVEERLRFLKGGDANGSGSVMSSTRQAVVEAFPEIPFKVLDCLNREMSKTFSQYKRDIENGKRTLSNYKKYYPVPFSMEQGKQLRKREDGSIYVMFPGKLEWDLYFGKDPSNNREIVERIFNGEYQACDSSIKEIKGRKKQILHLVVKIPKKTIKRDSNRVVGIDLGVNTPLYAALNDSERERFSIGSREAFLNVRMRFNAQRRELQRNLRHTTNGGHGRKQKLQALERLEGKERNWVHLQNHIFSKSLIEFAQKCEAGVIQMEELSGFGRDKFDSVDDGYKFILRYWSFFELQNMIEYKANAAGIEVRYVNPAYTSQTCSYCKHYEKGQRISQSTFVCKKCTEGKGKQRKDGSYEGINADWNAARNIAMSDKIVDRKKK

SEQ ID NO: 203 - protein, 466 aa, unidentified
MITTRKLKLTILGDEETRKLQYKLIRDEQYEQYKALNLCMSLLNTHNILSGYNTGAENKLNSQIDKLNKKLEKAQFEIKKNDIKQSKLDKLNSDIELYQNELNKLKEQFTQSSKYRSDIDLKFKEMYIDDLYTIVQNQVTFKNKDLMSLVTQRAKKDYSIALKNGMARGERSLTNYKRDFPLMTRGERWLKFKYDENSDDILIDWISGIKFKVILGYRKNENSIELRHTLHKVINKEYKICDSSIQFDRNNNLILNLTLDIPNNSKSEFIENRTLGVDLGIKYPAYICLSDDTFKRESIGCAEDFIRVREQIRNRRKRLQQQLKMVQGGKGREKKLKALDRISDKERNFVKTYNHMISKNIVNFAKKHKCQYINLEKLTKDGFPNMILSKWSYYELQQMIEYKAERENIKVRYIDPAYTSQTCSRCSHIEKDNRETQEKFKCLKCEFELNADHNAAINISRSVNFK

SEQ ID NO: 204 - protein, 461 aa, unidentified
MISTRKIKVRCDDNTFYTFFRQEQREQNKALNIGMGIIHADAILHNIDSGAEKKLKKSIEGLQGKIDKLNKYLEKENITDKKKKEVLKAIETNKKILDGEKKAFKESEEYRKGIDELFKSTYLKSNTLYHVLDSMVNIQYKRTLSLVTQRIKKDYSNDFVGVVTGQQSLRNYRNDNPLMISNQQLDLKYIEDTFYLDVMCGYRLEIILGRRDNQNVNELKSTLHRILSKEYKVCDSSMQFDKNNKDVILNLIIDIPNKSNMYEAIKERTLGIDLGVEVPIFMCLNDNTYIKKGIGDINNFLRVRQQIQIRRRKLQKDLTLTNGGKGRKKKLQLLEKLQENEKNFVKTYSHVLSKRIIEFAKKNKCEYINLEKLTKDGFDNIILRNWSYFDLQRMIEYKAKREGITVRYVNPSFTSQKCSKCGEIDKENRQTQADFKCTNCGFELNADHNAAINIARSIDFV

SEQ ID NO: 205 - protein, 417 aa, unidentified
MQRSIKLKILRPFDGNITWEALGYLLQGLSFKVCRISNFCLTHHLLRALKLETENLNPKGHLYCYPRLAEEYPEVPAGIICAAEGRARKVFNQKGKSIMRSEMALPTFRKNCSIPIPTAGYNLRWEGKDTCIAEVQLLSRQGAKTGKLPGRISLVLADNRRDKTAGAVMRKLAEGTLRRGAATLFREKKDWYISIPYETEAIHTEKDFVPGLVMGVAFGIRCTLAYGFNRLLKRGEIKGDEVLAHQEKYLARRKKIQEQYNWSGRRGHGREHALKPLRHLYEKERNYRSLVNARYAKWIVEIAEKNRCGAIHLDGENRLQRGKYPALLARWPLEELRQKIREKAELYGIKVSECTEAGIRERCSRCGAVQEDAADGYKFTCTACGYGAKGNNSSTGYISVDYNAARNLAVWEPETDS

SEQ ID NO: 206 - protein, 484 aa, unidentified
MATEYTCITRKIEVHLHKHGDSEEALQRLNEECRIWDEINNNLYKAANRIISHCFFNDTYEYRLKLQSPRLQEIEKLLSNPKRNKLSDEDIKQLKAERKQLFANFKKQRQVFLRGGVEEGANPEQNSTYRVVSNEFIDVIPSEVLTNLNQNISSTYREYSLDVERGSRTIPNYKKGIPVPFSIKRSGELMLKKREDGSIYVRFPKCLEWDLFFGRDRSNNREIVERVLNGQYDVGISTIQETKNKKRFLLLVVKIPKESKKLNPNRVVGVDLGINIPLYAALNDNEYGGLGIGSREQFLKVRMRMVAQKRALQRNLRHTTNGGHGRAQKLQALDQLEGKERNWVHLQNHIFSKSIIEYALKNGAGVIQMERLAGFGRDKNEEVENEFKFILRYWSFFELQTMIEYKANVAGIEVRYIDPYHTSQTCSFCGHYEKGQRINQSTFVCKNPDCVKGKGKQHADGSYDGINADWNAARNIALSTTVVD

SEQ ID NO: 207 - protein, 417 aa, unidentified
MQRSIKLKILRPFDGNITWEALGYLLQGLSFKVCRISNFCLTHHLLRALKLETENLNPKGHLYCYPRLAEEYPEVPAGIICAAEGRARKVFNQKGKGILRSEMALPTFRKNCSIPIPTAGYNLRWEGKDTCIAEVQLLSRQGAKTGKLPGRISLVLADNRRDKTAGAVMRKLAEGTLRRGAATLFREKKDWYISIPYETEAIHTEKDFVPGLVMGVAFGIRCTLAYGFNRLLKRGEIKGDEVLAHQEKYLARRKKIQEQYNWSGRRGHGREHALKPLRHLYEKERNYRSLVNARYAKWIVEIAEKNRCGAIHLDGENRLQRGKYPALLARWPLEELRQKIREKAELYGIKVSECTEAGIRERCSRCGAVQEDAADGYKFTCTACGYGAKGNNSSTGYISVDYNAARNLAVWRQETNS

SEQ ID NO: 208 - protein, 461 aa, unidentified
MGNDRMTICRKIKLFPVGDKEEINRVYDFIRNGQYAQYQACNLLMGQLMSEYYKYNRDIKNKEFKARQKEIMTNSNIILKDIDFVTGVDTPSAVTQKVKQDFSTALKNGLAKGERTVTNYKRTNPLITRGRNLTFYHEYETYQNFLDKINDSDLAVYIKWVNKILFKVVFGNPHRSLELRSVVQNILEENYKVQGSSIEIDGKSIILNLSISIPKQLRELDENIVVGVDLGIAVPAMCALNNNIYERLAIGNADDFLRIRTKMQAQRKRLQKSLRNTSGGHGRAKKLKALERLQKAEVHFVETYCHMISKRVVDFALKHNAKYINIENLTGYDTSDFILRNWSYYKLQDYITYKAAKYGIEVRKINPCYTSQICSVCGSWEFGQRKSQSVFECANENCDSHTKYERGFNADFNAARNIAKSTLWMESGQVTEKSKQEAREYYGISEKYEQSKNEVENNKVA

SEQ ID NO: 209 - protein, 419 aa, unidentified
MQRSIILKIIRPYDESIKWDDVGYLWRGLSFKVCKISNYCMTHHLLRAMNLETENLNPQGRLYCYPHLAKEYPEIPAGIICAAEGRARKVFKQNAKGILYSETSLPSFRKDCSIPIPVSGYSLLKAGADTYVASIQFLSRQAAKTQKLPGRIQLVLASNWRDKSAGRILQQLAEGTLKRGIASLFRKKRDWYFSIPYEVEPSGADDSFEPELAMGVVFGFQCALAYGFNRLLKRGMLGGDELLAHREKMLARKKQILTQYRWSGRKGHGRESALKPLQALYEKERNYRNLTNERYAKWIAEIAKKNRCGKIYLDTGGSSGHGTQNILLAYWPKEALRKKIMNKAEAYGIEVVECTDAEIRNRCSRCGTLQEPLENKKWFVCNHCGYGKDDKKTGDGFISVDYNAARNLAVYDTGSKESV

SEQ ID NO: 210 - protein, 461 aa, unidentified
MISKRKIKVRCDDNTFYTFFRQEQREQNKALNIGIGIIHSNAILHNIDSGAEKKLKKSIESLQGKIDKFNKHLEKEKLTDKKKEEVLKAIETTKKILDGEKKAFKKSEEYRKGIDELFKSTYLKSNTLDHVLDSMVNIQYKRTLSLVTQRIKKDYSNDFVGIITGQKSLRNYRNDNPLMISNQQLDFKYIEDTFYLDVMCGYRLEIILGRRDNQNVNELKSTLHRILSKEYKVCDSSMQFDKNNRDVILNLGIDIPNKSNMYEAIKGRTLGIDLGVEVPIFMCLNDNTYIKKGIGDINNFLRVRQQIQVRRRKLQKDLTLTNGGKGRKKKLQLLDKLQENEKNFVKTYSHALSKRIIEFAKKNKCEYINLEKLTKDGFDNIILRNWSYFDLQRMIENKAKREGITVRYVNPSFTSQKCSKCGEIDKKNRQTQADFKCINCGFELNADHNAAINIARSLDFV

SEQ ID NO: 211 - protein, 401 aa, unidentified
ITWEALGYLLQGLSFKVCRISNFCLTHHLLRALKLETENLNPKGHLYCYPRLAEEYPEVPAGIICAAEGRARKVFNQKGKGILRSEMALPTFRKNCSIPIPTAGYNLRWEGKDTCIAEVQLLSRQGAKTGKLPGRISLVLADNRRDKTAGAVMRKLAEGTLRRGAATLFREKKDWYISIPYETEAIHTEKDFVPGLVMGVAFGIRCTLAYGFNRLLKRGEIKGDEVLAHQEKYLARRKKIQEQYNWSGRRGHGREHALKPLRHLYEKERNYRSLVNARYAKWIVEIAEKNRCGAIHLDGENRLQRGKYPALLARWPLEELRQKIREKAELYGIKVSECTEAGIRERCSRCGAVQEDAADGYKFTCTACGYGAKGNNSSTGYISVDYNAARNLAVWRQETNS

SEQ ID NO: 212 - protein, 419 aa, unidentified
MQRSIILKIIRPYDESIKWDDVGYLWRGLSFKVCKISNYCMTHHLLRAMNLETENLNPQGRLYCYPHLAKEYPEIPAGIICAAEGRARKVFQQNAKGILYSETSLPSFRKDCSIPIPVSGYSLLKAGADTYVASIQFLSRQAAKTQKLPGRIQLVLASNWRDKSAGRILQQLAEGTLKRGIASLFRKKRDWYFSIPYEVEPSGADDSFEPELAMGVVFGFQCALAYGFNRLLKRGMLGGDELLAHREKMLARKKQILTQYRWSGRKGHGRESALKPLQALYEKERNYRNLTNERYAKWIAEIAKKNRCGKIYLDTGGSSGHGTQNILLAYWPKEALRKKIMNKAEAYGIEVVECTDAEIRNRCSRCGTLQEPLENKKWFVCNHCGYGKDDRKTGNGFISVDYNAARNLAVYDTGSKESV

SEQ ID NO: 213 - protein, 481 aa, unidentified
MATEYTCITRKIEVHLHRHGDSEEAAQRLKEEYRIWDEINDNLYKAANRIISHCFFNDAYEYRLKLHSPRFQEIEKLLKYAKRNKLTDDDIKALKAERKELFAEFKRQRQSFLGGSEQNSTYKVVTDEFLEVIPSDVLSCLNQNISSTYREYALDVEHGRRTIPNFKKGIPVPFAIKVHRELALRKREDGSIYIRFPKGLEWDLNFGRDRSNNREIVERVLSGQYDVGVSSIQEAKNGKRFLLLVVKIPKESRALNPDRVVGVDLGVNIPLYAALNDNTYGGLSIGSRDQFLNVRMRMAAQKRELQRNLRVATNGGHGRKQKLQALDRLEGKERRWVHLQNHIFSKSIIEYALRNEAGAIQMERLTGFGHDRNDEVDEGFKFILRYWSFFELQTMIEYKAKAAGIEVRYVDPYHTSQTCSFCGHYEKGQRVNQATFICKNPDCTKGKGKERSDGTFEGINADWNAARNIALSDKIVERKKK

SEQ ID NO: 214 - protein, 468 aa, unidentified
MPIITRKIELKLCTEGLSEETIKSQRMLLYHINDNLYRVANNISSKLYLDEHVSSLVRMKNGDYLSLEKQLVKAKKQKNPDTKTITELEERILTVKREMNSQEIAISKYATEMSTKTLAYNFAKELGSDIFGQILACLQQNVHSTFSEDAQEVRRGERAIRNYKKGMPIPFPWNRSIRIEALDGEFYLRWYNGIRFHLFFGKDRSNNRQIVKRALCLDPDYSGENYKLCNSSIQLVKREHERKTFLLLVVDIPKEVRKLNKDIVVGVDLGINVPAYVATNSTEERKEIGDREHFLNERMSFQRRYKSLQRLKCTAGGKGRKKKLEPLERLREAEHKWVHTQNHRFSREVVDFALHAEAATIHMENLSGFGKDQEGNADEKKEFVLRNWSYYELQNMIAYKAAKYGIKVEYVKPAYTSKTCSWCGKEGFRQSTIFICENHECKMCGIKVHADYNAARNIANSNNIIKNE

SEQ ID NO: 215 - protein, 481 aa, unidentified
MATEYTCITRKIEVHLHRHGDSEEAAQRLKEEYRIWDEINDNLYKAANRIISHCFFNDAYEYRLKLHSPRFQEIEKLLKYAKRNKLTDDDIKALKAERKELFAEFKRQRQSFLGGSEQNSTYKVVTDEFLEVIPSDVLTCLNQNISSTYREYALDVESGRRTIPNFKKGIPVPFAIKVHRELALRKREDGSIYIRFPKGLEWDLNFGRDRSNNREIVERVLSGQYDVGVSSIQEAKNGKRFLLLVVKIPKESRALNPDRVVGVDLGVNIPLYAALNDNTYGGLSIGSRDQFLKVRMRMAAQKRELQRNLRVATNGGHGRKQKLQALDRLEGKERRWVHLQNHIFSKSIIEYALRNEAGAIQMERLTGFGHDRNDEVDEGFKFILRYWSFFELQTMIEYKAKAAGIEVRYVDPYHTSQTCSFCGHYEKGQRVNQATFICKNPDCTKGKGKERSDGTFEGINADWNAARNIALSDKIVERKKK

SEQ ID NO: 216 - protein, 417 aa, unidentified
MQRSITLKIIRPEDETISWEELGYLLRGLSFKVCRMCNFCMTHQLLHALKLETELLNPQGNLYCYPRLAEEYPDVPTGIICAAETRARKLFRRSAEAVLHSETSLPRFRKDSSIPVPVAGYKILQDADHNVYADVQLLSRQGAKTQKLPGRIRLVLADNWRDQSAKAALQQIVAGKVKRGVASLFRVKNDWYFQIPYVTEAVNTEEIFQPDLVMGVAFGLQDALVYAFSTSMKRGAVSGEEVLAHQEKYVARRKKIQEQYNWSGRKGHGREDALKPLRHLYETERNYRNLVNSRYAKWVVDIAAKNRCGVIHLDSANYVSSGKNILLSRWPLYDLKEKIRRKAEEKGIQVTECSIPNLRTRCSRCGKEQEPEGEKRTFVCKDCGYGKADKNRRGGFITVDYNAARNLAVYESEDAKL

SEQ ID NO: 217 - protein, 488 aa, unidentified
MATEYITITRKIEVYLHRHGDSDEAKQRYQQEWQIWHDINDNLYKAANRIVSHCFFNDAYEFRLRLHSPRFAEIEKALKHSKKNKLSDDEVKALKAERQELYKSFKQQRLAFLRGGATEGANPEQNSTYKVISDEFGDIIPSDILTCLNQNIASVYKAYSKEVQFGDRTIPNYKKGIPAPFSIKAGGALLLKKREDGSIYVRMPKGLEWDLVFGRDRSNNREIVERILSGQYDAGNSSIQQAKNGKCFLLLIVKIPKSNIALNKDRVVGVDLGINIPLYVALNDNEYGGMGIGSREQFLKMRMRMSAQKRELQRNLRHSTNGGHGRKQKLQALERLEGKERNWVHLQNHIFSKSVIEFAQKHNAGVIQMERLKGYGKDANGEVREEAKFLTRYWSYFKLQTMIEYKANAAGIEIRYIDPYHTSQTCSFCGHYEKGQRISQSTFICQNPECKQGKGKQKSDGTFEGINADWNAARNIALSEQYVDKKKK

SEQ ID NO: 218 - protein, 481 aa, unidentified
MATEYTCITRKIEVHLHRHGDSDEALQRYKEEYRIWDEINDNLYKAANRIISHCFFNDAYEYRLKLHSPRFQEIEKLLKYAKRNKLTDDDIKALKAERKELFAEFKRQRQSFLGGSEQNSTDRVVSHEFLDVIPSDILTCLNQNISSTYREYALDVEHGRRTIPNFKKGIPVPFPIKATGELLLRKREDGSIYIRFPKGLEWDLNFGRDRSNNREIVERVLSGQYDVGNSSIQETKNRKRFLLLVVKIPKESRALNPDRVVGVDLGVNIPLYAALNDNTYGGLSIGSRDQFLKVRMRMAAQKRELQRNLRVATNGGHGRKQKLQALDRLEGKERNWVHLQNHIFSKSIIEYALRNEAGAIQMERLTGFGHDRNDEVDEGFKFILRYWSFFELQTMIEYKAKAAGIEVRYVNPYHTSQTCSFCGHYEKGQRVNQATFICKNPDCTKGKGKERSDGTFEGINADWNAARNIALSDKIVERKKK

SEQ ID NO: 219 - protein, 481 aa, unidentified
MATEYTCITRKIEVHLHRHGDSEEAAQRLKEEYRIWDEINDNLYKAANRIISHCFFNDAYEYRLKLHSPRFQEIEKLLKYAKRNKLTDDDIKALKAERKELFAEFKRQRQSFLGGSEQNSTDRVVSHEFLDVIPSEVLTCLNQNISSTYREYALDVEHGRRTIPNFKKGIPAPFSIKANRELALRKREDGSIYIRFPKGLEWDLNFGRDRSNNREIVERVLSGQYDVGVSSIQEAKNGKRFLLLVVKIPKESRELNPDRVVGVDLGVAVPLYAALNDNQYGGMSIGSADQFIKVRMRMAAQKREMQRTLRNSTNGGHGRKQKLQALDRLEGKERRWVHLQNHIFSKELVEYALRNEAGAIQMERLTGFGHDRNDEVDEGFKFILRYWSFFELQTMIEYKAKAAGIEVRYVDPYHTSQTCSFCGHYEKGQRVTQSKFVCKNPDCTKGKGKERSDGTFEGINADWNAARNIALSDKIVERKKK

SEQ ID NO: 220 - protein, 468 aa, unidentified
MPTITRKIELTLCTDGLSDEERKAQWGLLYHINDNLYKAANNISSKLYLDEHVSSMVRLKHAEYLSLQKELAKAERQKMPDVDVIEELRERLSAAEQEMSDQELAICKYATEMSTNTLAYRFATEIETNIFGQILARLENNAQAVFLTDAPDVKRGERAIRNYKKGMPIPFPWNNSIKIECEGGEFYLRWYSGLRFHFNFGKDRSGNRLIVQRCLKLDKEYDGEYKLCNSSIQMVKRDGSTKFFLLMVVNIPQEYVELNKHIVVGVDLGINVPAYVATNITPERKAIGDREHFLNTRMAFQRRYKSLQRLKTTAGGKGRTKKLEPLERLRQAEHNWVHTQNHLFSREVVNFALQTHAATIHLEDLSGFGKDSDGNADERKEFVLRNWSYYELQNMITYKAAKYGIRVEKIRPAFTSRTCSCCGHEGFREGVTFICENPECQQFGEKVHADYNAARNIANSKDIIKKNE

SEQ ID NO: 221 - protein, 464 aa, unidentified
MATEFTCITRKIEVHLHRHGDSEEAIARYKNEWDMWDEINNNLYKAANRIVSHCFFNDAYEYRLKMHSPRFQEIEKLLRYTKRNNLSDNDIKQLKEERKNLFADFKKQRLAFLQGNSGIGSEQNSTYKVISNEFLDTIPSQILTNLNQNISSTYREYTQDIERGIRTIPNFKKGIPVPFSIKKGGDLMLKKRNDGSIYIRFPKGLEWDLNFGRDRSNNREIVERILNGQYDVGNSSIQETKNKKRFVLLVVKIPKENQKLDTNRIVGIDLGINTPLYAALNDNEYGGFSIGSRDQFLKMRMRMAAQKRELQRNLRHTTNGGHGRNQKLQAFKRLQDKEKNWVRLQNHIFSKSIIEYALKNKAGVIQMERLTGFGRDKNDEVNEDYKFILRYWSFFELQTMIEYKANAAGIEVRYIDPYHTSQTCSFCGHYEKGQRINQQTFICKNPDCTKGKGKQEKDGTYKGI

SEQ ID NO: 222 - protein, 469 aa, unidentified
MPTITRKIELTLCTEGLSDEQRKAQWGLLFHINDNLYKAANNVSSKLYLDEHVQSMVRMKHDEYLGLLKELARAEKQKMPDAAVIAELREKIAAAEKEMTDQELAICKYATEMPTQSLSYRLVTEIETNIFAQILDCLKQNVFATFNSDARDVKRGERAIRNYKKGMPIPFPWNKAIKIESQGDDFFLRWYSGIRFKFVFGKDRSNNRAIVMRCLKLDEDYDGEYKLCNSSIQMVKRDGGMKLFLLLVVSIPQEHVELNKKIAVGVDLGVNVPAYVATNITEERKAIGDREHFLNTRMQFQRRYKSLQRLKTTAGGKGRTKKLEPLERLRDAERNWVHTQNHLFSREVVNFAVQTHAATIHLEDLSGFGKDYDGNADERKEFVLRNWSYYELQNMITYKAAKYGIHVVKVRPAFTSRTCSCCGQQGFREGVTFICENPECKQYGEKVHADYNAARNIANSNDIIQDNHE

SEQ ID NO: 223 - protein, 487 aa, unidentified
MITSRKIKLSIVSNNSTEAYNFIRKEMKVQNKALNVAMNHLYFNSIARQKILLADKAYQQKLKAAINSQEKSFNTLKELEQKQCIEEDSEKKQALKERLIKTKNAYEKAKEKVSNLRKSRNKDSFQEYKNIIGQVEQTHLRDIISSQFNLHSDTKDRLTMIAKQDFENDIAEVLSGDRSLRTYKKNNPLYIRGRNTVLYKEGNEFFIKWIKGIVFKCILGVKNQNKTELYKTLECVLAEIHKICDSSMNFNQQDKLILNLTLDMPDKSEHRKVPERIAGVDLGLKIPAYFAVNDVPYIRKSIGKIEDFLKVRISIQSQKRSLQRALQSSKGGKGRKKKLKTLDQFKEKEKNYITTYNHFISKKIISLAVQYGVEQINLELLTLKETQKKLLLRNWSYYQLQQFIEYKANREGISIKYVDPFHTSQTCSKCGHYEDGQREKQDTFCCKSCGFKDNADYNAARNIAASTRYITNKKESEYYQQNNNEIA

SEQ ID NO: 224 - protein, 408 aa, unidentified
MVKLKHNEYASLEKKLQKAQKEKTSDDNLIADLEEKLAAAKREMTDQARAICKYATEMSTETFAYKLATEIETNVFGQILSYLKKAVQSNFSNDAQAVRRGERSIRNYKKGMPIPFPLKDRIKIKDNDFYLHWYNNIRFKLHFGKDRSNNRQIVNRCFNSVNDYKLGSSASIQIVKRNGSPKFFLLLVINIPKEEVELNKKIVVGVDLGINVPAYVATNMTEERKAIGDREHFLNNRMAFQRRFKSLQRLRGTAGGKGRTKKLEPLENLRKTERNWVHTQNHLFSREVVKFAVQTHAATIHMEDLSNFGKDKDGNADEHKEFVLRNWSYYELQEMIRYKAKKYGIEVRNVRPAYTSQTCSWCGEQGFRQGATFICKNPECKQYSEKIHADYNAARNIAKSKDIIKKNE

SEQ ID NO: 225 - protein, 448 aa, unidentified
LYKAANNISSKLYLDEHVSSLVRLKHKEYKDLMRELAKAKKQKNLDEASIIEMEGRLRSYEEEMTDQELAICKYADEMSSLSLAYGFATELELDIYAQILTQIQSKVHQDFQNDQKDVREGKRSIRTYKKGMPIPFPWNNSIRIEISKKNKVEEQEKKTRRDSEDYDFYLNWYNGLRFRLHFGKDRSNNYQIIKRCFKLDEYCHDNYQLKASSIQLVIKNKKPELYLLLVVDIPQEKYSLNNKVVVGVDLGINTPAYVATNVTEDRKAISDREHFLNARMAIKRRYRSLQRLKGTAEGRGRKKKLEPLERLREAESNWVHTQNHLFSRDIIKFALRVKAATIQMEKLEGFGKDEDGNVEEDKKFLLGEWSYYELQNMVKYKAAKVGIKVHFVKPAYTSQTCSWCGERGIRDGTAFYCQNPNCKQHGKKDINADYNAARNIAKSTEIVK

SEQ ID NO: 226 - protein, 470 aa, unidentified
MRIIRPYYGDEIEKFITAGKEKSKADGADGALTKIFWDRLKASHPEIVSAGEFYGLLCAMRMEAVVYYNRAISKLYQSLIVDVDGAQVSTAKALSAGPYHEFRERFTSYISLGLRQKLQSNFRRKELLRCQLALPTAKSDRFPIPISKQVDKQGKGGFKVSELQNGDFIIELPLMAYHKAKGKTEREYVELDAGPAILNIPVILSTQRRRANKTWFKDEGTDAEIRRVMSGEYKVSWLEILQRNRFGKAHGDWYVNFTIKYQPKDCGLDPKVKGGIDIGLSSPLVCAVTNSLDRLTIRDNDLVAFNKRALARRRTLLRRNRYKRAGHGSANKLEPITALTAKNELYRKAIMRRWAREAADFFRHNKAAAVNMEDLTGIKDREDYFSQMLRSYWNYSQMQTVLENKLKEYGIAVNYINPKDTSKTCHSCGHVNQHFDFSYRSANKFPMFKCVKCGVECGADYNAARNIAVA

SEQ ID NO: 227 - protein, 484 aa, unidentified
MIIARKIRLTVISDNRDEAYSFLRNEMYYYYKALNFAMNHVYFNYVAKEKIKMADDKYIEREQRYINAINNTHATLKKVRTESQKDLALKRIELNEQNLKKLRQSTSKEAREMLSQAISSAERTNTSDAVQKAFPMLTRDSIDFAASKATTEFNNDLKLGLLSGERVLRTYKKTNPLQIRGRLLKFFKEDGDYCIKFAKGIIFKCVLGIKRKNNTELAHTLEKVIDGTYKVCDSSFEYNDSKLILNLALQIDITARQQQPKVQGRVVGVDLGLKIPAFCALNDSIYIRERIGDIEDFLKVRTQLQARRKRLQRALKSAKGGKGQGKKLKALERFREHEKNFVKTYNHYLSKKIVHFSVLYGAEQINLELLQMAESQNKSILRNWSYYQLQQMIEYKAAKEGIKIKYVDPYRTSQTCSKCGNYENGQRKTQATFECKSCKFKENADFNAARNIAKSTAYITDKFQSEYYKNYMDCKLGARASGFH

SEQ ID NO: 228 - protein, 461 aa, unidentified
MISTRKIKVRCDDNTFYTFFRQEQREQNKALNIGIGIIHSNAILHNIDSGAEKKLKKSIEGLQGKIDKFNKHLEKEKLTDKKKEEVLKAIETTKKILDGEKKAFKKSEEYRKGIDELFKSTYLKSNTLDHVLDSMVNIQYKRTLSLVTQRIKKDYSNDFVEIITGQKSLRNYRNDNPLMISNQQLDFKYIEDTFYLDVMCGYRLEIILGRRDNQNVNELKSTLHRILSKEYKVCDSSMQFDKNNKDVILNLVIDIPNKSNMYEAIKERTLGIDLGMEVPIFMCLNDNTYIKKGIGDINNFLRVRQQIQVRRRKLQKDLTLTKGGKGRKKKLQLLDKLQENEKNFVKTYSHALSKRIIEFAKKNKCEYINLEKLTKDGFDNIILRNWSYFDLQRMIENKAKREGIVVRYVNPSFTSQKCSKCGEIDKKNRQTQADFKCINCGFELNADHNAAINIARSIDFV

SEQ ID NO: 229 - protein, 481 aa, unidentified
MIITRKIAITIVSEEAQESYNYLRQQMYYYYKALNFGMNHIYFNYVAKEKIKLADSAYKEREEKYINAIHIAKEKLQKDLSVSQRAQAEKSLEVNTNNLDKLRKAISKDAKETFQKVMGAVERTNVTDAIKKEFPILQRDSIDFAASKVASDFNNDLKLGLMTGSRTLRIYKRNQAYPFRSRRLKLYKENGDFFIKSSKSLLFKCLLGVKRQNSKELIQILEKILDNQYKICDSSLEFNKKKLILNLCIEVDENTHSENMKVPGVVKGRIVGVDLGIQIPAYCTLNDSPFKKKAIGSVDDLLRIRTQMQARRRRLSKNLISARGGKGRGKKLKALDRFEEYERNYVTTYNHFISKQIIQFTLQNQAEQINLELLQMEHTKSKSILRNWSYYQLQQMIEYKAKREGIVIKYVDPYHTSQVCSKCGHFEENQRMDQNTFRCKKCKYRTNADYNAAKNIANSTRYISSIQESEYYNIKNRNIVK

SEQ ID NO: 230 - protein, 409 aa, unidentified
MELKRTARVKLAIPDDRRDDLKRTMLTFREVAQRFADRGWERDEDGYVITSRTRLQSLVYKQVREDTGLHSDLCIGAVNLAADSLRSAVERMKAGKNVGKPTFTVPTATYNTGAVSYFTDGDGTGYCTLAAYGGRVRAEFVYPPDEDCPQRQYLGGDEWEPKGATLHYERDDGEYYLHVTVERDEPETELGEAENGTVLGVDLGVENIAVTSAGAFYSGGLFNHRRDEYERIRGSLQQTGTESAHRTIEKMGDRERRWNTDVLHRISKAIVQEAITHDCSHIAFEDLTDIRDRMPGAKKFHGWAFRQLYEYVEYKAAEFGIATTQVDPAYTSQRCSKCGTTLRENRTSQAAFCCQKCGYEVHADYNAAKNVATKLLRSGQKSPAGGATNQLALKSGTLNGNGDFTPASS

SEQ ID NO: 231 - protein, 462 aa, unidentified
MKEQLNKTLTFGLGKPLGWDIRGKAFIDPTEDQRREIYISLRATSRISAQMVNMLNAREYVRRIMKIPEAFVNEFKGSYIPIKQELKTLGLEEVDDISGATLSQTWALGVKPDFAGEHGKRLLMKGDRQLPTHRIDGTHPIYGRADGTKIILHEDRYFLIVQLFSSKWANKNEFPSGWIAFPVKIKPRDKTLAGQFKRIIDGEWKLKNSHILRNPRKRGNTWLGQVVVSYTPDPFKDIDPKIIMGIDLGVSVPACLHIRENGKAKKWAMQVGRGRDMLNTRGIIRSEIVRIIRSLRSKDSPLDNESKRAAKAKLKNLRKREKRVMKTASQKIAASIADVARRNGAGTWKMELLSENIKDEDPWLRRNWAPRMVVDAVRWQAEQVGAKLEFVDPAYTSQRCSKCGHISRENRPKGKKGAAHFECVRCGYKDHADKNAARNISTPGIVDLIKEQISKSPNGEER

SEQ ID NO: 232 - protein, 405 aa, unidentified
MCRVLRRTVCLKLDVPDDRRDDLHETTDRFRQAAQIAVDRAFERNDDGYVITHKTKLHHLTYEQAREATDGLNANLVQAARNLAGDAAKGVVSRWENGKRASKPEFTAPTVVYNKKALTYREDGVSLATVNGRVECDFVLPPEGENPQTEYLRGDEWELRESTLHYRSESGEYYLHTTVAKDEDVSEEAENGTVLGVDVGVENIAATSTGRFWSSGLLNHRREQYESVRAGLQQTGTESAHRTIQQLGEREQRWVDDLLHRISKDIVSEAVEHDCSRIAFEDLTDIRERMPGAKKFHAWAFRRLFDLVSYKADERGIRTVQVDPAYTSQRCSKCGHTARNNRPSQAEFRCGRCGYENHADYNAAKNVAMKHVRAGQKSPRGRANRHLALKTGTLNGNGEFSPADA

SEQ ID NO: 233 - protein, 461 aa, unidentified
MVITRKIELHLVHTGLSDEEYDQQWKFLHSINDNLYRAANRLVNQLYLNDEIDILLRYNNKEYMELRKQLAKKNLDKPVRAELKEKEKLVLEEIKAHRSAIFQRPYASVAYSMVTSQNEANIMTKILDVLKQDVLSHYSTNAKEVARGERSISNYRYGMPIPFAFDRTEKASICIYEENKKYFLKWYNNLRFELSFGRDRSNNQLVVQRCLGISNDGAKYKACNSSIQMVRKNGSTRLFLLLCVDVPKEINKHIKGKVVGIDLGLNVPIYAAVNDGPERKSIGSREAFLDQRASFQRRFRSLQKLQMTKGGHGRLHKLEPLERVREAERNWVKNQNHLFSKEVVEFAKKVEAEIIQMERLKNFGRDDNEEIKEDKKYVVRNWSYFELQSMIEYKAKRAGIIVQYVNPAYTSQTCSECGQKGIRDNIHFKCLNPECNCFGKDIHADYNGARNIAKSKEIVKD

SEQ ID NO: 234 - protein, 398 aa, unidentified
MKIKRTIKLVVKPSEKEKHILFKTFEEYKFAYNFVAEIGWKSNIHNSVKLHNLTYIVVREKTSLPSQLVISARNVASESLKSAFSRKKKGLAVSCPYSKNPAIRYDKRSYSVWFDREEISIATIEGRLKLKIKIPEYFRQYFSWDIRSASLKYNKRLKKFFFNIVVEKEIEEIPENDTVVGVDLGLSKLATLSTADGKINKFFDGGHIRAVSERYFAIRKKLQSKGTPSAKRHLRKLSQKEKRFRTAINHKIAKEIVNLIPAGGTIVLEELKGIRERIRVYKRERRWVHSWNFAQLKQFIEYKAKSKGIKVVYINPKYTSQRCSKCGYVSKSNRKDQSHFKCSYCGYTVNADLNASRNIAINYLVSQKERLGHRVASLPVWAVVNQPNVRRLAVSSHS

SEQ ID NO: 235 - protein, 403 aa, unidentified
MQRTIRIQLKPDIETDSVLSQTIEQYTWSFNAVCKHGWKNDLANGVELHKATYYDHRAITGLPSQLVCAARVKATEALKSAKSLKKKGKTVSCPISKRCPIRYDARSYTAWFDRSELSILSINGRVKLSFEIAEYYRQYLAWKNTSADLLQDRKGCWWLHVVMEIETPQASVTDEVVGVDLGIASPAVDSRGSKYGSGHWKKIEDKTFELHRRLQSKGTKSAKGHLKKLSGRQRRFRKDCDHVLSKRLARSVESGATLVFEDLTNIRGRAKMRKAQRRRLHGWSFAQFQAFVTYKAEARGVNVGFVDPRYTSQKCSQCGHIERGNRPSQAEFRCKKCGYERHADYNAAINIRAEGIRMLKAEGSAVSAVGGEIRPKLGRKSKLRHSPVSTEADTVLGTPSQCG

SEQ ID NO: 236 - protein, 455 aa, unidentified
MPTIRRKIELLLDRTGLSKEEVDARWHTLHQINNNLYRTANNLINKLYLTDEIDDILRLNNQEYIDLKKQLGKKGLDETTKAELEERMHKIYATMNQHRSEILQRPRQSFAYSAVTGGDDTEIFNAKILDTLKQNVLAHYNADMKEVRRGEKSISNYKKGMPIPFSFDKSVRLYERNGQFFLKWYRDIQFILFFGHDASNNQLIVERCLGISEDGIAYKMCSSSLQMKGKKIFLLLVVDVPKEPFEQQKGMVVGIDLGLNVPIYATTNLTPERRAIGNRESFLNQRIAFQKRYKALQRLQLTKGGRGRSHKLEPLERLRETERNWVRTQNHLFSKEVIEFAKQVGASTINMERLASFGKNMSGEVYEDKKFVLRNWSYYELQNLIEYKAKRANIKVRYVNPAFTSQTCSECGQTGERDSIHFKCTNPECKNFGKSIHADYNGAKNIAKSTNIIKE

SEQ ID NO: 237 - protein, 472 aa, unidentified
MVITRKIELWIAEEDKTKRNETWDFLRMLDKEIFRAANVVVNNQYFNDFYEERIIQQDEKIGDVSKKIRLLYSKLRKANDEEQQSLQNEIEILKEQKKESQTRVRSEFYKTSKQNTTYQILTKQFPEIPSDILTCLNNQIYSVIGKEKKEVLQGKRSIRSYRKGMPIPFRFAQHPRLEFFENEYYLRWLNNISFVLRFGRDKSNNRAILEKIFSKEYKLCDSSIQIDDRKIFLLLVVDIPKSEHNLNKELSVGVDLGLNVPAYCALSEGFARLAIGHKDDFVRVRQQMQRRRKALQKSLVLTSGGKGRTRKLKALDALGEKERHFVRTYNHTVAKRIVEFAEKYNAGVITMELLEGYGKDENGKSRKGDFVLRNWSYFELQTLLKDKAGRKGMDVVFIDPYHTSQTCALCGHYEIGQRETQANFICKNPVCKNFDEKVNADYNAALNIARSKKFVAKKEECEYFKLHKETRS

SEQ ID NO: 238 - protein, 412 aa, unidentified
MKRTVKIKLSMNERKALEETIKQFKRACQMTVEEGWNENGLNNYKKYKLQKRVYDEIRETTDLQANLVVRAIARGAQAVKGCTQLFENGFKASKPNFTSDSIAYDKRTLSVYPDEKRCTISTVNGRIDANFVLPEEKNDYYKEYLDGSWEITQSTIEKHEYEKENSFYLHLGLEKEDEEIEHEDPTVMGVDLGMNNLAVTSTGKFFKGNQLDHNRKRFEEMRGKLQQKGTRSAHLTIQRMSERENRYACDTLHCISKELVEEAERNDVDIITFENLKYIRERMPKNKWYHVWAFNKLYQYVEYKAKERGIQVKQIDPRNTSRRCSKCGHTEKNNRNGNKFKCKQCGYELDADYNAAKNIGIKLLHRRQKSSAGAGNGQLALKSGTLKLNGEYSPTFLSARLCLRYFGIQKEF

SEQ ID NO: 239 - protein, 576 aa, unidentified
MNTIKNTYIRTLKFNLNLTPKFETEEDNKKYINDVYSYLRDAIWAQNRAMNIVLDRTKEAYTLGRGMNRVKEIYYSYSHQKPISNDKKESFLESLLQYAPIDDQFVKNEVKKLRKFYESKKKPPKEETVSKNCEALKNKYIKYVGKSKDDVKRELDLLENYCAYPEDIYEKFANGLSTPAYIKQKVESYWKQDGIKTKVIYSMDENLRRIKDAPLFIPPNVFYNKKDELIGLVYDYTDYISFLEDLENKRNVNIYLSIPYKKGEDKLKFKLVLGNPHKSRDSRLSIKRIFEEEYRIKGSSIGFTKNKETGKNTNLTLYLTVEVPQNKDNTLDENVVVGVDVGIAIPCVCALNNDKYTRENIGSYDTLFAKRTQFKMQRSRLNSQLKLSKGGHGRKRKLKKLELLSGKEKNYVDTECRKYASDVIKFCLKHHAKYINLEHLKGYRENPKVLAGWSFYKIQTYIEQAAEKHGIIVRKINPCYTSQICSVCGNWHPENRPKGKLGQAYFNCHNIDCKTHNTDLYKYGINADFNAARNIAMSTLFITDSDEITKKHWKEAREYYGIDESDDKEEKLNKVA

SEQ ID NO: 240 - protein, 453 aa, unidentified
MTKVVKLALINNVTDKNGNKVEYYDLNKCLWDLQKETRDLKNTVIRECWEWYGFSNDYYKLNEEYPAERDHLKREKANGTIKDYSLDGFIYSKCSKKYKLHSGNLCTTLRAASGAFKTSLKDLLRGDKSVLSYKADQPLDVQKKCIVLEYDKDTNTYYITLTLLNKAGVKCYNISDFRFKITVKDNSTRTILERCYDEVYSISSSKLIWNKKKGQWFLNLCYSFDKTETKELDKNKILGVNLGVYYPIYASISGEKDRLAISGDELIEFRNRIEARRNALKKQAAVCGDGRIGHGYKTRMKPVLNISDKIANFRDTFNHKASRKLIDFAVKNDCGIIQLENLKGVTKDTEGFLKNWSFYDLQSKIENKAKERGIKVVYIEPAYTSLRCSKCGYIHKDNHPTREQFICQECGYRTLHDYNASQNIAVKDIDKIIKAELEKMGIKKNKDEEKPEK

SEQ ID NO: 241 - protein, 453 aa, unidentified
MTKVVKLALINNVTDKNGNKVEYYDLNKCLWDLQKETRDLKNTVIRECWEWYGFSNDYYKLNEEYPAERDHLKREKANGTIKDYSLDGFIYSKYSKKYKLHSGNLCTTLRTASGAFRTSLKDILRGDKSVLSYKADQPLDVQKKCIVLEYDKDTNTYYITLTLLNKTGVKFYDIGDFRFKVTVKDNSTRTILERCYDEIYSISASKLIWNKKKGQWFLNLCYSFDKTETKELDKNKILGVNLGVYYPIYASISGEKDRLAISGDELIEFRNRIEARRNALKKQAAVCGDGRIGHGYKTRMKPVLNISDKIANFRDTFNHKASRKLIDFAVKNDCGIIQLENLKGVTKDTEGFLKNWSFYDLQSKIENKAKERGIKVVYIEPAYTSLRCSKCGYIHKDNHPTREQFICQECGYRTLHDYNASQNIAVKDIDKIIKAELEKMGIKKNKDEEKPEK

SEQ ID NO: 242 - protein, 425 aa, unidentified
MTTKTYAIKLIKPVDDSWDFAGETLRNLEYIVKRFKNKAATDQYLSIVSKDKKSAAEINTGVKQGLAELAYLNYAQECFNGAIKEAVDKVSKDFKGIVTGKSSLITYKDGQPIPVRSRQITLENDNGTYYASIGLLSREYATELGREGRQKARIKFVLSSKGNEKVVLDRILSGEYKLCDSSIQRKGNAWYLQLAHSFEAKAKTDLIANRVLGIDIGISKAVYMAVSDSPVNAFIDGGEIEQFRNKTEHRRNQMRNQLKWCSDNRKSHGRNTLLKPLEVLESKVSDFRKLTNHRYAKYVVDFAVKNQCSIIQMEDLSGINTRSAFLKRWSYFDLQTKIEDKAAAHGIKVVKVNPKYTSQRCYNCGVIAKDNRESQSVYKCECTRRTKKGVVAYKVNADLNAARNLSVLGIDKEIKAQCKAQKIAY

SEQ ID NO: 243 - protein, 469 aa, unidentified
MITVRKLKVRCEDKSFYDFLRLEQREQNKALNLAIGYIHTSNILKSNDSGAETKIIKSISKLEDKIKKLNDELNKEKITDTKREKTLKAIETTTKILEGEKKILEEGKEFRIGLDKKFNEIYIDKNNMYHVLKTQTNVQYMRTLDLVKQKVSADYSNNFIDIVTGKISLMNYKQDFPLMIDNKNINLFKENDKYYIGIMLGYELEIVLGQRVNENILELKSILDKIIEDEEMAKQNKDHVKQYRFNQSSIQFDKNNNVIFNLTFTIPQDKTFKPVEGRVLGVDLGVKYPAYMCLSDDTYKREHIGSINDFLKVRTQMQNRRRELSKALSLTNSGKGRNKKTQALKRLSEKERNFAKTYNHAISKRIVDFAKKHKCEYIHLEKLTKDGFNDRILRNWSYYELQRMVEYKADRIGIKVKYINPSYTSQKCSKCGHIDKENRQTQEKFVCTQCDFELNADHNAAINIARATE

SEQ ID NO: 244 - protein, 467 aa, unidentified
MITVRKLKLTIVGDEETRNQQYKLIRDEQYQQYRALNLCMSLLSTYNILNNWNSGAENKLNSQIEKLNKKVEKNKNDLKKDNLKENRIKKINESIKTLAKEKEKLQQEYLSSSEYRSDIDKKVKEMYIDDLYTVVQSQVNFKSKDMMSLVTQRSKKDFTTALKNGMAKGERSLTNYKRDFPLMTRGERWLKFEYDEESDDIYINWLHGIRFKVVLGYKKNENSIELRHTLHKVINKEYKICDSSMQFDRNNNLILNLTLDIPLNAKNEHIEGRTLGVDLGIKYPAYVCLSDDTYKRKSIGCAEDFIKFREQIRSRRYRLQKQLSMVKGGKGRNKKLQALDRIKDKERNFVKTYNHMISKNIVEFAKNHKCESINLEKLTKDGFPNMILSKWSYYELQNMIEYKAEREGISVKYVDPAYTSQTCSKCGYVDKENRTSQEKFKCIECGFELNADHNAAINIARSNNYVK

SEQ ID NO: 245 - protein, 557 aa, unidentified
MANKNVNDSKIKTHILTKKVQLIVDTDRFDTDEEKKAEVDRVYKYLRDSMKCQSREMNQYYMHLWMMSVARNLNDDRYSMKKYMDNIIDAVHPYLDKNNKEFNNKQKKITRDFEKKIKNLCEEYQAETEILNNDTLRELNKCFNRKKDGAYDTNFVNEMPEGLGIVMGRTVEQDFQNDCKAGLLSGIRNPRSYKINYPLIIPKSFVAYGVGSGKAMQGRGIIFPDMEYSDFHNMLFSTKNPNITYNFVHDIDFKLVFGSMKRSHELRVIFDRIKMGEYSICGSTIEINNKKKIMLNLSYEAPIYEKPSLDENTVVGVDLGMAIPAVCSLNNDDRTYKYIGDSHELEFIKKGIQAQRRSYQRNAVYNKGGHGRNRKLENLDRLKKRERNTTRTHNQRYAKQIVDFALANNAKYINLENLKGFSNNDKNKLVLRNWCYYELQQYIEIDASKYGIKVRYIYPMNTSRTCSVCGTLTTDEDVKNGVGRVSQDEFICKDPNCPSHTLYTKGPKGHKVPYFNADRNASRNIAMSEDFVKKNAKDNAFKKIDDLYEINNDIDAA

SEQ ID NO: 246 - protein, 402 aa, unidentified
MITVRKLKLTIVGDEEIRKEQYKFIRDSQYAQYQGLNLAMGVLTSSYLLSGGDVKSDYFKDAQKSLKNSNKIFNEINFGKGIDSKSYITKQVKKDFSTSLKNGLAKGERGFTNYKRDFPLMTRGRDLKFYEEDKEFYIKWVNKIVFKILTGRKDKNKVELIHTLNKVLNKEYKVSQSSLQFNKNNNLILNLTIDVKSDVKVEVIKDRVCGVGVGINTPIYVALNDILYISQSIGSNDELIKQKKQFEARKKRIRERVKNKKELNSLKEKERNWINTYNHMLSKRVVEFAKKNKCEYIYLEKINDNEFKNKVLKKWPYCELQKMIRYKAAGFGIEVKYIHSYHIFQKCSRCGYEYNKSIAIQKRFKCLNCGLEVNSDYNIARNISKYDILKDKSRITGNLVSE

SEQ ID NO: 247 - protein, 405 aa, unidentified
MLNNKFIKDEKKLQESLNKLYMEKKESETKIKRSKIDEEIKKKVNVLKKMRDKESKEATKILQQAIKINLSNTTREIINQQFNLISDTKDRITQKVSQDFKADIKNGLLRGERVLRTYKKNSPLLIRGRTLQFYRKGNDILIKWYGGITFKCIIGKRKNNNHELYILLNKILENVCKVCDSSITIGRKLILNLSVALTGFEADAPTVKGRVLGVNFGIKVPIYMSLNDKSYVQKSVGNLNDLLKLRVQLYKRKKKLENLITNAIGDKVEKLKALNRLKEKEKNMLTTYNHYLSYNIVRFAKENQVGQINIEYLPFVKAKNKALKSWPYYQLQQFIEYKAKQKKIEVKYINSYLINEKCSNCGKNITHQLNSTNVFNCKKCEYKAHLDFNISQNIALSTEYISIRK

SEQ ID NO: 248 - protein, 457 aa, unidentified
MIITKKIKIIIIGENKDKYNKFIREEYYNQNKALNVAMNHLYFLHVAEEKIRMLNNKFIKDEKKLQESLNKLYMEKKESETKIKRSKIDEEIKKKVNVLKKMRDKESKEATKILQQAIKINLSNTTREIINQQFNLISDTKDRITQKVSQDFKADIKNGLLRGERVLRTYKKNSPLLIRGRTLQFYRKGNDILIKWYGGITFKCIIGKRKNNNHELYILLNKILENVCKVCDSSITIGRKLILNLSVALTGFEADAPTVKGRVLGVNFGIKVPIYMSLNDKSYVQKSVGNLNDLLKLRVQLYKRKKKLENLITNAIGDKVEKLKALNRLKEKEKNMLTTYNHYLSYNIVRFAKENQVGQINIEYLPFVKAKNKALKSWPYYQLQQFIEYKAKQKKIEVKYINSYLINEKCSNCGKNITHQLNSTNVFNCKKCEYKAHLDFNISQNIALSTEYISIRK

SEQ ID NO: 249 - protein, 458 aa, unidentified
MIITKKIKIIIIGENKDKHNKFIREEHYNQNKALNAAMNHLYFLHVAEEKIRMLNNKFIQDEKKLQESLNKLYTEKKESKTKIKRSEIDEKIKKKVNSLKKMRDKESKEAERILQQAIKINLSNTTREIINQQFNLISDTKDRITQKVSQDFKTDIKNGLLRGDRVLRTYKKTNPLLIRGRTLQFYRKGNDILIKWYGGVTFKCIIGQRKNNNHELYILLNKILENDSKVCDSSITIGRKLILNLSVALTGFEEDIPTVKGRVLGVNFGMKVPIYMSLNDKPHVQKSVGNLNDLLKLRVQLYKRKKKMKNLIIKSIGDKAEKLKVLNRFKEKEKNILTTYNHYLSYNIVQFAKENQVGQINIEYLPLVKTKNKALKSWPYYQLQQFIEYKAKRKKIEVKYINAYLLNKKCSNCGKDTTHQSNSNNIFNCKKCQYRAPLDSNISRNIALCTEYISIRKE

SEQ ID NO: 250 - protein, 398 aa, unidentified
MKLKRTIKLVVKPSEEEKQILFKTLEEYKFAYNFVAEIGWKSKVSNSIKLHNLTYTTVREKTSLPSQLVISARMVASESLKSAFNRRKKGLKVSCPYSNNPAIRYDKRSYSVWFDREEISIATVEGRLKLKIKIPEYFKQYLNWKIRSASLKYDKRLKKFFFNIVVEKEIEEIPENDTVIGVDLGLSKLAVISTADGKINKFFDGRHIRAVSERYFAIRKKLQSKGTPSAKRHLKKLSQKEKRFRTAINHKIAKEIVSLVPAGGTIVLEELKGIRERIKVSKKERRWIHSWNFAQLQQFIEYKAQSKGIKVVYINPKYTSQRCNKCGHISKSNRKDQSHFKCSSCGYTINADLNASRNIAINYLVSQQERLGHRVASLPVWAVVNQPNVRGLANFSHS

SEQ ID NO: 251 - DNA, 222 bp, synthetic construct
  source 1..222: mol_type = other DNA
aattgcgagtataaagcaaacacagttatagtgtggtattcgcaattaatttcgggcgactcggcgtccgtgaatcgagaaagtatatgtgagtctgaatcataatcagcaatagatacactcgataaggtgaaaacaatacacatttaatccgtgtattcaactaatccttgtgtatatttgacgaaagttgcaacctatacactcgtgagagttgcgaga

SEQ ID NO: 252 - DNA, 185 bp, synthetic construct
  source 1..185: mol_type = other DNA
attcgcaattaatttcgggcgactcggcgtccgtgaatcgagaaagtatatgtgagtctgaatcataatcagcaatagatacactcgataaggtgaaaacaatacacatttaatccgtgtattcaactaatccttgtgtatatttgacgaaagttgcaacctatacactcgtgagagttgcgaga

SEQ ID NO: 253 - DNA, 134 bp, synthetic construct
  source 1..134: mol_type = other DNA
attcgcaattaatttcgggcgactcggcgtccgtgaatcgagaaagtatatgtgagtctgaatcataatcagcaatagatacactcgataaggtgaaaacaatacacatttaatccgtgtattcgaaagcgaga

SEQ ID NO: 254 - DNA, 189 bp, synthetic construct
  source 1..189: mol_type = other DNA
aattgcgagtataaagcaaacacagttatagtgtggtattcgcaattaatttcgggcgactcggcgtccgtgaatcgagaaagtatatgtgagtctgaatcataatcagcaatagatacactcgataaggtgaaaacaatacacatttaatcgaaagttgcaacctatacacttgtgagagttgcgaga

SEQ ID NO: 255 - DNA, 152 bp, synthetic construct
  source 1..152: mol_type = other DNA
attcgcaattaatttcgggcgactcggcgtccgtgaatcgagaaagtatatgtgagtctgaatcataatcagcaatagatacactcgataaggtgaaaacaatacacatttaatcgaaagttgcaacctatacacttgtgagagttgcgaga

SEQ ID NO: 256 - DNA, 93 bp, synthetic construct
  source 1..93: mol_type = other DNA
attcgcaattaatttcgggcgactcggcgtccgtgaatcgagaaagtatatgtgagtctgaatcataatcagcaatagatacagaaagcgaga

SEQ ID NO: 257 - DNA, 174 bp, synthetic construct
  source 1..174: mol_type = other DNA
cataatgattcgcactctttcgggcggctcggcgtccgtaaaccgagaaagtataagtcagtctgaatttcattcagctttagatacactcggtaaggttcaaacaatacacattcaatccgtgtattcagtccgaaagcagctgcaatctgcatatagcatgtggactgcgag

SEQ ID NO: 258 - DNA, 167 bp, synthetic construct
  source 1..167: mol_type = other DNA
attcgcactctttcgggcggctcggcgtccgtaaaccgagaaagtataagtcagtctgaatttcattcagctttagatacactcggtaaggttcaaacaatacacattcaatccgtgtattcagtccgaaagcagctgcaatctgcatatagcatgtggactgcgag

SEQ ID NO: 259 - DNA, 125 bp, synthetic construct
  source 1..125: mol_type = other DNA
attcgcactctttcgggcggctcggcgtccgtaaaccgagaaagtataagtcagtctgaatttcattcagctttagatacactcggtaaggttcaaagaaatgcgagatgtcttcgagaagacct

SEQ ID NO: 260 - DNA, 207 bp, synthetic construct
  source 1..207: mol_type = other DNA
atttattgggcgctttctcgcccataaaacgagaagtaccgctcacagtggcggcaacactcgtgaaggtagtcccatcgtttcgggtgggctgaaatctcagtcacaaaaaccgactgaggaacccttgcaactacatatttggtagatgtaaagaaagtttacatcttacctataagggtttgaaacatgtcttcgagaagacct

SEQ ID NO: 261 - DNA, 180 bp, synthetic construct
  source 1..180: mol_type = other DNA
aacgagaagtaccgctcacagtggcggcaacactcgtgaaggtagtcccatcgtttcgggtgggctgaaatctcagtcacaaaaaccgactgaggaacccttgcaactacatatttggtagatgtaaagaaagtttacatcttacctataagggtttgaaacatgtcttcgagaagacct

SEQ ID NO: 262 - DNA, 131 bp, synthetic construct
  source 1..131: mol_type = other DNA
aacgagaagtaccgctcacagtggcggcaacactcgtgaaggtagtcccatcgtttcgggtgggctgaaatctcagtcacaaaaaccgactgaggaacccttgaaaaagggttatgtcttcgagaagacct

SEQ ID NO: 263 - DNA, 199 bp, synthetic construct
  source 1..199: mol_type = other DNA
acgccactgatgtggcaggttcgcacctaatttcggggcgacttcccgccctgaaatcgagaaagtggccgtaagacgcagttctttgcgccggcaatacactcgaaaaggttaagatgcacatagtaatccgtgcatgggtcatgaaagttgcaacacgcgcgtaaggatgacttgaaggatgtcttcgagaagacct

SEQ ID NO: 264 - DNA, 181 bp, synthetic construct
  source 1..181: mol_type = other DNA
gttcgcacctaatcttggggcgacttcccgccctgaaatcgagaaagtggccgtaagacgcagttctttgcgccggcaatacactcgaaaaggttaagatgcacatagtaatccgtgcatgggtcatgaaagttgcaacacgcgcgtaaggatgacttgaaggatgtcttcgagaagacct

SEQ ID NO: 265 - DNA, 147 bp, synthetic construct
  source 1..147: mol_type = other DNA
gttcgcacctaatcttggggcgacttcccgccctgaaatcgagaaagtggccgtaagacgcagttctttgcgccggcaatacactcgaaaaggttaagatgcacatagtaatccgaaaggatgacttgaatgtcttcgagaagacct

SEQ ID NO: 266 - DNA, 199 bp, synthetic construct
  source 1..199: mol_type = other DNA
tttataacacagcagtaacaccataaactaaattaactgtttgttactgtctgcgggcgatttcacgtccgaaatatgagggtgtaaagaaatttaagtatttgcaatatccactcataaaaccgtgcatctacataagttgcgagaaagtcgcgatttgcgtaggtgcatgggatgaaaaatgtcttcgagaagacct

SEQ ID NO: 267 - DNA, 168 bp, synthetic construct
  source 1..168: mol_type = other DNA
attaactgtttgttactgtctgcgggcgatttcacgtccgaaatatgagggtgtaaagaaatttaagtatttgcaatatccactcataaaaccgtgcatctacataagttgcgagaaagtcgcgatttgcgtaggtgcatgggatgaaaaatgtcttcgagaagacct

SEQ ID NO: 268 - DNA, 128 bp, synthetic construct
  source 1..128: mol_type = other DNA
attaactgtttgttactgtctgcgggcgatttcacgtccgaaatatgagggtgtaaagaaatttaagtatttgcaatatccactcataaaaccgtgcagaaatgcatgggatgtcttcgagaagacct

SEQ ID NO: 269 - DNA, 165 bp, synthetic construct
  source 1..165: mol_type = other DNA
acatagttgttcggctttgttcgcgtaagttgtcggggcgacttcccgtccctaaatcgagaaagtggccgtaagtcttcgaatttcgaagccgacaatacactcgagaaggaaagttgcaacccgcgcgtatatgcggcttgaaggatgtcttcgagaagacct

SEQ ID NO: 270 - DNA, 147 bp, synthetic construct
  source 1..147: mol_type = other DNA
gttcgcgtaagttgtcggggcgacttcccgtccctaaatcgagaaagtggccgtaagtcttcgaatttcgaagccgacaatacactcgagaaggaaagttgcaacccgcgcgtatatgcggcttgaaggatgtcttcgagaagacct

SEQ ID NO: 271 - DNA, 109 bp, synthetic construct
  source 1..109: mol_type = other DNA
gttcgcgtaagttgtcggggcgacttcccgtccctaaatcgagaaagtggccgtaagtcttcgaatttcgaagccggaaacggcttgaaggatgtcttcgagaagacct

SEQ ID NO: 272 - DNA, 169 bp, synthetic construct
  source 1..169: mol_type = other DNA
gtgatataaaaataccgtaaggttcgcactttgttcgggcgactcgttcgtccgtaaatcgagaaagtatgcgtaagacctgatttatcgggcggcagatacactcgataaggtgaaatgttgcaactcgcacgagggtatgtactgcgagatgtcttcgagaagacct

SEQ ID NO: 273 - DNA, 148 bp, synthetic construct
  source 1..148: mol_type = other DNA
gttcgcactttgttcgggcgactcgttcgtccgtaaatcgagaaagtatgcgtaagacctgatttatcgggcggcagatacactcgataaggtgaaatgttgcaactcgcacgagggtatgtactgcgagatgtcttcgagaagacct

SEQ ID NO: 274 - DNA, 124 bp, synthetic construct
  source 1..124: mol_type = other DNA
gttcgtccgtaaatcgagaaagtatgcgtaagacctgatttatcgggcggcagatacactcgataaggtgaaatgttgcaactcgcacgagggtatgtactgcgagatgtcttcgagaagacct

SEQ ID NO: 275 - DNA, 186 bp, synthetic construct
  source 1..186: mol_type = other DNA
acataattgtaaataattattcacactttctcttgggcggctcggcgtccataaatcgagaaagtatgggtaagtctgaatttattcagcaccagatacactcggtaaggtataaactatacacattaaatcgaaagattcaatcagcatggacaggtgtcctgcgagatgtcttcgagaagacct

SEQ ID NO: 276 - DNA, 168 bp, synthetic construct
  source 1..168: mol_type = other DNA
attcacactttctcttgggcggctcggcgtccataaatcgagaaagtatgggtaagtctgaatttattcagcaccagatacactcggtaaggtataaactatacacattaaatcgaaagattcaatcagcatggacaggtgtcctgcgagatgtcttcgagaagacct

SEQ ID NO: 277 - DNA, 146 bp, synthetic construct
  source 1..146: mol_type = other DNA
attcacactttctcttgggcggctcggcgtccataaatcgagaaagtatgggtaagtctgaatttattcagcaccagatacactcggtaaggtataaactatacacattaaatcgaaagattcaatcaatgtcttcgagaagacct

SEQ ID NO: 278 - DNA, 184 bp, synthetic construct
  source 1..184: mol_type = other DNA
cttcgcaccgtctctggggcgacttcccgtcccaaaatcgagacagtggccgtcagccttcccatcgggaagcgggcaatacactcgaaaaggttaagatgcacatagtaatccgtgcatgagccacaccgaaagatgcatctcacgcgtgtccgtggcttgaaggatgtcttcgagaagacct

SEQ ID NO: 279 - DNA, 184 bp, synthetic construct
  source 1..184: mol_type = other DNA
cttcgcaccgtctctggggcgacttcccgtcccaaaatcgagacagtggccgtcagccttcccatcgggaagcgggcaatacactcgaaaaggttaagatgcacatagtaatccgtgcatgagccacaccgaaagatgcatctcaaccttgtccgtgacgggaaggatgtcttcgagaagacct

SEQ ID NO: 280 - DNA, 141 bp, synthetic construct
  source 1..141: mol_type = other DNA
cttcgcaccgtctctggggcgacttcccgtcccaaaatcgagacagtggccgtcagccttcccatcgggaagcgggcaatacactcgaaaaggttaagatgcagaaatgcatctcacgcgtgtatgtcttcgagaagacct

SEQ ID NO: 281 - DNA, 171 bp, synthetic construct
  source 1..171: mol_type = other DNA
gaaggggcgacttcccgtcccaaaatcgagatagtggtcctgattctttgatttcaaagcggacaatacactcgataaggttaagatgcacataggaatccgtgcatgggtcacaatgaaagttgcaacccgctcgctggtgtgacttgaaggatgtcttcgagaagacct

SEQ ID NO: 282 - DNA, 171 bp, synthetic construct
  source 1..171: mol_type = other DNA
gaaggggcgacttcccgtcccaaaatcgagatagtggtcctgattctttgatttcaaagcggacaatacactcgataaggttaagatgcacataggaatccgtgcatgggtcacaatgaaagttgtaacccgctcgatggtgtgacgggaaggatgtcttcgagaagacct

SEQ ID NO: 283 - DNA, 115 bp, synthetic construct
  source 1..115: mol_type = other DNA
gaaggggcgacttcccgtcccaaaatcgagatagtggtcctgattctttgatttcaaagcggacaatacactcgataaggttaaggaaacttaaaggatgtcttcgagaagacct

SEQ ID NO: 284 - DNA, 159 bp, synthetic construct
  source 1..159: mol_type = other DNA
gtttgcaacacggcaagggtgactctacaccccaaaatcgagatcagtacggcaaaaagagcgcttctgttctgccggatacactcgataaggtataaattgtatgaaagatgcaatccgcgtgcgggtgcggctgagaggatgtcttcgagaagacct

SEQ ID NO: 285 - DNA, 123 bp, synthetic construct
  source 1..123: mol_type = other DNA
agggtgactctacaccccaaaatcgagatcagtacggcaaaaagagcgcttctgttctgccggatacactcgataaggtataaattgtatgaaagatgcaatccgatgtcttcgagaagacct

SEQ ID NO: 286 - DNA, 110 bp, synthetic construct
  source 1..110: mol_type = other DNA
gtttgcaacacggcaagggtgactctacaccccaaaatcgagatcagtacggcaaaaagagcgcttctgttctgccggaaacggctgagaggatgtcttcgagaagacct

SEQ ID NO: 287 - DNA, 190 bp, synthetic construct
  source 1..190: mol_type = other DNA
ttgggggcgacttcccgtcccgaaatcgagaaagtggctgtaagtcccgttctttacgggcaggcaagacactcgaaaaggttaagatatgcacatagtaatccgtgcatgagccactgtattgtgcattgttgcagaaagttgcaactcatgcgtatgcgtggcttgaaggatgtcttcgagaagacct

SEQ ID NO: 288 - DNA, 151 bp, synthetic construct
  source 1..151: mol_type = other DNA
ttgggggcgacttcccgtcccgaaatcgagaaagtggctgtaagtcccgttctttacgggcaggcaagacactcgaaaaggttaagatatgcacatagtaatccgtgcatgagccacgaaagtggcttgaaggatgtcttcgagaagacct

SEQ ID NO: 289 - DNA, 127 bp, synthetic construct
  source 1..127: mol_type = other DNA
atcgagaaagtggctgtaagtcccgttctttacgggcaggcaagacactcgaaaaggttaagatatgcacatagtaatccgtgcatgagccacgaaagtggcttgaaggatgtcttcgagaagacct

SEQ ID NO: 290 - DNA, 185 bp, synthetic construct
  source 1..185: mol_type = other DNA
gttcggggcgacttcccgtcccaaaatcgagaaagtggctgttagccccggattatccgggcgggcaatacactcgagaaggttaagatgcacatagtaatccgtgcatgagtcactgtgctgtgcatatttgccgttgcaacttacacgtgtacgtgacttgaaggatgtcttcgagaagacct

SEQ ID NO: 291 - DNA, 150 bp, synthetic construct
  source 1..150: mol_type = other DNA
gttcggggcgacttcccgtcccaaaatcgagaaagtggctgttagccccggattatccgggcgggcaatacactcgagaaggttaagatgcacatagtaatccgtgcatgagtcacgaaagtgacttgaaggatgtcttcgagaagacct

SEQ ID NO: 292 - DNA, 125 bp, synthetic construct
  source 1..125: mol_type = other DNA
atcgagaaagtggctgttagccccggattatccgggcgggcaatacactcgagaaggttaagatgcacatagtaatccgtgcatgagtcacgaaagtgacttgaaggatgtcttcgagaagacct

SEQ ID NO: 293 - DNA, 163 bp, synthetic construct
  source 1..163: mol_type = other DNA
catctcctgtcgggggcgtcttcccgtccctaaatcgagatagcagccatttgtcttcattatttgaagacggtcttgcactcgaaaaggtcaagatgcacacaataatgaaagttgcaactcgcacgttggcactggttgaaggatgtcttcgagaagacct

SEQ ID NO: 294 - DNA, 163 bp, synthetic construct
  source 1..163: mol_type = other DNA
catctcctgtcgggggcgtcttcccgtccctaaatcgagatagcagccatttgtcttcattatttgaagacggtcttgcactcgaaaaggtcaagatgcacacaataatgaaagttgttacttgcatgttggcacttgttgaaggatgtcttcgagaagacct

SEQ ID NO: 295 - DNA, 122 bp, synthetic construct
  source 1..122: mol_type = other DNA
catctcctgtcgggggcgtcttcccgtccctaaatcgagatagcagccatttgtcttcattatttgaagacggtcttgcactcgaaaaggaaacttatcgaaggatgtcttcgagaagacct

SEQ ID NO: 296 - DNA, 157 bp, synthetic construct
  source 1..157: mol_type = other DNA
attcaggggcgacttcccgccctgaaatcgagaaagtggtcgtaagccggaagcatttccgcagacaatacactcgaaaaggttaagatatgcacatagtaatgaaagttgcaacacgcgcgaaggtgcggcttgaaggatgtcttcgagaagacct

SEQ ID NO: 297 - DNA, 117 bp, synthetic construct
  source 1..117: mol_type = other DNA
attcaggggcgacttcccgccctgaaatcgagaaagtggtcgtaagccggaagcatttccgcagacaatacactcgaaaaggttaaggaaacttgaaggatgtcttcgagaagacct

SEQ ID NO: 298 - DNA, 99 bp, synthetic construct
  source 1..99: mol_type = other DNA
attcaggggcgacttcccgccctgaaatcgagaaaattgtcgtcagacaatacactcgaaaaggtcaaggaaacttgaaggatgtcttcgagaagacct

SEQ ID NO: 299 - DNA, 161 bp, synthetic construct
  source 1..161: mol_type = other DNA
attcgagatagtggcagtaagcgcctccgcagggggctgtgcaatacactcgaaaaggttaagatgtacacatagtaatccgtgtacgaccagcattctgtgcttcttgccgctgcaagcagcatatgtacgctggttgaaggatgtcttcgagaagacct

SEQ ID NO: 300 - DNA, 128 bp, synthetic construct
  source 1..128: mol_type = other DNA
attcgagatagtggcagtaagcgcctccgcagggggctgtgcaatacactcgaaaaggttaagatgtacacatagtaatccgtgtacgaccagcgaaagctggttgaaggatgtcttcgagaagacct

SEQ ID NO: 301 - DNA, 112 bp, synthetic construct
  source 1..112: mol_type = other DNA
attcgagatagtggctgcaagtgtgcaatacactcgaaaaggttaagatgtacacatagtaatccgtgtacgaccagcgaaagctggttgaaggatgtcttcgagaagacct

SEQ ID NO: 302 - DNA, 192 bp, synthetic construct
  source 1..192: mol_type = other DNA
ggtcggatgtttccggcaatacactcggtaaggtagcgcgaatgtgaagtgtacatgaaaatataaagagattcgcgcttattgaaattactagcacaaataccgctagtacagtttacacatcaagcgaattgcaacgaaagttgcaattcgtgcgcatgtgtgaattgcaagatgtcttcgagaagacct

SEQ ID NO: 303 - DNA, 161 bp, synthetic construct
  source 1..161: mol_type = other DNA
ggtcggatgtttccggcaatacactcggtaaggtagcgcgaatgtgaagtgtacatgaaaatataaagagattcgcgcttattgaaattactagcacaaataccgctagtacagtttacacatgaaaatgtgtgaattgcaagatgtcttcgagaagacct

SEQ ID NO: 304 - DNA, 136 bp, synthetic construct
  source 1..136: mol_type = other DNA
ctcggtagtagcgcgaatgtgaagtgtacatgaaaatataaagagattcgcgcttattgaaattactagcacaaataccgctagtacagtttacacatgaaaatgtgtgaattgcaagatgtcttcgagaagacct

SEQ ID NO: 305 - DNA, 166 bp, synthetic construct
  source 1..166: mol_type = other DNA
cttcgcacatatttagggcgacttcacgtcctcaaatcgagaaagtgagcgtaagacttggcttctgtcaagcggttaatacactcgagaaggttaatatgcacatagtaatgaaagttgcaatttgtatacgagtgtgacttgaaggatgtcttcgagaagacct

SEQ ID NO: 306 - DNA, 134 bp, synthetic construct
  source 1..134: mol_type = other DNA
tagggcgacttcacgtcctcaaatcgagaaagtgagcgtacttggcttctgtcaagcggttaatacactcgagaaggttaatatgcacatagtaatgaaaattgctatttgcatacatgtcttcgagaagacct

SEQ ID NO: 307 - DNA, 116 bp, synthetic construct
  source 1..116: mol_type = other DNA
tagggcgacttcacgtcctcaaatcgagaaagtgagcgtacttggcttctgtcaagcggttaatacactcgagaaggttaatatgcagaaatgcatacatgtcttcgagaagacct

SEQ ID NO: 308 - DNA, 163 bp, synthetic construct
  source 1..163: mol_type = other DNA
taagtggatatccaacgaaatttgatataggatgtatatacgaatttcaattaccaccccaatggggtgagggcgtgttggagcgccttagtttgaggtttgatactaaaaattgagatgatggaggtcatttcgataatcaagcactcaaaaaatctactta

SEQ ID NO: 309 - DNA, 126 bp, synthetic construct
  source 1..126: mol_type = other DNA
cttcgggaatgggcgtgttggaacgccttagtttgaggtcaggattaaaaaattgacaagacgcaggtctattcagtaccgtggcactcaaaaaattcacttgattatatcaagtgaatatccaac

SEQ ID NO: 310 - DNA, 151 bp, synthetic construct
  source 1..151: mol_type = other DNA
ttgaaataaaatgaatttcaaaccccttcgggggtgggcgtgttggagcgccttaatttgaggtgcagaatccaaaaactgcgacgatgtaggtcgtttcagtctctgcgcactcaaaaaattcacttgattattcaagtgaatatccaac

SEQ ID NO: 311 - DNA, 148 bp, synthetic construct
  source 1..148: mol_type = other DNA
ctttgatataaaatagatatgaatttcattgcccattatgggctgggcgtgttggaacgccttagtttgaggtctgaaaatgaaaattgtggttgcataggcactctcgatattcaagaaagggtgttaatgccttgagtattaagtg

SEQ ID NO: 312 - DNA, 108 bp, synthetic construct
  source 1..108: mol_type = other DNA
ggtgttaatgccttgatatttaagtgaatatccaataatagatataatggatttcaagtcccttcggggacgggcgtgttggaacgccttagtttgaggtttggattc

SEQ ID NO: 313 - DNA, 137 bp, synthetic construct
  source 1..137: mol_type = other DNA
attaaaccccattatggggtgggcgtgttggaacgccttagtttgaggtttgaaaaacaaatttgggttatatttggtaatcttaatgttcaagcactcaaaaaattcacttaaattaatttaagtggatatccaac

SEQ ID NO: 314 - DNA, 140 bp, synthetic construct
  source 1..140: mol_type = other DNA
cttcggggacgggcgtgttggaacggcctcaattatgaggcttagccttagtttgaggtttggattcaaaaaatcgttggtgtgtaggcactttcgatttccaagcactcaaaaaattcacttataagtgaatatccaac

SEQ ID NO: 315 - DNA, 143 bp, synthetic construct
  source 1..143: mol_type = other DNA
gttctttgatataagtattagatatgaatttcaattcccctctgggggaagggcgtgttggaacgccttagtttgaggtttgaaaatgaaaaattgggtggtgtggaggcactcccaataaaggatgttatcggatatccaac

SEQ ID NO: 316 - DNA, 144 bp, synthetic construct
  source 1..144: mol_type = other DNA
gttctttgaggtaaaatagatatgaatttcattacccattatggggtgggcgtgttggaacgccttagtttgaggtttgaaaacagaaattaggattgcggaggcattcttgatgttcaagcaaaagagtgttaatgctttgac

SEQ ID NO: 317 - DNA, 108 bp, synthetic construct
  source 1..108: mol_type = other DNA
atttcattgcctattatgggctgggcgtgttggaacgccttagtttgaggtttgaaaacgaaaattgggaatgtagaggcactctcgatattcaagaaagcttgaatt

SEQ ID NO: 318 - DNA, 157 bp, synthetic construct
  source 1..157: mol_type = other DNA
atttctacacccattatgggttgggcgtgttggaacgccttagtttgaggtttgaagataaaaatcaaattggtggaggcctttgatattcaagcactcaaaaaattcacttatttgtgatatatagttggaaatcaacacatagtggatatccaac

SEQ ID NO: 319 - DNA, 100 bp, synthetic construct
  source 1..100: mol_type = other DNA
gttacgcatacgtcattgcgaggaggactttagtccgacgtggcaatctctattgaggtgactcatatttactatataataaggattattaagtggatat

SEQ ID NO: 320 - DNA, 144 bp, synthetic construct
  source 1..144: mol_type = other DNA
ttgaaataaaatgaatttcaaaccccttcgggggtgggcgtgttggagcgccttagtttgaggtgcagaatccaaaaactgcgacgatgtaggtcgtttcagtctctgcgcactcaaaaaattcacttgatttcaagtgaatat

SEQ ID NO: 321 - DNA, 119 bp, synthetic construct
  source 1..119: mol_type = other DNA
caacctgcgtggcctgaaggtgagaagtacatgcattaatggcgctcgcgccttgcatgtggcgctcatcgatcgctcgaaagcccgcgcggtactcgaatttgacgtgtgagcgcagg

SEQ ID NO: 322 - DNA, 145 bp, synthetic construct
  source 1..145: mol_type = other DNA
gttgagaaggaatgtgcatattagcggcgtttcgccctttgcacccgctcaccgaacaccatcaggcgggtttatatggttaattcgacctaagattcgtttaccaagaaagcttggataagcccgtttgatgggtgtattcaga

SEQ ID NO: 323 - DNA, 108 bp, synthetic construct
  source 1..108: mol_type = other DNA
gttctttgaaatatatagatatggatttcaatttcccgtttatgggatgggcgtgttggaacgccttaaaagggtgtatttgcctatgtatttaagtggatatccaac

SEQ ID NO: 324 - DNA, 115 bp, synthetic construct
  source 1..115: mol_type = other DNA
cttaatcatacagaaatgtatgcggctgatttcgcagccgaaaggtgaggattattgatatatttgaaatcccataaaggtttgagaggtatatcaaataacacaggtacgaaac

SEQ ID NO: 325 - DNA, 124 bp, synthetic construct
  source 1..124: mol_type = other DNA
ctttcgggatgggcgcgttggagcgccttggtttgaggtgaggacaccataatccgcataatgaatattgtacggatgtccctgcactcgaaaagttcacttgattatcaagtgaatatccaac

SEQ ID NO: 326 - DNA, 151 bp, synthetic construct
  source 1..151: mol_type = other DNA
caaagatatttattagggcatgttggaatgcctaagtttgaggtagaaacaaaaaaagcatttaaacagaggtttagtgttgtctttgcactcaaaaaatccgttcaaatatggctgtaaaagggtgttactgcacctagggatatccaac

SEQ ID NO: 327 - DNA, 117 bp, synthetic construct
  source 1..117: mol_type = other DNA
gtttcggtgtgtttatatggcctcaaatataaacaccgctattgtggatacaatagtacgccgaaaggtgaggattcgtcactcactaaaatccatgttaaaaagatttaacatgag

SEQ ID NO: 328 - DNA, 99 bp, synthetic construct
  source 1..99: mol_type = other DNA
attcactgtcctaagtctgaggctaaattggcactcggaaagggtaaaggcttgacactgtgttaccgtcaagacatttcacataagtgaaatgtgaat

SEQ ID NO: 329 - DNA, 109 bp, synthetic construct
  source 1..109: mol_type = other DNA
attaagtatattcgcaccatttaaggggcggctcggcgtcccaaaatcgagaaagtatatgtaaatctgaattaaaggttgcaattcgtttgtacaggtaagttgcgag

SEQ ID NO: 330 - DNA, 177 bp, synthetic construct
  source 1..177: mol_type = other DNA
acgtactatatagattataaatttgcagtgagcaagtttaacctattacataggcaaaataattggtattacttaattagaagtatattaaaaaaattatatggaatccttataggaggtacactgcaaaatttaatatttaaaaaaggtttaaacattactatgtagtatgggaat

SEQ ID NO: 331 - DNA, 171 bp, synthetic construct
  source 1..171: mol_type = other DNA
gttggttgcccttagtttgaggtagaaatccaaaaaacgtggcagttgtatctgcttcgtggctctacactcgaaaaataccatcattatttattgctataaggctcatccaaaacgaattagccgttgcaaaaggttgcaacaccttgcaaaaatggtggtatatccaac

SEQ ID NO: 332 - DNA, 119 bp, synthetic construct
  source 1..119: mol_type = other DNA
atttagcgtcctgaggccgagggctttgacctactcggcaagggttaaccctggatgttgtgtgaccgtccaggcgtttcacatagtcggtttcaaaaccaactaagtgaaatgtaaat

SEQ ID NO: 333 - DNA, 114 bp, synthetic construct
  source 1..114: mol_type = other DNA
atttcattgccatgaaattgggcgatttcacgtccataagccgagaaagtggccgcgtttgatgcggttatacactcggcaaccctgcactatgacgagtgcgagggatgaaag

SEQ ID NO: 334 - DNA, 119 bp, synthetic construct
  source 1..119: mol_type = other DNA
acatttcacgcaaaatataattgcagtaaagccaatttatatggaataagcattaaagtattgaatttacagtaatttgtagttattaaaggttatatattaactagtggaatgtaaat

SEQ ID NO: 335 - DNA, 145 bp, synthetic construct
  source 1..145: mol_type = other DNA
gttatatttattgttgactattggttagacgatgcgaaagcaaaggtttaacacaaaactcctcctatatctatatgatatagaaatgcggccgcttgcttcggccctaaattgagctgggaggtctcaagacaaagaatgcaac

SEQ ID NO: 336 - DNA, 149 bp, synthetic construct
  source 1..149: mol_type = other DNA
attcagcgactaaaggttgaggatataggattaatagctaggataacctaaagctatctataatcactcgataagggttaactctagatgttgtgttaccgtctagacatacatatagtttataaagataaactatgtgatatgtgaat

SEQ ID NO: 337 - DNA, 127 bp, synthetic construct
  source 1..127: mol_type = other DNA
attatctcaaggtccggcttattgttgtatttaagccggcgtgttctttgacatacgcgaacatttatagcaatgaaatgcggggtacgcgaaaggtcgcaccccgcgcgtatgcgggggttgaagg

SEQ ID NO: 338 - DNA, 179 bp, synthetic construct
  source 1..179: mol_type = other DNA
ggatttaccaacaatacactattactactcactaagggtaagcccaggtgttaagttaccgcctggcatactaagaatagtagtttcaaaattgcagtaaatgatatttgaaataaaacattgtaaacatagtatatataggatgttagagtatattctataataaaggttataaatct

SEQ ID NO: 339 - DNA, 157 bp, synthetic construct
  source 1..157: mol_type = other DNA
gttctataggcgatttagcgtctaaaggttgagggataagacaaaatggttaaggtttcgaccaactaactacttatccactcgataaacggtaaaaactcatactaatattctttatagataacgtggtgtgacattccaaaagtggaatgtaaat

SEQ ID NO: 340 - DNA, 139 bp, synthetic construct
  source 1..139: mol_type = other DNA
attaccactcactaagggttagcctaggtgttatgttaccgcctagcattctaagatagttaaaaaataaatttgcagtagatgatctcttataatttaagagttaaaacatagttaaagaactatgtggaatgtaaat

SEQ ID NO: 341 - DNA, 114 bp, synthetic construct
  source 1..114: mol_type = other DNA
attacgattaaattcgtagggtagatattactgcctaaggctgagggtaagggaaaatggtgttggtgtaaaacaaaggttgtacaacacccgcaattgtagtgggtataaaac

SEQ ID NO: 342 - DNA, 131 bp, synthetic construct
  source 1..131: mol_type = other DNA
gttccaaaattgaataattatattagtttattgtctatcatagatagtgtaaaaaacacagggggtagacaaaataagtaatataagtatatggtagctattaaagaatagcaccataatggaatgttaat

SEQ ID NO: 343 - DNA, 128 bp, synthetic construct
  source 1..128: mol_type = other DNA
aataagtgaaaacttacgggcgatattgcgtccgaaaagtgagggtattaccactcactaaagtctatgtaaatttatctatgtaatttgaggatttggagatatgtgattatacatagatacaaaac

SEQ ID NO: 344 - DNA, 177 bp, synthetic construct
  source 1..177: mol_type = other DNA
ttgaaataaaatgaatttcaaaccccttcgggggtgggcgtgttggagcgccttaatttgaggtgcagaatccaaaaactgcgacgatgtaggtcgtttcagtctctgcgcactcaaaaaattcacttgattatttcgatagttgtaactaccttgaatttcaagtgaatatccaac

SEQ ID NO: 345 - DNA, 173 bp, synthetic construct
  source 1..173: mol_type = other DNA
ttgaaataaaatgaatttcaaaccccttcgggggtgggcgtgttggagcgccttaatttgaggtgcagaatccaaaaactgcgacgatgtaggtcgtttcagtctctgcgcactcaaaaaattcacttgattatttcgatagaaataccttgaatttcaagtgaatatccaac

SEQ ID NO: 346 - DNA, 131 bp, synthetic construct
  source 1..131: mol_type = other DNA
aaccccttcgggggtgggcgtgttggagcgccttaatttgaggtgcagaatccaaaaactgcgacgatgtaggtcgtttcagtctctgcgcactcaaaaaattcacttgattattcaagtgaatatccaac

SEQ ID NO: 347 - DNA, 120 bp, synthetic construct
  source 1..120: mol_type = other DNA
ttgaaataaaatgaatttcaaaccccttcgggggtgggcgtgttggagcgccttaatttgaggtgcagaatcctctgcgcactcaaaaaattcacttgattattcaagtgaatatccaac

SEQ ID NO: 348 - DNA, 100 bp, synthetic construct
  source 1..100: mol_type = other DNA
aaccccttcgggggtgggcgtgttggagcgccttaatttgaggtgcagaatcctctgcgcactcaaaaaattcacttgattattcaagtgaatatccaac

SEQ ID NO: 349 - DNA, 142 bp, synthetic construct
  source 1..142: mol_type = other DNA
ttgaaataaaatgaatttcaaaccccttcgggggtgggcgtgttggagcgccttaatttgaggtgcagaatcctctgcgcactcaaaaaattcacttgattatttcgatagaaataccttgaatttcaagtgaatatccaac

SEQ ID NO: 350 - DNA, 160 bp, synthetic construct
  source 1..160: mol_type = other DNA
attaaaccccattatggggtgggcgtgttggaacgccttagtttgaggtttgaaaaacaaatttgggttatatttggtaatcttaatgttcaagcactcaaaaaattcacttaaaatttacaatggtgtaagcatcatgaaatttaagtggatatccaac

SEQ ID NO: 351 - DNA, 155 bp, synthetic construct
  source 1..155: mol_type = other DNA
attaaaccccattatggggtgggcgtgttggaacgccttagtttgaggtttgaaaaacaaatttgggttatatttggtaatcttaatgttcaagcactcaaaaaattcacttaaaatttacaatgaaaatcatgaaatttaagtggatatccaac

SEQ ID NO: 352 - DNA, 118 bp, synthetic construct
  source 1..118: mol_type = other DNA
tgggcgtgttggaacgccttagtttgaggtttgaaaaacaaatttgggttatatttggtaatcttaatgttcaagcactcaaaaaattcacttaaattaatttaagtggatatccaac

SEQ ID NO: 353 - DNA, 114 bp, synthetic construct
  source 1..114: mol_type = other DNA
attaaaccccattatggggtgggcgtgttggaacgccttagtttgaggtttgaaaaacaaatatgttcaagcactcaaaaaattcacttaaattaatttaagtggatatccaac

SEQ ID NO: 354 - DNA, 100 bp, synthetic construct
  source 1..100: mol_type = other DNA
tggggtgggcgtgttggaacgccttagtttgaggtttgaaaaacaaatatgttcaagcactcaaaaaattcacttaaattaatttaagtggatatccaac

SEQ ID NO: 355 - DNA, 132 bp, synthetic construct
  source 1..132: mol_type = other DNA
attaaaccccattatggggtgggcgtgttggaacgccttagtttgaggtttgaaaaacaaatatgttcaagcactcaaaaaattcacttaaaatttacaatgaaaatcatgaaatttaagtggatatccaac

SEQ ID NO: 356 - DNA, 144 bp, synthetic construct
  source 1..144: mol_type = other DNA
ctttcgggatgggcgcgttggagcgccttggtttgaggtgaggacaccataatccgcataatgaatattgtacggatgtccctgcactcgaaaagttcacttgatattgttgtaactgcgttgattatcaagtgaatatccaac

SEQ ID NO: 357 - DNA, 127 bp, synthetic construct
  source 1..127: mol_type = other DNA
ctttcgggatgggcgcgttggagcgccttggtttgaggtgaggacaccataatccgcataatgaatattgtacggatgtccctgcactcgaaaagttcacttgatttatttcaagtgaatatccaac

SEQ ID NO: 358 - DNA, 115 bp, synthetic construct
  source 1..115: mol_type = other DNA
tgggcgcgttggagcgccttggtttgaggtgaggacaccataatccgcataatgaatattgtacggatgtccctgcactcgaaaagttcacttgattatcaagtgaatatccaac

SEQ ID NO: 359 - DNA, 97 bp, synthetic construct
  source 1..97: mol_type = other DNA
ctttcgggatgggcgcgttggagcgccttggtttgaggtgaggacaccatgtccctgcactcgaaaagttcacttgattatcaagtgaatatccaac

SEQ ID NO: 360 - DNA, 117 bp, synthetic construct
  source 1..117: mol_type = other DNA
ctttcgggatgggcgcgttggagcgccttggtttgaggtgaggacaccatgtccctgcactcgaaaagttcacttgatattgttgtaactgcgttgattatcaagtgaatatccaac

SEQ ID NO: 361 - DNA, 127 bp, synthetic construct
  source 1..127: mol_type = other DNA
ccttcgggggtgggcgtgttggagcgccttaatttgaggtgcagaatccaaaaactgcgacgatgtaggtcgtttcagtctctgcgcactcaaaaaattcacttgattattcaagtgaatatccaac

SEQ ID NO: 362 - DNA, 129 bp, synthetic construct
  source 1..129: mol_type = other DNA
ccccttcgggggtgggcgtgttggagcgccttaatttgaggtgcagaatccaaaaactgcgacgatgtaggtcgtttcagtctctgcgcactcaaaaaattcacttgattattcaagtgaatatccaac

SEQ ID NO: 363 - DNA, 133 bp, synthetic construct
  source 1..133: mol_type = other DNA
caaaccccttcgggggtgggcgtgttggagcgccttaatttgaggtgcagaatccaaaaactgcgacgatgtaggtcgtttcagtctctgcgcactcaaaaaattcacttgattattcaagtgaatatccaac

SEQ ID NO: 364 - DNA, 135 bp, synthetic construct
  source 1..135: mol_type = other DNA
ttcaaaccccttcgggggtgggcgtgttggagcgccttaatttgaggtgcagaatccaaaaactgcgacgatgtaggtcgtttcagtctctgcgcactcaaaaaattcacttgattattcaagtgaatatccaac

SEQ ID NO: 365 - DNA, 137 bp, synthetic construct
  source 1..137: mol_type = other DNA
atttcaaaccccttcgggggtgggcgtgttggagcgccttaatttgaggtgcagaatccaaaaactgcgacgatgtaggtcgtttcagtctctgcgcactcaaaaaattcacttgattattcaagtgaatatccaac

SEQ ID NO: 366 - DNA, 139 bp, synthetic construct
  source 1..139: mol_type = other DNA
gaatttcaaaccccttcgggggtgggcgtgttggagcgccttaatttgaggtgcagaatccaaaaactgcgacgatgtaggtcgtttcagtctctgcgcactcaaaaaattcacttgattattcaagtgaatatccaac

SEQ ID NO: 367 - DNA, 114 bp, synthetic construct
  source 1..114: mol_type = other DNA
cgtgttggaacgccttagtttgaggtttgaaaaacaaatttgggttatatttggtaatcttaatgttcaagcactcaaaaaattcacttaaattaatttaagtggatatccaac

SEQ ID NO: 368 - DNA, 116 bp, synthetic construct
  source 1..116: mol_type = other DNA
ggcgtgttggaacgccttagtttgaggtttgaaaaacaaatttgggttatatttggtaatcttaatgttcaagcactcaaaaaattcacttaaattaatttaagtggatatccaac

SEQ ID NO: 369 - DNA, 120 bp, synthetic construct
  source 1..120: mol_type = other DNA
ggtgggcgtgttggaacgccttagtttgaggtttgaaaaacaaatttgggttatatttggtaatcttaatgttcaagcactcaaaaaattcacttaaattaatttaagtggatatccaac

SEQ ID NO: 370 - DNA, 122 bp, synthetic construct
  source 1..122: mol_type = other DNA
ggggtgggcgtgttggaacgccttagtttgaggtttgaaaaacaaatttgggttatatttggtaatcttaatgttcaagcactcaaaaaattcacttaaattaatttaagtggatatccaac

SEQ ID NO: 371 - DNA, 124 bp, synthetic construct
  source 1..124: mol_type = other DNA
atggggtgggcgtgttggaacgccttagtttgaggtttgaaaaacaaatttgggttatatttggtaatcttaatgttcaagcactcaaaaaattcacttaaattaatttaagtggatatccaac

SEQ ID NO: 372 - DNA, 126 bp, synthetic construct
  source 1..126: mol_type = other DNA
ttatggggtgggcgtgttggaacgccttagtttgaggtttgaaaaacaaatttgggttatatttggtaatcttaatgttcaagcactcaaaaaattcacttaaattaatttaagtggatatccaac

SEQ ID NO: 373 - DNA, 145 bp, synthetic construct
  source 1..145: mol_type = other DNA
atatatgtaactatataatccctttcgggatgggcgcgttggagcgccttggtttgaggtgaggacaccataatccgcataatgaatattgtacggatgtccctgcactcgaaaagttcacttgattatcaagtgaatatccaac

SEQ ID NO: 374 - DNA, 129 bp, synthetic construct
  source 1..129: mol_type = other DNA
aatccctttcgggatgggcgcgttggagcgccttggtttgaggtgaggacaccataatccgcataatgaatattgtacggatgtccctgcactcgaaaagttcacttgattatcaagtgaatatccaac

SEQ ID NO: 375 - DNA, 131 bp, synthetic construct
  source 1..131: mol_type = other DNA
ataatccctttcgggatgggcgcgttggagcgccttggtttgaggtgaggacaccataatccgcataatgaatattgtacggatgtccctgcactcgaaaagttcacttgattatcaagtgaatatccaac

SEQ ID NO: 376 - DNA, 113 bp, synthetic construct
  source 1..113: mol_type = other DNA
ggcgcgttggagcgccttggtttgaggtgaggacaccataatccgcataatgaatattgtacggatgtccctgcactcgaaaagttcacttgattatcaagtgaatatccaac

SEQ ID NO: 377 - DNA, 117 bp, synthetic construct
  source 1..117: mol_type = other DNA
gatgggcgcgttggagcgccttggtttgaggtgaggacaccataatccgcataatgaatattgtacggatgtccctgcactcgaaaagttcacttgattatcaagtgaatatccaac

SEQ ID NO: 378 - DNA, 119 bp, synthetic construct
  source 1..119: mol_type = other DNA
gggatgggcgcgttggagcgccttggtttgaggtgaggacaccataatccgcataatgaatattgtacggatgtccctgcactcgaaaagttcacttgattatcaagtgaatatccaac

SEQ ID NO: 379 - DNA, 133 bp, synthetic construct
  source 1..133: mol_type = other DNA
tttgatataggatgtatatacgaatttcaattaccaccccaatggggtgagggcgtgttggagcgccttagtttgaggtttgatactaaaaattgagatgatggaggtcatttcgataatcaagcactcaaaa

SEQ ID NO: 380 - DNA, 117 bp, synthetic construct
  source 1..117: mol_type = other DNA
tgggcgtgttggaacgccttagtttgaggtcaggattaaaaaattgacaagacgcaggtctattcagtaccgtggcactcaaaaaattcacttgattatatcaagtgaatatccaac

SEQ ID NO: 381 - DNA, 121 bp, synthetic construct
  source 1..121: mol_type = other DNA
atatgaatttcattgcccattatgggctgggcgtgttggaacgccttagtttgaggtctgaaaatgaaaattgtggttgcataggcactctcgatattcaagaaagggtgttaatgccttg

SEQ ID NO: 382 - DNA, 85 bp, synthetic construct
  source 1..85: mol_type = other DNA
gtgaatatccaataatagatataatggatttcaagtcccttcggggacgggcgtgttggaacgccttagtttgaggtttggattc

SEQ ID NO: 383 - DNA, 124 bp, synthetic construct
  source 1..124: mol_type = other DNA
gttggaacggcctcaattatgaggcttagccttagtttgaggtttggattcaaaaaatcgttggtgtgtaggcactttcgatttccaagcactcaaaaaattcacttataagtgaatatccaac

SEQ ID NO: 384 - DNA, 119 bp, synthetic construct
  source 1..119: mol_type = other DNA
tgaatttcaattcccctctgggggaagggcgtgttggaacgccttagtttgaggtttgaaaatgaaaaattgggtggtgtggaggcactcccaataaaggatgttatcggatatccaac

SEQ ID NO: 385 - DNA, 97 bp, synthetic construct
  source 1..97: mol_type = other DNA
tgaatttcattacccattatggggtgggcgtgttggaacgccttagtttgaggtttgaaaacagaaattaggattgcggaggcattcttgatgttca

SEQ ID NO: 386 - DNA, 93 bp, synthetic construct
  source 1..93: mol_type = other DNA
atgggctgggcgtgttggaacgccttagtttgaggtttgaaaacgaaaattgggaatgtagaggcactctcgatattcaagaaagcttgaatt

SEQ ID NO: 387 - DNA, 136 bp, synthetic construct
  source 1..136: mol_type = other DNA
tgggcgtgttggaacgccttagtttgaggtttgaagataaaaatcaaattggtggaggcctttgatattcaagcactcaaaaaattcacttatttgtgatatatagttggaaatcaacacatagtggatatccaac

SEQ ID NO: 388 - DNA, 108 bp, synthetic construct; identical to SEQ ID NO: 323
  source 1..108: mol_type = other DNA

SEQ ID NO: 389 - DNA, 133 bp, synthetic construct
  source 1..133: mol_type = other DNA
aaccccattatggggtgggcgtgttggaacgccttagtttgaggtttgaaaaacaaatttgggttatatttggtaatcttaatgttcaagcactcaaaaaattcacttaaattaatttaagtggatatccaac

SEQ ID NO: 390 - DNA, 138 bp, synthetic construct
  source 1..138: mol_type = other DNA
attaaaccccattatggggtgggcgtgttggaacgccttagtttgaggtgcagaatccaaaaactgcgacgatgtaggtcgtttcagtctctgcgcactcaaaaaattcacttaaattaatttaagtggatatccaac

SEQ ID NO: 391 - DNA, 136 bp, synthetic construct
  source 1..136: mol_type = other DNA
attaaaccccttatggggtgggcgtgttggaacgccttagtttgaggtttgaaaaacaaatttgggttatatttggtaatcttaatgttcaagcactcaaaaaattcacttagattattctaagtggatatccaac

SEQ ID NO: 392 - DNA, 133 bp, synthetic construct
  source 1..133: mol_type = other DNA
aaccccttatggggtgggcgtgttggaacgccttagtttgaggtgcagaatccaaaaactgcgacgatgtaggtcgtttcagtctctgcgcactcaaaaaattcacttagattattctaagtggatatccaac

SEQ ID NO: 393 - DNA, 131 bp, synthetic construct
  source 1..131: mol_type = other DNA
aaccccttcgggaatgggcgtgttggaacgccttagtttgaggtcaggattaaaaaattgacaagacgcaggtctattcagtaccgtggcactcaaaaaattcacttgattatatcaagtgaatatccaac

SEQ ID NO: 394 - DNA, 126 bp, synthetic construct
  source 1..126: mol_type = other DNA
cttcgggggtgggcgtgttggaacgccttagtttgaggtcaggattaaaaaattgacaagacgcaggtctattcagtaccgtggcactcaaaaaattcacttgattatatcaagtgaatatccaac

SEQ ID NO: 395 - DNA, 131 bp, synthetic construct
  source 1..131: mol_type = other DNA
aaccccttcgggggtgggcgtgttggaacgccttagtttgaggtcaggattaaaaaattgacaagacgcaggtctattcagtaccgtggcactcaaaaaattcacttgattatatcaagtgaatatccaac

SEQ ID NO: 396 - DNA, 110 bp, synthetic construct
  source 1..110: mol_type = other DNA
attcccctctgggggaagggcgtgttggaacgccttagtttgaggtttgaaaatgaaaaattgggtggtgtggaggcactcccaataaaggatgttatcggatatccaac

SEQ ID NO: 397 - DNA, 131 bp, synthetic construct
  source 1..131: mol_type = other DNA
aaccccttcgggggagggcgtgttggaacgccttagtttgaggtgcagaatccaaaaactgcgacgatgtaggtcgtttcagtctcggcacactcaaaaaattcacttggatattcaatcggatatccaac

SEQ ID NO: 398 - DNA, 131 bp, synthetic construct
  source 1..131: mol_type = other DNA
tgcccattatgggctgggcgtgttggaacgccttagtttgaggtgctgaaaatgaaaattgtggttgcataggcactctcgatctctgcgcattcaagaaattaacttgagtattcaagtgaatatccaac

SEQ ID NO: 399 - DNA, 119 bp, synthetic construct
  source 1..119: mol_type = other DNA
tgcccattatgggctgggcgtgttggaacgccttagtttgaggtctgaaaatgaaaattgtggttgcataggcactctcgatattcaagaaagggtgttaatgccttgagtattaagtg

SEQ ID NO: 400 - DNA, 126 bp, synthetic construct
  source 1..126: mol_type = other DNA
aaccccttcggggtacgggcgtgttggaacgccttagtttgaggtttggattcaaaaaatcgttggtgtgtaggcactttcgatttccaagcactcaaaaaattcacttataagtgaatatccaac

SEQ ID NO: 401 - DNA, 112 bp, synthetic construct
  source 1..112: mol_type = other DNA
tacccattatggggtgggcgtgttggaacgccttagtttgaggtttgaaaacagaaattaggattgcggaggcattcttgatgttcaagcaaaagagtgttaatgctttgac

SEQ ID NO: 402 - DNA, 130 bp, synthetic construct
  source 1..130: mol_type = other DNA
agcctattatgggctgggcgtgttggaacgccttagtttgaggtttgaaaacgaaaattgggacgatgtagatcgtttcagtctcggcactctcgaaaaattcacttgattattcaagaaagcttgaatt

SEQ ID NO: 403 - DNA, 128 bp, synthetic construct
  source 1..128: mol_type = other DNA
aacccattatgggttgggcgtgttggaacgccttagtttgaggtttgaagataaaaatcaaattggtggaggcctttgatattcaagcactcaaaaaattcacttatttgttcaagtggatatccaac

SEQ ID NO: 404 - DNA, 125 bp, synthetic construct
  source 1..125: mol_type = other DNA
aaccccttcgggggtgggcgtgttggagcgccttaagtgcagaatccaaaaactgcgacgatgtaggtcgtttcagtctctgcgcactcaaaaaattcacttgattattcaagtgaatatccaac

SEQ ID NO: 405 - DNA, 125 bp, synthetic construct
  source 1..125: mol_type = other DNA
aaccccttcgggggtgggcgtgttggagcgccttaatttgaggtgcagaatccaaaaactgcgacgatgtaggtcgtttcagtctctgcgcaaaattcacttgattattcaagtgaatatccaac

SEQ ID NO: 406 - DNA, 119 bp, synthetic construct
  source 1..119: mol_type = other DNA
aaccccttcgggggtgggcgtgttggagcgccttaagtgcagaatccaaaaactgcgacgatgtaggtcgtttcagtctctgcgcaaaattcacttgattattcaagtgaatatccaac

SEQ ID NO: 407 - DNA, 125 bp, synthetic construct
  source 1..125: mol_type = other DNA
aaccccttcgggggtgggcgtgttggagcgccttaatttgagaatccaaaaactgcgacgatgtaggtcgtttcagtctctgcgcactcaaaaaattcacttgattattcaagtgaatatccaac

SEQ ID NO: 408 - DNA, 125 bp, synthetic construct
  source 1..125: mol_type = other DNA
aaccccttcgggggtgggcgtgttggagcgccttaatttgaggtgcagaatccaaaaactgcgacgatgtaggtcgtttcagtctactcaaaaaattcacttgattattcaagtgaatatccaac

SEQ ID NO: 409 - DNA, 119 bp, synthetic construct
  source 1..119: mol_type = other DNA
aaccccttcgggggtgggcgtgttggagcgccttaatttgagaatccaaaaactgcgacgatgtaggtcgtttcagtctactcaaaaaattcacttgattattcaagtgaatatccaac

SEQ ID NO: 410 - DNA, 121 bp, synthetic construct
  source 1..121: mol_type = other DNA
aaccccttcgggggtgggcgtgttggagcgccttaatttgaggtgcagctgcgacgatgtaggtcgtttcagtctctgcgcactcaaaaaattcacttgattattcaagtgaatatccaac

SEQ ID NO: 411 - DNA, 128 bp, synthetic construct
  source 1..128: mol_type = other DNA
aaccccttcgggggtgggcgtgttggagcgccttaatttgaggtgcagaatccaaaaactgcgacgatgtaggtcgtttcagctgcgcactcaaaaaattcacttgattattcaagtgaatatccaac

SEQ ID NO: 412 - DNA, 118 bp, synthetic construct
  source 1..118: mol_type = other DNA
aaccccttcgggggtgggcgtgttggagcgccttaatttgaggtgcagctgcgacgatgtaggtcgtttcagctgcgcactcaaaaaattcacttgattattcaagtgaatatccaac

SEQ ID NO: 413 - DNA, 126 bp, synthetic construct
  source 1..126: mol_type = other DNA
aaccccttcgggggtgggcgtgttggagcgccttaatttgaggtgcagaatccaaaaaacgatgtaggtcgtttcagtctctgcgcactcaaaaaattcacttgattattcaagtgaatatccaac

SEQ ID NO: 414 - DNA, 126 bp, synthetic construct
  source 1..126: mol_type = other DNA
aaccccttcgggggtgggcgtgttggagcgccttaatttgaggtgcagaatccaaaaactgcgacgatgtaggtcgttctctgcgcactcaaaaaattcacttgattattcaagtgaatatccaac

SEQ ID NO: 415 - DNA, 121 bp, synthetic construct
  source 1..121: mol_type = other DNA
aaccccttcgggggtgggcgtgttggagcgccttaatttgaggtgcagaatccaaaaaacgatgtaggtcgttctctgcgcactcaaaaaattcacttgattattcaagtgaatatccaac

SEQ ID NO: 416 - DNA, 127 bp, synthetic construct
  source 1..127: mol_type = other DNA
aaccccttcgggggtgggcgtgttggagcgccttaatttgaggtgcagaatccaaaaactgcgtgtaggtcgtttcagtctctgcgcactcaaaaaattcacttgattattcaagtgaatatccaac

SEQ ID NO: 417 - DNA, 127 bp, synthetic construct
  source 1..127: mol_type = other DNA
aaccccttcgggggtgggcgtgttggagcgccttaatttgaggtgcagaatccaaaaactgcgacgatgtaggttcagtctctgcgcactcaaaaaattcacttgattattcaagtgaatatccaac

SEQ ID NO: 418 - DNA, 123 bp, synthetic construct
  source 1..123: mol_type = other DNA
aaccccttcgggggtgggcgtgttggagcgccttaatttgaggtgcagaatccaaaaactgcgtgtaggttcagtctctgcgcactcaaaaaattcacttgattattcaagtgaatatccaac

SEQ ID NO: 419 - DNA, 125 bp, synthetic construct
  source 1..125: mol_type = other DNA
aaccccttcgggggtgggcgtgttggagcgccttaatttgaggtgcagaatccaaaaactgcgacgatcgtttcagtctctgcgcactcaaaaaattcacttgattattcaagtgaatatccaac

SEQ ID NO: 420 - DNA, 116 bp, synthetic construct
  source 1..116: mol_type = other DNA
gggcgtgttggagcgccttaatttgaggtgcagaatccaaaaactgcgacgatgtaggtcgtttcagtctctgcgcactcaaaaaattcacttgattattcaagtgaatatccaac

SEQ ID NO: 421 - DNA, 125 bp, synthetic construct
  source 1..125: mol_type = other DNA
aaccccttcgggggtgggcgtgttggagcgccttaatttgaggtgcagaatccaaaaactgcgacgatgtaggtcgtttcagtctctgcgcactcaaaaaattcacttgattattcaagtgaata

SEQ ID NO: 422 - DNA, 110 bp, synthetic construct
  source 1..110: mol_type = other DNA
gggcgtgttggagcgccttaatttgaggtgcagaatccaaaaactgcgacgatgtaggtcgtttcagtctctgcgcactcaaaaaattcacttgattattcaagtgaata

SEQ ID NO: 423 - DNA, 20 bp, synthetic construct
  source 1..20: mol_type = other DNA
cacacacacagtgggctacc

SEQ ID NO: 424 - DNA, 20 bp, synthetic construct
  source 1..20: mol_type = other DNA
caaaagtaagattcactttc

SEQ ID NO: 425 - DNA, 20 bp, synthetic construct
  source 1..20: mol_type = other DNA
aggagtaagtctgccagcat

SEQ ID NO: 426 - DNA, 20 bp, synthetic construct
  source 1..20: mol_type = other DNA
tcggccaggaacgggggggt

SEQ ID NO: 427 - DNA, 20 bp, synthetic construct
  source 1..20: mol_type = other DNA
cagtcgggggccccacactt

SEQ ID NO: 428 - DNA, 20 bp, synthetic construct
  source 1..20: mol_type = other DNA
ctgatggtccatgtctgtta

SEQ ID NO: 429 - DNA, 20 bp, synthetic construct
  source 1..20: mol_type = other DNA
ggcggggtccagttccggga

SEQ ID NO: 430 - DNA, 20 bp, synthetic construct
  source 1..20: mol_type = other DNA
gagtctctcagctggtacac

SEQ ID NO: 431 - DNA, 20 bp, synthetic construct
  source 1..20: mol_type = other DNA
gcacgaagctctccgatgtg

SEQ ID NO: 432 - DNA, 20 bp, synthetic construct
  source 1..20: mol_type = other DNA
agtgggggtgaattcagtgt

SEQ ID NO: 433 - DNA, 20 bp, synthetic construct
  source 1..20: mol_type = other DNA
cagtctctcagctggtacac

SEQ ID NO: 434 - DNA, 20 bp, synthetic construct
  source 1..20: mol_type = other DNA
gtgtctctcagctggtacac

SEQ ID NO: 435 - DNA, 20 bp, synthetic construct
  source 1..20: mol_type = other DNA
gactctctcagctggtacac

SEQ ID NO: 436 - DNA, 20 bp, synthetic construct
  source 1..20: mol_type = other DNA
gagactctcagctggtacac

SEQ ID NO: 437 - DNA, 20 bp, synthetic construct
  source 1..20: mol_type = other DNA
gagtgtctcagctggtacac

SEQ ID NO: 438 - DNA, 20 bp, synthetic construct
  source 1..20: mol_type = other DNA
gagtcactcagctggtacac

SEQ ID NO: 439 - DNA, 20 bp, synthetic construct
  source 1..20: mol_type = other DNA
gagtctgtcagctggtacac

SEQ ID NO: 440 - DNA, 20 bp, synthetic construct
  source 1..20: mol_type = other DNA
gagtctcacagctggtacac

SEQ ID NO: 441 - DNA, 20 bp, synthetic construct
  source 1..20: mol_type = other DNA
gagtctctgagctggtacac

SEQ ID NO: 442 - DNA, 20 bp, synthetic construct
  source 1..20: mol_type = other DNA
gagtctctctgctggtacac

SEQ ID NO: 443 - DNA, 20 bp, synthetic construct
  source 1..20: mol_type = other DNA
gagtctctcacctggtacac

SEQ ID NO: 444 - DNA, 20 bp, synthetic construct
  source 1..20: mol_type = other DNA
gagtctctcaggtggtacac

SEQ ID NO: 445 - DNA, 20 bp, synthetic construct
  source 1..20: mol_type = other DNA
gagtctctcagcaggtacac

SEQ ID NO: 446 - DNA, 20 bp, synthetic construct
  source 1..20: mol_type = other DNA
gagtctctcagctcgtacac

SEQ ID NO: 447 - DNA, 20 bp, synthetic construct
  source 1..20: mol_type = other DNA
gagtctctcagctgctacac

SEQ ID NO: 448 - DNA, 20 bp, synthetic construct
  source 1..20: mol_type = other DNA
gagtctctcagctggaacac

SEQ ID NO: 449 - DNA, 20 bp, synthetic construct
  source 1..20: mol_type = other DNA
gagtctctcagctggttcac

SEQ ID NO: 450 - DNA, 20 bp, synthetic construct
  source 1..20: mol_type = other DNA
gagtctctcagctggtagac

SEQ ID NO: 451 - DNA, 20 bp, synthetic construct
  source 1..20: mol_type = other DNA
gagtctctcagctggtactc

SEQ ID NO: 452 - DNA, 20 bp, synthetic construct
  source 1..20: mol_type = other DNA
gagtctctcagctggtacag

SEQ ID NO: 453 - DNA, 20 bp, synthetic construct
  source 1..20: mol_type = other DNA
tttcccttcagctaaaataa

SEQ ID NO: 454 - DNA, 20 bp, synthetic construct
  source 1..20: mol_type = other DNA
tccgtgttccttgactctgg

SEQ ID NO: 455 - DNA, 20 bp, synthetic construct
  source 1..20: mol_type = other DNA
ttgggtcagctgttaacatc

SEQ ID NO: 456 - DNA, 20 bp, synthetic construct
  source 1..20: mol_type = other DNA
ttccccacctggagcaggct

SEQ ID NO: 457 - DNA, 20 bp, synthetic construct
  source 1..20: mol_type = other DNA
ctcgcctgtcaagtggcgtg

SEQ ID NO: 458 - DNA, 20 bp, synthetic construct
  source 1..20: mol_type = other DNA
tgaacctgggtgaagtccca

SEQ ID NO: 459 - DNA, 20 bp, synthetic construct
  source 1..20: mol_type = other DNA
ttaatgttaataacttgctt

SEQ ID NO: 460 - DNA, 20 bp, synthetic construct
  source 1..20: mol_type = other DNA
acattaacaagaagcatttg

SEQ ID NO: 461 - DNA, 20 bp, synthetic construct
  source 1..20: mol_type = other DNA
gaacatccgcgaaatgatac

SEQ ID NO: 462 - DNA, 20 bp, synthetic construct
  source 1..20: mol_type = other DNA
ccagggcgaagtggggaggt

SEQ ID NO: 463 - DNA, 20 bp, synthetic construct
  source 1..20: mol_type = other DNA
ccctggctacctcccctacc

SEQ ID NO: 464 - DNA, 20 bp, synthetic construct
  source 1..20: mol_type = other DNA
cacacacacagtgggctacc

SEQ ID NO: 465 - DNA, 20 bp, synthetic construct
  source 1..20: mol_type = other DNA
gctcagcaggcacctgcctc

SEQ ID NO: 466 - DNA, 20 bp, synthetic construct
  source 1..20: mol_type = other DNA
gggattcctggtgccagaaa

SEQ ID NO: 467 - DNA, 20 bp, synthetic construct
  source 1..20: mol_type = other DNA
tgcagccgccgctccagagc

SEQ ID NO: 468 - DNA, 20 bp, synthetic construct
  source 1..20: mol_type = other DNA
acaggcgagtaacagacatg

SEQ ID NO: 469 - DNA, 20 bp, synthetic construct
  source 1..20: mol_type = other DNA
acgtactgatgttaacagct

SEQ ID NO: 470 - DNA, 20 bp, synthetic construct
  source 1..20: mol_type = other DNA
ggtcagctgttaacatcagt

SEQ ID NO: 471 - protein, 529 aa, unidentified
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKIGEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 472 - DNA, 130 bp, synthetic construct
  source 1..130: mol_type = other DNA
aaccccttcgggggtgggcgtgttggaacgccttagtttgaggtcaggattaaaaaattgacaagacgcaggtctattcagtacctggcactcaaaaaattcacttgattatatcaagtgaatatccaac

SEQ ID NO: 473 - DNA, 130 bp, synthetic construct
  source 1..130: mol_type = other DNA
aaccccttcgggggtgggcgtgttggaacgccttagtttgaggtcaggattaaaaaactgacaagacgcaggtctattcagtacctggcactcaaaaaattcacttgattatatcaagtgaatatccaac

SEQ ID NO: 474 - DNA, 128 bp, synthetic construct
  source 1..128: mol_type = other DNA
aaccccttcgggggtgggcgtgttggaacgccttagtttgaggtcaggattaaaaaactgcaagacgcaggtctatcagtacctggcactcaaaaaattcacttgattatatcaagtgaatatccaac

SEQ ID NO: 475 - DNA, 128 bp, synthetic construct
  source 1..128: mol_type = other DNA
aaccccttcgggggtgggcgtgttggaacgccttagtttgaggtcaggattaaaaaactgcgagacgcaggtctttcagtacctggcactcaaaaaattcacttgattatatcaagtgaatatccaac

SEQ ID NO: 476 - DNA, 130 bp, synthetic construct
  source 1..130: mol_type = other DNA
aaccccttcgggggtgggcgtgttggaacgccttagtttgaggtcaggattaaaaaactgcgacgacgcaggtcgtttcagtacctggcactcaaaaaattcacttgattatatcaagtgaatatccaac

SEQ ID NO: 477 - DNA, 132 bp, synthetic construct
  source 1..132: mol_type = other DNA
aaccccttcgggggtgggcgtgttggaacgccttagtttgaggtgcaggattaaaaaactgcgacgacgcaggtcgtttcagtacctgcgcactcaaaaaattcacttgattatatcaagtgaatatccaac

SEQ ID NO: 478 - DNA, 131 bp, synthetic construct
  source 1..131: mol_type = other DNA
aaccccttcgggggtgggcgtgttggaacgccttagtttgaggtgcaggattaaaaaactgcgacgacgcaggtcgtttcagtacctgcgcactcaaaaaattcacttgattattcaagtgaatatccaac

SEQ ID NO: 479 - DNA, 153 bp, synthetic construct
  source 1..153: mol_type = other DNA
aaccccttcgggggtgggcgtgttggagcgccttaatttgaggtgcagaatccaaaaactgcgacgatgtaggtcgtttcagtctctgcgcactcaaaaaattcacttgattatttcgatagaaataccttgaatttcaagtgaatatccaac

SEQ ID NO: 480 - DNA, 157 bp, synthetic construct
  source 1..157: mol_type = other DNA
aaccccttcgggggtgggcgtgttggagcgccttaatttgaggtgcagaatccaaaaactgcgacgatgtaggtcgtttcagtctctgcgcactcaaaaaattcacttgattatttcgatagttgtaactaccttgaatttcaagtgaatatccaac

SEQ ID NO: 481 - DNA, 131 bp, synthetic construct
  source 1..131: mol_type = other DNA
aaccccttcgggggtgggcgtgttggagcgccttaacgcgaggtgcagaatccaaaaactgcgacgatgtaggtcgtttcagtctctgcgcactcgcgaaattcacttgattattcaagtgaatatccaac

SEQ ID NO: 482 - DNA, 130 bp, synthetic construct
  source 1..130: mol_type = other DNA
aaccccttcgggggtgggcgtgttggagcgccttaacgcgaggtgcagaatccaaaaactgcgacgatgtaggtcgtttcagtctctgcgcctcgcgaaattcacttgattattcaagtgaatatccaac

SEQ ID NO: 483 - DNA, 20 bp, synthetic construct
  source 1..20: mol_type = other DNA
agaacaccatagctgccaat

SEQ ID NO: 484 - DNA, 20 bp, synthetic construct
  source 1..20: mol_type = other DNA
aggagtaagtctgccagcat

SEQ ID NO: 485 - DNA, 20 bp, synthetic construct
  source 1..20: mol_type = other DNA
tgaagagctgatgctcgccc

SEQ ID NO: 486 - DNA, 20 bp, synthetic construct
  source 1..20: mol_type = other DNA
tggtgctgatggaggagacc

SEQ ID NO: 487 - DNA, 20 bp, synthetic construct
  source 1..20: mol_type = other DNA
ggatttggggttttgtcctc

SEQ ID NO: 488 - DNA, 20 bp, synthetic construct
  source 1..20: mol_type = other DNA
cagccacgtctacagcaggg

SEQ ID NO: 489 - DNA, 20 bp, synthetic construct
  source 1..20: mol_type = other DNA
ctgacgacagccgtggtgct

SEQ ID NO: 490 - DNA, 55 bp, synthetic construct
  source 1..55: mol_type = other DNA
acactctttccctacacgacgctcttccgatctgtctgcaatggacagctccaag

SEQ ID NO: 491 - DNA, 54 bp, synthetic construct
  source 1..54: mol_type = other DNA
gactggagttcagacgtgtgctcttccgatctccagtgtgaaggagtgagaggg

SEQ ID NO: 492 - DNA, 60 bp, synthetic construct
  source 1..60: mol_type = other DNA
acactctttccctacacgacgctcttccgatcttaacttcctttattttccttacagggt

SEQ ID NO: 493 - DNA, 67 bp, synthetic construct
  source 1..67: mol_type = other DNA
gactggagttcagacgtgtgctcttccgatctctttaatattgattgttttacattaacctttcaac

SEQ ID NO: 494 - DNA, 57 bp, synthetic construct
  source 1..57: mol_type = other DNA
acactctttccctacacgacgctcttccgatctgagtcccaggcgtccatgtccttc

SEQ ID NO: 495 - DNA, 56 bp, synthetic construct
  source 1..56: mol_type = other DNA
gactggagttcagacgtgtgctcttccgatctcataccttggagcaacggcggaag

SEQ ID NO: 496 - DNA, 53 bp, synthetic construct
  source 1..53: mol_type = other DNA
acactctttccctacacgacgctcttccgatctgtgcatgcaacaagacaaca

SEQ ID NO: 497 - DNA, 52 bp, synthetic construct
  source 1..52: mol_type = other DNA
gactggagttcagacgtgtgctcttccgatctgaagccagggaagaggtcat

SEQ ID NO: 498 - DNA, 55 bp, synthetic construct
  source 1..55: mol_type = other DNA
acactctttccctacacgacgctcttccgatctatgctaaagaggtggggctcag

SEQ ID NO: 499 - DNA, 54 bp, synthetic construct
  source 1..54: mol_type = other DNA
gactggagttcagacgtgtgctcttccgatctccccatcggaagatcctctctg

SEQ ID NO: 500 - DNA, 52 bp, synthetic construct
  source 1..52: mol_type = other DNA
acactctttccctacacgacgctcttccgatctgttccagagtctatcaccg

SEQ ID NO: 501 - DNA, 50 bp, synthetic construct
  source 1..50: mol_type = other DNA
gactggagttcagacgtgtgctcttccgatctgcttacccagaggcaaag

SEQ ID NO: 502 - DNA, 55 bp, synthetic construct
  source 1..55: mol_type = other DNA
acactctttccctacacgacgctcttccgatctttgtctgcagctcctacctctg

SEQ ID NO: 503 - DNA, 54 bp, synthetic construct
  source 1..54: mol_type = other DNA
gactggagttcagacgtgtgctcttccgatcttcttcctgagctgctaacacgg

SEQ ID NO: 504 - protein, 7 aa, synthetic construct
PKKKRKV

SEQ ID NO: 505 - protein, 16 aa, synthetic construct
KRPAATKKAGQAKKKK

SEQ ID NO: 506 - DNA, 59 bp, synthetic construct
  source 1..59: mol_type = other DNA
acactctttccctacacgacgctcttccgatctgtaatgagcaaccttgagggatcagg

SEQ ID NO: 507 - DNA, 58 bp, synthetic construct
  source 1..58: mol_type = other DNA
gactggagttcagacgtgtgctcttccgatctctcatggcaaaagcagtaatcagaac

SEQ ID NO: 508 - RNA, 151 nt, synthetic construct
  source 1..151: mol_type = other RNA
aaccccttcgggggtgggcgtgttggagcgccttaatttgaggtgcagaatccaaaaactgcgacgatgtaggtggtttcagtctctgcgcactcaaaaaattcacttgattattcaagtgaatatccaaccacacacacagtgggctacc

SEQ ID NO: 509 - RNA, 123 nt, synthetic construct
  source 1..123: mol_type = other RNA
aaccccttcgggggtgggcgtgttggagcgccttaatttgagctcaaaaaattcacttgattatttcgatagaaataccttgaatttcaagtgaatatccaaccacacacacagtgggctacc

SEQ ID NO: 510 - RNA, 127 nt, synthetic construct
  source 1..127: mol_type = other RNA
aaccccttcgggggtgggcgtgttggagcgccttaatttgagctcaaaaaattcacttgattatttcgatagttgtaactaccttgaatttcaagtgaatatccaaccacacacacagtgggctacc